(19) United States
(12) Patent Application Publication
(10) Pub. No.: US 2024/0101540 A1
(43) Pub. Date: Mar. 28, 2024

(54) TARGETED PROTEIN DEGRADATION OF PARP14 FOR USE IN THERAPY

(71) Applicant: Ribon Therapeutics, Inc., Cambridge, MA (US)

(72) Inventors: Nicholas Robert Perl, Somerville, MA (US); Kevin Wayne Kuntz, Woburn, MA (US); Jennifer Downing, Cambridge, MA (US)

(21) Appl. No.: 18/227,639

(22) Filed: Jul. 28, 2023

Related U.S. Application Data
(60) Provisional application No. 63/443,170, filed on Feb. 3, 2023.
(60) Provisional application No. 63/420,307, filed on Oct. 28, 2022.
(60) Provisional application No. 63/393,522, filed on Jul. 29, 2022.

(51) Int. Cl. C07D 405/14; A61K 45/06; C07D 401/14; C07D 471/04; C07D 471/10; C07D 487/08

(52) U.S. Cl. CPC C07D 405/14; A61K 45/06; C07D 401/14; C07D 471/04; C07D 471/10; C07D 487/08; C07B 2200/05

(57) ABSTRACT

The present invention relates to quinazolinones and related compounds which degrade PARP14 and are useful, for example, in the treatment of cancer and inflammatory diseases.

### FIELD OF THE INVENTION

The present invention relates to quinazolinones and related compounds which cause intracellular proteolysis of PARP14 and are useful in the treatment of cancer and inflammatory diseases.

### BACKGROUND OF THE INVENTION

Poly(ADP-ribose) polymerases (PARPs) are members of a family of seventeen enzymes that regulate fundamental cellular processes including gene expression, protein degradation, and multiple cellular stress responses (Vyas S, et al. Nat Rev Cancer. 2014 Jun. 5; 14(7):502-509). The ability of cancer cells to survive under stress is a fundamental cancer mechanism and an emerging approach for novel therapeutics. One member of the PARP family, PARP1, has already been shown to be an effective cancer target in connection to cellular stress induced by DNA damage, either induced by genetic mutation or with cytotoxic chemotherapy, with three approved drugs in the clinic and several others in late stage development (Ohmoto A, et al. OncoTargets and Therapy. 2017; Volume 10:5195).

The seventeen members of the PARP family were identified in the human genome based on the homology within their catalytic domains (Vyas S, et al. Nat Commun. 2013 Aug. 7; 4:2240). However, their catalytic activities fall into 3 different categories. The majority of PARP family members catalyze the transfer of mono-ADP-ribose units onto their substrates (monoARTs), while others (PARP1, PARP2, TNKS, TNKS2) catalyze the transfer of poly-ADP-ribose units onto substrates (polyARTs). Finally, PARP13 is thus far the only PARP for which catalytic activity could not be demonstrated either in vitro or in vivo.

PARP14 is a cytosolic as well as nuclear monoART. It was originally identified as BAL2 (B Aggressive Lymphoma 2), a gene associated with inferior outcome of diffuse large B cell lymphoma (DLBCL), together with two other monoARTs (PARP9 or BAL1 and PARP15 or BAL3) (Aguiar R C, et al. Blood. 2000 Dec. 9; 96(13):4328-4334 and Juszczynski P, et al. Mol Cell Biol. 2006 Jul. 1; 26(14):5348-5359). PARP14, PARP9 and PARP15 are also referred to as macro-PARPs due to the presence of macro-domains in their N-terminus. The genes for the three macroPARPs are located in the same genomic locus suggesting co-regulation. Indeed, the gene expression of PARP14 and PARP9 is highly correlated across normal tissues and cancer types. PARP14 is overexpressed in tumors compared to normal tissues, including established cancer cell lines in comparison to their normal counterparts. Literature examples of cancers with high PARP14 expression are DLBCL (Aguiar R C T, et al. J Biol Chem. 2005 Aug. 1; 280(40):33756-33765), multiple myeloma (MM) (Barbarulo A, et al. Oncogene. 2012 Oct. 8; 32(36):4231-4242) and hepatocellular carcinoma (HCC) (Iansante V, et al. Nat Commun. 2015 Aug. 10; 6:7882). In MM and HCC cell lines RNA interference (RNAi) mediated PARP14 knockdown inhibits cell proliferation and survival. Other studies show that the enzymatic activity of PARP14 is required for survival of prostate cancer cell lines in vitro (Bachmann S B, et al. Mol Cancer. 2014 May 27; 13:125).

PARP14 is an interferon stimulated gene with its mRNA increased by stimulation of various cell systems with all types of interferon (I, II and III; www.interferome.org). PARP14 has been identified as a downstream regulator of IFN-7 and IL-4 signaling, influencing transcription downstream of STAT1 (in the case of IFN-7) (Iwata H, et al. Nat Commun. 2016 Oct. 31; 7:12849) or STAT6 (in the case of IL-4) (Goenka S, et al. Proc Natl Acad Sci USA. 2006 Mar. 6; 103(11):4210-4215; Goenka S, et al. J Biol Chem. 2007 May 3; 282(26):18732-18739; and Mehrotra P, et al. J Biol Chem. 2010 Nov. 16; 286(3):1767-1776). Parp14−/− knockout (KO) mice have reduced marginal zone B cells, and the ability of IL-4 to confer B cell survival in vitro was reduced as well in the Parp14 KO setting (Cho S H, et al. Blood. 2009 Jan. 15; 113(11):2416-2425). This decreased survival signaling was linked mechanistically to decreased abilities of Parp14 KO B cells to sustain metabolic fitness and to increased Mcl-1 expression. Parp14 KO can extend survival in the Eμ-Myc lymphoma model, suggesting a role of PARP14 in Myc-driven lymphomagenesis (Cho S H, et al. Proc Natl Acad Sci USA. 2011 Sep. 12; 108(38):15972-15977).

Gene expression data point towards roles of PARP14 in human B cell lymphoma as well. The BAL proteins, including PARP14, are highly expressed in host response (HR) DLBCLs, a genomically defined B cell lymphoma subtype characterized by inflammatory infiltrate of T and dendritic cells and presence of an IFN-7 gene signature (Molecular profiling of diffuse large B-cell lymphoma identifies robust subtypes including one characterized by host inflammatory response. Monti S, et al. Blood. 2005; 105(5):1851).

Due to its role downstream of IL-4 and IFN-7 signaling pathways PARP14 has been implicated in T helper cell and macrophage differentiation. Genetic PARP14 inactivation in macrophages skews to a pro-inflammatory M1 phenotype associated with antitumor immunity while reducing a pro-tumor M2 phenotype. M1 gene expression, downstream of IFN-7, was found to be increased while M2 gene expression, downstream of IL-4, was decreased with PARP14 knockout or knockdown in human and mouse macrophage models. Similarly, genetic PARP14 knockout has been shown to reduce a Th2 T helper cell phenotype in the setting of skin and airway inflammation, again pertaining to the regulatory role of PARP14 in IL-4 signal transduction (Mehrotra P, et al. J Allergy Clin Immunol. 2012 Jul. 25; 131(2):521 and Krishnamurthy P, et al. Immunology. 2017 Jul. 27; 152(3):451-461).

PARP14 promotes signaling by Type 2 helper T cells (TH2) and Type 17 helper T cells (TH17) cytokines by acting as a coactivator of STAT6- and STAT3-driven transcription (Goenka et al. 2006 PMID 16537510, Mehrotra et al. 2015 PMID 26222149). PARP14 is upregulated in tissues with inflammatory disease, such as the skin lesions in atopic dermatitis or psoriasis patients (He et al. 2021 PMID: 32709423) or in endobronchial biopsies from mild atopic asthma patients (Yick et al. 2013 PMID: 23314903). Either genetic deletion or catalytic inhibition of PARP14 has been shown to block IL 4/STAT6 signaling in macrophages in vitro (Iwata et al. 2016 PMID 27796300, Schenkel et al. 2021 PMID: 33705687) and to suppress pathogenic changes associated with allergic airway disease in mouse models (Cho et al. 2013 PMID: 23956424, Mehrotra et al. 2013 PMID: 22841009, Eddie et al. 2022 PMID: 35817532). Antibodies and small molecules suppressing TH2/TH17-cytokine signaling and alarmins are either approved or being investigated as treatments for multiple inflammatory diseases such as atopic dermatitis, asthma, chronic rhinosinusitis, and eosinophilic esophagitis (Sastre et al. 2018, PMID: 29939132, Lyly et al. 2020 PMID: 33322143, Ahn et al. 2021 PMID: 33911806, Ahn et al. 2021 PMID: 33935450). Given the upregulation of PARP14 in tissues with inflammatory diseases, the central role of PARP14 in TH2- and TH17-driven cytokine signaling, and the common underlying biology of many inflammatory diseases, small molecules targeting PARP14 could be promising therapeutics for a broad range of inflammatory diseases.

Most clinically used pharmaceutical agents are based upon small-molecule inhibition of protein function. However, alternative approaches that provide for protein degradation, rather than inhibition, also have the potential to provide clinical efficacy. Accordingly, targeted protein degradation through ubiquitination of protein targets has emerged as an effective strategy in drug discovery. Heterobifunctional small molecules, which simultaneously bind to target proteins and recruit an ubiquitin ligase (e.g., ubiquitin E3 ligase) have been shown to result in the target protein's ubiquitination and degradation (Bondeson, D. P., et al. Nat Chem Biol. 2015 11(8):611-617). Examples of these small molecules, which can bind to both PARP14 and ubiquitin E3 ligase, have been described in PCT Patent Publication WO 2020/257416.

There is a need for the development of new drugs, such as small molecules that can bind to both PARP14 and ubiquitin E3 ligase to cause PARP14 degradation, which are useful in the treatment of various diseases, including cancer and inflammatory diseases.

### SUMMARY OF THE INVENTION

The present invention is directed to a compound of Formula (I):

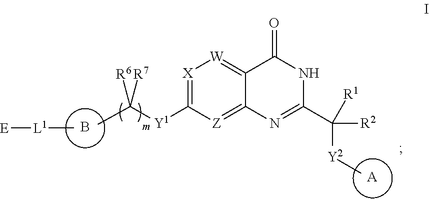

or a pharmaceutically acceptable salt thereof, wherein constituent members are defined below.

The present invention is further directed to a pharmaceutical composition comprising a compound of Formula (I), or a pharmaceutically acceptable salt thereof, and at least one pharmaceutically acceptable carrier.

The present invention is further directed to a method of degrading PARP14, comprising contacting a compound of Formula (I), or a pharmaceutically acceptable salt thereof, with PARP14.

The present invention is further directed to a method of treating a disease or disorder in a patient in need of treatment, where the disease or disorder is characterized by overexpression or increased activity of PARP14, comprising administering to the patient a therapeutically effective amount of a compound Formula (I), or a pharmaceutically acceptable salt thereof.

The present invention is further directed to a method of treating cancer in a patient in need thereof comprising administering to said patient a therapeutically effective amount of a compound of Formula (I), or a pharmaceutically acceptable salt thereof.

The present invention is further directed to a method of treating an inflammatory disease in a patient in need of treatment comprising administering to said patient a therapeutically effective amount of a compound of Formula (I), or a pharmaceutically acceptable salt thereof.

The present invention also provides uses of the compounds described herein in the manufacture of a medicament for use in therapy. The present disclosure also provides the compounds described herein for use in therapy.

### BRIEF DESCRIPTION OF THE DRAWINGS

FIG. 1 is a graph showing the dose-dependent reduction of Alternaria-induced cell infiltration in BALF following treatment with Compound 64.

FIG. 2A is a graph demonstrating that Compound 64 significantly reduces eosinophils in BALF in a dose-dependent manner starting from 100 mg/kg.

FIG. 2B is a graph demonstrating that Compound 64 significantly reduces cytokine IL-33 in BALF in a dose-dependent manner starting from 100 mg/kg.

FIG. 2C is a graph demonstrating that Compound 64 significantly reduces cytokine IL-4 in BALF in a dose-dependent manner starting from 100 mg/kg.

FIG. 2D is a graph demonstrating that Compound 64 significantly reduces cytokine IL-5 in BALF in a dose-dependent manner starting from 100 mg/kg.

### DETAILED DESCRIPTION

The present disclosure provides, inter alia, a compound of Formula (I):

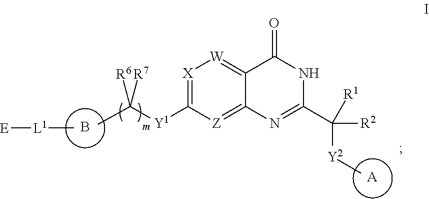

or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, —O—, and —(C₂₋₄ alkynyl)-;
Y² is selected from bond, —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and —(NR³)CH₂—;
Ring A is selected from 6-10 membered aryl, 5-10 membered heteroaryl, C₃₋₁₄ cycloalkyl, and 4-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋₈ dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋ₛ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{a1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹—C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, N^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²—C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³—C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4} NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4}; R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{e2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R⁴, NR^{c4}S(O)₂R^{b4}, NR^{c4}S(O)₂NR^{c4}R^{d4}, and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase; and
L¹ is a linker, which is covalently linked to moiety Q and to moiety E;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

The present disclosure provides, inter alia, a compound of Formula (I):

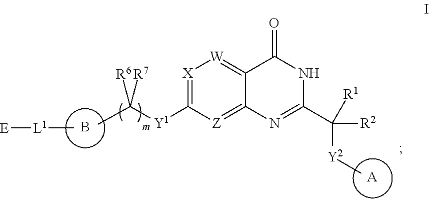

or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, and —O—;
Y² is selected from bond, —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and —(NR³)CH₂—;
Ring A is selected from 6-10 membered aryl, 5-10 membered heteroaryl, C₃₋₁₄ cycloalkyl, and 4-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋₈ dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋ₛ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1} NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl,
C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4} NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4}; R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{e2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R⁴, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R⁴, NR^{c4}S(O)₂R^{b4}, NR^{c4}S(O)₂NR^{c4}R^{d4}, and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase; and
L¹ is a linker, which is covalently linked to moiety Q and to moiety E;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

The present disclosure provides, inter alia, a compound of Formula (I):

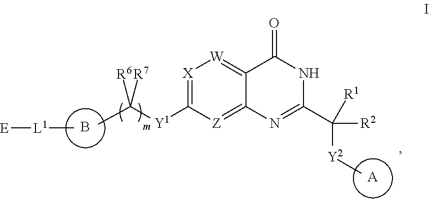

or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, and —O—;
Y² is selected from —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and —(NR³)CH₂—;
Ring A is selected from 6-10 membered aryl, 5-10 membered heteroaryl, C₃₋₁₄ cycloalkyl, and 4-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋₈ dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋ₛ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1} NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1} NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, N^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl,
C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{e2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R², NR^{c2}R^{d2}, NR^{c2}C(O)R^{b2} NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4} NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R⁴, NR^{c4}S(O)₂R^{b4}, NR^{c4}S(O)₂NR^{c4}R^{d4} and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase; and
L¹ is a linker, which is covalently linked to moiety Q and to moiety E;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

In some embodiments, W is CR^{W}. In some embodiments, W is N.

In some embodiments, X is CR^{X}. In some embodiments, X is N.

In some embodiments, Z is CR^{Z}. In some embodiments, Z is N.

In some embodiments, Y¹ is —O—. In some embodiments, Y¹ is —CR⁴R⁵—. In some embodiments, Y¹ is —NR³—. In some embodiments, Y¹ is —O— or —CR⁴R⁵—. In some embodiments, Y¹ is —O— or —NR³—.

In some embodiments, Y¹ is —(C₂₋₄ alkynyl)-. In some embodiments, Y¹ is —(C₂ alkynyl)-. In some embodiments, Y¹ is —O—, —NR³—, or —(C₂₋₄ alkynyl)-. In some embodiments, Y¹ is —O—, —NR³—, or —(C₂ alkynyl)-.

In some embodiments, Y² is S. In some embodiments, Y² is —CH₂—. In some embodiments, Y¹ is —S— or —CH₂—. In some embodiments, Y² is selected from —S—, —S(O)—, —S(O)₂—, —CH₂—, —O—, and —N(R³)—. In some embodiments, Y² is selected from —SCH₂—, —S(O)CH₂—, —S(O)₂CH₂—, —CH₂CH₂—, —OCH₂—, and —(NR³)CH₂—. In some embodiments, Y² is —S—, —CH₂—, or —O—. In some embodiments, Y² is bond. In some embodiments, Y² is bond, —S—, —CH₂—, or —O—.

In some embodiments, Ring A is 4-18 membered heterocycloalkyl, wherein Ring A is optionally substituted by 1, 2, 3, or 4 R^{A}.

In some embodiments, Ring A is 4-7 membered heterocycloalkyl, wherein Ring A is optionally substituted by 1, 2, 3, or 4 R^{A}. In some embodiments, Ring A is 4-7 membered heterocycloalkyl, wherein Ring A is optionally substituted by 1 or 2 R^{A}. In some embodiments, Ring A is 4-7 membered heterocycloalkyl.

In some embodiments, Ring A is tetrahydro-2H-pyranyl or piperidinyl, each optionally substituted by 1, 2, 3, or 4 R^{A}. In some embodiments, Ring A is tetrahydro-2H-pyranyl, optionally substituted by 1, 2, 3, or 4 R^{A}. In some embodiments, Ring A is piperidinyl, optionally substituted by 1, 2, 3, or 4 R^{A}. In some embodiments, Ring A is tetrahydro-2H-pyranyl or piperidinyl, each optionally substituted by 1 or 2 R^{A}. In some embodiments, Ring A is tetrahydro-2H-pyranyl, optionally substituted by 1 or 2 R^{A}. In some embodiments, Ring A is piperidinyl, optionally substituted by R^{A}.

In some embodiments, Ring A is tetrahydro-2H-pyran-4-yl or 1-methylpiperidin-4-yl. In some embodiments, Ring A is tetrahydro-2H-pyran-4-yl. In some embodiments, Ring A is 1-methylpiperidin-4-yl. In some embodiments, Ring A is tetrahydro-2H-pyran-4-yl, 1-methylpiperidin-4-yl, or 1-(2,2,2-trifluoroethyl)piperidin-4-yl.

In some embodiments, Ring A is C₃₋₁₄ cycloalkyl, wherein Ring A is optionally substituted by 1, 2, 3, or 4 R^{A}. In some embodiments, Ring A is cyclohexyl, wherein Ring A is optionally substituted by 1, 2, 3, or 4 R^{A}. In some embodiments, Ring A is 4-hydroxycyclohexyl. In some embodiments, Ring A is cyclopropyl, cyclobutyl, cyclopentyl, or cyclohexyl, wherein Ring A is optionally substituted by 1, 2, 3, or 4 R^{A}. In some embodiments, Ring A is cyclopropyl, cyclobutyl, cyclopentyl, or hydroxycyclohexyl.

In some embodiments, Ring A is 4-7 membered heterocycloalkyl or C₃₋₁₄ cycloalkyl, wherein Ring A is optionally substituted by 1, 2, 3, or 4 R^{A}. In some embodiments, Ring A is 4-7 membered heterocycloalkyl or C₃₋₁₄ cycloalkyl, wherein Ring A is optionally substituted by 1 or 2R^{A}. In some embodiments, Ring A is tetrahydro-2H-pyran-4-yl, 1-methylpiperidin-4-yl, 1-(2,2,2-trifluoroethyl)piperidin-4-yl, or 4-hydroxycyclohexyl. In some embodiments, Ring A is tetrahydro-2H-pyran-4-yl, 1-methylpiperidin-4-yl, or 1-(2,2,2-trifluoroethyl)piperidin-4-yl.

In some embodiments, Ring A is tetrahydro-2H-pyran-4-yl, 1-methylpiperidin-4-yl, 1-(2,2,2-trifluoroethyl)piperidin-4-yl, piperazinyl, cyclopropyl, cyclobutyl, cyclopentyl, or 4-hydroxycyclohexyl.

In some embodiments, Ring A is tetrahydro-2H-pyran-4-yl, 1-methylpiperidin-4-yl, 1-(2,2,2-trifluoroethyl)piperidin-4-yl, piperazinyl, cyclopropyl, cyclobutyl, cyclopentyl, cyclohexyl, or 4-hydroxycyclohexyl.

In some embodiments, Ring B is C₃₋₇ cycloalkyl or 4-7 membered heterocycloalkyl, wherein Ring B is optionally substituted by 1, 2, 3, or 4 R^{B}. In some embodiments, Ring B is C₃₋₇ cycloalkyl, wherein Ring B is optionally substituted by 1, 2, 3, or 4 R^{B}. In some embodiments, Ring B is 4-7 membered heterocycloalkyl, wherein Ring B is optionally substituted by 1, 2, 3, or 4 R^{B}. In some embodiments, Ring B is piperidinyl, cyclobutyl, or cyclohexyl, each of which is optionally substituted by 1, 2, 3, or 4 R^{B}. In some embodiments, Ring B is piperidinyl, cyclobutyl, or cyclohexyl.

In some embodiments, Ring B is C₃₋₇ cycloalkyl or 4-7 membered heterocycloalkyl, wherein Ring B is optionally substituted by 1 or 2 R^{B}. In some embodiments, Ring B is C₃₋₇ cycloalkyl, wherein Ring B is optionally substituted by 1 or 2 R^{B}. In some embodiments, Ring B is 4-7 membered heterocycloalkyl, wherein Ring B is optionally substituted by 1 or 2 R^{B}.

In some embodiments, Ring B is piperidinyl optionally substituted by 1, 2, 3, or 4 R^{B}. In some embodiments, Ring B is piperidinyl optionally substituted by 1 or 2 R^{B}. In some embodiments, Ring B is piperidinyl. In some embodiments, Ring B is piperazinyl. In some embodiments, Ring B is piperidinyl or piperazinyl optionally substituted by 1, 2, 3, or 4 R^{B}.

In some embodiments, Ring B is C₃₋₇ cycloalkyl, wherein Ring B is optionally substituted by 1, 2, 3, or 4 R^{B}. In some embodiments, Ring B is cyclobutyl or cyclohexyl. In some embodiments, Ring B is cyclopropyl, cyclobutyl, or cyclohexyl In some embodiments, Ring B is cyclobutyl, cyclohexyl, piperidinyl, or piperazinyl, each optionally substituted by 1, 2, 3, or 4 R^{B}. In some embodiments, Ring B is cyclobutyl, cyclohexyl, piperidinyl, or piperazinyl.

In some embodiments, Ring B is piperidinyl, piperazinyl, or azetidinyl, each of which is optionally substituted by 1, 2, 3, or 4 R^{B}.

In some embodiments, R¹ and R² are each H. In some embodiments, R¹ is H. In some embodiments, R² is H.

In some embodiments, R⁴ and R⁵ are each H. In some embodiments, R⁴ is H. In some embodiments, R⁵ is H.

In some embodiments, R⁶ and R⁷ are each H. In some embodiments, R⁶ is H. In some embodiments, R⁷ is H.

In some embodiments, each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, and C₁₋₆ haloalkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1} NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, NR^{c1}R^{d1}, NR^{c1}C(O)R^{b1}, S(O)NR^{c1}R^{d1}, S(O)₂R^{b1}, and S(O)₂NR^{c1}R^{d1}.

In some embodiments, each R^{A} is independently selected from halo, C₁₋₆ alkyl, C₁₋₆ haloalkyl, CN, NO₂, or OR^{a1}. In some embodiments, each R^{A} is C₁₋₆ alkyl. In some embodiments, each R^{A} is C₁₋₆ alkyl or C₁₋₆ haloalkyl. In some embodiments, R^{A} is methyl. In some embodiments, R^{A} is methyl, OH, or 2,2,2-trifluoroethyl.

In some embodiments, each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, and C₁₋₆ haloalkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}.

In some embodiments, each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, NR^{c2}R^{d2}, NR^{c2}C(O)R^{b2}, S(O)NR^{c2}R^{d2}, S(O)₂R^{b2}, and S(O)₂NR^{c2}R^{d2}.

In some embodiments, each R^{B} is independently selected from halo, C₁₋₆ alkyl, C₁₋₆ haloalkyl, CN, NO₂, or OR^{a2}. In some embodiments, each R^{B} is independently selected from halo, C₁₋₆ alkyl, and C₁₋₆ haloalkyl. In some embodiments, each R^{B} is independently selected from halo and C₁₋₆ haloalkyl. In some embodiments, each R^{B} is independently selected from halo. In some embodiments, each R^{B} is F.

In some embodiments, R^{W}, R^{X}, and R^{Z} are each, independently, selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, and C₁₋₆ haloalkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{W} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{W} is selected from H, halo, C₁₋₆ alkyl, C₂₋₆ alkenyl, C₂₋₆ alkynyl, C₁₋₆ haloalkyl, CN, NO₂, and OR^{a3}. In some embodiments, R^{W} is selected from H, halo, and C₁₋₆ haloalkyl. In some embodiments, R^{W} is F.

In some embodiments, R^{X} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{X} is selected from C₆₋₁₀ aryl and 5-10 membered heteroaryl, wherein said C₆₋₁₀ aryl and 5-10 membered heteroaryl are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, OR^{a3}, and SR^{a3}

In some embodiments, R^{X} is selected from H, halo, C₁₋₆ alkyl, C₂₋₆ alkenyl, C₂₋₆ alkynyl, C₁₋₆ haloalkyl, CN, OR^{a3}, and C₆₋₁₀ aryl. In some embodiments, R^{X} is H. In some embodiments, R^{X} is H or halo. In some embodiments, R^{X} is H or F. In some embodiments, R^{X} is F.

In some embodiments, R^{Z} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

In some embodiments, R^{Z} is selected from C₆₋₁₀ aryl and 5-10 membered heteroaryl, wherein said C₆₋₁₀ aryl and 5-10 membered heteroaryl are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, OR^{a3}, and SR^{a3}

In some embodiments, R^{Z} is selected from H, halo, C₁₋₆ alkyl, C₂₋₆ alkenyl, C₂₋₆ alkynyl, C₁₋₆ haloalkyl, CN, OR^{a3}, and C₆₋₁₀ aryl. In some embodiments, R^{X} is H.

In some embodiments, m is 1. In some embodiments, m is 0. In some embodiments, m is 2. In some embodiments, m is 0 or 1. In some embodiments, m is 1 or 2.

In some embodiments, L¹ is linked to ring B through a covalent bond to ring B.

In some embodiments, L¹ is a chain of 1 to 40, 1 to 30, 1 to 25, 1 to 20, 1 to 15, 1 to 10, or 1 to 5 chain atoms, which is optionally substituted with 1-3 R^{q} substituents, and wherein one or more chain carbon atoms of L¹ can be oxidized to form a carbonyl (C═O), and wherein one or more N and S chain atoms can each be optionally oxidized to form an amine oxide, sulfoxide or sulfonyl group; and
each R^{q} is independently selected from OH, CN, —COOH, NH₂, halo, C₁₋₆ haloalkyl, C₁₋₆ alkyl, C₁₋₆ alkoxy, C₁₋₆ haloalkoxy, C₁₋₆ alkylthio, phenyl, 5-6 membered heteroaryl, 4-6 membered heterocycloalkyl, C₃₋₆ cycloalkyl, NH(C₁₋₆ alkyl) and N(C₁₋₆ alkyl)₂, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5- or 6-membered heteroaryl or 4-6 membered heterocycloalkyl. In some embodiments, R^{q} is independently selected from OH, CN, —COOH, NH₂, halo, C₁₋₆ haloalkyl, C₁₋₆ alkyl, C₁₋₆ alkoxy, C₁₋₆ haloalkoxy, NH(C₁₋₆ alkyl) and N(C₁₋₆ alkyl)₂.

In some embodiments, L¹ is selected from the following:
(i) bond, such that ring B is directly attached to moiety E;
(ii) —(C₁₋₄ alkyl)-;
(iii) —(C₂₋₄ alkenyl)-;
(iv) —(C₂₋₄ alkynyl);
(V) —(C₂₋₄ alkynyl)-(G₃)-.
(vi) the following structure:

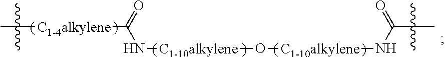

(vii) the following structure:

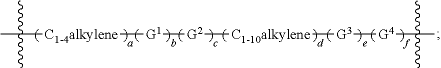

and
   (viii) the following structure:

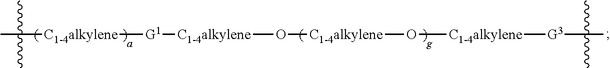

wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl;
G³ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G⁴ is 4-10 membered heterocycloalkyl, which is optionally substituted with C₁₋₆ alkyl, hydroxyl, C₁₋₆ alkoxy, or halo;
each R^{G} is independently selected from H, methyl, and ethyl;
a is 0 or 1;
b is 0 or 1;
c is 0 or 1;
d is 0 or 1;
e is 0 or 1;
f is 0 or 1;
g is 0, 1, or 2;
   wherein at least one of b, c, e, and f is 1.

In some embodiments, L¹ is selected from the following:
(i) bond, such that ring B is directly attached to moiety E;
(ii) —(C₁₋₄ alkyl)-;
(iii) —(C₂₋₄ alkenyl)-;
(iv) —(C₂₋₄ alkynyl);
(V) —(C₂₋₄ alkynyl)-(G³)-.
(vi) the following structure:

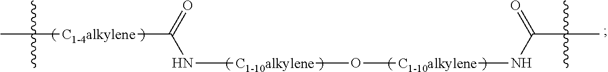

(vii) the following structure:

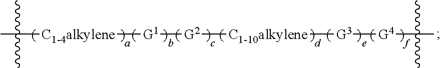

and
(viii) the following structure:

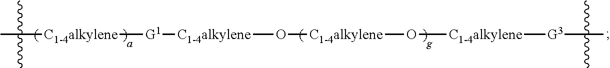

wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl, each of which is optionally substituted by halo, C₁₋₆ alkyl, or C₁₋₆ haloalkyl;
G³ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G⁴ is 4-10 membered heterocycloalkyl;
each R^{G} is independently selected from H, methyl, and ethyl;
a is 0 or 1;
b is 0 or 1;
cis 0 or 1;
d is 0 or 1;
e is 0 or 1;
f is 0 or 1;
g is 0, 1, or 2;
wherein at least one of b, c, e, and f is 1.

In some embodiments, L¹ is selected from the following:
(viii) bond, such that ring B is directly attached to moiety E;
(ix) —(C₁₋₄ alkyl)-;
(x) —(C₂₋₄ alkenyl)-;
(xi) —(C₂₋₄ alkynyl);
(xii) —(C₂₋₄ alkynyl)-(G³)-;
(xiii) the following structure:

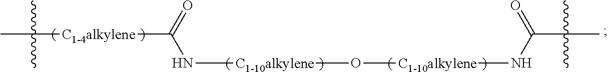

(xiv) the following structure:

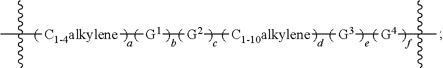

and
(viii) the following structure:

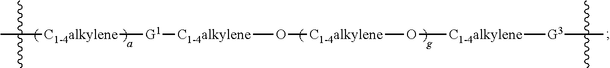

wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl;
G³ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G⁴ is 4-10 membered heterocycloalkyl;
each R^{G} is independently selected from H, methyl, and ethyl;
a is 0 or 1;
bis 0 or 1;
c is 0 or 1;
d is 0 or 1;
e is 0 or 1;
f is 0 or 1;
g is 0, 1, or 2;
wherein at least one of b, c, e, and f is 1.

In some embodiments, L¹ is selected from the following:
(i) bond, such that ring B is directly attached to moiety E;
(ii) —(C₁₋₄ alkyl)-;
(iii) —(C₂₋₄ alkenyl)-;
(iv) —(C₂₋₄ alkynyl);
(V) —(C₂₋₄ alkynyl)-(G³)-.
(vi) the following structure:

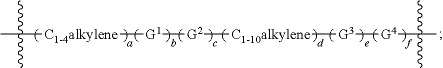

(vii) the following structure:

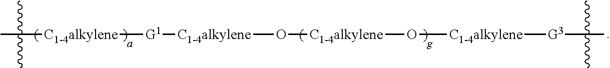

In some embodiments, L¹ is a bond, such that ring B is directly attached to moiety E.

In some embodiments, L¹ is —(C₁₋₄ alkyl)-.

In some embodiments, L¹ is —(C₂₋₄ alkenyl)-.

In some embodiments, L¹ is —(C₂₋₄ alkynyl)-.

In some embodiments, L¹ is —(C₂₋₄ alkynyl)-(G³)-.

In some embodiments, L¹ has the following structure:

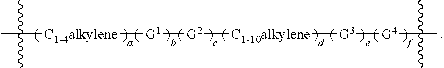

In some embodiments, L¹ is

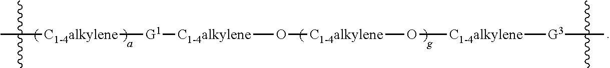

In some embodiments, G¹ is —NR^{G}C(O)— or —C(O)—. In some embodiments, G¹ is —NR^{G}C(O)—. In some embodiments, G¹ is —C(O)—. In some embodiments, G¹ is —NR^{G}C(O)—, —C(O)—, or —O—. In some embodiments, G¹ is —O—

In some embodiments, G² is 4-10 membered heterocycloalkyl, which is optionally substituted by halo or C₁₋₆ haloalkyl. In some embodiments, G² is 4-10 membered heterocycloalkyl, which is optionally substituted by C₁₋₆ haloalkyl. In some embodiments, G² is 4-10 membered heterocycloalkyl. In some embodiments, G² is piperidinyl, piperazinyl, or azetidinyl. In some embodiments, G² is piperidinyl or piperazinyl. In some embodiments, G² is piperidinyl. In some embodiments, G² is piperazinyl. In some embodiments, G² is azetidinyl. In some embodiments, G² is piperidinyl, piperazinyl, azetidinyl, 3,6-diazabicyclo[3.1.1]heptanyl, or 3,4-dihydroquinazolin-7-yl.

In some embodiments, G² is piperidinyl, piperazinyl, azetidinyl, pyridinyl, 3,6-diazabicyclo[3.1.1]heptanyl, 3,4-dihydroquinazolin-7-yl, cyclobutyl, 3,8-diazabicyclo[3.2.1]octanyl, cyclohexyl, (trifluoromethyl)piperazinyl, 7-azaspiro[3.5]nonanyl, piperazin-1-yl-2,2,3,3,5,5,6,6-d8, 2-azaspiro[3.5]nonanyl, or 7-azaspiro[3.5]nonanyl. In some embodiments, G² is piperidinyl, piperazinyl, azetidinyl, 3,6-diazabicyclo[3.1.1]heptanyl, 3,4-dihydroquinazolin-7-yl, cyclobutyl, 3,8-diazabicyclo[3.2.1]octanyl, cyclohexyl, (trifluoromethyl)piperazinyl, 7-azaspiro[3.5]nonanyl, piperazin-1-yl-2,2,3,3,5,5,6,6-d8, 2-azaspiro[3.5]nonanyl, or 7-azaspiro[3.5]nonanyl. In some embodiments, G² is piperidinyl, piperazinyl, azetidinyl, 3,6-diazabicyclo[3.1.1]heptanyl, 3,4-dihydroquinazolin-7-yl, 3,8-diazabicyclo[3.2.1]octanyl, (trifluoromethyl)piperazinyl, 7-azaspiro[3.5]nonanyl, piperazin-1-yl-2,2,3,3,5,5,6,6-d8, 2-azaspiro[3.5]nonanyl, or 7-azaspiro[3.5]nonanyl. In some embodiments, G² is cyclobutyl or cyclohexyl.

In some embodiments, G² is piperidinyl, piperazinyl, azetidinyl, pyridinyl, 3,6-diazabicyclo[3.1.1]heptanyl, 3,4-dihydroquinazolin-7-yl, cyclobutyl, 3,8-diazabicyclo[3.2.1]octanyl, cyclohexyl, (trifluoromethyl)piperazinyl, 7-azaspiro[3.5]nonanyl, piperazin-1-yl-2,2,3,3,5,5,6,6-d8, 2-azaspiro[3.5]nonanyl, 7-azaspiro[3.5]nonanyl, or pyrrolidinyl.

In some embodiments, G² is C₃₋₇ cycloalkyl. In some embodiments, G² is cyclobutyl.

In some embodiments, G² is piperidinyl, piperazinyl, azetidinyl, 3,6-diazabicyclo[3.1.1]heptanyl, 3,4-dihydroquinazolin-7-yl, or cyclobutyl.

In some embodiments, G³ is —NR^{G}— or —O—. In some embodiments, G³ is —NR^{G}—. In some embodiments, G³ is —O—.

In some embodiments, G⁴ is piperidinyl or piperazinyl. In some embodiments, G⁴ is piperidinyl. In some embodiments, G⁴ is piperazinyl. In some embodiments, G⁴ is azetidinyl. In some embodiments, G⁴ is azetidinyl, piperidinyl, or piperazinyl In some embodiments, a is 0. In some embodiments, a is 1.

In some embodiments, b is 0. In some embodiments, b is 1.

In some embodiments, c is 0. In some embodiments, c is 1.

In some embodiments, d is 0. In some embodiments, d is 1.

In some embodiments, e is 0. In some embodiments, e is 1.

In some embodiments, f is 0. In some embodiments, f is 1.

In some embodiments, g is 0. In some embodiments, g is 1.

In some embodiments, R^{G} is H. In some embodiments, R^{G} is methyl.

Ubiquitin ligase binding moieties and linkers are known and well-described in the art, for example: Bondeson, D. P., et al. Nat Chem Biol. 2015 11(8):611-617; An S, et al. EBioMedicine 2018 36:553-562; Paiva S-L. et al, Curr. Op. in Chem. Bio. 2010, 50:111-119; and International Patent Application Publication No. WO 2017/197056, each of which is incorporated by reference in its entirety.

In some embodiments, E is a Von Hippel-Lindau (VHL) E3 ubiquitin ligase binding moiety, a MDM2 E3 ubiquitin ligase binding moiety, a cereblon E3 ubiquitin ligase binding moiety, or an inhibitor of apoptosis proteins (IAP) E3 ubiquitin ligase binding moiety, each of which has an IC₅₀ of less than about 10 μM as determined in a binding assay. For example, E is a cereblon E3 ubiquitin ligase binding moiety. E can be a Von Hippel-Lindau (VHL) E3 ubiquitin ligase binding moiety. E can be a MDM2 E3 ubiquitin ligase binding moiety. E can be an IAP E3 ubiquitin ligase binding moiety.

In some embodiments, E is an E3 ubiquitin ligase binding moiety that binds to cereblon.

In some embodiments, E comprises a chemical group derived from an imide, a thioimide, an amide, or a thioamide.

In some embodiments, E is thalidomide, lenalidomide, pomalidomide, analogs thereof, isosteres thereof, or derivatives thereof.

In some embodiments, E is selected from the following:

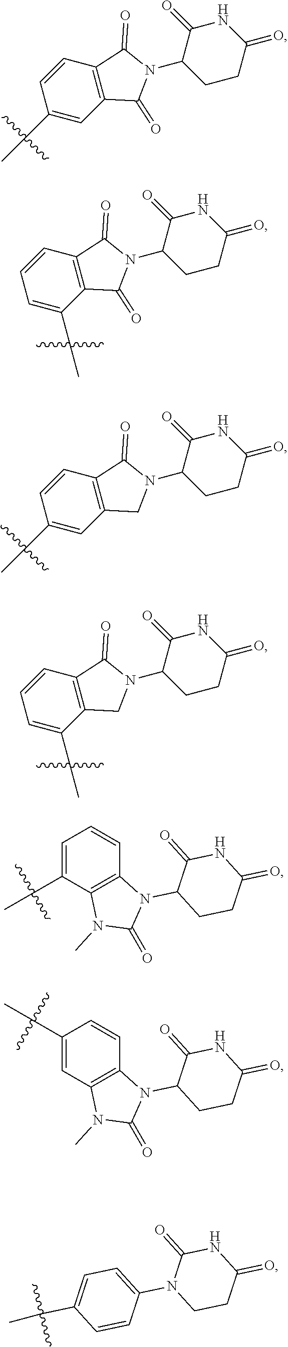

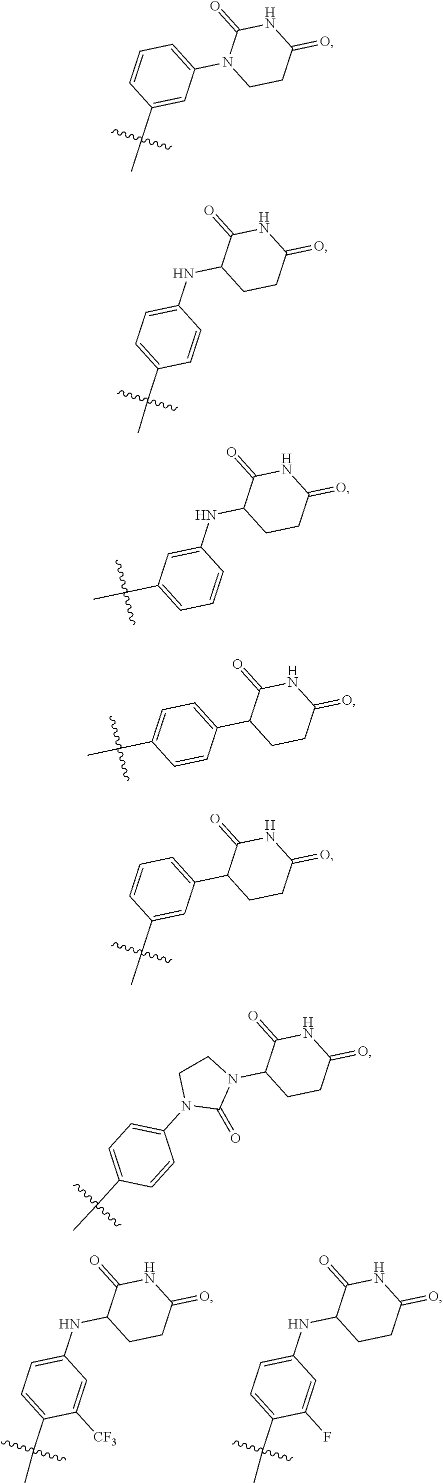

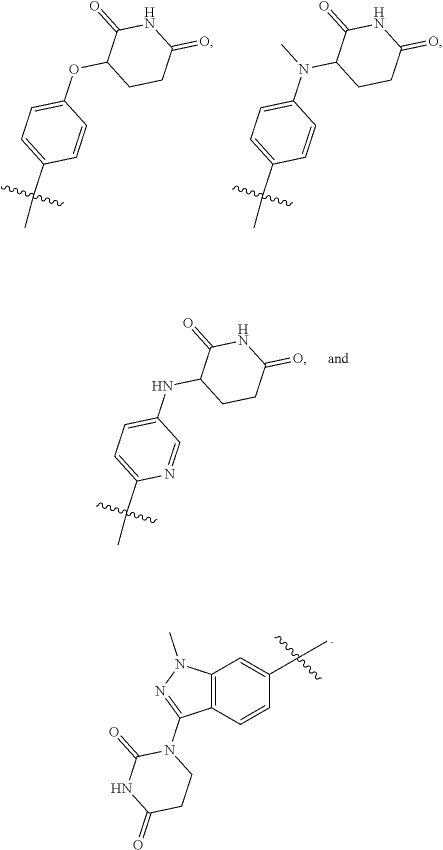 [SMILES: *c1ccc(OC2CCC(=O)NC2=O)cc1 ; *c1ccc(N(C)C2CCC(=O)NC2=O)cc1 ; *c1ccc(NC2CCC(=O)NC2=O)cn1 ; "and" ; *c1ccc2c(N3CCC(=O)NC3=O)nn(C)c2c1]

In some embodiments, E is selected from the following:

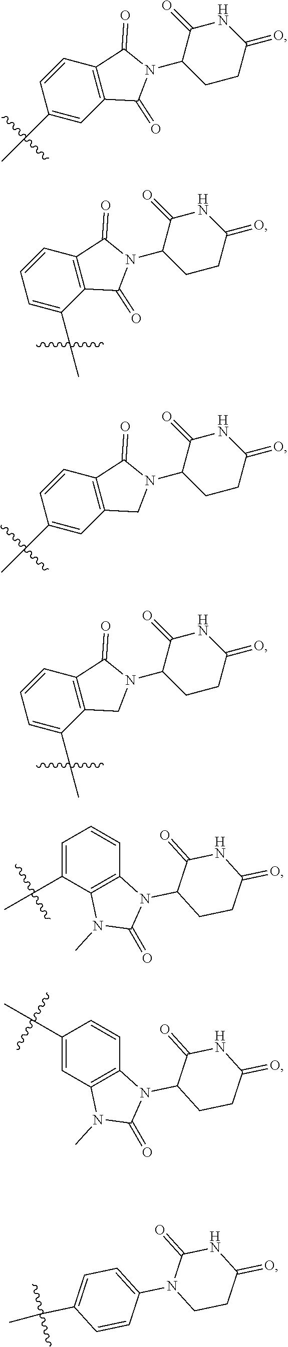

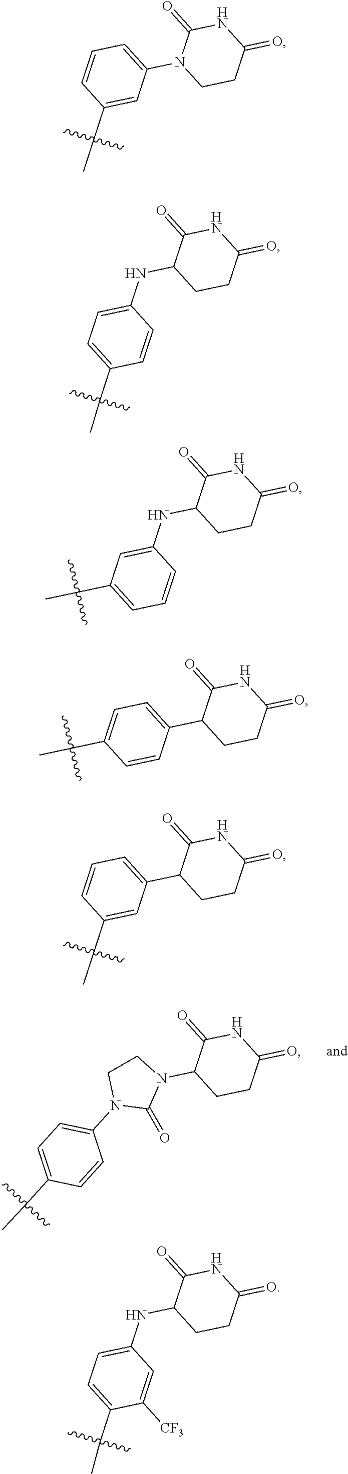

wherein the wavy lines represent the point of attachment to group L¹.

In some embodiments, E is selected from:

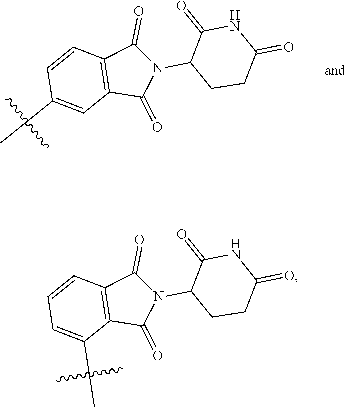 [SMILES: *c1ccc2c(c1)C(=O)N(C1CCC(=O)NC1=O)C2=O ; *c1cccc2c1C(=O)N(C1CCC(=O)NC1=O)C2=O]

wherein the wavy lines represent the point of attachment to group L¹.

In some embodiments, E is:

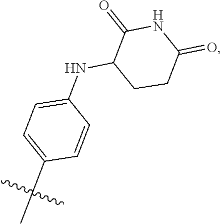 [SMILES: *c1ccc(NC2CCC(=O)NC2=O)cc1]

wherein the wavy line represents the point of attachment to group L¹.

In some embodiments, E is:

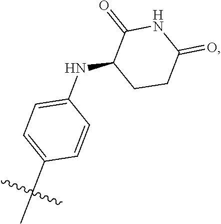 [SMILES: *c1ccc(N[C@@H]2CCC(=O)NC2=O)cc1]

wherein the wavy line represents the point of attachment to group L¹.

In some embodiments, E is:

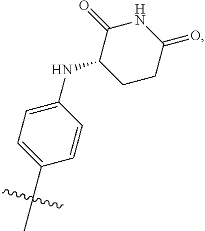 [SMILES: *c1ccc(N[C@H]2CCC(=O)NC2=O)cc1]

wherein the wavy line represents the point of attachment to group L¹.

In some embodiments, E is:

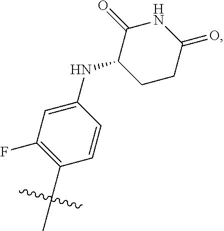 [SMILES: *c1ccc(N[C@H]2CCC(=O)NC2=O)cc1F]

wherein the wavy line represents the point of attachment to group L¹.

In some embodiments, E is:

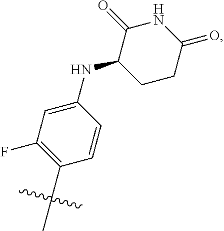 [SMILES: *c1ccc(N[C@@H]2CCC(=O)NC2=O)cc1F]

wherein the wavy line represents the point of attachment to group L¹.

In some embodiments, E is:

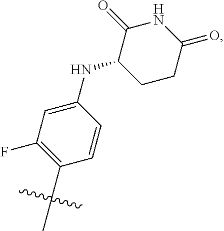 [SMILES: *c1ccc(N[C@H]2CCC(=O)NC2=O)cc1F]

wherein the wavy line represents the point of attachment to group L¹.

In some embodiments, E is selected from the following:

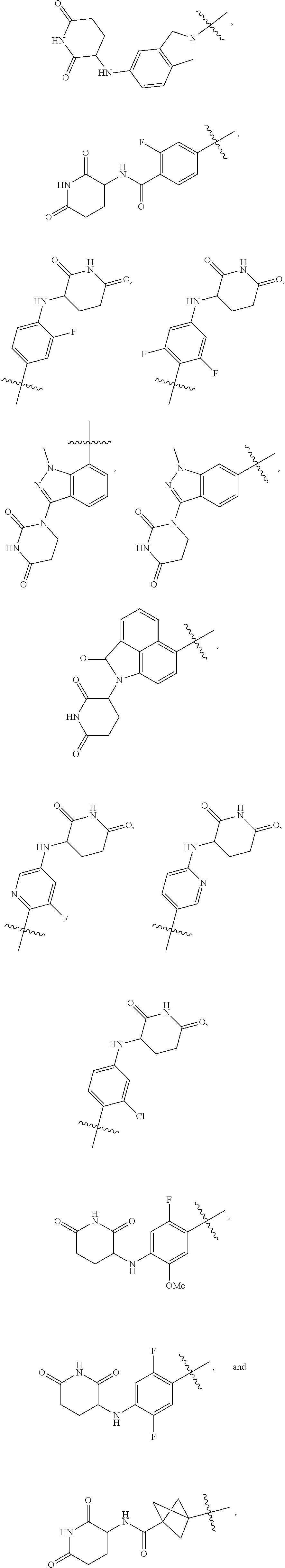

wherein the wavy line represents the point of attachment to group L¹.

In some embodiments, E is selected from the following:

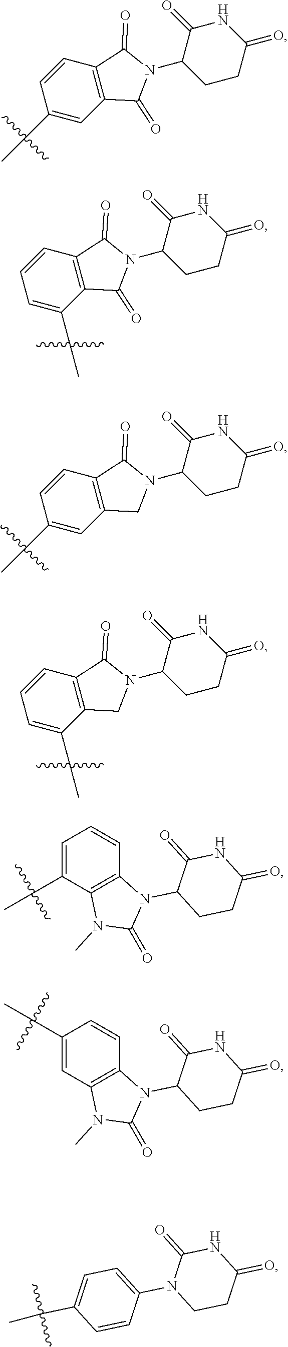

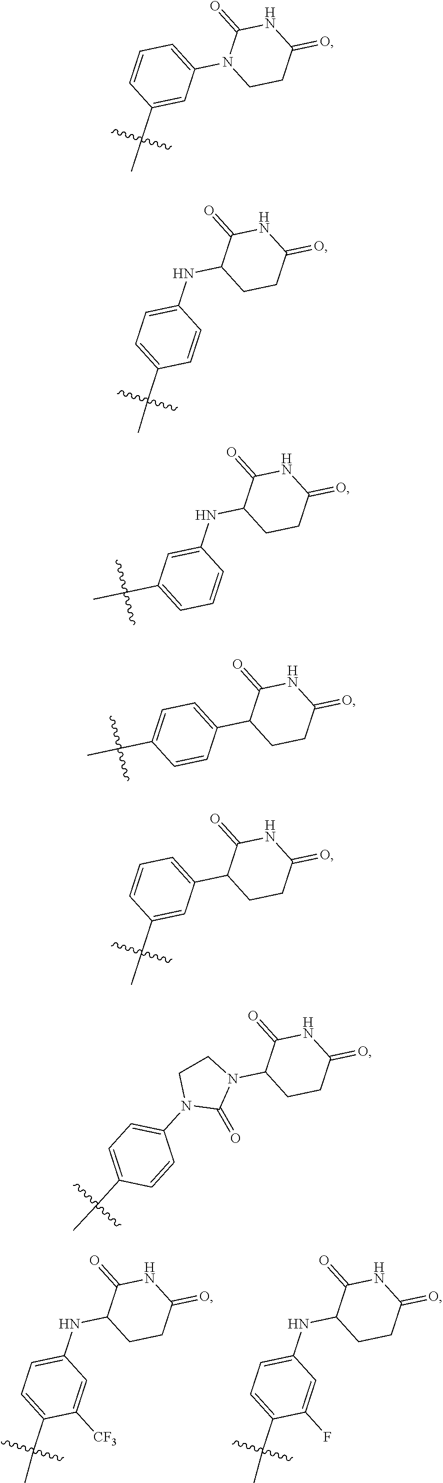

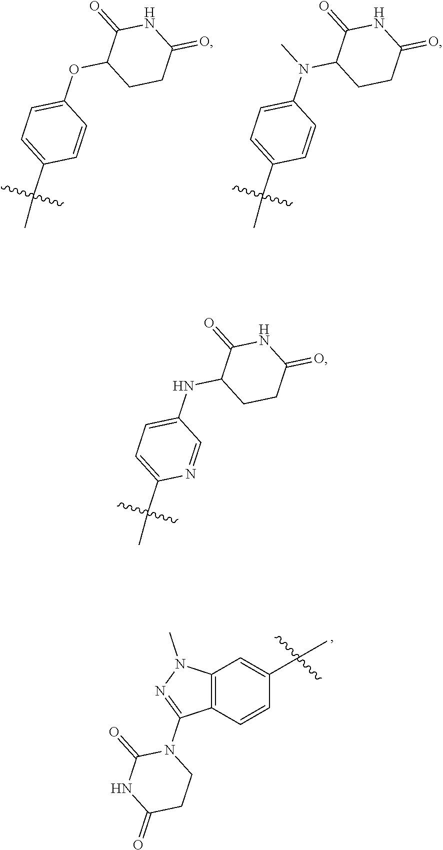 [SMILES: *c1ccc(OC2CCC(=O)NC2=O)cc1 ; *c1ccc(N(C)C2CCC(=O)NC2=O)cc1 ; *c1ccc(NC2CCC(=O)NC2=O)cn1 ; *c1ccc2c(N3CCC(=O)NC3=O)nn(C)c2c1]

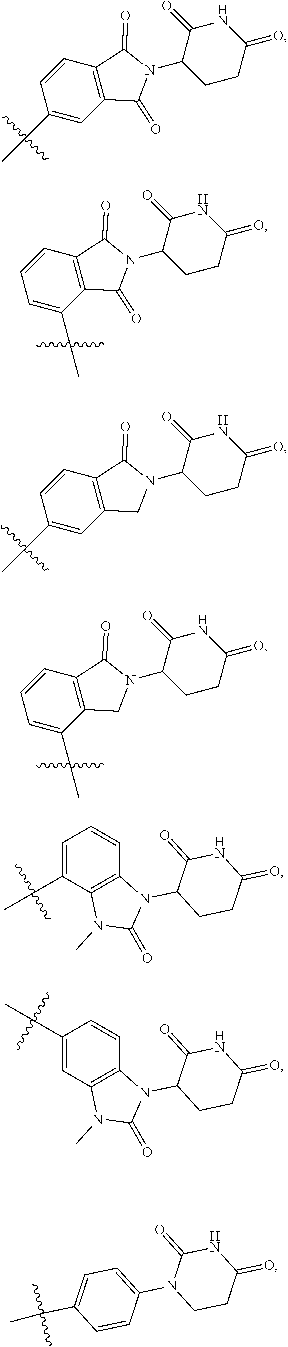

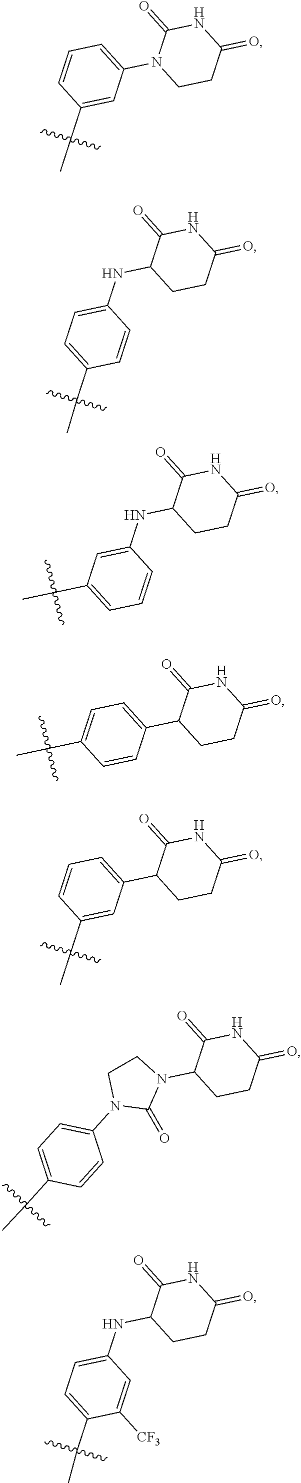

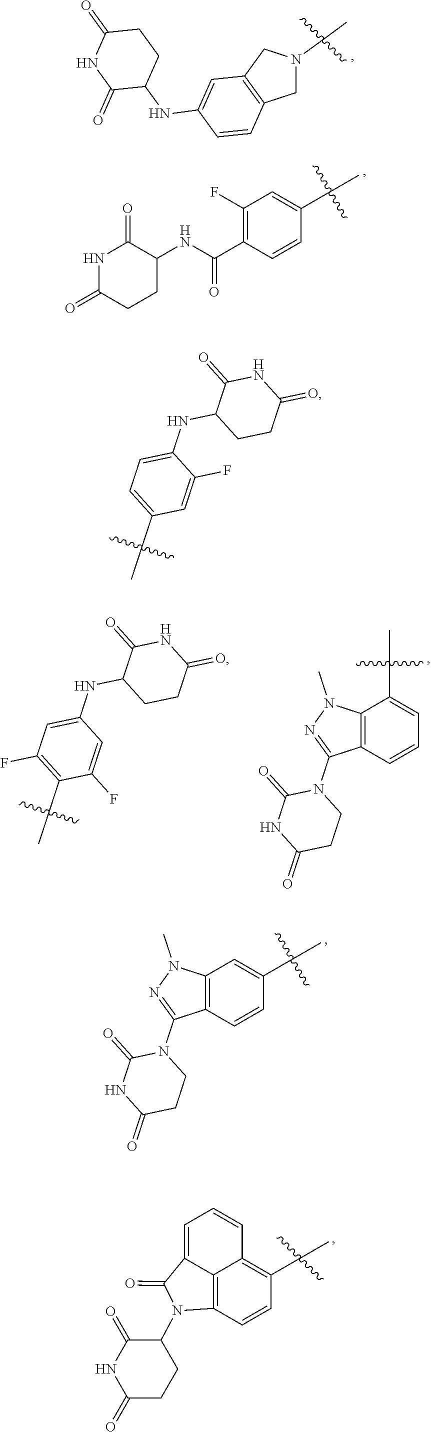

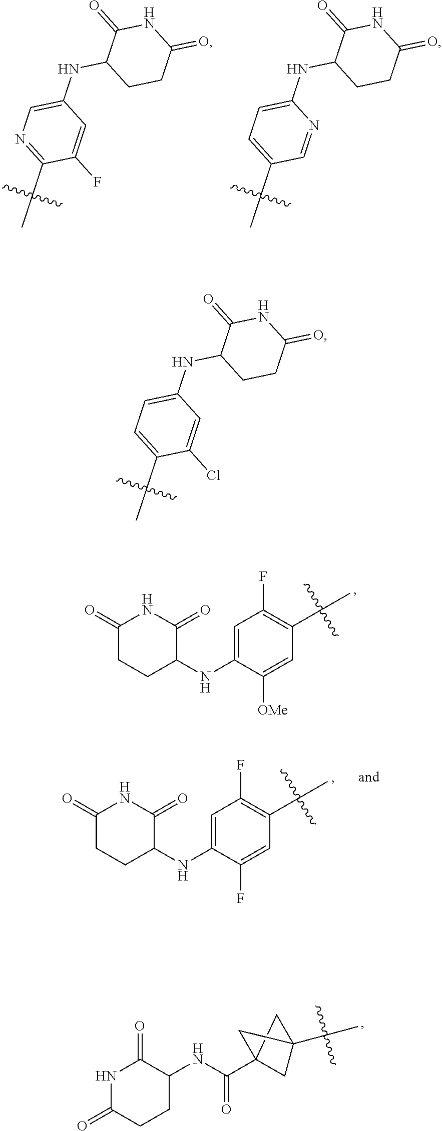 [SMILES: *c1ncc(NC2CCC(=O)NC2=O)cc1F ; *c1ccc(NC2CCC(=O)NC2=O)nc1 ; *c1ccc(NC2CCC(=O)NC2=O)cc1Cl ; *c1cc(OC)c(NC2CCC(=O)NC2=O)cc1F ; *c1cc(F)c(NC2CCC(=O)NC2=O)cc1F ; *C12CC(C(=O)NC3CCC(=O)NC3=O)(C1)C2]

wherein the wavy line represents the point of attachment to group L¹.

In some embodiments, E is selected from the following:

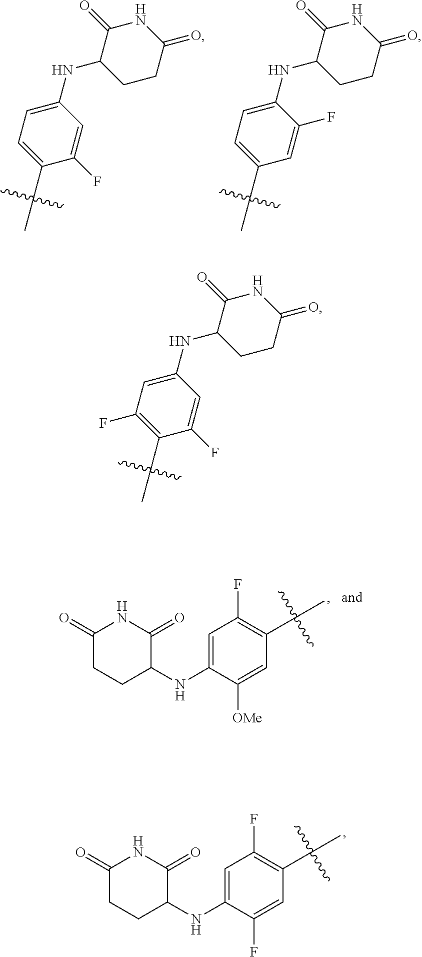 [SMILES: *c1ccc(NC2CCC(=O)NC2=O)cc1F ; *c1ccc(NC2CCC(=O)NC2=O)c(F)c1 ; *c1c(F)cc(NC2CCC(=O)NC2=O)cc1F ; *c1cc(OC)c(NC2CCC(=O)NC2=O)cc1F ; *c1cc(F)c(NC2CCC(=O)NC2=O)cc1F]

wherein the wavy line represents the point of attachment to group L¹.

In some embodiments, E is selected from the following:

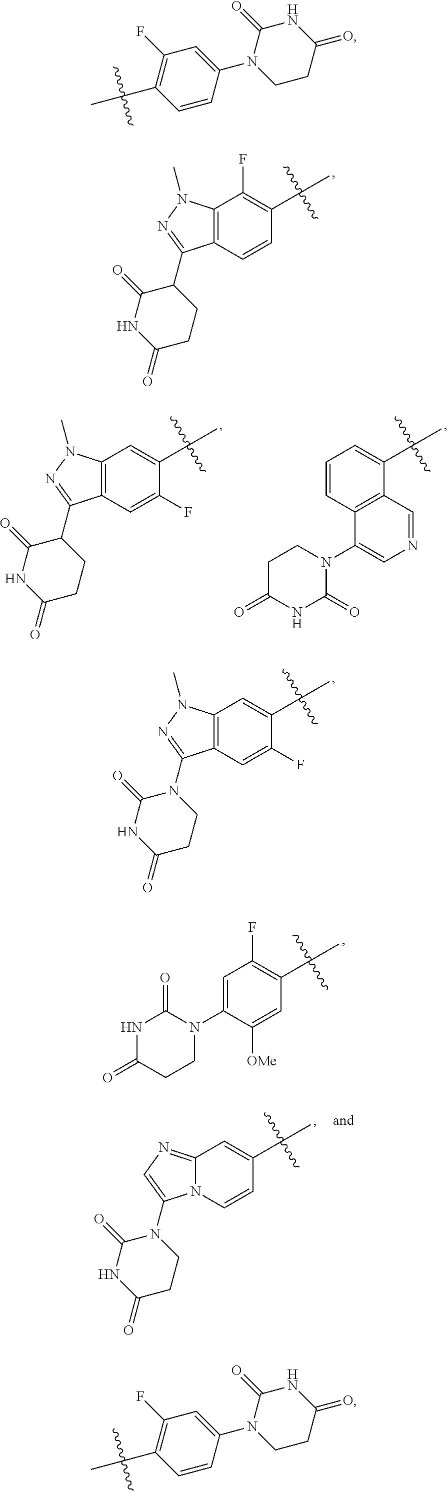

wherein the wavy line represents the point of attachment to group L¹.

In some embodiments, E is selected from the following:

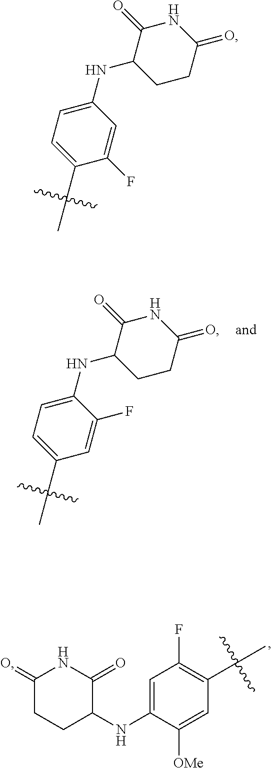 [SMILES: *c1ccc(NC2CCC(=O)NC2=O)cc1F ; *c1ccc(NC2CCC(=O)NC2=O)c(F)c1 ; *c1cc(OC)c(NC2CCC(=O)NC2=O)cc1F]

wherein the wavy line represents the point of attachment to group L¹.

In some embodiments, E is:

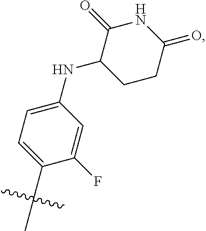 [SMILES: *c1ccc(NC2CCC(=O)NC2=O)cc1F]

wherein the wavy line represents the point of attachment to group L¹.

In some embodiments, E is:

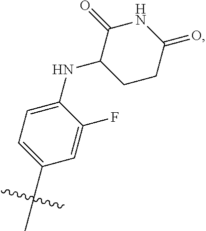 [SMILES: *c1ccc(NC2CCC(=O)NC2=O)c(F)c1]

wherein the wavy line represents the point of attachment to group L¹.

In some embodiments, E is:

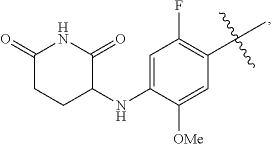 [SMILES: *c1cc(OC)c(NC2CCC(=O)NC2=O)cc1F]

wherein the wavy line represents the point of attachment to group L¹.

In some embodiments, the compound has Formula IA:

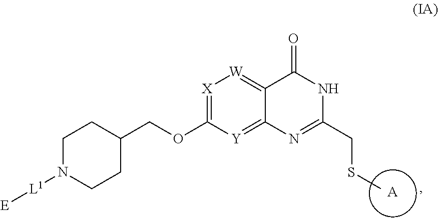

or a pharmaceutically acceptable salt thereof.

In some embodiments, the compound has Formula IA:

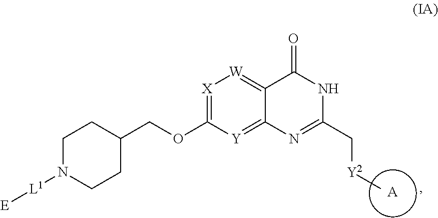

or a pharmaceutically acceptable salt thereof.

In some embodiments, the compound has Formula II:

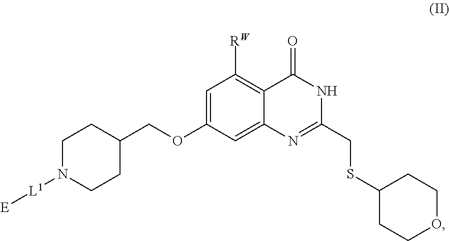

or a pharmaceutically acceptable salt thereof.

In some embodiments, the compound has Formula IIa:

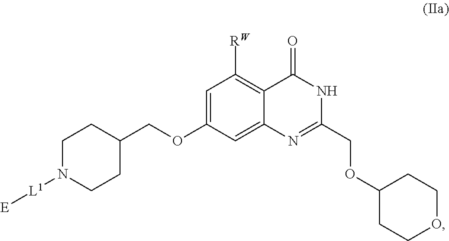

or a pharmaceutically acceptable salt thereof.

In some embodiments, the compound has Formula III:

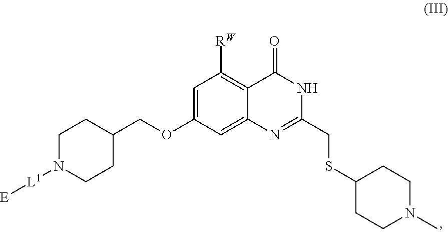

or a pharmaceutically acceptable salt thereof.

In some embodiments, the compound is selected from the following:
N-(6-((2-(2,6-dioxopiperidin-3-yl)-1,3-dioxoisoindolin-4-yl)amino)hexyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(2-((2-(2,6-dioxopiperidin-3-yl)-1,3-dioxoisoindolin-4-yl)amino)ethyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(8-((2-(2,6-dioxopiperidin-3-yl)-1,3-dioxoisoindolin-4-yl)amino)octyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-4-((2-(2-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-3-oxopropoxy)ethoxy)ethyl)amino)isoindoline-1,3-dione;
2-(2,6-dioxopiperidin-3-yl)-4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)-3-oxopropyl)amino)isoindoline-1,3-dione;
2-(2,6-dioxopiperidin-3-yl)-4-((2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)amino)isoindoline-1,3-dione;
N-(2-(2-(2-((2-(2,6-dioxopiperidin-3-yl)-1,3-dioxoisoindolin-4-yl)amino)ethoxy)ethoxy)ethyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(4-((2-(2,6-dioxopiperidin-3-yl)-1,3-dioxoisoindolin-4-yl)amino)butyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-4-((9-(4-(((5-fluoro-4-oxo-2-(((tetrahydro-2H-pyran-4-yl)thio)methyl)-3,4-dihydroquinazolin-7-yl)oxy)methyl)piperidin-1-yl)-9-oxononyl)amino)isoindoline-1,3-dione;
2-(2,6-dioxopiperidin-3-yl)-4-((3-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-3-oxopropoxy)propyl)amino)isoindoline-1,3-dione;
2-(2,6-dioxopiperidin-3-yl)-4-((7-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-oxoheptyl)amino)isoindoline-1,3-dione;
2-(2,6-dioxopiperidin-3-yl)-4-((5-(4-(((5-fluoro-4-oxo-2-(((tetrahydro-2H-pyran-4-yl)thio)methyl)-3,4-dihydroquinazolin-7-yl)oxy)methyl)piperidin-1-yl)-5-oxopentyl)amino)isoindoline-1,3-dione;
N-(2-(3-((2-(2,6-dioxopiperidin-3-yl)-1,3-dioxoisoindolin-4-yl)amino)propoxy)ethyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-5-((2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)amino)isoindoline-1,3-dione;
2-(2,6-dioxopiperidin-3-yl)-5-(4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperazin-1-yl)isoindoline-1,3-dione;
3-((4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)phenyl)amino)piperidine-2,6-dione;
3-(4-(4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperazin-1-yl)-1-oxoisoindolin-2-yl)piperidine-2,6-dione;
3-((4-(1-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperidin-4-yl)phenyl)amino)piperidine-2,6-dione;
1-(4-(1-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperidin-4-yl)phenyl)dihydropyrimidine-2,4(1H,3H)-dione;
2-(2,6-dioxopiperidin-3-yl)-4-(4-((4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperidin-1-yl)methyl)piperidin-1-yl)isoindoline-1,3-dione;
2-(2,6-dioxopiperidin-3-yl)-4-(4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperazin-1-yl)isoindoline-1,3-dione;
2-(2,6-dioxopiperidin-3-yl)-4-((2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethoxy)ethyl)amino)isoindoline-1,3-dione;
2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione;
2-(2,6-dioxopiperidin-3-yl)-4-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione;
N-(2-(2,6-dioxopiperidin-3-yl)-1,3-dioxoisoindolin-5-yl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(2-(2,6-dioxopiperidin-3-yl)-1,3-dioxoisoindolin-4-yl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)isoindoline-1,3-dione;
2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)isoindoline-1,3-dione;
3-(5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)-1-oxoisoindolin-2-yl)piperidine-2,6-dione;
3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)-1-oxoisoindolin-2-yl)piperidine-2,6-dione;
3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)-3-methyl-2-oxo-2,3-dihydro-1H-benzo[d]imidazol-1-yl)piperidine-2,6-dione;
1-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)phenyl)dihydropyrimidine-2,4(1H,3H)-dione;
1-(3-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)phenyl)dihydropyrimidine-2,4(1H,3H)-dione;
2-(2,6-dioxopiperidin-3-yl)-5-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione;
2-(2,6-dioxopiperidin-3-yl)-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione;
2-(2,6-dioxopiperidin-3-yl)-5-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)isoindoline-1,3-dione;
2-(2,6-dioxopiperidin-3-yl)-4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)isoindoline-1,3-dione;
3-(4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)-1-oxoisoindolin-2-yl)piperidine-2,6-dione;
2-(2,6-dioxopiperidin-3-yl)-4-(4-((1-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetyl)piperidin-4-yl)methyl)piperazin-1-yl)isoindoline-1,3-dione;
2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)isoindoline-1,3-dione;
2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)isoindoline-1,3-dione;
5-(3-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)-2-(2,6-dioxopiperidin-3-yl)isoindoline-1,3-dione;
(E)-2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-en-1-yl)isoindoline-1,3-dione;
2-(2,6-dioxopiperidin-3-yl)-5-(4-(((5-fluoro-2-(((1-methylpiperidin-4-yl)thio)methyl)-4-oxo-3,4-dihydroquinazolin-7-yl)oxy)methyl)piperidin-1-yl)isoindoline-1,3-dione;
3-(4-((4-(4-(((5-fluoro-4-oxo-2-(((tetrahydro-2H-pyran-4-yl)thio)methyl)-3,4-dihydroquinazolin-7-yl)oxy)methyl)piperidin-1-yl)but-2-yn-1-yl)amino)phenyl)piperidine-2,6-dione;
3-((4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutyl)methoxy)phenyl)amino)piperidine-2,6-dione;
3-({4-[3-({4-[({5-fluoro-2-[(oxan-4-ylsulfanyl)methyl]-4-oxo-3H-quinazolin-7-yl}oxy)methyl]piperidin-1-yl}methyl)cyclobutoxy]phenyl}amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-3-(trifluoromethyl)phenyl)amino)piperidine-2,6-dione;
3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)phenyl)amino)piperidine-2,6-dione;
3-((4-(3-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)phenyl)amino)piperidine-2,6-dione;
3-((4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)phenyl)amino)piperidine-2,6-dione; and
1-(3-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)phenyl)dihydropyrimidine-2,4(1H,3H)-dione;
   or a pharmaceutically acceptable salt of any of the aforementioned.

In some embodiments, the compound is selected from the following:
3-((4-(4-(2-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(4-(3-(((5-fluoro-4-oxo-2-(((1-(2,2,2-trifluoroethyl)piperidin-4-yl)thio)methyl)-3,4-dihydroquinazolin-7-yl)oxy)methyl)cyclobutyl)piperazin-1-yl)phenyl)amino)piperidine-2,6-dione;
7-(((1s,3s)-3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one;
7-(((1r,3r)-3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one;
5-fluoro-3-(4-methoxybenzyl)-7-((1-(2-(4-nitrophenoxy)ethyl)piperidin-4-yl)methoxy)-2-(((tetrahydro-2H-pyran-4-yl)thio)methyl)quinazolin-4(3H)-one;
(S)-3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
(R)-3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-(5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-oxoisoindolin-2-yl)piperidine-2, 6-dione;
3-(4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-oxoisoindolin-2-yl)piperidine-2,6-dione;
2-(2,6-dioxopiperidin-3-yl)-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)isoindoline-1,3-dione;
2-(2,6-dioxopiperidin-3-yl)-5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)isoindoline-1,3-dione;
3-((3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-(4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenoxy)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)(methyl)amino)piperidine-2,6-dione;
3-((3-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)amino)piperidine-2,6-dione;
3-((4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione;
3-((6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)pyridin-3-yl)amino)piperidine-2,6-dione;
1-(4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)dihydropyrimidine-2,4(1H,3H)-dione;
3-((4-(3-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,6-diazabicyclo[3.1.1]heptan-6-yl)phenyl)amino)piperidine-2,6-dione;
2-(2,6-dioxopiperidin-3-yl)-5-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)ethynyl)isoindoline-1,3-dione;
3-((4-(6-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,6-diazabicyclo[3.1.1]heptan-3-yl)phenyl)amino)piperidine-2,6-dione;
1-(6-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
1-(6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)dihydropyrimidine-2,4(1H,3H)-dione;
3-((4-(((3aR,5r,6aS)-2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)octahydrocyclopenta[c]pyrrol-5-yl)oxy)phenyl)amino)piperidine-2,6-dione;
1-(4-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)dihydropyrimidine-2,4(1H,3H)-dione;
3-(5-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-3-methyl-2-oxo-2,3-dihydro-1H-benzo[d]imidazol-1-yl)piperidine-2,6-dione;
2-(2,6-dioxopiperidin-3-yl)-5-((1r,3r)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
2-(2,6-dioxopiperidin-3-yl)-5-((1s,3s)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
2-(2,6-dioxopiperidin-3-yl)-4-((1r,3r)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
2-(2,6-dioxopiperidin-3-yl)-4-((1s,3s)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutoxy)phenyl)amino)piperidine-2,6-dione;
3-((4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione;
3-((4-(4-(2-(4-(((5-fluoro-2-((((1r,4r)-4-hydroxycyclohexyl)thio)methyl)-4-oxo-3,4-dihydroquinazolin-7-yl)oxy)methyl)piperidin-1-yl)ethyl)piperazin-1-yl)phenyl)amino)piperidine-2,6-dione;
3-((4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)phenyl)amino)piperidine-2,6-dione;
2-(2,6-dioxopiperidin-3-yl)-5-(4-(4-((5-fluoro-4-oxo-2-(((tetrahydro-2H-pyran-4-yl)thio)methyl)-3,4-dihydroquinazolin-7-yl)methyl)piperazin-1-yl)piperidin-1-yl)isoindoline-1,3-dione;
3-((4-(4-(4-(2-(5-fluoro-4-oxo-2-(((tetrahydro-2H-pyran-4-yl)thio)methyl)-3,4-dihydroquinazolin-7-yl)ethyl)piperazin-1-yl)piperidin-1-yl)phenyl)amino)piperidine-2,6-dione;
3-((4-((1-(4-(((5-fluoro-4-oxo-2-(((tetrahydro-2H-pyran-4-yl)thio)methyl)-3,4-dihydroquinazolin-7-yl)oxy)methyl)cyclohexyl)piperidin-4-yl)oxy)phenyl)amino)piperidine-2,6-dione;
3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((4-(4-(((5-fluoro-4-oxo-2-(((1-(2,2,2-trifluoroethyl)piperidin-4-yl)thio)methyl)-3,4-dihydroquinazolin-7-yl)oxy)methyl)-[1,4′-bipiperidin]-1′-yl)phenyl)amino)piperidine-2,6-dione; and
3-((4-(3-((4-(((5-fluoro-2-((((1r,4r)-4-hydroxycyclohexyl)thio)methyl)-4-oxo-3,4-dihydroquinazolin-7-yl)oxy)methyl)piperidin-1-yl)methyl)cyclobutoxy)phenyl)amino)piperidine-2,6-dione,
   or a pharmaceutically acceptable salt of any of the aforementioned.

In some embodiments, the compound is selected from:
3-(5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-3-methyl-2-oxo-2,3-dihydro-1H-benzo[d]imidazol-1-yl)piperidine-2,6-dione;
3-((2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)isoindolin-5-yl)amino)piperidine-2,6-dione;
N-(2,6-dioxopiperidin-3-yl)-2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)benzamide;
3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)amino)phenyl)amino)piperidine-2,6-dione;
3-((4-(8-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,8-diazabicyclo[3.2.1]octan-3-yl)phenyl)amino)piperidine-2,6-dione;
3-((4-(4-(2-(4-(((2-(cyclopentylmethyl)-5-fluoro-4-oxo-3,4-dihydroquinazolin-7-yl)oxy)methyl)piperidin-1-yl)ethyl)piperazin-1-yl)phenyl)amino)piperidine-2,6-dione;
3-((2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3,5-difluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione;
1-(7-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)dihydropyrimidine-2,4(1H,3H)-dione;
3-(6-(4-(2-(4-(((5-Fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)piperidine-2,6-dione;
3-(6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-2-oxobenzo[cd]indol-1(2H)-yl)piperidine-2,6-dione;
3-((4-fluoro-3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(4-(2-(4-(((2-(cyclopropylmethyl)-5-fluoro-4-oxo-3,4-dihydroquinazolin-7-yl)oxy)methyl)piperidin-1-yl)ethyl)piperazin-1-yl)-3-fluorophenyl)amino)piperidine-2,6-dione;
3-((4-(4-(2-(4-(((2-(cyclobutylmethyl)-5-fluoro-4-oxo-3,4-dihydroquinazolin-7-yl)oxy)methyl)piperidin-1-yl)ethyl)piperazin-1-yl)-3-fluorophenyl)amino)piperidine-2,6-dione
3-((4-(4-(2-(4-(((2-(cyclopentylmethyl)-5-fluoro-4-oxo-3,4-dihydroquinazolin-7-yl)oxy)methyl)piperidin-1-yl)ethyl)piperazin-1-yl)-3-fluorophenyl)amino)piperidine-2,6-dione;
3-((5-fluoro-6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)pyridin-3-yl)amino)piperidine-2,6-dione;
3-((4-(3-((4-(((5-fluoro-4-oxo-2-(((1-(2,2,2-trifluoroethyl)piperidin-4-yl)thio)methyl)-3,4-dihydroquinazolin-7-yl)oxy)methyl)piperidin-1-yl)methyl)cyclobutoxy)phenyl)amino)piperidine-2,6-dione;
3-((4-(3-((4-(((5-fluoro-4-oxo-2-(2-(tetrahydro-2H-pyran-4-yl)ethyl)-3,4-dihydroquinazolin-7-yl)oxy)methyl)piperidin-1-yl)methyl)cyclobutoxy)phenyl)amino)piperidine-2,6-dione;
3-((4-((4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclohexyl)oxy)phenyl)amino)piperidine-2,6-dione;
3-((4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione;
3-((4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidin]-1′-yl)phenyl)amino)piperidine-2,6-dione;
3-((4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidin]-1′-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-1-yl)phenyl)amino)piperidine-2,6-dione;
3-((5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)pyridin-2-yl)amino)piperidine-2,6-dione;
3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)oxy)phenyl)amino)piperidine-2,6-dione;
3-((4-(3-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,8-diazabicyclo[3.2.1]octan-8-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-2-(trifluoromethyl)piperazin-1-yl)phenyl)amino)piperidine-2,6-dione;
3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)(methyl)amino)phenyl)amino)piperidine-2,6-dione;
3-((4-(4-(4-(((5-fluoro-4-oxo-2-(((tetrahydro-2H-pyran-4-yl)thio)methyl)-3,4-dihydroquinazolin-7-yl)oxy)methyl)cyclohexyl)piperazin-1-yl)phenyl)amino)piperidine-2,6-dione;
3-((4-(4-(4-(2-((5-fluoro-4-oxo-2-(((tetrahydro-2H-pyran-4-yl)thio)methyl)-3,4-dihydroquinazolin-7-yl)oxy)ethyl)piperazin-1-yl)piperidin-1-yl)phenyl)amino)piperidine-2,6-dione;
3-((3-fluoro-4-(4-(1-(2-((5-fluoro-4-oxo-2-(((tetrahydro-2H-pyran-4-yl)thio)methyl)-3,4-dihydroquinazolin-7-yl)oxy)ethyl)piperidin-4-yl)piperazin-1-yl)phenyl)amino)piperidine-2,6-dione;
3-((4-(3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidin-1-yl)phenyl)amino)piperidine-2,6-dione;
3-((4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-azaspiro[3.5]nonan-7-yl)phenyl)amino)piperidine-2,6-dione;
3-((4-(4-(2-(4-(((5,6-difluoro-4-oxo-2-(((tetrahydro-2H-pyran-4-yl)thio)methyl)-3,4-dihydroquinazolin-7-yl)amino)methyl)piperidin-1-yl)ethyl)piperazin-1-yl)phenyl)amino)piperidine-2,6-dione;
3-((4-(3-(4-(2-((5-fluoro-4-oxo-2-(((tetrahydro-2H-pyran-4-yl)thio)methyl)-3,4-dihydroquinazolin-7-yl)oxy)ethyl)piperazin-1-yl)azetidin-1-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl-2,2,3,3,5,5,6,6-d8)phenyl)amino)piperidine-2,6-dione;
3-((4-(5-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)pyridin-2-yl)phenyl)amino)piperidine-2,6-dione;
3-((4-(6-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)pyridin-3-yl)phenyl)amino)piperidine-2,6-dione;
N-(2,6-dioxopiperidin-3-yl)-3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)bicyclo[1.1.1]pentane-1-carboxamide;
3-((3-fluoro-4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione;
3-((3-chloro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(7-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-azaspiro[3.5]nonan-2-yl)phenyl)amino)piperidine-2,6-dione;
3-((5-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-2-methoxyphenyl)amino)piperidine-2,6-dione;
3-((5-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-2-methoxyphenyl)amino)piperidine-2,6-dione;
3-((2,5-difluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3-chloro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-azaspiro[3.5]nonan-7-yl)phenyl)amino)piperidine-2,6-dione;
3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)phenyl)amino)piperidine-2,6-dione;
3-((3-fluoro-4-(3-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)azetidin-1-yl)phenyl)amino)piperidine-2,6-dione;
3-((3-fluoro-4-(4-(3-(4-(((5-fluoro-4-oxo-2-(2-(tetrahydro-2H-pyran-4-yl)ethyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)(methyl)amino)phenyl)amino)piperidine-2,6-dione;
3-((3-fluoro-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-[1,3′-biazetidin]-1′-yl)phenyl)amino)piperidine-2,6-dione; and
N-(2,6-dioxopiperidin-3-yl)-2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)benzamide,
or a pharmaceutically acceptable salt of any of the aforementioned.

In some embodiments, the compound is selected from the following:
3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)amino)phenyl)amino)piperidine-2,6-dione;
N-(2,6-dioxopiperidin-3-yl)-2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzamide;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carbonyl)piperidin-1-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperidin-1-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperazin-1-yl)phenyl)amino)piperidine-2,6-dione;
1-(6-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
3-(6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-1-methyl-1H-indazol-3-yl)piperidine-2,6-dione;
1-(3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)dihydropyrimidine-2,4(1H,3H)-dione;
1-(3-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)dihydropyrimidine-2,4(1H,3H)-dione;
1-(7-fluoro-6-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
1-(5-fluoro-6-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
1-(6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-1-methyl-1H-indazol-3-yl)dihydropyrimidine-2,4(1H,3H)-dione;
1-(8-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)isoquinolin-4-yl)dihydropyrimidine-2,4(1H,3H)-dione;
1-(5-fluoro-6-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
1-(5-fluoro-6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-1-methyl-1H-indazol-3-yl)dihydropyrimidine-2,4(1H,3H)-dione;
1-(4-fluoro-3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)dihydropyrimidine-2,4(1H,3H)-dione;
1-(5-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-2-methoxyphenyl)dihydropyrimidine-2,4(1H,3H)-dione;
1-(7-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)imidazo[1,2-a]pyridin-3-yl)dihydropyrimidine-2,4(1H,3H)-dione;
1-(7-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)imidazo[1,2-a]pyridin-3-yl)dihydropyrimidine-2,4(1H,3H)-dione;
3-(6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-2-oxobenzo[cd]indol-1(2H)-yl)piperidine-2,6-dione;
3-((2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)-4-hydroxypiperidin-1-yl)phenyl)amino)piperidine-2,6-dione;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)azetidin-1-yl)piperidin-1-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidine]-1′-carbonyl)piperidin-1-yl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)piperazin-1-yl)phenyl)amino)piperidine-2,6-dione;
3-((3-fluoro-4-(4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidine]-1′-carbonyl)piperidin-1-yl)phenyl)amino)piperidine-2,6-dione;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)pyrrolidine-1-carbonyl)piperidin-1-yl)phenyl)amino)piperidine-2,6-dione;
3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidine-1-carbonyl)piperidin-1-yl)phenyl)amino)piperidine-2,6-dione;
3-((4-(4-(3-(4-(((2-(cyclopropylmethyl)-5-fluoro-4-oxo-3,4-dihydroquinazolin-7-yl)oxy)methyl)piperidin-1-yl)azetidine-1-carbonyl)piperidin-1-yl)-3-fluorophenyl)amino)piperidine-2,6-dione;
3-((3-fluoro-4-(4-(3-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)azetidin-1-yl)piperidin-1-yl)phenyl)amino)piperidine-2,6-dione;
1-(3-fluoro-4-(4-((4-(((5-fluoro-4-oxo-2-(((tetrahydro-2H-pyran-4-yl)oxy)methyl)-3,4-dihydroquinazolin-7-yl)oxy)methyl)piperidin-1-yl)methyl)piperidin-1-yl)phenyl)dihydropyrimidine-2,4(1H,3H)-dione;
1-(6-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione;
1-(5-fluoro-6-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione;
1-(5-fluoro-6-(4-((4-(((5-fluoro-4-oxo-2-(((tetrahydro-2H-pyran-4-yl)oxy)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione;
1-(6-(4-(((5-fluoro-4-oxo-2-(((tetrahydro-2H-pyran-4-yl)oxy)methyl)-3,4-dihydroquinazolin-7-yl)oxy)methyl)piperidin-1-yl)-1-methyl-1H-indazol-3-yl)dihydropyrimidine-2,4(1H,3H)-dione;
1-(5-fluoro-6-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)-1-methyl-1H-indazol-3-yl)dihydropyrimidine-2,4(1H,3H)-dione;
1-(6-(1-(2-((5-fluoro-4-oxo-2-(((tetrahydro-2H-pyran-4-yl)thio)methyl)-3,4-dihydroquinazolin-7-yl)oxy)ethyl)piperidin-4-yl)-1-methyl-1H-indazol-3-yl)dihydropyrimidine-2,4(1H,3H)-dione;
1-(6-(1′-(2-((5-fluoro-4-oxo-2-(((tetrahydro-2H-pyran-4-yl)thio)methyl)-3,4-dihydroquinazolin-7-yl)oxy)ethyl)-[1,4′-bipiperidin]-4-yl)-1-methyl-1H-indazol-3-yl)dihydropyrimidine-2,4(1H,3H)-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazine-1-carbonyl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperazin-1-yl)phenyl)amino)piperidine-2,6-dione;
2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)isoindoline-1,3-dione;
2-(2,6-dioxopiperidin-3-yl)-5-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)isoindoline-1,3-dione;
3-((2-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(3-(((5-fluoro-4-oxo-2-(((tetrahydro-2H-pyran-4-yl)thio)methyl)-3,4-dihydroquinazolin-7-yl)oxy)methyl)azetidin-1-yl)ethyl)piperazin-1-yl)phenyl)amino)piperidine-2,6-dione;
3-({4-[4-(2-{4-[({2-[(cyclohexylsulfanyl)methyl]-5-fluoro-4-oxo-3H-quinazolin-7-yl}oxy)methyl]piperidin-1-yl}ethyl)piperazin-1-yl]-3-fluorophenyl}amino)piperidine-2,6-dione;
3-(5-fluoro-6-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-1-methyl-1H-indazol-3-yl)piperidine-2,6-dione;
1-(3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)dihydropyrimidine-2,4(1H,3H)-dione;
1-(3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)dihydropyrimidine-2,4(1H,3H)-dione;
1-(5-fluoro-6-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
(3S)—N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)pyrrolidine-3-carboxamide;
(3R)—N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)pyrrolidine-3-carboxamide;
N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidine-4-carboxamide;
N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)-N-methylpiperidine-4-carboxamide;
1-(7-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
1-(3-fluoro-4-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)phenyl)dihydropyrimidine-2,4(1H,3H)-dione;
1-(7-fluoro-6-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
3-((3-fluoro-4-(4-(2-(4-(((4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
1-(5-fluoro-6-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
1-(3-fluoro-4-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)dihydropyrimidine-2,4(1H,3H)-dione;
1-(7-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)imidazo[1,2-a]pyridin-3-yl)dihydropyrimidine-2,4(1H,3H)-dione;
1-(8-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)isoquinolin-4-yl)dihydropyrimidine-2,4(1H,3H)-dione;
1-(7-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)imidazo[1,2-a]pyridin-3-yl)dihydropyrimidine-2,4(1H,3H)-dione;
1-(5-fluoro-6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)dihydropyrimidine-2,4(1H,3H)-dione;
3-((3-fluoro-4-(4-((1-(2-((5-fluoro-4-oxo-2-(((tetrahydro-2H-pyran-4-yl)thio)methyl)-3,4-dihydroquinazolin-7-yl)oxy)ethyl)piperidin-4-yl)methyl)piperazin-1-yl)phenyl)amino)piperidine-2,6-dione;
3-((3-fluoro-4-(4-(((5-fluoro-4-oxo-2-(((tetrahydro-2H-pyran-4-yl)thio)methyl)-3,4-dihydroquinazolin-7-yl)oxy)methyl)-2-oxo-[1,4′:1′,4″-terpiperidin]-1″-yl)phenyl)amino)piperidine-2,6-dione;
3-((3-fluoro-4-(9-(2-((5-fluoro-4-oxo-2-(((tetrahydro-2H-pyran-4-yl)thio)methyl)-3,4-dihydroquinazolin-7-yl)oxy)ethyl)-3,9-diazaspiro[5.5]undecan-3-yl)phenyl)amino)piperidine-2,6-dione;
3-((2-fluoro-4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)methyl)piperidin-1-yl)phenyl)amino)piperidine-2,6-dione;
3-((2,5-difluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((4-(4-(((2-(cyclopropylmethyl)-5-fluoro-4-oxo-3,4-dihydroquinazolin-7-yl)oxy)methyl)-[1,4′-bipiperidin]-1′-yl)-3-fluorophenyl)amino)piperidine-2,6-dione;
3-((3-fluoro-4-(4-((4-((5-fluoro-4-oxo-2-(((tetrahydro-2H-pyran-4-yl)thio)methyl)-3,4-dihydroquinazolin-7-yl)ethynyl)piperidin-1-yl)methyl)piperidin-1-yl)phenyl)amino)piperidine-2,6-dione;
3-((2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(2-((5-fluoro-4-oxo-2-(((tetrahydro-2H-pyran-4-yl)thio)methyl)-3,4-dihydroquinazolin-7-yl)oxy)ethyl)-[1,4′-bipiperidin]-1′-yl)phenyl)amino)piperidine-2,6-dione;
3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)-[1,4′-bipiperidin]-1′-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)-[1,4′-bipiperidin]-1′-yl)phenyl)amino)piperidine-2,6-dione;
3-((3-fluoro-4-(4-(2-(4-((5-fluoro-4-oxo-2-(((tetrahydro-2H-pyran-4-yl)thio)methyl)-3,4-dihydroquinazolin-7-yl)ethynyl)piperidin-1-yl)ethyl)piperazin-1-yl)phenyl)amino)piperidine-2,6-dione;
3-((3-fluoro-4-(6-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)-2-azaspiro[3.3]heptan-2-yl)phenyl)amino)piperidine-2,6-dione;
3-((3-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′:1′,4″-terpiperidin]-1″-yl)phenyl)amino)piperidine-2,6-dione;
3-((5-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-2-methoxyphenyl)amino)piperidine-2,6-dione;
3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-3-(trifluoromethyl)phenyl)amino)piperidine-2,6-dione;
3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)piperidin-1-yl)phenyl)amino)piperidine-2,6-dione;
3-((3-fluoro-4-(4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)piperidin-1-yl)phenyl)amino)piperidine-2,6-dione;
3-((3-fluoro-4-(4-(3-(4-(((4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3-oxopiperazin-1-yl)phenyl)amino)piperidine-2,6-dione;
3-((3-fluoro-4-(4-(3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidin-1-yl)piperidin-1-yl)phenyl)amino)piperidine-2,6-dione;
3-((4-fluoro-3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-({4-[4-(3-{4-[({2-[(cyclohexylsulfanyl)methyl]-5-fluoro-4-oxo-3H-quinazolin-7-yl}oxy)methyl]piperidin-1-yl}azetidin-1-yl)piperidin-1-yl]-3-fluorophenyl}amino)piperidine-2,6-dione,
   or a pharmaceutically acceptable salt of any of the aforementioned.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “pyridinyl,” “pyridyl,” or “a pyridine ring” may refer to a pyridin-2-yl, pyridin-3-yl, or pyridin-4-yl ring.

The term “n-membered,” where “n” is an integer, typically describes the number of ring-forming atoms in a moiety where the number of ring-forming atoms is “n”. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —C(O)NR^{G}— includes both —C(O)NR^{G}— and —NR^{G}C(O)— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted.

As used herein, the term “substituted” means that a hydrogen atom is replaced by a non-hydrogen group. It is to be understood that substitution at a given atom is limited by valency.

As used herein, the term “Cᵢ₋ⱼ,” where i and j are integers, employed in combination with a chemical group, designates a range of the number of carbon atoms in the chemical group with i-j defining the range. For example, C₁₋₆ alkyl refers to an alkyl group having 1, 2, 3, 4, 5, or 6 carbon atoms.

As used herein, the term “alkyl,” employed alone or in combination with other terms, refers to a saturated hydrocarbon group that may be straight-chain or branched. In some embodiments, the alkyl group contains 1 to 7, 1 to 6, 1 to 4, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, n-heptyl, and the like. In some embodiments, the alkyl group is methyl, ethyl, or propyl. The term “alkylene” refers to a linking alkyl group.

As used herein, “alkenyl,” employed alone or in combination with other terms, refers to an alkyl group having one or more carbon-carbon double bonds. In some embodiments, the alkenyl moiety contains 2 to 6 or 2 to 4 carbon atoms. Example alkenyl groups include, but are not limited to, ethenyl, n-propenyl, isopropenyl, n-butenyl, sec-butenyl, and the like.

As used herein, “alkynyl,” employed alone or in combination with other terms, refers to an alkyl group having one or more carbon-carbon triple bonds. Example alkynyl groups include, but are not limited to, ethynyl, propyn-1-yl, propyn-2-yl, and the like. In some embodiments, the alkynyl moiety contains 2 to 6 or 2 to 4 carbon atoms.

As used herein, “halo” or “halogen”, employed alone or in combination with other terms, includes fluoro, chloro, bromo, and iodo. In some embodiments, halo is F or Cl.

As used herein, the term “haloalkyl,” employed alone or in combination with other terms, refers to an alkyl group having up to the full valency of halogen atom substituents, which may either be the same or different. In some embodiments, the halogen atoms are fluoro atoms. In some embodiments, the alkyl group has 1 to 6 or 1 to 4 carbon atoms. Example haloalkyl groups include CF₃, C₂F5, CHF₂, CCl₃, CHCl₂, C₂Cl₅, and the like.

As used herein, the term “alkoxy,” employed alone or in combination with other terms, refers to a group of formula —O-alkyl. Example alkoxy groups include methoxy, ethoxy, propoxy (e.g., n-propoxy and isopropoxy), t-butoxy, and the like. In some embodiments, the alkyl group has 1 to 6 or 1 to 4 carbon atoms.

As used herein, “haloalkoxy,” employed alone or in combination with other terms, refers to a group of formula —O-(haloalkyl). In some embodiments, the alkyl group has 1 to 6 or 1 to 4 carbon atoms. An example haloalkoxy group is —OCF₃.

As used herein, “amino,” employed alone or in combination with other terms, refers to NH₂.

As used herein, the term “alkylamino,” employed alone or in combination with other terms, refers to a group of formula —NH(alkyl). In some embodiments, the alkylamino group has 1 to 6 or 1 to 4 carbon atoms. Example alkylamino groups include methylamino, ethylamino, propylamino (e.g., n-propylamino and isopropylamino), and the like.

As used herein, the term “dialkylamino,” employed alone or in combination with other terms, refers to a group of formula —N(alkyl)₂. Example dialkylamino groups include dimethylamino, diethylamino, dipropylamino (e.g., di(n-propyl)amino and di(isopropyl)amino), and the like. In some embodiments, each alkyl group independently has 1 to 6 or 1 to 4 carbon atoms.

As used herein, the term “cycloalkyl,” employed alone or in combination with other terms, refers to a non-aromatic cyclic hydrocarbon including cyclized alkyl and alkenyl groups.

Cycloalkyl groups can include mono- or polycyclic (e.g., having 2, 3, or 4 fused, bridged, or spiro rings) ring systems. Also included in the definition of cycloalkyl are moieties that have one or more aromatic rings (e.g., aryl or heteroaryl rings) fused (i.e., having a bond in common with) to the cycloalkyl ring, for example, benzo derivatives of cyclopentane, cyclohexene, cyclohexane, and the like, or pyrido derivatives of cyclopentane or cyclohexane. Ring-forming carbon atoms of a cycloalkyl group can be optionally substituted by oxo. Cycloalkyl groups also include cycloalkylidenes. The term “cycloalkyl” also includes bridgehead cycloalkyl groups (e.g., non-aromatic cyclic hydrocarbon moieties containing at least one bridgehead carbon, such as admantan-1-yl) and spirocycloalkyl groups (e.g., non-aromatic hydrocarbon moieties containing at least two rings fused at a single carbon atom, such as spiro[2.5]octane and the like). In some embodiments, the cycloalkyl group has 3 to 10 ring members, or 3 to 7 ring members. In some embodiments, the cycloalkyl group is monocyclic or bicyclic. In some embodiments, the cycloalkyl group is monocyclic. In some embodiments, the cycloalkyl group is a C₃₋₇ monocyclic cycloalkyl group. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, tetrahydronaphthalenyl, octahydronaphthalenyl, indanyl, and the like. In some embodiments, the cycloalkyl group is cyclopropyl, cyclobutyl, cyclopentyl, or cyclohexyl.

As used herein, the term “cycloalkylalkyl,” employed alone or in combination with other terms, refers to a group of formula cycloalkyl-alkyl-. In some embodiments, the alkyl portion has 1 to 4, 1 to 3, 1 to 2, or 1 carbon atom(s). In some embodiments, the alkyl portion is methylene. In some embodiments, the cycloalkyl portion has 3 to 10 ring members or 3 to 7 ring members. In some embodiments, the cycloalkyl group is monocyclic or bicyclic. In some embodiments, the cycloalkyl portion is monocyclic. In some embodiments, the cycloalkyl portion is a C₃₋₇ monocyclic cycloalkyl group.

As used herein, the term “heterocycloalkyl,” employed alone or in combination with other terms, refers to a non-aromatic ring or ring system, which may optionally contain one or more alkenylene or alkynylene groups as part of the ring structure, which has at least one heteroatom ring member independently selected from nitrogen, sulfur, oxygen, and phosphorus. Heterocycloalkyl groups can include mono- or polycyclic (e.g., having 2, 3 or 4 fused, bridged, or spiro rings) ring systems. In some embodiments, the heterocycloalkyl group is a monocyclic or bicyclic group having 1, 2, 3, or 4 heteroatoms independently selected from nitrogen, sulfur and oxygen. Also included in the definition of heterocycloalkyl are moieties that have one or more aromatic rings (e.g., aryl or heteroaryl rings) fused (i.e., having a bond in common with) to the non-aromatic heterocycloalkyl ring, for example, 1,2,3,4-tetrahydro-quinoline and the like. Heterocycloalkyl groups can also include bridgehead heterocycloalkyl groups (e.g., a heterocycloalkyl moiety containing at least one bridgehead atom, such as azaadmantan-1-yl and the like) and spiroheterocycloalkyl groups (e.g., a heterocycloalkyl moiety containing at least two rings fused at a single atom, such as [1,4-dioxa-8-aza-spiro[4.5]decan-N-yl] and the like). In some embodiments, the heterocycloalkyl group has 3 to 10 ring-forming atoms, 4 to 10 ring-forming atoms, or about 3 to 8 ring forming atoms. In some embodiments, the heterocycloalkyl group has 2 to 20 carbon atoms, 2 to 15 carbon atoms, 2 to 10 carbon atoms, or about 2 to 8 carbon atoms. In some embodiments, the heterocycloalkyl group has 1 to 5 heteroatoms, 1 to 4 heteroatoms, 1 to 3 heteroatoms, or 1 to 2 heteroatoms. The carbon atoms or heteroatoms in the ring(s) of the heterocycloalkyl group can be oxidized to form a carbonyl, an N-oxide, or a sulfonyl group (or other oxidized linkage) or a nitrogen atom can be quaternized. In some embodiments, the heterocycloalkyl portion is a C₂₋₇ monocyclic heterocycloalkyl group. In some embodiments, the heterocycloalkyl group is a morpholine ring, pyrrolidine ring, piperazine ring, piperidine ring, tetrahydropyran ring, tetrahyropyridine, azetidine ring, or tetrahydrofuran ring.

As used herein, the term “heterocycloalkylalkyl,” employed alone or in combination with other terms, refers to a group of formula heterocycloalkyl-alkyl-. In some embodiments, the alkyl portion has 1 to 4, 1 to 3, 1 to 2, or 1 carbon atom(s). In some embodiments, the alkyl portion is methylene. In some embodiments, the heterocycloalkyl portion has 3 to 10 ring members, 4 to 10 ring members, or 3 to 7 ring members. In some embodiments, the heterocycloalkyl group is monocyclic or bicyclic. In some embodiments, the heterocycloalkyl portion is monocyclic. In some embodiments, the heterocycloalkyl portion is a C₂₋₇ monocyclic heterocycloalkyl group.

As used herein, the term “aryl,” employed alone or in combination with other terms, refers to a monocyclic or polycyclic (e.g., a fused ring system) aromatic hydrocarbon moiety, such as, but not limited to, phenyl, 1-naphthyl, 2-naphthyl, and the like. In some embodiments, aryl groups have from 6 to 10 carbon atoms or 6 carbon atoms. In some embodiments, the aryl group is a monocyclic or bicyclic group. In some embodiments, the aryl group is phenyl or naphthyl.

As used herein, the term “arylalkyl,” employed alone or in combination with other terms, refers to a group of formula aryl-alkyl-. In some embodiments, the alkyl portion has 1 to 4, 1 to 3, 1 to 2, or 1 carbon atom(s). In some embodiments, the alkyl portion is methylene. In some embodiments, the aryl portion is phenyl. In some embodiments, the aryl group is a monocyclic or bicyclic group. In some embodiments, the arylalkyl group is benzyl.

As used herein, the term “heteroaryl,” employed alone or in combination with other terms, refers to a monocyclic or polycyclic (e.g., a fused ring system) aromatic hydrocarbon moiety, having one or more heteroatom ring members independently selected from nitrogen, sulfur and oxygen. In some embodiments, the heteroaryl group is a monocyclic or a bicyclic group having 1, 2, 3, or 4 heteroatoms independently selected from nitrogen, sulfur and oxygen. Example heteroaryl groups include, but are not limited to, pyridyl, pyrimidinyl, pyrazinyl, pyridazinyl, triazinyl, furyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, pyrrolyl, azolyl, quinolinyl, isoquinolinyl, benzisoxazolyl, imidazo[1,2-b]thiazolyl or the like. The carbon atoms or heteroatoms in the ring(s) of the heteroaryl group can be oxidized to form a carbonyl, an N-oxide, or a sulfonyl group (or other oxidized linkage) or a nitrogen atom can be quaternized, provided the aromatic nature of the ring is preserved. In some embodiments, the heteroaryl group has from 3 to 10 carbon atoms, from 3 to 8 carbon atoms, from 3 to 5 carbon atoms, from 1 to 5 carbon atoms, or from 5 to 10 carbon atoms. In some embodiments, the heteroaryl group contains 3 to 14, 4 to 12, 4 to 8, 9 to 10, or 5 to 6 ring-forming atoms. In some embodiments, the heteroaryl group has 1 to 4, 1 to 3, or 1 to 2 heteroatoms.

As used herein, the term “heteroarylalkyl,” employed alone or in combination with other terms, refers to a group of formula heteroaryl-alkyl-. In some embodiments, the alkyl portion has 1 to 4, 1 to 3, 1 to 2, or 1 carbon atom(s). In some embodiments, the alkyl portion is methylene. In some embodiments, the heteroaryl portion is a monocyclic or bicyclic group having 1, 2, 3, or 4 heteroatoms independently selected from nitrogen, sulfur and oxygen. In some embodiments, the heteroaryl portion has 5 to 10 carbon atoms.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Cis and trans geometric isomers of the compounds of the present invention may be isolated as a mixture of isomers or as separated isomeric forms.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the invention also include all isotopes of atoms occurring in the intermediates or final compounds. Isotopes include those atoms having the same atomic number but different mass numbers. For example, isotopes of hydrogen include tritium and deuterium. In some embodiments, the compounds of the invention include at least one deuterium atom.

The term “compound,” as used herein, is meant to include all stereoisomers, geometric isomers, tautomers, and isotopes of the structures depicted, unless otherwise specified.

All compounds, and pharmaceutically acceptable salts thereof, can be found together with other substances such as water and solvents (e.g., in the form of hydrates and solvates) or can be isolated.

In some embodiments, the compounds of the invention, or salts thereof, are substantially isolated. By “substantially isolated” is meant that the compound is at least partially or substantially separated from the environment in which it was formed or detected. Partial separation can include, for example, a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof Methods for isolating compounds and their salts are routine in the art.

The term “small molecule PARP14 targeting moiety” refers to a chemical group that binds to PARP14. The small molecule PARP14 targeting moiety can be a group derived from a compound that inhibits the activity of PARP14. In some embodiments, the small molecule PARP14 targeting moiety inhibits the activity of PARP14 with an DC₅₀ of less than 1 μM in an enzymatic assay (see, e.g., Example A).

The term “Ubiquitin Ligase” refers to a family of proteins that facilitate the transfer of ubiquitin to a specific substrate protein, targeting the substrate protein for degradation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid Compounds of the invention can be prepared according to numerous preparatory routes known in the literature. Example synthetic methods for preparing compounds of the invention are provided in the Schemes below.

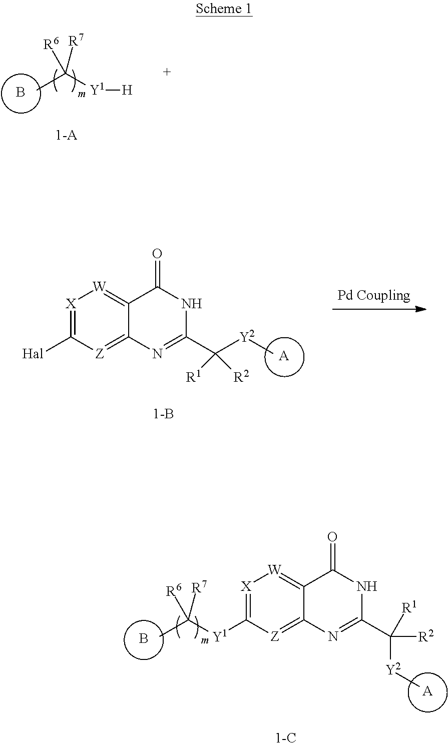

Scheme 1 shows a general synthesis of quinazolinone compounds of the disclosure.

Compounds of formula (1-A), many of which are commercially available or can be made via routes known to one skilled in the art, can be coupled with compounds of formula (1-B), wherein “Hal” is halogen (e.g., Br). Many compounds of formula (1-B) are known in the art and are described in, for example, U.S. Pat. No. 10,562,891. The coupling can be performed under Pd coupling conditions (e.g., in the presence of a Pd reagent such as [Pd(allyl)Cl]2) and provides compounds of formula (1-C).

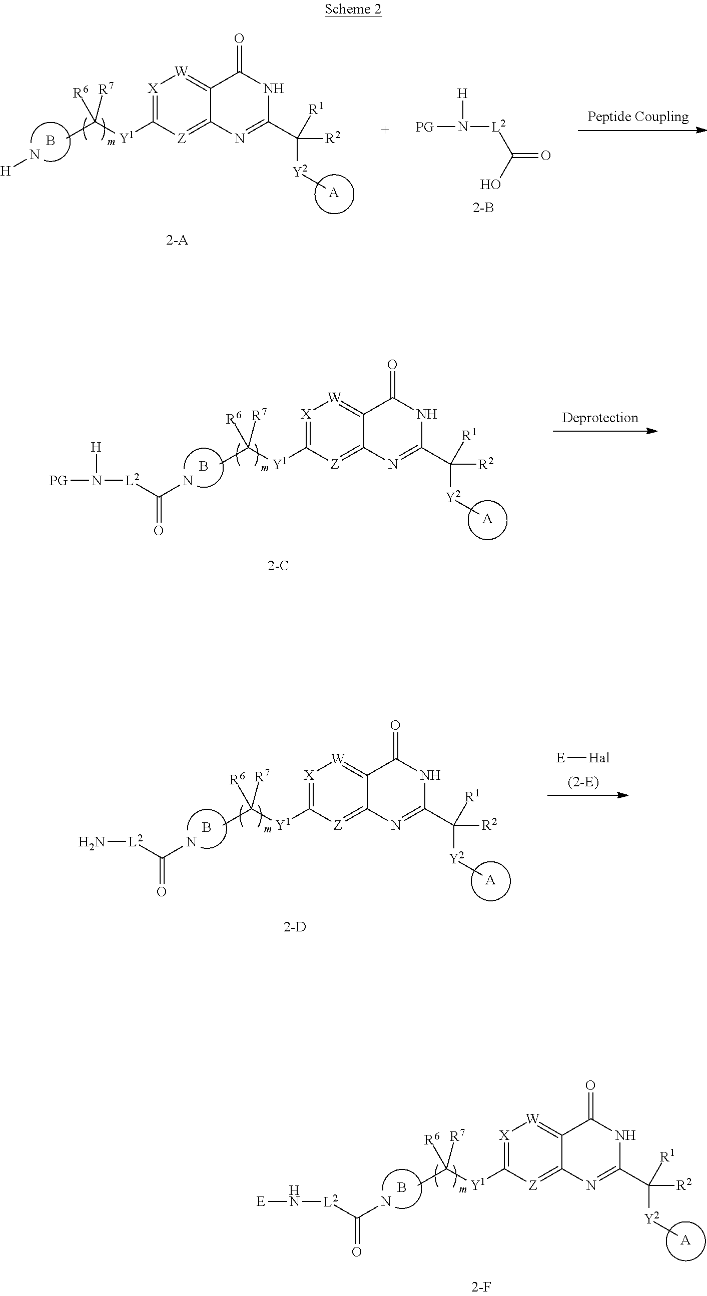

Scheme 2 shows a general synthesis of certain compounds of the invention. Compounds of formula (2-A) can be prepared according to the route provided in Scheme 1 or according to the processes described in, e.g., U.S. Pat. No. 10,562,891. An N-atom of the B-ring of a compound of formula (2-A) can be coupled with a compound of formula (2-B) under peptide coupling conditions (e.g., EDCI, HOBt, and DIPEA; or HATU, DIPEA) to provide a compound of formula (2-C), wherein “PG” refers to a protecting group (e.g., a Boc group). Group L² refers to the internal portion of a linker moiety, Li, as defined herein. Compounds of formula (2-C) can be deprotected (e.g., by treatment with acid) to provide compounds of formula (2-D). Compounds of formula (2-D) can be coupled with compounds of formula (2-E), wherein “Hal” is a halogen atom, to provide compounds of (2-F). Compounds of formula (2-E) are commercially available and are also known in the art. The coupling of compounds of formula (2-D) with compounds of formula (2-E) can be performed, for example, under heating (e.g., 100° C.) in the presence of base (e.g., triethylamine) when “Hal” is F to provide compounds of formula (2-F). The “—NH-L²-C(O)—” group of the compounds of formula (2-F) is equivalent to an Li group as defined herein.

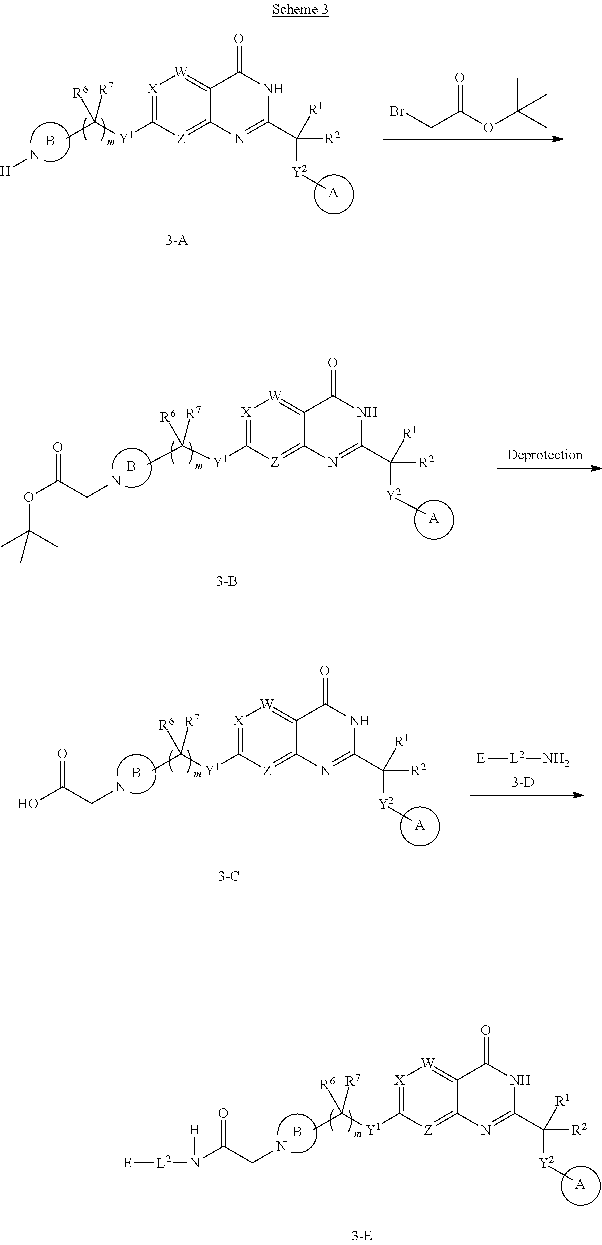

Scheme 3 shows a general synthesis of compounds of the invention. Compounds of formula (3-A), which can be prepared according to Scheme 1, can be treated with tert-butyl 2-bromoacetate to provide compounds of formula (3-B). Compounds of formula (3-B) can be deprotected (e.g., by treatment with an acid) to provide compounds of formula (3-C). Compounds of formula (3-C) can be coupled with compounds of formula (3-D), which are known in the art. The coupling can be performed under peptide coupling conditions (e.g., EDCI, HOBt, and DIPEA; or HATU, DIPEA) to provide compounds of formula (3-E). The “-L²-N(H)—C(O)—CH₂—” group of the compounds of formula (3-E) is equivalent to an Li group as defined herein.

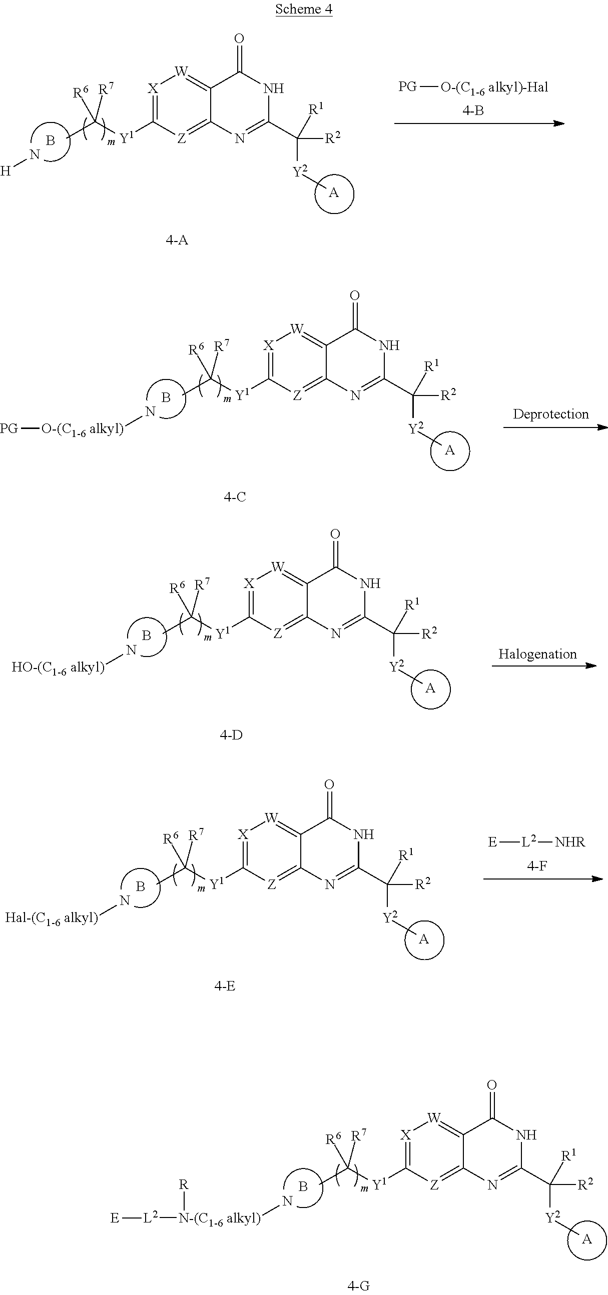

Scheme 4 shows a general synthesis of compounds of the invention. A nucleophilic N-atom of the B-ring of compounds of formula (4-A), which can be prepared according to Scheme 1, can react with an electrophilic compound of formula (4-B), many of which are commercially available and known in the art. The reaction provides compounds of formula (4-C), wherein “PG” refers to a protecting groups (e.g., an alcohol protecting group such as a silyl ether). Compounds of formula (4-C) can be deprotected (e.g., with a fluoride source such as HF) to provide compounds of formula (4-D). Compounds of formula (4-D) can be halogenated (e.g., by treatment with 3-nitrobenzenesulfonyl chloride) to form compounds of formula (4-E), wherein “hal” is a halogen (e.g., Cl). Compounds of formula (4-E) can be reacted with nucleophilic compounds of formula (4-F), many of which are commercially available or known in the art, wherein R is a substituent selected from H, C₁₋₆ alkyl, 4-6 membered heterocycloalkyl, and C₁₋₆ cycloalkyl; to provide compounds of formula (4-G). The “-L¹′-N(H)—(C₁₋₆ alkyl)-” group of the compounds of formula (4-G) is equivalent to an Li group as defined herein.

### Methods of Use

Compounds of the present disclosure can bind to both PARP14 and ubiquitin E3 ligase to cause PARP14 degradation, which is useful in the treatment of various diseases including cancer. In some embodiments, the compounds provided herein can degrade PARP14 in a cell, which comprises contacting the cell with the compound or a pharmaceutically acceptable salt or a stereoisomer thereof. In some embodiments, provided herein is a method for degrading PARP14 in a patient, where the method comprises administering to the patient an effective amount of a compound described herein or a pharmaceutically acceptable salt or a stereoisomer thereof By “degrading PARP14,” it is meant rendering the PARP14 inactive by, for example, altering its structure or breaking down PARP14 into multiple peptide or amino acid fragments.

The compounds of the invention are useful in the treatment of various diseases associated with abnormal expression or activity of PARP14. For example, the compounds of the invention are useful in the treatment of cancer. In some embodiments, the cancers treatable according to the present invention include hematopoietic malignancies such as leukemia and lymphoma. Example lymphomas include Hodgkin's or non-Hodgkin's lymphoma, multiple myeloma, B-cell lymphoma (e.g., diffuse large B-cell lymphoma (DLBCL)), chronic lymphocytic lymphoma (CLL), T-cell lymphoma, hairy cell lymphoma, and Burkett's lymphoma. Example leukemias include acute lymphocytic leukemia (ALL), acute myelogenous leukemia (AML), chronic lymphocytic leukemia (CLL), and chronic myelogenous leukemia (CML).

Other cancers treatable by the administration of the compounds of the invention include liver cancer (e.g., hepatocellular carcinoma), bladder cancer, bone cancer, glioma, breast cancer, cervical cancer, colon cancer, endometrial cancer, epithelial cancer, esophageal cancer, Ewing's sarcoma, pancreatic cancer, gallbladder cancer, gastric cancer, gastrointestinal tumors, head and neck cancer, intestinal cancers, Kaposi's sarcoma, kidney cancer, laryngeal cancer, liver cancer (e.g., hepatocellular carcinoma), lung cancer, prostate cancer, rectal cancer, skin cancer, stomach cancer, testicular cancer, thyroid cancer, and uterine cancer.

In some embodiments, the cancer treatable by administration of the compounds of the invention is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

The compounds of the invention may also have therapeutic utility in PARP14-related disorders in disease areas such as cardiology, virology, neurodegeneration, inflammation, and pain, particularly where the diseases are characterized by overexpression or increased activity of PARP14.

In some embodiments, the compounds of the invention are useful in the treatment of an inflammatory disease. It was found that genetic inactivation of Poly(ADP-Ribose) Polymerase Family Member 14 (PARP14), also referred to as ADP-Ribosyltransferase Diphtheria Toxin-Like 8 (ARTD8) or B Aggressive Lymphoma Protein (BAL2), protected mice against allergen-induced airway disease (Mehrothra et al., J Allergy Clin Immunol, Jul. 25, 2012, 131(2):521-531; and Cho et al., Proc Natl Acad Sci USA, Sep. 20, 2011, 108(38):15972-15977), suppressed the infiltration of immune cells such as eosinophils and neutrophils into the lung, and reduced the release of inflammatory Th2 cytokines. In addition, treatment with a PARP14 inhibitor protected mice in a severe asthma model induced by a sensitization and recall challenge with inhaled Alternaria alternata extract (Eddie et al PMID 35817532). PARP14 inhibitor-treated animals showed a reduced level of airway mucus, blood serum IgE, infiltration of immune cells (eosinophils, neutrophils, and lymphocytes), Th2 cytokines (IL-4, IL-5, and IL13) and alarmins (IL-33 and TSLP) (Eddie et al PMID 35817532 and Ribon internal data).

While not being bound by theory, PARP14 has been shown to affect STAT6 signaling and STAT3 signaling, signaling induced by Th2 cytokines and Th17 cytokines, M1/M2 macrophage polarization, and signaling by lymphocytes. PARP14 has also been shown to be a regulator of Th2/Th17/THF T cell development, involved in B cell development, and involved in eosinophils/neutrophils recruitment/activation. It is believed that the lymphocytes are likely the ILCs (e.g., ILC2 and ILC3) that get activated by the alarmins (e.g., TSLP and IL-33) and are the main producers of the downstream cytokines (e.g., IL-4, IL-5, and IL-13).

It is suggested that PARP14 inhibition affects the asthma phenotype not only at the level of the second order cytokines (e.g., IL-4, IL-5, and IL-13) and the signaling to the myeloid cells, but that PARP14 inhibition also suppresses the alarmins TSLP and IL-33, which are the key upstream drivers of asthma that get released in response to the allergens.

The present invention is directed, inter alia, to a method of treating or preventing an inflammatory disease in a patient comprising administering to the patient a therapeutically effective amount of a compound of the disclosure, or a pharmaceutically acceptable salt thereof. Exemplary inflammatory diseases that are treatable by the disclosed methods include, e.g., asthma, atopic dermatitis, psoriasis, rhinitis, systemic sclerosis, keloids, eosinophilic disorders, pulmonary fibrosis, and other type 2 cytokine pathologies. In some embodiments, the pulmonary fibrosis is idiopathic pulmonary fibrosis.

Additional exemplary inflammatory diseases that are treatable by the disclosed methods include inflammatory bowel diseases (“IBD”), which include ulcerative colitis (“UC” or “colitis”) and Crohn's disease. In some embodiments, the inflammatory disease is inflammatory bowel disease. In some embodiments, the inflammatory disease is ulcerative colitis. In some embodiments, the inflammatory disease is Crohn's disease.

In some embodiments, the inflammatory disease is irritable bowel syndrome.

Eosinophilic disorders that are treatable by the disclosed methods include, e.g., eosinophilic esophagitis (esophagus—EoE), eosinophilic gastritis (stomach—EG), eosinophilic gastroenteritis (stomach and small intestine—EGE), eosinophilic enteritis (small intestine—EE), eosinophilic colitis (large intestine—EC), and eosinophilic chronic rhinosinusitis.

The present invention is further directed, inter alia, to a method of treating or preventing asthma in a patient comprising administering to the patient a therapeutically effective amount of a compound described herein, or a pharmaceutically acceptable salt thereof.

In some embodiments, the asthma is steroid-insensitive asthma, steroid-refractory asthma, steroid-resistant asthma, atopic asthma, nonatopic asthma, persistent asthma, severe asthma, or steroid-refractory severe asthma. In some embodiments, the severe asthma is T2 high endotype, T2 low endotype, or non-T2 endotype. In some embodiments, the severe asthma is T2 high endotype. In some embodiments, the severe asthma is T2 low endotype or non-T2 endotype. In some embodiments, the severe asthma is T2 low endotype. In some embodiments, the severe asthma is non-T2 endotype.

The present invention is further directed, inter alia, to a method of treating or preventing fibrotic diseases such as, but not limited to, pulmonary fibrosis, renal fibrosis, hepatic fibrosis (e.g., NASH and NAFLD), systemic fibrosis, and idiopathic pulmonary fibrosis (IPF). In some embodiments, the fibrotic disease is systemic fibrosis.

The present invention is further directed, inter alia, to a method of treating or preventing chronic obstructive pulmonary disease (COPD), emphysema, and chronic bronchitis.

The present invention is further directed, inter alia, to a method of treating or preventing a skin inflammatory disease such as atopic dermatitis or rosacea.

The present invention further provides a method of:
(a) reducing the level of airway mucus in lung tissue,
(b) reducing blood serum IgE,
(c) reducing immune cell infiltration and activation in bronchoalveolar fluid,
(d) reducing the level of one or more inflammatory cytokines in bronchoalveolar fluid or in lung tissue, or
(e) reducing the level of one or more alarmins in bronchoalveolar fluid or lung tissue,
in a patient, where the method comprises administering to the patient a therapeutically effective amount of a compound disclosed herein, or a pharmaceutically acceptable salt thereof.

In some embodiments, the present invention provides a method of reducing the level of airway mucus in lung tissue in a patient.

In some embodiments, the present invention provides a method of reducing immune cell infiltration and activation in bronchoalveolar fluid in a patient. In some embodiments, the immune cells are eosinophils, neutrophils, or lymphocytes.

In some embodiments, the present invention provides a method of reducing one or more inflammatory cytokines in bronchoalveolar fluid or in lung tissue in a patient. In some embodiments, the inflammatory cytokine is a Th2 cytokine or Th17 cytokine. In some embodiments, the inflammatory cytokine is a Th2 cytokine. In some embodiments, the inflammatory cytokine is IL-4, IL-5, IL13, or IL-17A. In some embodiments, the inflammatory cytokine is IL-4, IL-5, or IL 13.

In some embodiments, the present invention provides a method of reducing an alarmin in bronchoalveolar fluid or in lung tissue in a patient. In some embodiments, the alarmin is IL-25, IL-33 or TSLP.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “contacting” refers to the bringing together of indicated moieties in an in vitro system or an in vivo system. For example, “contacting” PARP14 or “contacting” a cell with a compound of the invention includes the administration of a compound of the present invention to an individual or patient, such as a human, having PARP14, as well as, for example, introducing a compound of the invention into a sample containing a cellular or purified preparation containing PARP14.

As used herein, the term “individual” or “patient,” used interchangeably, refers to mammals, and particularly humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease in an individual who is experiencing or displaying the pathology or symptomatology of the disease (i.e., arresting further development of the pathology and/or symptomatology), or 2) ameliorating the disease in an individual who is experiencing or displaying the pathology or symptomatology of the disease (i.e., reversing the pathology and/or symptomatology).

As used herein the term “preventing” or “prevention” refers to preventing the disease in an individual who may be predisposed to the disease but does not yet experience or display the pathology or symptomatology of the disease.

As used herein, the term “reducing” is with respect to the level in the patient prior to administration. More specifically, when a biomarker or symptom is reduced in a patient, the reduction is with respect to the level of or severity of the biomarker or symptom in the patient prior to administration of the compound of Formula (I), or a pharmaceutically acceptable salt thereof.

### Combination Therapy

One or more additional pharmaceutical agents or treatment methods such as, for example, chemotherapeutics or other anti-cancer agents, immune enhancers, immunosuppressants, immunotherapies, radiation, anti-tumor and anti-viral vaccines, cytokine therapy (e.g., IL2, GM-CSF, etc.), and/or kinase (tyrosine or serine/threonine), epigenetic or signal transduction inhibitors can be used in combination with the compounds of the present invention. The agents can be combined with the present compounds in a single dosage form, or the agents can be administered simultaneously or sequentially as separate dosage forms.

Suitable agents for use in combination with the compounds of the present invention for the treatment of cancer include chemotherapeutic agents, targeted cancer therapies, immunotherapies or radiation therapy. Compounds of this invention may be effective in combination with anti-hormonal agents for treatment of breast cancer and other tumors. Suitable examples are anti-estrogen agents including but not limited to tamoxifen and toremifene, aromatase inhibitors including but not limited to letrozole, anastrozole, and exemestane, adrenocorticosteroids (e.g. prednisone), progestins (e.g. megastrol acetate), and estrogen receptor antagonists (e.g. fulvestrant). Suitable anti-hormone agents used for treatment of prostate and other cancers may also be combined with compounds of the present invention. These include anti-androgens including but not limited to flutamide, bicalutamide, and nilutamide, luteinizing hormone-releasing hormone (LHRH) analogs including leuprolide, goserelin, triptorelin, and histrelin, LHRH antagonists (e.g. degarelix), androgen receptor blockers (e.g. enzalutamide) and agents that inhibit androgen production (e.g. abiraterone).

Angiogenesis inhibitors may be efficacious in some tumors in combination with FGFR inhibitors. These include antibodies against VEGF or VEGFR or kinase inhibitors of VEGFR. Antibodies or other therapeutic proteins against VEGF include bevacizumab and aflibercept. Inhibitors of VEGFR kinases and other anti-angiogenesis inhibitors include but are not limited to sunitinib, sorafenib, axitinib, cediranib, pazopanib, regorafenib, brivanib, and vandetanib Suitable chemotherapeutic or other anti-cancer agents include, for example, alkylating agents (including, without limitation, nitrogen mustards, ethylenimine derivatives, alkyl sulfonates, nitrosoureas and triazenes) such as uracil mustard, chlormethine, cyclophosphamide (Cytoxan™), ifosfamide, melphalan, chlorambucil, pipobroman, triethylene-melamine, triethylenethio-phosphoramine, busulfan, carmustine, lomustine, streptozocin, dacarbazine, and temozolomide.

Other anti-cancer agent(s) include antibody therapeutics to costimulatory molecules such as CTLA-4, 4-1BB, PD-1, and PD-L1, or antibodies to cytokines (IL-10, TGF-β, etc.). Exemplary cancer immunotherapy antibodies include alemtuzumab, ipilimumab, nivolumab, ofatumumab and rituximab.

Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR, e.g., 1996 edition, Medical Economics Company, Montvale, NJ), the disclosure of which is incorporated herein by reference as if set forth in its entirety.

Suitable agents for use in combination with the compounds of the present invention for the treatment of inflammatory diseases include but are not limited to corticosteroids (e.g., prednisone, prednisolone, methylprednisolone, and hydrocortisone); disease-modifying antihreumatic drugs (“DMARDs”, e g, immunosuppressive or anti-inflammatory agents); anti-malarial agents (e.g. hydroxychloroquine and chloroquine); immunosuppressive agents (e.g., cyclophosphamide, azathioprine, mycophenolate mofetil, methotrexate); anti-inflammatory agents (e.g., aspirin, NSAIDs (e.g., ibuprofen, naproxen, indomethacin, nabumetone, celecoxib)); anti-hypertensive agents (e.g., calcium channel blockers (e.g., amlodipine, nifedipine) and diuretics (e.g., furosemide)); statins (e.g., atorvastatin, fluvastatin, lovastatin, pitavastatin, pravastatin, rosuvastatin and simvastatin); anti-B-cell agents (e.g., anti-CD20 (e.g., rituximab), anti-CD22); anti-B-lymphocyte stimulator agents (“anti-BLyS”, e.g., belimumab, blisibimod); type-1 interferon receptor antagonist (e.g., anifrolumab); T-cell modulators (e.g., rigerimod); abatacept; anticoagulants (e.g., heparin, warfarin); and vitamin D supplements.

Additional suitable agents for use in combination of the present invention for the treatment of inflammatory diseases include but not are not limited to sulfonylureas, meglitinides, biguanides, alpha-glucosidase inhibitors, peroxisome proliferators-activated receptor-gamma (i.e., PPAR-gamma) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), anti-platelet agents (for example, aspirin and adenosine diphosphate receptor antagonists that include: clopidogrel, ticlopidine and the like), angiotensin-converting enzyme inhibitors, angiotensin II receptor antagonists and adiponectin.

Suitable agents for use in combination with the compounds of the present invention for the treatment of asthma include but are not limited to beclomethasone (Qvar™), budesonide (Pulmicort Flexhaler™), budesonide/formoterol (Symbicort™), ciclesonide (Alvesco™), flunisolide (Aerospan™), fluticasone (Flovent Diskus™, flovent HFA™, Arnuity Ellipta™), fluticasone/salmeterol (Advair™), mometasone (Asmanex™), mometasone/formoterol (Dulera™), albuterol sulfate (VoSpireER™), formoterol fumarate (Aerolizer™), salmeterol xinafoate (Serevent™), arformoterol tartrate (Brovana™), olodaterol (Striverdi™), fluticasone furoate/vilanterol (Breo Ellipta™), fluticasone furoate/umeclidinium/vilanterol (Trelegy Ellipta™), fluticasone propionate/salmeterol (AirDuo™), glycopyrrolate/formoterol fumarate (Bevespi Aerosphere™), indacaterol/glycopyrrolate (Utibron Neohaler™), tiotropium/olodaterol (Stiolto Respimat™), umeclidinium/vilanterol (Anoro Ellipta™), omalizumab (Xolair™), mepolizumab (NUCALA™), benralizumab (Fasenra™), reslizumab (Cinqair™), dupilumab, tralokinumab, lebrikizumab, etanercept, golimumab brodalumab, and tezepelumab.

### Pharmaceutical Formulations and Dosage Forms

When employed as pharmaceuticals, the compounds of the invention can be administered in the form of pharmaceutical compositions. A pharmaceutical composition refers to a combination of a compound of the invention, or its pharmaceutically acceptable salt, and at least one pharmaceutically acceptable carrier. These compositions can be prepared in a manner well known in the pharmaceutical art, and can be administered by a variety of routes, depending upon whether local or systemic treatment is desired and upon the area to be treated. Administration may be oral, topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, or parenteral.

This invention also includes pharmaceutical compositions which contain, as the active ingredient, one or more of the compounds of the invention above in combination with one or more pharmaceutically acceptable carriers. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The compositions can be formulated in a unit dosage form. The term “unit dosage form” refers to a physically discrete unit suitable as unitary dosages for human subjects and other mammals, each unit containing a predetermined quantity of active material calculated to produce the desired therapeutic effect, in association with a suitable pharmaceutical excipient.

The active compound can be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid pre-formulation composition containing a homogeneous mixture of a compound of the present invention. When referring to these pre-formulation compositions as homogeneous, the active ingredient is typically dispersed evenly throughout the composition so that the composition can be readily subdivided into equally effective unit dosage forms such as tablets, pills and capsules. This solid pre-formulation is then subdivided into unit dosage forms of the type described above containing from, for example, 0.1 to about 500 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration.

The therapeutic dosage of the compounds of the present invention can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compounds of the invention can also be formulated in combination with one or more additional active ingredients which can include any pharmaceutical agent such as anti-viral agents, anti-cancer agents, vaccines, antibodies, immune enhancers, immune suppressants, anti-inflammatory agents and the like.

### EXAMPLES

Equipment: ¹H NMR Spectra were recorded at 300 or 400 MHz using a Bruker AVANCE 300 MHz/400 MHz spectrometer. NMR interpretation was performed using Bruker Topspin software to assign chemical shift and multiplicity. In cases where two adjacent peaks of equal or unequal height were observed, these two peaks may be labeled as either a multiplet or as a doublet. In the case of a doublet, a coupling constant using this software may be assigned. In any given example, one or more protons may not be observed due to obscurity by water and/or solvent peaks. LCMS equipment and conditions are as follows:
1. LC (Basic condition): Shimadzu LC-20ADXR, Binary Pump, Diode Array Detector. Column: Shim-pack scepter C18 33*3.0 mm, 3.0 μm. Mobile phase: A: Water/6.5 mM (NH₄)HCO₃; B: Acetonitrile. Flow Rate: 1.5 mL/min at 40° C. Detector: 190-400 nm. Gradient stop time 2.0 min. Timetable:

| T (min) | A(%) | B(%) |
|---|---|---|
| 0.01 | 90 | 10 |
| 1.20 | 5 | 95 |
| 1.80 | 5 | 95 |
| 1.82 | 90 | 10 |

2. LC (Basic condition): Shimadzu LC-20ADXR, Binary Pump, Diode Array Detector. Column: Shim-pack scepter C18 33*3.0 mm, 3.0 μm. Mobile phase: A: Water/5 mM (NH₄)HCO₃; B: Acetonitrile. Flow Rate: 1.5 mL/min at 40° C. Detector: 190-400 nm. Gradient stop time 2.0 min. Timetable:

| T (min) | A(%) | B(%) |
|---|---|---|
| 0.01 | 90 | 10 |
| 1.20 | 5 | 95 |
| 1.80 | 5 | 95 |
| 1.82 | 90 | 10 |

3. LC (acidic condition): Shimadzu LC-20ADXR, Binary Pump, Diode Array Detector. Column: Halo C18, 30*3.0 mm, 2.0 μm. Mobile phase: A: Water/0.05% TFA, B: Acetonitrile/0.05% TFA. Flow Rate: 1.5 mL/min at 40° C. Detector: 190-400 nm. Gradient stop time, 2.0 min. Timetable:

| T (min) | A(%) | B(%) |
|---|---|---|
| 0.01 | 90 | 5 |
| 1.20 | 5 | 100 |
| 1.80 | 5 | 100 |
| 1.82 | 90 | 5 |

4. LC (Acidic condition): Shimadzu LC-20AD, Binary Pump, Diode Array Detector. Column: Halo C18, 30*3.0 mm, 2.0 μm. Mobile Phase A: Water/0.1% FA; B: Acetonitrile/0.1% FA. Flow Rate:1.5 mL/min at 40° C. Detector: 190-400 nm. Gradient stop time 3.0 min. Timetable:

| T (min) | A(%) | B(%) |
|---|---|---|
| 0.01 | 90 | 5 |
| 1.20 | 5 | 100 |
| 1.80 | 5 | 100 |
| 1.82 | 90 | 5 |

5. The MS detector is configured with electrospray ionization as ionizable source. Acquisition mode: Scan; Nebulizing Gas Flow:1.5 L/min; Drying Gas Flow:15 L/min; Detector Voltage: 0.95-1.25 kv; DL Temperature: 250° C.; Heat Block Temperature: 250° C.; Scan Range: 90.00-900.00 m/z.
6. Sample preparation: samples were dissolved in ACN or methanol at 1˜10 mg/mL, then filtered through a 0.22 μm filter membrane. Injection volume: 1-3 μL.

### Definitions

ACN (acetonitrile); Ac₂O (acetic anhydride); BALF (bronchoalveolar lavage fluid); B₂(OH)₄ (tetrahydroxydiboron); BINAP ((±)-2,2′-bis(diphenylphosphino)-1,1′-binaphthalene); Boc₂O (di-tert-butyl dicarbonate); CaCl₂) (calcium chloride); CH₃CN (acetonitrile); Cs₂CO₃ (cesium carbonate); CsF (cesium fluoride); CuI (copper(I) iodide); Cu(OAc)₂ (copper(II) acetate); DCE (1,2-dichloroethane); DCM or CH₂Cl₂ (dichloromethane); DEAD (diisopropyl azodicarboxylate); DIBAL-H (diisobutylaluminum hydride); DIEA (N,N-diisopropylethylamine); (DiMeIHeptCl)Pd(cinnamyl)Cl₂ ([1,3-Bis[2,6-bis[3-methyl-1-(2-methylpropyl)butyl]phenyl]-4,5-dichloro-1,3-dihydro-2H-imidazol-2-ylidene]chloro[(1,2,3-)-1-phenyl-2-propen-1-yl]-palladium); DMF (N,N-dimethylformamide); DMAP (4-dimethyl aminopyridine); DMPU (1,3-dimethyl-3,4,5,6-tetrahydro-2(1H)-pyrimidinone); DMSO (dimethylsulfoxide); DMSO-d₆ (deuterated dimethylsulfoxide); dtbpy (4,4′-di-tert-butyl-2,2′-dipyridyl); EDCI (1-Ethyl-3-(3-dimethylaminopropyl)carbodiimide); equiv (equivalent); ESI (electrospray ionization); EtOAc (ethyl acetate); EtOH (ethanol); FA (formic acid); Fe (iron); g (gram); h (hour); HATU (1-[bis(dimethylamino)methylene]-1H-1,2,3-triazolo[4,5-b]pyridinium 3-oxid hexafluorophosphate); ¹H NMR (proton nuclear magnetic resonance); HCl (hydrochloric acid); HOAc (acetic acid); HOBT (1-Hydroxybenzotriazole hydrate); Hz (hertz); K₂CO₃ (potassium carbonate); KI (potassium iodide); K₃PO₄ (potassium phosphate tribasic); L (liter); LCMS (liquid chromatography-mass spectrometry); LDA (lithium diisopropylamide); LiAlH₄ (lithium aluminum hydride); LiCl (lithium chloride); LiHMDS (Lithium bis(trimethylsilyl)amide); M (molar); MeCN (acetonitrile); Mel (Iodomethane); MeOH (methanol); mg (milligrams); MHz (megahertz); min (minutes); mL (milliliters), mmol (millimoles); NaBH₄ (sodium borohydride); NaBH₃CN (sodium cyanoborohydride); Na₂CO₃ (sodium carbonate); NaH (sodium hydride); NaHCO₃ (sodium bicarbonate); NaOCN (sodium cyanate); NaOH (sodium hydroxide); Na₂SO₄ (sodium sulfate); Na₂SO₄·10H₂O (sodium sulfate decahydrate); NH₃ (ammonia); NH₄Cl (ammonium chloride); (NH₄)HCO₃ (ammonium bicarbonate); nm (namometers); (NiBr₂·glyme (nickel(II) bromide ethylene glycol dimethyl ether complex); NMI (N-methylimidazole); NMP (N-methylpyrrolidone); PBS (phosphate buffered saline); [Pd(allyl)Cl]₂ (bis(allyl)dichloropalladium); Pd/C (palladium on carbon); Pd₂(dba)₃ (tris(dibenzylideneacetone)dipalladium(0)); Pd₂(dba)₃·CHCl₃ (tris(dibenzylideneacetone) dipalladium-chloroform adduct); Pd(dppf)Cl₂ ([1,1′-bis(diphenylphosphino)ferrocene]dichloropalladium(II)); Pd(OAc)₂ (palladium(II) acetate); Pd PEPPSI IPentCl (dichloro[1,3-bis(2,6-di-3-pentylphenyl)imidazol-2-ylidene](3-chloropyridyl)palladium(II)); Pd(PPh₃)₄(tetrakis(triphenylphosphine) palladium(0)); Pd(PPh₃)₂Cl₂ (trans-dichlorobis(triphenylphosphine)palladium(II)); PE (petroleum ether); PPh₃ (triphenylphosphine); prep-HPLC (preparative high-performance liquid chromatography); ppm (parts per million); RockPhos (di-tert-butyl(2′,4′,6′-triisopropyl-3-methoxy-6-methyl-[1,1′-biphenyl]-2-yl)phosphine); RuPhos (2-Dicyclohexylphosphino-2′,6′-diisopropoxybiphenyl); RuPhos-PdCl-2nd G (chloro(2-dicyclohexylphosphino-2′,6′-diisopropoxy-1,1′-biphenyl)[2-(2′-amino-1,1′-biphenyl)]palladium(II)); RuPhos Pd G3 ((2-Dicyclohexylphosphino-2′,6′-diisopropoxy-1,1′-biphenyl)[2-(2′-amino-1,1′-biphenyl)]palladium(II) methanesulfonate); SEM-Cl (2-(trimethylsilyl)ethoxymethyl chloride); STAB (sodium triacetoxyborohydride); TBAB (tetrabutylammonium bromide); TBAF (tetrabutylammonium fluoride); TBDPSCl (tert-butyldiphenylchlorosilane); t-BuBrettPhos (2-(di-tert-butylphosphino)-2′,4′,6′-triisopropyl-3,6-dimethoxy-1,1′-biphenyl); t-BuOK (potassium tert-butoxide); t-BuONa (sodium tert-butoxide); TCFH (chloro-N,N,N′,N′-tetramethylformamidinium hexafluorophosphate); TEA (triethylamine); TFA (trifluoroacetic acid); TFAA (trifluoroacetic anhydride); TfOH (trifluoromethanesulfonic acid); THF (tetrahydrofuran); T₃P (propylphosphonic anhydride); TsCl (p-toluenesulfonyl chloride); RT (retention time); UV (ultraviolet); and XantPhos (4,5-bis(diphenylphosphino)-9,9-dimethylxanthene).

### Intermediate A1: Synthesis of 2-(2,6-dioxopiperidin-3-yl)-4-(4-(piperidin-4-ylmethyl)piperazin-1-yl)isoindoline-1,3-dione

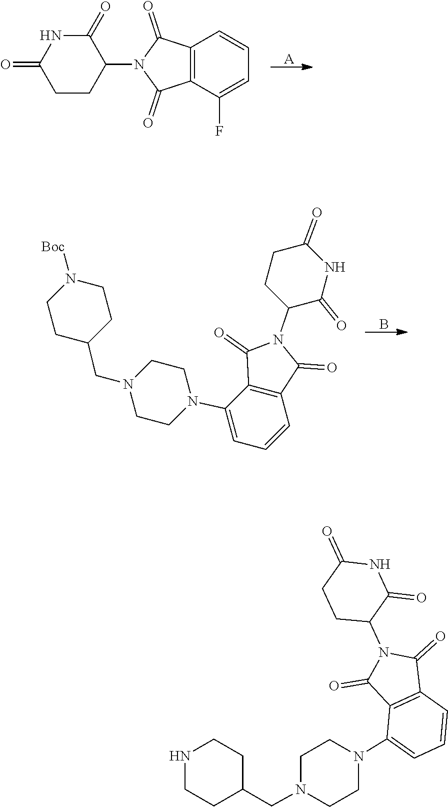

### Step A

A solution of tert-butyl 4-(piperazin-1-ylmethyl)piperidine-1-carboxylate (8.90 g, 31.4 mmol, 1.2 equiv) and 2-(2,6-dioxopiperidin-3-yl)-4-fluoroisoindoline-1,3-dione (7.23 g, 26.2 mmol, 1.0 equiv) and TEA (7.95 g, 78.6 mmol, 3.0 equiv) in NMP (70 mL) was stirred for 3 hours at 70° C. The resulting mixture was diluted with brine (200 mL) and EtOAc (300 mL). The precipitated solids were collected by filtration and washed with EtOAc (30 mL) to afford tert-butyl 4-((4-(2-(2,6-dioxopiperidin-3-yl)-1,3-dioxoisoindolin-4-yl)piperazin-1-yl)methyl)piperidine-1-carboxylate (4.2 g, 30% yield) as a yellow solid. LCMS (ESI, m/z): 540.05 [M+H]⁺.

### Step B

A solution of tert-butyl 4-((4-(2-(2,6-dioxopiperidin-3-yl)-1,3-dioxoisoindolin-4-yl)piperazin-1-yl)methyl)piperidine-1-carboxylate (4.2 g, 7.8 mmol, 1.0 equiv) in trifluoroacetic acid (30 mL) and DCM (90 mL) was stirred for 2 hours at room temperature. The resulting mixture was concentrated under vacuum and then diluted with DCM (60 mL) and water (50 ml). The mixture was neutralized to pH 7 with saturated aqueous Na₂CO₃. The resulting mixture was concentrated under vacuum to remove DCM. The precipitated solids were collected by filtration to afford 2-(2,6-dioxopiperidin-3-yl)-4-(4-(piperidin-4-ylmethyl)piperazin-1-yl)isoindoline-1,3-dione (3.9 g) as a yellow crude solid. The product was used without further purification. LCMS (ESI, m/z): 440.10 [M+H]⁺.

Intermediates A1-a-A1-p were synthesized according to the procedure described for the synthesis 2-(2,6-dioxopiperidin-3-yl)-4-(4-(piperidin-4-ylmethyl)piperazin-1-yl)isoindoline-1,3-dione (Intermediate A1) using appropriate building blocks and modified reaction conditions (such as reagents, reagent ratio, temperature, and reaction time) and purification conditions as needed.

| Intermediate | | |
|---|---|---|
| No. | Structure | Analytical Data |
| A1-a | 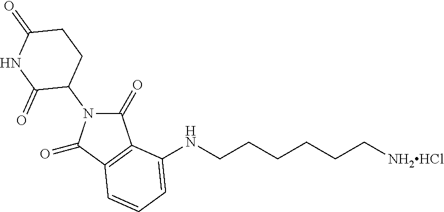 | LCMS (ESI, m/z): 373.15 [M + H]⁺ |
| | 4-((6-aminohexyl)amino)-2-(2,6- | |
| | dioxopiperidin-3-yl)isoindoline-1,3- | |
| | dione hydrochloride | |
| A1-b | 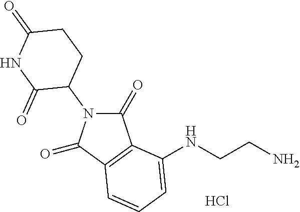 [SMILES: NCCNc1cccc2c1C(=O)N(C1CCC(=O)NC1=O)C2=O ; Cl] | LCMS (ESI, m/z): 317.45 [M + H]⁺ |
| | 4-((2-aminoethyl)amino)-2-(2,6- | |
| | dioxopiperidin-3-yl)isoindoline-1,3- | |
| | dione hydrochloride | |
| A1-c | 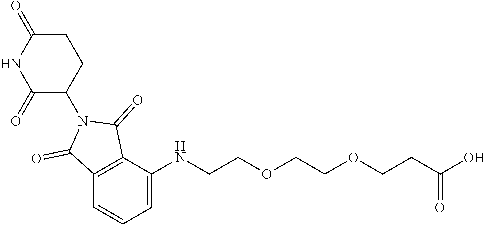 [SMILES: O=C(O)CCOCCOCCNc1cccc2c1C(=O)N(C1CCC(=O)NC1=O)C2=O] | LCMS (ESI, m/z): 434.15 [M + H]⁺ |
| | 3-(2-(2-((2-(2,6-dioxopiperidin-3-yl)- | |
| | 1,3-dioxoisoindolin-4- | |
| | yl)amino)ethoxy)ethoxy)propanoic | |
| | acid | |
| A1-d | 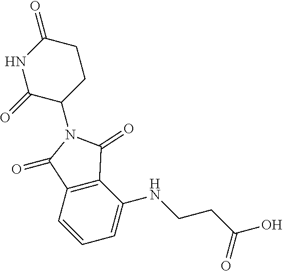 [SMILES: O=C(O)CCNc1cccc2c1C(=O)N(C1CCC(=O)NC1=O)C2=O] | LCMS (ESI, m/z): 346.20 [M + H]⁺ |
| | 3-((2-(2,6-dioxopiperidin-3-yl)-1,3- | |
| | dioxoisoindolin-4-yl)amino)propanoic | |
| | acid | |
| A1-e | 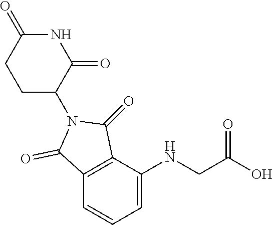 [SMILES: O=C(O)CNc1cccc2c1C(=O)N(C1CCC(=O)NC1=O)C2=O] | LCMS (ESI, m/z): 332.13 [M + H]⁺ |
| | (2-(2,6-dioxopiperidin-3-yl)-1,3- | |
| | dioxoisoindolin-4-yl)glycine | |
| A1-f | 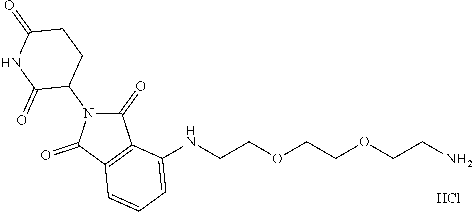 [SMILES: NCCOCCOCCNc1cccc2c1C(=O)N(C1CCC(=O)NC1=O)C2=O ; Cl] | LCMS (ESI, m/z): 405.17 [M + H]⁺ |
| | 4-((2-(2-(2- | |
| | aminoethoxy)ethoxy)ethyl)amino)-2- | |
| | (2,6-dioxopiperidin-3-yl)isoindoline- | |
| | 1,3-dione hydrochloride | |
| A1-g | 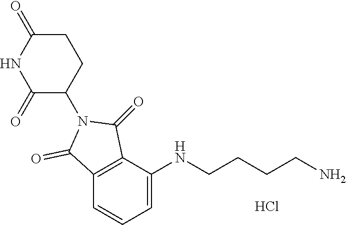 [SMILES: NCCCCNc1cccc2c1C(=O)N(C1CCC(=O)NC1=O)C2=O ; Cl] | LCMS (ESI, m/z): 345.25 [M + H]⁺ |
| | 4-((4-aminobutyl)amino)-2-(2,6- | |
| | dioxopiperidin-3-yl)isoindoline-1,3- | |
| | dione hydrochloride | |
| A1-h | 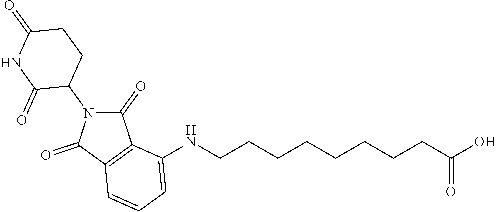 [SMILES: O=C(O)CCCCCCCCNc1cccc2c1C(=O)N(C1CCC(=O)NC1=O)C2=O] | LCMS (ESI, m/z): 430.19 [M + H]⁺ |
| | 9-((2-(2,6-dioxopiperidin-3-yl)-1,3- | |
| | dioxoisoindolin-4-yl)amino)nonanoic | |
| | acid | |
| A1-i | 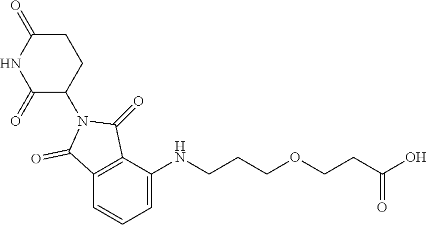 [SMILES: O=C(O)CCOCCCNc1cccc2c1C(=O)N(C1CCC(=O)NC1=O)C2=O] | LCMS (ESI, m/z): 402.15 [M + H]⁺ |
| | 3-(3-((2-,6-dioxopiperidin-3-yl)-1,3- | |
| | dioxoisoindolin-4- | |
| | yl)amino)propoxy)propanoic acid | |
| A1-j | 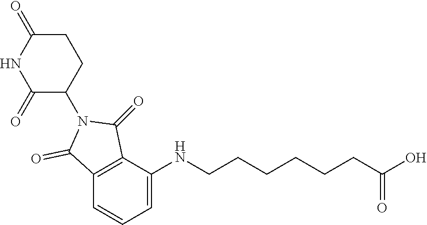 [SMILES: O=C(O)CCCCCCNc1cccc2c1C(=O)N(C1CCC(=O)NC1=O)C2=O] | LCMS (ESI, m/z): 402.19 [M + H]⁺ |
| | 7-((2-(2,6-dioxopiperidin-3-yl)-1,3- | |
| | dioxoisoindolin-4-yl)amino)heptanoic | |
| | acid | |
| A1-k | 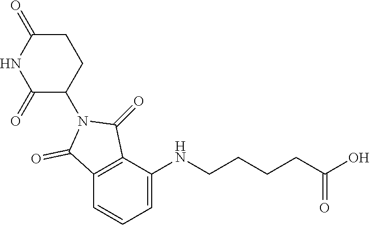 [SMILES: O=C(O)CCCCNc1cccc2c1C(=O)N(C1CCC(=O)NC1=O)C2=O] | LCMS (ESI, m/z): 374.45 [M + H]⁺ |
| | 5-((2-(2,6-dioxopiperidin-3-yl)-1,3- | |
| | dioxoisoindolin-4-yl)amino)pentanoic | |
| | acid | |
| A1-l | 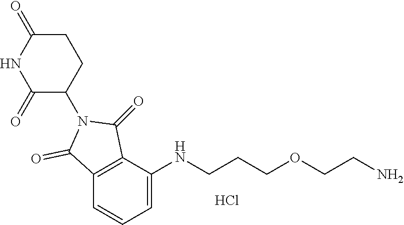 [SMILES: NCCOCCCNc1cccc2c1C(=O)N(C1CCC(=O)NC1=O)C2=O ; Cl] | LCMS (ESI, m/z): 375.20 [M + H]⁺ |
| | 4-((3-(2-aminoethoxy)propyl)amino)- | |
| | 2-(2,6-dioxopiperidin-3- | |
| | yl)isoindoline-1,3-dione hydrochloride | |
| A1-m | 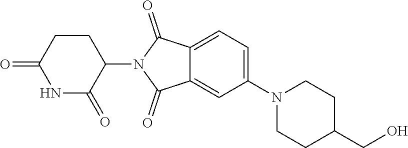 [SMILES: O=C1CCC(N2C(=O)c3ccc(N4CCC(CO)CC4)cc3C2=O)C(=O)N1] | LCMS (ESI, m/z): 372.15 [M + H]⁺ |
| | 2-(2,6-dioxopiperidin-3-yl)-5-(4- | |
| | (hydroxymethyl)piperidin-1- | |
| | yl)isoindoline-1,3-dione | |
| A1-n | 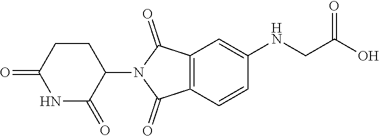 [SMILES: O=C(O)CNc1ccc2c(c1)C(=O)N(C1CCC(=O)NC1=O)C2=O] | LCMS (ESI, m/z): 332.05 [M + H]⁺ |
| | (2-(2,6-dioxopiperidin-3-yl)-1,3- | |
| | dioxoisoindolin-5-yl)glycine | |
| A1-o | 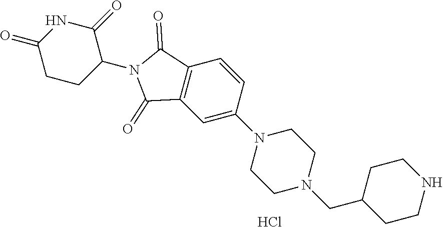 [SMILES: O=C1CCC(N2C(=O)c3ccc(N4CCN(CC5CCNCC5)CC4)cc3C2=O)C(=O)N1 ; Cl] | LCMS (ESI, m/z): 440.20 [M + H]⁺ |
| | 2-(2,6-dioxopiperidin-3-yl)-5-(4- | |
| | (piperidin-4-ylmethyl)piperazin-1- | |
| | yl)isoindoline-1,3-dione hydrochloride | |
| A1-p | 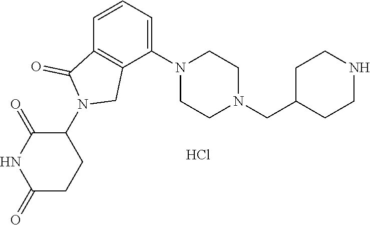 [SMILES: O=C1CCC(N2Cc3c(cccc3N3CCN(CC4CCNCC4)CC3)C2=O)C(=O)N1 ; Cl] | LCMS (ESI, m/z): 426.10 [M + H]⁺ |
| | 3-(1-oxo-4-(4-(piperidin-4- | |
| | ylmethyl)piperazin-1-yl)isoindolin-2- | |
| | yl)piperidine-2,6-dione hydrochloride | |

### Intermediate A2: Synthesis of 2-bromo-N-(2-(2,6-dioxopiperidin-3-yl)-1,3-dioxoisoindolin-4-yl)acetamide

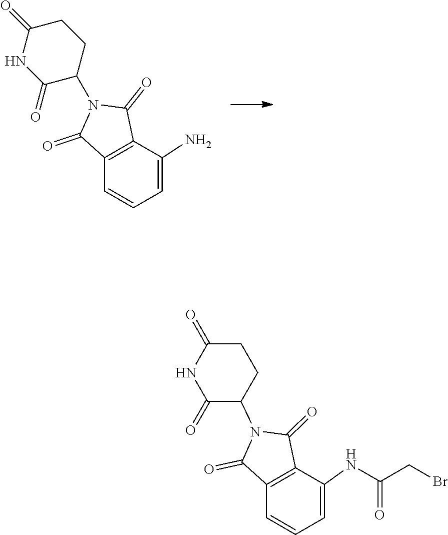 [SMILES: Nc1cccc2c1C(=O)N(C1CCC(=O)NC1=O)C2=O ; O=C1CCC(N2C(=O)c3cccc(NC(=O)CBr)c3C2=O)C(=O)N1]

Bromoacetyl chloride (1.44 g, 9.15 mmol, 2.5 equiv) was added to a solution of pomalidomide (1.0 g, 3.7 mmol, 1.0 equiv) in THF (20 mL) at 0° C. The resulting mixture was stirred for 2 hours at 70° C. and then concentrated under vacuum. The crude product was dissolved in diethyl ether and stirred for 20 min. The precipitated solids were collected by filtration and washed with diethyl ether (3×20 mL) to afford 2-bromo-N-(2-(2,6-dioxopiperidin-3-yl)-1,3-dioxoisoindolin-4-yl)acetamide (1.2 g, 83% yield) as a yellow solid. LCMS (ESI, m/z): 393.95, 395.95 [M+H]⁺.

Intermediate A2-a was synthesized according to the procedure described for the synthesis of 2-bromo-N-(2-(2,6-dioxopiperidin-3-yl)-1,3-dioxoisoindolin-4-yl)acetamide (Intermediate A2) using appropriate building blocks and modified reaction conditions (such as reagents, reagent ratio, temperature, and reaction time) and purification conditions as needed.

| Intermediate | | |
|---|---|---|
| No. | Structure | Analytical Data |
| A2-a | 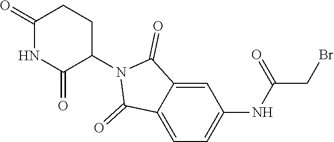 [SMILES: O=C1CCC(N2C(=O)c3ccc(NC(=O)CBr)cc3C2=O)C(=O)N1] | LCMS (ESI, m/z): 394.35 [M + H]⁺ |
| | 2-bromo-N-(2-(2,6-dioxopiperidin-3-yl)- | |
| | 1,3-dioxoisoindolin-5-yl)acetamide | |

### Intermediate A3: Synthesis of 3-((4-(1-(piperidin-4-ylmethyl)piperidin-4-yl)phenyl)amino)piperidine-2,6 dione hydrochloride

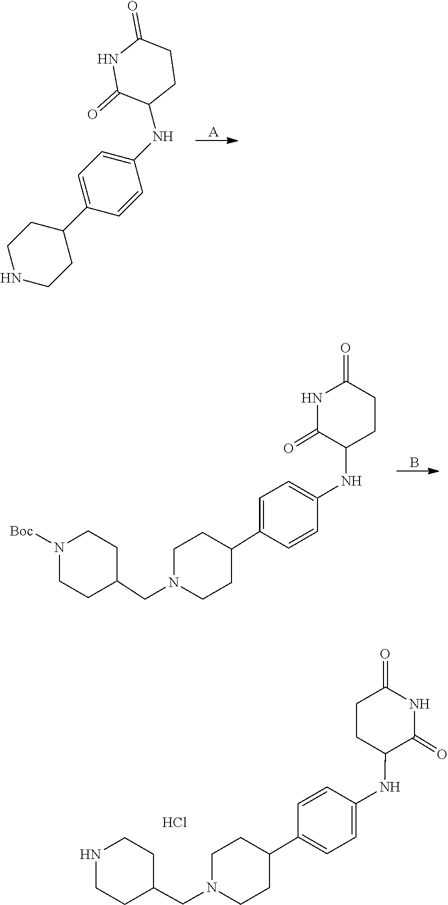

### Step A

To a solution of 3-((4-(piperidin-4-yl)phenyl)amino)piperidine-2,6-dione (600 mg, 2.09 mmol, 1.0 equiv) and tert-butyl 4-formylpiperidine-1-carboxylate (668 mg, 3.13 mmol, 1.5 equiv) in MeOH (20 mL) was added NaBH₃CN (262 mg, 4.18 mmol, 2.0 equiv) portion-wise. The resulting solution was stirred for 4 hours. The mixture was concentrated under reduced pressure and purified directly by silica gel column chromatography, eluting with DCM/MeOH (9:1) to afford tert-butyl 4-((4-(4-((2,6-dioxopiperidin-3-yl)amino)phenyl)piperidin-1-yl)methyl)piperidine-1-carboxylate (639 mg, 63% yield) as a yellow solid. LCMS (ESI, m/z): 485.30 [M+H]⁺.

### Step B

A solution of tert-butyl 4-((4-(4-((2,6-dioxopiperidin-3-yl)amino)phenyl)piperidin-1-yl)methyl)piperidine-1-carboxylate (639 mg, 1.32 mmol, 1.0 equiv) in HCl in 1,4-dioxane (20 mL, 4M) was stirred for 1 hour. The resulting mixture was concentrated under reduced pressure to afford 3-((4-(1-(piperidin-4-ylmethyl)piperidin-4-yl)phenyl)amino)piperidine-2,6-dione hydrochloride (969 mg) as a white crude solid that was used without further purification. LCMS (ESI, m/z): 385.30 [M+H]⁺.

### Intermediate A4: Synthesis of 1-(4-(1-(piperidin-4-ylmethyl)piperidin-4-yl)phenyl)dihydropyrimidine-2,4(1H,3H)-dione

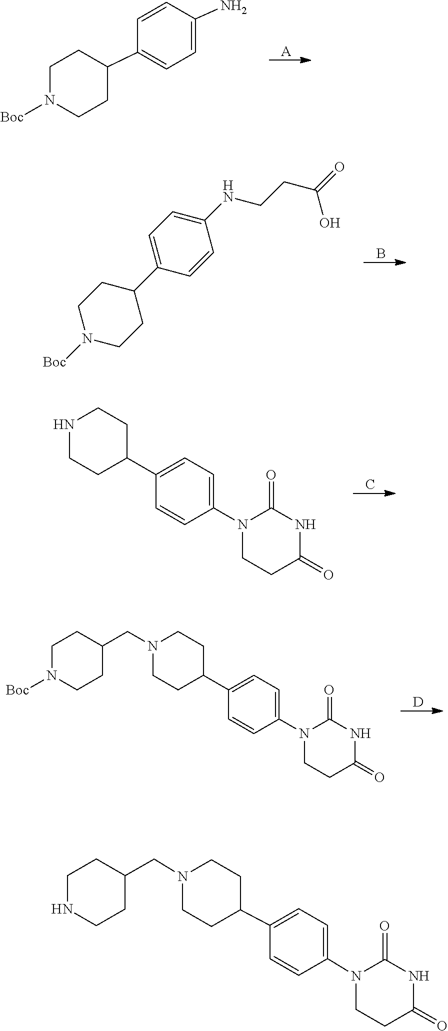

### Step A

A solution of tert-butyl 4-(4-aminophenyl)piperidine-1-carboxylate (500 mg, 1.8 mmol, 1.0 equiv) and acrylic acid (196 mg, 2.71 mmol, 1.5 equiv) in toluene (50 mL) was stirred for 2 h at 120° C. The resulting mixture was concentrated under vacuum. The residue was purified by silica gel column chromatography, eluting with CH₂Cl₂/MeOH (8:1) to afford 3-((4-(1-(tert-butoxycarbonyl)piperidin-4-yl)phenyl)amino)propanoic acid (252 mg, 40% yield) as a yellow solid. LCMS (ESI, m/z): 347.25 [M−H]⁻.

### Step B

A solution of 3-((4-(1-(tert-butoxycarbonyl)piperidin-4-yl)phenyl)amino)propanoic acid (240 mg, 0.69 mmol, 1 equiv) and urea (83 mg, 1.4 mmol, 2 equiv) in HOAc (25 mL) was stirred for 3 hours at 120° C. The mixture was neutralized to pH 7 with saturated aqueous NaHCO₃. The residue was purified by reverse-phase flash chromatography with the following conditions: column, C18 silica gel; mobile phase, MeCN in water (10 mmol/L NH₄HCO₃), 5% to 95% gradient in 20 min; detector, UV 254 nm to afford 1-(4-(piperidin-4-yl)phenyl)dihydropyrimidine-2,4(1H,3H)-dione (95 mg, 50% yield) as a yellow oil. LCMS (ESI, m/z): 274.15 [M+H]⁺.

### Step C

A solution of 1-(4-(piperidin-4-yl)phenyl)dihydropyrimidine-2,4(1H,3H)-dione (85 mg, 0.31 mmol, 1.0 equiv), tert-butyl 4-(bromomethyl)piperidine-1-carboxylate (130 mg, 0.47 mmol, 1.5 equiv) and DIEA (121 mg, 0.93 mmol, 3.0 equiv) in NMP (5 mL) was stirred for 2 hours at 120° C. The resulting mixture was diluted with water (20 mL) and extracted with EtOAc (3×35 mL). The combined organic layers were washed with brine (3×50 mL), dried over anhydrous Na₂SO₄, and filtered. The filtrate was concentrated under reduced pressure and the crude product purified by reversed-phase flash chromatography with the following conditions: column, C18 silica gel; mobile phase, MeCN in water (10 mmol/L NH₄HCO₃), 5% to 95% gradient in 35 min; detector, UV 254 nm to afford tert-butyl 4-((4-(4-(2,4-dioxotetrahydropyrimidin-1(2H)-yl)phenyl)piperidin-1-yl)methyl)piperidine-1-carboxylate (35 mg, 24% yield) as a yellow solid. LCMS (ESI, m/z): 471.35 [M+H]⁺.

### Step D

A solution of tert-butyl 4-((4-(4-(2,4-dioxotetrahydropyrimidin-1(2H)-yl)phenyl)piperidin-1-yl)methyl)piperidine-1-carboxylate (35 mg, 0.074 mmol, 1.0 equiv) and TFA (1 mL) in DCM (1 mL) was stirred for 50 min. The resulting mixture was concentrated under vacuum to afford 1-(4-(1-(piperidin-4-ylmethyl)piperidin-4-yl)phenyl)dihydropyrimidine-2,4(1H,3H)-dione (28 mg) as a yellow crude solid that was used without further purification. LCMS (ESI, m/z): 371.10 [M+H]⁺.

Intermediate A4-a was synthesized according to the procedure described for the synthesis of Synthesis of 1-(4-(1-(piperidin-4-ylmethyl)piperidin-4-yl)phenyl)dihydropyrimidine-2,4(1H,3H)-dione (Intermediate A4) hydrochloride using appropriate building blocks and modified reaction conditions (such as reagents, reagent ratio, temperature, and reaction time) and purification conditions as needed.

| Example No. | Structure | Analytical Data |
|---|---|---|
| A4-a | 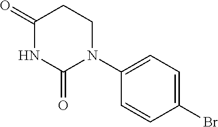 [SMILES: O=C1CCN(c2ccc(Br)cc2)C(=O)N1] | LCMS (ESI, m/z): 270.90 [M + H]⁺ |

Intermediate A4-b was synthesized according to the procedure described for the synthesis of 1-(4-(1-(piperidin-4-ylmethyl)piperidin-4-yl)phenyl)dihydropyrimidine-2,4(1H,3H)-dione hydrochloride (Intermediate A4) using appropriate building blocks and modified reaction conditions (such as reagents, reagent ratio, temperature, and reaction time) and purification conditions as needed.

| Example No. | Structure | Analytical Data |
|---|---|---|
| A4-b | 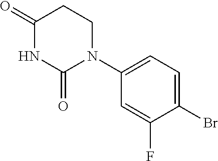 [SMILES: O=C1CCN(c2ccc(Br)c(F)c2)C(=O)N1] | LCMS (ESI, m/z): 286.15 [M + H]⁺ |
| | 1-(4-bromo-3- | |
| | fluorophenyl)dihydropyrimidine- | |
| | 2,4(1H,3H)-dione | |

### Intermediate A5: Synthesis of 3-(1-oxo-5-(piperazin-1-yl)isoindolin-2-yl)piperidine-2,6-dione hydrochloride

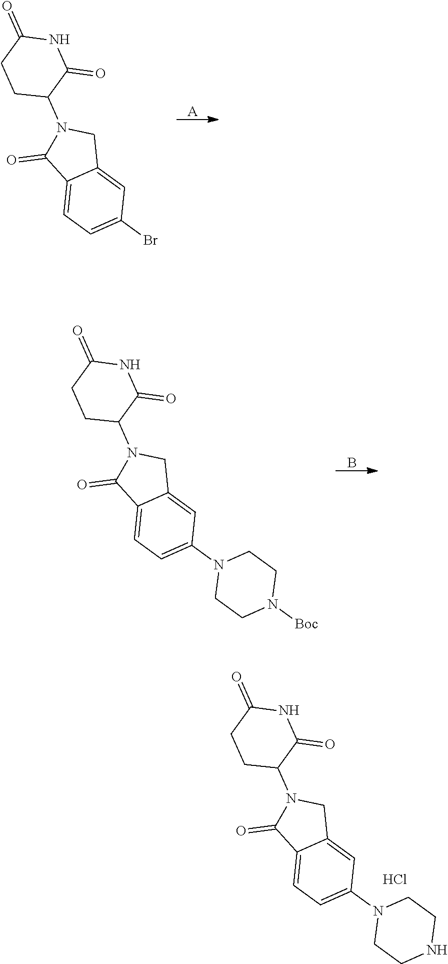

### Step A

A solution of 3-(5-bromo-1-oxoisoindolin-2-yl)piperidine-2, 6-dione (500 mg, 1.55 mmol, 1.0 equiv) and tert-butyl piperazine-1-carboxylate (288 mg, 1.55 mmol, 1.0 equiv), Pd PEPPSI IPentCl (133 mg, 0.16 mmol, 0.1 equiv), Cs₂CO₃ (1008 mg, 3.09 mmol, 2.0 equiv) in 1,4-dioxane (5 mL) was stirred for 1 hour at 100° C. under nitrogen atmosphere. The resulting mixture was concentrated under vacuum. The residue was purified by silica gel column chromatography, eluting with PE/EtOAc (1:1) to afford tert-butyl 4-(2-(2,6-dioxopiperidin-3-yl)-1-oxoisoindolin-5-yl)piperazine-1-carboxylate (400 mg, 60%) as a white solid. LCMS (ESI, m/z): 429.15 [M+H]⁺.

### Step B

A solution of tert-butyl 4-(2-(2,6-dioxopiperidin-3-yl)-1-oxoisoindolin-5-yl)piperazine-1-carboxylate (400 mg, 0.93 mmol, 1.0 equiv) in HCl in 1,4-dioxane (5 mL, 4 M) was stirred for 1 hour. The resulting mixture was concentrated under vacuum to afford 3-(1-oxo-5-(piperazin-1-yl)isoindolin-2-yl)piperidine-2,6-dione hydrochloride (425 mg) as a yellow crude solid. The product was used in the next step without further purification. LCMS (ESI, m/z): 329.10 [M+H]⁺.

Intermediate A5-a to A5-c were synthesized according to the procedure described for the synthesis of 3-(1-oxo-5-(piperazin-1-yl)isoindolin-2-yl)piperidine-2,6-dione hydrochloride (Intermediate A5) using appropriate building blocks and modified reaction conditions (such as reagents, reagent ratio, temperature, and reaction time) and purification conditions as needed.

| Example | | Analytical |
|---|---|---|
| No. | Structure | Data |
| A5-a | 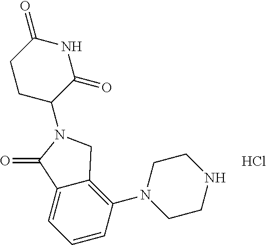 [SMILES: O=C1CCC(N2Cc3c(cccc3N3CCNCC3)C2=O)C(=O)N1 ; Cl] | LCMS (ESI, m/z): 329.10 [M + H]⁺ |
| | 3-(1-oxo-4-(piperazin-1-yl)isoindolin- | |
| | 2-yl)piperidine-2,6-dione | |
| | hydrochloride | |
| A5-b | 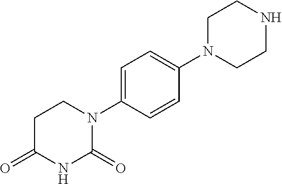 [SMILES: O=C1CCN(c2ccc(N3CCNCC3)cc2)C(=O)N1] | LCMS (ESI, m/z): 275.15 [M + H]⁺ |
| | 1-(4-(piperazin-1- | |
| | yl)phenyl)dihydropyrimidine- | |
| | 2,4(1H,3H)-dione | |
| A5-c | 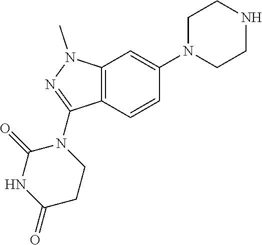 [SMILES: Cn1nc(N2CCC(=O)NC2=O)c2ccc(N3CCNCC3)cc21] | LCMS (ESI, m/z): 329.50 [M + H]⁺ |
| | 1-[1-methyl-6-(piperazin-1-yl)indazol- | |
| | 3-yl]-1,3-diazinane-2,4-dione | |

Intermediates A5-d and A5-e were synthesized according to the procedure described for the synthesis of 3-(1-oxo-5-(piperazin-1-yl)isoindolin-2-yl)piperidine-2,6-dione hydrochloride (Intermediate A5) using appropriate building blocks and modified reaction conditions (such as reagents, reagent ratio, temperature, and reaction time) and purification conditions as needed.

| Example | | Analytical |
|---|---|---|
| No. | Structure | Data |
| A5-d | 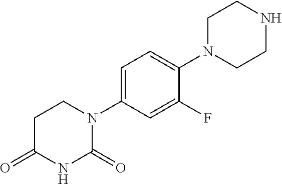 [SMILES: O=C1CCN(c2ccc(N3CCNCC3)c(F)c2)C(=O)N1] | LCMS (ESI, m/z): 292.05 [M + H]⁺ |
| | 1-(3-fluoro-4-(piperazin-1- | |
| | yl)phenyl)dihydropyrimidine- | |
| | 2,4(1H,3H)-dione | |
| A5-e | 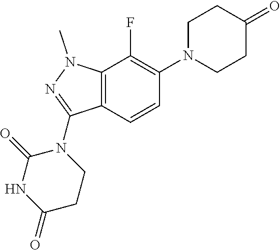 [SMILES: Cn1nc(N2CCC(=O)NC2=O)c2ccc(N3CCC(=O)CC3)c(F)c21] | LCMS (ESI, m/z): 360.15 [M + H]⁺ |
| | 1-(7-fluoro-1-methyl-6-(4- | |
| | oxopiperidin-1-yl)-1H-indazol-3- | |
| | yl)dihydropyrimidine-2,4(1H,3H)- | |
| | dione | |

### Intermediate A6: Synthesis of 2-(2,6-dioxopiperidin-3-yl)-5-(3-(hydroxymethyl)cyclobutoxy)-isoindoline-1,3-dione

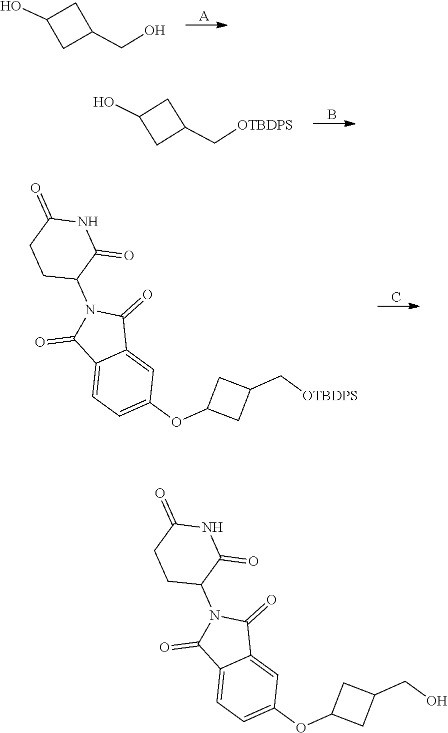

### Step A

To a stirred solution of 3-(hydroxymethyl)cyclobutan-1-ol (2.0 g, 19.6 mmol, 1.0 equiv), TEA (5.94 g, 58.7 mmol, 3.0 equiv) and DMAP (0.24 g, 1.96 mmol, 0.1 equiv) in DCM (5 mL) was added TBDPSCl (5.38 g, 19.6 mmol, 1.0 equiv) dropwise at 0° C. The resulting mixture was stirred for 1 hour at room temperature and then concentrated under vacuum. The residue was purified by silica gel column chromatography, eluting with PE/EtOAc (13/7) to afford 3-(((tert-butyldiphenylsilyl)oxy)methyl)cyclobutan-1-ol (3 g, 45% yield) as a colorless oil. LCMS (ESI, m/z): 341.20 [M+H]⁺.

### Step B

To a stirred solution of 3-(((tert-butyldiphenylsilyl)oxy)methyl)cyclobutan-1-ol (300 mg, 0.88 mmol, 1.0 equiv), 2-(2,6-dioxopiperidin-3-yl)-5-hydroxyisoindole-1,3-dione (362 mg, 1.32 mmol, 1.5 equiv) and PPh₃ (347 mg, 1.32 mmol, 1.5 equiv) in THF (5 mL) was added DEAD (230 mg, 1.32 mmol, 1.5 equiv) dropwise. The resulting mixture was stirred for 4 hours at 50° C. After concentration, the residue was purified by reverse phase flash chromatography with the following conditions: column, C18 silica gel; mobile phase, MeCN in water (0.1% FA), 0% to 80% gradient in 20 min; detector, UV 254 nm. This afforded 5-(3-(((tert-butyldiphenylsilyl)oxy)methyl)cyclobutoxy)-2-(2,6-dioxopiperidin-3-yl)isoindoline-1,3-dione (400 mg, 76% yield) as a white solid. LCMS (ESI, m/z): 597.23[M+H]⁺.

### Step C

To a stirred solution of 5-(3-(((tert-butyldiphenylsilyl)oxy)methyl)cyclobutoxy)-2-(2,6-dioxopiperidin-3-yl)isoindoline-1,3-dione (365 mg, 0.61 mmol, 1.0 equiv) in THF (5 mL) was added TBAF (320 mg, 1.22 mmol, 2.0 equiv) portion-wise. The resulting mixture was stirred for 1 hour. The residue was purified by reverse phase flash chromatography with the following conditions: column, C18 silica gel; mobile phase, MeCN in water (10 mmol/L NH₄HCO₃), 0% to 20% gradient in 20 min; detector, UV 254 nm. This afforded 2-(2,6-dioxopiperidin-3-yl)-5-(3-(hydroxymethyl)cyclobutoxy)isoindoline-1,3-dione (165 mg, 75% yield) as a white solid. LCMS (ESI, m/z): 359.12[M+H]⁺.

Intermediate A6-a was synthesized according to the procedure described for the synthesis of 2-(2,6-dioxopiperidin-3-yl)-5-(3-(hydroxymethyl)cyclobutoxy)-isoindoline-1,3-dione (Intermediate A6) using appropriate building blocks and modified reaction conditions (such as reagents, reagent ratio, temperature, and reaction time) and purification conditions as needed.

| Example | | Analytical |
|---|---|---|
| No. | Structure | Data |
| A6-a | 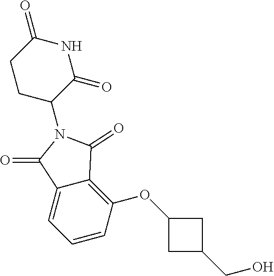 [SMILES: O=C1CCC(N2C(=O)c3cccc(OC4CC(CO)C4)c3C2=O)C(=O)N1] | LCMS (ESI, m/z): 359.15 [M + H]⁺ |
| | 2-(2,6-dioxopiperidin-3-yl)-4-(3- | |
| | (hydroxymethyl)cyclobutoxy)isoindoline- | |
| | 1,3-dione | |

### Intermediate A7: Synthesis of 2-(2,6-dioxopiperidin-3-yl)-4-(piperazin-1-yl)isoindoline-1,3-dione hydrochloride

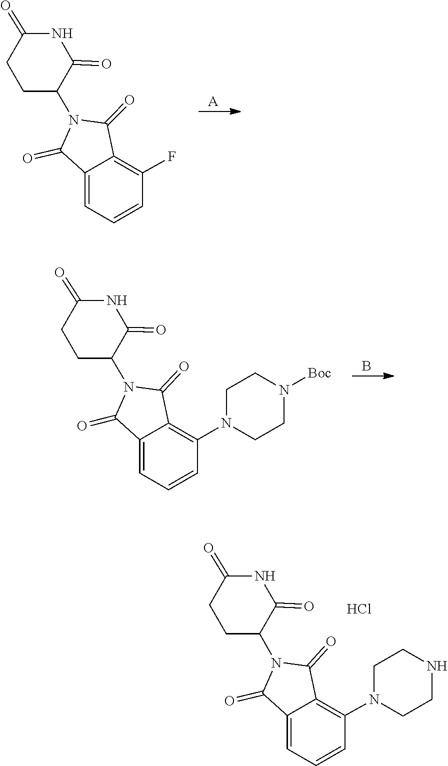

### Step A

A solution of 2-(2,6-dioxopiperidin-3-yl)-4-fluoroisoindole-1,3-dione (1.0 g, 3.62 mmol, 1.0 equiv), tert-butyl piperazine-1-carboxylate (0.74 g, 3.98 mmol, 1.1 equiv) and DIEA (1.40 g, 10.9 mmol, 3.0 equiv) in NMP (5 mL) was stirred for 2 hours at 120° C. The mixture was allowed to cool to room temperature and then diluted with water (25 mL). The precipitated solids were collected by filtration and washed with water (3×10 mL) to afford tert-butyl 4-(2-(2,6-dioxopiperidin-3-yl)-1,3-dioxoisoindolin-4-yl)piperazine-1-carboxylate (1.02 g, 64% yield) as a yellow crude solid. LCMS (ESI, m/z): 443.10 [M+H]⁺.

### Step B

A solution of tert-butyl 4-(2-(2,6-dioxopiperidin-3-yl)-1,3-dioxoisoindolin-4-yl)piperazine-1-carboxylate (1.0 g, 2.26 mmol, 1.0 equiv) in HCl in 1,4-dioxane (50 mL, 4M) was stirred for 50 min. The resulting mixture was concentrated under vacuum to afford 2-(2,6-dioxopiperidin-3-yl)-4-(piperazin-1-yl)isoindoline-1,3-dione hydrochloride (1.1 g) as a yellow crude solid. The crude product was used in next step directly without any further purification. LCMS (ESI, m/z): 343.15 [M+H]⁺.

Intermediates A7-a to A7-d were synthesized according to the procedure described for the synthesis 2-(2,6-dioxopiperidin-3-yl)-4-(piperazin-1-yl)isoindoline-1,3-dione hydrochloride (Intermediate A7) using appropriate building blocks and modified reaction conditions (such as reagents, reagent ratio, temperature, and reaction time) and purification conditions as needed.

| Intermediate | | |
|---|---|---|
| No. | Structure | Analytical Data |
| A7-a | 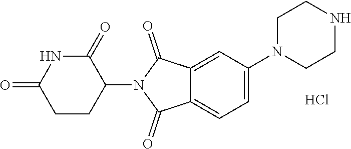 [SMILES: O=C1CCC(N2C(=O)c3ccc(N4CCNCC4)cc3C2=O)C(=O)N1 ; Cl] | LCMS (ESI, m/z): 343.10 [M + H]⁺ |
| | 2-(2,6-dioxopiperidin-3-yl)-5- | |
| | (piperazin-1-yl)isoindoline-1,3-dione | |
| | hydrochloride | |
| A7-b | 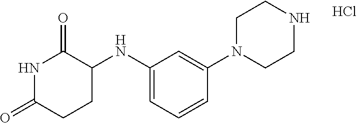 [SMILES: O=C1CCC(Nc2cccc(N3CCNCC3)c2)C(=O)N1 ; Cl] | LCMS (ESI, m/z): 289.10 [M + H]⁺ |
| | 3-((3-(piperazin-1- | |
| | yl)phenyl)amino)piperidine-2,6-dione | |
| | hydrochloride | |
| A7-c | 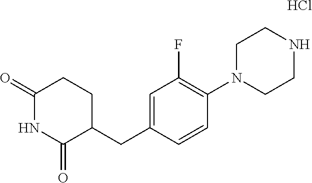 [SMILES: Cl ; O=C1CCC(Cc2ccc(N3CCNCC3)c(F)c2)C(=O)N1] | LCMS (ESI, m/z): 307.10 [M + H]⁺ |
| | 3-((3-fluoro-4-(piperazin-1- | |
| | yl)phenyl)amino)piperidine-2,6-dione | |
| | hydrochloride | |
| A7-d | 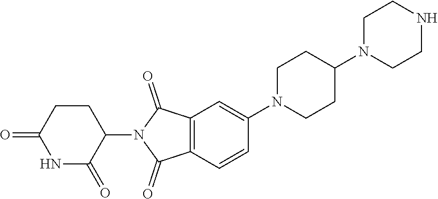 [SMILES: O=C1CCC(N2C(=O)c3ccc(N4CCC(N5CCNCC5)CC4)cc3C2=O)C(=O)N1] | LCMS (ESI, m/z): 426.20 [M + H]⁺ |
| | 2-(2,6-dioxopiperidin-3-yl)-5-(4- | |
| | (piperazin-1-yl)piperidin-1- | |
| | yl)isoindoline-1,3-dione | |

### Intermediate A8: Synthesis of 3-(4-(piperazin-1-yl)phenoxy)piperidine-2,6-dione hydrochloride

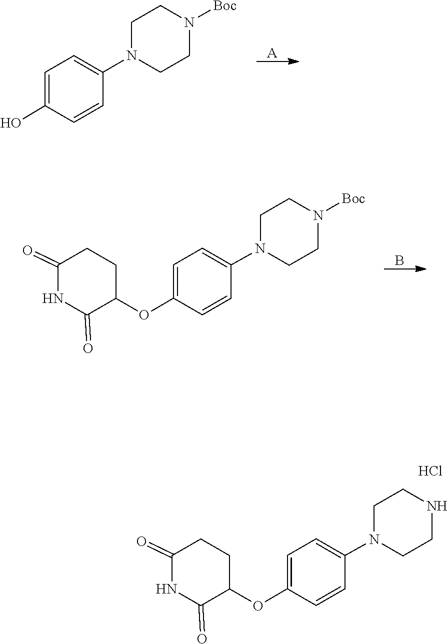

### Step A

To a solution of tert-butyl 4-(4-hydroxyphenyl)piperazine-1-carboxylate (1.0 g, 3.59 mmol, 1.0 equiv) in DMF (2 mL) was added NaH (0.17 g, 7.19 mmol, 2.0 equiv) at 0° C. The resulting mixture was stirred for 15 min at 0° C. 3-bromopiperidine-2,6-dione (0.83 g, 4.31 mmol, 1.2 equiv) was then added, and the mixture was stirred for 12 hours. The reaction was quenched with water at 0° C. and then extracted with EtOAc (3×10 mL). The combined organic layers were washed with brine (3×7 mL), dried over anhydrous Na₂SO₄, and filtered. After filtration, the filtrate was concentrated under reduced pressure. The residue was purified by silica gel column chromatography, eluting with PE/EtOAc (8:1) to afford tert-butyl 4-(4-((2,6-dioxopiperidin-3-yl)oxy)phenyl)piperazine-1-carboxylate (300 mg, 21% yield) as a white solid. LCMS (ESI, m/z): 390.25 [M+H]⁺.

### Step B

A mixture of tert-butyl 4-(4-((2,6-dioxopiperidin-3-yl)oxy)phenyl)piperazine-1-carboxylate (300 mg, 0.77 mmol, 1.0 equiv) in HCl in 1,4-dioxane (5 mL, 4 M) was stirred for 1 hour. The resulting mixture was concentrated under reduced pressure to afford 3-(4-(piperazin-1-yl)phenoxy)piperidine-2,6-dione hydrochloride (250 mg, 99% yield) as a crude yellow solid. The product was used in the next step without further purification. LCMS (ESI, m/z): 290.15 [M+H]⁺.

### Intermediate A9: Synthesis of 3-(methyl(4-(piperazin-1-yl)phenyl)amino)piperidine-2,6-dione hydrochloride

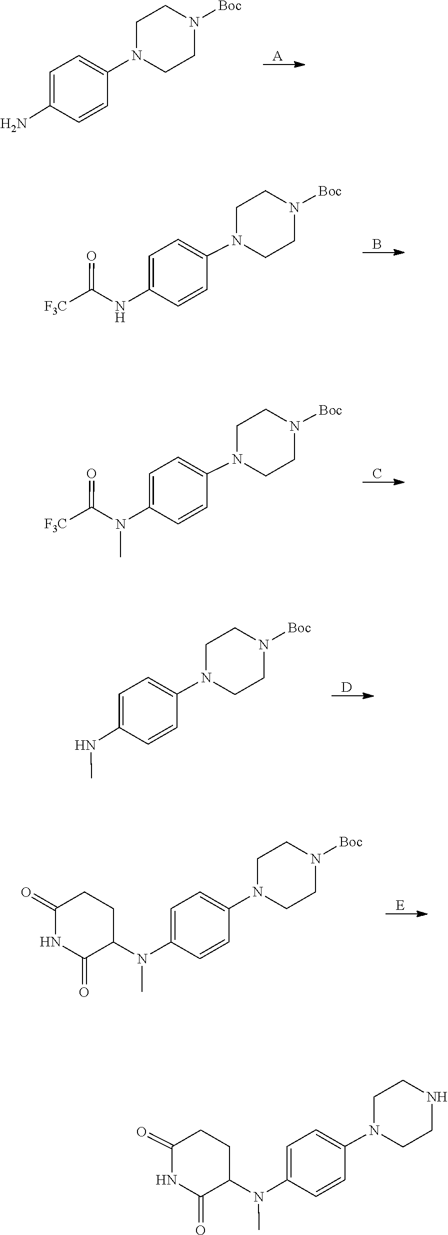

### Step A

A solution of tert-butyl 4-(4-aminophenyl)piperazine-1-carboxylate (3.0 g, 10.8 mmol, 1.0 equiv), TFAA (2.73 g, 13.0 mmol, 1.2 equiv) and TEA (2.19 g, 21.6 mmol, 2.0 equiv) in DCM (20 mL) was stirred for 1 hour. The resulting mixture was concentrated under reduced pressure to afford tert-butyl 4-(4-(2,2,2-trifluoroacetamido)phenyl)piperazine-1-carboxylate (8.3 g) as a brown crude solid. The product was used in the next step without further purification. LCMS (ESI, m/z): 374.15 [M+H]⁺.

### Step B

A solution of tert-butyl 4-(4-(2,2,2-trifluoroacetamido)phenyl)piperazine-1-carboxylate (8.3 g, 22.2 mmol, 1.0 equiv), Mel (3.16 g, 22.2 mmol, 1.0 equiv) and K₂CO₃ (6.14 g, 44.4 mmol, 2.0 equiv) in DMF (10 mL) was stirred overnight at 60° C. The product was precipitated by the addition of water. The solids were collected by filtration. This resulted in tert-butyl 4-(4-(2,2,2-trifluoro-N-methylacetamido)phenyl)piperazine-1-carboxylate (3.6 g, 42% yield) as a black solid. LCMS (ESI, m/z): 388.15 [M+H]⁺.

### Step C

A solution of tert-butyl 4-(4-(2,2,2-trifluoro-N-methylacetamido)phenyl)piperazine-1-carboxylate (3.6 g, 9.29 mmol, 1.0 equiv) and K₂CO₃ (2.57 g, 18.6 mmol, 2.0 equiv) in water (5 mL) and MeOH (20 mL) was stirred for 1 hour. The resulting mixture was concentrated under reduced pressure. The residue was purified by silica gel column chromatography eluting with PE/EtOAc (57:43) to afford tert-butyl 4-(4-(methylamino)phenyl)piperazine-1-carboxylate (2.5 g, 92% yield) as a white solid. LCMS (ESI, m/z): 292.15 [M+H]⁺.

### Step D

A solution of tert-butyl 4-(4-(methylamino)phenyl)piperazine-1-carboxylate (1.5 g, 5.15 mmol, 1.0 equiv), 3-bromopiperidine-2,6-dione (1.19 g, 6.18 mmol, 1.2 equiv) and NaHCO₃ (0.86 g, 10.3 mmol, 2.0 equiv) in ACN (50 mL) was stirred overnight at 90° C. The resulting mixture was concentrated under reduced pressure. The residue was purified by silica gel column chromatography, eluting with PE/EtOAc (30:70) to afford tert-butyl 4-(4-((2,6-dioxopiperidin-3-yl)(methyl)amino)phenyl)piperazine-1-carboxylate (1.6 g, 77% yield) as a light-yellow powder. LCMS (ESI, m/z): 403.15 [M+H]⁺.

### Step E

A solution of tert-butyl 4-(4-((2,6-dioxopiperidin-3-yl)(methyl)amino)phenyl)piperazine-1-carboxylate (1.6 g, 3.98 mmol, 1.0 equiv) in HCl in 1,4-dioxane (30 mL, 4M) was stirred for 50 min. The resulting mixture was concentrated under reduced pressure to afford 3-(methyl(4-(piperazin-1-yl)phenyl)amino)piperidine-2,6-dione hydrochloride (2.1 g) as a light-green solid. The product was used in the next step without further purification. LCMS (ESI, m/z): 303.15 [M+H]⁺.

### Intermediate A10: Synthesis of 3-((3-(piperidin-4-yl)phenyl)amino)piperidine-2,6-dione

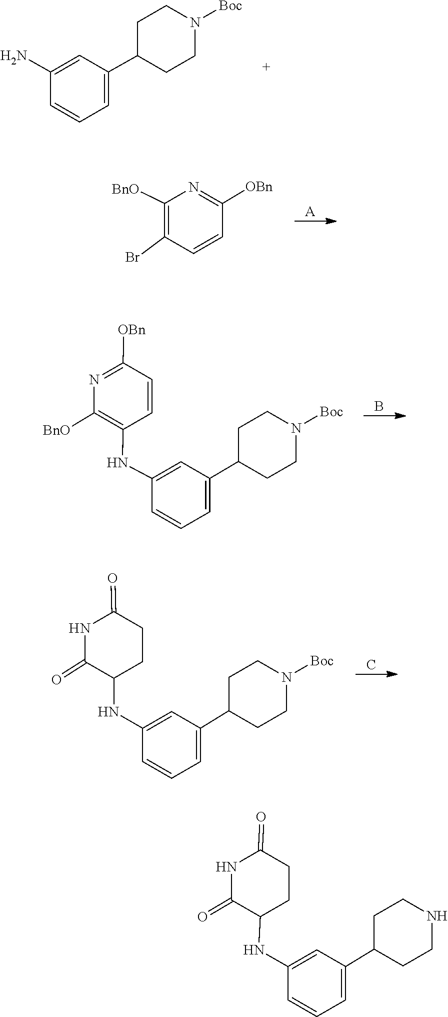

### Step A

A solution of tert-butyl 4-(3-aminophenyl)piperidine-1-carboxylate (2.0 g, 7.23 mmol, 1.0 equiv), 2,6-bis(benzyloxy)-3-bromopyridine (2.68 g, 7.24 mmol, 1.0 equiv), t-BuONa (1.04 g, 10.9 mmol, 1.5 equiv), Pd₂(dba)₃·CHCl₃ (0.75 g, 0.72 mmol, 0.1 equiv) and XantPhos (0.42 g, 0.72 mmol, 0.1 equiv) in toluene (30 mL) was stirred overnight at 100° C. The resulting mixture was concentrated under reduced pressure. The residue was purified by reverse phase flash chromatography with the following conditions: column, C18 silica gel; mobile phase, MeCN in water (10 mmol/L NH₄HCO₃), 0% to 100% gradient in 40 min; detector, UV 254 nm. This resulted in tert-butyl 4-(3-((2,6-bis(benzyloxy)pyridin-3-yl)amino)phenyl)piperidine-1-carboxylate (3.2 g, 78%) as a brown solid. LCMS (ESI, m/z): 566.25 [M+H]⁺.

### Step B

A solution of tert-butyl 4-(3-((2,6-bis(benzyloxy)pyridin-3-yl)amino)phenyl)piperidine-1-carboxylate (3.2 g, 5.66 mmol, 1.0 equiv) and Pd/C (3.0 g) in EtOAc (50 mL) was stirred for 30 min under hydrogen atmosphere. The resulting mixture was filtered and the filter cake was washed with CH₂Cl₂ (3×20 mL). The filtrate was concentrated under reduced pressure to afford tert-butyl 4-(3-((2,6-dioxopiperidin-3-yl)amino)phenyl)piperidine-1-carboxylate (2.2 g, 96%) as a dark, green solid. LCMS (ESI, m/z): 386.25 [M−H]⁻.

### Step C

A solution of tert-butyl 4-(3-((2,6-dioxopiperidin-3-yl)amino)phenyl)piperidine-1-carboxylate (2.3 g, 5.94 mmol, 1.0 equiv) in HCl in 1,4-dioxane (20 mL, 4 M) was stirred for 30 min. The resulting mixture was concentrated under reduced pressure to afford 3-((3-(piperidin-4-yl)phenyl)amino)piperidine-2,6-dione (2.4 g) as a dark green crude powder. The product was used in the next step without further purification. LCMS (ESI, m/z): 288.15 [M+H]⁺.

### Intermediate A11: Synthesis of 3-((6-(piperazin-1-yl)pyridin-3-yl)amino)piperidine-2,6-dione hydrochloride

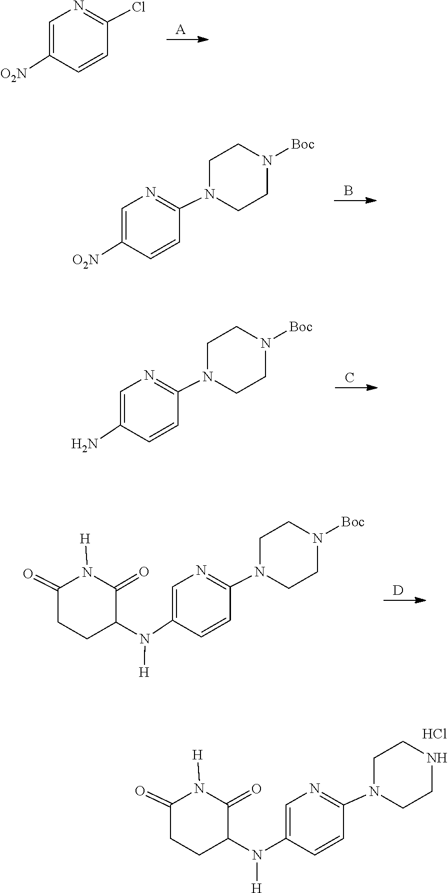

### Step A

A solution of 2-chloro-5-nitropyridine (5.0 g, 31.5 mmol, 1.0 equiv), tert-butyl piperazine-1-carboxylate (7.0 g, 37.9 mmol, 1.2 equiv) and K₂CO₃ (8.71 g, 63.1 mmol, 2.0 equiv) in DMF (10 mL) was stirred for 5 hours at 80° C. The mixture was allowed to cool down to room temperature. The precipitated solids were collected by filtration and washed with water (3×10 mL). The solid was dried in an oven to afford tert-butyl 4-(5-nitropyridin-2-yl)piperazine-1-carboxylate (9.6 g, 98%) as a yellow solid. LCMS (ESI, m/z): 309.15 [M+H]⁺.

### Step B

A solution of tert-butyl 4-(5-nitropyridin-2-yl)piperazine-1-carboxylate (9.6 g, 31.1 mmol, 1.0 equiv), Fe (10.4 g, 187 mmol, 6.0 equiv) and NH₄Cl (3.33 g, 62.3 mmol, 2.0 equiv) in water (10 mL) and EtOH (10 mL) was stirred overnight at 80° C. The resulting mixture was filtered and the filter cake was washed with EtOH (10×10 mL). The residue was purified by silica gel column chromatography, eluting with PE/EtOAc (1:1) to afford tert-butyl 4-(5-aminopyridin-2-yl)piperazine-1-carboxylate (8 g, 92%) as a red oil. LCMS (ESI, m/z): 279.20 [M+H]⁺.

### Step C

A solution of tert-butyl 4-(5-aminopyridin-2-yl)piperazine-1-carboxylate (2.0 g, 7.19 mmol, 1.0 equiv), 3-bromopiperidine-2,6-dione (1.66 g, 8.62 mmol, 1.2 equiv) and NaHCO₃ (1.81 g, 21.6 mmol, 3.0 equiv) in ACN (5 mL) was stirred overnight at 90° C. The mixture was cooled down to 0° C. The precipitated solids were collected by filtration and washed with water (2×8 mL). The solid was dried in an oven to afford tert-butyl 4-(5-((2,6-dioxopiperidin-3-yl)amino)pyridin-2-yl)piperazine-1-carboxylate (1.5 g, 54%) as a green solid. LCMS (ESI, m/z): 390.20 [M+H]⁺.

### Step D

A solution of tert-butyl 4-(5-((2,6-dioxopiperidin-3-yl)amino)pyridin-2-yl)piperazine-1-carboxylate (1.5 g, 3.85 mmol, 1.0 equiv) in HCl in 1,4-dioxane (6 mL, 4 M) was stirred for 30 min. The resulting mixture was concentrated under vacuum to afford 3-((6-(piperazin-1-yl)pyridin-3-yl)amino)piperidine-2,6-dione hydrochloride (1.0 g, 90%) as a purple crude solid. The product was used in the next step without further purification. LCMS (ESI, m/z): 290.15 [M+H]⁺.

Intermediates A11-a and A11-b were synthesized according to the procedure described for the synthesis of 3-((6-(piperazin-1-yl)pyridin-3-yl)amino)piperidine-2,6-dione hydrochloride (Intermediate A11) using appropriate building blocks and modified reaction conditions (such as reagents, reagent ratio, temperature, and reaction time) and purification conditions as needed.

| Example No. | Structure | Analytical Data |
|---|---|---|
| A11-a | 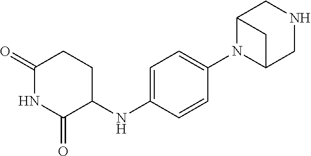 [SMILES: O=C1CCC(Nc2ccc(N3C4CNCC3C4)cc2)C(=O)N1] | LCMS (ESI, m/z): 301.20[M + H]⁺ |
| | 3-((4-(3,6-diazabicyclo[3.1.1]heptan-6- | |
| | yl)phenyl)amino)piperidine-2,6-dione | |
| A11-b | 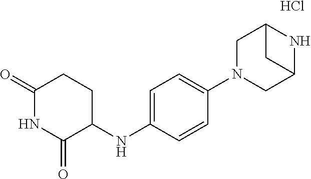 [SMILES: Cl ; O=C1CCC(Nc2ccc(N3CC4CC(C3)N4)cc2)C(=O)N1] | LCMS (ESI, m/z): 301.20[M + H]⁺ |
| | 3-((4-(3,6-diazabicyclo[3.1.1]heptan-3- | |
| | yl)phenyl)amino)piperidine-2,6-dione | |
| | hydrochloride | |
| A11-c | 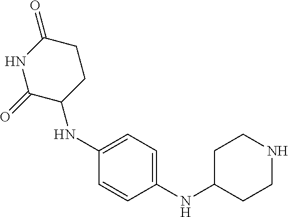 [SMILES: O=C1CCC(Nc2ccc(NC3CCNCC3)cc2)C(=O)N1] | LCMS (ESI, m/z): 303.30 [M + H]⁺ |
| | 3-((4-(piperidin-4- | |
| | ylamino)phenyl)amino)piperidine-2,6- | |
| | dione | |
| A11-d | 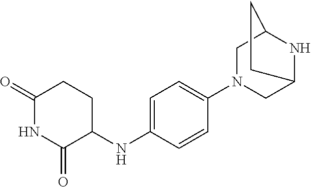 [SMILES: O=C1CCC(Nc2ccc(N3CC4CCC(C3)N4)cc2)C(=O)N1] | LCMS (ESI, m/z): 315.20 [M + H]⁺ |
| | 3-((4-(3,8-diazabicyclo[3.2.1]octan-3- | |
| | yl)phenyl)amino)piperidine-2,6-dione | |

### Intermediate A12: Synthesis of 2-(2,6-dioxopiperidin-3-yl)-5-(piperidin-4-ylethynyl)isoindoline-1,3-dione hydrochloride

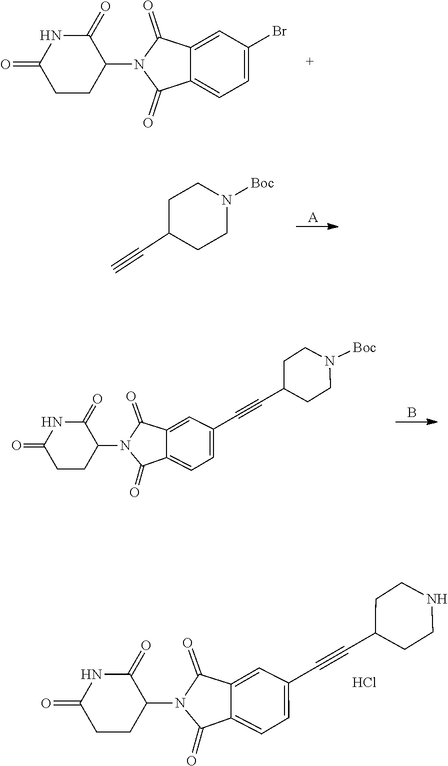

### Step A

A mixture of 5-bromo-2-(2,6-dioxopiperidin-3-yl)isoindoline-1,3-dione (1.58 g, 4.70 mmol, 1.0 equiv), tert-butyl 4-ethynylpiperidine-1-carboxylate (1.00 g, 4.79 mmol, 1.0 equiv), Pd(PPh₃)₂Cl₂ (329 mg, 0.47 mmol, 0.1 equiv) and TEA (1.42 g, 14.1 mmol, 3.0 equiv) in DMSO (7 mL) was stirred for 5 hours at 80° C. The mixture was cooled to room temperature and filtered. The filter cake was washed with DMSO (2 mL) and the filtrate was purified by C18 reverse phase chromatography eluting with water (10 mmol/L NH₄HCO₃)/ACN (55:45) to afford tert-butyl 4-((2-(2,6-dioxopiperidin-3-yl)-1,3-dioxoisoindolin-5-yl)ethynyl)piperidine-1-carboxylate (1.28 g, 59%) as a brown yellow solid. LCMS (ESI, m/z): 410.15 [M+H-t-Bu]⁺.

### Step B

A mixture of tert-butyl 4-((2-(2,6-dioxopiperidin-3-yl)-1,3-dioxoisoindolin-5-yl)ethynyl)piperidine-1-carboxylate (981 mg, 2.11 mmol) in HCl in 1,4-dioxane (30 mL, 4M) was stirred for 1.5 hours. The resulting mixture was concentrated under reduced pressure to afford 2-(2,6-dioxopiperidin-3-yl)-5-(piperidin-4-ylethynyl)isoindoline-1,3-dione hydrochloride (815 mg, 96%) as a white solid. The product was used in the next step without further purification. LCMS (ESI, m/z): 366.15 [M+H]⁺.

### Intermediate A13: Synthesis of 1-[1-methyl-6-(piperidin-4-yl)indazol-3-yl]-1,3-diazinane-2,4-dione

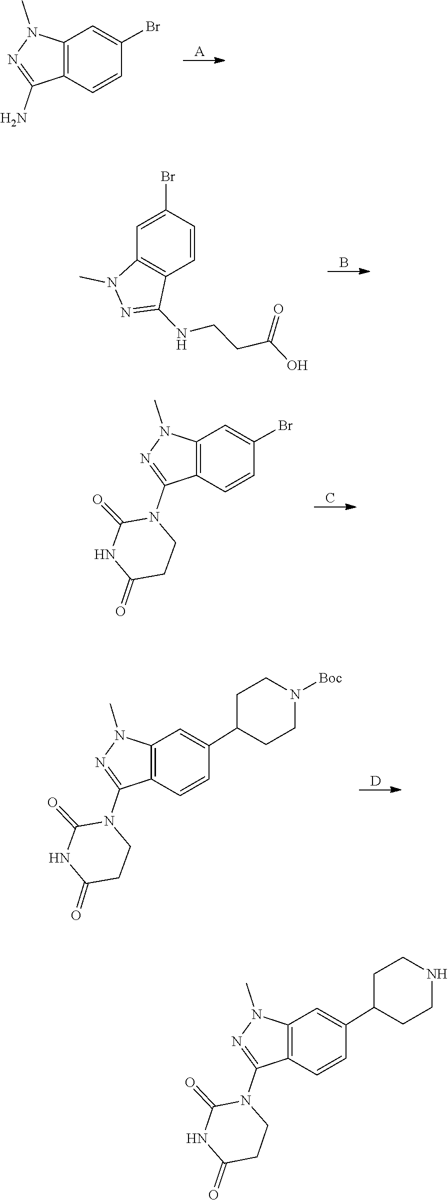

### Step A

A solution of 6-bromo-1-methylindazol-3-amine (10 g, 44.2 mmol, 1.0 equiv), acrylic acid (3.2 g, 44.4 mmol, 1.0 equiv) and HOAc (6.8 g, 113 mmol, 2.56 equiv) in water (100 mL) was stirred overnight at 100° C. Then NaOH (17.7 g, 443 mmol, 10 equiv) was added. The resulting mixture was extracted with EtOAc. The pH value of aqueous layer was adjusted to 3˜4 with aqueous HCl. The precipitated solids were collected by filtration and washed with water to afford 3-[(6-bromo-1-methylindazol-3-yl)amino]propanoic acid (8 g, 61%) as an off-white solid. LCMS (ESI, m/z): 298.15 [M+H]⁺.

### Step B

A solution of 3-[(6-bromo-1-methylindazol-3-yl)amino]propanoic acid (4.0 g, 13.4 mmol, 1.0 equiv) and urea (3.0 g, 50.0 mmol, 3.72 equiv) in HOAc (30 mL) was stirred overnight at 120° C. After concentration, the residue was purified by reverse phase flash chromatography with the following conditions: column, C18 silica gel; mobile phase, MeCN in water (0.1% HCl), 0% to 40% gradient in 15 min; detector, UV 254 nm. This afforded 1-(6-bromo-1-methylindazol-3-yl)-1,3-diazinane-2,4-dione (1.2 g, 14%) as a pink solid. LCMS (ESI, m/z): 323.15 [M+H]⁺.

### Step C

A solution of 1-(6-bromo-1-methylindazol-3-yl)-1,3-diazinane-2,4-dione (800 mg, 2.48 mmol, 1.0 equiv), tert-butyl 4-bromopiperidine-1-carboxylate (684 mg, 2.59 mmol, 1.05 equiv), NiBr₂·glyme (153 mg, 0.50 mmol, 0.20 equiv), dtbpy (133 mg, 0.5 mmol, 0.20 equiv), Mn (545 mg, 9.92 mmol, 4.01 equiv), NaI (372 mg, 2.48 mmol, 1.00 equiv) and pyridine (196 mg, 2.48 mmol, 1.00 equiv) in DMPU (15 mL) was stirred overnight at 70° C. under nitrogen atmosphere. The residue was purified by reversed-phase flash chromatography with the following conditions: column, C18 silica gel; mobile phase, MeCN in water (10 mmol/L NH₄HCO₃), 10% to 52% gradient in 15 min; detector, UV 254 nm. This afforded tert-butyl 4-[3-(2,4-dioxo-1,3-diazinan-1-yl)-1-methylindazol-6-yl]piperidine-1-carboxylate (230 mg, 22%) as a light yellow oil. LCMS (ESI, m/z): 428.50 [M+H]⁺.

### Step D

A solution of tert-butyl 4-[3-(2,4-dioxo-1,3-diazinan-1-yl)-1-methylindazol-6-yl]piperidine-1-carboxylate (230 mg, 0.54 mmol, 1.0 equiv) in TFA (5 mL) and DCM (5 mL) was stirred for 1 hour. The resulting mixture was concentrated under vacuum. The crude product was purified by prep-HPLC with the following conditions (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 26% B to 42% B in 9 min, 42% B; Wave Length: 254/220 nm; RT1 (min): 8.9). This afforded 1-[1-methyl-6-(piperidin-4-yl)indazol-3-yl]-1,3-diazinane-2,4-dione (110 mg, 62%) as a white solid. LCMS (ESI, m/z): 328.39 [M+H]⁺.

Intermediates A13-a was synthesized according to the procedure described for the synthesis of 1-[1-methyl-6-(piperidin-4-yl)indazol-3-yl]-1,3-diazinane-2,4-dione (Intermediate A13) using appropriate building blocks and modified reaction conditions (such as reagents, reagent ratio, temperature, and reaction time) and purification conditions as needed.

| Example No. | Structure | Analytical Data |
|---|---|---|
| A13-a | 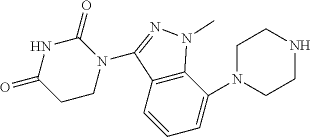 [SMILES: Cn1nc(N2CCC(=O)NC2=O)c2cccc(N3CCNCC3)c21] | LCMS (ESI, m/z): 329.16 [M + H]⁺ |
| | 1-(1-methyl-7-(piperazin-1-yl)-1H- | |
| | indazol-3-yl)dihydropyrimidine- | |
| | 2,4(1H,3H)-dione | |

### Intermediate A14: Synthesis of 3-((4-(((3aR,5r,6aS)-octahydrocyclopenta[c]pyrrol-5-yl)oxy)phenyl)amino)piperidine-2,6-dionehydrochloride

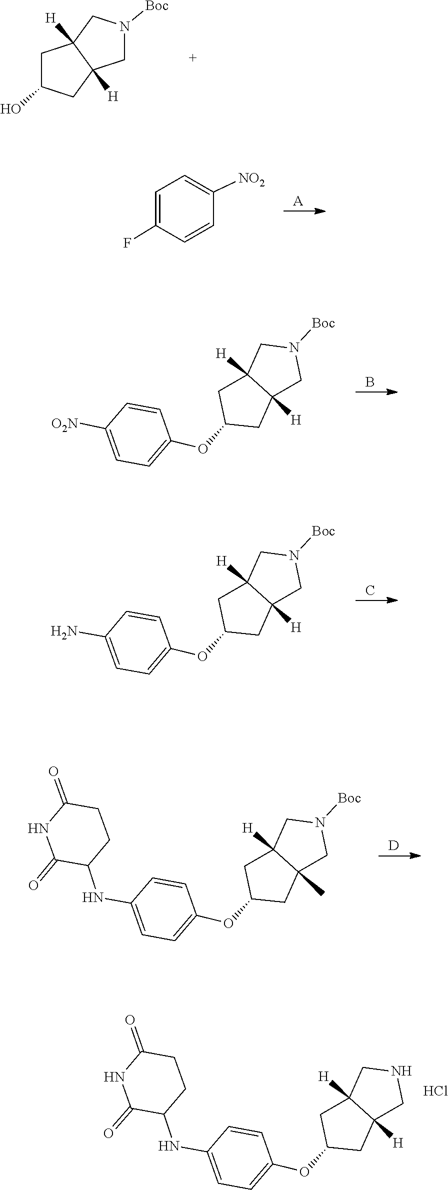

### Step A

A solution of tert-butyl (3aR,5r,6aS)-5-hydroxyhexahydrocyclopenta[c]pyrrole-2(1H)-carboxylate (1000 mg, 4.40 mmol, 1.0 equiv), 1-fluoro-4-nitrobenzene (931 mg, 6.6 mmol, 1.5 equiv) and t-BuOK (987 mg, 8.8 mmol, 2.0 equiv) in DMSO (6 mL) was stirred for 1 hour. The mixture was cooled to 0° C. The product was precipitated by the addition of water. The precipitated solids were collected by filtration and washed with water (3×10 mL) to afford tert-butyl (3aR,5r,6aS)-5-(4-nitrophenoxy)hexahydrocyclopenta[c]pyrrole-2(1H)-carboxylate (1.2 g, 78%) as a brown oil. LCMS (ESI, m/z): 349.15 [M+H]⁺.

### Step B

A solution of tert-butyl (3aR,5r,6aS)-5-(4-nitrophenoxy)hexahydrocyclopenta[c]pyrrole-2(1H)-carboxylate (1100 mg, 3.16 mmol, 1.0 equiv), Fe (882 mg, 15.8 mmol, 5.0 equiv) and NH₄Cl (338 mg, 6.31 mmol, 2.0 equiv) in EtOH (4 mL) and water (4 mL) was stirred for 3 hours at 80° C. The resulting mixture was filtered and the filter cake was washed with EtOH (10×10 mL). The filtrate was concentrated under reduced pressure. The residue was purified by silica gel column chromatography, eluting with PE/EtOAc (1:1) to afford tert-butyl (3aR,5r,6aS)-5-(4-aminophenoxy)hexahydrocyclopenta [c]pyrrole-2(1H)-carboxylate (825 mg, 82%) as a blue solid. LCMS (ESI, m/z): 319.20 [M+H]⁺.

### Step C

A solution of tert-butyl (3aR,5r,6aS)-5-(4-aminophenoxy)hexahydrocyclopenta[c]pyrrole-2(1H)-carboxylate (810 mg, 2.54 mmol, 1.0 equiv), 3-bromopiperidine-2,6-dione (733 mg, 3.82 mmol, 1.5 equiv) and NaHCO₃ (641 mg, 7.63 mmol, 3.0 equiv) in ACN (5 mL) was stirred overnight at 90° C. The residue was purified by silica gel column chromatography, eluting with PE/EtOAc (1:1) to afford tert-butyl (3aR,5r,6aS)-5-(4-((2,6-dioxopiperidin-3-yl)amino)phenoxy)hexahydrocyclopenta[c]pyrrole-2(1H)-carboxylate (804 mg, 74%) as a blue solid. LCMS (ESI, m/z): 429.23 [M+H]⁺.

### Step D

A solution of tert-butyl (3aR,5r,6aS)-5-(4-((2,6-dioxopiperidin-3-yl)amino)phenoxy)hexahydrocyclopenta[c]pyrrole-2(1H)-carboxylate (816 mg, 3.85 mmol, 1.0 equiv) in HCl in 1,4-dioxane (6 mL, 4 M) was stirred for 30 min. The resulting mixture was concentrated under vacuum to afford 3-((4-(((3aR,5r,6aS)-octahydrocyclopenta[c]pyrrol-5-yl)oxy) phenyl)amino)piperidine-2,6-dionehydrochloride (804 mg, 90%) as a grey crude solid. The product was used in the next step without further purification. LCMS (ESI, m/z): 330.17 [M+H]⁺.

### Intermediate A15: Synthesis of 1-(4-(piperidin-4-yl)phenyl)dihydropyrimidine-2,4(1H,3H)-dione hydrochloride

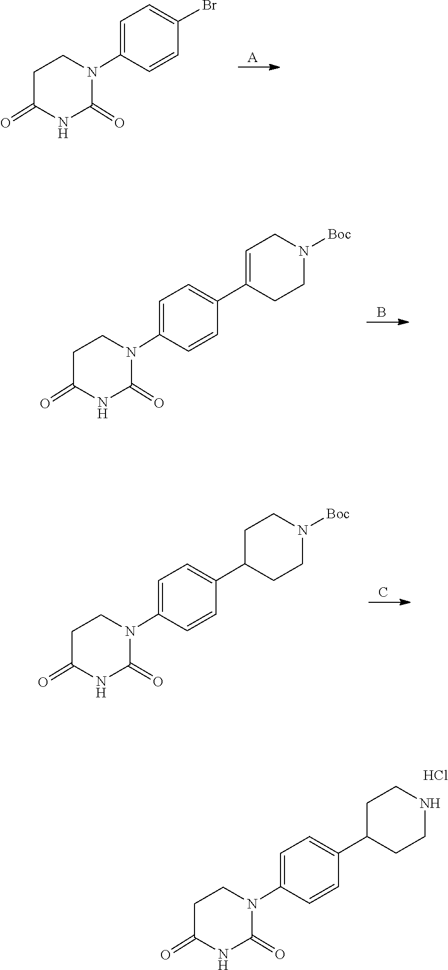

### Step A

A solution of 1-(4-bromophenyl)dihydropyrimidine-2,4(1H,3H)-dione (500 mg, 1.86 mmol, 1.0 equiv) and tert-butyl 4-(4,4,5,5-tetramethyl-1,3,2-dioxaborolan-2-yl)-3,6-dihydropyridine-1(2H)-carboxylate (689 mg, 2.23 mmol, 1.2 equiv), Pd(dppf)Cl₂CH₂Cl₂ (151 mg, 0.19 mmol, 0.1 equiv), K₃PO₄ (789 mg, 3.72 mmol, 2.0 equiv) in 1,4-dioxane (2 mL) and water (0.5 mL) was stirred for 1 hour at 80° C. After concentration, the residue was purified by silica gel column chromatography, eluting with PE/EtOAc (1:1) to afford tert-butyl 4-(4-(2,4-dioxotetrahydropyrimidin-1(2H)-yl)phenyl)-3,6-dihydropyridine-1(2H)-carboxylate (650 mg, 94%) as a white solid. LCMS (ESI, m/z): 370.10 [M−H]⁻.

### Step B

A solution of tert-butyl 4-(4-(2,4-dioxotetrahydropyrimidin-1(2H)-yl)phenyl)-3,6-dihydropyridine-1(2H)-carboxylate (650 mg, 1.75 mmol, 1.0 equiv) and Pd/C (186 mg) in EA (5 mL) was stirred for 1 hour under a hydrogen atmosphere. The resulting mixture was filtered and the filter cake was washed with EtOAc (3×9 mL). The filtrate was concentrated under reduced pressure to afford tert-butyl 4-(4-(2,4-dioxotetrahydropyrimidin-1(2H)-yl)phenyl)piperidine-1-carboxylate (620 mg, 95%) as a white solid. LCMS (ESI, m/z): 372.15 [M−H]⁻.

### Step C

A solution of tert-butyl 4-(4-(2,4-dioxotetrahydropyrimidin-1(2H)-yl)phenyl)piperidine-1-carboxylate (600 mg, 1.61 mmol, 1.0 equiv) in HCl in 1,4-dioxane (10 mL, 4 M) was stirred for 1 hour. The solution was concentrated to afford 1-(4-(piperidin-4-yl)phenyl)dihydropyrimidine-2,4(1H,3H)-dione hydrochloride (500 mg) as a white crude solid. The product was used in the next step without further purification. LCMS (ESI, m/z): 274.05 [M+H]⁺.

Intermediates A15-a through A15-d were synthesized according to the procedure described for the synthesis of 1-(4-(piperidin-4-yl)phenyl)dihydropyrimidine-2,4(1H,3H)-dione hydrochloride (Intermediate A15) using appropriate building blocks and modified reaction conditions (such as reagents, reagent ratio, temperature, and reaction time) and purification conditions as needed.

| Example No. | Structure | Analytical Data |
|---|---|---|
| A15-a | 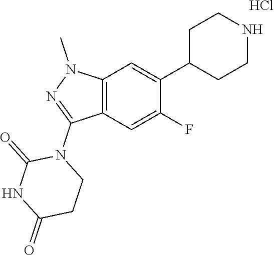 [SMILES: Cl ; Cn1nc(N2CCC(=O)NC2=O)c2cc(F)c(C3CCNCC3)cc21] | LCMS (ESI, m/z): 346.16 [M + H]⁺ |
| | 1-(5-fluoro-1-methyl-6-(piperidin-4- | |
| | yl)-1H-indazol-3- | |
| | yl)dihydropyrimidine-2,4(1H,3H)- | |
| | dion hydrochloride | |
| A15-b | 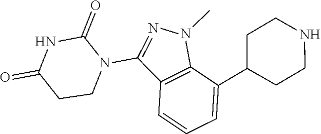 [SMILES: Cn1nc(N2CCC(=O)NC2=O)c2cccc(C3CCNCC3)c21] | LCMS (ESI, m/z): 328.15 [M + H]⁺ |
| | 1-(1-methyl-7-(piperidin-4-yl)-1H- | |
| | indazol-3-yl)dihydropyrimidine- | |
| | 2,4(1H,3H)-dione | |
| A15-c | 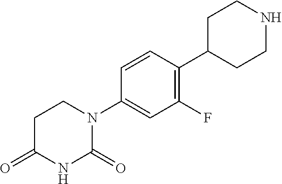 [SMILES: O=C1CCN(c2ccc(C3CCNCC3)c(F)c2)C(=O)N1] | LCMS (ESI, m/z): 292.30 [M + H]⁺ |
| | 1-(3-fluoro-4-(piperidin-4- | |
| | yl)phenyl)dihydropyrimidine- | |
| | 2,4(1H,3H)-dione | |
| A15-d | 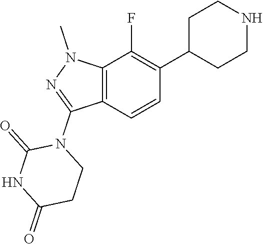 [SMILES: Cn1nc(N2CCC(=O)NC2=O)c2ccc(C3CCNCC3)c(F)c21] | LCMS (ESI, m/z): 346.15 [M + H]⁺ |
| | 1-(7-fluoro-1-methyl-6-(piperidin-4- | |
| | yl)-1H-indazol-3- | |
| | yl)dihydropyrimidine-2,4(1H,3H)- | |
| | dione | |

### Intermediate A16: Synthesis of 3-(3-methyl-2-oxo-5-(piperidin-4-yl)-2,3-dihydro-1H-benzo[d]imidazol-1-yl)piperidine-2,6-dione hydrochloric acid

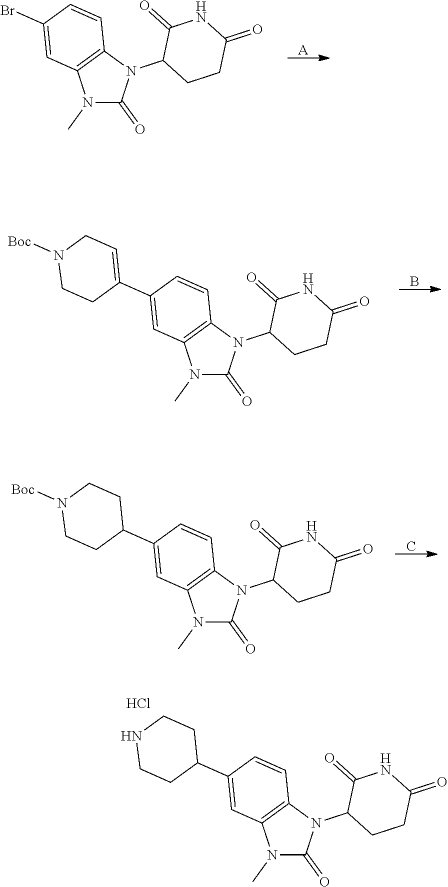

### Step A

A solution of 3-(5-bromo-3-methyl-2-oxo-2,3-dihydro-1H-benzo[d]imidazol-1-yl)piperidine-2,6-dione (1.0 g, 2.9 mmol, 1.0 equiv), tert-butyl 4-(4,4,5,5-tetramethyl-1,3,2-dioxaborolan-2-yl)-3,6-dihydro-2H-pyridine-1-carboxylate (1.82 g, 5.91 mmol, 2.0 equiv), K₃PO₄ (1.88 g, 8.87 mmol, 3.0 equiv) and Pd(dppf)Cl₂CH₂Cl₂ (241 mg, 0.296 mmol, 0.1 equiv) in dioxane (8 mL) was stirred for two days at 80° C. The resulting mixture was concentrated under reduced pressure. The residue was purified by silica gel column chromatography, eluting with CH₂Cl₂/MeOH (9:1) to afford 4-(1-(2,6-dioxopiperidin-3-yl)-3-methyl-2-oxo-2,3-dihydro-1H-benzo[d]imidazol-5-yl)-3,6-dihydropyridine-1(2H)-carboxylate (567 mg, 43%) as a white solid. LCMS (ESI, m/z): 441.15 [M+H]⁺.

### Step B

A of solution of tert-butyl 4-(1-(2,6-dioxopiperidin-3-yl)-3-methyl-2-oxo-2,3-dihydro-1H-benzo[d]imidazol-5-yl)-3,6-dihydropyridine-1(2H)-carboxylate (413 mg, 0.938 mmol, 1.0 equiv) and 10% Pd/C (2 g) in 7 mL EtOAc was stirred for 1 hour under an atmosphere of hydrogen. The solution was filtered through celite and the filtrate was concentrated under reduced pressure to give tert-butyl 4-(1-(2,6-dioxopiperidin-3-yl)-3-methyl-2-oxo-2,3-dihydro-1H-benzo[d]imidazol-5-yl)piperidine-1-carboxylate (243 mg, 59%) as a yellow solid. LCMS (ESI, m/z): 443.15[M+H]⁺.

### Step C

A solution of tert-butyl 4-(1-(2,6-dioxopiperidin-3-yl)-3-methyl-2-oxo-2,3-dihydro-1H-benzo[d]imidazol-5-yl)piperidine-1-carboxylate (210 mg, 0.475 mmol, 1.0 equiv) in hydrochloric acid in 1,4-dioxane (10 mL, 4M) was stirred for 2 hours at room temperature. The resulting mixture was concentrated under reduced pressure to give 3-(3-methyl-2-oxo-5-(piperidin-4-yl)-2,3-dihydro-1H-benzo[d]imidazol-1-yl)piperidine-2,6-dione hydrochloric acid (224 mg) as a brown crude oil. The crude product was used in the next step directly without further purification. LCMS (ESI, m/z):343.15[M+H]⁺.

### Intermediate A17: Synthesis of 3-(3-methyl-2-oxo-5-(piperazin-1-yl)-2,3-dihydro-1H-benzo[d]imidazol-1-yl)piperidine-2,6-dione hydrochloride

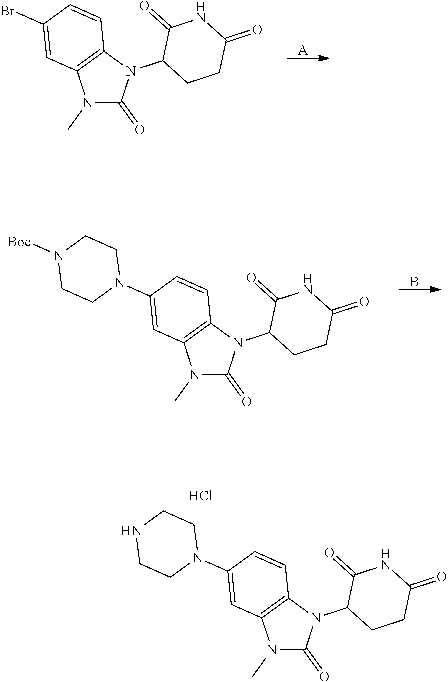

### Step A

A solution of 3-(5-bromo-3-methyl-2-oxo-2,3-dihydro-1H-benzo[d]imidazol-1-yl)piperidine-2,6-dione (500 mg, 1.48 mmol, 1 equiv) and tert-butyl piperazine-1-carboxylate (413 mg, 2.2 mmol, 1.5 equiv), RuPhos-PdCl-2nd G (115 mg, 0.15 mmol, 0.1 equiv) RuPhos (69.1 mg, 0.15 mmol, 0.1 equiv), and LiHMDS (2.9 mL, 2.9 mmol, 2 equiv, 1M in THF) in toluene (5 mL) was stirred overnight at 80° C. under nitrogen atmosphere. The resulting mixture was concentrated under vacuum. The residue was purified by reverse-phase flash chromatography with the following conditions: column, C18 silica gel; mobile phase, MeCN in water (0.1% TFA), 10% to 25% gradient in 20 min; detector, UV 254 nm. This afforded tert-butyl 4-(1-(2,6-dioxopiperidin-3-yl)-3-methyl-2-oxo-2,3-dihydro-1H-benzo[d]imidazol-5-yl)piperazine-1-carboxylate (220 mg, 33% yield) as a grey solid. LCMS (ESI, m/z): 444.20 [M+H]⁺.

### Step B

A solution of tert-butyl 4-(1-(2,6-dioxopiperidin-3-yl)-3-methyl-2-oxo-2,3-dihydro-1H-benzo[d]imidazol-5-yl)piperazine-1-carboxylate (220 mg, 0.50 mmol, 1 equiv) in HCl in 1,4-dioxane (5 mL, 4 M) was stirred for 1 hour. The resulting mixture was concentrated under vacuum to afford crude 3-(3-methyl-2-oxo-5-(piperazin-1-yl)-2,3-dihydro-1H-benzo[d]imidazol-1-yl)piperidine-2,6-dione hydrochloride (240 mg) as a yellow solid. The crude product was used in the next step directly without further purification. LCMS (ESI, m/z): 344.15 [M+H]⁺.

### Intermediate A18: Synthesis of 3-(isoindolin-5-ylamino)piperidine-2,6-dione

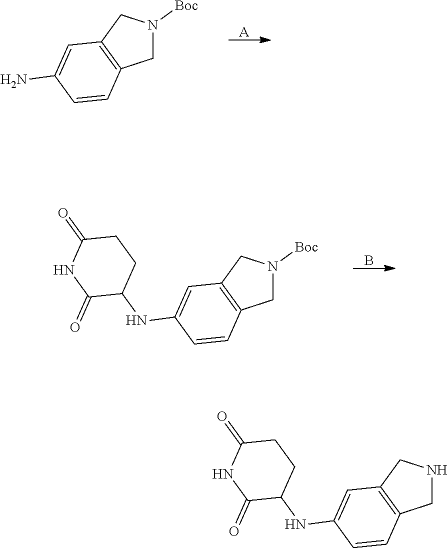

### Step A

A solution of tert-butyl 5-aminoisoindoline-2-carboxylate (3.0 g, 12.8 mmol, 1 equiv), 3-bromopiperidine-2,6-dione (4.92 g, 25.6 mmol, 2 equiv) and NaHCO₃ (5.38 g, 64.0 mmol, 5 equiv) in ACN (20 mL) was stirred for 2 days at 90° C. The solution was concentrated under vacuum and the residue applied to a silica gel column eluting with EtOAc/PE (50:50) to afford tert-butyl 5-((2,6-dioxopiperidin-3-yl)amino)isoindoline-2-carboxylate (5 g, 90%) as a dark blue solid. LCMS (ESI, m/z): 346.25 [M+H]⁺.

### Step B

A solution of tert-butyl 5-((2,6-dioxopiperidin-3-yl)amino)isoindoline-2-carboxylate (5.0 g, 14.5 mmol, 1 equiv) in HCl in 1,4-dioxane (40 mL, 4M) was stirred for 30 min. The resulting mixture was concentrated under vacuum to afford 3-(isoindolin-5-ylamino)piperidine-2,6-dione (3.4 g, 96%) as a brown solid. The product was used in the next step without further purification. LCMS (ESI, m/z): 246.20 [M+H]⁺.

Intermediate A18-b was synthesized according to the procedure described for the synthesis of 3-(isoindolin-5-ylamino)piperidine-2,6-dione hydrochloride (Intermediate A18) using appropriate building blocks and modified reaction conditions (such as reagents, reagent ratio, temperature, and reaction time) and purification conditions as needed.

| Example No. | Structure | Analytical Data |
|---|---|---|
| A18-b | 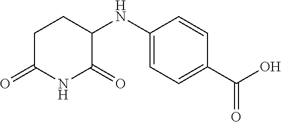 [SMILES: O=C1CCC(Nc2ccc(C(=O)O)cc2)C(=O)N1] | LCMS (ESI, m/z): 249.10 [M + H]⁺ |

### Intermediate A19: Synthesis of N-(2,6-dioxopiperidin-3-yl)-2-fluoro-4-(piperazin-1-yl)benzamide

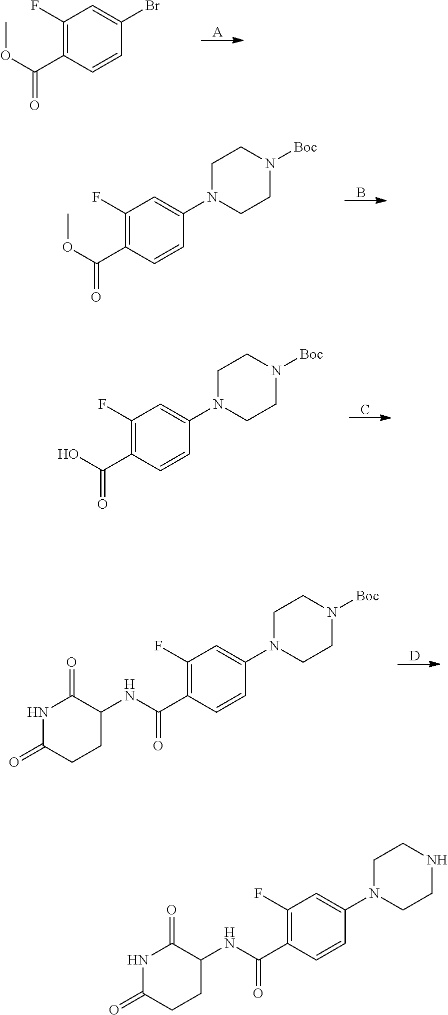

### Step A

A solution of methyl 4-bromo-2-fluorobenzoate (5.0 g, 21.5 mmol, 1 equiv) in dioxane (20 mL) was treated with tert-butyl piperazine-1-carboxylate (4.80 g, 25.7 mmol, 1.2 equiv), Pd(OAc)₂ (0.48 g, 2.15 mmol, 0.1 equiv) and BINAP (1.34 g, 2.15 mmol, 0.1 equiv) overnight at 100° C. under nitrogen atmosphere. The resulting mixture was concentrated under reduced pressure. The residue was purified by silica gel column chromatography, eluting with PE/EtOAc (72:28) to afford tert-butyl 4-(3-fluoro-4-(methoxycarbonyl)phenyl)piperazine-1-carboxylate (6 g, 83%) as a yellow solid. LCMS (ESI, m/z): 339.16 [M+H]⁺.

### Step B

A solution of tert-butyl 4-(3-fluoro-4-(methoxycarbonyl)phenyl)piperazine-1-carboxylate (2.0 g, 5.91 mmol, 1 equiv) in MeOH (5 mL) was treated with a solution of NaOH (4.73 g, 118 mmol, 20 equiv) in water (15 mL) for 3 hours. The mixture was acidified to pH 7 with concentrated HCl and the precipitated solids collected by filtration. This resulted in 4-(4-(tert-butoxycarbonyl)piperazin-1-yl)-2-fluorobenzoic acid (1.9 g, 99%) as a white solid. LCMS (ESI, m/z): 325.15 [M+H]⁺.

### Step C

A solution of 4-(4-(tert-butoxycarbonyl)piperazin-1-yl)-2-fluorobenzoic acid (1.9 g, 5.86 mmol, 1.0 equiv) in DMF (15 mL) was treated with 3-aminopiperidine-2,6-dione (0.90 g, 7.03 mmol, 1.2 equiv), DIEA (2.27 g, 17.6 mmol, 3.0 equiv), HOBT (1.19 g, 8.79 mmol, 1.5 equiv) and EDCI (1.68 g, 8.79 mmol, 1.5 equiv) overnight. The resulting mixture was concentrated under reduced pressure. The residue was purified by reverse-phase flash chromatography with the following conditions: column, C18 silica gel; mobile phase, MeCN in water (10 mmol/L NH₄HCO₃), 10% to 40% gradient in 15 min; detector, UV 254 nm to give tert-butyl 4-(4-((2,6-dioxopiperidin-3-yl)carbamoyl)-3-fluorophenyl)piperazine-1-carboxylate (1.29 g, 50%) as a grey solid. LCMS (ESI, m/z):435.20 [M+H]⁺.

### Step D

A solution of tert-butyl 4-(4-((2,6-dioxopiperidin-3-yl)carbamoyl)-3-fluorophenyl)piperazine-1-carboxylate (1.28 g, 2.94 mmol, 1 equiv) in HCl in 1,4-dioxane (10 mL, 4M) was stirred for 30 min. The resulting mixture was concentrated under reduced pressure to afford N-(2,6-dioxopiperidin-3-yl)-2-fluoro-4-(piperazin-1-yl)benzamide (1.35 g) as an off-white solid. The crude product was used in the next step directly without further purification. LCMS (ESI, m/z): 335.14 [M+H]⁺.

### Intermediate A20: Synthesis of N-methyl-N-(4-nitrophenyl)piperidin-4-amine

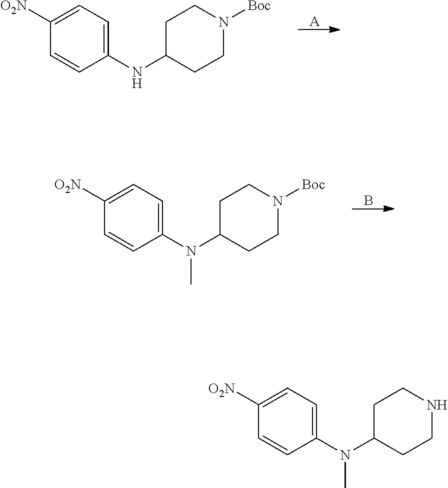

### Step A

A solution of tert-butyl 4-((4-nitrophenyl)amino)piperidine-1-carboxylate (4.0 g, 12.4 mmol, 1 equiv) in THF (25 mL) was treated with NaH (0.90 g, 37.3 mmol, 3 equiv) for 30 min at 0° C. followed by the addition of methyl iodide (3.53 g, 24.9 mmol, 2 equiv) dropwise at room temperature. The solution was stirred for 4 hours at room temperature. The reaction was quenched with water at 0° C. The aqueous layer was extracted with EtOAc (3×50 mL). The combined organic layers were concentrated under reduced pressure. This resulted tert-butyl 4-(methyl(4-nitrophenyl)amino)piperidine-1-carboxylate (4 g, 96%) as a yellow solid. LCMS (ESI, m/z): 336.15 [M+H]⁺.

### Step B

A solution of tert-butyl 4-(methyl(4-nitrophenyl)amino)piperidine-1-carboxylate (4.8 g, 14.3 mmol, 1 equiv) in HCl in 1,4-dioxane (20 mL, 4 M) was stirred for 30 min. The resulting mixture was concentrated under reduced pressure. This resulted in N-methyl-N-(4-nitrophenyl)piperidin-4-amine (4 g) as a yellow solid. The crude product was used in the next step directly without further purification. LCMS (ESI, m/z): 236.20 [M+H]⁺.

### Intermediate A21: Synthesis of 3-((2-fluoro-4-(piperazin-1-yl)phenyl)amino)piperidine-2,6-dione hydrochloride

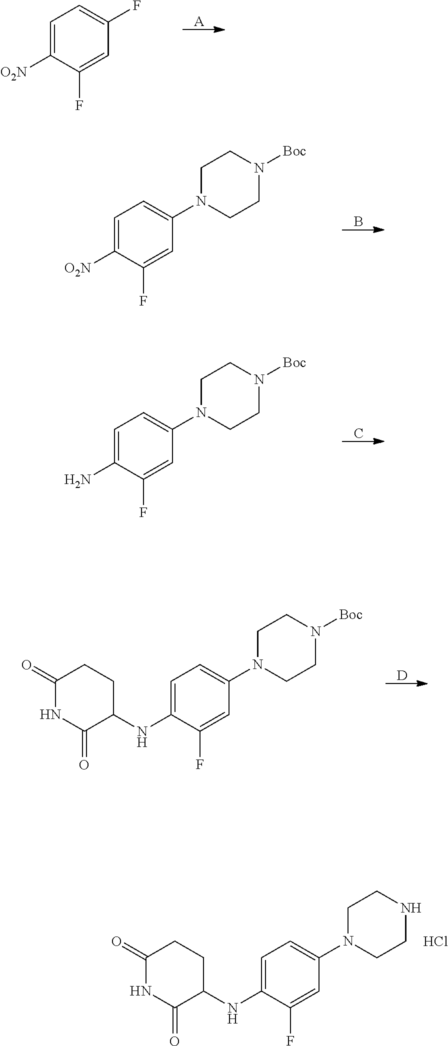

### Step A

A solution of 2,4-difluoro-1-nitrobenzene (5.0 g, 31.4 mmol, 1 equiv), tert-butyl piperazine-1-carboxylate (5.85 g, 31.4 mmol, 1 equiv) and TEA (9.54 g, 94.3 mmol, 3 equiv) in DMF (20 mL) was stirred for 5 hours at 80° C. The reaction was quenched with water and extracted with EtOAc (3×100 mL). The combined organic layers were washed with brine, dried over anhydrous sodium sulfate and concentrated under vacuum to give a mixture of regioisomers. The residue was purified by C18 reverse phase chromatography eluting with water/MeCN (40:60) to afford the desired isomer tert-butyl 4-(3-fluoro-4-nitrophenyl)piperazine-1-carboxylate (5 g, 48%) as a light yellow solid. LCMS (ESI, m/z): 326.15 [M+H]⁺.

### Step B

A solution of tert-butyl 4-(3-fluoro-4-nitrophenyl) piperazine-1-carboxylate (5 g, 15.1 mmol, 1 equiv), Fe (4.21 g, 75.3 mmol, 5 equiv) and NH₄Cl (1.61 g, 30.1 mmol, 2 equiv) in EtOH (10 mL) and water (10 mL) was stirred for 3 hours at 80° C. The resulting mixture was concentrated under vacuum was applied onto a silica gel column with MeOH/DCM (10:90) to afford tert-butyl 4-(4-amino-3-fluorophenyl)piperazine-1-carboxylate (3.3 g, 88%) as a black oil. LCMS (ESI, m/z): 296.15 [M+H]⁺.

### Step C

A solution of tert-butyl 4-(4-amino-3-fluorophenyl) piperazine-1-carboxylate (800 mg, 2.71 mmol, 1 equiv), 3-bromopiperidine-2,6-dione (1.56 g, 10.2 mmol, 2 equiv) and NaHCO₃ (1.13 g, 25.4 mmol, 5 equiv) in ACN (5 mL) was stirred for 2 days at 90° C. The resulting mixture was concentrated under vacuum, the residue was purified by C18 reverse phase chromatography eluting with water/ACN (33:67) to afford tert-butyl 4-(4-((2,6-dioxopiperidin-3-yl)amino)-3-fluorophenyl)piperazine-1-carboxylate (800 mg, 73%). LCMS (ESI, m/z): 407.20 [M+H]⁺.

### Step D

A solution of tert-butyl 4-(4-((2,6-dioxopiperidin-3-yl)amino)-3-fluorophenyl)piperazine-1-carboxylate (800 mg, 1.23 mmol, 1 equiv) in 1,4-dioxane (5 mL, 4 M) was stirred for 30 min. The resulting mixture was concentrated under vacuum to afford 3-((2-fluoro-4-(piperazin-1-yl)phenyl)amino)piperidine-2,6-dione hydrochloride (800 mg) as a black solid. The product was used in the next step without further purification. LCMS (ESI, m/z): 307.10 [M+H]⁺.

### Intermediate A22: Synthesis of 2-(4-(1-(4-nitrophenyl)piperidin-4-yl)piperazin-1-yl)ethan-1-ol

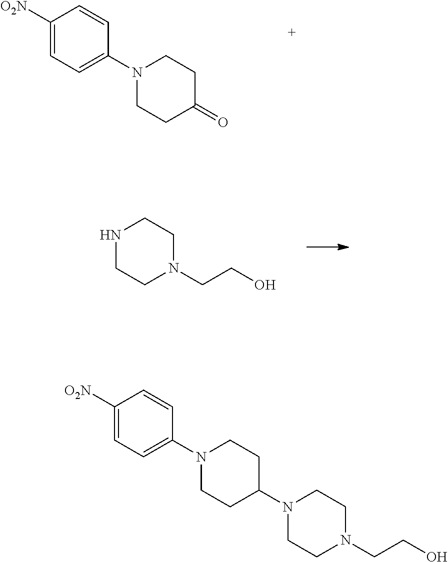 [SMILES: O=C1CCN(c2ccc([N+](=O)[O-])cc2)CC1 ; OCCN1CCNCC1 ; O=[N+]([O-])c1ccc(N2CCC(N3CCN(CCO)CC3)CC2)cc1]

A solution of 1-(4-nitrophenyl)piperidin-4-one (1.0 g, 4.54 mmol, 1 equiv), 2-(piperazin-1-yl)ethan-1-ol (0.59 g, 4.54 mmol, 1 equiv) and STAB (1.92 g, 9.08 mmol, 2 equiv) in DCE (30 mL) was stirred for 2 hours. After concentration, the residue was purified by reverse-phase flash chromatography with the following conditions: column, C18 silica gel; mobile phase, MeCN in water (10 mmol/L NH₄HCO₃), 0% to 50% gradient in 40 min; detector, UV 254 nm. This resulted in 2-(4-(1-(4-nitrophenyl)piperidin-4-yl)piperazin-1-yl)ethan-1-ol (455 mg, 30%) as a yellow oil. LCMS (ESI, m/z): 335.20 [M+H]⁺.

### Intermediate A23: Synthesis of 3-((3,5-difluoro-4-(piperazin-1-yl)phenyl)amino)piperidine-2,6-dione

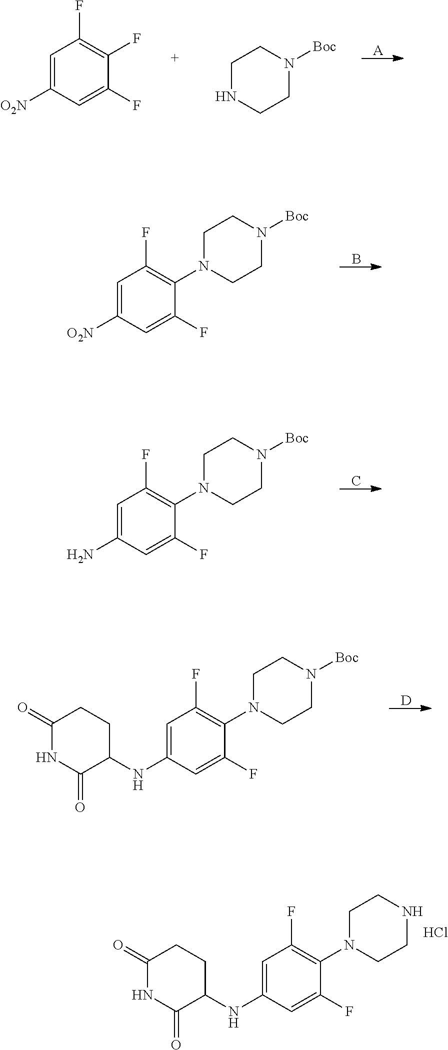

### Step A

A mixture of 1,2,3-trifluoro-5-nitrobenzene (5.0 g, 28.2 mmol, 1 equiv), tert-butyl piperazine-1-carboxylate (5.25 g, 28.2 mmol, 1.0 equiv) and TEA (8.56 g, 84.6 mmol, 3.0 equiv) in DMF (8 mL) was stirred for 1 hour at 80° C. After the addition of water, the precipitated solids were collected by filtration and washed with water to afford tert-butyl 4-(2,6-difluoro-4-nitrophenyl)piperazine-1-carboxylate (9.4 g, 97%) as a yellow solid. LCMS (ESI, m/z): 344.20 [M+H]⁺.

### Step B

A mixture of tert-butyl 4-(2,6-difluoro-4-nitrophenyl)piperazine-1-carboxylate (4.5 g, 13.1 mmol, 1 equiv), Fe (3.6 g, 64.5 mmol, 4.9 equiv) and NH₄Cl (2.1 g, 39.3 mmol, 3.0 equiv) in EtOH (20 mL) and water (5 mL) was stirred overnight at 80° C. The resulting mixture was filtered and the filter cake was washed with EtOH. The filtrate was concentrated under reduced pressure. The residue was purified by C18 reverse phase chromatography eluting with water (10 mmol/L NH₄HCO₃)/ACN (30:70). The resulting mixture in tert-butyl 4-(4-amino-2,6-difluorophenyl)piperazine-1-carboxylate (3 g, 73%) as a purple solid. LCMS (ESI, m/z): 314.15 [M+H]⁺.

### Step C

A mixture of tert-butyl 4-(4-amino-2,6-difluorophenyl)piperazine-1-carboxylate (1.5 g, 4.79 mmol, 1 equiv), 3-bromopiperidine-2,6-dione (7.3 g, 38.0 mmol, 7.94 equiv) and NaHCO₃ (3.6 g, 42.9 mmol, 8.95 equiv) in ACN (40 mL) was stirred for 2 days at 90° C. The resulting mixture was concentrated under reduced pressure. The residue was purified by silica gel column chromatography, eluting with DCM/MeOH (4:1) to afford tert-butyl 4-(4-((2,6-dioxopiperidin-3-yl)amino)-2,6-difluorophenyl)piperazine-1-carboxylate (3.45 g) as a white solid. LCMS (ESI, m/z): 425.10 [M+H]⁺.

### Step D

A mixture of tert-butyl 4-(4-((2,6-dioxopiperidin-3-yl)amino)-2,6-difluorophenyl)piperazine-1-carboxylate (1.0 g, 2.36 mmol, 1 equiv) in HCl in 1,4-dioxane (30 mL, 4 M) was stirred for 1 hour. The resulting mixture was concentrated under reduced pressure to afford 3-((3,5-difluoro-4-(piperazin-1-yl)phenyl)amino)piperidine-2,6-dione (990 mg, crude) as a white solid. LCMS (ESI, m/z): 325.20 [M+H]⁺.

### Intermediate A24: Synthesis of 2-(4-(1-(4-nitrophenyl)azetidin-3-yl)piperazin-1-yl)ethan-1-ol

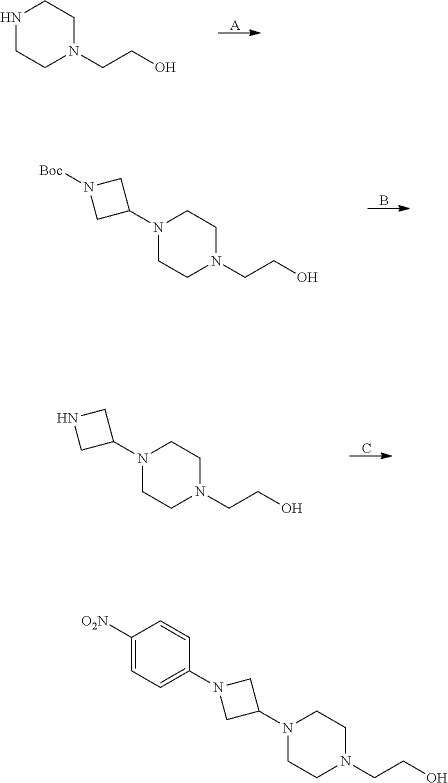

### Step A

A mixture of 2-(piperazin-1-yl)ethan-1-ol (5 g, 38.4 mmol, 1 equiv), tert-butyl 3-oxoazetidine-1-carboxylate (16.4 g, 96.0 mmol, 2.5 equiv), and STAB (16.3 g, 76.8 mmol, 2 equiv) in DCE (20 mL) was stirred for 2 hours. The residue was concentrated and purified by silica gel column chromatography, eluting with DCM/MeOH (92:8) to afford tert-butyl 3-(4-(2-hydroxyethyl)piperazin-1-yl)azetidine-1-carboxylate (10.3 g, 94%) as a yellow oil. LCMS (ESI, m/z): 286.15 [M+H]⁺.

### Step B

A solution of tert-butyl 3-(4-(2-hydroxyethyl)piperazin-1-yl)azetidine-1-carboxylate (5 g, 17.5 mmol, 1 equiv) in HCl in 1,4-dioxane (15 mL, 4 M) was stirred for 2 hours. The resulting mixture was concentrated under reduced pressure to give 2-(4-(azetidin-3-yl)piperazin-1-yl)ethan-1-ol (4.8 g) as a yellow oil. The crude product was used in the next step without further purification. LCMS (ESI, m/z): 186.15 [M+H]⁺.

### Step C

A solution of 2-(4-(azetidin-3-yl)piperazin-1-yl)ethan-1-ol (3.8 g, 20.5 mmol, 1 equiv), K₂CO₃ (8.50 g, 61.5 mmol, 3 equiv) and 1-fluoro-4-nitrobenzene (5.79 g, 41.0 mmol, 2 equiv) in DMF (20 mL) was stirred overnight at 80° C. After cooling to room temperature the mixture was quenched with water (40 mL). The precipitated solids were collected by filtration and washed with water. The solids were triturated with diethyl ether (30 mL). After filtration, 2-(4-(1-(4-nitrophenyl)azetidin-3-yl)piperazin-1-yl)ethan-1-ol (2.4 g, 39%) was isolated as a yellow solid. LCMS (ESI, m/z): 307.10 [M+H]⁺.

### Intermediate A25: Synthesis of 3-[1-methyl-6-(piperazin-1-yl)indazol-3-yl]piperidine-2,6-dione

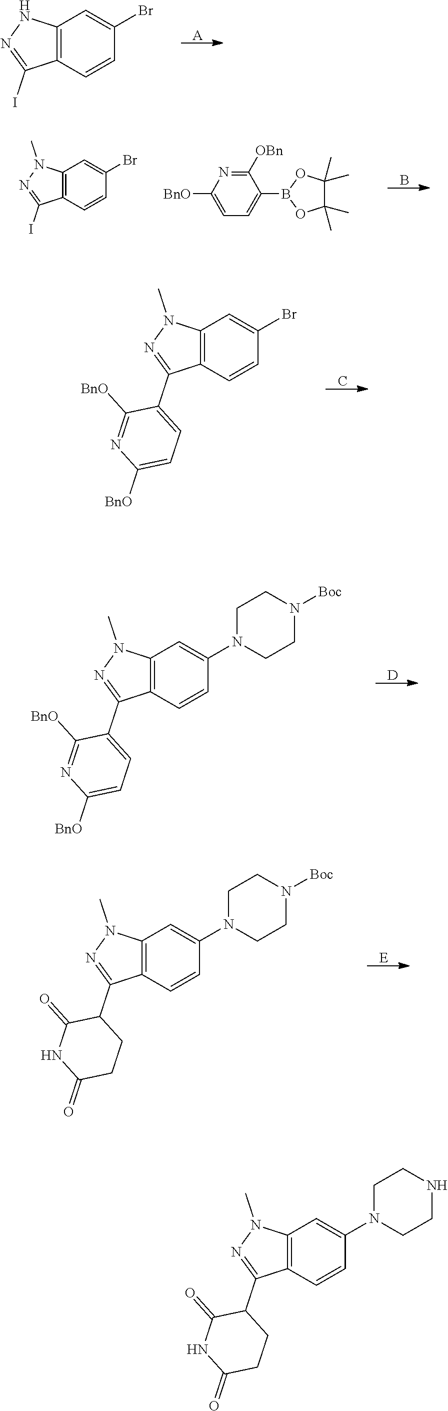

### Step A

To a mixture of 6-bromo-3-iodo-1H-indazole (500 mg, 1.54 mmol, 1.0 equiv) and NaH (111 mg, 4.64 mmol, 3.0 equiv) in DMF (2 mL) was added Mel (330 mg, 2.32 mmol, 1.5 equiv) at 0° C. under nitrogen atmosphere. The resulting mixture was stirred for 2 hours and then quenched with water at 0° C. and extracted with EtOAc (3×20 mL). The combined organic layers were washed with water (3×10 mL) and dried over anhydrous Na₂SO₄. After filtration, the filtrate was concentrated under reduced pressure. The residue was purified by silica gel column chromatography, eluting with PE/EtOAc (5:1) to afford 6-bromo-3-iodo-1-methylindazole (275 mg, 53%) as a yellow solid. LCMS (ESI, m/z): 336.8 [M+H]⁺.

### Step B

A solution of 6-bromo-3-iodo-1-methylindazole (500 mg, 1.48 mmol, 1.0 equiv) and 2,6-bis(benzyloxy)-3-(4,4,5,5-tetramethyl-1,3,2-dioxaborolan-2-yl)pyridine (929 mg, 2.23 mmol, 1.5 equiv) and K₂CO₃ (615 mg, 4.45 mmol, 3.0 equiv) in dioxane (2 mL) and water (1 mL) was stirred for 1 h at 80° C. under nitrogen atmosphere. The resulting mixture was filtered and the filter cake was washed with EtOAc (3×10 mL). The filtrate was concentrated under reduced pressure. The residue was purified by silica gel column chromatography, eluting with PE/EtOAc (1:1) to afford 3-[2,6-bis(benzyloxy)pyridin-3-yl]-6-bromo-1-methylindazole (490 mg, 66%) as a yellow oil. LCMS (ESI, m/z): 500.1[M+H]⁺.

### Step C

A solution of 3-[2,6-bis(benzyloxy)pyridin-3-yl]-6-bromo-1-methylindazole (1.7 g, 3.48 mmol, 1.0 equiv) and tert-butyl piperazine-1-carboxylate (1.0 g, 5.21 mmol, 1.5 equiv), Cs₂CO₃ (3.4 g, 10.4 mmol, 3.0 equiv), Ruphos (160 mg, 0.34 mmol, 0.1 equiv) and RuPhos Pd G3 (291 mg, 0.34 mmol, 0.1 equiv) in 1,4-dioxane (10 ml) was stirred for 1 hour at 90° C. under nitrogen atmosphere. The resulting mixture was filtered, the filter cake was washed with EtOAc (3×10 mL). The filtrate was concentrated under reduced pressure. The residue was purified by silica gel column chromatography, eluting with PE/EtOAc (1:1) to afford tert-butyl 4-{3-[2,6-bis(benzyloxy)pyridin-3-yl]-1-methylindazol-6-yl}piperazine-1-carboxylate (1.1 g, 52%) as a yellow oil. LCMS (ESI, m/z): 606.3 [M+H]⁺.

### Step D

To a mixture of tert-butyl 4-{3-[2,6-bis(benzyloxy)pyridin-3-yl]-1-methylindazol-6-yl}piperazine-1-carboxylate (3.5 g, 5.78 mmol, 1.0 equiv) in MeOH (250 mL) was added Pd/C (500 mg, 4.70 mmol, 0.8 equiv). The resulting mixture was stirred for 3 days under hydrogen atmosphere. The resulting mixture was filtered and the filter cake was washed with MeOH (3×100 mL). The filtrate was concentrated under reduced pressure. The residue was purified by reverse-phase flash chromatography with the following conditions: column, C18 silica gel; mobile phase, MeCN in water (0.1% FA), 10% to 90% gradient in 30 min; detector, UV 254 nm. This resulted in tert-butyl 4-[3-(2,6-dioxopiperidin-3-yl)-1-methylindazol-6-yl]piperazine-1-carboxylate (624 mg, 25%) as a purple solid. LCMS (ESI, m/z): 428.2 [M+H]⁺.

### Step E

A solution of tert-butyl 4-[3-(2,6-dioxopiperidin-3-yl)-1-methylindazol-6-yl]piperazine-1-carboxylate (400 mg, 0.94 mmol, 1.0 equiv) in dioxane (2 mL) was treated with HCl in 1,4-dioxane (2 mL, 4 M) and then stirred for 2 hours under nitrogen atmosphere. The resulting mixture was concentrated under vacuum. The residue was purified by trituration with diethyl ether (50 mL) to afford 3-[1-methyl-6-(piperazin-1-yl)indazol-3-yl]piperidine-2,6-dione (180 mg, 59%) as a yellow solid. LCMS (ESI, m/z): 328.15 [M+H]⁺.

Intermediate A25-b was synthesized according to the procedure described for steps A-D of the synthesis of 3-[1-methyl-6-(piperazin-1-yl)indazol-3-yl]piperidine-2,6-dione (Intermediate A25) using appropriate building blocks and modified reaction conditions (such as reagents, reagent ratio, temperature, and reaction time) and purification conditions as needed.

| Example No. | Structure | Analytical Data |
|---|---|---|
| A25-b | 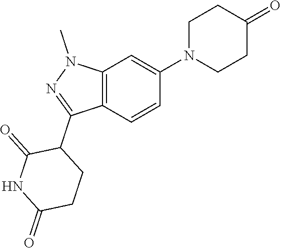 [SMILES: Cn1nc(C2CCC(=O)NC2=O)c2ccc(N3CCC(=O)CC3)cc21] | LCMS (ESI, m/z): 341.20 [M + H]⁺ |
| | 3-(1-methyl-6-(4-oxopiperidin-1-yl)- | |
| | 1H-indazol-3-yl)piperidine-2,6-dione | |

### Intermediate A26: Synthesis of (4-(4-((tert-butoxycarbonyl)amino)phenoxy)cyclohexyl)methyl 4-methylbenzenesulfonate

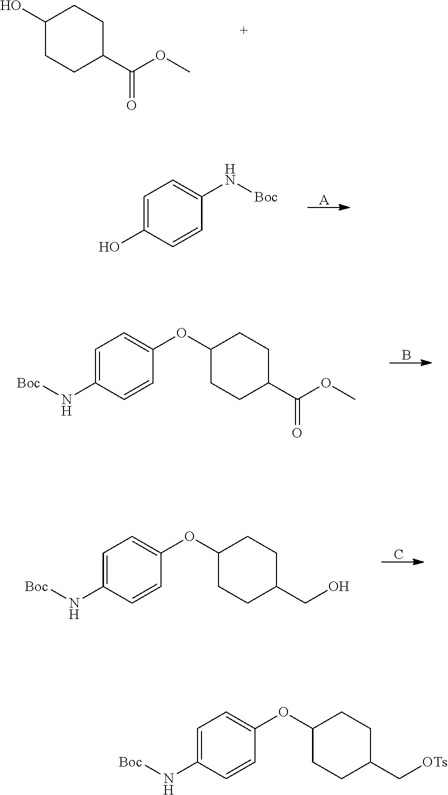

### Step A

To a solution of tert-butyl (4-hydroxyphenyl)carbamate (5.6 g, 26.8 mmol, 1.0 equiv), methyl 4-hydroxycyclohexane-1-carboxylate (6.35 g, 40.1 mmol, 1.5 equiv) and PPh₃ (10.5 g, 40.1 mmol, 1.5 equiv) in THF (50 mL) was added DEAD (6.99 g, 40.1 mmol, 1.5 equiv) portion-wise for 10 min at 0° C. The resulting mixture was stirred for 4 hours at 50° C. The solution were concentrated under vacuum and applied onto a silica gel column eluting with EtOAc/PE (30:70) to afford methyl 4-(4-((tert-butoxycarbonyl)amino)phenoxy)cyclohexane-1-carboxylate (4.4 g, 47%) as a brown oil. LCMS (ESI, m/z): 350.20 [M+H]⁺.

### Step B

A solution of methyl 4-(4-((tert-butoxycarbonyl)amino)phenoxy)cyclohexane-1-carboxylate (4.4 g, 12.6 mmol, 1 equiv) in THF (20 mL) was treated with LiAlH₄ (0.96 g, 25.2 mmol, 2 equiv) at 0° C. The resulting mixture was stirred for 2 hours at room temperature. The reaction was then quenched with water at 0° C. and extracted with EtOAc (3×100 mL). The combined organic extracts were concentrated under vacuum to afford tert-butyl (4-((4-(hydroxymethyl)cyclohexyl)oxy)phenyl)carbamate (3.7 g, 91%) as a crude brown oil. LCMS (ESI, m/z): 322.20 [M+H]⁺.

### Step C

A solution of tert-butyl (4-((4-(hydroxymethyl)cyclohexyl)oxy)phenyl)carbamate (500 mg, 1.56 mmol, 1 equiv) in DCM (10 mL) was treated with TEA (472 mg, 4.67 mmol, 3 equiv) for 5 min at room temperature followed by the addition of 4-methylbenzenesulfonyl chloride (356 mg, 1.87 mmol, 1.2 equiv) in portions at 0° C. The resulting mixture was stirred for 1 hour at room temperature. The solution was concentrated under vacuum and purified by silica gel column eluting with ethyl acetate/petroleum ether (20:80) to afford (4-(4-((tert-butoxycarbonyl)amino)phenoxy)cyclohexyl)methyl 4-methylbenzenesulfonate (550 mg, 74%) as a brown oil. LCMS (ESI, m/z): 476.20 [M+H]⁺.

### Intermediate A27: Synthesis of 3-(2-oxo-6-(piperazin-1-yl)benzo[cd]indol-1(2H)-yl)piperidine-2,6-dione hydrogen chloride

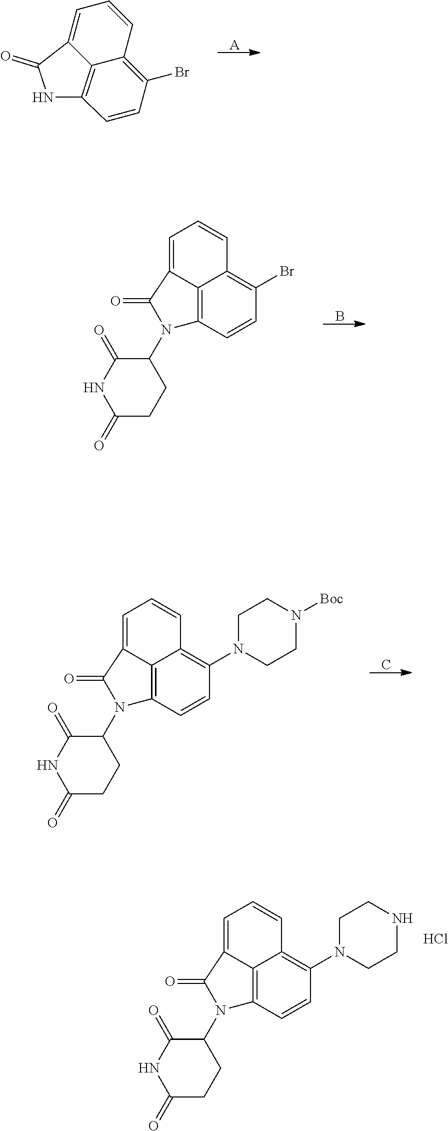

### Step A

To a solution of 6-bromobenzo[cd]indol-2(1H)-one (1 g, 4.03 mmol, 1 equiv) in THF (30 mL) was added NaH (0.48 g, 20.2 mmol, 5 equiv) in portions at 0° C. The resulting mixture was stirred for 1 hour at room temperature. To the above mixture was added 3-bromopiperidine-2,6-dione (1.93 g, 10.1 mmol, 2.5 equiv) at 0° C. The resulting mixture was stirred overnight at 60° C. The resulting mixture was concentrated under reduced pressure. The residue was purified by silica gel column chromatography, eluting with PE/EA (1:1) to afford 3-(6-bromo-2-oxobenzo[cd]indol-1(2H)-yl)piperidine-2,6-dione (169 mg, 12%) as a yellow solid. LCMS (ESI, m/z): 359.00, 361.00 [M+H]⁺

### Step B

A solution of 3-(6-bromo-2-oxobenzo[cd]indol-1(2H)-yl)piperidine-2,6-dione (149 mg, 0.42 mmol, 1 equiv) and tert-butyl piperazine-1-carboxylate (115.9 mg, 0.622 mmol, 1.5 equiv), Pd PEPPSI IPentCl (36 mg, 0.042 mmol, 0.1 equiv) and Cs₂CO₃ (203 mg, 0.622 mmol, 1.50 equiv) in dioxane (5 mL) was stirred for 3 hours at 90° C. The resulting mixture was concentrated under reduced pressure. The residue was purified by silica gel column chromatography, eluted with PE/EtOAc (1:1) to afford tert-butyl 4-(1-(2,6-dioxopiperidin-3-yl)-2-oxo-1,2-dihydrobenzo[cd]indol-6-yl)piperazine-1-carboxylate (162 mg, 84%) as a yellow solid. LCMS (ESI, m/z): 465.25 [M+H]⁺.

### Step C

A solution of tert-butyl 4-(1-(2,6-dioxopiperidin-3-yl)-2-oxo-1,2-dihydrobenzo[cd]indol-6-yl)piperazine-1-carboxylate (152 mg, 0.327 mmol, 1 equiv) in HCl in 1,4-dioxane (5 mL, 4 M) was stirred for 1 hour. The resulting mixture was concentrated under reduced pressure to afford crude 3-(2-oxo-6-(piperazin-1-yl)benzo[cd]indol-1(2H)-yl)piperidine-2,6-dione hydrogen chloride (153 mg) as a yellow solid. The crude was used in the next step without further purification. LCMS (ESI, m/z): 365.30 [M+H]⁺.

### Intermediate A28: Synthesis of 2-(6-(4-nitrophenyl)pyridin-3-yl)ethyl 4-methylbenzenesulfonate

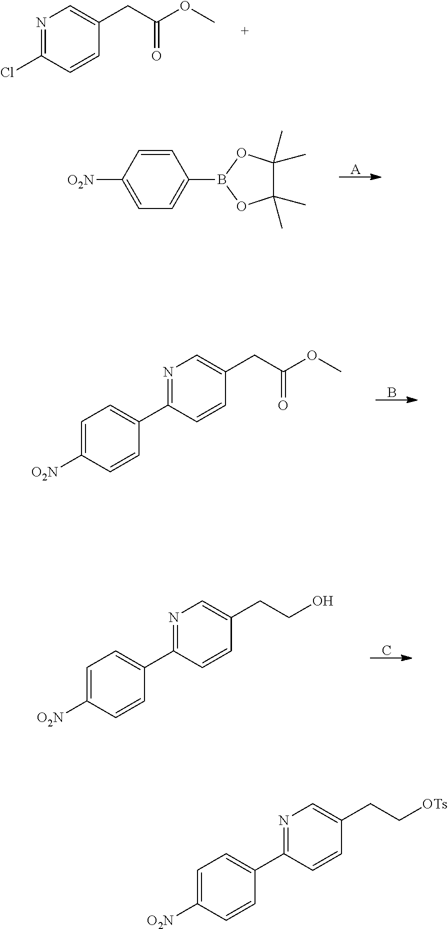

### Step A

A solution of methyl 2-(6-chloropyridin-3-yl)acetate (3.0 g, 16.2 mmol, 1 equiv), 4,4,5,5-tetramethyl-2-(4-nitrophenyl)-1,3,2-dioxaborolane (4.02 g, 16.2 mmol, 1 equiv), CsF (4.91 g, 32.3 mmol, 2 equiv) and Pd(PPh₃)₂Cl₂ (1.13 g, 1.62 mmol, 0.1 equiv) in water (6 mL) and EtOH (18 mL) was stirred for 1 hour at 100° C. under a nitrogen atmosphere. The resulting mixture was concentrated under reduced pressure. The residue was purified by silica gel column chromatography, eluting with DCM/MeOH (5:1) to afford methyl 2-(6-(4-nitrophenyl)pyridin-3-yl)acetate (3.1 g, 70%) as a yellow oil. LCMS (ESI, m/z): 273.25 [M+H]⁺.

### Step B

A solution of methyl 2-(6-(4-nitrophenyl)pyridin-3-yl)acetate (1.5 g, 5.51 mmol, 1 equiv), CaCl₂ (1.22 g, 11.0 mmol, 2 equiv) and NaBH₄ (0.417 g, 11.0 mmol, 2 equiv) in EtOH (10 mL) was stirred for 3 hours at 0° C. The resulting mixture was concentrated under reduced pressure. The residue was purified by silica gel column chromatography, eluting with DCM/MeOH (5:1) to afford 2-(6-(4-nitrophenyl)pyridin-3-yl)ethan-1-ol (900 mg, 67%) as a white solid. LCMS (ESI, m/z): 245.10 [M+H]⁺.

### Step C

A solution of 2-(6-(4-nitrophenyl)pyridin-3-yl)ethan-1-ol (500 mg, 2.05 mmol, 1 equiv), TEA (621 mg, 6.14 mmol, 3 equiv), TsCl (390 mg, 2.04 mmol, 1 equiv) and DMAP (25.0 mg, 0.205 mmol, 0.1 equiv) in DCM (5 mL) was stirred for 3 hours. The resulting mixture was concentrated under reduced pressure. The residue was purified by silica gel column chromatography, eluting with DCM/MeOH (5:1) to afford 2-(6-(4-nitrophenyl)pyridin-3-yl)ethyl 4-methylbenzenesulfonate (450 mg, 55%) as a yellow solid. LCMS (ESI, m/z): 399.20 [M+H]⁺.

Intermediate A28-a was synthesized according to the procedure described for the synthesis of 2-(6-(4-nitrophenyl)pyridin-3-yl)ethyl 4-methylbenzenesulfonate (Intermediate A28) using appropriate building blocks and modified reaction conditions (such as reagents, reagent ratio, temperature, and reaction time) and purification conditions as needed.

| Example No. | Structure | Analytical Data |
|---|---|---|
| A28-a | 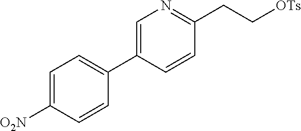 [SMILES: Cc1ccc(S(=O)(=O)OCCc2ccc(-c3ccc([N+](=O)[O-])cc3)cn2)cc1] | LCMS (ESI, m/z): 399.25 [M + H]⁺ |
| | 2-(5-(4-nitrophenyl)pyridin-2-yl)ethyl | |
| | 4-methylbenzenesulfonate | |

### Intermediate A29: Synthesis of 2-(4-(4-(2-fluoro-4-nitrophenyl)piperazin-1-yl)piperidin-1-yl)ethan-1-ol

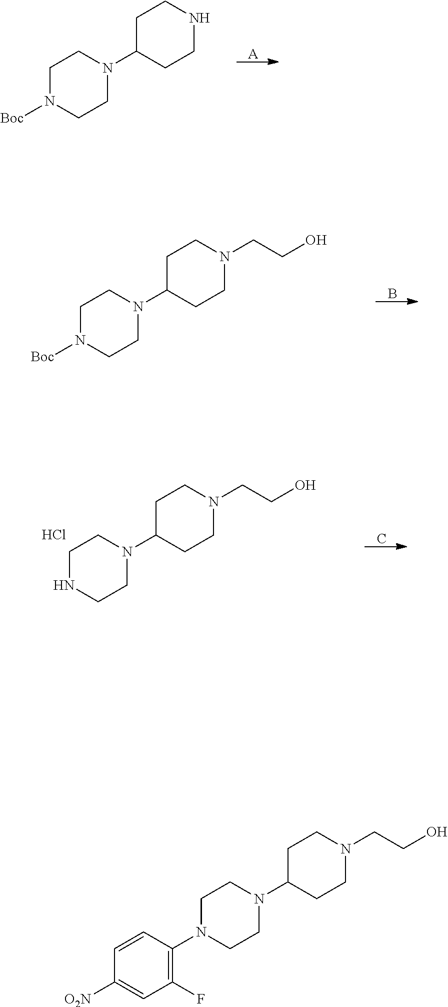

### Step A

A solution of tert-butyl 4-(piperidin-4-yl)piperazine-1-carboxylate (5 g, 18.6 mmol, 1 equiv), 2-bromoethan-1-ol (4.64 g, 37.1 mmol, 2 equiv) and K₂CO₃ (7.70 g, 55.7 mmol, 3 equiv) in DMF (25 mL) was stirred for 1 hour at 80° C. The reaction was quenched with water (50 mL) and extracted with EtOAc (4×50 mL). The combined organic layers were concentrated under reduced pressure to afford tert-butyl 4-(1-(2-hydroxyethyl)piperidin-4-yl)piperazine-1-carboxylate (8 g) as a yellow crude solid. LCMS (ESI, m/z): 314.25 [M+H]⁺.

### Step B

A solution of tert-butyl 4-(1-(2-hydroxyethyl)piperidin-4-yl)piperazine-1-carboxylate (8 g, 25.5 mmol, 1 equiv) in HCl in 1,4-dioxane (50 ml, 4 M) was stirred for 1 hour. The precipitated solids were collected by filtration and washed with PE (2×10 mL). The resulting solid was dried in the oven to offer 2-(4-(piperazin-1-yl)piperidin-1-yl)ethan-1-ol hydrochloride (7 g) as a white crude solid. The crude product was used in the next step without further purification. LCMS (ESI, m/z): 214.20 [M+H]⁺.

### Step C

A solution of 2-(4-(piperazin-1-yl)piperidin-1-yl)ethan-1-ol hydrochloride (6 g, 28 mmol, 1 equiv) and 1,2-difluoro-4-nitrobenzene (4.47 g, 28.2 mmol, 1 equiv), NaHCO₃ (11.8 g, 140.6 mmol, 5 equiv) in ACN (50 mL) was stirred for 1 hour at 80° C. The solution was concentrated and the residue was purified by silica gel column chromatography, eluting with DCM/MeOH (5:1) to afford 2-(4-(4-(2-fluoro-4-nitrophenyl)piperazin-1-yl)piperidin-1-yl)ethan-1-ol (3.64 g, 37%) as a yellow solid. LCMS (ESI, m/z): 353.20 [M+H]⁺.

### Intermediate A30: Synthesis of 3-((4-fluoro-3-(piperazin-1-yl)phenyl)amino)piperidine-2,6-dione hydrochloride

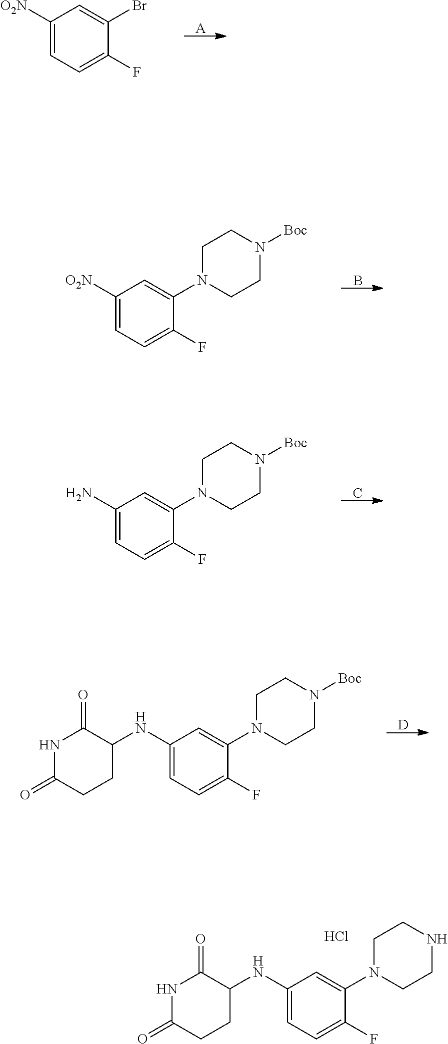

### Step A

A solution of 2-bromo-1-fluoro-4-nitrobenzene (5 g, 22.7 mmol, 1 equiv), tert-butyl piperazine-1-carboxylate (4.23 g, 22.7 mmol, 1 equiv), Pd(OAc)₂ (510 mg, 2.27 mmol, 0.1 equiv), XantPhos (1.32 g, 2.27 mmol, 0.1 equiv) and Cs₂CO₃ (14.8 g, 45.5 mmol, 2 equiv) in 1,4-dioxane (50 mL) was stirred overnight at 110° C. under nitrogen atmosphere. The resulting mixture was concentrated under reduced pressure. The residue was purified by silica gel column chromatography, eluting with PE/EA (1:1) to afford tert-butyl 4-(2-fluoro-5-nitrophenyl)piperazine-1-carboxylate (3.7 g, 50%) as a yellow solid. LCMS (ESI, m/z): 326.14 [M+H]⁺.

### Step B

A solution of tert-butyl 4-(2-fluoro-5-nitrophenyl)piperazine-1-carboxylate (2 g, 6.15 mmol, 1 equiv), Fe (1.72 g, 30.7 mmol, 5 equiv) and NH₄Cl (0.66 g, 12.3 mmol, 2 equiv) in EtOH (30 mL) and water (10 mL) was stirred for 1 hour at 80° C. The resulting mixture was concentrated under reduced pressure. The residue was purified by silica gel column chromatography, eluting with DCM/MeOH (8:1) to afford tert-butyl 4-(5-amino-2-fluorophenyl)piperazine-1-carboxylate (1.62 g, 89%) as a brown solid. LCMS (ESI, m/z): 296.17 [M+H]⁺.

### Step C

A solution of tert-butyl 4-(5-amino-2-fluorophenyl)piperazine-1-carboxylate (790 mg, 2.68 mmol, 1 equiv), 3-bromopiperidine-2,6-dione (1541 mg, 8.03 mmol, 3 equiv) and NaHCO₃ (449.4 mg, 5.35 mmol, 2 equiv) in ACN (15 mL) was stirred for 2 days at 90° C. The resulting mixture was concentrated under vacuum. The residue was purified by silica gel column chromatography, eluting with PE/EA (1:1) to afford tert-butyl 4-(5-((2,6-dioxopiperidin-3-yl)amino)-2-fluorophenyl)piperazine-1-carboxylate (1.35 g, quantitative) as a light blue solid. LCMS (ESI, m/z): 407.20 [M+H]⁺.

### Step D

A solution of tert-butyl 4-{5-[(2,6-dioxopiperidin-3-yl)amino]-2-fluorophenyl}piperazine-1-carboxylate (1.3 g, 3.2 mmol, 1 equiv) and HCl in 1,4-dioxane (10 mL, 4 M) was stirred for 14 hours. The precipitated solids were collected by filtration and washed with PE (3×5 mL) to give 3-((4-fluoro-3-(piperazin-1-yl)phenyl)amino)piperidine-2,6-dione hydrochloride (822 mg, 75%) as a light green solid. LCMS (ESI, m/z): 307.15 [M+H]⁺.

### Intermediate A31: Synthesis of (3-((2-fluoro-4-nitrophenyl)(methyl)amino)cyclobutyl)methyl 4-methylbenzenesulfonate

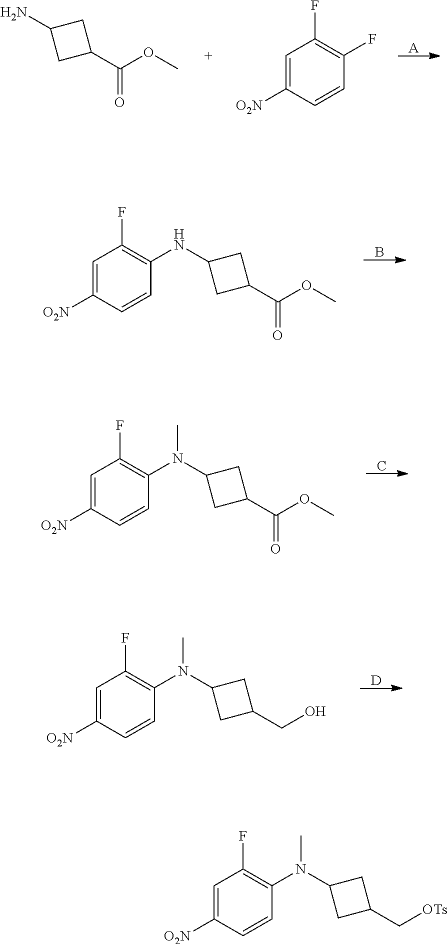

### Step A

A solution of methyl 3-aminocyclobutane-1-carboxylate (4.9 g, 37.9 mmol, 1 equiv), 1,2-difluoro-4-nitrobenzene (5.43 g, 34.1 mmol, 0.9 equiv) and DIEA (14.7 g, 114 mmol, 3 equiv) in NMP (30 mL) was stirred for 2 hours at 80° C. The reaction was quenched with water (50 mL). The resulting mixture was extracted with EtOAc (3×150 mL). The combined organic layers were washed with brine (3×100 mL), dried over anhydrous Na₂SO₄, filtered and concentrated under reduced pressure. The residue was purified by reverse phase flash chromatography with the following conditions: column, C18 silica gel; mobile phase, MeCN in water (10 mmol/L NH₄HCO₃), 10% to 50% gradient in 25 min; detector, UV 254 nm to give methyl 3-((2-fluoro-4-nitrophenyl)amino)cyclobutane-1-carboxylate (7.5 g, 74%) as a yellow solid. LCMS (ESI, m/z): 269.10 [M+H]⁺.

### Step B

To a stirred solution of methyl 3-((2-fluoro-4-nitrophenyl)amino)cyclobutane-1-carboxylate (4.5 g, 16.8 mmol, 1 equiv) and Cs₂CO₃ (10.9 g, 33.6 mmol, 2 equiv) in DMF (30 mL) was added methyl iodide (4.76 g, 33.6 mmol, 2 equiv) dropwise at room temperature. The resulting mixture was stirred for 2 hours. The reaction was quenched with water (50 mL). The resulting mixture was extracted with EtOAc (3×100 mL). The combined organic layers were washed with brine (3×60 mL), dried over anhydrous Na₂SO₄, filtered and concentrated under reduced pressure. The resulting residue was purified by silica gel column chromatography, eluting with PE/EA (41:9) to afford methyl 3-((2-fluoro-4-nitrophenyl)(methyl)amino)cyclobutane-1-carboxylate (4.1 g, 87%) as an orange oil. LCMS (ESI, m/z): 283.30 [M+H]⁺.

### Step C

To a solution of methyl 3-((2-fluoro-4-nitrophenyl)(methyl)amino)cyclobutane-1-carboxylate (1.0 g, 3.54 mmol, 1 equiv) in DCM (20 mL) under nitrogen atmosphere was added DIBAL-H (7.9 mL, 38.9 mmol, 11 equiv) dropwise at −65° C. The resulting mixture was stirred for 15 min at −65° C. and was then diluted with cold ethyl ether (20 mL). The mixture was warmed up to 0° C. The reaction was quenched with water (1 mL) and 15% NaOH (1 mL) at 0° C. The resulting mixture was stirred for 15 mins at room temperature. To the mixture was added anhydrous MgSO₄ portion-wise at room temperature. The resulting mixture was stirred for 15 mins then filtered and the filter cake washed with water (3×20 mL). The filtrate was concentrated under reduced pressure. The residue was purified by silica gel column chromatography, eluting with PE/EA (71:29) to afford (3-((2-fluoro-4-nitrophenyl)(methyl)amino)cyclobutyl)methanol (513 mg, 57%) as an orange oil. LCMS (ESI, m/z): 255.25 [M+H]⁺.

### Step D

A solution of (3-((2-fluoro-4-nitrophenyl)(methyl)amino)cyclobutyl)methanol (593 mg, 2.33 mmol, 1 equiv) in DCM (12 mL) was treated with TEA (472 mg, 4.66 mmol, 2 equiv) at room temperature followed by the addition of p-toluenesulfonyl chloride (667 mg, 3.5 mmol, 1.5 equiv) portion-wise at 0° C. The resulting mixture was stirred for 2 hours at room temperature then concentrated under reduced pressure. The residue was purified by silica gel column chromatography, eluting with PE/EA (19:6) to afford of (3-((2-fluoro-4-nitrophenyl)(methyl)amino)cyclobutyl)methyl 4-methylbenzenesulfonate (792 mg, 83%) as a yellow solid. LCMS (ESI, m/z): 409.75 [M+H]⁺.

### Intermediate A32: Synthesis of 3-((5-fluoro-6-(piperazin-1-yl)pyridin-3-yl)amino)piperidine-2,6-dione dione hydrochloride

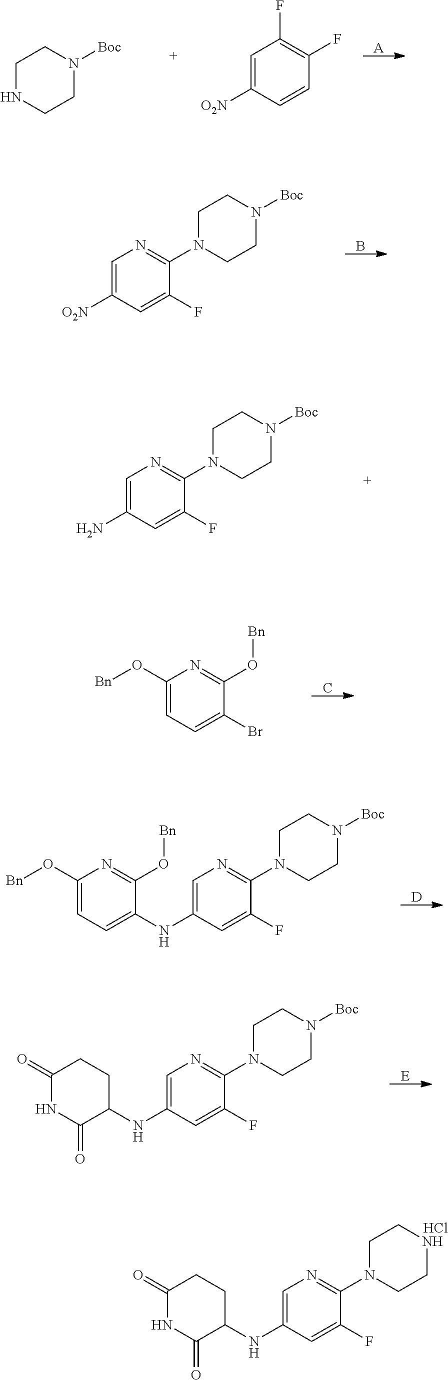

### Step A

A solution of tert-butyl piperazine-1-carboxylate (500 mg, 2.68 mmol, 1 equiv) and 2,3-difluoro-5-nitropyridine (430 mg, 2.68 mmol, 1 equiv) TEA (815 mg, 8.05 mmol, 3 equiv) in DMF (3 mL) was stirred for 2 hours at 80° C. The product was precipitated by the addition of water. The precipitated solids were collected by filtration and washed with water (2×10 mL). The resulting solids were dried in an oven to afford tert-butyl 4-(3-fluoro-5-nitropyridin-2-yl)piperazine-1-carboxylate (300 mg, 34%) as a yellow solid. LCMS (ESI, m/z): 327.15 [M+H]⁺.

### Step B

A solution of tert-butyl 4-(3-fluoro-5-nitropyridin-2-yl)piperazine-1-carboxylate (780 mg, 2.39 mmol, 1 equiv) and Pd/C (254 mg, 2.39 mmol, 1 equiv) in EtOAc (6 mL) was stirred for 2 hours under hydrogen atmosphere. The resulting mixture was filtered and the filter cake was washed with EtOAc (3×6 mL). The filtrate was concentrated under reduced pressure to afford tert-butyl 4-(5-amino-3-fluoropyridin-2-yl)piperazine-1-carboxylate (625 mg, 88%) as a yellow solid. LCMS (ESI, m/z): 297.15 [M+H]⁺.

### Step C

A solution of tert-butyl 4-(5-amino-3-fluoropyridin-2-yl)piperazine-1-carboxylate (600 mg, 2.03 mmol, 1 equiv) and 2,6-bis(benzyloxy)-3-bromopyridine (750 mg, 2.03 mmol, 1 equiv), t-BuONa (584 mg, 6.08 mmol, 3 equiv), Pd₂(dba)₃CHCl₃ (210 mg, 0.20 mmol, 0.1 equiv) and XantPhos (117 mg, 0.20 mmol, 0.1 equiv) in toluene (10 mL) was stirred overnight at 100° C. under nitrogen atmosphere. The mixture was concentrated under reduced pressure. The residue was purified by silica gel column chromatography, eluting with PE/EA (5:1) to afford tert-butyl 4-(5-((2,6-bis(benzyloxy)pyridin-3-yl)amino)-3-fluoropyridin-2-yl)piperazine-1-carboxylate (1 g, 84%) as a yellow solid. LCMS (ESI, m/z): 586.30 [M+H]⁺.

### Step D

A solution of tert-butyl 4-(5-((2,6-bis(benzyloxy)pyridin-3-yl)amino)-3-fluoropyridin-2-yl)piperazine-1-carboxylate (500 mg, 0.854 mmol, 1 equiv) in EtOAc (15 mL) was treated with Pd/C (91 mg, 0.85 mmol, 1 equiv) overnight at room temperature under hydrogen atmosphere. The resulting mixture was filtered, the filter cake was washed with EtOAc (2×10 mL). The filtrate was concentrated under reduced pressure to afford tert-butyl 4-(5-((2,6-dioxopiperidin-3-yl)amino)-3-fluoropyridin-2-yl)piperazine-1-carboxylate (330 mg, 95%) as a yellow solid. LCMS (ESI, m/z): 408.20 [M+H]⁺.

### Step E

A solution of tert-butyl 4-(5-((2,6-dioxopiperidin-3-yl)amino)-3-fluoropyridin-2-yl)piperazine-1-carboxylate (350 mg, 0.86 mmol, 1 equiv) in HCl in 1,4-dioxane (5 mL, 4 M) was stirred for 3 hours. The resulting mixture was concentrated under reduced pressure to afford 3-((5-fluoro-6-(piperazin-1-yl)pyridin-3-yl)amino)piperidine-2,6-dione hydrochloride (650 mg) as a dark green crude solid. The crude product was used in the next step without further purification. LCMS (ESI, m/z): 308.15 [M+H]⁺.

### Intermediate A33: Synthesis of 2-(4-((4-(2-fluoro-4-nitrophenyl)piperazin-1-yl)methyl)piperidin-1-yl)ethan-1-ol

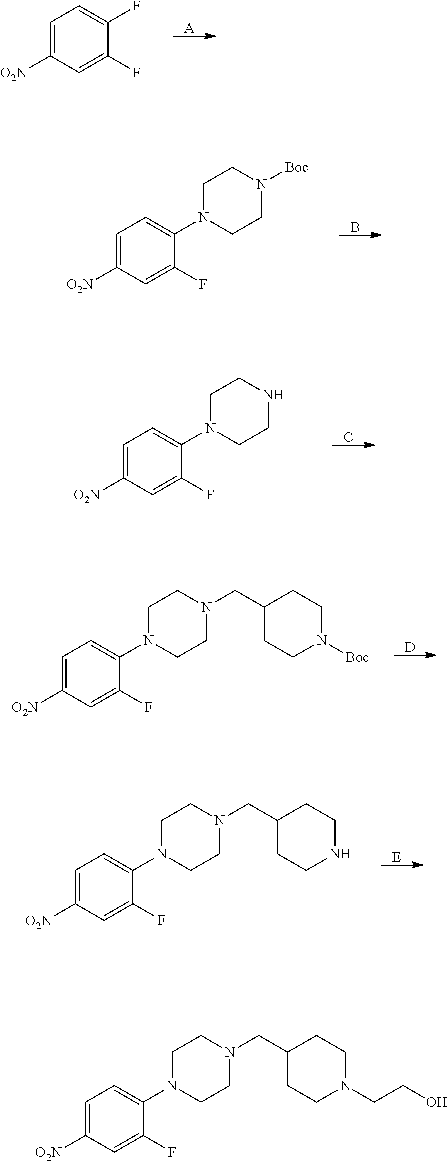

### Step A

A solution of 1,2-difluoro-4-nitrobenzene (5.0 g, 31.4 mmol, 1 equiv), tert-butyl piperazine-1-carboxylate (5.85 g, 31.4 mmol, 1 equiv) and NaHCO₃ (7.92 g, 94.3 mmol, 3 equiv) in ACN (120 mL) was stirred overnight at 90° C. The mixture was concentrated and the residue was purified by silica gel column chromatography, eluting with PE/EtOAc (1:1) to afford tert-butyl 4-(2-fluoro-4-nitrophenyl)piperazine-1-carboxylate (8.7 g, 85%) as a yellow solid. LCMS (ESI, m/z): 326.20 [M+H]⁺.

### Step B

A solution of tert-butyl 4-(2-fluoro-4-nitrophenyl)piperazine-1-carboxylate (8.7 g, 27 mmol, 1 equiv) in HCl in 1,4-dioxane (120 mL, 4 M) was stirred for 2 hours. The mixture was concentrated under vacuum to afford 1-(2-fluoro-4-nitrophenyl)piperazine (7.0 g) as a yellow solid. The crude product was used in the next step without further purification. LCMS (ESI, m/z): 226.20 [M+H]⁺.

### Step C

A solution of 1-(2-fluoro-4-nitrophenyl)piperazine (4.0 g, 18 mmol, 1 equiv), tert-butyl 4-formylpiperidine-1-carboxylate (4.55 g, 21.3 mmol, 1.2 equiv) and STAB (7.53 g, 35.5 mmol, 2 equiv) in DCE (120 mL) was stirred for 2 hours. The mixture was concentrated and the residue was purified by silica gel column chromatography, eluting with PE/EtOAc (1:1) to afford tert-butyl 4-((4-(2-fluoro-4-nitrophenyl)piperazin-1-yl)methyl)piperidine-1-carboxylate (7.9 g) as a crude yellow solid that was used without further purification. LCMS (ESI, m/z): 423.15 [M+H]⁺.

### Step D

A solution of tert-butyl 4-((4-(2-fluoro-4-nitrophenyl)piperazin-1-yl)methyl)piperidine-1-carboxylate (7.9 g, 19 mmol, 1 equiv) in HCl in 1,4-dioxane (150 mL, 4 M) was stirred for 2 hours. The mixture was concentrated under vacuum to afford 1-(2-fluoro-4-nitrophenyl)-4-(piperidin-4-ylmethyl)piperazine (7.0 g) as a yellow solid. The crude product was used in the next step without further purification. LCMS (ESI, m/z): 323.30 [M+H]⁺.

### Step E

A solution of 1-(2-fluoro-4-nitrophenyl)-4-(piperidin-4-ylmethyl)piperazine (2.5 g, 7.8 mmol, 1 equiv), 2-bromoethan-1-ol (0.97 g, 7.8 mmol, 1 equiv) and K₂CO₃ (4.29 g, 31.0 mmol, 4 equiv) in ACN (120 mL) was stirred for 6 hours at 80° C. The mixture was concentrated and the residue was purified by silica gel column chromatography, eluting with CH₂Cl₂/MeOH (5:1) to afford 2-(4-((4-(2-fluoro-4-nitrophenyl)piperazin-1-yl)methyl)piperidin-1-yl)ethan-1-ol (1.3 g, 46%) as a yellow oil. LCMS (ESI, m/z): 367.20 [M+H]⁺.

### Intermediate A34: Synthesis of methyl 2-fluoro-4-(4-oxopiperidin-1-yl)benzoate

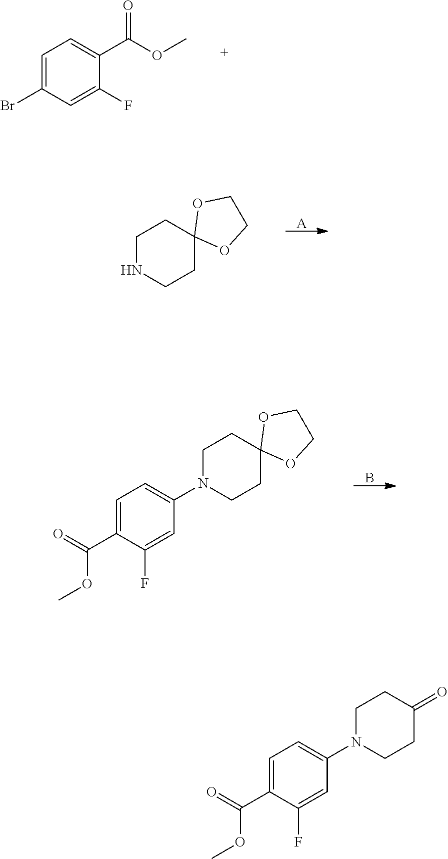

### Step A

A solution of methyl 4-bromo-2-fluorobenzoate (2.0 g, 8.6 mmol, 1 equiv), 1,4-dioxa-8-azaspiro[4.5]decane (1.11 g, 7.72 mmol, 0.9 equiv), Pd₂(dba)₃·CHCl₃ (0.89 g, 0.86 mmol, 0.1 equiv), XantPhos (0.50 g, 0.86 mmol, 0.1 equiv) and Cs₂CO₃ (5.59 g, 17.2 mmol, 2 equiv) in toluene (10 mL) was stirred overnight at 80° C. under nitrogen atmosphere. The mixture was concentrated and the residue was purified by silica gel column chromatography, eluting with PE/EtOAc (88:12) to afford methyl 2-fluoro-4-(1,4-dioxa-8-azaspiro[4.5]decan-8-yl)benzoate (988 mg, 39%) as a yellow solid. LCMS (ESI, m/z): 296.12 [M+H]⁺.

### Step B

A solution of methyl 2-fluoro-4-(1,4-dioxa-8-azaspiro[4.5]decan-8-yl)benzoate (978 mg, 3.31 mmol, 1 equiv) and HCl (5 mL, 2 M) in THF (5 mL) was stirred for 1 hour at 70° C. The mixture was neutralized to pH 7 with saturated NaHCO₃ (aq.). The aqueous layer was extracted with EtOAc (2×20 mL). The mixture was concentrated to dryness to afford methyl 2-fluoro-4-(4-oxopiperidin-1-yl)benzoate (822 mg, 99%) as a yellow oil. The crude product was used without further purification. LCMS (ESI, m/z): 252.10 [M+H]⁺.

Intermediate A34-a was synthesized according to the procedure described for the synthesis of methyl 2-fluoro-4-(4-oxopiperidin-1-yl)benzoate (Intermediate A34) using appropriate building blocks and modified reaction conditions (such as reagents, reagent ratio, temperature, and reaction time) and purification conditions as needed.

| Example | | |
|---|---|---|
| No. | Structure | Analytical Data |
| A34-a | 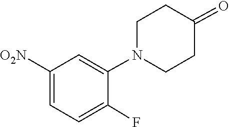 [SMILES: O=C1CCN(c2cc([N+](=O)[O-])ccc2F)CC1] | LCMS (ESI, m/z): 239.15 [M + H]⁺ |
| | 1-(2-fluoro-5-nitrophenyl)piperidin-4- | |
| | one | |

### Intermediates A35: Synthesis of 1-(3-fluoro-4-nitrophenyl)piperidin-4-one

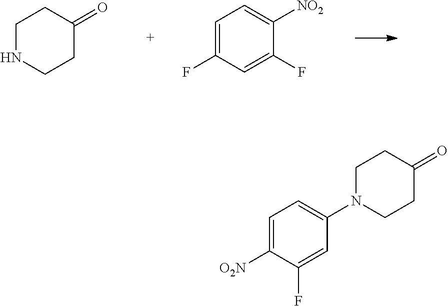 [SMILES: O=C1CCNCC1 ; O=[N+]([O-])c1ccc(F)cc1F ; O=C1CCN(c2ccc([N+](=O)[O-])c(F)c2)CC1]

A solution of 4-piperidinone (5.0 g, 50.4 mmol, 1 equiv), TEA (15.3 g, 151 mmol, 3 equiv) and 2,4-difluoro-1-nitrobenzene (12.0 g, 75.7 mmol, 1.5 equiv) in DMF (20 mL) was stirred for 4 hours at 80° C. After cooling to room temperature, the mixture was concentrated under reduced pressure. The residue was purified by silica gel column chromatography, eluting with PE/EA (85:15) to afford 1-(3-fluoro-4-nitrophenyl)piperidin-4-one (3.38 g, 28%) as a yellow solid. LCMS (ESI, m/z): 239.25 [M+H]⁺.

### Intermediate A36: Synthesis of 3-(5-fluoro-1-methyl-6-(piperidin-4-yl)-1H-indazol-3-yl)piperidine-2,6-dione hydrochloride

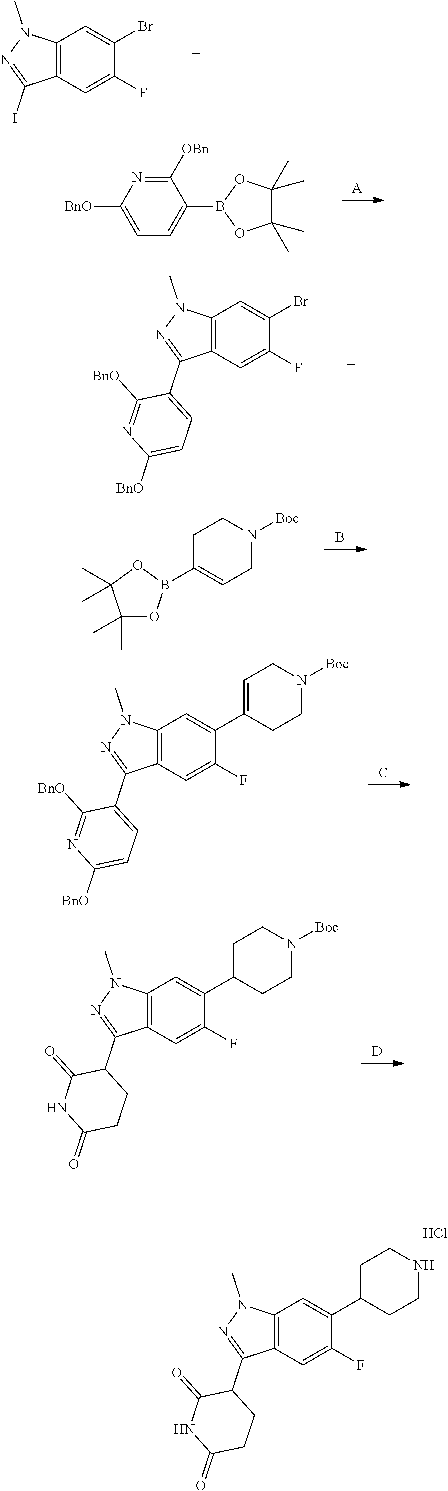

### Step A

A solution of 6-bromo-5-fluoro-3-iodo-1-methyl-1H-indazole (1.5 g, 4.23 mmol, 1 equiv), 2,6-bis(benzyloxy)-3-(4,4,5,5-tetramethyl-1,3,2-dioxaborolan-2-yl)pyridine (1.76 g, 4.23 mmol, 1 equiv), Pd(dppf)Cl₂ (0.31 g, 0.42 mmol, 0.1 equiv) and K₂CO₃ (1.17 g, 8.45 mmol, 2 equiv) in dioxane (8 mL) and water (4 mL) was stirred for 1 hour at 80° C. under nitrogen atmosphere. The mixture was concentrated and the residue was purified by silica gel column chromatography, eluting with PE/EA (8:1) to afford 3-(2,6-bis(benzyloxy)pyridin-3-yl)-6-bromo-5-fluoro-1-methyl-1H-indazole (1.0 g, 46%) as a yellow solid. LCMS (ESI, m/z): 518.35 [M+H]⁺.

### Step B

A solution of 3-(2,6-bis(benzyloxy)pyridin-3-yl)-6-bromo-5-fluoro-1-methyl-1H-indazole (1.0 g, 1.93 mmol, 1 equiv), tert-butyl 4-(4,4,5,5-tetramethyl-1,3,2-dioxaborolan-2-yl)-3,6-dihydropyridine-1(2H)-carboxylate (597 mg, 1.93 mmol, 1 equiv), Pd(dppf)Cl₂ (141 mg, 0.193 mmol, 0.1 equiv) and K₂CO₃ (533 mg, 3.86 mmol, 2 equiv) in dioxane (0.8 mL) and water (0.4 mL) was stirred for 1 hour at 80° C. under nitrogen atmosphere. The mixture was concentrated and the residue purified by silica gel column chromatography, eluting with PE/EtOAc (7:1) to afford tert-butyl 4-(3-(2,6-bis(benzyloxy)pyridin-3-yl)-5-fluoro-1-methyl-1H-indazol-6-yl)-3,6-dihydropyridine-1(2H)-carboxylate (1.0 g, 84%) as a yellow solid. LCMS (ESI, m/z): 621.15 [M+H]⁺.

### Step C

A solution of tert-butyl 4-(3-(2,6-bis(benzyloxy)pyridin-3-yl)-5-fluoro-1-methyl-1H-indazol-6-yl)-3,6-dihydropyridine-1(2H)-carboxylate (1.0 g, 1.6 mmol, 1 equiv) and 10% Pd/C (200 mg) in MeOH (60 mL) was stirred for 1 hour under hydrogen atmosphere. The mixture was filtered and the filter cake was washed with MeOH (4×25 mL). The filtrate was concentrated under reduced pressure to give tert-butyl 4-(3-(2,6-dioxopiperidin-3-yl)-5-fluoro-1-methyl-1H-indazol-6-yl)piperidine-1-carboxylate (653 mg, 91%) as a grey solid. LCMS (ESI, m/z): 445.25 [M+H]⁺.

### Step D

A solution of tert-butyl 4-(3-(2,6-dioxopiperidin-3-yl)-5-fluoro-1-methyl-1H-indazol-6-yl)piperidine-1-carboxylate (640 mg, 1.44 mmol, 1 equiv) and HCl in 1,4-dioxane (20 mL, 4M) was stirred for 1 hour. The mixture was concentrated to dryness to give 3-(5-fluoro-1-methyl-6-(piperidin-4-yl)-1H-indazol-3-yl)piperidine-2,6-dione hydrochloride (600 mg) as a grey solid. The crude product was used in the next step directly without further purification. LCMS (ESI, m/z): 345.25 [M+H]⁺.

### Intermediate A37: Synthesis of 1-(3-fluoro-4-(4-oxopiperidin-1-yl)phenyl)dihydropyrimidine-2,4(1H,3H)-dione

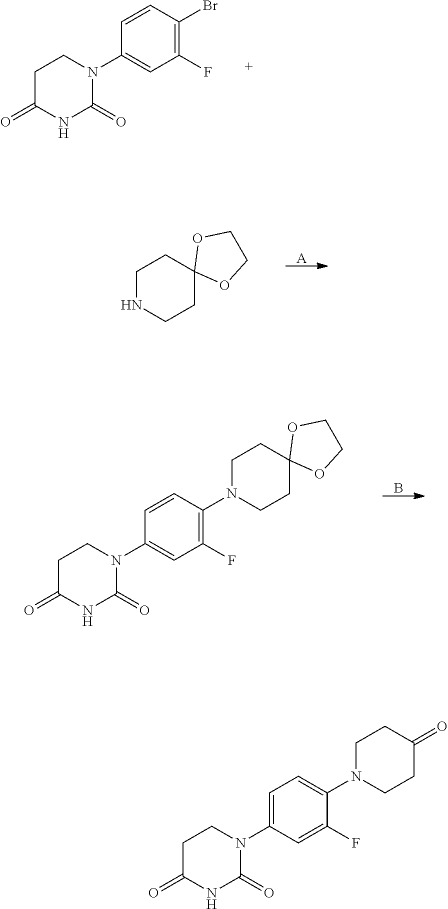

### Step A

A solution of 1-(4-bromo-3-fluorophenyl)dihydropyrimidine-2,4(1H,3H)-dione (500 mg, 1.74 mmol, 1 equiv), 1,4-dioxa-8-azaspiro[4.5]decane (374 mg, 2.61 mmol, 1.5 equiv), Cs₂CO₃ (1.14 g, 3.48 mmol, 2 equiv) and Pd PEPPSI IPentCl (147 mg, 0.174 mmol, 0.1 equiv) in dioxane (8 mL) was stirred for 1 hour at 85° C. under nitrogen atmosphere. The mixture was concentrated and the residue was purified by silica gel column chromatography, eluting with PE/EtOAc (1:3) to afford 1-(3-fluoro-4-(1,4-dioxa-8-azaspiro[4.5]decan-8-yl)phenyl)dihydropyrimidine-2,4(1H,3H)-dione (520 mg, 85%) as a white solid. LCMS (ESI, m/z): 350.35 [M+H]⁺.

### Step B

A solution of 1-(3-fluoro-4-(1,4-dioxa-8-azaspiro[4.5]decan-8-yl)phenyl)dihydropyrimidine-2,4(1H,3H)-dione (510 mg, 1.46 mmol, 1 equiv) and HCl (10 mL, 6 M) in TIFF (10 mL) was stirred overnight. The mixture was neutralized to pH 7 with saturated aqueous NaHCO₃. The mixture was extracted with DCM (3×50 mL). The combined organic layers were dried over anhydrous Na₂SO₄. After filtration, the filtrate was concentrated under reduced pressure to 1-(3-fluoro-4-(4-oxopiperidin-1l-yl)phenyl)dihydropyrimidine-2,4(1H,3H)-dione (460 mg) as a white solid. The crude product was used in the next step directly without further purification. LCMS (ESI, m/z): 306.15 [M+H]⁺.

Intermediates A37-a to A37-c were synthesized according to the procedure described for the synthesis of 1-(3-fluoro-4-(4-oxopiperidin-1l-yl)phenyl)dihydropyrimidine-2,4(1H,3H)-dione (Intermediate A37) using appropriate building blocks and modified reaction conditions (such as reagents, reagent ratio, temperature, and reaction time) and purification conditions as needed.

| Example No. | Structure | Analytical Data |
|---|---|---|
| A37-a | 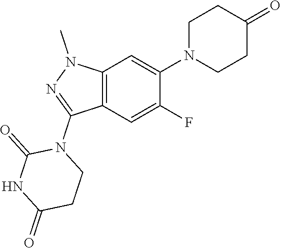 [SMILES: Cn1nc(N2CCC(=O)NC2=O)c2cc(F)c(N3CCC(=O)CC3)cc21] | LCMS (ESI, m/z): 360.14 [M + H]⁺ |
| | 1-(5-fluoro-1-methyl-6-(4- | |
| | oxopiperidin-1-yl)-1H-indazol-3- | |
| | yl)dihydropyrimidine-2,4(1H,3H)- | |
| | dione | |
| A37-b | 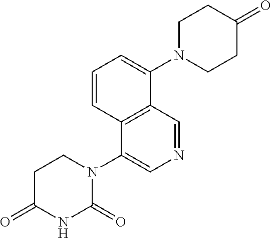 [SMILES: O=C1CCN(c2cccc3c(N4CCC(=O)NC4=O)cncc23)CC1] | LCMS (ESI, m/z): 339.14 [M + H]⁺ |
| | 1-(8-(4-oxopiperidin-1-yl)isoquinolin- | |
| | 4-yl)dihydropyrimidine-2,4(1H,3H)- | |
| | dione | |
| A37-c | 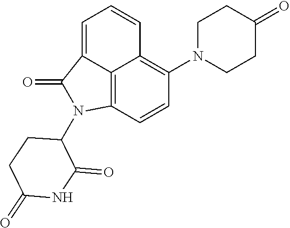 [SMILES: O=C1CCN(c2ccc3c4c(cccc24)C(=O)N3C2CCC(=O)NC2=O)CC1] | LCMS (ESI, m/z): 378.05 [M + H]⁺ |
| | 3-(2-oxo-6-(4-oxopiperidin-1- | |
| | yl)benzo[cd]indol-1(2H)-yl)piperidine- | |
| | 2,6-dione | |

### Intermediate A38: Synthesis of 2-(1-(3-fluoro-4-nitrophenyl)-4-hydroxypiperidin-4-yl)acetic acid

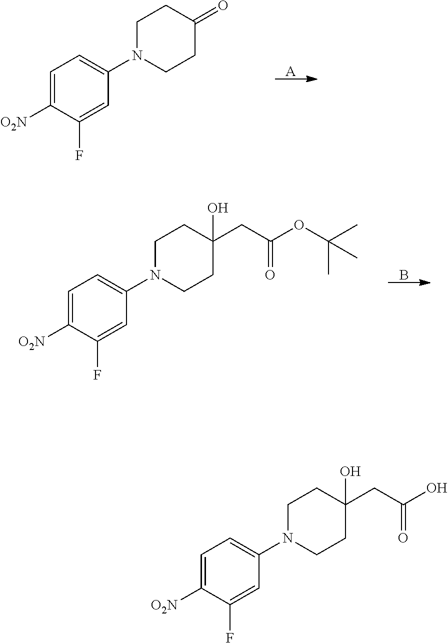

### Step A

A solution of tert-butyl acetate (0.63 g, 5.42 mmol, 1 equiv) in THF (10 mL) was treated with LDA (2 M in THF) (1.16 g, 10.8 mmol, 2 equiv) for 15 min at −78° C. under nitrogen atmosphere followed by the addition of 1-(3-fluoro-4-nitrophenyl)piperidin-4-one (1.49 g, 6.2 mmol, 1.15 equiv) portion-wise at −78° C. The mixture was stirred for 2 hours at room temperature under nitrogen atmosphere. The reaction was quenched with saturated aqueous NH₄Cl (10 mL) at room temperature. The mixture was extracted with EtOAc (3×10 mL). The combined organic layers were washed with water (3×10 mL), dried over anhydrous Na₂SO₄, filtered and concentrated. The residue was purified by silica gel column chromatography, eluting with PE/EtOAc (1:1) to afford tert-butyl 2-(1-(3-fluoro-4-nitrophenyl)-4-hydroxypiperidin-4-yl)acetate (958 mg, 59%) as a yellow solid. LCMS (ESI, m/z): 355.15 [M+H]⁺.

### Step B

A mixture of tert-butyl 2-(1-(3-fluoro-4-nitrophenyl)-4-hydroxypiperidin-4-yl)acetate (940 mg, 2.65 mmol, 1 equiv) in HCl in 1,4-dioxane (3 mL, 4 M) was stirred for 2 hours. The mixture was concentrated to dryness to afford 2-(1-(3-fluoro-4-nitrophenyl)-4-hydroxypiperidin-4-yl)acetic acid (716 mg, 91%) as a yellow solid that was used without further purification. LCMS (ESI, m/z): 299.15 [M+H]⁺.

### Intermediate A39: Synthesis of (3S)—N-(4-((2,6-dioxopiperidin-3-yl)amino)-2-fluorophenyl)pyrrolidine-3-carboxamide

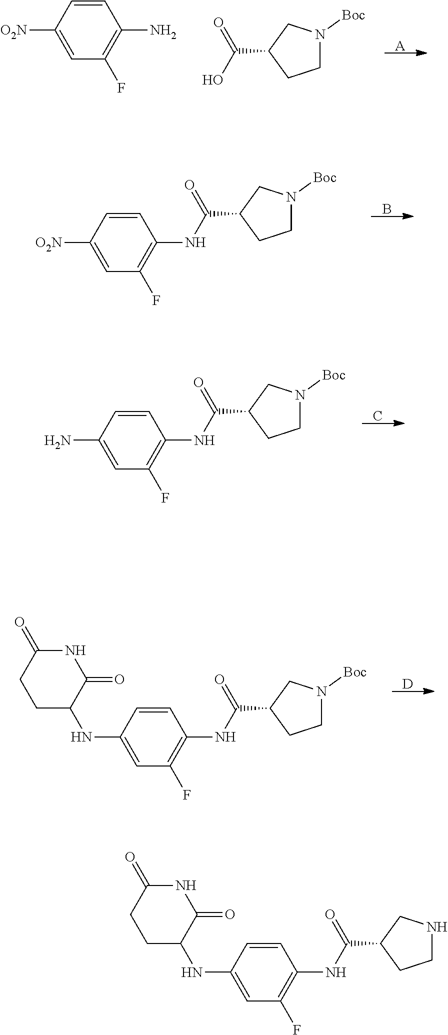

### Step A

A solution of 2-fluoro-4-nitroaniline (2.0 g, 13 mmol, 1 equiv) in DMF (10 mL) was treated with (3S)-1-(tert-butoxycarbonyl)pyrrolidine-3-carboxylic acid (2.76 g, 12.8 mmol, 1 equiv) and DIEA (4.97 g, 38.4 mmol, 3 equiv) followed by the dropwise addition of T₃P (16.3 g, 51.2 mmol, 4 equiv). The mixture was stirred overnight at 80° C. The mixture was purified by reversed-phase flash chromatography with the following conditions: column, C18 silica gel; mobile phase, MeCN in water (10 mmol/L NH₄HCO₃), 10% to 65% gradient in 35 min; detector, UV 254 nm to give tert-butyl (S)-3-((2-fluoro-4-nitrophenyl)carbamoyl)pyrrolidine-1-carboxylate (3.04 g, 67%) as an orange solid. LCMS (ESI, m/z): 352.10 [M−H]⁻.

### Step B

A solution of tert-butyl (3S)-3-[(2-fluoro-4-nitrophenyl)carbamoyl]pyrrolidine-1-carboxylate (3.02 g, 8.55 mmol, 1 equiv) in EtOH (28 mL) was treated with NH₄Cl (0.91 g, 17 mmol, 2 equiv) in water (7 mL) followed by the addition of Fe (2.39 g, 42.7 mmol, 5 equiv) portion-wise at 80° C. The mixture was stirred for 2 hours at 80° C. and then concentrated. The residue was purified by silica gel column chromatography, eluting with DCM/MeOH (93:7) to afford tert-butyl (S)-3-((4-amino-2-fluorophenyl)carbamoyl)pyrrolidine-1-carboxylate (2.6 g, 94%) as an orange solid. LCMS (ESI, m/z): 322.25 [M−H]⁻.

### Step C

A solution of tert-butyl (3S)-3-[(4-amino-2-fluorophenyl)carbamoyl]pyrrolidine-1-carboxylate (800 mg, 2.47 mmol, 1 equiv), 3-bromopiperidine-2,6-dione (475 mg, 2.47 mmol, 1 equiv) and NaHCO₃ (624 mg, 7.42 mmol, 3 equiv) in ACN (10 mL) was stirred overnight at 90° C. and then concentrated. The residue was purified by silica gel column chromatography, eluting with DCM/MeOH (19:1) to afford tert-butyl (3S)-3-((4-((2,6-dioxopiperidin-3-yl)amino)-2-fluorophenyl)carbamoyl)pyrrolidine-1-carboxylate (730 mg, 68%) as a green solid. LCMS (ESI, m/z): 433.30 [M−H]⁻.

### Step D

A solution of tert-butyl (3S)-3-((4-((2,6-dioxopiperidin-3-yl)amino)-2-fluorophenyl)carbamoyl)pyrrolidine-1-carboxylate (710 mg, 1.63 mmol, 1 equiv) in TFA (3 mL) and DCM (12 mL) was stirred for 30 mins. The mixture was concentrated to dryness to afford (3S)—N-(4-((2,6-dioxopiperidin-3-yl)amino)-2-fluorophenyl)pyrrolidine-3-carboxamide trifluoroacetate (1.3 g) as a dark blue solid. The product was used in the next step directly without further purification. LCMS (ESI, m/z): 335.15 [M+H]⁺.

Intermediates A39-a and A39-b were synthesized according to the procedure described for the synthesis of (3S)—N-(4-((2,6-dioxopiperidin-3-yl)amino)-2-fluorophenyl)pyrrolidine-3-carboxamide trifluoroacetate (Intermediate A39) using appropriate building blocks and modified reaction conditions (such as reagents, reagent ratio, temperature, and reaction time) and purification conditions as needed.

| Example No. | Structure | Analytical Data |
|---|---|---|
| A39-a | 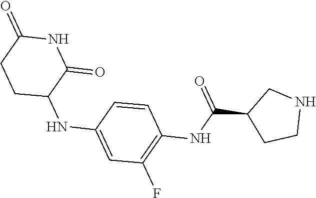 [SMILES: O=C1CCC(Nc2ccc(NC(=O)[C@@H]3CCNC3)c(F)c2)C(=O)N1] | LCMS (ESI, m/z): 335.20 [M + H]⁺ |
| | (3R)-N-(4-((2,6-dioxopiperidin-3- | |
| | yl)amino)-2-fluorophenyl)pyrrolidine- | |
| | 3-carboxamide | |
| A39-b | 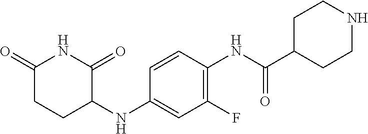 [SMILES: O=C1CCC(Nc2ccc(NC(=O)C3CCNCC3)c(F)c2)C(=O)N1] | LCMS (ESI, m/z): 349.20 [M + H]⁺ |
| | N-(4-((2,6-dioxopiperidin-3-yl)amino)- | |
| | 2-fluorophenyl)piperidine-4- | |
| | carboxamide | |

### Intermediate A40: Synthesis of N-(4-((2,6-dioxopiperidin-3-yl)amino)-2-fluorophenyl)-N-methylpiperidine-4-carboxamide

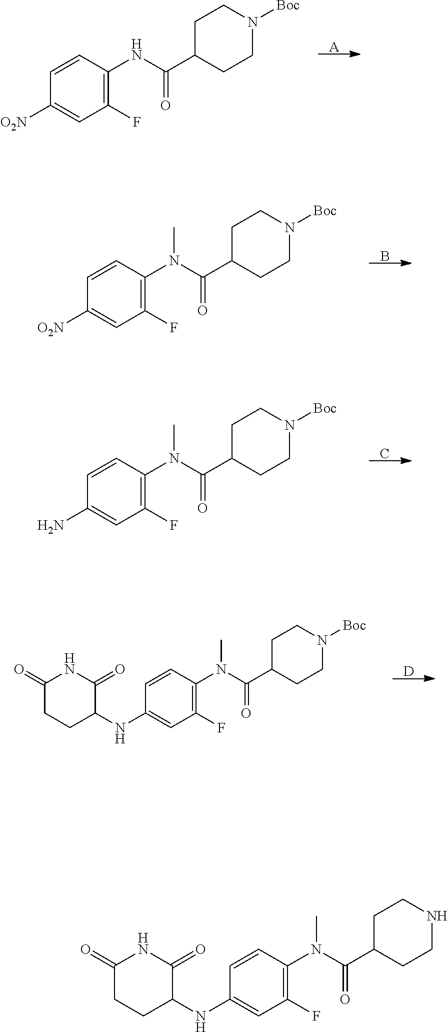

### Step A

A solution of tert-butyl 4-[(2-fluoro-4-nitrophenyl)carbamoyl]piperidine-1l-carboxylate (4 g, 10.9 mmol, 1 equiv) in DMF (10 mL) was treated with NaH (0.87 g, 21.8 mmol, 2 equiv, 60% dispersion in oil) and stirred for 15 min at 0° C. under nitrogen atmosphere. Methyl iodide (1.55 g, 10.9 mmol, 1 equiv) was added dropwise at 0° C. and the mixture was stirred at room temperature for 1 hour. The reaction was diluted with water and extracted with EtOAc (3×20 mL). The combined organic layers were washed with brine (3×10 mL) and dried over anhydrous Na₂SO₄. After filtration, the filtrate was concentrated under reduced pressure to afford tert-butyl 4-((2-fluoro-4-nitrophenyl)(methyl) carbamoyl)piperidine-1-carboxylate (2.79 g, 67%) as a white solid. LCMS (ESI, m/z): 382.2 [M+H]⁺.

### Step B

A solution of tert-butyl 4-[(2-fluoro-4-nitrophenyl)(methyl)carbamoyl]piperidine-1-carboxylate (2.79 g, 7.31 mmol, 1 equiv) and 10% Pd/C (0.78 g) in MeOH (10 mL) was stirred for 1 hour under 1 atmosphere of hydrogen. The mixture was filtered and washed with MeOH (3×10 mL). The filtrate was concentrated under reduced pressure to afford tert-butyl 4-((4-amino-2-fluorophenyl)(methyl)carbamoyl)piperidine-1-carboxylate (2.65 g) as a white solid that was used without further purification. LCMS (ESI, m/z): 352.2 [M+H]⁺.

### Step C

A solution of tert-butyl 4-[(4-amino-2-fluorophenyl)(methyl)carbamoyl]piperidine-1-carboxylate (2.0 g, 5.69 mmol, 1 equiv), 3-bromopiperidine-2,6-dione (1.31 g, 6.83 mmol, 1.2 equiv) and NaHCO₃ (1.43 g, 17.1 mmol, 3 equiv) in ACN (40 mL) was stirred for 3 days at 90° C. The solution was concentrated and the residue was purified by silica gel column chromatography, eluting with PE/EtOAc (1:1) to afford tert-butyl 4-((4-((2,6-dioxopiperidin-3-yl)amino)-2-fluorophenyl)(methyl)carbamoyl)piperidine-1-carboxylate. (2.5 g, 95%) as a white solid. LCMS (ESI, m/z): 463.3 [M+H]⁺.

### Step D

A solution of tert-butyl 4-((4-((2,6-dioxopiperidin-3-yl)amino)-2-fluorophenyl)(methyl)carbamoyl)piperidine-1-carboxylate (500 mg, 1.08 mmol, 1 equiv) in HCl in 1,4-dioxane (10 mL, 4M) was stirred for 1 hour. The mixture was concentrated to dryness to afford N-(4-((2,6-dioxopiperidin-3-yl)amino)-2-fluorophenyl)-N-methylpiperidine-4-carboxamide (440 mg) as a white solid. The crude product was used in the next step without further purification. LCMS (ESI, m/z): 363.3 [M+H]⁺.

### Intermediate A41: Synthesis of 1″-(2-fluoro-4-nitrophenyl)-4-(hydroxymethyl)-[1,4′:1′,4″-terpiperidin]-2-one

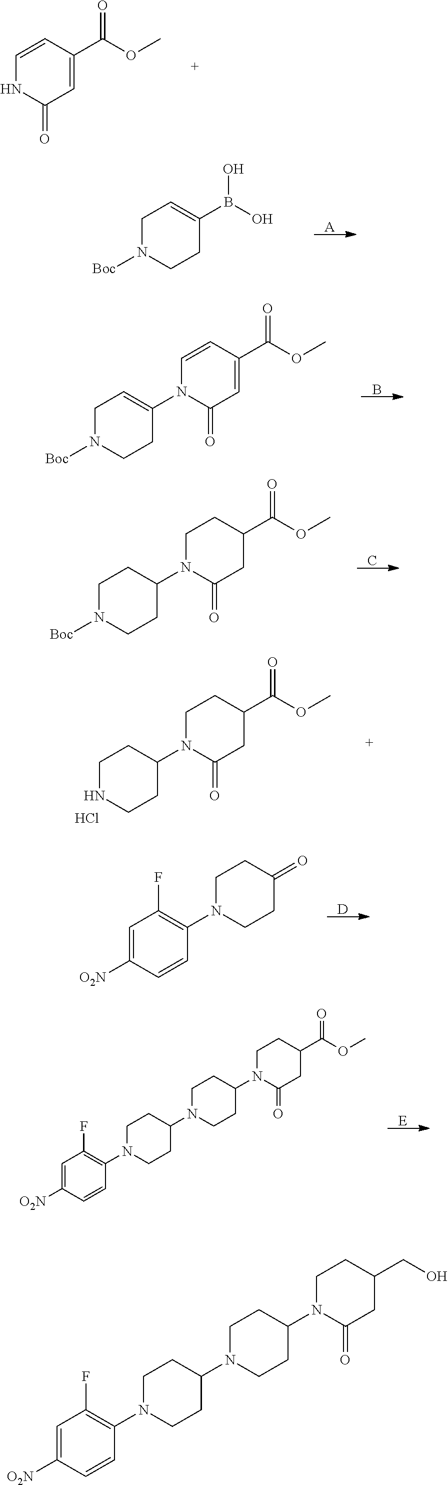

### Step A

A solution of methyl 2-oxo-1,2-dihydropyridine-4-carboxylate (1.35 g, 8.82 mmol, 1 equiv), (1-(tert-butoxycarbonyl)-1,2,3,6-tetrahydropyridin-4-yl)boronic acid (3.00 g, 13.2 mmol, 1.5 equiv), Cu(OAc)₂ (0.48 g, 2.7 mmol, 0.3 equiv), and pyridine (4.18 g, 52.9 mmol, 6 equiv) in THF (10 mL) was stirred for 15 hours at 50° C. in air. After concentration, the residue was purified by silica gel column chromatography, eluting with PE/EtOAc (2:3) to afford 1′-(tert-butyl) 4-methyl 2-oxo-3′,6′-dihydro-2H-[1,4′-bipyridine]-1′,4(2′H)-dicarboxylate (1.4 g, 47%) as a white solid. LCMS (ESI, m/z): 335.10 [M+H]⁺.

### Step B

A mixture of 1′-(tert-butyl) 4-methyl 2-oxo-3′,6′-dihydro-2H-[1,4′-bipyridine]-1′,4(2′H)-dicarboxylate (1.4 g, 4.2 mmol, 1 equiv) and 10% Pd/C (0.14 g) in EtOAc (5 mL) was stirred overnight under 1 atmosphere of hydrogen. The mixture was filtered and washed with EtOAc (3×5 mL). The filtrate was concentrated under reduced pressure to give 1′-(tert-butyl) 4-methyl 2-oxo-[1,4′-bipiperidine]-1′,4-dicarboxylate (1.2 g, 84%) as a white solid. LCMS (ESI, m/z): 339.20 [M−H]⁻.

### Step C

A mixture of 1′-(tert-butyl) 4-methyl 2-oxo-[1,4′-bipiperidine]-1′,4-dicarboxylate (1.2 g, 3.5 mmol, 1 equiv) in HCl in 1,4-dioxane (10 mL, 4M) was stirred for 4 hours. The mixture was concentrated to dryness to give methyl 2-oxo-[1,4′-bipiperidine]-4-carboxylate hydrochloride (1.4 g) as a white solid. The product was used in the next step without further purification. LCMS (ESI, m/z): 241.10 [M+H]⁺.

### Step D

A mixture of methyl 2-oxo-[1,4′-bipiperidine]-4-carboxylate hydrochloride (1.4 g, 5.8 mmol, 1 equiv), 1-(2-fluoro-4-nitrophenyl)piperidin-4-one (2.36 g, 9.90 mmol, 1.7 equiv), and STAB (2.47 g, 11.7 mmol, 2 equiv) in DCE (5 mL) was stirred for 2 days. The solution was concentrated and the residue was purified by reverse-phase flash chromatography with the following conditions: Column, C18 silica gel; Mobile phase, ACN in water (10 mmol/L NH₄HCO₃), 10% to 50% gradient in 15 min; detector, UV 254 nm to give methyl 1″-(2-fluoro-4-nitrophenyl)-2-oxo-[1,4′:1′,4″-terpiperidine]-4-carboxylate (900 mg, 33%) as a yellow solid. LCMS (ESI, m/z): 463.20 [M+H]⁺.

### Step E

A mixture of methyl 1″-(2-fluoro-4-nitrophenyl)-2-oxo-[1,4′:1′,4″-terpiperidine]-4-carboxylate (900 mg, 1.95 mmol, 1 equiv), NaBH₄ (110 mg, 2.92 mmol, 1.5 equiv), and CaCl₂) (432 mg, 3.89 mmol, 2 equiv) in EtOH (3 mL) was stirred overnight. The reaction was quenched with water at 0° C. The mixture was concentrated and the residue purified by silica gel column chromatography, eluting with CH₂Cl₂/MeOH (8:1) to afford 1″-(2-fluoro-4-nitrophenyl)-4-(hydroxymethyl)-[1,4′:1′,4″-terpiperidin]-2-one (400 mg, 47%) as a yellow solid. LCMS (ESI, m/z): 435.20 [M+H]⁺.

### Intermediate A42: Synthesis of 2-(1-(1-(2-fluoro-4-nitrophenyl)piperidin-4-yl)azetidin-3-yl)acetic acid

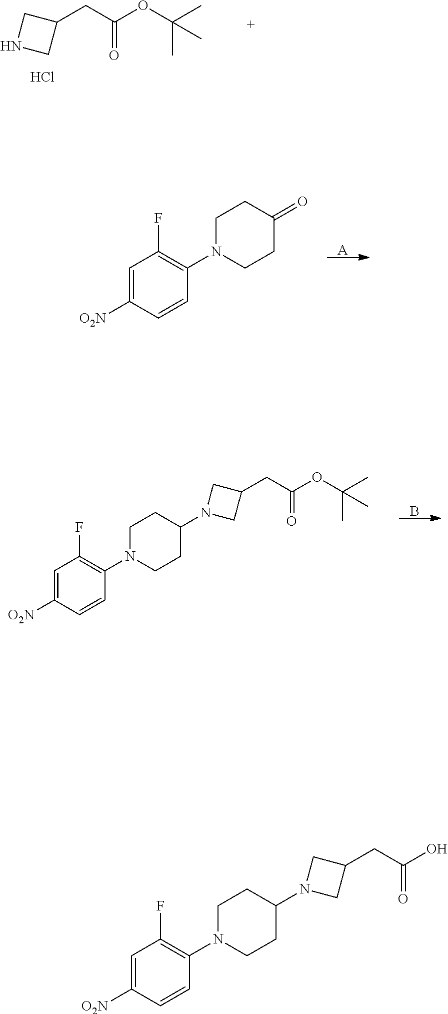

### Step A

A mixture of tert-butyl 2-(azetidin-3-yl)acetate hydrochloride (2.05 g, 9.86 mmol, 1 equiv) and 1-(2-fluoro-4-nitrophenyl)piperidin-4-one (3.52 g, 14.8 mmol, 1.5 equiv) in DCE (20 mL) was stirred for 1 hour. To the above mixture was added STAB (6.27 g, 29.6 mmol, 3 equiv) and the mixture stirred for 1 hour. After concentration the residue was purified by silica gel column chromatography, eluting with CH₂Cl₂/MeOH (4:1) to afford tert-butyl 2-(1-(1-(2-fluoro-4-nitrophenyl)piperidin-4-yl)azetidin-3-yl)acetate (2.4 g, 54%) as an orange oil. LCMS (ESI, m/z): 394.3 [M+H]⁺.

### Step B

A mixture of tert-butyl 2-(1-(1-(2-fluoro-4-nitrophenyl)piperidin-4-yl)azetidin-3-yl)acetate (2.38 g, 6.05 mmol, 1 equiv) and TFA (5 mL) in DCM (25 mL) was stirred for 2 hours. The mixture was concentrated to give 2-(1-(1-(2-fluoro-4-nitrophenyl)piperidin-4-yl)azetidin-3-yl)acetic acid (3.5 g) as an orange oil. The crude product was used in the next step without further purification. LCMS (ESI, m/z): 338.1 [M+H]⁺.

### Intermediate A43: Synthesis of 2-(4-(2-fluoro-4-nitrophenyl)-2-oxopiperazin-1-yl)acetaldehyde

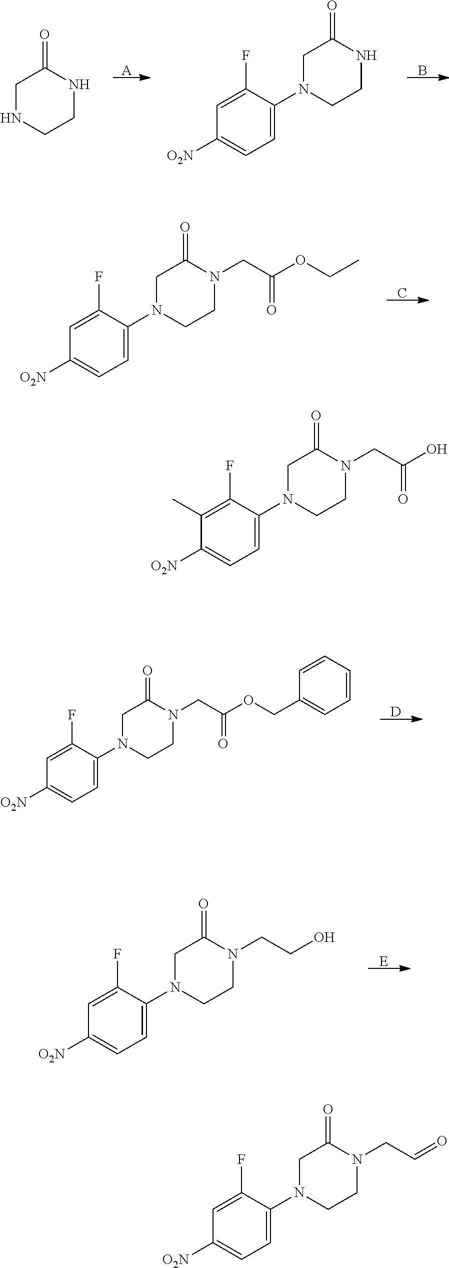

### Step A

A solution of piperazin-2-one (5.0 g, 50 mmol, 1 equiv), 1,2-difluoro-4-nitrobenzene (11.9 g, 74.9 mmol, 1.5 equiv), and NaHCO₃ (12.6 g, 150 mmol, 3 equiv) in ACN (60 mL) was stirred overnight at 80° C. The mixture was concentrated and the residue was purified by silica gel column chromatography, eluting with CH₂Cl₂/MeOH (12:1) to afford 4-(2-fluoro-4-nitrophenyl)piperazin-2-one (6.44 g, 54%) as a yellow solid. LCMS (ESI, m/z): 239.10 [M+H]⁺.

### Step B

To a solution of 4-(2-fluoro-4-nitrophenyl)piperazin-2-one (5.25 g, 21.9 mmol, 1 equiv) in THF (20 mL) was added NaH (1.05 g, 26.3 mmol, 1.2 equiv, 60% dispersion in oil) portion-wise at 0° C. The mixture was stirred for 20 min at 0° C. Ethyl bromoacetate (9.16 g, 54.9 mmol, 2.5 equiv) was added dropwise and the mixture stirred for 1 hour at 0° C. Water (10 mL) was added at 0° C. and then the mixture was extracted with EtOAc (3×30 mL). The combined organic layers were concentrated to dryness to afford ethyl 2-(4-(2-fluoro-4-nitrophenyl)-2-oxopiperazin-1-yl)acetate (8.37 g) as a yellow solid. The crude product was used in the next step without further purification. LCMS (ESI, m/z): 325.10 [M+H]⁺.

### Step C

A solution of ethyl 2-[4-(2-fluoro-4-nitrophenyl)-2-oxopiperazin-1-yl]acetate (8.37 g, 25.7 mmol, 1 equiv) and NaOH (3.09 g, 77.2 mmol, 3 equiv) in MeOH (10 mL) and water (40 mL) was stirred for 2 hours. The product was precipitated by adjusting to pH 6 with concentrated HCl. The precipitated solids were collected by filtration and washed with water (3×5 mL). The solid was dried under infrared light to afford 2-(4-(2-fluoro-4-nitrophenyl)-2-oxopiperazin-1-yl)acetic acid (6 g, 78%) as a yellow solid. LCMS (ESI, m/z): 297.10 [M+H]⁺.

### Step D

A solution of 2-(4-(2-fluoro-4-nitrophenyl)-2-oxopiperazin-1-yl)acetic acid (2.29 g, 7.70 mmol, 1 equiv), benzyl bromide (1.58 g, 9.25 mmol, 1.2 equiv), and K₂CO₃ (2.13 g, 15.4 mmol, 2 equiv) in DMF (10 mL) was stirred overnight. The product was precipitated by the addition of water. The precipitated solids were collected by filtration and washed with water (3×5 mL) to give benzyl 2-(4-(2-fluoro-4-nitrophenyl)-2-oxopiperazin-1-yl)acetate (1.98 g, 66%) as a yellow solid. LCMS (ESI, m/z): 387.10 [M+H]⁺.

### Step E

To a stirred solution of benzyl 2-[4-(2-fluoro-4-nitrophenyl)-2-oxopiperazin-1-yl]acetate (516 mg, 1.33 mmol, 1 equiv) and LiCl (113 mg, 2.66 mmol, 2 equiv) in EtOH (6 mL) and THF (6 mL) was added NaBH₄ (101 mg, 2.66 mmol, 2 equiv) portion-wise at 0° C. The mixture was stirred for 1 hour at room temperature. The solution was cooled to 0° C. and water was added. The mixture was concentrated and the residue was purified by silica gel column chromatography, eluting with CH₂Cl₂/MeOH (10:1) to afford 4-(2-fluoro-4-nitrophenyl)-1-(2-hydroxyethyl)piperazin-2-one (278 mg, 74%) as a yellow solid. LCMS (ESI, m/z): 283.10 [M+H]⁺.

### Step F

A solution of 4-(2-fluoro-4-nitrophenyl)-1-(2-hydroxyethyl)piperazin-2-one (278 mg, 0.981 mmol, 1 equiv) and Dess-Martin periodinane (624 mg, 1.47 mmol, 1.5 equiv) in DCM (5 mL) was stirred for 3 hours. The solution was cooled to 0° C. and saturated aqueous NaHCO₃ (10 mL) was added. The mixture was extracted with CH₂Cl₂ (3×15 mL) and the combined organic layers were concentrated under reduced pressure. The residue was purified by silica gel column chromatography, eluting with PE/EtOAc (1:1) to afford 2-(4-(2-fluoro-4-nitrophenyl)-2-oxopiperazin-1-yl)acetaldehyde (153 mg, 55%) as a yellow oil. LCMS (ESI, m/z): 281.10 [M+H]⁺.

### Intermediate A44: Synthesis of 1-(1-(2-fluoro-4-nitrophenyl)piperidin-4-yl)azetidine-3-carboxylic acid

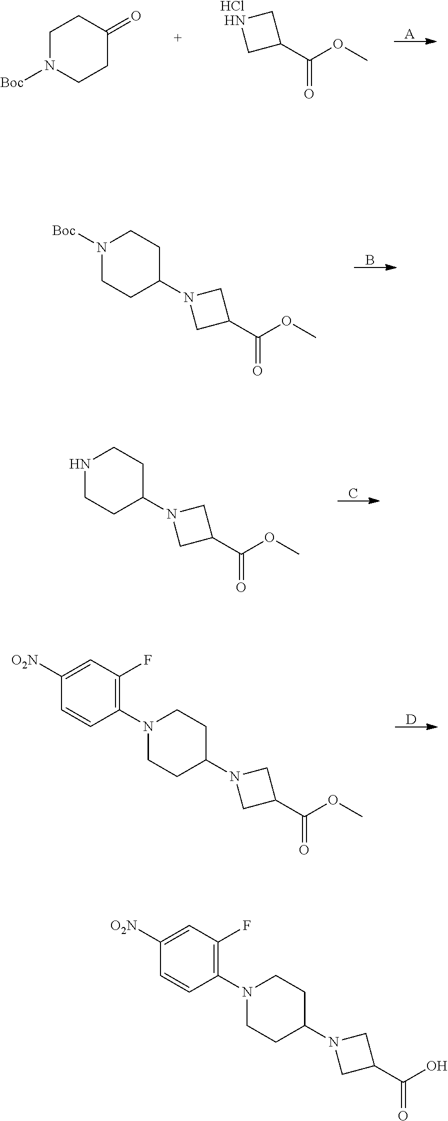

### Step A

A solution of tert-butyl 4-oxopiperidine-1-carboxylate (1.5 g, 7.5 mmol, 1 equiv) in DCE (15 mL) was treated with methyl azetidine-3-carboxylate hydrochloride (1.30 g, 11.3 mmol, 1.5 equiv) and STAB (3.19 g, 15.1 mmol, 2 equiv) for 3 hours at 40° C. The mixture was concentrated and the residue was purified by silica gel column chromatography, eluting with PE/EtOAc (55:45) to afford tert-butyl 4-(3-(methoxycarbonyl)azetidin-1-yl)piperidine-1-carboxylate (2.21 g, 98%) as a white solid. LCMS (ESI, m/z): 299.10 [M+H]⁺.

### Step B

A solution of tert-butyl 4-(3-(methoxycarbonyl)azetidin-1-yl)piperidine-1-carboxylate (2.4 g, 8.0 mmol, 1 equiv) in HCl in 1,4-dioxane (25 mL, 4 M) was stirred for 1 hour. The mixture was concentrated to dryness to afford methyl 1-(piperidin-4-yl)azetidine-3-carboxylate (2.05 g) as a white solid. The crude product was used in the next step without further purification. LCMS (ESI, m/z): 199.15 [M+H]⁺.

### Step C

A solution of methyl 1-(piperidin-4-yl)azetidine-3-carboxylate (2.05 g, 10.1 mmol, 1 equiv) in ACN (15 mL) was treated with 1,2-difluoro-4-nitrobenzene (3.21 g, 20.2 mmol, 2 equiv) and NaHCO₃ (2.54 g, 30.3 mmol, 3 equiv) for 2 hours at 80° C. The mixture was concentrated and the residue was purified by silica gel column chromatography, eluted with CH₂Cl₂/MeOH (95:5) to afford methyl 1-(1-(2-fluoro-4-nitrophenyl)piperidin-4-yl)azetidine-3-carboxylate (2.27 g, 67%) as a yellow solid. LCMS (ESI, m/z): 338.15 [M+H]⁺.

### Step D

A solution of methyl 1-(1-(2-fluoro-4-nitrophenyl)piperidin-4-yl)azetidine-3-carboxylate (2.25 g, 6.66 mmol, 1 equiv) in MeOH (3 mL) was treated with NaOH (1.07 g, 26.6 mmol, 4 equiv) and water (15 mL) for 1 hour. The mixture was adjusted to pH 6 with aqueous HCl. The precipitated solids were collected by filtration to give 1-(1-(2-fluoro-4-nitrophenyl)piperidin-4-yl)azetidine-3-carboxylic acid (2.13 g, 99%) as a yellow solid. LCMS (ESI, m/z): 324.15 [M+H]⁺.

### Intermediate A45: Synthesis of 1-(1-methyl-6-(4-oxopiperidin-1-yl)-1H-indazol-3-yl)dihydropyrimidine-2,4(1H,3H)-dione

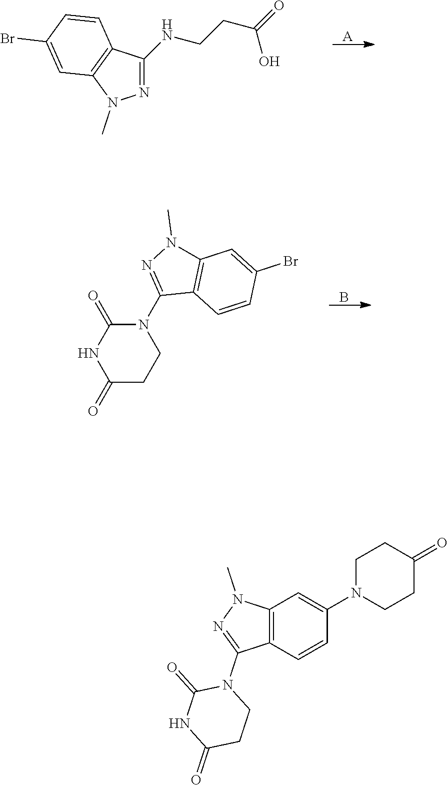

### Step A

A mixture of 3-((6-bromo-1-methyl-1H-indazol-3-yl)amino)propanoic acid (1.6 g, 5.4 mmol, 1 equiv) and NaOCN (697 mg, 10.7 mmol, 2 equiv) in HOAc (19 mL) was stirred overnight at 60° C. under nitrogen. To the mixture was added HCl (19 mL, 2 M) dropwise. The mixture was stirred for 3 hours at 60° C. The mixture was cooled to room temperature and concentrated. The residue was purified by trituration with water (130 mL) to give 1-(6-bromo-1-methyl-1H-indazol-3-yl)dihydropyrimidine-2,4(1H,3H)-dione (1.19 g, 66%) as an off-white solid after filtration. LCMS (ESI, m/z): 324.95 [M+H]⁺.

### Step B

A mixture of 1-(6-bromo-1-methyl-1H-indazol-3-yl)dihydropyrimidine-2,4(1H,3H)-dione (360 mg, 1.11 mmol, 1 equiv), piperidin-4-one hydrochloride (151 mg, 1.11 mmol, 1 equiv), Pd PEPPSI IPentCl (94 mg, 0.11 mmol, 0.1 equiv) and Cs₂CO₃ (726 mg, 2.23 mmol, 2 equiv) in 1,4-dioxane (4 mL) was stirred overnight at 85° C. under nitrogen atmosphere. The mixture was allowed to cool to room temperature and concentrated under reduced pressure. The residue was purified by reverse-phase flash chromatography with the following conditions: column, C18 silica gel; mobile phase, MeCN in water (10 mmol/L NH₄HCO₃), 10% to 50% gradient in 20 min; detector, UV 254 nm to give 1-(1-methyl-6-(4-oxopiperidin-1-yl)-1H-indazol-3-yl)dihydropyrimidine-2,4(1H,3H)-dione (200 mg, 47%) as an orange solid. LCMS (ESI, m/z): 342.10 [M+H]⁺.

Intermediate A45-a was synthesized according to the procedure described for the synthesis of 1-(1-methyl-6-(4-oxopiperidin-1-yl)-1H-indazol-3-yl)dihydropyrimidine-2,4(1H,3H)-dione (Intermediate A45) using appropriate building blocks and modified reaction conditions (such as reagents, reagent ratio, temperature, and reaction time) and purification conditions as needed.

| Example No. | Structure | Analytical Data |
|---|---|---|
| A45-a | 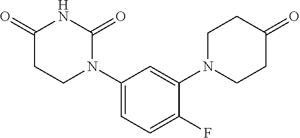 [SMILES: O=C1CCN(c2cc(N3CCC(=O)NC3=O)ccc2F)CC1] | LCMS (ESI, m/z): 306.10 [M + H]⁺ |
| | 1-(4-fluoro-3-(4-oxopiperidin-1- | |
| | yl)phenyl)dihydropyrimidine-2,4(1H,3H)- | |
| | dione | |

### Intermediate A46: Synthesis of 1-(5-fluoro-1-methyl-6-(piperidin-4-yl)-1H-indazol-3-yl)dihydropyrimidine-2,4(1H,3H)-dione hydrochloride

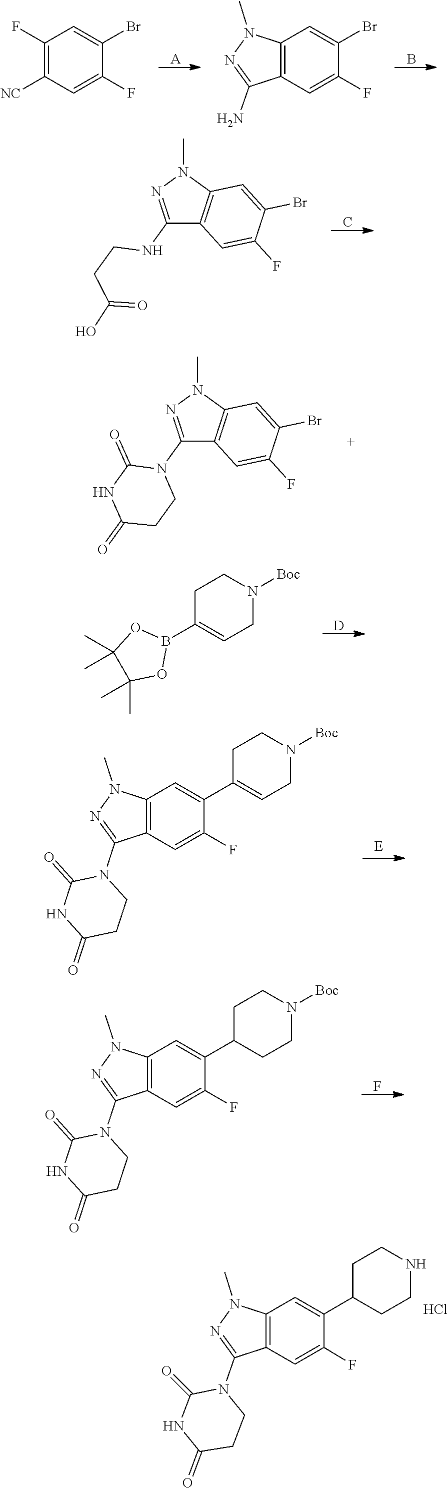

### Step A

A solution of 4-bromo-2,5-difluorobenzonitrile (3.0 g, 14 mmol, 1 equiv), methylhydrazine sulfate (9.92 g, 68.8 mmol, 5 equiv) and K₂CO₃ (9.51 g, 68.8 mmol, 5 equiv) in n-BuOH (100 mL) was stirred for 5 hours at 100° C. The mixture was concentrated and the residue was purified by silica gel column chromatography, eluting with PE/EtOAc (1:1) to afford 6-bromo-5-fluoro-1-methyl-1H-indazol-3-amine (1.7 g, 51%) as a yellow solid. LCMS (ESI, m/z): 243.98 [M+H]⁺.

### Step B

A solution of 6-bromo-5-fluoro-1-methyl-1H-indazol-3-amine (1.7 g, 6.97 mmol, 1 equiv) and acrylic acid (3.01 g, 41.8 mmol, 6 equiv) in toluene (50 mL) was stirred overnight. The mixture was concentrated and the residue diluted with water (5 mL). The mixture was neutralized to pH 7 with saturated aqueous NaHCO₃. The mixture was concentrated under reduced pressure and purified by reverse-phase flash chromatography with the following conditions: column, C18 silica gel; mobile phase, MeCN in water (10 mmol/L NH₄HCO₃), 10% to 20% gradient in 10 min; detector, UV 254 nm to give 3-((6-bromo-5-fluoro-1-methyl-1H-indazol-3-yl)amino)propanoic acid (998 mg, 45%) as a yellow solid. LCMS (ESI, m/z): 316.00 [M+H]⁺.

### Step C

A solution of 3-((6-bromo-5-fluoro-1-methyl-1H-indazol-3-yl)amino)propanoic acid (988 mg, 3.13 mmol, 1 equiv) and NaOCN (406 mg, 6.25 mmol, 2 equiv) in HOAc (6 mL) was stirred overnight at 60° C. HCl (6 mL, 2 M) was then added dropwise over 1 min at room temperature. The mixture was stirred for 3 hour at 60° C. The solution was diluted with water (15 mL) and concentrated to remove volatiles. The precipitated solids were collected by filtration and washed with water (30 mL) to afford 1-(6-bromo-5-fluoro-1-methyl-1H-indazol-3-yl)dihydropyrimidine-2,4(1H,3H)-dione (688 mg, 65%) as an off-white solid. LCMS (ESI, m/z): 341.00 [M+H]⁺.

### Step D

A solution of 1-(6-bromo-5-fluoro-1-methyl-1H-indazol-3-yl)dihydropyrimidine-2,4(1H,3H)-dione (688 mg, 2.02 mmol, 1 equiv), tert-butyl 4-(4,4,5,5-tetramethyl-1,3,2-dioxaborolan-2-yl)-3,6-dihydropyridine-1(2H)-carboxylate (748 mg, 2.42 mmol, 1.2 equiv), Pd(dppf)Cl₂·CH₂Cl₂ (164 mg, 0.202 mmol, 0.1 equiv), K₂CO₃ (558 mg, 4.03 mmol, 2 equiv) in water (2 mL) and dioxane (20 mL) was stirred for 1 hour at 80° C. The mixture was concentrated and the residue purified by silica gel column chromatography, eluting with PE/EtOAc (1:99) to afford tert-butyl 4-(3-(2,4-dioxotetrahydropyrimidin-1(2H)-yl)-5-fluoro-1-methyl-1H-indazol-6-yl)-3,6-dihydropyridine-1(2H)-carboxylate (715 mg, 80%) as an off-white solid. LCMS (ESI, m/z): 444.20 [M+H]⁺.

### Step E

A solution of tert-butyl 4-(3-(2,4-dioxotetrahydropyrimidin-1(2H)-yl)-5-fluoro-1-methyl-1H-indazol-6-yl)-3,6-dihydropyridine-1(2H)-carboxylate (705 mg, 1.59 mmol, 1 equiv) and 10% Pd/C (508 mg) in EtOAc (20 mL) was stirred for 30 min under hydrogen atmosphere. After filtration, the filter cake was washed with EtOAc (50 mL). The filtrate was concentrated to afford tert-butyl 4-(3-(2,4-dioxotetrahydropyrimidin-1(2H)-yl)-5-fluoro-1-methyl-1H-indazol-6-yl)piperidine-1-carboxylate (320 mg, 45%) as an off-white solid. LCMS (ESI, m/z): 446.21 [M+H]⁺.

### Step F

A solution of tert-butyl 4-(3-(2,4-dioxotetrahydropyrimidin-1(2H)-yl)-5-fluoro-1-methyl-1H-indazol-6-yl)piperidine-1-carboxylate (310 mg, 0.70 mmol, 1 equiv) in HCl in 1,4-dioxane (10 mL, 4 M) was stirred for 1 hour. The mixture was concentrated to dryness to afford 1-(5-fluoro-1-methyl-6-(piperidin-4-yl)-1H-indazol-3-yl)dihydropyrimidine-2,4(1H,3H)-dione hydrochloride (255 mg, 96%) as an off-white solid. The crude product was used in the next step directly without further purification. LCMS (ESI, m/z): 346.10 [M+H]⁺.

### Intermediate A47: Synthesis of 1-(7-(piperazin-1-yl)imidazo[1,2-a]pyridin-3-yl)dihydropyrimidine-2,4(1H,3H)-dione

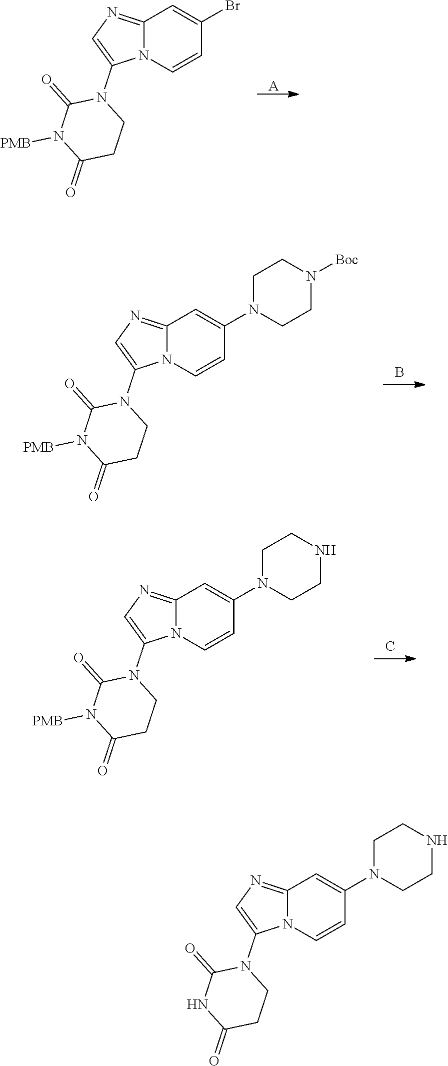

### Step A

A solution of 1-(7-bromoimidazo[1,2-a]pyridin-3-yl)-3-(4-methoxybenzyl)-dihydropyrimidine-2,4(1H,3H)-dione (500 mg, 1.17 mmol, 1 equiv), tert-butyl piperazine-1-carboxylate (325 mg, 1.75 mmol, 1.5 equiv), and Cs₂CO₃ (38.0 mg, 0.117 mmol, 0.1 equiv) in dioxane (5 mL) was treated with Pd PEPPSI IPentCl (98.0 mg, 0.117 mmol, 0.1 equiv) under nitrogen atmosphere. The mixture was stirred for 4 hours at 90° C. After concentration, the crude product was purified by silica gel chromatography eluting with DCM/MeOH (95:5) to afford tert-butyl 4-(3-(3-(4-methoxybenzyl)-2,4-dioxotetrahydropyrimidin-1(2H)-yl)imidazo[1,2-a]pyridin-7-yl)piperazine-1-carboxylate (576 mg, 93%) as a yellow-green solid. LCMS (ESI, m/z): 535.25 [M+H]⁺.

### Step B

A solution of tert-butyl 4-(3-(3-(4-methoxybenzyl)-2,4-dioxotetrahydropyrimidin-1(2H)-yl)imidazo[1,2-a]pyridin-7-yl)piperazine-1-carboxylate (250 mg, 0.468 mmol, 1 equiv) and TFA (10 mL) in DCM (30 mL) was stirred for 1 hour. The mixture was concentrated to dryness to afford 3-(4-methoxybenzyl)-1-(7-(piperazin-1-yl)imidazo[1,2-a]pyridin-3-yl)dihydropyrimidine-2,4(1H,3H)-dione (230 mg) as a dark-green solid. The crude product was used in the next step without further purification. LCMS (ESI, m/z): 435.30 [M+H]⁺.

### Step C

A solution of 3-(4-methoxybenzyl)-1-(7-(piperazin-1-yl)imidazo[1,2-a]pyridin-3-yl)dihydropyrimidine-2,4(1H,3H)-dione (230 mg, 0.529 mmol, 1 equiv) and TfOH (5 mL) in TFA (25 mL) was stirred for 3 hours. The solution was adjusted to pH 7 with saturated aqueous NaHCO₃. The mixture was concentrated to dryness and the residue was purified by silica gel column eluting with DCM/MeOH (90:10) to afford 1-(7-(piperazin-1-yl)imidazo[1,2-a]pyridin-3-yl)dihydropyrimidine-2,4(1H,3H)-dione (170 mg, crude) as a dark-green solid. LCMS (ESI, m/z): 315.05[M+H]⁺.

### Intermediate A48: Synthesis of 1-(8-(piperazin-1-yl)isoquinolin-4-yl)dihydropyrimidine-2,4(1H,3H)-dione hydrochloride

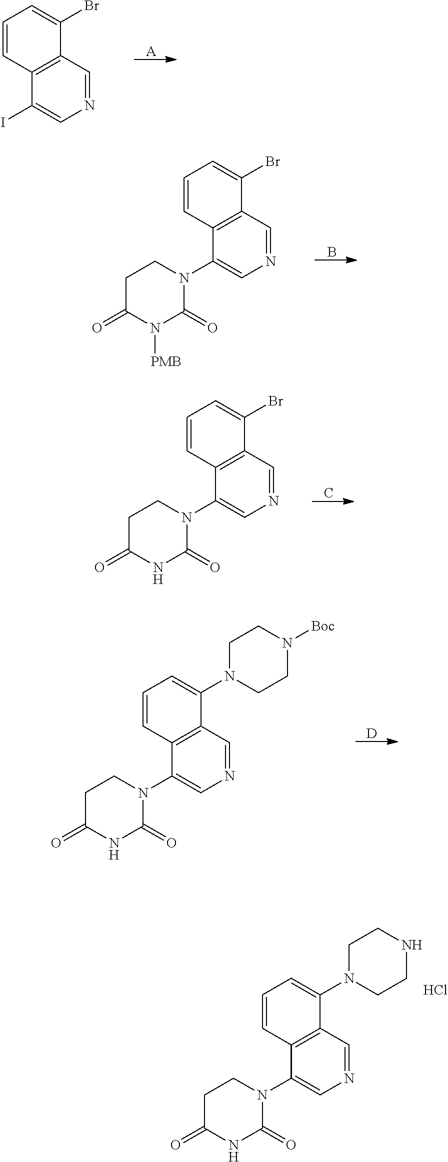

### Step A

A solution of 8-bromo-4-iodoisoquinoline (1.00 g, 2.99 mmol, 1 equiv), 3-(4-methoxybenzyl)dihydropyrimidine-2,4(1H,3H)-dione (0.91 g, 3.89 mmol, 1.3 equiv), (1R,2R)—N1,N2-dimethylcyclohexane-1,2-diamine (0.21 g, 1.50 mmol, 0.5 equiv), Cs₂CO₃ (1.95 g, 6.0 mmol, 2 equiv) and CuI (0.23 g, 1.20 mmol, 0.4 equiv) in dioxane (6 mL) was stirred overnight at 65° C. under nitrogen atmosphere. The mixture was concentrated and the residue was purified by silica gel column chromatography, eluting with PE/EtOAc (45:55) to afford 1-(8-bromoisoquinolin-4-yl)-3-(4-methoxybenzyl)dihydropyrimidine-2,4(1H,3H)-dione (730 mg, 55%) as a yellow solid. LCMS (ESI, m/z): 440.05 [M+H]⁺.

### Step B

A solution of 1-(8-bromoisoquinolin-4-yl)-3-(4-methoxybenzyl)dihydropyrimidine-2,4(1H,3H)-dione (1.6 g, 3.6 mmol, 1 equiv) in TFA (5 mL) and TfOH (1 mL) was stirred for 4 hours. The solution was diluted with EtOAc (4 mL). The mixture was basified to pH 8 with TEA. The precipitated solids were collected by filtration and washed with water (3×5 mL) to give 1-(8-bromoisoquinolin-4-yl)dihydropyrimidine-2,4(1H,3H)-dione (1.5 g) as a yellow solid that was used without further purification. LCMS (ESI, m/z): 320.00 [M+H]⁺.

### Step C

A solution of 1-(8-bromoisoquinolin-4-yl)dihydropyrimidine-2,4(1H,3H)-dione (0.500 g, 1.56 mmol, 1 equiv), tert-butyl piperazine-1-carboxylate (436 mg, 2.34 mmol, 1.5 equiv), Pd PEPPSI IPentCl (131 mg, 0.156 mmol, 0.1 equiv) and Cs₂CO₃ (1.02 g, 3.12 mmol, 2 equiv) in dioxane (4 mL) was stirred for 3 hours at 85° C. under nitrogen atmosphere. The mixture was concentrated and the residue was purified by silica gel column chromatography, eluting with DCM/EtOH (92:8) to afford tert-butyl 4-(4-(2,4-dioxotetrahydropyrimidin-1(2H)-yl)isoquinolin-8-yl)piperazine-1-carboxylate (377 mg, 57%) as a yellow solid. LCMS (ESI, m/z): 426.21 [M+H]⁺.

### Step D

A solution of tert-butyl 4-(4-(2,4-dioxotetrahydropyrimidin-1(2H)-yl)isoquinolin-8-yl)piperazine-1-carboxylate (367 mg, 0.863 mmol, 1 equiv) in HCl in 1,4-dioxane (5 mL, 4 M) was stirred for 1 hour. The mixture was concentrated to dryness to give 1-(8-(piperazin-1-yl)isoquinolin-4-yl)dihydropyrimidine-2,4(1H,3H)-dione hydrochloride (400 mg) as a yellow solid. The crude product was used in the next step directly without further purification. LCMS (ESI, m/z): 326.15 [M+H]⁺.

### Intermediate A49: Synthesis of 2-(9-(2-fluoro-4-nitrophenyl)-3,9-diazaspiro[5.5]undecan-3-yl)ethan-1-ol

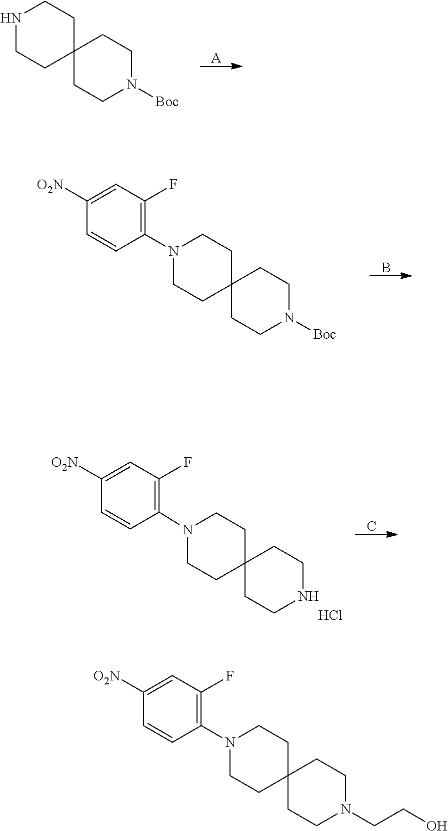

### Step A

A solution of tert-butyl 3,9-diazaspiro[5.5]undecane-3-carboxylate (5.00 g, 19.7 mmol, 1.0 equiv), 1,2-difluoro-4-nitrobenzene (3.44 g, 21.6 mmol, 1.1 equiv) and TEA (5.97 g, 59.0 mmol, 3.0 equiv) in DMF (20 mL) was stirred for 4 hours. The mixture was diluted with water. The precipitated solid were collected by filtration and dried to afford tert-butyl 9-(2-fluoro-4-nitrophenyl)-3,9-diazaspiro[5.5]undecane-3-carboxylate (6.4 g, 83%) as a yellow solid. The crude product was used in the next step without further purification. LCMS (ESI, m/z): 394.30 [M+H]⁺.

### Step B

A solution of tert-butyl 9-(2-fluoro-4-nitrophenyl)-3,9-diazaspiro[5.5]undecane-3-carboxylate (6.4 g, 16 mmol, 1 equiv) in HCl in 1,4-dioxane (50 mL, 4 M) was stirred for 2 hours. The mixture was concentrated under vacuum to afford 3-(2-fluoro-4-nitrophenyl)-3,9-diazaspiro[5.5]undecane (7 g) as a yellow solid. The crude product was used in the next step without further purification. LCMS (ESI, m/z): 294.20 [M+H]⁺.

### Step C

A solution of 3-(2-fluoro-4-nitrophenyl)-3,9-diazaspiro[5.5]undecane (6.0 g, 18 mmol, 1 equiv), 2-bromoethan-1-ol (2.27 g, 18.2 mmol, 1 equiv) and K₂CO₃ (7.54 g, 54.6 mmol, 3 equiv) in DMF (20 mL) was stirred overnight at 80° C. The reaction was diluted with water, extracted with ethyl acetate (3×100 mL) and washed with brine. The organics were dried over anhydrous sodium sulfate and concentrated under vacuum. The crude product was purified by silica gel chromatography eluting with CH₂Cl₂: MeOH (96:4) to afford 2-(9-(2-fluoro-4-nitrophenyl)-3,9-diazaspiro[5.5]undecan-3-yl)ethan-1-ol (1.8 g, 29%) as a yellow oil. LCMS (ESI, m/z): 338.30 [M+H]⁺.

### Intermediate A50: Synthesis of 1-(7-(piperidin-4-yl)imidazo[1,2-a]pyridin-3-yl)dihydropyrimidine-2,4(1H,3H)-dione

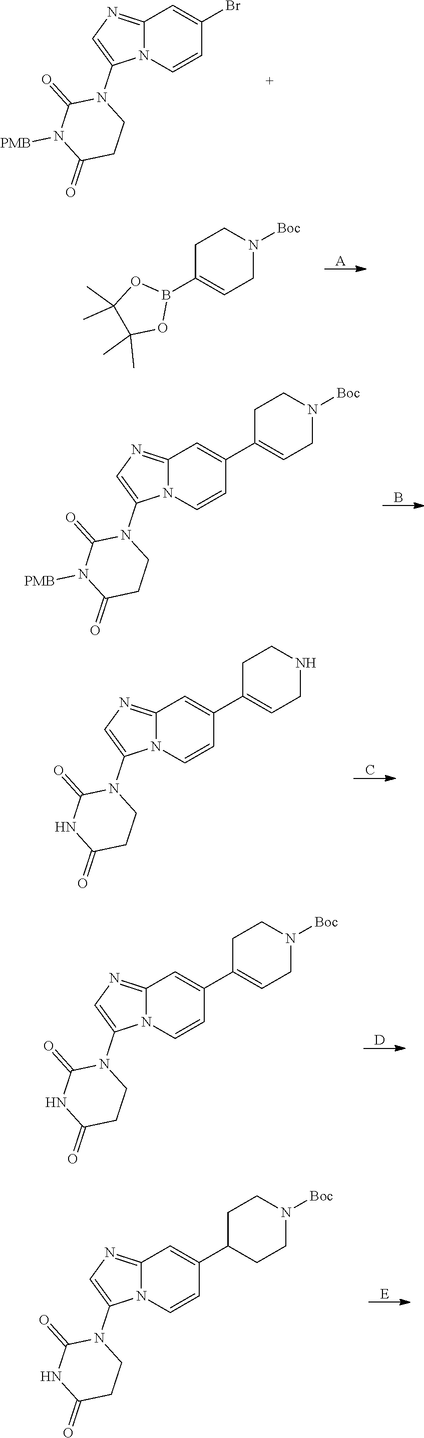

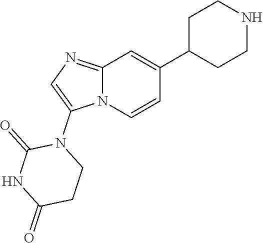 [SMILES: O=C1CCN(c2cnc3cc(C4CCNCC4)ccn23)C(=O)N1]

### Step A

A solution of 1-(7-bromoimidazo[1,2-a]pyridin-3-yl)-3-(4-methoxybenzyl)dihydropyrimidine-2,4(1H,3H)-dione (533 mg, 1.24 mmol, 1 equiv), tert-butyl 4-(4,4,5,5-tetramethyl-1,3,2-dioxaborolan-2-yl)-3,6-dihydro-2H-pyridine-1-carboxylate (768 mg, 2.48 mmol, 2 equiv), Pd(dppf)Cl₂·CH₂Cl₂ (101 mg, 0.124 mmol, 0.1 equiv) and K₂CO₃ (343 mg, 2.48 mmol, 2 equiv) in dioxane (10 mL) and water (1 mL) was stirred overnight at 85° C. under nitrogen atmosphere. The mixture was concentrated and the residue was purified by reverse-phase flash chromatography with the following conditions: column, C18 silica gel; mobile phase MeCN in water (10 mmol/L NH₄HCO₃), 10% to 65% gradient in 40 min; detector, UV 254 nm to give tert-butyl 4-(3-(3-(4-methoxybenzyl)-2,4-dioxotetrahydropyrimidin-1(2H)-yl)imidazo[1,2-a]pyridin-7-yl)-3,6-dihydropyridine-1(2H)-carboxylate (572 mg, 87%) as a brown solid. LCMS (ESI, m/z): 532.30 [M+H]⁺.

### Step B

A solution of tert-butyl 4-(3-(3-(4-methoxybenzyl)-2,4-dioxotetrahydropyrimidin-1(2H)-yl)imidazo[1,2-a]pyridin-7-yl)-3,6-dihydropyridine-1(2H)-carboxylate (520 mg, 0.978 mmol, 1 equiv) and methanesulfonic acid (5 mL) in toluene (10 mL) was stirred overnight at 100° C. The mixture was adjusted to pH 8 with TEA. The mixture was concentrated to dryness to afford 1-(7-(1,2,3,6-tetrahydropyridin-4-yl)imidazo[1,2-a]pyridin-3-yl)dihydropyrimidine-2,4(1H,3H)-dione (410 mg) as a brown-yellow oil. The crude product was used in next step without further purification. LCMS (ESI, m/z): 312.10 [M+H]⁺.

### Step C

A solution of 1-[7-(1,2,3,6-tetrahydropyridin-4-yl)imidazo[1,2-a]pyridin-3-yl]-1,3-diazinane-2,4-dione (400 mg, 1.29 mmol, 1 equiv), Boc₂O (280 mg, 1.29 mmol, 1 equiv) and TEA (260 mg, 2.57 mmol, 2 equiv) in DCM (10 mL) was stirred overnight. The mixture was adjusted to pH 8 with saturated aqueous NaHCO₃. The mixture was extracted with DCM (3×100 mL). The organics were dried and concentrated. The residue was purified by reverse-phase flash chromatography with the following conditions: column, C18 silica gel; mobile phase, MeCN in water (10 mmol/L NH₄HCO₃), 10% to 50% gradient in 35 min; detector, UV 254 nm to give tert-butyl 4-(3-(2,4-dioxotetrahydropyrimidin-1(2H)-yl)imidazo[1,2-a]pyridin-7-yl)-3,6-dihydropyridine-1(2H)-carboxylate (211 mg, 40%) as a yellow solid. LCMS (ESI, m/z): 412.30 [M+H]⁺.

### Step D

A solution of tert-butyl 4-[3-(2,4-dioxo-1,3-diazinan-1-yl)imidazo[1,2-a]pyridin-7-yl]-3,6-dihydro-2H-pyridine-1-carboxylate (201 mg, 0.489 mmol, 1 equiv) and 10% Pd/C (300 mg) in EtOAc (25 mL) was stirred for 30 min under hydrogen atmosphere. The mixture was filtered, and the filter cake was washed with EtOAc (6×100 mL). The filtrate was concentrated to afford tert-butyl 4-(3-(2,4-dioxotetrahydropyrimidin-1(2H)-yl)imidazo[1,2-a]pyridin-7-yl)piperidine-1-carboxylate (190 mg, 94%) as a white solid. LCMS (ESI, m/z): 414.30 [M+H]⁺.

### Step E

A solution of tert-butyl 4-[3-(2,4-dioxo-1,3-diazinan-1-yl)imidazo[1,2-a]pyridin-7-yl]piperidine-1-carboxylate (180 mg, 0.435 mmol, 1 equiv) in HCl in 1,4-dioxane (4 M, 10 mL) was stirred for 1 hour. The mixture was concentrated to dryness to afford crude 1-(7-(piperidin-4-yl)imidazo[1,2-a]pyridin-3-yl)dihydropyrimidine-2,4(1H,3H)-dione (198 mg) as a yellow solid. The crude product was used in next step without further purification. LCMS (ESI, m/z): 314.30 [M+H]⁺.

### Intermediate A51: Synthesis of 1-(5-fluoro-1-methyl-6-(piperazin-1-yl)-1H-indazol-3-yl)dihydropyrimidine-2,4(1H,3H)-dione

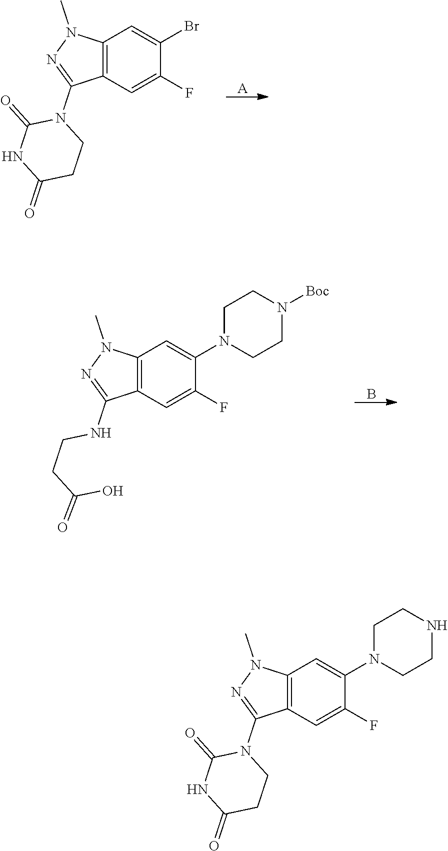

### Step A

A mixture of 1-(6-bromo-5-fluoro-1-methyl-1H-indazol-3-yl)dihydropyrimidine-2,4(1H,3H)-dione (500 mg, 1.47 mmol, 1 equiv), tert-butyl piperazine-1-carboxylate (819 mg, 4.40 mmol, 3 equiv), Pd PEPPSI IPentCl (123 mg, 0.147 mmol, 0.1 equiv) and Cs₂CO₃ (1.43 g, 4.40 mmol, 3 equiv) in 1,4-dioxane (5 mL) was stirred overnight at 100° C. under nitrogen atmosphere. The residue was purified by reversed-phase flash chromatography with the following conditions: column, C18 silica gel; mobile phase, MeCN in water (10 mmol/L NH₄HCO₃), 40% to 50% gradient in 5 min; detector, UV 254 nm to give 3-((6-(4-(tert-butoxycarbonyl)piperazin-1-yl)-5-fluoro-1-methyl-1H-indazol-3-yl)amino)propanoic acid (730 mg, 95%) as an off-white solid. LCMS (ESI, m/z): 422.20 [M+H]⁺.

### Step B

A mixture of 3-((6-(4-(tert-butoxycarbonyl)piperazin-1-yl)-5-fluoro-1-methyl-1H-indazol-3-yl)amino)propanoic acid (720 mg, 1.71 mmol, 1 equiv) and sodium cyanate (222 mg, 3.42 mmol, 2.00 equiv) in HOAc (5 mL) was stirred for overnight at 60° C. under nitrogen atmosphere. To the above mixture was added HCl (5 mL, 2 M) at 60° C. The mixture was stirred for 3 hours at 60° C. The mixture was neutralized to pH 7 with saturated aqueous NaHCO₃. The solution was concentrated and the residue was purified by reversed-phase flash chromatography with the following conditions: column, C18 silica gel; mobile phase, MeCN in water (10 mmol/L NH₄HCO₃), 5-30% gradient in 20 min and then isocratic 30% to 30% in 2 min; detector, UV 254 nm to give 1-(5-fluoro-1-methyl-6-(piperazin-1-yl)-1H-indazol-3-yl)dihydropyrimidine-2,4(1H,3H)-dione (540 mg, 91%) as an off-white solid. LCMS (ESI, m/z): 347.20 [M+H]⁺.

### Intermediate A52: Synthesis of 1-(5-fluoro-2-methoxy-4-(4-oxopiperidin-1-yl)phenyl)dihydropyrimidine-2,4(1H,3H)-dione

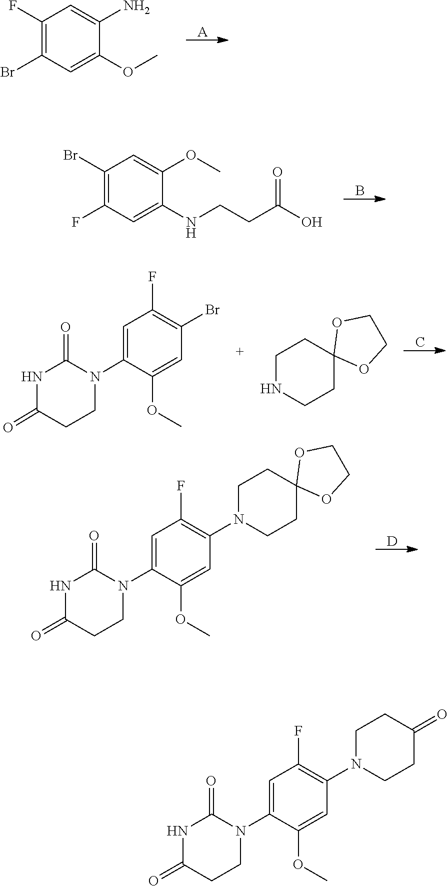

### Step A

A mixture of 4-bromo-5-fluoro-2-methoxyaniline (5 g, 22.7 mmol, 1 equiv), acrylic acid (2.46 g, 34.1 mmol, 1.5 equiv) and TBAB (0.73 g, 2.27 mmol, 0.1 equiv) in HCl (500 mL, 2M) was stirred overnight at 100° C. The mixture was neutralized to pH 8 with saturated aqueous Na₂CO₃. Then acidified to pH 5 with acetic acid. The mixture was extracted with CH₂Cl₂ (3×80 mL). The combined organic layers were washed with brine (3×50 mL), dried over anhydrous Na₂SO₄, filtered and concentrated. The residue was purified by silica gel column chromatography, eluting with PE/EA (1:9) to afford 3-((4-bromo-5-fluoro-2-methoxyphenyl)amino)propanoic acid (2.6 g, 39%) as a white solid. LCMS (ESI, m/z): 289.90 [M−H]⁻.

### Step B

A mixture of 3-((4-bromo-5-fluoro-2-methoxyphenyl)amino)propanoic acid (1.9 g, 6.51 mmol, 1 equiv) and sodium cyanate (0.85 g, 13.0 mmol, 2 equiv) in HOAc (5 mL) was stirred overnight at 60° C. Then HCl (2.5 mL, 6 M) and water (2.5 mL) were added at room temperature. The mixture was stirred for 5 hours at 60° C. then concentrated under reduced pressure. The residue was dissolved in water (5 mL). The precipitated solids were collected by filtration and washed with water (3×5 mL). The residue was purified by trituration with EtOH (10 mL). The solids were collected by filtration and washed with EtOH (3×5 mL) to afford 1-(4-bromo-5-fluoro-2-methoxyphenyl)dihydropyrimidine-2,4(1H,3H)-dione (1.2 g, 58%) as a white solid. LCMS (ESI, m/z): 316.90 [M+H]⁺.

### Step C

A mixture of 1-(4-bromo-5-fluoro-2-methoxyphenyl)dihydropyrimidine-2,4(1H,3H)-dione (0.500 g, 1.58 mmol, 1 equiv), 1,4-dioxa-8-azaspiro[4.5]decane (339 mg, 2.37 mmol, 1.5 equiv), Pd PEPPSI IPentCl (133 mg, 0.16 mmol, 0.1 equiv), and Cs₂CO₃ (240 mg, 3.15 mmol, 2 equiv) in dioxane (3 mL) was stirred overnight at 85° C. under nitrogen atmosphere. After concentration, the residue was purified by silica gel column chromatography, eluting with CH₂Cl₂/EtOH (9:1) to afford 1-(5-fluoro-2-methoxy-4-(1,4-dioxa-8-azaspiro[4.5]decan-8-yl)phenyl)dihydropyrimidine-2,4(1H,3H)-dione (0.600 g) as a white solid. The crude product was used in the next step directly without further purification LCMS (ESI, m/z): 380.10 [M+H]⁺.

### Step D

A mixture of 1-(5-fluoro-2-methoxy-4-(1,4-dioxa-8-azaspiro[4.5]decan-8-yl)phenyl)dihydropyrimidine-2,4(1H,3H)-dione (0.600 g, 1.58 mmol, 1 equiv) in conc. HCl (2 mL) and THF (4 mL) was stirred overnight. The mixture was neutralized to pH 7 with saturated aqueous NaHCO₃. The mixture was extracted with CH₂Cl₂ (3×20 mL). The combined organic layers were washed with brine (3×7 mL), dried over anhydrous Na₂SO₄, filtered and concentrated to afford 1-(5-fluoro-2-methoxy-4-(4-oxopiperidin-1-yl)phenyl)dihydropyrimidine-2,4(1H,3H)-dione (0.500 g, 94%) as a white solid. LCMS (ESI, m/z): 334.15 [M−H]⁻.

### Intermediate A53: Synthesis of 1-(7-(4-oxopiperidin-1-yl)imidazo[1,2-a]pyridin-3-yl)dihydropyrimidine-2,4(1H,3H)-dione

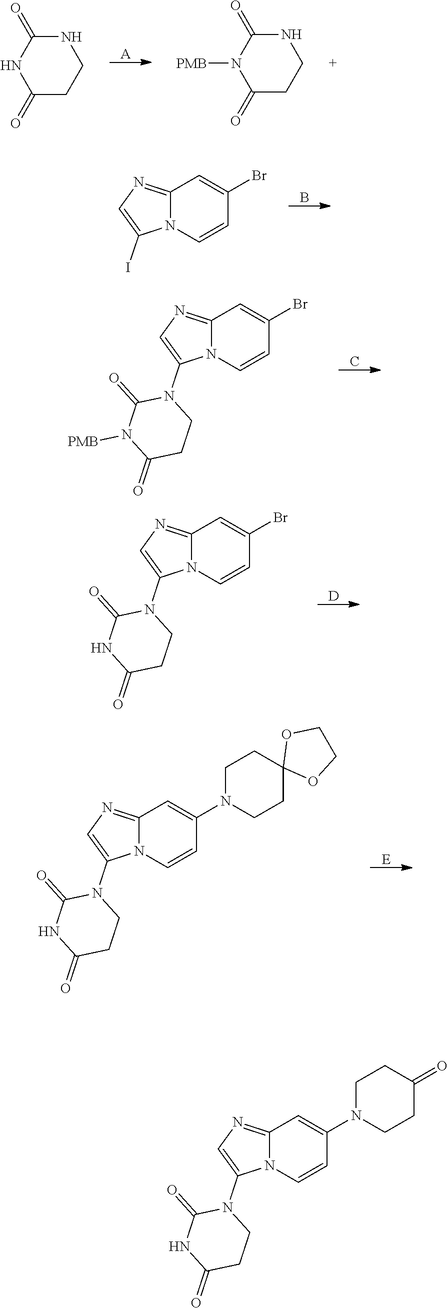

### Step A

To a solution of 4-methoxybenzyl chloride (6.86 g, 43.8 mmol, 0.5 equiv) in DMF (200 mL) was added 5,6-dihydrouracil (10 g, 87.6 mmol, 1 equiv) followed by the portion-wise addition of Cs₂CO₃ (57.1 g, 175 mmol, 2 equiv). The mixture was stirred for 3 hours. The reaction was diluted with water then extracted with EtOAc (3×500 mL). The combined organic layers were washed with brine (3×300 mL), dried over anhydrous Na₂SO₄ and filtered. The filtrate was concentrated, and the residue purified by silica gel column chromatography, eluting with PE/EA (1:19) to afford 3-(4-methoxybenzyl)dihydropyrimidine-2,4(1H,3H)-dione (4.39 g, 21%) as a white solid. LCMS (ESI, m/z): 235.20 [M+H]⁺.

### Step B

A solution of 3-[(4-methoxyphenyl)methyl]-1,3-diazinane-2,4-dione (3 g, 12.8 mmol, 1 equiv), 7-bromo-3-iodoimidazo[1,2-a]pyridine (4.96 g, 15.4 mmol, 1.2 equiv), CuI (0.49 g, 2.56 mmol, 0.2 equiv), Cs₂CO₃ (8.35 g, 25.6 mmol, 2 equiv) and (1R,2R)-1-N,2-N-dimethylcyclohexane-1,2-diamine (0.36 g, 2.56 mmol, 0.2 equiv) in DMF (30 mL) was stirred overnight at 65° C. under nitrogen atmosphere. After concentration, the residue was purified by silica gel column chromatography, eluting with PE/EtOAc (1:19). The crude product was purified by Prep-HPLC with the following conditions (Column: Welch Ultimate XB-C18 50*250 mm, 10 μm; Mobile Phase A: Water (10 mmol/L NH₄HCO₃), Mobile Phase B: ACN; Flow rate: 80 mL/min; Gradient: 35% B to 48% B in 24 min; Wavelength: 254 nm/220 nm; RT (min): 22). This afforded 1-(7-bromoimidazo[1,2-a]pyridin-3-yl)-3-(4-methoxybenzyl)dihydropyrimidine-2,4(1H,3H)-dione (1.7 g, 31%) as a white solid. LCMS (ESI, m/z): 430.95 [M+H]⁺.

### Step C

A solution of 1-(7-bromoimidazo[1,2-a]pyridin-3-yl)-3-(4-methoxybenzyl)dihydropyrimidine-2,4(1H,3H)-dione (652 mg, 1.52 mmol, 1 equiv) in TfOH (5 mL) and TFA (6 mL) was stirred overnight. The mixture was diluted with EtOAc and basified to pH 8 with saturated aqueous NaHCO₃. The mixture was filtered and the filtrate was concentrated. The residue was purified by reverse-phase flash chromatography with the following conditions: column, C18 silica gel; mobile phase, MeCN in water (10 mmol/L NH₄HCO₃), 10% to 50% gradient in 30 min; detector, UV 254 nm. This afforded 1-(7-bromoimidazo[1,2-a]pyridin-3-yl)dihydropyrimidine-2,4(1H,3H)-dione (254 mg, 54%) as a white solid. LCMS (ESI, m/z): 309.10 [M+H]⁺.

### Step D

A solution of 1-(7-bromoimidazo[1,2-a]pyridin-3-yl)dihydropyrimidine-2,4(1H,3H)-dione (270 mg, 0.873 mmol, 1 equiv), 1,4-dioxa-8-azaspiro[4.5]decane (188 mg, 1.31 mmol, 1.5 equiv), Pd PEPPSI IPentCl (73.5 mg, 0.087 mmol, 0.1 equiv) and Cs₂CO₃ (569 mg, 1.75 mmol, 2 equiv) in dioxane (6 mL) was stirred overnight at 85° C. under nitrogen atmosphere. The mixture was concentrated and the residue purified by reverse-phase flash chromatography with the following conditions: column, C18 silica gel; mobile phase, MeCN in water (10 mmol/L NH₄HCO₃), 10% to 35% gradient in 30 min; detector, UV 254 nm. This afforded in 1-(7-(1,4-dioxa-8-azaspiro[4.5]decan-8-yl)imidazo[1,2-a]pyridin-3-yl)dihydropyrimidine-2,4(1H,3H)-dione (176 mg, 54%) as a dark-green solid. LCMS (ESI, m/z): 372.05 [M+H]⁺.

### Step E

A solution of 1-(7-(1,4-dioxa-8-azaspiro[4.5]decan-8-yl)imidazo[1,2-a]pyridin-3-yl)dihydropyrimidine-2,4(1H,3H)-dione (1.53 g, 4.12 mmol, 1 equiv) in HCl in THF (25 mL, 6 M) was stirred overnight. The pH was adjusted to 8 with saturated aqueous NaHCO₃. The mixture was concentrated and the residue was purified by reversed-phase flash chromatography with the following conditions: column, C18 silica gel; mobile phase, MeCN in water (10 mmol/L NH₄HCO₃), 10% to 30% gradient in 30 min; detector, UV 254 nm. The crude compound was purified by normal phase chromatography eluting with CH₂Cl₂/MeOH (9:1) to afford 1-(7-(4-oxopiperidin-1-yl)imidazo[1,2-a]pyridin-3-yl)dihydropyrimidine-2,4(1H,3H)-dione (403 mg, 30%) as a white solid. LCMS (ESI, m/z): 328.15 [M+H]⁺. 1H NMR (400 MHz, DMSO-d6) δ 10.60 (s, 1H), 8.11 (d, J=7.6 Hz, 1H), 7.30 (s, 1H), 6.96 (dd, J=7.7, 2.5 Hz, 1H), 6.83 (d, J=2.3 Hz, 1H), 3.81-3.65 (m, 6H), 2.81 (t, J=10.0 Hz, 2H), 2.45 (t, J=6.0 Hz, 4H).

### Intermediate B1: Synthesis of 2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetic acid

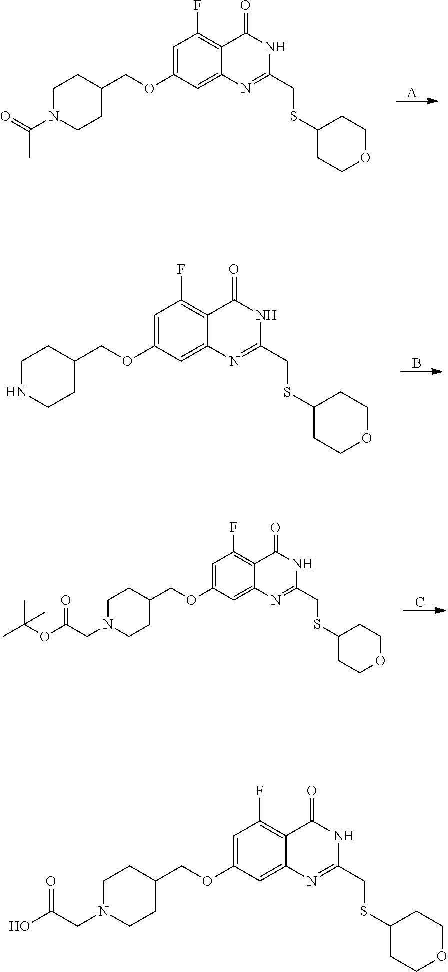

### Step A

A solution of 7-[(1-acetylpiperidin-4-yl)methoxy]-5-fluoro-2-[(oxan-4-ylsulfanyl)methyl]-3H-quinazolin-4-one (20 g, 44.5 mmol, 1.0 equiv) in aqueous NaOH (200 mL, 5 M) was stirred overnight at 80° C. The reaction was concentrated then the crude product was purified by silica gel column chromatography, eluting with DCM/MeOH (NH₃H₂O) (1:1) to afford 5-fluoro-7-(piperidin-4-ylmethoxy)-2-(((tetrahydro-2H-pyran-4-yl)thio)methyl)quinazolin-4(3H)-one (10 g, 55%) as an off-white solid. LCMS (ESI, m/z): 407.50 [M+H]⁺.

### Step B

A solution of 5-fluoro-7-(piperidin-4-ylmethoxy)-2-(((tetrahydro-2H-pyran-4-yl)thio)methyl)quinazolin-4(3H)-one (10 g, 24.5 mmol, 1.0 equiv) in MeCN (200 mL) was treated with tert-butyl 2-bromoacetate (3.83 g, 19.6 mmol, 0.8 equiv) and DIEA (9.52 g, 73.6 mmol, 3.0 equiv) and stirred for 1 hour. The resulting mixture was concentrated under reduced pressure to afford tert-butyl 2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetate (10 g) as an off-white crude solid used directly in the next step without further purification. LCMS (ESI, m/z): 521.65 [M+H]⁺.

### Step C

A solution of crude tert-butyl 2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetate (10 g, 19.1 mmol, 1.0 equiv) in TFA (200 mL) was stirred for overnight. The resulting mixture was concentrated under reduced pressure to afford 2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetic acid (8 g) as off-white crude solid used in the next step directly without further purification. LCMS (ESI, m/z): 465.54 [M+H]⁺.

Intermediate B2: Synthesis of 5-fluoro-7-((1-(prop-2-yn-1-yl)piperidin-4-yl)methoxy)-2-(((tetrahydro-2H-pyran-4-yl)thio)methyl)quinazolin-4(3H)-one

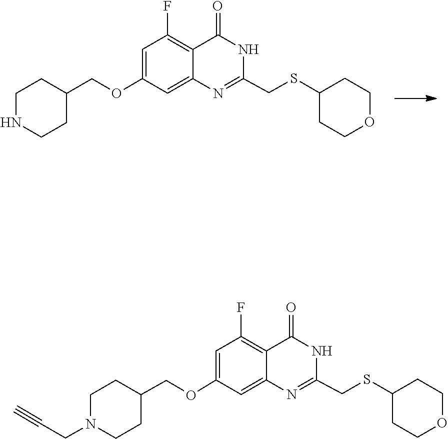 [SMILES: O=c1[nH]c(CSC2CCOCC2)nc2cc(OCC3CCNCC3)cc(F)c12 ; C#CCN1CCC(COc2cc(F)c3c(=O)[nH]c(CSC4CCOCC4)nc3c2)CC1]

To a solution of 5-fluoro-2-[(oxan-4-ylsulfanyl)methyl]-7-(piperidin-4-ylmethoxy)-3H-quinazolin-4-one (500 mg, 1.23 mmol, 1.0 equiv) in NMP (3 mL) was added DIEA (476 mg, 3.68 mmol, 3 equiv) and stirred for 15 min. Propargyl bromide (146 mg, 1.23 mmol, 1.0 equiv) was added dropwise at 0° C. and the resulting mixture was stirred for 3 hours. The reaction mixture was purified by reverse flash chromatography with the following conditions: Column, C18 silica gel; mobile phase, MeCN in water (10 mmol/L NH₄HCO₃), 10% to 50% gradient in 20 min; detector, UV 254 nm) to afford 5-fluoro-7-((1-(prop-2-yn-1-yl)piperidin-4-yl)methoxy)-2-(((tetrahydro-2H-pyran-4-yl)thio)methyl)quinazolin-4(3H)-one (155 mg, 28%) as a white solid. LCMS (ESI, m/z): 446.25 [M+H]⁺.

Intermediate B2-a was synthesized according to the procedure described for the synthesis of 5-fluoro-7-((1-(prop-2-yn-1-yl)piperidin-4-yl)methoxy)-2-(((tetrahydro-2H-pyran-4-yl)thio)methyl)quinazolin-4(3H)-one (Intermediate B2) using appropriate building blocks and modified reaction conditions (such as reagents, reagent ratio, temperature, and reaction time) and purification conditions as needed.

| Intermediate | | |
|---|---|---|
| No. | Structure | Analytical Data |
| B2-a | 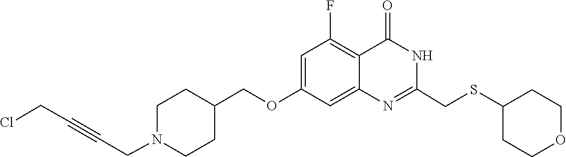 [SMILES: O=c1[nH]c(CSC2CCOCC2)nc2cc(OCC3CCN(CC#CCCl)CC3)cc(F)c12] | LCMS (ESI, m/z): 494.20 [M + H]⁺ |
| | 7-((1-(4-chlorobut-2-yn-1-yl)piperidin- | |
| | 4-yl)methoxy)-5-fluoro-2- | |
| | (((tetrahydro-2H-pyran-4- | |
| | yl)thio)methyl)quinazolin-4(3H)-one | |

### Intermediate B3: Synthesis of 7-((1-(2-chloroethyl)piperidin-4-yl)methoxy)-5-fluoro-2-(((tetrahydro-2H-pyran-4-yl)thio)methyl)quinazolin-4(3H)-one

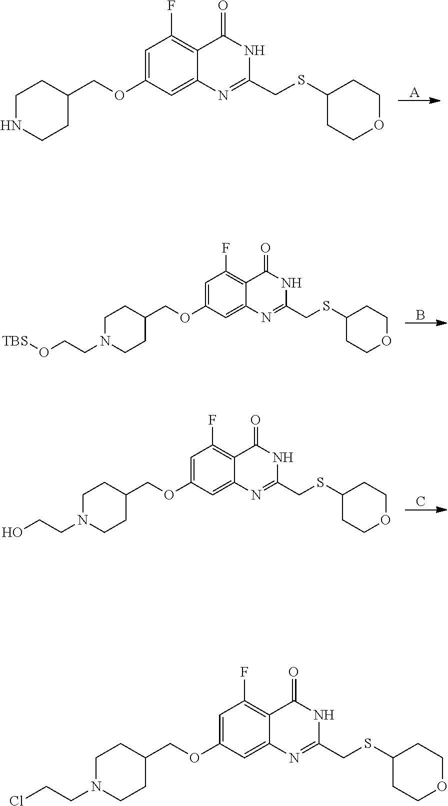

### Step A

A solution of 5-fluoro-7-(piperidin-4-ylmethoxy)-2-(((tetrahydro-2H-pyran-4-yl)thio)methyl)quinazolin-4(3H)-one (500 mg, 1.23 mmol, 1.0 equiv), DIEA (476 mg, 3.68 mmol, 3.0 equiv) and (2-bromoethoxy)(tert-butyl)dimethylsilane (294 mg, 1.23 mmol, 1.0 equiv) in NMP (5 mL) was stirred overnight at 80° C. The reaction was purified by reversed-phase flash chromatography with the following conditions: (column, C18 silica gel; mobile phase, MeCN in water (10 mmol/L NH₄HCO₃), 20% to 100% gradient in 20 min; detector, UV 254 nm) to afford 7-((1-(2-((tert-butyldimethylsilyl)oxy)ethyl)piperidin-4-yl)methoxy)-5-fluoro-2-(((tetrahydro-2H-pyran-4-yl)thio)methyl)quinazolin-4(3H)-one (205 mg, 30%) as a white solid. LCMS (ESI, m/z): 566.35 [M+H]⁺.

### Step B

A solution of 7-((1-(2-((tert-butyldimethylsilyl)oxy)ethyl)piperidin-4-yl)methoxy)-5-fluoro-2-(((tetrahydro-2H-pyran-4-yl)thio)methyl)quinazolin-4(3H)-one (205 mg, 0.36 mmol, 1.0 equiv) in HCl in 1,4-dioxane (20 mL, 4 M) was stirred for 1 hour. The resulting mixture was concentrated under reduced pressure to afford 5-fluoro-7-((1-(2-hydroxyethyl)piperidin-4-yl)methoxy)-2-(((tetrahydro-2H-pyran-4-yl)thio)methyl)quinazolin-4(3H)-one (230 mg) as a crude white solid that was used without further purification. LCMS (ESI, m/z): 452.10 [M+H]⁺.

### Step C

A solution of 5-fluoro-7-((1-(2-hydroxyethyl)piperidin-4-yl)methoxy)-2-(((tetrahydro-2H-pyran-4-yl)thio)methyl)quinazolin-4(3H)-one (230 mg, 0.51 mmol, 1.0 equiv), TEA (155 mg, 1.53 mmol, 3.0 equiv) and 3-nitrobenzenesulfonyl chloride (113 mg, 0.51 mmol, 1.0 equiv) in DCM (20 mL) was stirred for 1 hour. The resulting mixture was concentrated under reduced pressure and purified by silica gel column chromatography, eluting with MeOH/DCM (85:15) to afford 7-((1-(2-chloroethyl)piperidin-4-yl)methoxy)-5-fluoro-2-(((tetrahydro-2H-pyran-4-yl)thio)methyl)quinazolin-4(3H)-one (130 mg, 54%) as a white solid. LCMS (ESI, m/z): 470.25 [M+H]⁺.

### Intermediate B4: Synthesis of 7-((3,3-difluoropiperidin-4-yl)methoxy)-5-fluoro-2-(((tetrahydro-2H-pyran-4-yl)thio)methyl)quinazolin-4(3H)-one hydrochloride

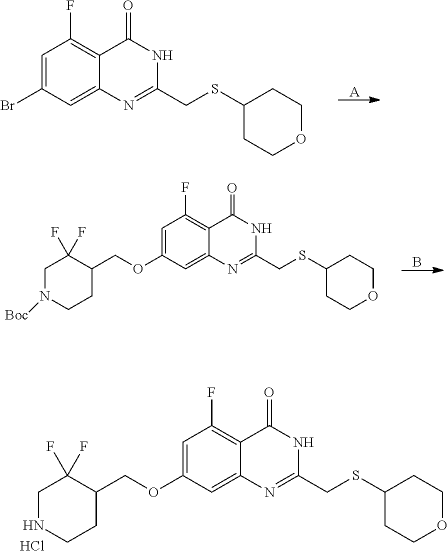

### Step A

A solution of tert-butyl 3,3-difluoro-4-(hydroxymethyl)piperidine-1-carboxylate (336 mg, 1.34 mmol, 1.0 equiv), [Pd(allyl)Cl]₂ (48.9 mg, 0.13 mmol, 0.1 equiv), RockPhos (62.7 mg, 0.13 mmol, 0.1 equiv), Cs₂CO₃ (871 mg, 2.67 mmol, 2.0 equiv) and 7-bromo-5-fluoro-2-(((tetrahydro-2H-pyran-4-yl)thio)methyl)quinazolin-4(3H)-one (499 mg, 1.34 mmol, 1.0 equiv; which can be prepared according to the processes described in U.S. Pat. No. 10,562,891) in toluene (5 mL) was stirred overnight at 80° C. under nitrogen atmosphere. The resulting mixture was concentrated under reduced pressure and the residue was purified by reversed-phase flash chromatography with the following conditions: (column, C18 silica gel; mobile phase, MeCN in Water (10 mmol/L NH₄HCO₃), 10% to 50% gradient in 10 min; detector, UV 254 nm) to tert-butyl 3,3-difluoro-4-(((5-fluoro-4-oxo-2-(((tetrahydro-2H-pyran-4-yl)thio)methyl)-3,4-dihydroquinazolin-7-yl)oxy)methyl)piperidine-1-carboxylate (289 mg, 40%) as a light yellow solid. LCMS (ESI, m/z): 544.20 [M+H]⁺.

### Step B

A solution of tert-butyl 3,3-difluoro-4-(((5-fluoro-4-oxo-2-(((tetrahydro-2H-pyran-4-yl)thio)methyl)-3,4-dihydroquinazolin-7-yl)oxy)methyl)piperidine-1-carboxylate (289 mg, 0.53 mmol, 1.0 equiv) in HCl in 1,4-dioxane (5 mL, 4 M) was stirred for 1 hour. The resulting mixture was concentrated under reduced pressure to afford 7-((3,3-difluoropiperidin-4-yl)methoxy)-5-fluoro-2-(((tetrahydro-2H-pyran-4-yl)thio)methyl)quinazolin-4(3H)-one hydrochloride (552 mg) as a crude yellow solid that was used without further purification. LCMS (ESI, m/z): 444.10 [M+H]⁺.

Intermediates B4-a and B4-b were synthesized according to the procedure described for the synthesis of 7-((3,3-difluoropiperidin-4-yl)methoxy)-5-fluoro-2-(((tetrahydro-2H-pyran-4-yl)thio)methyl)quinazolin-4(3H)-one hydrochloride (Intermediate B4) using appropriate building blocks and modified reaction conditions (such as reagents, reagent ratio, temperature, and reaction time) and purification conditions as needed.

| Example No. | Structure | Analytical Data |
|---|---|---|
| B4-a | 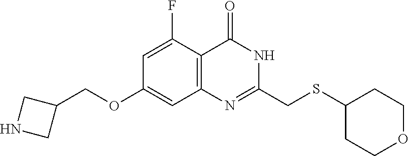 [SMILES: O=c1[nH]c(CSC2CCOCC2)nc2cc(OCC3CNC3)cc(F)c12] | LCMS (ESI, m/z): 380.30 [M + H]⁺ |
| | 7-(azetidin-3-ylmethoxy)-5-fluoro-2- | |
| | (((tetrahydro-2H-pyran-4- | |
| | yl)thio)methyl)quinazolin-4(3H)-one | |
| B4-b | 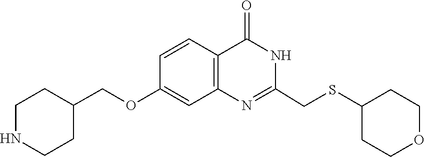 [SMILES: O=c1[nH]c(CSC2CCOCC2)nc2cc(OCC3CCNCC3)ccc12] | LCMS (ESI, m/z): 390.10 [M + H]⁺ |
| | 7-(piperidin-4-ylmethoxy)-2- | |
| | (((tetrahydro-2H-pyran-4- | |
| | yl)thio)methyl)quinazolin-4(3H)-one | |
| | HCl | |

### Intermediate B5: Synthesis of 7-bromo-5-fluoro-2-(((1-(2,2,2-trifluoroethyl)piperidin-4-yl)thio)methyl)quinazolin-4(3H)-one

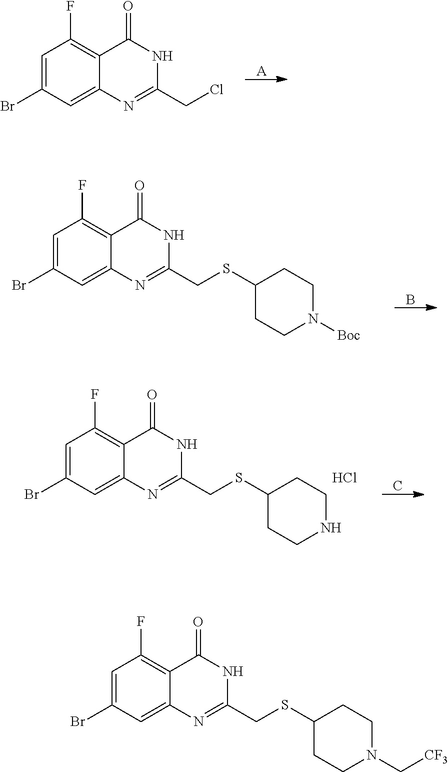

### Step A

A solution of 7-bromo-2-(chloromethyl)-5-fluoro-3H-quinazolin-4-one (1.00 g, 3.43 mmol, 1.0 equiv), tert-butyl 4-(acetylsulfanyl)piperidine-1-carboxylate (1.33 g, 5.15 mmol, 1.5 equiv) and NaOH (549 mg, 13.7 mmol, 4.0 equiv) in water (18 mL) was stirred overnight. The mixture was acidified to pH 6 with concentrated HCl. The precipitated solids were collected by filtration and washed with water (3×5 mL). The resulting solid was dried to afford tert-butyl 4-(((7-bromo-5-fluoro-4-oxo-3,4-dihydroquinazolin-2-yl)methyl)thio)piperidine-1-carboxylate (1.55 g, 96%) as a light yellow solid. LCMS (ESI, m/z): 472.06 [M+H]⁺.

### Step B

A solution of tert-butyl 4-(((7-bromo-5-fluoro-4-oxo-3,4-dihydroquinazolin-2-yl)methyl)thio)piperidine-1-carboxylate (1.5 g, 3.18 mmol, 1.0 equiv) and HCl in 1,4-dioxane (10 mL, 4 M) was stirred for 1 hour. The precipitated solids were collected by filtration and washed with 1,4-dioxane (3×5 mL). The resulting solid was dried to afford 7-bromo-5-fluoro-2-((piperidin-4-ylthio)methyl)quinazolin-4(3H)-one hydrochloride (870 mg, 67%) as an off-white solid. LCMS (ESI, m/z): 372.01 [M+H]⁺.

### Step C

To a stirred solution of NaOH (131 mg, 3.28 mmol, 2.0 equiv) and 7-bromo-5-fluoro-2-((piperidin-4-ylthio)methyl)quinazolin-4(3H)-one hydrochloride (670 mg, 1.64 mmol, 1.0 equiv) in THF (20 mL) at 0° C. was added 2,2,2-trifluoroethyl trifluoromethanesulfonate (380 mg, 1.64 mmol, 1.0 equiv) dropwise. The resulting mixture was stirred for 15 mins at 0° C. and then room temperature overnight. The resulting mixture was concentrated under reduced pressure. The residue was purified by silica gel column chromatography, eluting with EtOAc/PE (29%) to afford 7-bromo-5-fluoro-2-(((1-(2,2,2-trifluoroethyl)piperidin-4-yl)thio)methyl)quinazolin-4(3H)-one (410 mg, 55%) as a white solid. LCMS (ESI, m/z): 454.01 [M+H]⁺.

### Intermediate B6: Synthesis of 7-(chloromethyl)-5-fluoro-2-(((tetrahydro-2H-pyran-4-yl)thio)methyl)quinazolin-4(3H)-one

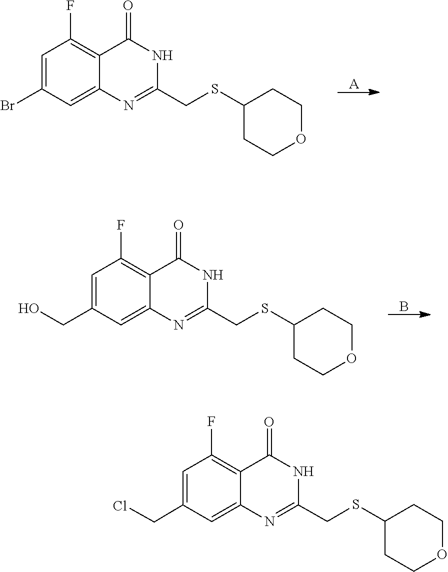

### Step A

A solution of 7-bromo-5-fluoro-2-[(oxan-4-ylsulfanyl)methyl]-3H-quinazolin-4-one (500 mg, 1.34 mmol, 1.0 equiv), (tributylstannyl)methanol (860 mg, 2.68 mmol, 2.0 equiv) and Pd(PPh₃)₄ (310 mg, 0.27 mmol, 0.2 equiv) in 1,4-dioxane (5 mL) was stirred for 1 hour at 90° C. The mixture was concentrated and the residue purified by reverse phase flash chromatography with the following conditions: column, C18 silica gel; mobile phase, MeCN in water (10 mmol/L NH₄HCO₃), 0% to 18% gradient in 20 min; detector, UV 254 nm. This afforded 5-fluoro-7-(hydroxymethyl)-2-[(oxan-4-ylsulfanyl)methyl]-3H-quinazolin-4-one (280 mg, 64%) as a white solid. LCMS (ESI, m/z): 325.10 [M+H]⁺.

### Step B

To a stirred solution of 5-fluoro-7-(hydroxymethyl)-2-[(oxan-4-ylsulfanyl)methyl]-3H-quinazolin-4-one (100 mg, 0.31 mmol, 1.0 equiv) in DCM (3 mL) was added SOCl₂ (183 mg, 1.54 mmol, 5.0 equiv) dropwise at 0° C. under nitrogen atmosphere. The resulting mixture was stirred for 1 hour at 0° C. then was concentrated under vacuum. The residue was purified by reverse phase flash chromatography with the following conditions: column, C18 silica gel; mobile phase, MeCN in water (0.1% FA), 0% to 37% gradient in 10 min; detector, UV 254 nm. This afforded 7-(chloromethyl)-5-fluoro-2-(((tetrahydro-2H-pyran-4-yl)thio)methyl)quinazolin-4(3H)-one (45 mg, 43%) as a white solid. LCMS (ESI, m/z): 343.10 [M+H]⁺.

### Intermediate B7: Synthesis of 7-((1-(2-chloroethyl)piperidin-4-yl)methoxy)-5-fluoro-2-(((tetrahydro-2H-pyran-4-yl)oxy)methyl)quinazolin-4(3H)-one

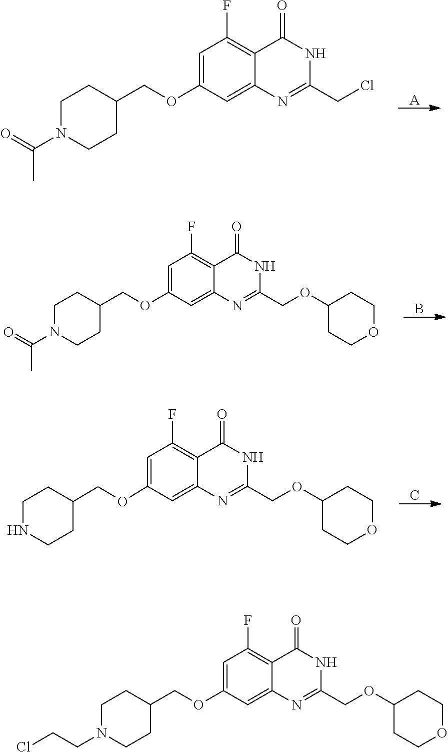

### Step A

A solution of 7-((1-acetylpiperidin-4-yl)methoxy)-2-(chloromethyl)-5-fluoroquinazolin-4(3H)-one (2.0 g, 5.44 mmol, 1.0 equiv), oxan-4-ol (0.67 g, 6.53 mmol, 1.2 equiv) and t-BuOK (1.83 g, 16.3 mmol, 3.0 equiv) in DMF (5 mL) was stirred for 2 hours. The residue was purified by reverse phase flash chromatography with the following conditions: column, C18 silica gel; mobile phase, MeCN in water (10 mmol/L NH₄HCO₃), 10% to 40% gradient in 10 min; detector, UV 254 nm. This afforded 7-((1-acetylpiperidin-4-yl)methoxy)-5-fluoro-2-(((tetrahydro-2H-pyran-4-yl)oxy)methyl)quinazolin-4(3H)-one (1.12 g, 48%) as a yellow solid. LCMS (ESI, m/z): 434.20 [M+H]⁺.

### Step B

A solution of 7-((1-acetylpiperidin-4-yl)methoxy)-5-fluoro-2-(((tetrahydro-2H-pyran-4-yl)oxy)methyl)quinazolin-4(3H)-one (1.12 g, 2.58 mmol, 1.0 equiv) and concentrated aqueous HCl (2 mL) in water (4 mL) was stirred overnight at 80° C. The resulting mixture was concentrated under reduced pressure. The residue was basified to pH 7 with saturated aqueous NaHCO₃. The residue was purified by reverse phase flash chromatography with the following conditions: column, C18 silica gel; mobile phase, MeCN in water (10 mmol/L NH₄HCO₃), 5% to 29% gradient in 10 min; detector, UV 254 nm. This afforded 5-fluoro-7-(piperidin-4-ylmethoxy)-2-(((tetrahydro-2H-pyran-4-yl)oxy)methyl)quinazolin-4(3H)-one (600 mg, 59%) as a gray solid. LCMS (ESI, m/z): 392.19 [M+H]⁺.

### Step C

A solution of 5-fluoro-7-(piperidin-4-ylmethoxy)-2-(((tetrahydro-2H-pyran-4-yl)oxy)methyl)quinazolin-4(3H)-one (350 mg, 0.82 mmol, 1.0 equiv), chloroacetaldehyde (1605 mg, 8.18 mmol, 10 equiv, 40%) and STAB (347 mg, 1.64 mmol, 2.0 equiv) in DCE (5 mL) was stirred for 1 hour. The resulting mixture was concentrated under reduced pressure. The residue was purified by silica gel column chromatography, eluting with CH₂Cl₂/MeOH (93:7) to afford 7-((1-(2-chloroethyl)piperidin-4-yl)methoxy)-5-fluoro-2-(((tetrahydro-2H-pyran-4-yl)oxy)methyl)quinazolin-4(3H)-one (170 mg, 46%) as a white solid. LCMS (ESI, m/z): 454.15 [M+H]⁺.

Intermediates B7-a through B7-c were synthesized according to the procedure described for the synthesis of 7-((1-(2-chloroethyl)piperidin-4-yl)methoxy)-5-fluoro-2-(((tetrahydro-2H-pyran-4-yl)oxy)methyl)quinazolin-4(3H)-one (Intermediate B7) using appropriate building blocks and modified reaction conditions (such as reagents, reagent ratio, temperature, and reaction time) and purification conditions as needed.

| Example No. | Structure | Analytical Data |
|---|---|---|
| B7-a | 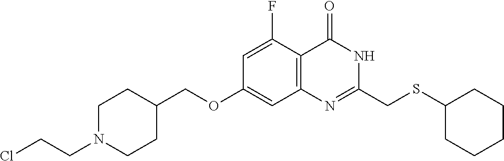 [SMILES: O=c1[nH]c(CSC2CCCCC2)nc2cc(OCC3CCN(CCCl)CC3)cc(F)c12] | LCMS (ESI, m/z): 468.03 [M + H]⁺ |
| | 7-{[1-(2-chloroethyl)piperidin-4- | |
| | yl]methoxy}-2-[(cyclohexylsulfanyl)methyl]- | |
| | 5-fluoro-3H-quinazolin-4-one | |
| B7-b | 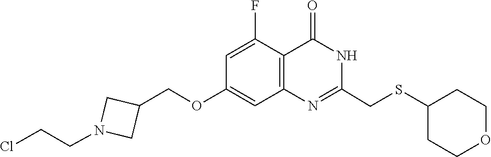 [SMILES: O=c1[nH]c(CSC2CCOCC2)nc2cc(OCC3CN(CCCl)C3)cc(F)c12] | LCMS (ESI, m/z): 442.20 [M + H]⁺ |
| | 7-((1-(2-chloroethyl)azetidin-3-yl)methoxy)- | |
| | 5-fluoro-2-(((tetrahydro-2H-pyran-4- | |
| | yl)thio)methyl)quinazolin-4(3H)-one | |
| B7-c | 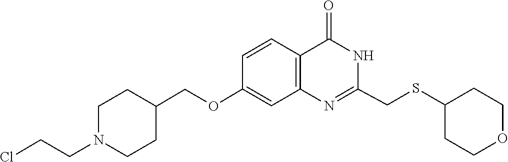 [SMILES: O=c1[nH]c(CSC2CCOCC2)nc2cc(OCC3CCN(CCCl)CC3)ccc12] | LCMS (ESI, m/z): 452.35 [M + H]⁺ |
| | 7-((1-(2-chloroethyl)piperidin-4- | |
| | yl)methoxy)-2-(((tetrahydro-2H-pyran-4- | |
| | yl)thio)methyl)quinazolin-4(3H)-one | |

### Intermediate B8: Synthesis of 5-fluoro-7-(piperidin-4-ylmethoxy)-2-(((1-(2,2,2-trifluoroethyl)piperidin-4-yl)thio)methyl)quinazolin-4(3H)-one

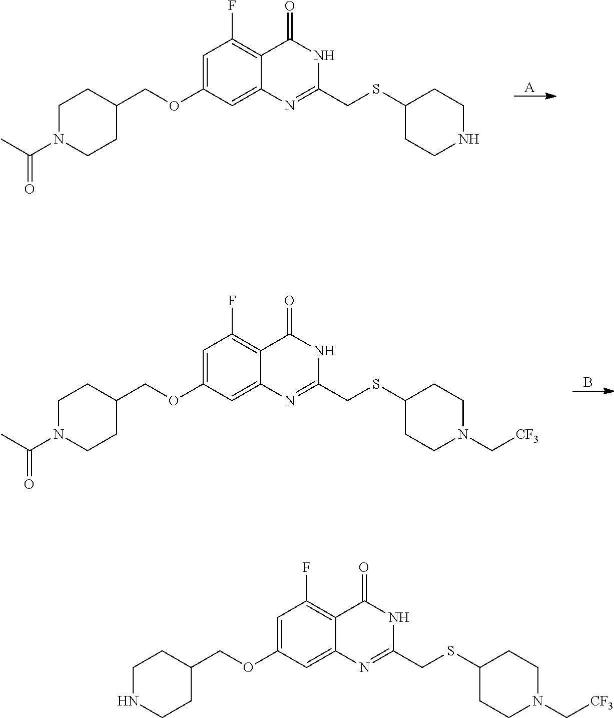

### Step A

2,2,2-trifluoroethyl trifluoromethanesulfonate (5.17 g, 22.3 mmol, 1.0 equiv) was added to a solution of 7-((1-acetylpiperidin-4-yl)methoxy)-5-fluoro-2-((piperidin-4-ylthio)methyl)quinazolin-4(3H)-one (10 g, 22 mmol, 1.0 equiv) and NaOH (1.78 g, 44.6 mmol, 2.0 equiv) in THF (60 mL) at 0° C. The resulting mixture was stirred for 2 hours. The solution was concentrated under vacuum then purified by normal phase chromatography eluting with ethyl acetate/petroleum ether (60:40) to afford 7-((1-acetylpiperidin-4-yl)methoxy)-5-fluoro-2-(((1-(2,2,2-trifluoroethyl)piperidin-4-yl)thio)methyl)quinazolin-4(3H)-one (3.3 g, 28%) as a white solid. LCMS (ESI, m/z): 531.20 [M+H]⁺.

### Step B

A solution of 7-((1-acetylpiperidin-4-yl)methoxy)-5-fluoro-2-(((1-(2,2,2-trifluoroethyl)piperidin-4-yl)thio)methyl)quinazolin-4(3H)-one (3.3 g, 6.22 mmol, 1.0 equiv) in concentrated aqueous HCl (20 mL) in water (40 mL) was stirred for 1 hour. The resulting mixture was concentrated under vacuum to afford the 5-fluoro-7-(piperidin-4-ylmethoxy)-2-(((1-(2,2,2-trifluoroethyl)piperidin-4-yl)thio)methyl)quinazolin-4(3H)-one (3.3 g) as a brown solid. The product was used in the next step without further purification. LCMS (ESI, m/z): 489.25 [M+H]⁺.

### Intermediate B9: Synthesis of 7-((1-(2-chloroethyl)piperidin-4-yl)methoxy)-2-(cyclopentylmethyl)-5-fluoroquinazolin-4(3H)-one

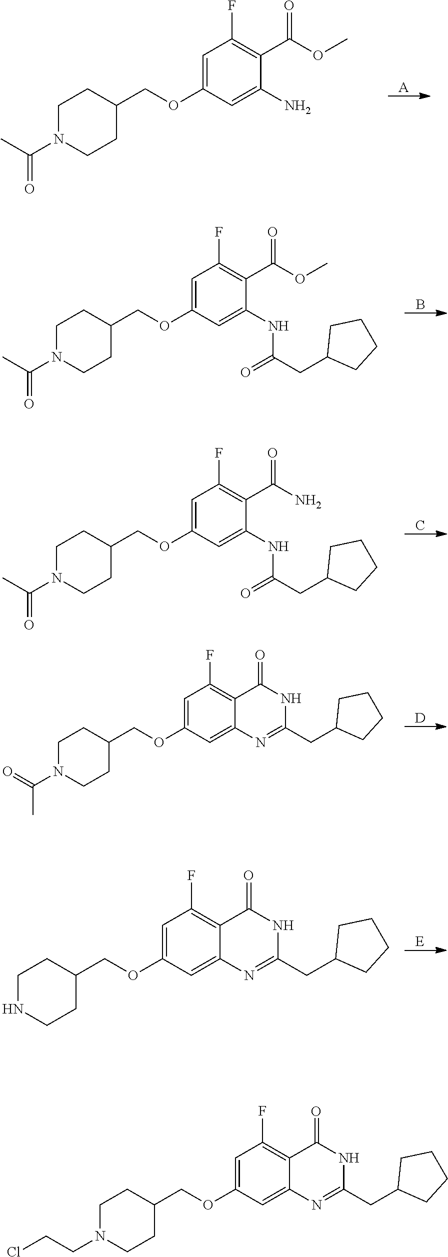

### Step A

To a stirred solution of methyl 4-[(1-acetylpiperidin-4-yl)methoxy]-2-amino-6-fluorobenzoate (5 g, 15.4 mmol, 1 equiv) and TEA (4.68 g, 46.2 mmol, 3 equiv) in DCM (50 mL) was added cyclopentylacetyl chloride (2.49 g, 17.0 mmol, 1.1 equiv) dropwise at 0° C. The resulting mixture was stirred for 2 hours at room temperature. The reaction was quenched with water and the aqueous layer was extracted with EtOAc (3×30 mL). The combined organic layers were dried over Na₂SO₄. After filtration, the filtrate was concentrated under reduced pressure to afford methyl 4-[(1-acetylpiperidin-4-yl)methoxy]-2-(2-cyclopentylacetamido)-6-fluorobenzoate (5.3 g, 79%) as a yellow solid. LCMS (ESI, m/z): 435.30 [M+H]⁺.

### Step B

A mixture of methyl 4-[(1-acetylpiperidin-4-yl)methoxy]-2-(2-cyclopentylacetamido)-6-fluorobenzoate (5.3 g, 12.2 mmol, 1 equiv) in NH₃ in MeOH (50 mL, 7 M) was stirred for 3 hours at 60° C. The resulting mixture was concentrated under reduced pressure. The residue was purified by reverse-phase flash chromatography with the following conditions: column, C18 silica gel; mobile phase, MeCN in water (0.1% FA), 0% to 100% gradient in 20 min; detector, UV 254 nm. This resulted in 4-[(1-acetylpiperidin-4-yl)methoxy]-2-(2-cyclopentylacetamido)-6-fluorobenzamide (3.4 g, 66%) as a yellow solid. LCMS (ESI, m/z): 420.20 [M+H]⁺.

### Step C

To a stirred solution of 4-[(1-acetylpiperidin-4-yl)methoxy]-2-(2-cyclopentylacetamido)-6-fluorobenzamide (3.4 g, 8.11 mmol, 1 equiv) in EtOH (30 mL) was added NaOH (0.65 g, 16.2 mmol, 2 equiv). The resulting mixture was stirred for 2 hours. The reaction was diluted with water (20 mL) and extracted with EtOAc (3×20 mL). The combined organics were concentrated under reduced pressure. This resulted in 7-[(1-acetylpiperidin-4-yl)methoxy]-2-(cyclopentylmethyl)-5-fluoro-3H-quinazolin-4-one (3.2 g, 98%) as a yellow solid. LCMS (ESI, m/z): 402.15 [M+H]⁺.

### Step D

A stirred solution of 7-[(1-acetylpiperidin-4-yl)methoxy]-2-(cyclopentylmethyl)-5-fluoro-3H-quinazolin-4-one (3.2 g, 7.97 mmol, 1 equiv) and NaOH (1.59 g, 39.9 mmol, 5 equiv) in water (30 mL) was stirred for 12 hours at 80° C. The resulting mixture was concentrated under reduced pressure. The residue was purified by reversed-phase flash chromatography with the following conditions: column, C18 silica gel; mobile phase, MeCN in water (10 mmol/L NH₄HCO₃), 0% to 70% gradient in 10 min; detector, UV 254 nm. This resulted in 2-(cyclopentylmethyl)-5-fluoro-7-(piperidin-4-ylmethoxy)-3H-quinazolin-4-one (600 mg, 21%) as a white solid. LCMS (ESI, m/z): 360.20 [M+H]⁺.

### Step E

A solution of 2-(cyclopentylmethyl)-5-fluoro-7-(piperidin-4-ylmethoxy)quinazolin-4(3H)-one (600 mg, 1.67 mmol, 1 equiv), 2-chloroacetaldehyde (655 mg, 8.35 mmol, 5 equiv) and STAB (708 mg, 3.34 mmol, 2 equiv) in DCE (20 mL) was stirred for 2 hours. The resulting mixture was concentrated under reduced pressure. The residue was purified by silica gel column chromatography, eluting with DCM/MeOH (5:1), to afford 7-((1-(2-chloroethyl)piperidin-4-yl)methoxy)-2-(cyclopentylmethyl)-5-fluoroquinazolin-4(3H)-one (500 mg, 71%) as a white solid. LCMS (ESI, m/z): 422.20 [M+H]+

### Intermediate B10: Synthesis of 5-fluoro-7-(2-(piperidin-4-yl)ethoxy)-2-(((tetrahydro-2H-pyran-4-yl)thio)methyl)quinazolin-4(3H)-one

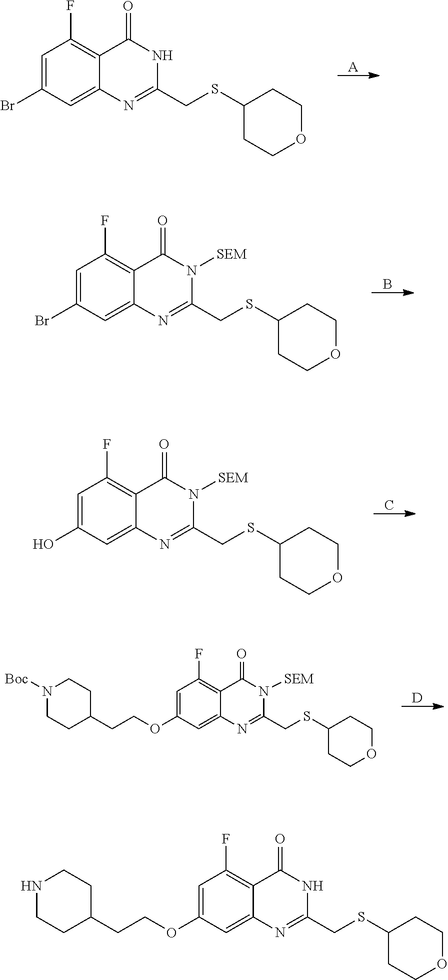

### Step A

To a stirred solution of 7-bromo-5-fluoro-2-(((tetrahydro-2H-pyran-4-yl)thio)methyl)quinazolin-4(3H)-one (4 g, 10.7 mmol, 1 equiv) and Cs₂CO₃ (10.5 g, 32.2 mmol, 3 equiv) in THF (40 mL) was added SEM-Cl (2.68 g, 16.1 mmol, 1.5 equiv) dropwise at 0° C. under nitrogen atmosphere. The resulting mixture was stirred for 1 hour at room temperature, then was concentrated under vacuum. The residue was purified by reverse-phase flash chromatography with the following conditions: column, C18 silica gel; mobile phase, MeCN in water (0.1% FA), 0% to 80% gradient in 30 min; detector, UV 254 nm to give 7-bromo-5-fluoro-2-(((tetrahydro-2H-pyran-4-yl)thio)methyl)-3-((2-(trimethylsilyl)ethoxy)methyl)quinazolin-4(3H)-one (2.5 g, 46%) as a light yellow oil. LCMS (ESI, m/z): 503.10, 505.10 [M+H]⁺.

### Step B

A solution of 7-bromo-5-fluoro-2-(((tetrahydro-2H-pyran-4-yl)thio)methyl)-3-((2-(trimethylsilyl)ethoxy)methyl)quinazolin-4(3H)-one (2.0 g, 3.97 mmol, 1 equiv), Pd₂(dba)₃ (727 mg, 0.794 mmol, 0.2 equiv), Xantphos (770 mg, 1.59 mmol, 0.4 equiv) and Cs₂CO₃ (3.88 g, 11.9 mmol, 3 equiv) in 1,4-dioxane (20 mL) and water (4 mL) was stirred for 1 hour at 90° C. under nitrogen atmosphere. The solution was concentrated and the residue was purified by reverse-phase flash chromatography with the following conditions: column, C18 silica gel; mobile phase, MeCN in water (0.1% FA), 0% to 65% gradient in 20 min; detector, UV 254 nm. This resulted in 5-fluoro-7-hydroxy-2-(((tetrahydro-2H-pyran-4-yl)thio)methyl)-3-((2-(trimethylsilyl)ethoxy)methyl)quinazolin-4(3H)-one (490 mg, 28%) as a yellow solid. LCMS (ESI, m/z): 441.30 [M+H]⁺.

### Step C

A solution of 5-fluoro-7-hydroxy-2-(((tetrahydro-2H-pyran-4-yl)thio)methyl)-3-((2-(trimethylsilyl)ethoxy)methyl)quinazolin-4(3H)-one (600 mg, 1.36 mmol, 1 equiv), tert-butyl 4-(2-hydroxyethyl)piperidine-1-carboxylate (659 mg, 2.87 mmol, 2.11 equiv), DEAD (363 mg, 2.08 mmol, 1.53 equiv) and PPh₃ (540 mg, 2.06 mmol, 1.51 equiv) in THF (12 mL) was stirred for 1 hour under nitrogen atmosphere. The resulting mixture was concentrated under vacuum. The residue was purified by silica gel column chromatography, eluting with PE/EtOAc (3:1) to afford tert-butyl 4-(2-((5-fluoro-4-oxo-2-(((tetrahydro-2H-pyran-4-yl)thio)methyl)-3-((2-(trimethylsilyl)ethoxy)methyl)-3,4-dihydroquinazolin-7-yl)oxy)ethyl)piperidine-1-carboxylate (1 g, 90%) as a yellow solid. LCMS (ESI, m/z): 652.45 [M+H]⁺.

### Step D

A solution of tert-butyl 4-(2-((5-fluoro-4-oxo-2-(((tetrahydro-2H-pyran-4-yl)thio)methyl)-3-((2-(trimethylsilyl)ethoxy)methyl)-3,4-dihydroquinazolin-7-yl)oxy)ethyl)piperidine-1-carboxylate (1 g, 1.53 mmol, 1 equiv) in HCl in 1,4-dioxane (8 mL, 4 M) and MeOH (8 mL) was stirred overnight. The resulting mixture was concentrated under vacuum to afford 5-fluoro-7-(2-(piperidin-4-yl)ethoxy)-2-(((tetrahydro-2H-pyran-4-yl)thio)methyl)quinazolin-4(3H)-one (850 mg, 99%) as a white solid. LCMS (ESI, m/z): 422.30 [M+H]⁺.

Intermediate B11: Synthesis of 7-((1-(2-azaspiro[3.5]nonan-7-yl)piperidin-4-yl)methoxy)-5-fluoro-2-(((tetrahydro-2H-pyran-4-yl)thio)methyl)quinazolin-4(3H)-one

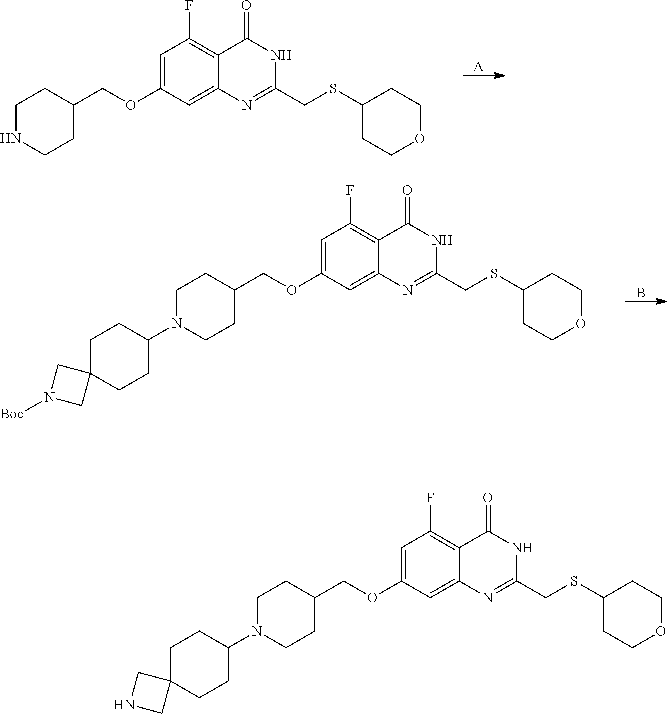

### Step A

A solution of 5-fluoro-2-[(oxan-4-ylsulfanyl)methyl]-7-(piperidin-4-ylmethoxy)-3H-quinazolin-4-one (2.2 g, 5.4 mmol, 1 equiv) in DCE (20 mL) was treated with tert-butyl 7-oxo-2-azaspiro[3.5]nonane-2-carboxylate (3.88 g, 16.2 mmol, 3 equiv) for 2 hours followed by the addition of STAB (5.72 g, 27.0 mmol, 5 equiv) portion-wise. The resulting mixture was stirred for 2 hours at 60° C. The resulting mixture was concentrated under reduced pressure. The residue was purified by silica gel column chromatography, eluting with DCM/MeOH (3:22) to afford tert-butyl 7-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-azaspiro[3.5]nonane-2-carboxylate (1.9 g, 56%) as a yellow solid. LCMS (ESI, m/z): 631.35 [M+H]⁺.

### Step B

A solution of tert-butyl 7-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-azaspiro[3.5]nonane-2-carboxylate (1.9 g, 3.01 mmol, 1 equiv) in TFA (5 mL) and DCM (20 mL) was stirred for 1 hour. The resulting mixture was concentrated under reduced pressure to afford crude 7-((1-(2-azaspiro[3.5]nonan-7-yl)piperidin-4-yl)methoxy)-5-fluoro-2-(((tetrahydro-2H-pyran-4-yl)thio)methyl)quinazolin-4(3H)-one (4.8 g) as a brown oil. The crude product was used in the next step without further purification. LCMS (ESI, m/z): 531.30 [M+H]⁺.

Intermediate B11-a was synthesized according to the procedure described for the synthesis of 7-((1-(2-azaspiro[3.5]nonan-7-yl)piperidin-4-yl)methoxy)-5-fluoro-2-(((tetrahydro-2H-pyran-4-yl)thio)methyl)quinazolin-4(3H)-one (Intermediate B11) using appropriate building blocks and modified reaction conditions (such as reagents, reagent ratio, temperature, and reaction time) and purification conditions as needed.

| Example No. | Structure | Analytical Data |
|---|---|---|
| B11-a | 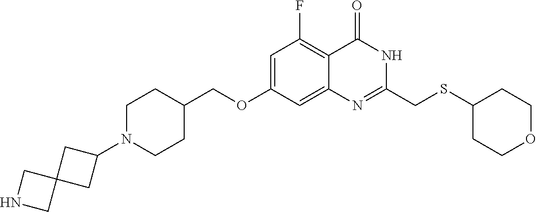 [SMILES: O=c1[nH]c(CSC2CCOCC2)nc2cc(OCC3CCN(C4CC5(CNC5)C4)CC3)cc(F)c12] | LCMS (ESI, m/z): 503.65 [M + H]+ |
| | 7-[(1-{2-azaspiro[3.3]heptan-6- | |
| | yl}piperidin-4-yl)methoxy]-5-fluoro-2- | |
| | [(oxan-4-ylsulfanyl)methyl]-3H-quinazolin- | |
| | 4-one | |

Intermediates B11-b-B11-f were synthesized according to the procedure described for the synthesis of 7-((1-(2-azaspiro[3.5]nonan-7-yl)piperidin-4-yl)methoxy)-5-fluoro-2-(((tetrahydro-2H-pyran-4-yl)thio)methyl)quinazolin-4(3H)-one (Intermediate Bit) using appropriate building blocks and modified reaction conditions (such as reagents (e.g., HCl in place of TFA), reagent ratio, temperature, and reaction time) and purification conditions as needed.

| Example No. | Structure | Analytical Data |
|---|---|---|
| B11-b | 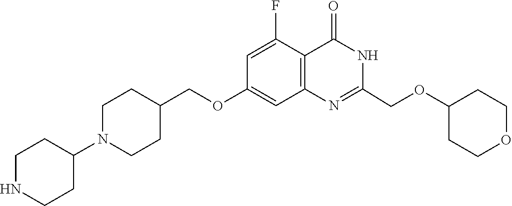 [SMILES: O=c1[nH]c(COC2CCOCC2)nc2cc(OCC3CCN(C4CCNCC4)CC3)cc(F)c12] | LCMS (ESI, m/z): 475.20 [M + H]⁺ |
| | 7-([1,4′-bipiperidin]-4-ylmethoxy)-5-fluoro-2- | |
| | (((tetrahydro-2H-pyran-4- | |
| | yl)oxy)methyl)quinazolin-4(3H)-one | |
| B11-c | 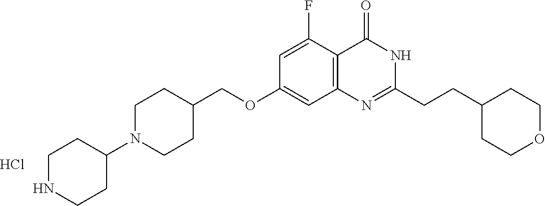 [SMILES: Cl ; O=c1[nH]c(CCC2CCOCC2)nc2cc(OCC3CCN(C4CCNCC4)CC3)cc(F)c12] | LCMS (ESI, m/z): 473.10 [M + H]⁺ |
| | 7-([1,4′-bipiperidin]-4-ylmethoxy)-5-fluoro-2-(2- | |
| | (tetrahydro-2H-pyran-4-yl)ethyl)quinazolin- | |
| | 4(3H)-one hydrogen chloride | |
| B11-d | 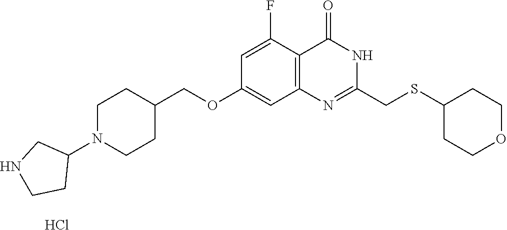 [SMILES: O=c1[nH]c(CSC2CCOCC2)nc2cc(OCC3CCN(C4CCNC4)CC3)cc(F)c12 ; Cl] | LCMS (ESI, m/z): 513.15 [M + H]⁺ |
| | 5-fluoro-7-((1-(pyrrolidin-3-yl)piperidin-4- | |
| | yl)methoxy)-2-(((tetrahydro-2H-pyran-4- | |
| | yl)thio)methyl)quinazolin-4(3H)-one, hydrogen | |
| | chloride | |
| B11-e | 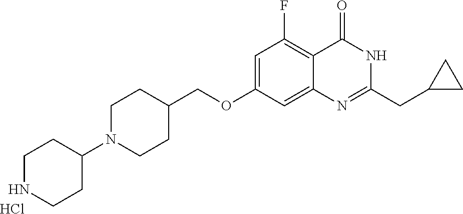 [SMILES: O=c1[nH]c(CC2CC2)nc2cc(OCC3CCN(C4CCNCC4)CC3)cc(F)c12 ; Cl] | LCMS (ESI, m/z): 415.25 [M + H]⁺ |
| | 7-([1,4′-bipiperidin]-4-ylmethoxy)-2- | |
| | (cyclopropylmethyl)-5-fluoroquinazolin-4(3H)- | |
| | one hydrochloride | |
| B11-f | 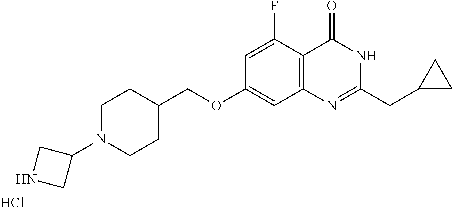 [SMILES: O=c1[nH]c(CC2CC2)nc2cc(OCC3CCN(C4CNC4)CC3)cc(F)c12 ; Cl] | LCMS (ESI, m/z): 387.25 [M + H]⁺ |
| | 7-((1-(azetidin-3-yl)piperidin-4-yl)methoxy)-2- | |
| | (cyclopropylmethyl)-5-fluoroquinazolin-4(3H)- | |
| | one hydrochloride | |

### Intermediate B12: Synthesis of 7-((1-(2-chloroethyl)piperidin-4-yl)methoxy)-2-(cyclopropylmethyl)-5-fluoroquinazolin-4(3H)-one

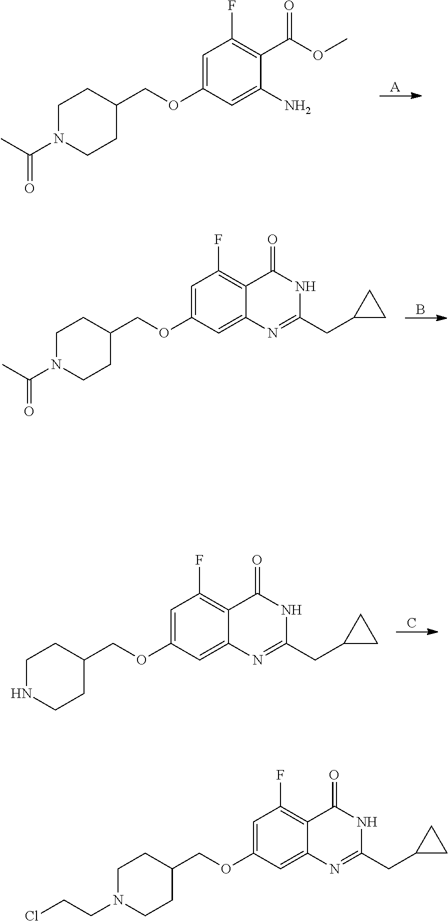

### Step A

A solution of methyl 4-((1-acetylpiperidin-4-yl)methoxy)-2-amino-6-fluorobenzoate (1 g, 3.08 mmol, 1 equiv) and 2-cyclopropylacetonitrile (1500 mg, 18.5 mmol, 6 equiv) in HCl in 1,4-dioxane (10 mL, 4 M) was stirred for 6 hours at 90° C. The resulting mixture was concentrated under reduced pressure. The residue was purified by silica gel column chromatography, eluting with DCM/MeOH (92:8) to afford 7-((1-acetylpiperidin-4-yl)methoxy)-2-(cyclopropylmethyl)-5-fluoroquinazolin-4(3H)-one (1 g, 87%) as a yellow solid. LCMS (ESI, m/z): 410.50 [M+H]⁺.

### Step B

A solution of 7-((1-acetylpiperidin-4-yl)methoxy)-2-(cyclopropylmethyl)-5-fluoroquinazolin-4(3H)-one (1 g, 2.68 mmol, 1 equiv) in aqueous HCl (14 mL, 4 M) was stirred for 6 hours at 80° C. The mixture was neutralized to pH 8 with K₂CO₃. The precipitated solids were collected by filtration and washed with water (5×10 mL). The resulting solid was dried in an oven. The residue was then purified by silica gel column chromatography, eluting with DCM/MeOH (85:15) to afford 2-(cyclopropylmethyl)-5-fluoro-7-(piperidin-4-ylmethoxy)quinazolin-4(3H)-one (602 mg, 69%) as a white solid. LCMS (ESI, m/z): 332.25 [M+H]⁺.

### Step C

A solution of 2-(cyclopropylmethyl)-5-fluoro-7-(piperidin-4-ylmethoxy)quinazolin-4(3H)-one (592 mg, 1.79 mmol, 1 equiv) and chloroacetaldehyde (982 mg, 12.5 mmol, 7 equiv) in DCE (6 mL) was stirred for 1 hour. The solution was concentrated and the residue was purified by silica gel column chromatography, eluting with DCM/MeOH (92:8) to afford 7-((1-(2-chloroethyl)piperidin-4-yl)methoxy)-2-(cyclopropylmethyl)-5-fluoroquinazolin-4(3H)-one (526 mg, 75%) as an orange oil. LCMS (ESI, m/z): 394.25 [M+H]⁺.

Intermediates B12-a and B12-b was synthesized according to the procedure described for the synthesis of 7-((1-(2-chloroethyl)piperidin-4-yl)methoxy)-2-(cyclopropylmethyl)-5-fluoroquinazolin-4(3H)-one (Intermediate B12) using appropriate building blocks and modified reaction conditions (such as reagents, reagent ratio, temperature, and reaction time) and purification conditions as needed.

| Example No. | Structure | Analytical Data |
|---|---|---|
| B12-a | 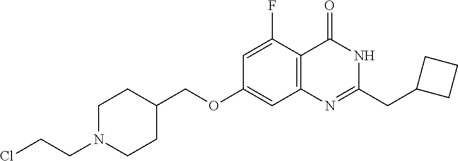 [SMILES: O=c1[nH]c(CC2CCC2)nc2cc(OCC3CCN(CCCl)CC3)cc(F)c12] | LCMS (ESI, m/z): 408.25 [M + H]⁺ |
| | 7-((1-(2-chloroethyl)piperidin-4- | |
| | yl)methoxy)-2-(cyclobutylmethyl)-5- | |
| | fluoroquinazolin-4(3H)-one | |
| B12-b | 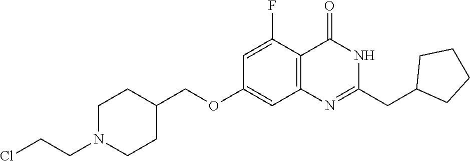 [SMILES: O=c1[nH]c(CC2CCCC2)nc2cc(OCC3CCN(CCCl)CC3)cc(F)c12] | LCMS (ESI, m/z): 422.35 [M + H]⁺ |
| | 7-((4-(2- | |
| | chloroethyl)cyclohexyl)methoxy)-2- | |
| | (cyclopentylmethyl)-5- | |
| | fluoroquinazolin-4(3H)-one | |

### Intermediate B13: Synthesis of 5-fluoro-7-((1-(piperidine-4-carbonyl)piperidin-4-yl)methoxy)-2-(((tetrahydro-2H-pyran-4-yl)thio)methyl)quinazolin-4(3H)-one hydrochloride

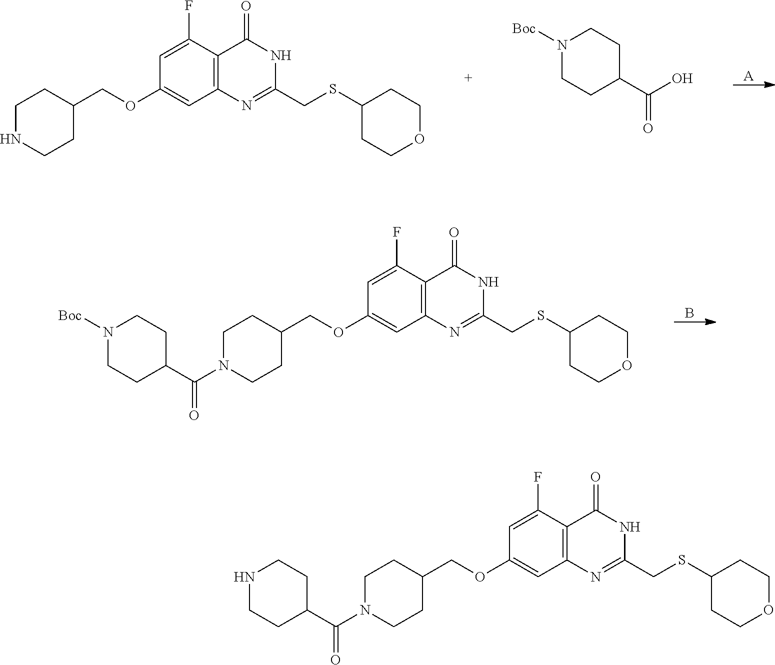

### Step A

A mixture of 5-fluoro-7-(piperidin-4-ylmethoxy)-2-(((tetrahydro-2H-pyran-4-yl)thio)methyl)quinazolin-4(3H)-one (8.0 g, 20 mmol, 1 equiv), 1-(tert-butoxycarbonyl)piperidine-4-carboxylic acid (4.95 g, 21.6 mmol, 1.1 equiv), HATU (8.96 g, 23.6 mmol, 1.2 equiv) and DIEA (7.61 g, 58.9 mmol, 3 equiv) in DMF (30 mL) was stirred for 4 hours. The solution was diluted with water and extracted with EtOAc (3×30 mL). The combined organic layers were concentrated under reduced pressure. The residue was purified by silica gel column chromatography, eluting with DCM/MeOH (10:1) to afford tert-butyl 4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperidine-1-carboxylate (9.0 g, 74%) as a yellow solid. LCMS (ESI, m/z): 619.05 [M+H]⁺.

### Step B

A mixture of tert-butyl 4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperidine-1-carboxylate (9.0 g, 15 mmol, 1 equiv) in HCl in 1,4-dioxane (50 mL, 4 M) was stirred for 1 hour. The mixture was concentrated to dryness to afford 5-fluoro-7-((1-(piperidine-4-carbonyl)piperidin-4-yl)methoxy)-2-(((tetrahydro-2H-pyran-4-yl)thio)methyl)quinazolin-4(3H)-one hydrochloride (6.7 g, 89%) as a white solid. LCMS (ESI, m/z): 519.10 [M+H]⁺.

### Intermediate B14: Synthesis of 5-fluoro-7-(piperidin-4-ylethynyl)-2-(((tetrahydro-2H-pyran-4-yl)thio)methyl)quinazolin-4(3H)-one hydrochloride

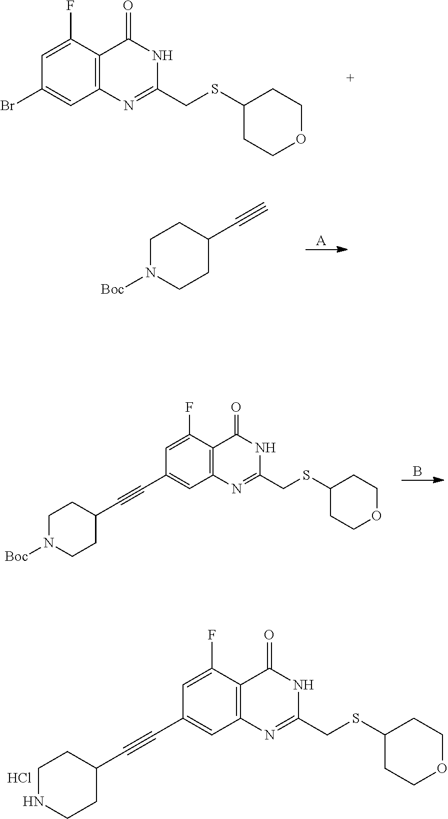

### Step A

A solution of 7-bromo-5-fluoro-2-(((tetrahydro-2H-pyran-4-yl)thio)methyl)quinazolin-4(3H)-one (5.0 g, 13 mmol, 1 equiv), tert-butyl 4-ethynylpiperidine-1-carboxylate (4.21 g, 20.1 mmol, 1.5 equiv), Pd(PPh₃)₂Cl₂ (0.94 g, 1.34 mmol, 0.1 equiv) and CuI (0.26 g, 1.34 mmol, 0.1 equiv) in DMSO (40 mL) and TEA (10 mL) was stirred for 2 hours at 80° C. under nitrogen atmosphere. The residue was dissolved in water (20 mL). The aqueous layer was extracted with EtOAc (3×20 mL) and the mixture was washed with 3×20 mL of brine. The organic layers was concentrated under reduced pressure. The residue was purified by silica gel column chromatography, eluting with DCM/MeOH (93:7) to afford tert-butyl 4-((5-fluoro-4-oxo-2-(((tetrahydro-2H-pyran-4-yl)thio)methyl)-3,4-dihydroquinazolin-7-yl)ethynyl)piperidine-1-carboxylate (5.9 g, 88%) as a red solid. LCMS (ESI, m/z): 502.21 [M+H]⁺.

### Step B

A solution of tert-butyl 4-((5-fluoro-4-oxo-2-(((tetrahydro-2H-pyran-4-yl)thio)methyl)-3,4-dihydroquinazolin-7-yl)ethynyl)piperidine-1-carboxylate (3.0 g, 6.0 mmol, 1 equiv) in HCl in 1,4-dioxane (20 mL, 4 M) was stirred for 30 min. The mixture was concentrated to dryness to give 5-fluoro-7-(piperidin-4-ylethynyl)-2-(((tetrahydro-2H-pyran-4-yl)thio)methyl)quinazolin-4(3H)-one hydrochloride (3.6 g) as a red solid. The crude product was used in the next step directly without further purification. LCMS (ESI, m/z): 402.16 [M+H]⁺.

### Intermediate B15: Synthesis of 5-fluoro-7-((1-(2-(piperazin-1-yl)ethyl)piperidin-4-yl)methoxy)-2-(((tetrahydro-2H-pyran-4-yl)thio)methyl)quinazolin-4(3H)-one hydrochloride

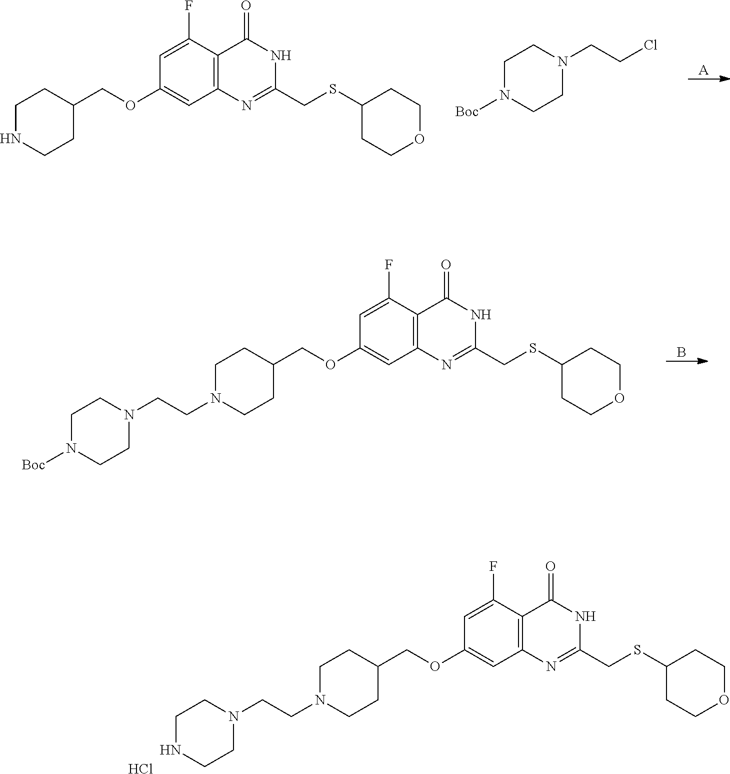

### Step A

A solution of 5-fluoro-7-(piperidin-4-ylmethoxy)-2-(((tetrahydro-2H-pyran-4-yl)thio)methyl)quinazolin-4(3H)-one (1.70 kg, 4172 mmol, 1 equiv), tert-butyl 4-(2-chloroethyl)piperazine-1-carboxylate (1.09 kg, 4380 mmol, 1.05 equiv) and NaHCO₃ (0.70 kg, 8344 mmol, 2 equiv) in water (3 L) and EtOH (15 L) was stirred overnight at 80° C. The mixture was concentrated and the residue purified by silica gel column chromatography, eluting with DCM/MeOH (25:1) to afford tert-butyl 4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazine-1-carboxylate (1.6 kg, 62%) as a yellow solid. LCMS (ESI, m/z): 620.15 [M+H]⁺.

### Step B

A solution of tert-butyl 4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazine-1-carboxylate (1.6 kg, 2581 mmol, 1 equiv) in HCl in 1,4-dioxane (10 L, 4 M) was stirred for 5 hours. The mixture was diluted with PE (2 L) and the precipitated solids collected by filtration and washed with PE (3×400 mL). The collected solid was dried in an oven to afford 5-fluoro-7-((1-(2-(piperazin-1-yl)ethyl)piperidin-4-yl)methoxy)-2-(((tetrahydro-2H-pyran-4-yl)thio)methyl)quinazolin-4(3H)-one hydrochloride (1.70 kg) as a white crude solid. The crude material was used in the next step directly without further purification. LCMS (ESI, m/z):520.35 [M+H]⁺.

### Example 1: Synthesis of N-(6-((2-(2,6-dioxopiperidin-3-yl)-1,3-dioxoisoindolin-4-yl)amino)hexyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide

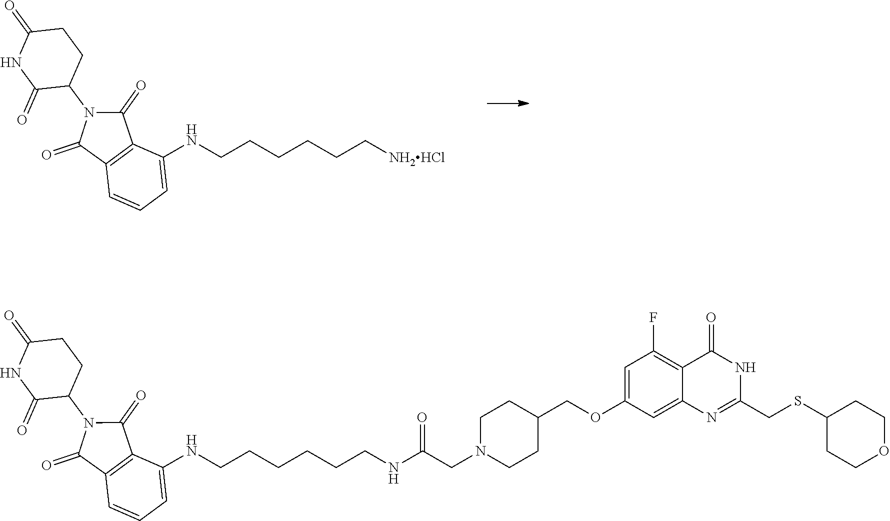

A solution of 2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetic acid (350 mg, 0.75 mmol, 1.0 equiv) and 4-((6-aminohexyl)amino)-2-(2,6-dioxopiperidin-3-yl)isoindoline-1,3-dione hydrochloride (307 mg, 0.75 mmol, 1.0 equiv) DIEA (292 mg, 2.26 mmol, 3.0 equiv) and HATU (343 mg, 0.902 mmol, 1.2 equiv) in DMF (5 mL) was stirred for 2 hours. The reaction was purified by reverse flash chromatography with the following conditions: (column, C18 silica gel; mobile phase, MeCN in water (10 mmol/L NH₄HCO₃), 5% to 52% gradient in 15 min; detector, UV 254 nm) and further purified by Prep-HPLC with the following condition (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 33% B to 55% B in 9 min, 55% B; Wave Length: 254/220 nm; RT (min): 9) to afford N-(6-((2-(2,6-dioxopiperidin-3-yl)-1,3-dioxoisoindolin-4-yl)amino)hexyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide (113 mg, 18%) as a light yellow solid. LCMS (ESI, m/z): 820.40 [M+H]⁺. ¹H NMR (400 MHz, DMSO-d₆) δ 12.16 (s, 1H), 11.09 (s, 1H), 7.64 (t, J=6.0 Hz, 1H), 7.57 (t, J=8.0 Hz, 1H), 7.07 (d, J=8.4 Hz, 1H), 7.00 (d, J=6.8 Hz, 1H), 6.87 (s, 1H), 6.84 (s, 1H), 6.52 (t, J=5.2 Hz, 1H), 5.04 (dd, J=12.8, 5.2 Hz, 1H), 3.96 (d, J=5.2 Hz, 2H), 3.81 (d, J=11.2 Hz, 2H), 3.61 (s, 2H), 3.37-3.32 (m, 1H), 3.30-3.23 (m, 3H), 3.13-3.00 (m, 3H), 2.94-2.75 (m, 5H), 2.62-2.51 (m, 2H), 2.08-1.94 (M, 3H), 1.88 (d, J=13.2 Hz, 2H), 1.72 (d, J=11.3 Hz, 3H), 1.61-1.52 (m, 2H), 1.50-1.37 (m, 4H), 1.36-1.23 (m, 6H).

Examples 2-20 were synthesized according to the procedure described for the synthesis of N-(6-((2-(2,6-dioxopiperidin-3-yl)-1,3-dioxoisoindolin-4-yl)amino)hexyl)-2-(4-(((5-fluoro-4-oxo-2-(((tetrahydro-2H-pyran-4-yl)thio)methyl)-3,4-dihydroquinazolin-7-yl)oxy)methyl)piperidin-1l-yl)acetamide (Example 1) using appropriate building blocks and modified reaction conditions (such as reagents, reagent ratio, temperature, and reaction time) and purification conditions as needed.

| Ex- | |
|---|---|
| am- | |
| ple | Structure |
| 2 | 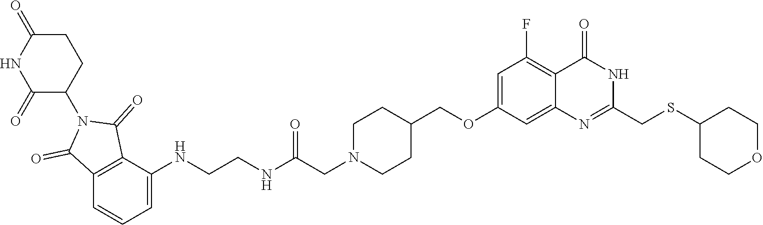 [SMILES: O=C(CN1CCC(COc2cc(F)c3c(=O)[nH]c(CSC4CCOCC4)nc3c2)CC1)NCCNc1cccc2c1C(=O)N(C1CCC(=O)NC1=O)C2=O] |
| | N-(2-((2-(2,6-dioxopiperidin-3-yl)-1,3-dioxoisoindolin-4- |
| | yl)amino)ethyl)-2-(4-(((5-fluoro-4-oxo-2-(((tetrahydro-2H-pyran- |
| | 4-yl)thio)methyl)-3,4-dihydroquinazolin-7- |
| | yl)oxy)methyl)piperidin-1-yl)acetamide |
| 3 | 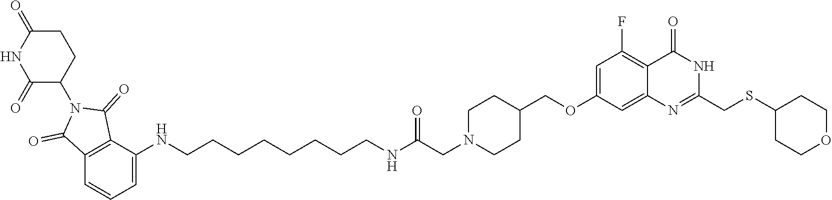 [SMILES: O=C(CN1CCC(COc2cc(F)c3c(=O)[nH]c(CSC4CCOCC4)nc3c2)CC1)NCCCCCCCCNc1cccc2c1C(=O)N(C1CCC(=O)NC1=O)C2=O] |
| | N-(8-((2-(2,6-dioxopiperidin-3-yl)-1,3-dioxoisoindolin-4- |
| | yl)amino)octyl)-2-(4-(((5-fluoro-4-oxo-2-(((tetrahydro-2H-pyran- |
| | 4-yl)thio)methyl)-3,4-dihydroquinazolin-7- |
| | yl)oxy)methyl)piperidin-1-yl)acetamide |
| 4 | 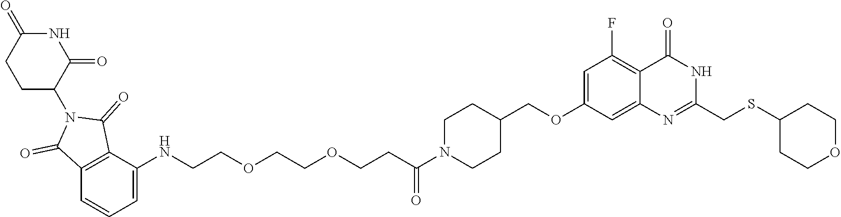 [SMILES: O=C1CCC(N2C(=O)c3cccc(NCCOCCOCCC(=O)N4CCC(COc5cc(F)c6c(=O)[nH]c(CSC7CCOCC7)nc6c5)CC4)c3C2=O)C(=O)N1] |
| | 2-(2,6-dioxopiperidin-3-yl)-4-((2-(2-(3-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-3- |
| | oxopropoxy)ethoxy)ethyl)amino)isoindoline-1,3-dione |
| 5 | 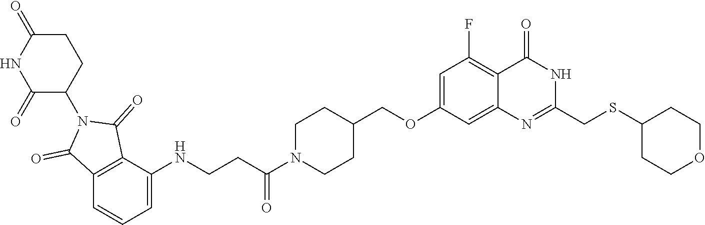 [SMILES: O=C1CCC(N2C(=O)c3cccc(NCCC(=O)N4CCC(COc5cc(F)c6c(=O)[nH]c(CSC7CCOCC7)nc6c5)CC4)c3C2=O)C(=O)N1] |
| | 2-(2,6-dioxopiperidin-3-yl)-4-((3-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-3-oxopropyl)amino)isoindoline-1,3- |
| | dione |
| 6 | 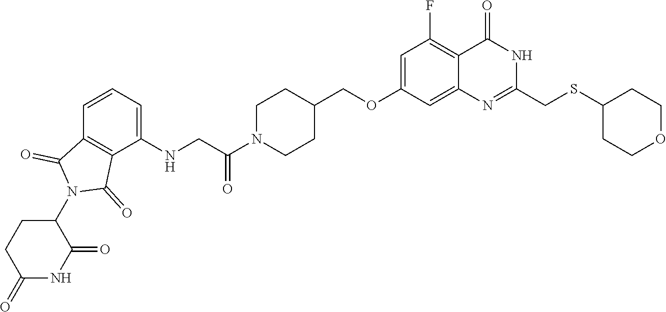 [SMILES: O=C1CCC(N2C(=O)c3cccc(NCC(=O)N4CCC(COc5cc(F)c6c(=O)[nH]c(CSC7CCOCC7)nc6c5)CC4)c3C2=O)C(=O)N1] |
| | 2-(2,6-dioxopiperidin-3-yl)-4-((2-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-2-oxoethyl)amino)isoindoline-1,3- |
| | dione |
| 7 | 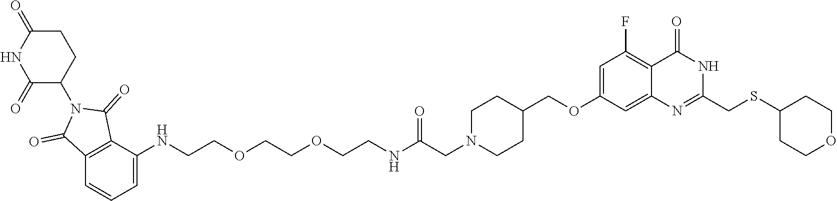 [SMILES: O=C(CN1CCC(COc2cc(F)c3c(=O)[nH]c(CSC4CCOCC4)nc3c2)CC1)NCCOCCOCCNc1cccc2c1C(=O)N(C1CCC(=O)NC1=O)C2=O] |
| | N-(2-(2-(2-((2-(2,6-dioxopiperidin-3-yl)-1,3-dioxoisoindolin-4- |
| | yl)amino)ethoxy)ethoxy)ethyl)-2-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)acetamide |
| 8 | 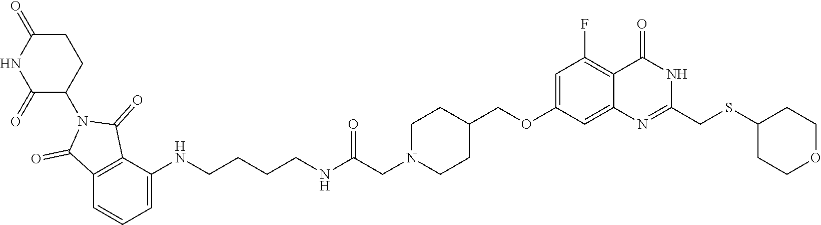 [SMILES: O=C(CN1CCC(COc2cc(F)c3c(=O)[nH]c(CSC4CCOCC4)nc3c2)CC1)NCCCCNc1cccc2c1C(=O)N(C1CCC(=O)NC1=O)C2=O] |
| | N-(4-((2-(2,6-dioxopiperidin-3-yl)-1,3-dioxoisoindolin-4- |
| | yl)amino)butyl)-2-(4-(((5-fluoro-4-oxo-2-(((tetrahydro-2H-pyran- |
| | 4-yl)thio)methyl)-3,4-dihydroquinazolin-7- |
| | yl)oxy)methyl)piperidin-1-yl)acetamide |
| 9 | 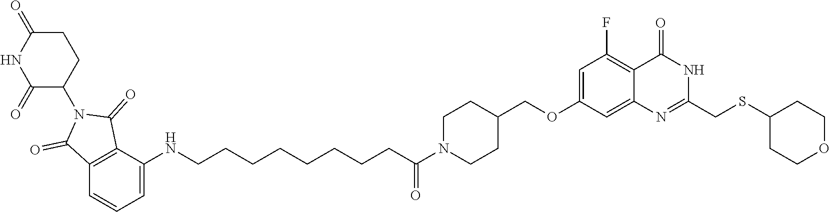 [SMILES: O=C1CCC(N2C(=O)c3cccc(NCCCCCCCCC(=O)N4CCC(COc5cc(F)c6c(=O)[nH]c(CSC7CCOCC7)nc6c5)CC4)c3C2=O)C(=O)N1] |
| | 2-(2,6-dioxopiperidin-3-yl)-4-((9-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-9-oxononyl)amino)isoindoline-1,3- |
| | dione |
| 10 | 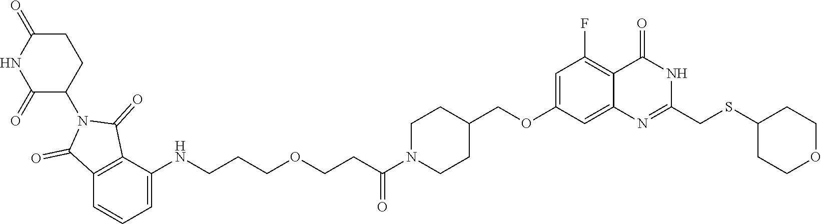 [SMILES: O=C1CCC(N2C(=O)c3cccc(NCCCOCCC(=O)N4CCC(COc5cc(F)c6c(=O)[nH]c(CSC7CCOCC7)nc6c5)CC4)c3C2=O)C(=O)N1] |
| | 2-(2,6-dioxopiperidin-3-yl)-4-((3-(3-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-3- |
| | oxopropoxy)propyl)amino)isoindoline-1,3-dione |
| 11 | 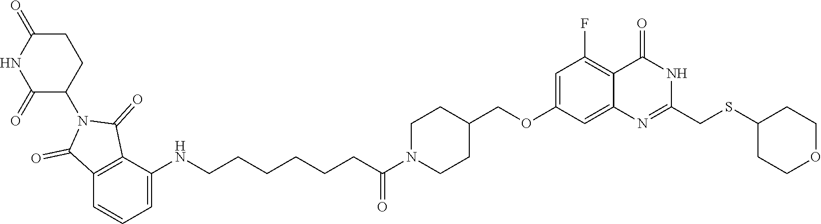 [SMILES: O=C1CCC(N2C(=O)c3cccc(NCCCCCCC(=O)N4CCC(COc5cc(F)c6c(=O)[nH]c(CSC7CCOCC7)nc6c5)CC4)c3C2=O)C(=O)N1] |
| | 2-(2,6-dioxopiperidin-3-yl)-4-((7-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-7-oxoheptyl)amino)isoindoline-1,3- |
| | dione |
| 12 | 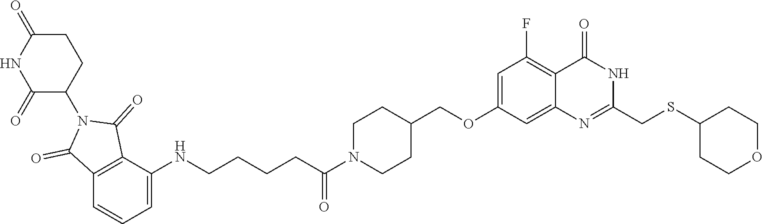 [SMILES: O=C1CCC(N2C(=O)c3cccc(NCCCCC(=O)N4CCC(COc5cc(F)c6c(=O)[nH]c(CSC7CCOCC7)nc6c5)CC4)c3C2=O)C(=O)N1] |
| | 2-(2,6-dioxopiperidin-3-yl)-4-((5-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-5-oxopentyl)amino)isoindoline-1,3- |
| | dione |
| 13 | 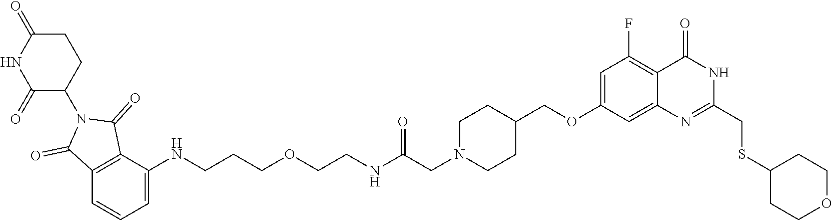 [SMILES: O=C(CN1CCC(COc2cc(F)c3c(=O)[nH]c(CSC4CCOCC4)nc3c2)CC1)NCCOCCCNc1cccc2c1C(=O)N(C1CCC(=O)NC1=O)C2=O] |
| | N-(2-(3-((2-(2,6-dioxopiperidin-3-yl)-1,3-dioxoisoindolin-4- |
| | yl)amino)propoxy)ethyl)-2-(4-(((5-fluoro-4-oxo-2-(((tetrahydro- |
| | 2H-pyran-4-yl)thio)methyl)-3,4-dihydroquinazolin-7- |
| | yl)oxy)methyl)piperidin-1-yl)acetamide |
| 14 | 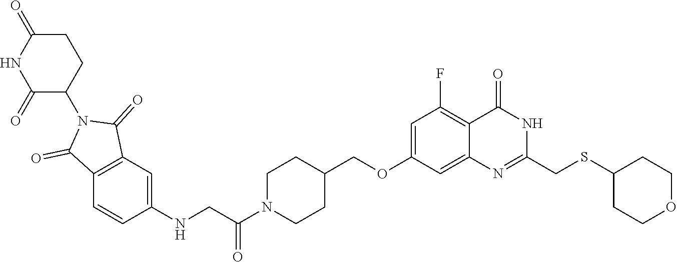 [SMILES: O=C1CCC(N2C(=O)c3ccc(NCC(=O)N4CCC(COc5cc(F)c6c(=O)[nH]c(CSC7CCOCC7)nc6c5)CC4)cc3C2=O)C(=O)N1] |
| | 2-(2,6-dioxopiperidin-3-yl)-5-((2-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)-2-oxoethyl)amino)isoindoline-1,3- |
| | dione |
| 15 | 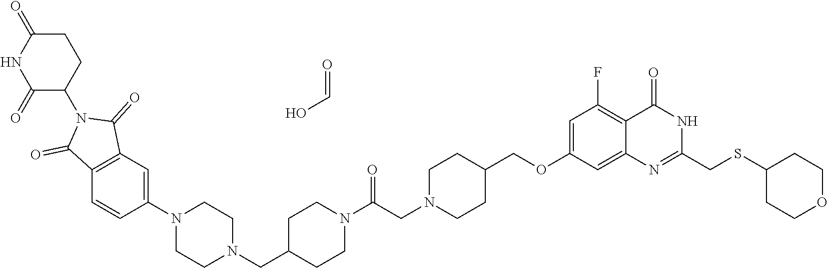 [SMILES: O=C1CCC(N2C(=O)c3ccc(N4CCN(CC5CCN(C(=O)CN6CCC(COc7cc(F)c8c(=O)[nH]c(CSC9CCOCC9)nc8c7)CC6)CC5)CC4)cc3C2=O)C(=O)N1 ; O=CO] |
| | 2-(2,6-dioxopiperidin-3-yl)-5-(4-((1-(2-(4-(((5-fluoro-4-oxo-2- |
| | (((tetrahydro-2H-pyran-4-yl)thio)methyl)-3,4-dihydroquinazolin-7- |
| | yl)oxy)methyl)piperidin-1-yl)acetyl)piperidin-4- |
| | yl)methyl)piperazin-1-yl)isoindoline-1,3-dione formate |
| 16 | 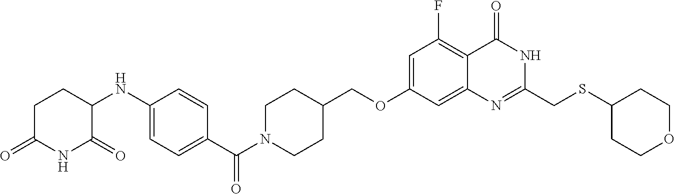 [SMILES: O=C1CCC(Nc2ccc(C(=O)N3CCC(COc4cc(F)c5c(=O)[nH]c(CSC6CCOCC6)nc5c4)CC3)cc2)C(=O)N1] |
| | 3-((4-(4-(((5-fluoro-4-oxo-2-(((tetrahydro-2H-pyran-4- |
| | yl)thio)methyl)-3,4-dihydroquinazolin-7-yl)oxy)methyl)piperidine- |
| | 1-carbonyl)phenyl)amino)piperidine-2,6-dione |
| 17 | 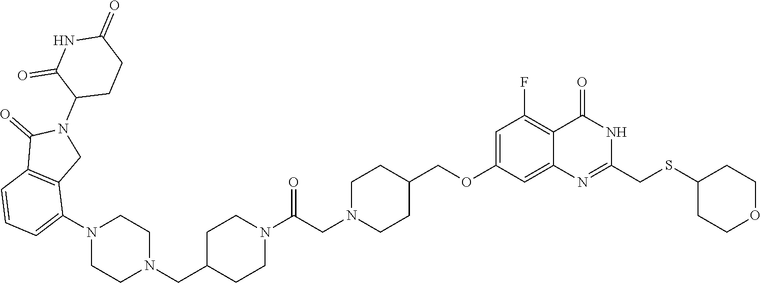 [SMILES: O=C1CCC(N2Cc3c(cccc3N3CCN(CC4CCN(C(=O)CN5CCC(COc6cc(F)c7c(=O)[nH]c(CSC8CCOCC8)nc7c6)CC5)CC4)CC3)C2=O)C(=O)N1] |
| | 3-(4-(4-((1-(2-(4-(((5-fluoro-4-oxo-2-(((tetrahydro-2H-pyran-4- |
| | yl)thio)methyl)-3,4-dihydroquinazolin-7-yl)oxy)methyl)piperidin- |
| | 1-yl)acetyl)piperidin-4-yl)methyl)piperazin-1-yl)-1-oxoisoindolin- |
| | 2-yl)piperidine-2,6-dione |
| 18 | 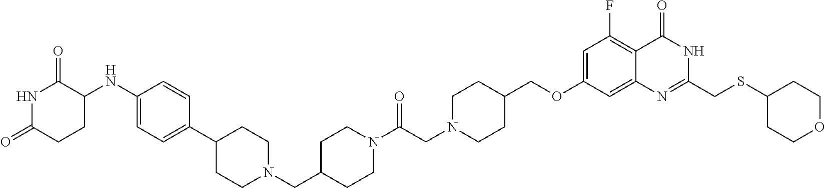 [SMILES: O=C1CCC(Nc2ccc(C3CCN(CC4CCN(C(=O)CN5CCC(COc6cc(F)c7c(=O)[nH]c(CSC8CCOCC8)nc7c6)CC5)CC4)CC3)cc2)C(=O)N1] |
| | 3-((4-(1-((1-(2-(4-(((5-fluoro-4-oxo-2-(((tetrahydro-2H-pyran-4- |
| | yl)thio)methyl)-3,4-dihydroquinazolin-7-yl)oxy)methyl)piperidin- |
| | 1-yl)acetyl)piperidin-4-yl)methyl)piperidin-4- |
| | yl)phenyl)amino)piperidine-2,6-dione |
| 19 | 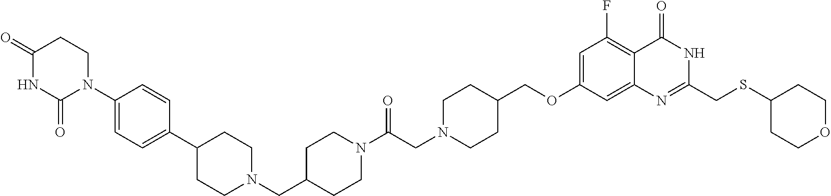 [SMILES: O=C1CCN(c2ccc(C3CCN(CC4CCN(C(=O)CN5CCC(COc6cc(F)c7c(=O)[nH]c(CSC8CCOCC8)nc7c6)CC5)CC4)CC3)cc2)C(=O)N1] |
| | 1-(4-(1-((1-(2-(4-(((5-fluoro-4-oxo-2-(((tetrahydro-2H-pyran-4- |
| | yl)thio)methyl)-3,4-dihydroquinazolin-7-yl)oxy)methyl)piperidin- |
| | 1-yl)acetyl)piperidin-4-yl)methyl)piperidin-4- |
| | yl)phenyl)dihydropyrimidine-2,4(1H,3H)-dione |
| 20 | 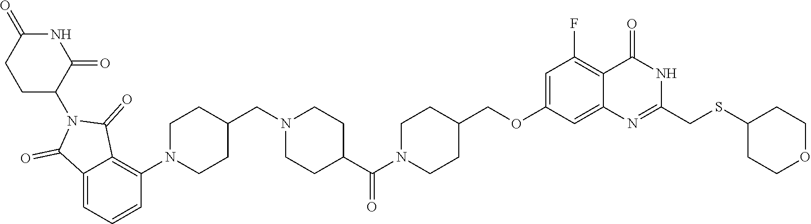 [SMILES: O=C1CCC(N2C(=O)c3cccc(N4CCC(CN5CCC(C(=O)N6CCC(COc7cc(F)c8c(=O)[nH]c(CSC9CCOCC9)nc8c7)CC6)CC5)CC4)c3C2=O)C(=O)N1] |
| | 2-(2,6-dioxopiperidin-3-yl)-4-(4-((4-(4-(((5-fluoro-4-oxo-2- |
| | (((tetrahydro-2H-pyran-4-yl)thio)methyl)-3,4-dihydroquinazolin-7- |
| | yl)oxy)methyl)piperidine-1-carbonyl)piperidin-1- |
| | yl)methyl)piperidin-1-yl)isoindoline-1,3-dione |
| Ex- | |
| am- | |
| ple | Analytical Data |
| 2 | LCMS (ESI, m/z): 764.30 [M + H]⁺. ¹HNMR (400 MHz, DMSO-d₆) δ 12.15 (s, 1H), 11.08 (s, 1H), 7.91 (t, J = 5.8 Hz, 1H), 7.58 (dd, J = 8.6, |
| | 7.1 Hz, 1H), 7.21 (d, J = 8.6 Hz, 1H), 7.03 (d, J = 7.0 Hz, 1H), 6.92-6.80 (m, 2H), 6.71 (t, J = 6.0 Hz, 1H), 5.04 (dd, J = 12.5, 5.4 Hz, 1H), |
| | 3.95 (d, J = 6.0 Hz, 2H), 3.81 (dd, J = 9.5, 5.8 Hz, 2H), 3.62 (s, 2H), 3.54-3.40 (m, 6H), 3.15-3.06 (m, 1H), 2.9-2.77 (m, 5H), 2.67-2.55 |
| | (m, 2H), 2.08-1.99 (m, 3H), 1.89-1.81 (m, 2H), 1.75-1.65 (m, 3H), 1.47-1.21 (m, 4H). |
| 3 | LCMS (ESI, m/z): 848.40 [M + H]⁺. ¹HNMR (300 MHz, DMSO-d₆) δ 12.14 (s, 1H), 11.08 (s, 1H), 7.74-7.42 (m, 2H), 7.01 (dd, J = 17.1, |
| | 7.8 Hz, 2H), 6.84 (d, J = 10.9 Hz, 2H), 6.48 (t, J = 5.9 Hz, 1H), 5.03 (dd, J = 12.8, 5.4 Hz, 1H), 3.94 (d, J = 5.8 Hz, 2H), 3.85-3.74 (m, 2H), |
| | 3.59 (s, 2H), 3.31-3.23 (m, 4H), 3.09-3.00 (m, 3H), 2.88-2.75 (m, 5H), 2.61-2.55 (m, 2H), 2.16-1.93 (m, 3H), 1.81-1.82 (m, 2H), 1.79-1.68 |
| | (m, 3H), 1.53 (d, J = 7.0 Hz, 2H), 1.48-1.16 (m, 14H). |
| 4 | LCMS (ESI, m/z): 823.40 [M + H]⁺. ¹HNMR (300 MHz, DMSO-d₆) δ 12.16 (s, 1H), 11.10 (s, 1H), 7.59-7.53 (m, 1H), 7.14 (d, J = 8.6 Hz, |
| | 1H), 7.03 (d, J = 7.0 Hz, 1H), 6.91-6.82 (m, 2H), 6.60 (t, J = 5.8 Hz, 1H), 5.09-4.99 (m, 1H), 4.40 (d, J = 15.3 Hz, 1H), 3.97 (d, J = 6.01 Hz, |
| | 2H), 3.96-3.83 (m, 1H), 3.88-3.72 (m, 2H), 3.69-3.58 (m, 6H), 3.57-3.49 (m, 4H), 3.48-3.40 (m, 2H), 3.35-3.33 (m, 1H), 3.27-3.25 (m, 1H), |
| | 3.11-3.01 (m, 2H), 3.00-2.91 (m, 1H), 2.90-2.81 (m, 1H), 2.68-2.50 (m, 4H), 2.09-1.92 (m, 2H), 1.91-1.81 (m, 2H), 1.80-1.69 (m, 2H), 1.52- |
| | 1.33 (m, 2H), 1.31-1.01 (m, 2H) |
| 5 | LCMS (ESI, m/z): 735.40 [M + H]⁺. ¹HNMR (300 MHz, DMSO-d₆) δ 12.20 (s, 1H) 11.10 (s, 1H), 7.60 (dd, J = 8.6, 7.1 Hz, 1H), 7.16 (d, J = |
| | 8.6 Hz, 1H), 7.03 (d, J = 7.0 Hz, 1H), 6.89 (d, J = 9.0 Hz, 2H), 6.78 (t, J = 6.3 Hz, 1H), 5.05 (dd, J = 12.8, 5.4 Hz, 1H), 4.45 (d, J = 12.9 Hz, |
| | 1H), 4.02-3.73 (m, 6H), 3.62-3.49 (m, 3H), 3.12-2.95 (m, 2H), 2.93-2.80 (m, 2H), 2.74-2.59 (m, 4H), 2.17-1.95 (m, 3H), 1.90-1.72 (M, 4H), |
| | 1.54-1.35 (m, 2H), 1.30-1.11 (m, 3H). |
| 6 | LCMS (ESI, m/z): 721.25 [M + H]⁺. ¹HNMR (400 MHz, DMSO-d₆) δ 12.15 (s, 1H), 11.11 (s, 1H), 7.61 (dd, J = 8.5, 7.1 Hz, 1H), 7.27-6.98 |
| | (m, 3H), 6.90 (d, J = 10.3 Hz, 2H), 5.07 (dd, J = 12.9, 5.4 Hz, 1H), 4.44 (d, J = 12.9 Hz, 1H), 4.26-4.14 (m, 2H), 4.02 (d, J = 6.4 Hz, 2H), |
| | 3.99-3.89 (m, 1H), 3.87-3.70 (m, 2H), 3.62 (s, 2H), 3.53-3.40 (m, 1H), 3.30-3.21 (m, 1H), 3.15-3.01 (m, 2H), 2.95-2.80 (m, 1H), 2.75- |
| | 2.68 (m, 1H), 2.65-2.54 (m, 2H), 2.15-2.03 (m, 2H), 1.87-1.65 (m, 4H), 1.46-1.35 (m, 2H), 1.31-1.22 (m, 1H), 1.21-1.14 (m, 1H). |
| 7 | LCMS (ESI, m/z): 852.40 [M + H]⁺. ¹HNMR (300 MHz, DMSO-d₆) δ 11.34 (s, 1H), 7.65 (t, J = 5.4 Hz, 1H), 7.56 (t, J = 7.8 Hz, 1H), 7.21- |
| | 7.18 (m, 1H), 7.16-6.98 (m, 1H), 6.85 (s, 1H), 6.83 (s, 1H), 6.59 (t, J = 5.7 Hz, 1H), 5.06 (dd, J = 12.6, 5.4 Hz, 1H), 3.93 (d, J = 5.8 Hz, 2H), |
| | 3.88-3.72 (m, 2H), 3.67-3.61 (m, 4H), 3.59-3.41 (m, 9H), 3.3-3.18 (m, 3H), 3.11-2.98 (m, 1H), 2.96-2.79 (m, 5H), 2.69-2.55 (m, 2H), 2.14- |
| | 1.97 (m, 3H), 1.95-1.81 (m, 2H), 1.80-1.60 (m, 3H), 1.56-1.20 (m, 4H). |
| 8 | LCMS (ESI, m/z): 792.35 [M + H]⁺. ¹HNMR (300 MHz, DMSO-d₆) δ 12.18 (s, 1H), 11.08 (s, 1H), 7.62-7.51 (m, 1H), 7.09 (d, J = 8.5 Hz, 1H), |
| | 7.00 (d, J = 7.1 Hz, 1H), 6.86 (d, J = 13.5 Hz, 2H), 6.55 (s, 1H), 5.03 (dd, J = 12.9, 5.3 Hz, 1H), 4.03-3.97 (m, 2H), 3.80 (d, J = 11.8 Hz, 2H), |
| | 3.61 (s, 2H), 3.35-3.25 (m, 2H), 3.19-2.80 (m, 8H), 2.60-2.50 (m, 3H), 2.08-1.75 (m, 7H), 1.73-1.28 (m, 8H). |
| 9 | LCMS (ESI, m/z): 819.35 [M + H]⁺. ¹HNMR (300 MHz, DMSO-d₆) δ 12.10 (s, 1H), 11.09 (s, 1H), 7.56 (dd, J = 8.6, 7.1 Hz, 1H), 7.2-6.97 |
| | (m, 2H), 6.94-6.76 (m, 2H), 6.50 (t, J = 6.0 Hz, 1H), 5.03 (dd, J = 12.8, 5.4 Hz, 1H), 4.40 (d, J = 13.0 Hz, 1H), 4.06-3.98 (m, 2H), 3.96- |
| | 3.72 (m, 3H), 3.69-3.55 (m, 2H), 3.32-3.25 (m, 4H), 3.11-2.81 (m, 3H), 2.63-2.55 (m, 2H), 2.34-2.21 (m, 2H), 2.12-2.09 (m, 0.4H), 2.06-1.96 |
| | (m, 2H), 1.94-1.7 (m, 4H), 1.64-1.41 (m, 6H), 1.4-1.21 (m, 9H), 1.2-1 (m, 2H). |
| 10 | LCMS (ESI, m/z): 793.40 [M + H]⁺. ¹HNMR (300 MHz, DMSO-d₆) δ 9.60 (s, 1H), 7.58 (dd, J = 8.6, 7.1 Hz, 1H), 7.09 (d, J = 8.6 Hz, 1H), |
| | 7.02 (d, J = 7.0 Hz, 1H), 6.89 (s, 1H), 6.85 (d, J = 2.4 Hz, 1H), 6.71 (t, J = 5.8 Hz, 1H), 5.05 (dd, J = 12.8, 5.3 Hz, 1H), 4.42 (d, J = 13.0 Hz, |
| | 1H), 4.05-3.92 (m, 3H), 3.84-3.78 (m, 2H), 3.69-3.58 (m, 4H), 3.50-3.40 (m, 4H), 3.04-2.80 (m, 3H), 2.68-2.55 (m, 1H), 2.46-2.40 (m, 1H), |
| | 2.14-1.98 (m, 2H), 1.87-1.73 (m, 6H), 1.54-1.35 (m, 2H), 1.24-1.13 (m, 2H). |
| 11 | LCMS (ESI, m/z): 791.35 [M + H]⁺. ¹HNMR (300 MHz, DMSO-d₆) δ 12.14 (s, 1H), 11.08 (s, 1H), 7.56 (dd, J = 8.6, 7.1 Hz, 1H), 7.08 (d, J = |
| | 8.6 Hz, 1H), 7.00 (d, J = 7.0 Hz, 1H), 6.88 (d, J = 2.6 Hz, 1H), 6.84 (d, J = 2.4 Hz, 1H), 6.53 (t, J = 5.9 Hz, 1H), 5.03 (dd, J = 12.8, 5.4 Hz, |
| | 1H), 4.40 (d, J = 13.0 Hz, 1H), 3.98 (d, J = 6.3 Hz, 2H), 3.93-3.75 (m, 3H), 3.60 (s, 2H), 3.27 (s, 2H), 3.10-2.85 (m, 5H), 265-2.55 (m, 3H), |
| | 2.31-2.23 (m, 3H), 2.10-1.94 (m, 2H), 1.92-1.68 (m, 4H), 1.62-1.37 (m, 6H), 1.36-1.25 (m, 4H), 1.24-1.01 (m, 3H). |
| 12 | LCMS (ESI, m/z): 763.30 [M + H]⁺. ¹HNMR (400 MHz, DMSO-d₆) δ 12.09 (s, 1H), 11.09 (s, 1H), 7.58 (dd, J = 8.6, 7.0 Hz, 1H), 7.11 (d, J = |
| | 8.6 Hz, 1H), 7.02 (d, J = 7.0 Hz, 1H), 6.87 (d, J = 10.7 Hz, 2H), 6.56 (t, J = 6.0 Hz, 1H), 5.04 (dd, J = 13.0, 5.4 Hz, 1H), 4.41 (d, J = 13.0 Hz, |
| | 1H), 3.98 (d, J = 6.3 Hz, 2H), 3.90 (d, J = 13.5 Hz, 1H), 3.89-3.80 (m, 2H), 3.72- (s, 2H), 3.29-3.15 (m, 3H), 3.12-2.97 (m, 2H), 2.94-2.81 (m, |
| | 1H), 2.62-2.55 (m, 3H), 2.43-2.29 (m, 2H), 2.10-1.96 (m, 2H), 1.93-1.83 (m, 2H), 1.82-1.74 (m, 2H), 1.65-1.53 (m, 4H), 1.47-1.29 (m, 2H), |
| | 1.27-1.01 (m, 3H). |
| 13 | LCMS (ESI, m/z): 822.35 [M + H]⁺. ¹HNMR (300 MHz, DMSO-d₆) δ 11.95 (s, 1H), 11.09 (s, 1H), 7.69 (t, J = 5.9 Hz, 1H), 7.56 (dd, J = 8.5, |
| | 7.1 Hz, 1H), 7.08 (d, J = 8.6 Hz, 1H), 6.98 (d, J = 7.0 Hz, 1H), 6.80 (dd, J = 12.4, 1.2 Hz, 2H), 6.74 (d, J = 5.9 Hz, 1H), 5.04 (dd, J = 12.8, |
| | 5.4 Hz, 1H), 3.94-3.74 (m, 4H), 3.62 (s, 2H), 3.52 (t, J = 5.7 Hz, 2H), 3.47-3.37 (m, 3H), 3.36 (s, 2H), 3.29 (s, 1H), 3.12-3.01 (m, 1H), 2.89 |
| | (s, 3H), 2.81-2.75 (m, 2H), 2.65-2.52 (m, 3H), 2.08-1.91 (m, 3H), 1.90-1.75 (m, 4H), 1.74-1.62 (m, 3H), 1.53-1.39 (m, 2H), 1.35-1.18 (m, 3H). |
| 14 | LCMS (ESI, m/z): 721.40 [M + H]⁺. ¹HNMR (300 MHz, DMSO-d₆) δ 12.15 (s, 1H), 11.06 (s, 1H), 7.58 (d, J = 8.4 Hz, 1H), 7.14 (s, 1H), 7.11- |
| | 6.99 (m, 2H), 6.89-6.92 (m, 2H), 5.05 (dd, J = 12.6, 5.4 Hz, 1H), 4.43 (d, J = 13.1 Hz, 1H), 4.14 (t, J = 5.7 Hz, 1H), 4.10-3.99 (m, 3H), 3.82- |
| | 3.85 (m, 2H), 3.62 (s, 2H), 3.36-3.31 (m, 1H), 3.15-2.99 (m, 2H), 2.94-2.78 (m, 1H), 2.76-2.55 (m, 3H), 2.15-1.97 (m, 2H), 1.77-1.94 (m, 3H), |
| | 1.57-1.14 (m, 4H). |
| 15 | LCMS (ESI, m/z): 887.40 [M + H]⁺. ¹HNMR (400 MHz, DMSO-d₆) δ 12.13 (s, 1H), 11.08 (s, 1H), 8.20 (s, 1H, HCOOH), 7.68 (d, J = 8.5 Hz, |
| | 1H), 7.34 (d, J = 2.3 Hz, 1H), 7.26 (dd, J = 8.7, 2.3 Hz, 1H), 6.88 (dd, J = 11.3, 2.0 Hz, 2H), 5.08 (dd, J = 12.9, 5.4 Hz, 1H), 4.33 (d, J = 12.7 |
| | Hz, 1H), 4.07 (d, J = 12.8 Hz, 1H), 3.99 (d, J = 6.0 Hz, 2H), 3.86-3.77 (m, 2H), 3.62 (s, 2H), 3.41-3.29 (m, 4H), 3.38-3.27 (m, 4H), 3.22-3.15 |
| | (m, 1H), 3.12-2.91 (m, 4H), 2.90-2.78 (m, 3H), 2.63-2.48 (m, 4H), 2.19 (d, J = 6.9 Hz, 2H), 2.10-1.96 (m, 3H), 1.94-1.84 (m, 2H), 1.81-1.68 |
| | (m, 6H), 1.52-1.38 (m, 2H), 1.37-1.22 (m, 2H), 1.08-0.83 (m, 2H). |
| 16 | LCMS (ESI, m/z): 638.25 [M + H]⁺. ¹HNMR (300 MHz, DMSO-d₆) δ 12.14 (s, 1H), 10.81 (s, 1H), 7.18 (d, J = 8.3 Hz, 2H), 6.97-6.77 (m, |
| | 2H), 6.69 (d, J = 8.4 Hz, 2H), 6.28 (d, J = 7.8 Hz, 1H), 4.52-4.31 (m, 1H), 4.20-4.14 (m, 1H), 4.07-4.00 (m, 2H), 3.86-3.78 (m, 2H), 3.62 (s, |
| | 2H), 3.14-3.01 (m, 2H), 3.01-2.85 (m, 2H), 2.83-2.70 (m, 1H), 2.62 (s, 1H), 2.59-2.54 (m, 1H), 2.09 (s, 2H), 1.95-1.85 (m, 3H), 1.84-1.75 m, |
| | 2H), 1.55-1.37 (m, 2H), 1.37-1.16 (m, 3H). |
| 17 | LCMS (ESI, m/z): 873.40 [M + H]⁺. ¹HNMR (400 MHz, DMSO-d₆) δ 12.11 (s, 1H), 10.98 (s, 1H), 7.44 (t, J = 7.7 Hz, 1H), 7.32 (d, J = |
| | 7.3 Hz, 1H), 7.16 (d, J = 8.0 Hz, 1H), 6.88 (d, J = 10.1 Hz, 2H), 5.12 (dd, J = 13.2, 5.1 Hz, 1H), 4.51-4.45 (m, 1H), 4.37-4.26 (m, 2H), 4.08 |
| | (d, J = 12.9 Hz, 1H), 3.99 (d, J = 5.9 Hz, 2H), 3.85-3.78 (m, 2H), 3.62 (s, 2H), 3.30-3.21 (m, 2H), 3.11-2.78 (m, 11H), 2.55-2.40 (m, |
| | 3H), 2.20 (d, J = 6.9 Hz, 2H), 2.05-1.97 (m, 4H), 1.88-1.80 (m, 2H), 1.76-1.68 (m, 6H), 1.52-1.38 (m, 2H), 1.34-1.22 |
| | (m, 2H), 1.08-0.84 (m, 4H). |
| 18 | LCMS (ESI, m/z): 832.45 [M + H]⁺. ¹HNMR (300 MHz, DMSO-d₆) δ 12.12 (s, 1H), 10.76 (s, 1H), 7.14-6.79 (m, 4H), 6.59 (d, J = 8.3 Hz, 2H), |
| | 5.62 (d, J = 7.4 Hz, 1H), 4.39-4.18 (m, 2H), 4.00 (dd, J = 26.5, 9.4 Hz, 3H), 3.80 (dd, J = 11.5, 3.7 Hz, 2H), 3.60 (s, 2H), 3.34-3.19 (m, 6H), |
| | 3.10-2.56 (m, 8H), 2.45-2.41 (m, 2H), 2.30-2.13 (m, 1H), 2.09-2.01 (m, 2H), 1.96-1.24 (m, , 19H), 1.10-0.99 (m, 1H), 0.91-0.79 (m, 1H). |
| 19 | LCMS (ESI, m/z): 818.45 [M + H]⁺. ¹HNMR (400 MHz, DMSO-d₆) δ 12.09 (s, 1H), 10.33 (s, 1H), 7.30-7.20 (m, 4H), 6.87 (d, J = 11.3 |
| | Hz, 2H), 4.33 (d, J = 12.9 Hz, 1H), 4.07 (d, J = 13.0 Hz, 1H), 3.98 (d, J = 5.9 Hz, 2H), 3.85-3.75 (m, 4H), 3.62 (s, 2H), 3.37-3.33 (m, 2H), |
| | 3.31-3.29 (m, 1H), 3.24-3.17 (m, 1H), 3.11-2.89 (m, 5H), 2.89-2.79 (m, 2H), 2.69 (t, J = 6.6 Hz, 2H), 2.60-2.53 (m, 1H), 2.49-2.36 (m, 1H), |
| | 2.15 (d, J = 6.8 Hz, 2H), 2.07-1.92 (m, 4H), 1.92-1.84 (m, 2H), 1.84-1.70 (m, 7H), 1.70-1.58 (m, 2H), 1.51-1.39 (m, 2H), |
| | 1.37-1.22 (m, 2H), 1.12-0.99 (m, 1H), 0.97-0.80 (m, 1H). |
| 20 | LCMS (ESI, m/z): 872.45 [M + H]⁺. ¹HNMR (300 MHz, DMSO-d₆) δ 12.15 (s, 1H), 11.09 (s, 1H), 7.68 (dd, J = 8.5, 7.1 Hz, 1H), 7.33 (dd, J = |
| | 7.9, 6.0 Hz, 2H), 6.96-6.82 (m, 2H), 5.09 (dd, J = 12.7, 5.4 Hz, 1H), 4.43 (d, J = 12.8 Hz, 1H), 4.09-3.91 (m, 3H), 3.89-3.77 (m, 2H), 3.69 (d, |
| | J = 11.8 Hz, 2H), 3.62 (s, 2H), 3.37-3.27 (m, 3H), 3.12-2.97 (m, 2H), 2.94-2.80 (m, 5H), 2.65-2.59 (m, 3H), 2.17 (d, J = 6.8 Hz, 2H), 2.11- |
| | 1.01 (m, 21H). |

### Example 21: Synthesis of 2-(2,6-dioxopiperidin-3-yl)-4-(4-((l-(2-(4-(((5-fluoro-4-oxo-2-(((tetrahydro-2H-pyran-4-yl)thio)methyl)-3,4-dihydroquinazolin-7-yl)oxy)methyl)piperidin-1l-yl)acetyl)piperidin-4-yl)methyl)piperazin-1-yl)isoindoline-1,3-dione

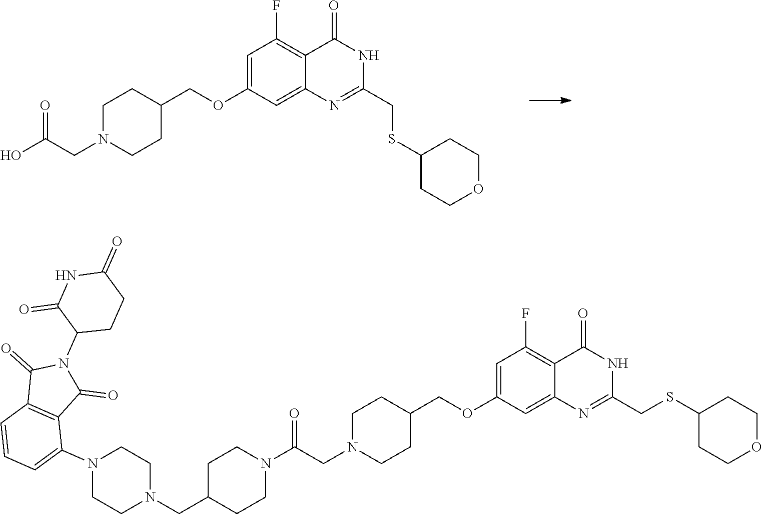 [SMILES: O=C(O)CN1CCC(COc2cc(F)c3c(=O)[nH]c(CSC4CCOCC4)nc3c2)CC1 ; O=C1CCC(N2C(=O)c3cccc(N4CCN(CC5CCN(C(=O)CN6CCC(COc7cc(F)c8c(=O)[nH]c(CSC9CCOCC9)nc8c7)CC6)CC5)CC4)c3C2=O)C(=O)N1]

A solution of 2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetic acid (3.0 g, 6.44 mmol, 1.0 equiv), 2-(2,6-dioxopiperidin-3-yl)-4-(4-(piperidin-4-ylmethyl)piperazin-1-yl)isoindoline-1,3-dione (2.74 g, 6.44 mmol, 1.0 equiv), TCFH (2.71 g, 9.67 mmol, 1.5 equiv) and NMI (2.65 g, 32.2 mmol, 0.05 equiv) in MeCN (50 mL) was stirred for 2 hours. The reaction is concentrated then the residue was purified by reverse flash chromatography with the following conditions: (column, C18 silica gel; mobile phase, MeCN in water (10 mmol/L HCOOH), 10% to 45% gradient in 30 min; detector, UV 254 nm) to 2-(2,6-dioxopiperidin-3-yl)-4-(4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperazin-1-yl)isoindoline-1,3-dione (2.2 g, 95%) as a green solid. LCMS (ESI, m/z): 887.04 [M+H]⁺. ¹H NMR (300 MHz, DMSO-d₆) δ 12.18 (s, 1H), 11.11 (s, 1H), 8.15 (s, 3H, HCOOH), 7.71 (m, 1H), 7.35 (t, J=7.8 Hz, 2H), 6.89-6.81 (m, 2H), 5.12-5.04 (m, 1H), 4.34 (d, J=12.6 Hz, 1H), 4.08-3.95 (m, 3H), 3.82-3.75 (m, 2H), 3.61 (s, 2H), 3.45-3.13 (m, 8H), 3.12-2.83 (m, 6H), 2.58-2.51 (m, 4H), 2.20 (d, J=6.8 Hz, 2H), 2.11-1.96 (m, 3H), 1.88-1.75 (m, 8H), 1.55-1.17 (m, 4H), 1.19-0.82 (m, 2H).

### Example 22: Synthesis of 2-(2,6-dioxopiperidin-3-yl)-4-((2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethoxy)ethyl)amino)isoindoline-1,3-dione

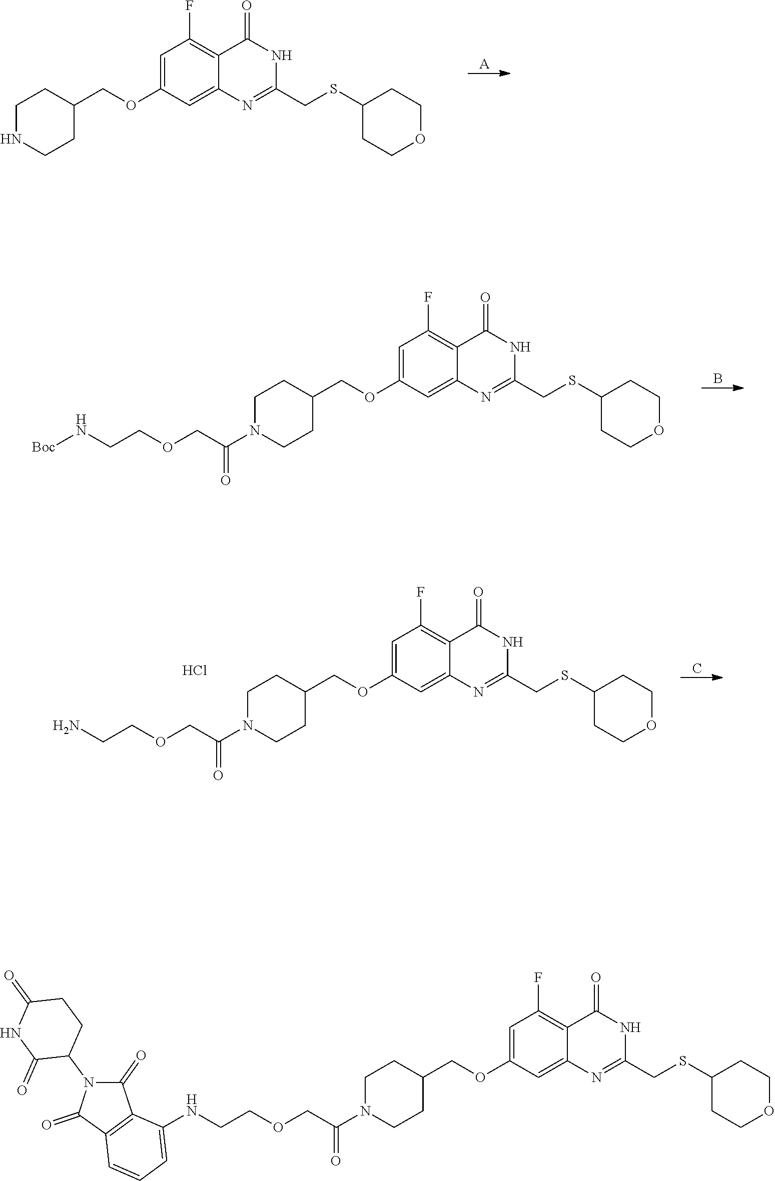

### Step A

A solution of 5-fluoro-7-(piperidin-4-ylmethoxy)-2-(((tetrahydro-2H-pyran-4-yl)thio)methyl)quinazolin-4(3H)-one (557 mg, 1.37 mmol, 1.0 equiv), 2-(2-((tert-butoxycarbonyl)amino)ethoxy)acetic acid (557 mg, 2.54 mmol, 1.86 equiv), DIEA (707 mg, 5.47 mmol, 4.0 equiv) and HATU (780 mg, 2.05 mmol, 1.5 equiv) in DMF (5 mL) was stirred for 2 hours. The mixture was purified by reversed-phase flash chromatography with the following conditions: (column, C18 silica gel; mobile phase, MeCN in water (10 mmol/L NH₄HCO₃), 10% to 50% gradient in 10 min; detector, UV 254 nm) to afford tert-butyl (2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethoxy)ethyl)carbamate (560 mg, 68%) as a brown solid. LCMS (ESI, m/z): 609.25 [M+H]⁺.

### Step B

A solution of tert-butyl (2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethoxy)ethyl)carbamate (560 mg, 0.92 mmol, 1.0 equiv) and HCl in 1,4-dioxane (30 mL, 4 M) was stirred for 1 hour. The resulting mixture was concentrated under reduced pressure to afford 7-((1-(2-(2-aminoethoxy)acetyl)piperidin-4-yl)methoxy)-5-fluoro-2-(((tetrahydro-2H-pyran-4-yl)thio)methyl)quinazolin-4(3H)-one hydrochloride (430 mg, 86%) as a crude brown solid. that was used without further purification. LCMS (ESI, m/z): 509.45 [M+H]⁺.

### Step C

A solution of 7-((1-(2-(2-aminoethoxy)acetyl)piperidin-4-yl)methoxy)-5-fluoro-2-(((tetrahydro-2H-pyran-4-yl)thio)methyl)quinazolin-4(3H)-one hydrochloride (410 mg, 0.75 mmol, 1.5 equiv), 2-(2,6-dioxopiperidin-3-yl)-4-fluoroisoindoline-1,3-dione (139 mg, 0.50 mmol, 1.0 equiv) and TEA (152 mg, 1.50 mmol, 3.0 equiv) in NMP (5 mL) was stirred for 1 hour at 100° C. The reaction mixture was purified by reversed-phase flash chromatography and further purified with the following conditions: (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 21% B to 41% B in 11 min, 41% B; Wave Length: 220/254 nm; RT1 (min): 12.92) to afford 2-(2,6-dioxopiperidin-3-yl)-4-((2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethoxy)ethyl)amino)isoindoline-1,3-dione (21.8 mg, 6%) as a yellow green solid. LCMS (ESI, m/z): 765.25 [M+H]⁺. ¹HNMR (400 MHz, DMSO-d₆) δ 12.17 (s, 1H), 11.09 (s, 1H), 7.58 (t, J=7.8 Hz, 1H), 7.15 (d, J=8.6 Hz, 1H), 7.03 (d, J=7.0 Hz, 1H), 6.86-6.75 (m, 2H), 6.68 (t, J=5.8 Hz, 1H), 5.05 (dd, J=12.9, 5.4 Hz, 1H), 4.35 (d, J=12.8 Hz, 1H), 4.30-4.12 (m, 2H), 3.94 (d, J=6.4 Hz, 2H), 3.90-3.75 (m, 3H), 3.71-3.56 (m, 4H), 3.55-3.45 (m, 2H), 3.29-3.24 (m, 1H), 3.09-2.80 (m, 3H), 2.66-2.52 (m, 3H), 2.02 (dd, J=13.2, 6.8 Hz, 2H), 1.93-1.84 (m, 2H), 1.80-1.69 (m, 2H), 1.51-1.38 (m, 2H), 1.35-1.01 (m, 3H).

### Example 23: Synthesis of 2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione

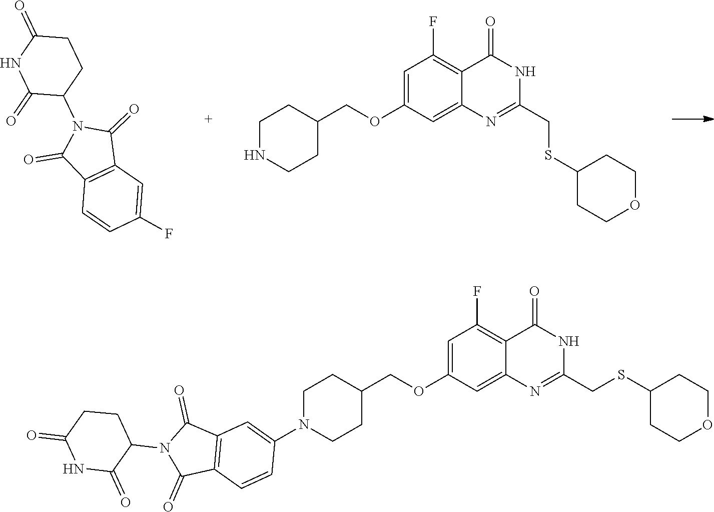 [SMILES: O=C1CCC(N2C(=O)c3ccc(F)cc3C2=O)C(=O)N1 ; "+" ; O=c1[nH]c(CSC2CCOCC2)nc2cc(OCC3CCNCC3)cc(F)c12 ; O=C1CCC(N2C(=O)c3ccc(N4CCC(COc5cc(F)c6c(=O)[nH]c(CSC7CCOCC7)nc6c5)CC4)cc3C2=O)C(=O)N1]

A solution of 2-(2,6-dioxopiperidin-3-yl)-5-fluoroisoindoline-1,3-dione (100 mg, 0.36 mmol, 1.0 equiv), 5-fluoro-7-(piperidin-4-ylmethoxy)-2-(((tetrahydro-2H-pyran-4-yl)thio)methyl)quinazolin-4(3H)-one (148 mg, 0.36 mmol, 1.0 equiv), and DIEA (140 mg, 1.09 mmol, 3.0 equiv) in NMP (5 mL) was stirred for 1 hour at 120° C. The reaction mixture was purified by reverse flash chromatography with the following conditions: (column, C18 silica gel; mobile phase, MeCN in water (0.1% NH₄HCO₃), 0% to 44% gradient in 30 min; detector, UV 254 nm) to afford 2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione (65.3 mg, 27%) as a light yellow solid. LCMS (ESI, m/z): 664.25 [M+H]⁺. ¹HNMR (400 MHz, DMSO-d₆) δ 12.13 (s, 1H), 11.08 (s, 1H), 7.66 (d, J=8.0 Hz, 1H), 7.34 (d, J=4.0 Hz, 1H), 7.26 (dd, J=12.0, 4.0 Hz, 1H), 6.93-6.85 (m, 2H), 5.07 (dd, J=12.0, 8.0 Hz, 1H), 4.11 (d, J=12.0 Hz, 2H), 4.02 (d, J=4.0 Hz, 2H), 3.81-3.76 (m, 2H), 3.61 (s, 2H), 3.38-3.33 (m, 2H), 3.11-3.02 (m, 3H), 2.91-2.82 (m, 1H), 2.63-2.52 (m, 2H), 2.18-1.96 (m, 2H), 1.88 (d, J=12.0 Hz, 4H), 1.51-1.29 (m, 4H).

Example 24 was synthesized according to the procedure described for the synthesis of 2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione (Example 23) using appropriate building blocks and modified reaction conditions (such as reagents, reagent ratio, temperature, and reaction time) and purification conditions as needed.

| Example | Structure | Analytical Data |
|---|---|---|
| 24 | 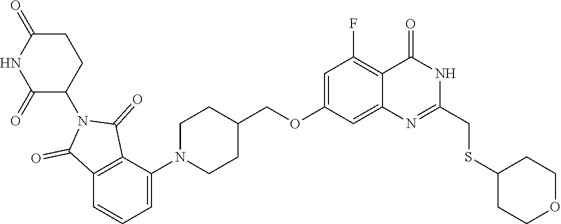 [SMILES: O=C1CCC(N2C(=O)c3cccc(N4CCC(COc5cc(F)c6c(=O)[nH]c(CSC7CCOCC7)nc6c5)CC4)c3C2=O)C(=O)N1] 2-(2,6-dioxopiperidin-3-yl)-4-(4-(((5- | LCMS: 664.25 [M + H]⁺. ¹H NMR (300 MHz, DMSO-d₆) δ 12.16 (s, 1H), 11.09 (s, 1H), 7.70 (dd, J = 8.5, 7.1 Hz, 1H), 7.36 (t, J = 7.9 Hz, 2H), 6.92 (dd, J = 10.8, 2.3 Hz, 2H), 5.10 (dd, J = 12.6, 5.4 Hz, 1H), 4.08 (d, J = 6.1 Hz, 2H), 3.88 − 3.77 (m, 2H), 3.76 − 3.71 (m, 2H), 3.63 (s, 2H), 3.37-3.31 (m, 1H), 3.29- 3.21 (m, 1H), 3.14 − 3.02 (m, 1H), 3.01 − 2.80 (m, 3H), 2.59- 2.51 (m, 2H), 2.11 − 1.99 (m, 2H), 1.92-1.83 (m, 4H), 1.64 − 1.36 (m, 4H). |
| | fluoro-4-oxo-2-(((tetrahydro-2H-pyran-4- | |
| | yl)thio)methyl)-3,4-dihydroquinazolin-7- | |
| | yl)oxy)methyl)piperidin-1-yl)isoindoline- | |
| | 1,3-dione | |

### Example 25: Synthesis of N-(2-(2,6-dioxopiperidin-3-yl)-1,3-dioxoisoindolin-5-yl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide

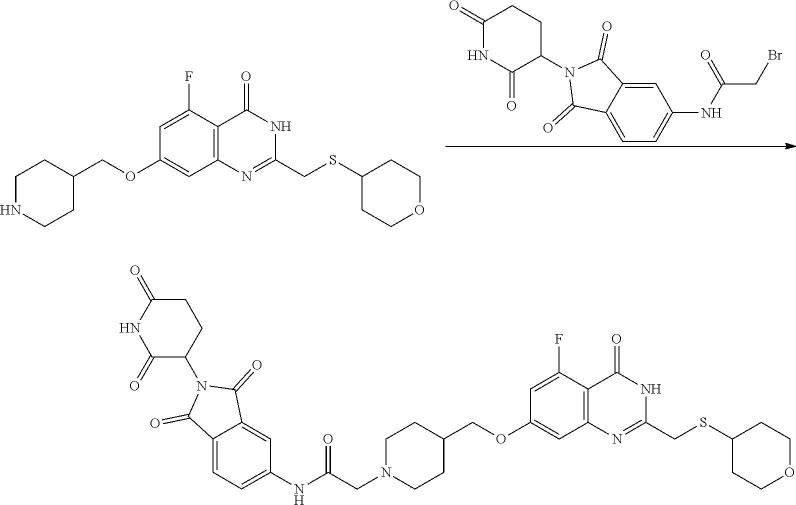 [SMILES: O=c1[nH]c(CSC2CCOCC2)nc2cc(OCC3CCNCC3)cc(F)c12 ; O=C1CCC(N2C(=O)c3ccc(NC(=O)CBr)cc3C2=O)C(=O)N1 ; O=C1CCC(N2C(=O)c3ccc(NC(=O)CN4CCC(COc5cc(F)c6c(=O)[nH]c(CSC7CCOCC7)nc6c5)CC4)cc3C2=O)C(=O)N1]

A solution of 2-bromo-N-[2-(2,6-dioxopiperidin-3-yl)-1,3-dioxoisoindol-5-yl]acetamide (100 mg, 0.25 mmol, 1.0 equiv) and 5-fluoro-2-[(oxan-4-ylsulfanyl)methyl]-7-(piperidin-4-ylmethoxy)-3H-quinazolin-4-one (103 mg, 0.25 mmol, 1.0 equiv), K₂CO₃ (70.1 mg, 0.51 mmol, 2.0 equiv) in DMF (2 mL) was stirred for 1 hour at 70° C. The reaction is diluted with water (10 mL) and extracted with EtOAc (3×10 mL). The combined organic layers were washed with brine (3×10 mL), dried over anhydrous Na₂SO₄, filtered and concentrated under reduced pressure. The crude product was purified by reverse flash chromatography with the following conditions: (column, C18 silica gel; mobile phase, MeCN in water (10 mmol/L NH₄HCO₃), 5% to 50% gradient in 30 min; detector, UV 254 nm) to afford N-(2-(2,6-dioxopiperidin-3-yl)-1,3-dioxoisoindolin-5-yl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide (30.6 mg, 17%) as a white solid. LCMS (ESI, m/z): 721.30 [M+H]⁺. ¹HNMR (300 MHz, DMSO-d₆) δ 12.07 (s, 1H), 11.10 (s, 1H), 10.36 (s, 1H), 8.29 (d, J=1.8 Hz, 1H), 8.08-7.99 (m, 1H), 7.87 (d, J=8.2 Hz, 1H), 6.92-6.83 (m, 2H), 5.12 (dd, J=12.8, 5.3 Hz, 1H), 3.99 (d, J=5.9 Hz, 2H), 3.80 (d, J=11.6 Hz, 2H), 3.60 (s, 2H), 3.53-3.45 (m, 2H), 3.20 (s, 2H), 3.11-3.03 (m, 1H) 2.99-2.87 (m, 3H), 2.61-2.53 (m, 2H), 2.25-2.11 (m, 2H), 2.10-2.01 (m, 1H), 1.85-1.83 (m 2H), 1.83-1.72 (m, 3H), 1.51-1.44 (m, 4H).

Example 26 was synthesized according to the procedure described for the synthesis of N-(2-(2,6-dioxopiperidin-3-yl)-1,3-dioxoisoindolin-5-yl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide (Example 25) using appropriate building blocks and modified reaction conditions (such as reagents, reagent ratio, temperature, and reaction time) and purification conditions as needed.

| Example | Structure | Analytical Data |
|---|---|---|
| 26 | 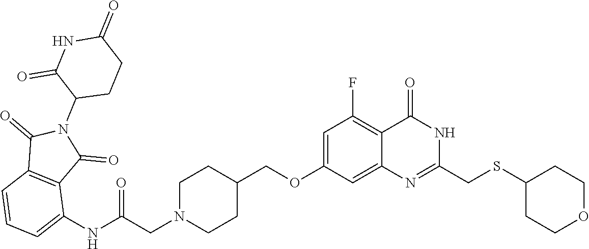 [SMILES: O=C1CCC(N2C(=O)c3cccc(NC(=O)CN4CCC(COc5cc(F)c6c(=O)[nH]c(CSC7CCOCC7)nc6c5)CC4)c3C2=O)C(=O)N1] N-(2-(2,6-dioxopiperidin-3-yl)-1,3- dioxoisoindolin-4-yl)-2-(4-(((5-fluoro-4-oxo- 2-(((tetrahydro-2H-pyran-4-yl)thio)methyl)- | LCMS (ESI, m/z): 721.25[M + H]⁺, ¹H NMR (400 MHz, DMSO-d₆) δ 12.13 (s, 1H), 11.14 (s, 1H), 11.05 (s, 1H), 8.79 (d, J = 8.4 Hz, 1H), 7.85 (dd, J = 8.5, 7.3 Hz, 1H), 7.60 (d, J = 7.2 Hz, 1H), 6.94(s, 1H), 6.88 (s, 2H), 5.14 − (dd, J = 12.8, 5.4 Hz, 1H), 4.02 (d, J = 6.2 Hz, 2H), 3.86 − 3.75 (m, 2H), 3.62 (s, 2H), 3.38-3.30 (m, 2H), 3.27-3.21 (m, 1H), 3.19 −3.11(m, 1H), 3.12-3.00 (m, 1H), 2.97-2.83 (m, 3H), 2.66-2.51 (m, 2H), 2.37-2.23 (m, 2H), 2.15-2.09 (m, 1H), 1.91-1.82 (m, 5H), 1.56-1.38 (m, 4H). |
| | 3,4-dihydroquinazolin-7- | |
| | yl)oxy)methyl)piperidin-1-yl)acetamide | |

### Example 27: Synthesis of 2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)isoindoline-1,3-dione

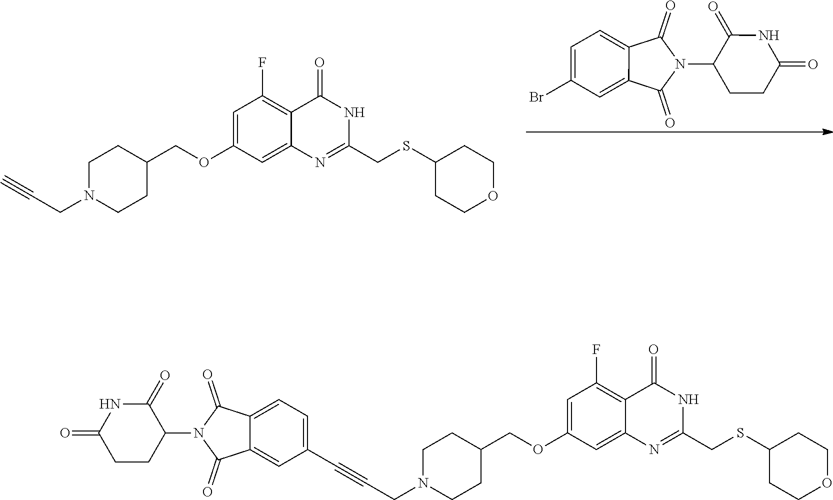 [SMILES: C#CCN1CCC(COc2cc(F)c3c(=O)[nH]c(CSC4CCOCC4)nc3c2)CC1 ; O=C1CCC(N2C(=O)c3ccc(Br)cc3C2=O)C(=O)N1 ; O=C1CCC(N2C(=O)c3ccc(C#CCN4CCC(COc5cc(F)c6c(=O)[nH]c(CSC7CCOCC7)nc6c5)CC4)cc3C2=O)C(=O)N1]

A solution of 5-fluoro-7-((1-(prop-2-yn-1-yl)piperidin-4-yl)methoxy)-2-(((tetrahydro-2H-pyran-4-yl)thio)methyl)quinazolin-4(3H)-one (70 mg, 0.157 mmol, 1.0 equiv), 5-bromo-2-(2,6-dioxopiperidin-3-yl)isoindole-1,3-dione (63.6 mg, 0.19 mmol, 1.2 equiv), TEA (47.7 mg, 0.47 mmol, 3.0 equiv) and Pd(PPh₃)₂Cl₂ (11.0 mg, 0.016 mmol, 0.1 equiv) in DMSO (2 mL) was stirred for 4 hours at 80° C. under a nitrogen atmosphere. The reaction was purified by reverse flash chromatography with the following conditions: column, C18 silica gel; mobile phase, MeCN in water (10 mmol/L NH₄HCO₃), 10% to 70% gradient in 30 min; detector, UV 254 nm to afford 2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)isoindoline-1,3-dione (47.5 mg, 43%) as a white solid. LCMS (ESI, m/z): 702.25 [M+H]⁺. ¹H NMR (300 MHz, DMSO-d₆) δ 11.52 (s, 2H), 8.01-7.77 (m, 3H), 6.88 (dd, J=11.0, 2.1 Hz, 2H), 5.17 (dd, J=12.9, 5.4 Hz, 1H), 4.00 (d, J=5.7 Hz, 2H), 3.88-3.75 (m, 2H), 3.61 (d, J=3.8 Hz, 4H), 3.36 (d, J=2.4 Hz, 1H), 3.29 (d, J=2.3 Hz, 1H), 3.13-2.98 (i, 1H), 2.92-2.81 (m, 3H), 2.67-2.52 (m, 2H), 2.31-2.23 (m, 2H), 2.11-2.02 (m, 1H), 1.87-1.65 (m, 5H), 1.55-1.29 (m, 4H).

Examples 28-33 were synthesized according to the procedure described for the synthesis of 2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-17-yl)prop-1-yn-1-yl)isoindoline-1,3-dione (Example 27) using appropriate building blocks and modified reaction conditions (such as reagents, reagent ratio, temperature, and reaction time) and purification conditions as needed.

| Example | Structure | Analytical Data |
|---|---|---|
| 28 | 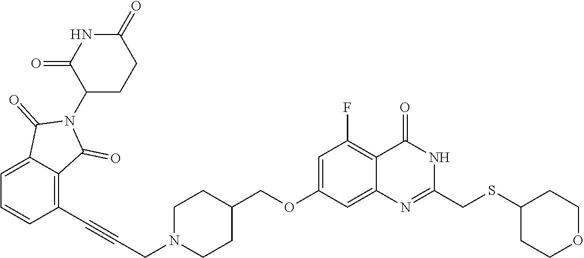 [SMILES: O=C1CCC(N2C(=O)c3cccc(C#CCN4CCC(COc5cc(F)c6c(=O)[nH]c(CSC7CCOCC7)nc6c5)CC4)c3C2=O)C(=O)N1] | LCMS (ESI, m/z): 702.30 [M + H]⁺. ¹H NMR (300 MHz, DMSO-d₆) δ 12.20 (s, ^{1H),} 11.13 (s, ^{1H),} 7.95 − 7.79 (m, 3H), 7.67 − 7.53 (m, ^{1H),} 6.91 (s, ^{1H),} 6.87 (s, ^{1H),} 5.16 (dd, J = 12.0, 6 Hz, ^{1H),} 4.05-3.90 (m, 2H), 3.89-3.75 (m, 2H), 3.74-3.63 (m, 3H), 3.36 − 3.29 (m, ^{1H),} 3.11-2.85(m, 4H), 2.63 − 2.55 (m, 2H), 2.39 − 2.33 (m, ^{1H),} 2.12 − 2.02 (m, 2H), 1.89 − 1.75 (m, 5H), 1.54 −1.25 (m, 4H). |
| | 2-(2,6-dioxopiperidin-3-yl)-4-(3-(4-(((5-fluoro-4- | |
| | oxo-2-(((tetrahydro-2H-pyran-4-yl)thio)methyl)- | |
| | 3,4-dihydroquinazolin-7-yl)oxy)methyl)piperidin- | |
| | 1-yl)prop-1-yn-1-yl)isoindoline-1,3-dione | |
| 29 | 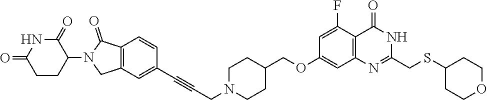 [SMILES: O=C1CCC(N2Cc3cc(C#CCN4CCC(COc5cc(F)c6c(=O)[nH]c(CSC7CCOCC7)nc6c5)CC4)ccc3C2=O)C(=O)N1] 3-(5-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)prop-1-yn-1-yl)-1- oxoisoindolin-2-yl)piperidine-2,6-dione | LCMS (ESI, m/z): 688.25 [M + H]⁺. ¹H NMR (400 MHz, DMSO-d₆) δ 11.03 (s, 2H), 7.77-7.67 (m, 2H), 7.65-7.50 (m, 2H), 6.88 (d, J = 10.4 Hz, 2H), 5.12 (dd, J = 13.3, 5.1 Hz, 1H), 4.46 (d, J = 17.5 Hz, 1H), 4.34 (d, J = 17.6 Hz, 1H), 4.02- 3.90 (m, 2H), 3.87-3.76 (m, 2H), 3.62 (s, 2H), 3.56 (s, 2H), 3.29-3.21 (m, 1H), |
| | | 3.12-3.00(m, 1H), 2.99 − |
| | | 2.83 (m, 3H), 2.63 (dd, J = |
| | | 4.7, 2.5 Hz, 1H), 2.47-2.35 |
| | | (m, 1H), 2.24 (t, J = 11.1 |
| | | Hz, 2H), 2.07-1.96 (m, |
| | | 1H), 1.88-1.70 (m, 5H), |
| | | 1.51-1.29 (m, 4H), 1.21- |
| | | 1.12 (m, 1H). |
| 30 | 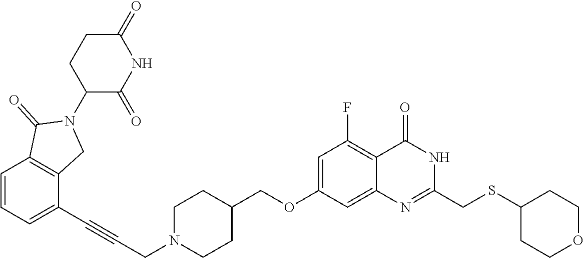 [SMILES: O=C1CCC(N2Cc3c(C#CCN4CCC(COc5cc(F)c6c(=O)[nH]c(CSC7CCOCC7)nc6c5)CC4)cccc3C2=O)C(=O)N1] 3-(4-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)prop-1-yn-1-yl)-1- oxoisoindolin-2-yl)piperidine-2,6-dione | LCMS (ESI, m/z): 688.30 [M + H]⁺. ¹H NMR (400 MHz, DMSO-d₆) δ 12.15 (s, 1H), 10.99 (s, 1H), 7.73 (dd, J = 16.0, 7.6, 1.1 Hz, 2H), 7.55 (t, J = 7.6 Hz, 1H), 6.88 (s, 1H), 6.87- 6.85 (m, 1H), 5.14 (dd, J = 13.3, 5.1 Hz, 1H), 4.49 (d, J = 17.7 Hz, 1H), 4.36 (d, J = 17.6 Hz, 1H), 4.00 (d, J = 5.8 Hz, 2H), 3.85-3.78 (m, 2H), 3.61 (d, J = 12.3 Hz, 4H), 3.37-3.27(m, 2H), 3.08-3.02 (m, 1H), 2.96- 2.85 (m, 3H), 2.65 − 2.56 (m, 1H), 2.49-2.39 (m, 1H), 2.25 (t, J = 11.3 Hz, 2H), 2.08-1.99 (m, 1H), 1.93-1.85 (m, 2H), 1.88- 1.81 (m, 3H), 1.49-1.33 |
| | | (m, 4H). |
| 31 | 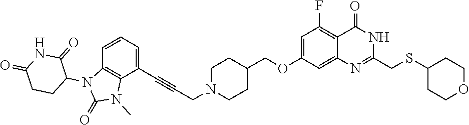 [SMILES: Cn1c(=O)n(C2CCC(=O)NC2=O)c2cccc(C#CCN3CCC(COc4cc(F)c5c(=O)[nH]c(CSC6CCOCC6)nc5c4)CC3)c21] 3-(4-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)prop-1-yn-1-yl)-3- methyl-2-oxo-2,3-dihydro-1H-benzo[d]imidazol- 1-yl)piperidine-2,6-dione | LCMS (ESI, m/z): 703.30 [M + H]⁺, ¹H NMR (400 MHz, DMSO-d₆) δ 12.05 (s, 1H), 11.09 (s, 1H), 7.22 − 7.06 (m, 2H), 7.02 (t, J = 7.9 Hz, 1H), 6.88 (d, J = 10.1 Hz, 2H), 5.39 (dd, J = 12.6, 5.4 Hz, ^{1H),} 4.00 (d, J = 5.9 Hz, 2H), 3.84-3.79 (m, 2H), 3.69-3.54 (m, 7H), 3.36 (d, J = 2.3 Hz, 2H), 3.12-3.00 (m, 1H), 3.01-2.81 (m, 3H), 2.73- 2.60 (m, 2H), 2.24 (t, J = |
| | | 11.3 Hz, 2H), 2.11-1.97 |
| | | (m, 1H), 1.89 (d, J = 13.0 |
| | | Hz, 2H), 1.81-1.72 (m, |
| | | 3H), 1.55-1.30 (m, 4H). |
| 32 | 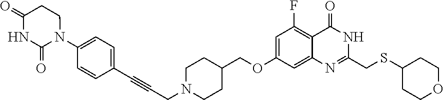 [SMILES: O=C1CCN(c2ccc(C#CCN3CCC(COc4cc(F)c5c(=O)[nH]c(CSC6CCOCC6)nc5c4)CC3)cc2)C(=O)N1] 1-(4-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)prop-1-yn-1- yl)phenyl)dihydropyrimidine-2,4(1H,3H)-dione | LCMS (ESI, m/z): 634.25 [M + H]⁺, ¹H NMR (400 MHz, DMSO-d₆) δ 12.08 (s, 1H), 10.42 (s, 1H), 7.45 (d, J = 8.0 Hz, 2H), 7.33 (d, J = 12.0 Hz, 2H), 6.92 − 6.84 (m, 2H), 4.00 (d, J = 5.8 Hz, 2H), 3.86 − 3.77 (m, 4H), 3.62 (s, 2H), 3.51 (s, 2H), 3.36-3.31 (m, ^{1H),} 3.30 (d, J = 4.0 Hz, 1H), 3.12-3.00 (m, 1H), 2.91 (d, |
| | | J = 10.7 Hz, 2H), 2.71 (t, J = |
| | | 6.6 Hz, 2H), 2.26-2.16 |
| | | (m, 2H), 1.89 (d, J = 13.0 |
| | | Hz, 2H), 1.80 (d, J = 12.6 |
| | | Hz, 3H), 1.52-1.29 (m, 4H). |
| 33 | 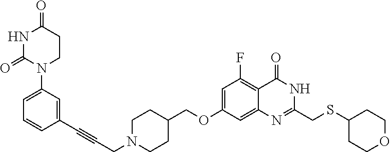 [SMILES: O=C1CCN(c2cccc(C#CCN3CCC(COc4cc(F)c5c(=O)[nH]c(CSC6CCOCC6)nc5c4)CC3)c2)C(=O)N1] 1-(3-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)prop-1-yn-1- yl)phenyl)dihydropyrimidine-2,4(1H,3H)-dione | LCMS (ESI, m/z): 634.20 [M + H]⁺, ¹H NMR (400 MHz, DMSO-d₆) δ 12.14 (s, 1H), 10.39 (s, 1H), 7.45-7.27 (m, 4H), 6.89 (s, 1H), 6.86 (s, 1H), 4.00 (d, J = 4.2 Hz, 2H), 3.85-3.75 (m, 4H), 3.62 (s, 2H), 3.52 (s, 2H), 3.38-3.25 (m, 3H), 3.14-3.00 (m, 1H)^{,} 2.91 (d, J = 10.8 Hz, 2H), 2.71 (t, J = 6.7 Hz, 2H), 2.29-2.15 (m, 2H), 1.89-1.70 (m, 4H), 1.51-1.21 (m, 4H). |

### Example 34: Synthesis of 2-(2,6-dioxopiperidin-3-yl)-5-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1l-yl)isoindoline-1,3-dione

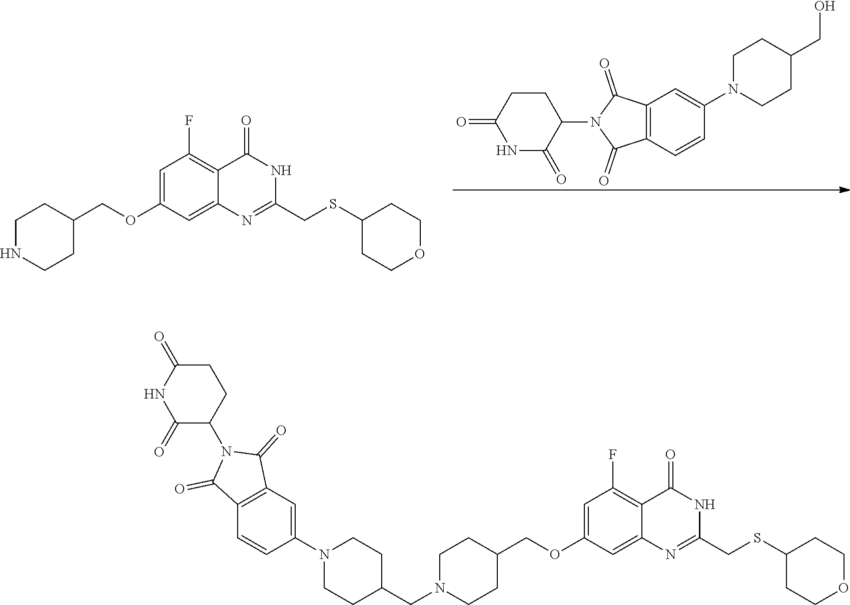 [SMILES: O=c1[nH]c(CSC2CCOCC2)nc2cc(OCC3CCNCC3)cc(F)c12 ; O=C1CCC(N2C(=O)c3ccc(N4CCC(CO)CC4)cc3C2=O)C(=O)N1 ; O=C1CCC(N2C(=O)c3ccc(N4CCC(CN5CCC(COc6cc(F)c7c(=O)[nH]c(CSC8CCOCC8)nc7c6)CC5)CC4)cc3C2=O)C(=O)N1]

A solution of 2-(2,6-dioxopiperidin-3-yl)-5-(4-(hydroxymethyl)piperidin-1-yl)isoindoline-1,3-dione (200 mg, 0.539 mmol, 1.0 equiv) and Dess-Martin reagent (251 mg, 0.593 mmol, 1.1 equiv) in DCM (4 mL) was stirred for 2 hours. 5-fluoro-7-(piperidin-4-ylmethoxy)-2-(((tetrahydro-2H-pyran-4-yl)thio)methyl)quinazolin-4(3H)-one (221 mg, 0.541 mmol, 1.0 equiv) was added dropwise over 1 min then stirred for 2 hours. STAB (102 mg, 2.71 mmol, 5.0 equiv) was added portion-wise and the resulting mixture stirred for 30 min. The resulting mixture was concentrated under reduced pressure and the residue purified by reverse phase flash chromatography with the following conditions: (column, C18 silica gel; mobile phase, MeCN in water (10 mmol/L NH₄HCO₃), 10% to 70% gradient in 15 min; detector, UV 254 nm) and further purified by Prep-HPLC with the following condition: Column: XBridge Shield RP18 OBD Column, 30*150 mm, 5 um; Mobile Phase A: Water (10 mmol/L NH₄HCO₃), Mobile Phase B: ACN; Flow rate: 60 mL/min; Gradient: 31% B to 55% B in 9 min, 55% B; Wave Length: 254/220 nm; RT (min): 9) to afford 2-(2,6-dioxopiperidin-3-yl)-5-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione (18.3 mg, 4%) as a yellow green solid. LCMS (ES, m/z): 761.60 [M+H]⁺. ¹H NMR (300 MHz, DMSO-d₆) δ 12.08 (s, 1H), 11.08 (s, 1H), 7.65 (d, J=8.5 Hz, 1H), 7.31 (s, 1H), 7.23 (d, J=9.4 Hz, 1H), 6.93-6.82 (m, 2H), 5.07 (dd, J=12.6, 5.3 Hz, 1H), 4.08-3.82 (m, 5H), 3.88-3.75 (m, 2H), 3.62 (s, 2H), 3.07-2.83 (m, 6H), 2.72-2.54 (m, 2H), 2.14 (d, J=6.5 Hz, 2H), 2.06-1.97 (m, 1H), 1.99-1.67 (m, 12H), 1.42-0.99 (m, 6H).

### Example 35: Synthesis of 2-(2,6-dioxopiperidin-3-yl)-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione

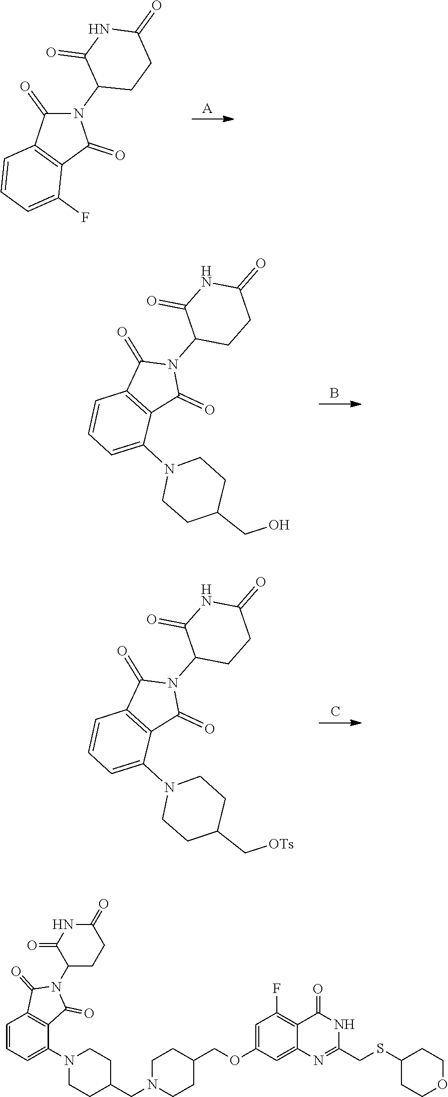

### Step A

A solution of 2-(2,6-dioxopiperidin-3-yl)-4-fluoroisoindole-1,3-dione (5.0 g, 18.1 mmol, 1.0 equiv) piperidin-4-ylmethanol (2.50 g, 21.7 mmol, 1.2 equiv) and DIEA (7.02 g, 54.3 mmol, 3.0 equiv) in NMP (100 mL) was stirred for 4 hours at 120° C. Water was added and the resulting mixture extracted with EtOAc (3×150 mL). The combined organic layers were washed with brine (3×200 mL), dried over anhydrous Na₂SO₄, filtered and concentrated under reduced pressure. The residue was purified by silica gel column chromatography, eluting with PE/EtOAc (2:3) to afford 2-(2,6-dioxopiperidin-3-yl)-4-(4-(hydroxymethyl)piperidin-1-yl)isoindoline-1,3-dione (6.7 g, 99%) as a yellow oil. LCMS (ESI, m/z): 372.15 [M+H]⁺.

### Step B

A solution of 2-(2,6-dioxopiperidin-3-yl)-4-(4-(hydroxymethyl)piperidin-1-yl)isoindoline-1,3-dione (500 mg, 1.35 mmol, 1.0 equiv), p-toluenesulfonyl chloride (308 mg, 1.62 mmol, 1.2 equiv), TEA (409 mg, 4.04 mmol, 3.0 equiv) and DMAP (82 mg, 0.673 mmol, 0.5 equiv) in DCM (40 mL) was stirred overnight. The resulting mixture was concentrated under vacuum. The residue was purified by silica gel column chromatography, eluted with PE/EA (3:1) to afford (1-(2-(2,6-dioxopiperidin-3-yl)-1,3-dioxoisoindolin-4-yl)piperidin-4-yl)methyl 4-methylbenzenesulfonate (620 mg, 88%) as a yellow solid. LCMS (ESI, m/z): 526.15 [M+H]⁺.

### Step C

A solution of {1-[2-(2,6-dioxopiperidin-3-yl)-1,3-dioxoisoindol-4-yl]piperidin-4-yl}methyl 4-methylbenzenesulfonate (100 mg, 0.19 mmol, 1.0 equiv), 5-fluoro-2-[(oxan-4-ylsulfanyl)methyl]-7-(piperidin-4-ylmethoxy)-3H-quinazolin-4-one (78 mg, 0.19 mmol, 1.0 equiv), DIEA (74 mg, 0.57 mmol, 3.0 equiv) and KI (15.8 mg, 0.095 mmol, 0.5 equiv) in ACN (5 mL) was stirred for 1 hour at 60° C. The resulting mixture was concentrated under vacuum and the residue was purified by reversed-phase flash chromatography with the following conditions: (column, C18 silica gel; mobile phase, MeCN in water (10 mmol/L NH₄HCO₃), 5% to 95% gradient in 20 min; detector, UV 254 nm) and further purified by prep-HPLC with the following conditions: Column: YMC-Actus Triart C18 ExRS, 30*150 mm, 5 μm; Flow rate: 60 mL/min; Gradient: 27% B to 47% B in 10 min, 47% B; Wave Length: 254/220 nm; RT (min): 10.92) to afford 2-(2,6-dioxopiperidin-3-yl)-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione (32 mg, 22%) as a yellow solid. LCMS (ESI, m/z): 761.35 [M+H]⁺. ¹H NMR (300 MHz, DMSO-d₆) δ 12.07 (s, 1H), 11.05 (s, 1H), 7.56 (q, J=7.8 Hz, 1H), 7.11 (dq, J=7.9, 4.2 Hz, 2H), 6.88 (dt, J=5.8, 2.7 Hz, 2H), 5.13-4.99 (m, 1H), 3.98 (s, 2H), 3.89-3.74 (m, 2H), 3.72-3.54 (m, 4H), 3.54-3.38 (m, 2H), 3.14-2.99 (m, 1H), 2.99-2.78 (m, 1H), 2.75-2.55 (m, 2H), 2.40-2.28 (m, 2H), 2.27-2.16 (m, 1) 2.16-2.06 (m, 1H), 2.06-1.96 (m, 1H) 1.95-1.81 (m, 4H), 1.81-1.66 (m, 3H), 1.66-1.53 (m, 3H), 1.53-1.38 (m, 3H), 1.38-1.15 (m, 3H).

Examples 36 and 37 were synthesized according to the procedure described for the synthesis 2-(2,6-dioxopiperidin-3-yl)-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione (Example 35) using appropriate building blocks and modified reaction conditions (such as reagents, reagent ratio, temperature, and reaction time) and purification conditions as needed.

| Example | Structure | Analytical Data |
|---|---|---|
| 36 | 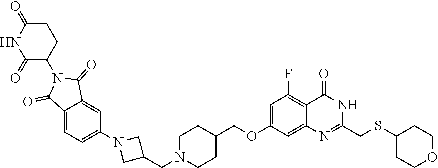 [SMILES: O=C1CCC(N2C(=O)c3ccc(N4CC(CN5CCC(COc6cc(F)c7c(=O)[nH]c(CSC8CCOCC8)nc7c6)CC5)C4)cc3C2=O)C(=O)N1] 2-(2,6-dioxopiperidin-3-yl)-5-(3-((4-(((5- fluoro-4-oxo-2-(((tetrahydro-2H-pyran-4- yl)thio)methyl)-3,4-dihydroquinazolin-7- yl)oxy)methyl)piperidin-1-yl)methyl)azetidin- 1-yl)isoindoline-1,3-dione | LCMS (ESI, m/z): 733.35 [M + H]⁺, ¹H NMR (400 MHz, DMSO-d₆) δ 12.16 (s, 1H), 11.07 (s, 1H), 7.63 (d, J = 8.3 Hz, 1H), 6.95 − 6.82 (m, 2H), 6.77 (d, J = 2.0 Hz, 1H), 6.64 (dd, J = 8.4, 2.1 Hz, 1H), 5.05 (dd, J = 12.8, 5.4 Hz, 1H), 4.13 (t, J = 8.2 Hz, 2H), 3.99 (d, J = 5.8 Hz, 2H), 3.82 (d, 3.7 Hz, 2H), 3.68 (dd, J = 8.4, 5.4 Hz, 2H), 3.62 (s, 2H), 3.30-3.24 (m, 2H), 3.14-2.94 (m, 3H), 2.95-2.79 (m, 3H), 2.64-2.55 (m, 3H), 2.05-1.89 (m, 5H), 1.81-1.68 (m, 3H), 1.50-1.40 (m, 2H), 1.39-1.22 |
| | | (m, 2H). |
| 37 | 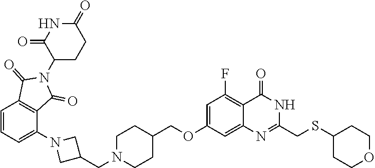 [SMILES: O=C1CCC(N2C(=O)c3cccc(N4CC(CN5CCC(COc6cc(F)c7c(=O)[nH]c(CSC8CCOCC8)nc7c6)CC5)C4)c3C2=O)C(=O)N1] 2-(2,6-dioxopiperidin-3-yl)-4-(3-((4-(((5- fluoro-4-oxo-2-(((tetrahydro-2H-pyran-4- yl)thio)methyl)-3,4-dihydroquinazolin-7- yl)oxy)methyl)piperidin-1-yl)methyl)azetidin- 1-yl)isoindoline-1,3-dione | LCMS (ESI, m/z): 733.30 [M + H]⁺. ¹H NMR (400 MHz, DMSO-d₆) δ 12.16 (s, 1H), 11.06 (s, 1H), 7.56 (t, J = 4.4 Hz, 1H), 7.10 (d, J = 7.2 Hz, 1H), 6.97-6.86 (m, 2H), 6.785 (d, J = 8.8 Hz, 1H), 5.05 (dd, J = 12.4, 5.2 Hz, 1H), 4.35-4.25 (m, 2H), 3.98(d, J = 5.6 Hz, 2H), 3.91-3.77 (m, 4H), 3.622(s, 2H), 3.40-3.35 (m, 1H), 3.10-3.00 (m, 1H), 2.96- 2.80 (m, 4H), 2.62-2.56 (m, 3H), 2.07-1.94 (m, 3H), 1.89 (d, J = 13.2 Hz, 2H), 1.75 (d, J = 10.0 Hz, 3H), 1.50-1.40 (m, 2H), 1.38 − 1.30 (m, 2H), |
| | | 1.35-1.24 (m, 1H). |

### Example 38: Synthesis of 3-(4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)-1-oxoisoindolin-2-yl)piperidine-2,6-dione

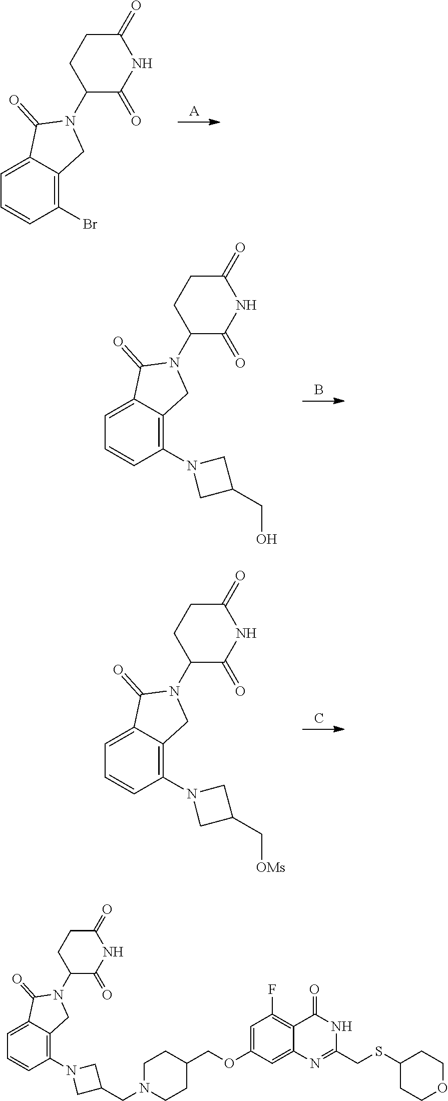

### Step A

A solution of 3-(4-bromo-1-oxoisoindolin-2-yl)piperidine-2,6-dione (160 mg, 0.49 mmol, 1.0 equiv), azetidin-3-yl methanol hydrochloride (73 mg, 0.59 mmol, 1.2 equiv), Pd-PEPPSI-IPentCl 2-methylpyridine (o-picoline) (42 mg, 0.05 mmol, 0.1 equiv) and Cs₂CO₃ (323 mg, 0.99 mmol, 2.0 equiv) in dioxane (10 mL) was stirred overnight at 100° C. under nitrogen atmosphere. The resulting mixture was filtered, the filter cake was washed with DCM (3×30 mL). The filtrate was concentrated under reduced pressure and the residue purified by reversed-phase flash chromatography with the following conditions: (column, C18 silica gel; mobile phase, MeCN in water, 5% to 20% gradient in 10 min; detector, UV 254 nm) to afford 3-(4-(3-(hydroxymethyl)azetidin-1-yl)-1-oxoisoindolin-2-yl)piperidine-2,6-dione (95 mg, 58%) as a white solid. LCMS (ESI, m/z): 330.15 [M+H]⁺.

### Step B

To a stirred solution of 3-(4-(3-(hydroxymethyl)azetidin-1-yl)-1-oxoisoindolin-2-yl)piperidine-2,6-dione (90 mg, 0.27 mmol, 1.0 equiv), TEA (138 mg, 1.37 mmol, 5.0 equiv) and DMAP (16.7 mg, 0.14 mmol, 0.5 equiv) in DCM (25 mL) was added methanesulfonic anhydride (143 mg, 0.82 mmol, 3.0 equiv) portion-wise at 0° C. then stirred for overnight at room temperature. The resulting mixture was concentrated under reduced pressure and the residue purified by reversed-phase flash chromatography with the following conditions: (column, C18 silica gel; mobile phase, MeCN in water, 5% to 30% gradient in 10 min; detector, UV 254 nm) to afford (1-(2-(2,6-dioxopiperidin-3-yl)-1-oxoisoindolin-4-yl)azetidin-3-yl)methyl methanesulfonate (78.2 mg, 70%) as a white solid. LCMS (ESI, m/z): 408.15 [M+H]⁺.

### Step C

A mixture of (1-(2-(2,6-dioxopiperidin-3-yl)-1-oxoisoindolin-4-yl)azetidin-3-yl)methyl methanesulfonate (78 mg, 0.19 mmol, 1.0 equiv), 5-fluoro-7-(piperidin-4-ylmethoxy)-2-(((tetrahydro-2H-pyran-4-yl)thio)methyl)quinazolin-4(3H)-one (86 mg, 0.21 mmol, 1.1 equiv), DIEA (223 mg, 1.73 mmol, 9.0 equiv) and KI (64 mg, 0.38 mmol, 2.0 equiv) in ACN (30 mL) was stirred overnight at 80° C. The resulting mixture was concentrated under reduced pressure and the residue was purified by C18 reverse phase chromatography eluting with water/ACN (63:37) and further purified by Prep-HPLC with the following condition (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 30% B to 40% B in 10 min, 40% B; Wave Length: 254 nm; RT (min): 8.6) to afford 3-(4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)-1-oxoisoindolin-2-yl)piperidine-2,6-dione (13.8 mg, 10%) as a white solid. LCMS (ESI, m/z): 719.35 [M+H]⁺. ¹H NMR (300 MHz, DMSO-d₆) δ 12.11 (s, 1H), 10.95 (s, 1H), 7.31 (t, J=7.7 Hz, 1H), 7.04 (d, J=7.4 Hz, 1H), 6.90-6.84 (m, 2H), 6.55 (d, J=7.9 Hz, 1H), 5.09 (dd, J=13.2, 5.1 Hz, 1H), 4.49-4.25 (m, 2H), 4.15-4.03 (m, 2H), 3.98 (d, J=5.8 Hz, 2H), 3.86-3.78 (m, 2H), 3.68-3.59 (m, 4H), 3.40-3.36 (m, 1H), 3.11-3.01 (m, 1H), 2.95-2.83 (m, 4H), 2.63-2.55 (m, 3H), 2.00-1.80 (m, 6H), 1.80-1.65 (m, 3H), 1.51-1.40 (m, 2H), 1.37-1.22 (m, 3H).

### Example 39: Synthesis of 2-(2,6-dioxopiperidin-3-yl)-4-(4-((1-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetyl)piperidin-4-yl)methyl)piperazin-1-yl)isoindoline-1,3-dione

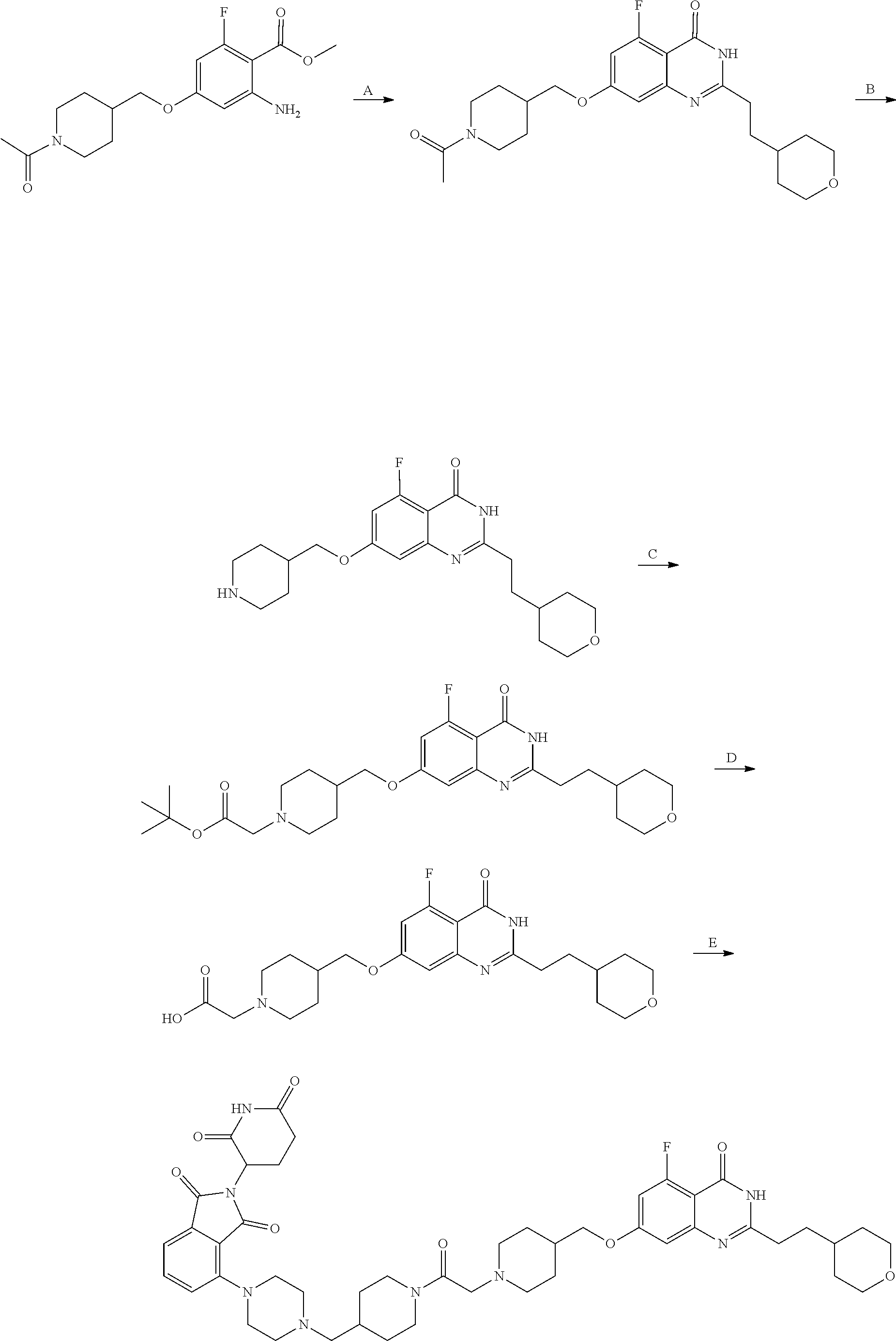

### Step A

A solution of methyl 4-[(1-acetylpiperidin-4-yl)methoxy]-2-amino-6-fluorobenzoate (2.0 g, 6.17 mmol, 1.0 equiv) and 3-(oxan-4-yl)propanenitrile (1.72 g, 12.3 mmol, 2.0 equiv) in HCl in 1,4-dioxane (50 mL, 4 M) was stirred overnight at 40° C. The mixture was neutralized to pH 7 with saturated aqueous NaHCO₃. The precipitated solids were collected by filtration and washed with water (3×10 mL) to afford 7-((1-acetylpiperidin-4-yl)methoxy)-5-fluoro-2-(2-(tetrahydro-2H-pyran-4-yl)ethyl)quinazolin-4(3H)-one (3.2 g) as a yellow crude solid that was used without further purification. LCMS (ESI, m/z): 432.20 [M+H]⁺.

### Step B

A solution of 7-((1-acetylpiperidin-4-yl)methoxy)-5-fluoro-2-(2-(tetrahydro-2H-pyran-4-yl)ethyl)quinazolin-4(3H)-one (3.2 g, 7.42 mmol, 1.0 equiv) and aqueous NaOH (20 ml, 5M) was stirred overnight at 80° C. The mixture was neutralized to pH 7 with concentrated HCl. The precipitated solids were collected by filtration and washed with water (3×10 mL) to afford 5-fluoro-7-(piperidin-4-ylmethoxy)-2-(2-(tetrahydro-2H-pyran-4-yl)ethyl)quinazolin-4(3H)-one (1.8 g, 62%) as a yellow solid. LCMS (ESI, m/z): 390.20 [M+H]⁺.

### Step C

A solution of 5-fluoro-7-(piperidin-4-ylmethoxy)-2-(2-(tetrahydro-2H-pyran-4-yl)ethyl)quinazolin-4(3H)-one (1.8 g, 4.62 mmol, 1.0 equiv), tert-butyl 2-bromoacetate (856 mg, 4.39 mmol, 0.95 equiv) and DIEA (1.79 g, 13.8 mmol, 3.0 equiv) in NMP (5 mL) was stirred overnight. The reaction mixture was purified by reversed-phase flash chromatography with the following conditions: (column, C18 silica gel; mobile phase, MeCN in water (10 mmol/L NH₄HCO₃), 5% to 95% gradient in 30 min; detector, UV 254 nm) to afford tert-butyl 2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetate (548 mg, 24%) as a white solid. LCMS (ESI, m/z): 504.30 [M+H]⁺.

### Step D

A solution of of tert-butyl 2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetate (548 mg, 1.09 mmol, 1.0 equiv) and TFA (2 mL) in DCM (10 mL) was stirred for 1 hour. The resulting mixture was concentrated under vacuum to afford 2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetic acid (1.3 g) as a red crude solid that was used without further purification. LCMS (ESI, m/z): 448.25 [M+H]⁺.

### Step E

A solution of 2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetic acid (196 mg, 0.45 mmol, 1.0 equiv), DIEA (173 mg, 1.34 mmol, 3.0 equiv) and HATU (204 mg, 0.54 mmol, 1.2 equiv) in DMF (3 mL) was stirred for 8 hours. The mixture was purified by reversed-phase flash chromatography with the following conditions: (column, C18 silica gel; mobile phase, MeCN in water (10 mmol/L NH₄HCO₃), 5% to 95% gradient in 25 min; detector, UV 254 nm) and further purified by Prep-HPLC with the following conditions (Column: Xselect CSH C18 OBD Column 30*150 mm 5 μm; Mobile Phase A: water (0.1% FA), Mobile Phase B: ACN; Flow rate: 60 mL/min; Gradient: 6% B to 20% B in 8 min, 20% B; Wave Length: 254/220 nm; RT (min): 8) to afford 2-(2,6-dioxopiperidin-3-yl)-4-(4-((1-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetyl)piperidin-4-yl)methyl)piperazin-1-yl)isoindoline-1,3-dione (16.5 mg, 4%) as a yellow solid. LCMS (ESI, m/z): 869.45 [M+H]⁺. ¹H NMR (300 MHz, Chloroform-d) δ 10.46 (s, 1H), 8.15 (s, 1H), 7.66-7.55 (m, 1H), 7.42 (d, J=7.1 Hz, 1H), 7.17 (d, J=8.4 Hz, 1H), 6.88 (s, 1H), 6.68 (dd, J=12.1, 2.3 Hz, 1H), 5.00-4.91 (m, 1H), 4.59 (d, J=13.2 Hz, 1H), 4.10 (d, J=12.6 Hz, 1H), 4.04-3.88 (m, 4H), 3.49-3.13 (m, 7H), 3.01-2.75 (m, 5H), 2.74-2.50 (m, 8H), 2.38-1.61 (m, 15H) 1.55-0.99 (m, 7H).

### Example 40: Synthesis of 2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)isoindoline-1,3-dione

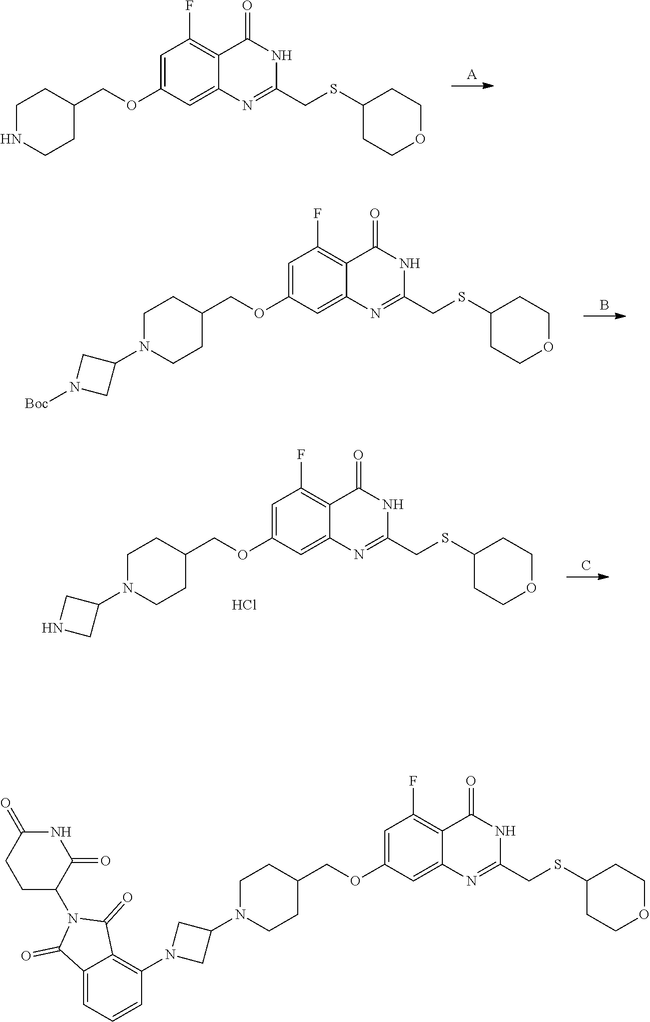

### Step A

A solution of 5-fluoro-7-(piperidin-4-ylmethoxy)-2-(((tetrahydro-2H-pyran-4-yl)thio)methyl)quinazolin-4(3H)-one (500 mg, 1.23 mmol, 1.0 equiv), tert-butyl 3-oxoazetidine-1-carboxylate (421 mg, 2.46 mmol, 2.0 equiv), HOAc (74 mg, 1.23 mmol, 1.0 equiv) and STAB (1303 mg, 6.15 mmol, 5.01 equiv) in DCE (10 mL) was stirred for 2 hours. The resulting mixture was concentrated under reduced pressure and the residue was purified by reverse flash chromatography with the following conditions: (column, C18 silica gel; mobile phase, MeCN in water (10 mmol/L NH₄HCO₃), 10% to 50% gradient in 15 min; detector, UV 254 nm) to afford tert-butyl 3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carboxylate. (468 mg, 68%) as a light yellow solid. LCMS (ESI, m/z): 563.45 [M+H]⁺.

### Step B

A solution of tert-butyl 3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carboxylate (200 mg, 0.36 mmol, 1.0 equiv) in HCl in 1,4-dioxane (10 mL, 1 mol/L) was stirred for 1 hour.

The resulting mixture was concentrated under reduced pressure to afford 7-((1-(azetidin-3-yl)piperidin-4-yl)methoxy)-5-fluoro-2-(((tetrahydro-2H-pyran-4-yl)thio)methyl)quinazolin-4(3H)-one hydrochloride that was used without further purification. LCMS (ESI, m/z): 463.35 [M+H]⁺.

### Step C

A solution of 7-((1-(azetidin-3-yl)piperidin-4-yl)methoxy)-5-fluoro-2-(((tetrahydro-2H-pyran-4-yl)thio)methyl)quinazolin-4(3H)-one hydrochloride (100 mg, 0.22 mmol, 1.0 equiv) and 2-(2,6-dioxopiperidin-3-yl)-4-fluoroisoindoline-1,3-dione (54 mg, 0.195 mmol, 0.90 equiv) in NMP (4 mL) was heated in a microwave for 1 hour at 120° C. The mixture was purified by reverse flash chromatography with the following conditions: (column, C18 silica gel; mobile phase, MeCN in water (10 mmol/L NH₄HCO₃), 10% to 50% gradient in 15 min; detector, UV 254 nm) and further purified by Prep-HPLC with the following condition:(Column: YMC-Actus Triart C18 ExRS, 30*150 mm, 5 μm; Mobile Phase A: water (10 mmol/L NH₄HCO₃), Mobile Phase B: ACN; Flow rate: 60 mL/min; Gradient: 33% B to 43% B in 10 min, 43% B; Wave Length: 254 nm; RT (min): 11.1) to afford 2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)isoindoline-1,3-dione (20.2 mg, 13%) as a yellow solid. LCMS (ESI, m/z): 719.35 [M+H]⁺. ¹H NMR (400 MHz, DMSO-d₆) δ 12.15 (s, 1H), 11.06 (s, 1H), 7.58 (t, J=7.6 Hz, 1H), 7.13 (d, J=6.8 Hz, 1H), 6.94-6.73 (m, 3H), 5.05 (dd, J=12.8, 5.6, 1H), 4.30 (t, J=6.8, 2H), 4.08-3.90 (m, 4H), 3.87-3.76 (m, 2H), 3.62 (s, 2H), 3.37-3.32 (m, 2H), 3.25-3.20 (m, 1H), 3.11-3.01 (m, 1H), 2.91-2.82 (m, 3H), 2.61-2.57 (m, 1H), 2.07-1.98 (m, 1H), 1.95-1.86 (m, 4H), 1.79-1.69 (m, 3H), 1.52-1.40 (m, 2H), 1.40-1.20 (m, 3H).

Examples 41 and 42 were synthesized according to the procedure described for the synthesis 2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)isoindoline-1,3-dione (Example 40) using appropriate building blocks and modified reaction conditions (such as reagents, reagent ratio, temperature, and reaction time) and purification conditions as needed.

| Example | Structure | Analytical Data |
|---|---|---|
| 41 | 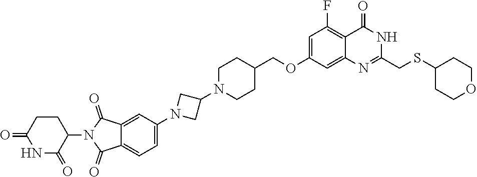 [SMILES: O=C1CCC(N2C(=O)c3ccc(N4CC(N5CCC(COc6cc(F)c7c(=O)[nH]c(CSC8CCOCC8)nc7c6)CC5)C4)cc3C2=O)C(=O)N1] 2-(2,6-dioxopiperidin-3-yl)-5-(3-(4-(((5-fluoro-4- oxo-2-(((tetrahydro-2H-pyran-4-yl)thio)methyl)- 3,4-dihydroquinazolin-7-yl)oxy)methyl)piperidin- 1-yl)azetidin-1-yl)isoindoline-1,3-dione | LCMS (ESI, m/z): 719.30 [M + H]⁺. ¹H NMR (400 MHz, DMSO-d₆) δ 12.15 (s, 1H), 11.07 (s, 1H), 7.66 (d, J = 8.3 Hz, 1H), 6.88 (dd, J = 10.5, 2.3 Hz, 2H), 6.80 (d, J = 2.1 Hz, 1H), 6.66 (dd, J = 8.3, 2.1 Hz, 1H), 5.06 (dd, J = 12.9, 5.4 Hz, 1H), 4.12 (t, J = 7.7 Hz, 2H), 4.00 (d, J = 5.9 Hz, 2H), 3.88 − 3.77 (m, 4H), 3.62 (s, 2H), 3.37- 3.26 (m, 2H), 3.11-3.00 (m, 1H), 2.96-2.80 (m, 3H), 2.71-2.53 (m, 2H), 2.08- |
| | | 1.97 (m, 1H), 1.95-1.84 (m, |
| | | 4H), 1.84-1.69 (m, 3H), |
| | | 1.53-1.22 (m, 4H). |
| 42 | 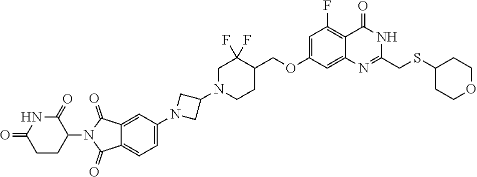 [SMILES: O=C1CCC(N2C(=O)c3ccc(N4CC(N5CCC(COc6cc(F)c7c(=O)[nH]c(CSC8CCOCC8)nc7c6)C(F)(F)C5)C4)cc3C2=O)C(=O)N1] 5-(3-(3,3-difluoro-4-(((5-fluoro-4-oxo-2- (((tetrahydro-2H-pyran-4-yl)thio)methyl)-3,4- dihydroquinazolin-7-yl)oxy)methyl)piperidin-1- yl)azetidin-1-yl)-2-(2,6-dioxopiperidin-3- yl)isoindoline-1,3-dione | LCMS (ESI, m/z): 755.30 [M + H]⁺. ¹H NMR (400 MHz, DMSO-d₆) δ 12.15 (s, 1H), 11.07 (s, 1H), 7.67 (d, J = 8.3 Hz, 1H), 6.92 (d, J = 11.7 Hz, 2H), 6.80 (d, J = 2.1 Hz, 1H), 6.67 (dd, J = 8.3, 2.1 Hz, 1H), 5.06 (dd, J = 12.9, 5.4 Hz, 1H), 4.40 (dd, J = 10.5, 5.3 Hz, 1H), 4.24-4.04 (m, 3H), 4.00- 3.71 (m, 5H), 3.63 (s, 2H), 3.59-3.51 (m, 1H), 3.14- 3.02 (m, 2H), 2.96 − 2.80 (m, 2H), 2.72-2.54 (m, 2H), 2.47-2.28 (m, 2H), 2.18-2.02 (m, 1H), 2.10- 1.94 (m, 2H), 1.94-1.84 (m, |
| | | 2H), 1.66-1.52 (m, 1H), |
| | | 1.51-1.38 (m, 3H). |

### Example 43: Synthesis of (E)-2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-en-1-yl)isoindoline-1,3-dione

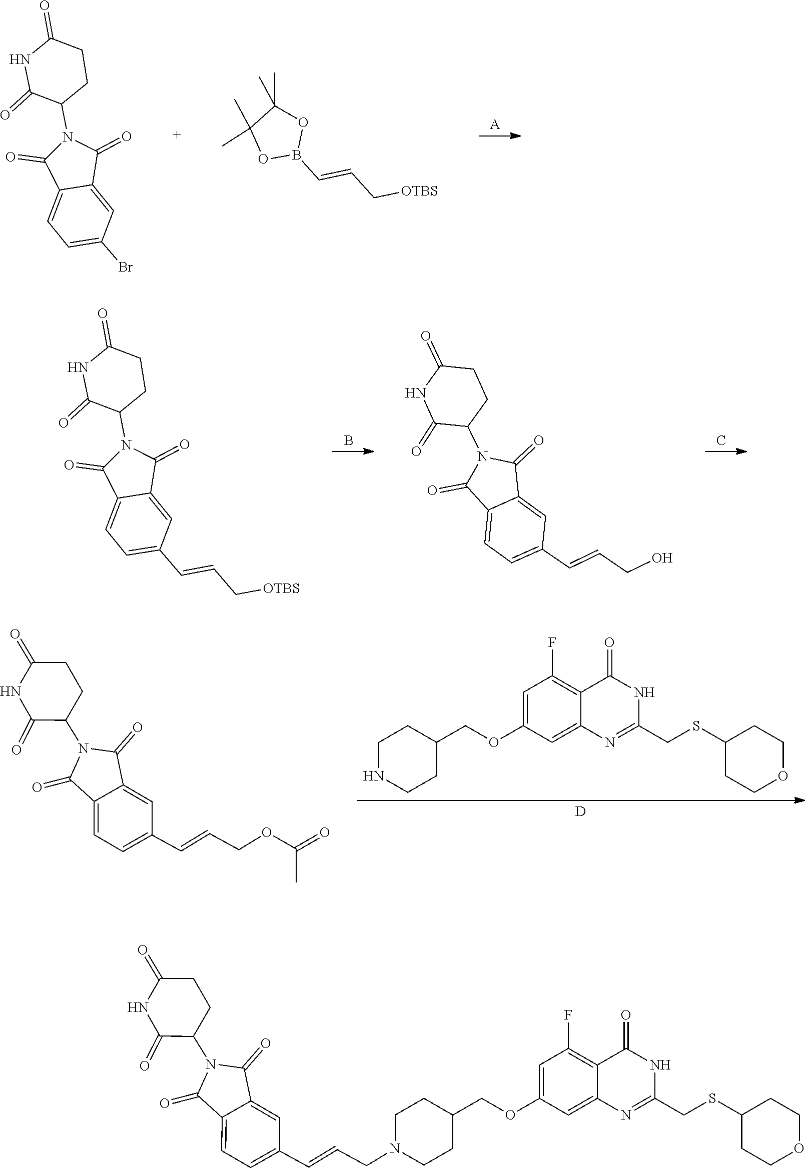

### Step A

A solution of 5-bromo-2-(2,6-dioxopiperidin-3-yl)isoindole-1,3-dione (500 mg, 1.48 mmol, 1.0 equiv), tert-butyldimethyl{[(2E)-3-(4,4,5,5-tetramethyl-1,3,2-dioxaborolan-2-yl)prop-2-en-1-yl]oxy}silane (442 mg, 1.48 mmol, 1.0 equiv), Pd(dppf)Cl₂ (217 mg, 0.297 mmol, 0.2 equiv) and NaHCO₃ (374 mg, 4.45 mmol, 3.0 equiv) in 1,4-dioxane (5 mL) and water (1 mL) was stirred for 1 hour at 90° C. under nitrogen atmosphere. The mixture was purified by reverse flash chromatography with the following conditions: column, C18 silica gel; mobile phase, MeCN in water (10 mmol/L NH₄HCO₃), 0% to 71% gradient in 10 min; detector, UV 254 nm to afford (E)-5-(3-((tert-butyldimethylsilyl)oxy)prop-1-en-1-yl)-2-(2,6-dioxopiperidin-3-yl)isoindoline-1,3-dione (330 mg, 52%) as a white solid. LCMS (ESI, m/z): 429.18 [M+H]⁺.

### Step B

A solution of 5-[(1E)-3-[(tert-butyldimethylsilyl)oxy]prop-1-en-1-yl]-2-(2,6-dioxopiperidin-3-yl)isoindole-1,3-dione (240 mg, 0.56 mmol, 1.0 equiv) and TBAF (293 mg, 1.12 mmol, 2.0 equiv) in THF (2 mL) was stirred for 1 hour. The mixture was purified by reverse flash chromatography with the following conditions: (column, C18 silica gel; mobile phase, MeCN in water (10 mmol/L NH₄HCO₃), 0% to 25% gradient in 10 min; detector, UV 254 nm) to afford (E)-2-(2,6-dioxopiperidin-3-yl)-5-(3-hydroxyprop-1-en-1-yl)isoindoline-1,3-dione (150 mg, 85%) as a white solid. LCMS (ESI, m/z): 315.09 [M+H]⁺.

### Step C

A solution of (E)-2-(2,6-dioxopiperidin-3-yl)-5-(3-hydroxyprop-1-en-1-yl)isoindoline-1,3-dione (90 mg, 0.29 mmol, 1.0 equiv), TEA (58 mg, 0.57 mmol, 2.0 equiv), Ac₂O (35.1 mg, 0.34 mmol, 1.2 equiv) and DMAP (3.50 mg, 0.029 mmol, 0.1 equiv) in THF (2 mL) was stirred for 1 hour. The mixture was purified by reverse flash chromatography with the following conditions: column, C18 silica gel; mobile phase, MeCN in water (10 mmol/L NH₄HCO₃), 0% to 48% gradient in 10 min; detector, UV 254 nm to afford (E)-3-(2-(2,6-dioxopiperidin-3-yl)-1,3-dioxoisoindolin-5-yl)allyl acetate (80 mg, 78%) as a white solid. LCMS (ESI, m/z): 357.10 [M+H]⁺.

### Step D

A solution of (E)-3-(2-(2,6-dioxopiperidin-3-yl)-1,3-dioxoisoindolin-5-yl)allyl acetat (30 mg, 0.095 mmol, 1.0 equiv), 5-fluoro-2-[(oxan-4-ylsulfanyl)methyl]-7-(piperidin-4-ylmethoxy)-3H-quinazolin-4-one (38.9 mg, 0.095 mmol, 1.0 equiv) and Pd(PPh₃)₄ (33.1 mg, 0.028 mmol, 0.3 equiv) in DCM (1.5 mL) was stirred for 1 hour at 40° C. under nitrogen atmosphere. The mixture was purified by reverse flash chromatography with the following conditions: (column, C18 silica gel; mobile phase, MeCN in water (10 mmol/L NH₄HCO₃), 0% to 49% gradient in 10 min; detector, UV 254 nm) and further purified by prep-HPLC with the following conditions (Column: YMC-Actus Triart C18 ExRS, 30*150 mm, 5 μm; Mobile Phase A: water (10 mmol/L NH₄HCO₃), Mobile Phase B: ACN; Flow rate: 60 mL/min; Gradient: 17% B to 42% B in 9 min, 42% B; Wave Length: 254/220 nm; RT (min): 8.9) to afford (E)-2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-en-1-yl)isoindoline-1,3-dione (12.2 mg, 17%) as a white solid. LCMS (ESI, m/z): 704.25 [M+H]⁺, ¹H NMR (300 MHz, DMSO-d₆) δ 12.15 (s, 1H), 11.11 (s, 1H), 8.01 (s, 1H), 7.96-7.88 (m, 1H), 7.84 (d, J=7.8 Hz, 1H), 6.93-6.82 (m, 2H), 6.79-6.62 (m, 2H), 5.14 (m, 1H), 3.98 (d, J=5.7 Hz, 2H), 3.85-3.75 (m, 2H), 3.60 (s, 2H), 3.28-3.21 (m, 2H), 3.15 (d, J=5.5 Hz, 2H), 3.11-2.98 (m, 1H), 2.96-2.89 (M, 3H), 2.59-2.52 (m, 1H), 2.05-1.94 (m, 3H), 1.87-1.81 (m, 2H), 1.80-1.70 (m, 3H), 1.53-1.28 (m, 5H).

### Example 44: Synthesis of 2-(2,6-dioxopiperidin-3-yl)-5-(4-(((5-fluoro-2-(((1-methylpiperidin-4-yl)thio)methyl)-4-oxo-3,4-dihydroquinazolin-7-yl)oxy)methyl)piperidin-1-yl)isoindoline-1,3-dione

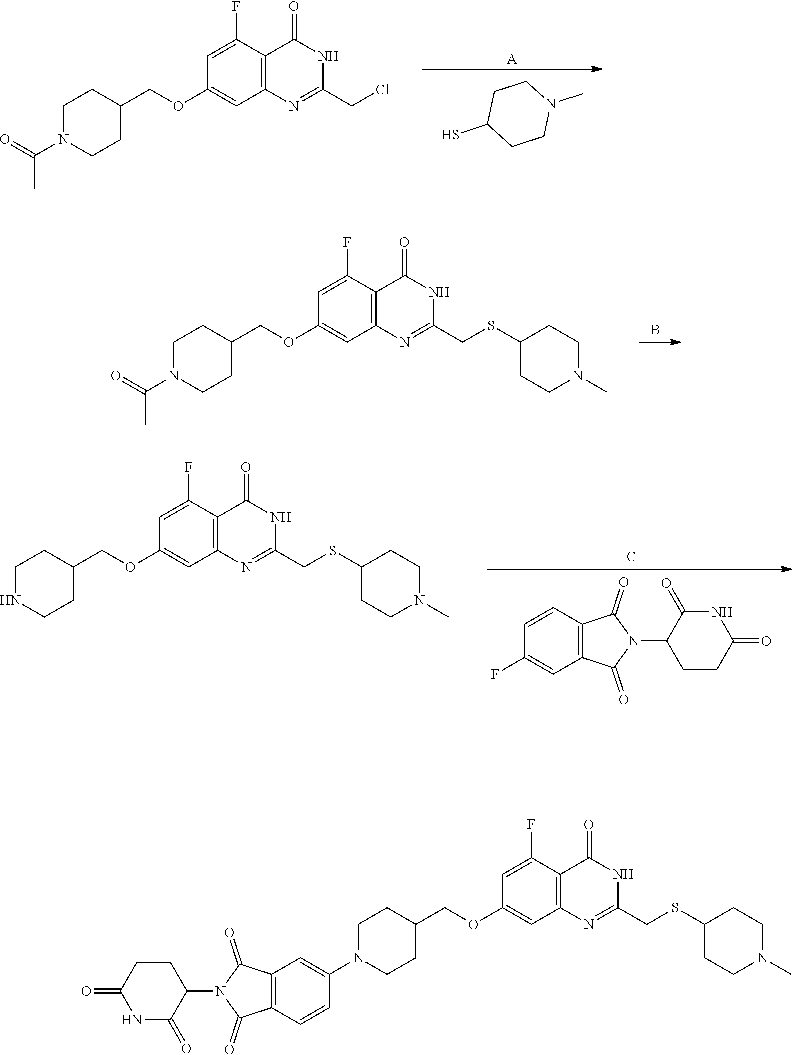

### Step A

A solution of 7-((1-acetylpiperidin-4-yl)methoxy)-2-(chloromethyl)-5-fluoroquinazolin-4(3H)-one (500 mg, 1.41 mmol, 1.0 equiv), 1-methylpiperidine-4-thiol (185 mg, 1.41 mmol, 1.0 equiv) and NaOH (226 mg, 5.65 mmol, 4.0 equiv) in water (3 mL) was stirred for 5 hours. The mixture was neutralized to pH 7 with HCl (12M). The residue was purified by reverse flash chromatography with the following conditions: column, C18 silica gel; mobile phase, MeCN in water (10 mmol/L NH₄HCO₃), 10% to 70% gradient in 15 min; detector, UV 254 nm to afford 7-((1-acetylpiperidin-4-yl)methoxy)-5-fluoro-2-(((1-methylpiperidin-4-yl)thio)methyl)quinazolin-4(3H)-one (526 mg, 80%) as a yellow solid. LCMS (ESI, m/z): 463.21 [M+H]⁺.

### Step B

A solution of 7-((1-acetylpiperidin-4-yl)methoxy)-5-fluoro-2-(((1-methylpiperidin-4-yl)thio)methyl)quinazolin-4(3H)-one (200 mg, 0.432 mmol, 1 equiv), HCl (1 mL) and water (2 mL) was stirred for 2 days at 70° C. The resulting mixture was concentrated under reduced pressure to afford 5-fluoro-2-(((1-methylpiperidin-4-yl)thio)methyl)-7-(piperidin-4-ylmethoxy)quinazolin-4(3H)-one (350 mg, crude) as a yellow oil that was used without further purification. LCMS (ESI, m/z): 421.20 [M+H]⁺.

### Step C

A solution of 5-fluoro-2-(((1-methylpiperidin-4-yl)thio)methyl)-7-(piperidin-4-ylmethoxy)quinazolin-4(3H)-one (50 mg, 0.119 mmol, 1.0 equiv), 2-(2,6-dioxopiperidin-3-yl)-5-fluoroisoindoline-1,3-dione (49.3 mg, 0.178 mmol, 1.5 equiv), DIEA (92.2 mg, 0.714 mmol, 6.0 equiv) in NMP (2 mL) was stirred for 12 hours at 120° C. The mixture was purified by reverse phase column and further purified by Prep-HPLC with the following conditions (Column: XBridge Shield RP18 OBD Column, 30*150 mm, 5 μm; Mobile Phase A: water (10 mmol/L NH₄HCO₃), Mobile Phase B: ACN; Flow rate: 60 mL/min; Gradient: 30% B to 40% B in 10 min, 40% B; Wave Length: 254 nm; RT (min): 8.5) to afford 2-(2,6-dioxopiperidin-3-yl)-5-(4-(((5-fluoro-2-(((1-methylpiperidin-4-yl)thio)methyl)-4-oxo-3,4-dihydroquinazolin-7-yl)oxy)methyl)piperidin-1-yl)isoindoline-1,3-dione (9.7 mg, 12%) as a yellow green solid. LCMS (ESI, m/z): 677.25 [M+H]⁺. ¹H NMR (400 MHz, DMSO-d₆) δ 11.08 (s, 1H), 7.67 (d, J=8.6 Hz, 1H), 7.35 (d, J=2.2 Hz, 1H), 7.27 (dd, J=8.6, 2.3 Hz, 1H), 6.89 (dd, J=11.0, 2.1 Hz, 2H), 5.07 (dd, J=12.9, 5.4 Hz, 1H), 4.15-4.09 (m, 2H), 4.05-4.01 (m, 2H), 3.62 (s, 2H), 3.08-3.00 (m, 2H), 2.93-2.86 (m, 1H), 2.82-2.74 (m, 1H), 2.69-2.61 (m, 3H), 2.58-2.55 (m, 1H), 2.13-2.10 (m, 4H), 1.96-1.83 (m, 7H), 1.50-1.33 (m, 5H).

### Example 45: Synthesis of 3-(4-((4-(4-(((5-fluoro-4-oxo-2-(((tetrahydro-2H-pyran-4-yl)thio)methyl)-3,4-dihydroquinazolin-7-yl)oxy)methyl)piperidin-1-yl)but-2-yn-1-yl)amino)phenyl)piperidine-2,6-dione

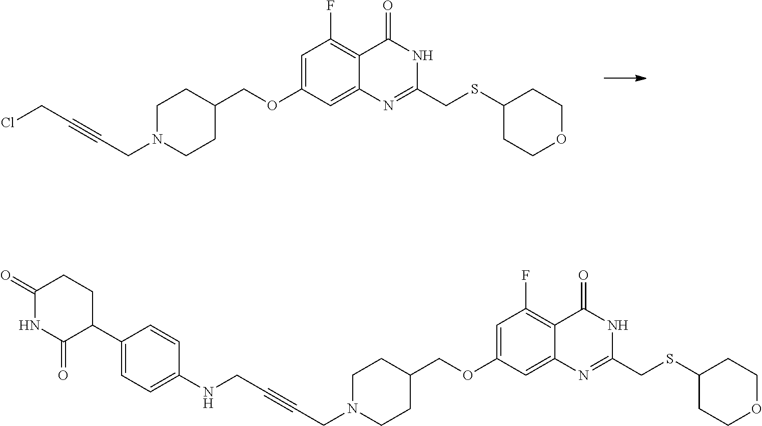 [SMILES: O=c1[nH]c(CSC2CCOCC2)nc2cc(OCC3CCN(CC#CCCl)CC3)cc(F)c12 ; O=C1CCC(c2ccc(NCC#CCN3CCC(COc4cc(F)c5c(=O)[nH]c(CSC6CCOCC6)nc5c4)CC3)cc2)C(=O)N1]

A solution of 3-(4-aminophenyl)piperidine-2,6-dione (100 mg, 0.49 mmol, 1.0 equiv) and 7-{[1-(4-chlorobut-2-yn-1-yl)piperidin-4-yl]methoxy}-5-fluoro-2-[(oxan-4-ylsulfanyl)methyl]-3H-quinazolin-4-one (242 mg, 0.49 mmol, 1.0 equiv) in EtOH (2.5 mL) was stirred for 1 day at 60° C. After concentration, the residue was purified by reversed-phase flash chromatography with the following conditions: column, C18 silica gel; mobile phase, MeCN in water (0.1% FA), 0% to 17% gradient in 20 min; detector, UV 254 nm. The crude product was further purified by prep-HPLC with the following conditions (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 27% B to 49% B in 8 min, 49% B; Wave Length: 254/220 nm; RT (min): 8) to afford 3-(4-((4-(4-(((5-fluoro-4-oxo-2-(((tetrahydro-2H-pyran-4-yl)thio)methyl)-3,4-dihydroquinazolin-7-yl)oxy)methyl)piperidin-1-yl)but-2-yn-1-yl)amino)phenyl)piperidine-2,6-dione (20.8 mg, 6%) as a white solid. LCMS (ESI, m/z): 662.35 [M+H]⁺, ¹H NMR (300 MHz, DMSO-d₆) δ 12.16 (s, 1H), 10.73 (s, 1H), 6.93 (d, J=8.3 Hz, 2H), 6.90-6.81 (m, 2H), 6.59 (d, J=8.4 Hz, 2H), 5.89 (t, J=6.2 Hz, 1H), 3.94-3.78 (m, 6H), 3.69-3.60 (m, 1H), 3.60 (s, 2H), 3.26-3.20 (m, 1H), 3.19 (s, 2H), 3.10-2.97 (m, 1H), 2.81-2.70 (m, 2H), 2.67-2.52 (m, 1H), 2.46-2.34 (m, 2H), 2.06 (s, 2H), 2.03-1.92 (m, 3H), 1.90-1.80 (m, 2H), 1.73-1.55 (m, 3H), 1.53-1.27 (m, 2H), 1.25-1.16 (m, 2H).

### Example 46: Synthesis of 3-((4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutyl)methoxy)phenyl)amino)piperidine-2,6-dione

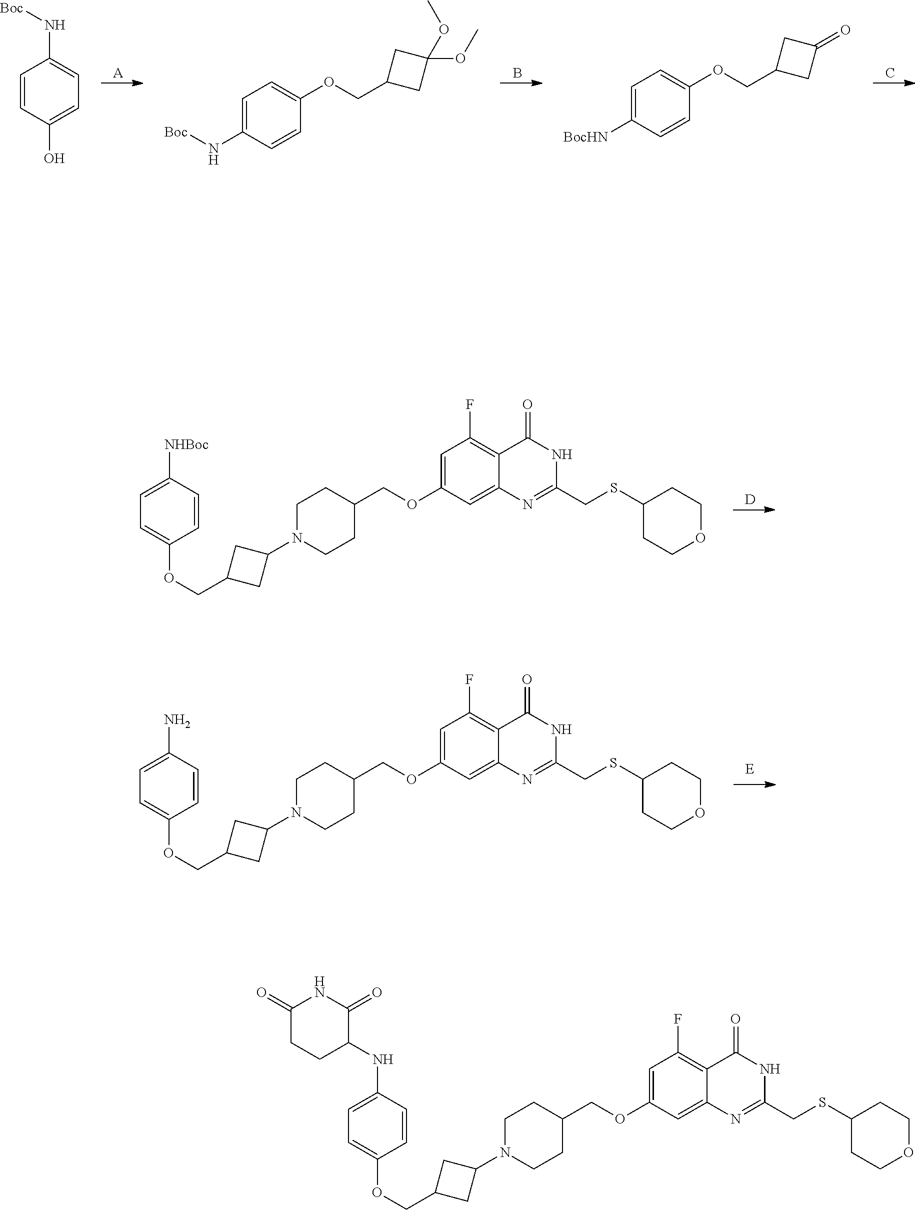

### Step A

A solution of tert-butyl N-(4-hydroxyphenyl)carbamate (1.0 g, 4.78 mmol, 1.0 equiv), (3,3-dimethoxycyclobutyl)methanol (1.05 g, 7.17 mmol, 1.5 equiv) and PPh₃ (1.88 g, 7.17 mmol, 1.5 equiv) in THF (10 mL) was stirred for 5 min at 0° C. To the mixture was added DEAD (1.25 g, 7.17 mmol, 1.5 equiv) dropwise at 0° C. The resulting mixture was stirred overnight at room temperature. After concentration, the residue was purified by silica gel column chromatography, eluting with PE/EtOAc (3:1) to afford tert-butyl (4-((3,3-dimethoxycyclobutyl)methoxy)phenyl)carbamate (1 g, 62%) as a off-white solid. LCMS (ESI, m/z): 338.20[M+H]⁺.

### Step B

A solution of tert-butyl (4-((3,3-dimethoxycyclobutyl)methoxy)phenyl)carbamate (570 mg, 1.69 mmol, 1.0 equiv) in THF (10 mL) was stirred for 2 min then HCl (8.45 mL, 8.45 mmol, 5.0 equiv) was added at 0° C. The resulting mixture was stirred for 5 hours at room temperature to afford tert-butyl (4-((3-oxocyclobutyl)methoxy)phenyl)carbamate (480 mg, 98%) as a off-white crude solid. The crude product was used in the next step directly without further purification. This resulted in LCMS (ESI, m/z): 292.15 [M+H]⁺.

### Step C

A solution of tert-butyl (4-((3-oxocyclobutyl)methoxy)phenyl)carbamate (328 mg, 1.13 mmol, 1.0 equiv), 5-fluoro-2-[(oxan-4-ylsulfanyl)methyl]-7-(piperidin-4-ylmethoxy)-3H-quinazolin-4-one (459 mg, 1.13 mmol, 1.0 equiv), NaBH(OAc)₃ (716 mg, 3.38 mmol, 3.0 equiv) and HOAc (67.6 mg, 1.13 mmol, 1.0 equiv) in THF (10 mL) was stirred overnight. After concentration, the residue was purified by reversed-phase flash chromatography with the following conditions: column, C18 silica gel; mobile phase, MeCN in water (0.1% FA), 10% to 68% gradient in 25 min; detector, UV 254 nm. This resulted in tert-butyl (4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutyl)methoxy)phenyl)carbamate (300 mg, 39%) as a white solid. LCMS (ESI, m/z): 683.50[M+H]⁺.

### Step D

A solution of of tert-butyl (4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutyl)methoxy)phenyl)carbamate (300 mg, 0.439 mmol, 1.0 equiv) in DCM (5 mL) and TFA (5 mL) was stirred for 1h. After concentration, the residue was purified by reversed-phase flash chromatography with the following conditions: column, C18 silica gel; mobile phase, MeCN in water (0.1% FA), 10% to 69% gradient in 20 min; detector, UV 254 nm. This resulted in 7-((1-(3-((4-aminophenoxy)methyl)cyclobutyl)piperidin-4-yl)methoxy)-5-fluoro-2-(((tetrahydro-2H-pyran-4-yl)thio)methyl)quinazolin-4(3H)-one (100 mg, 39%) as a off-white solid. LCMS (ESI, m/z): 583.30[M+H]⁺.

### Step E

A solution of 7-((1-(3-((4-aminophenoxy)methyl)cyclobutyl)piperidin-4-yl)methoxy)-5-fluoro-2-(((tetrahydro-2H-pyran-4-yl)thio)methyl)quinazolin-4(3H)-one (100 mg, 0.17 mmol, 1.0 equiv), 3-bromopiperidine-2,6-dione (39.5 mg, 0.21 mmol, 1.2 equiv) and NaHCO₃ (1.44 mg, 0.018 mmol, 2.0 equiv) in ACN (0.5 mL) was stirred for overnight at 80° C. The residue was purified by reversed-phase flash chromatography with the following conditions: column, C18 silica gel; mobile phase, MeCN in water (10 mmol/L NH₄HCO₃), 10% to 38% gradient in 20 min; detector, UV 254 nm. This resulted in 3-((4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutyl)methoxy)phenyl)amino)piperidine-2,6-dione (41.1 mg, 31%) as a white solid. LCMS (ESI, m/z): 684.25 [M+H]⁺. ¹H NMR (300 MHz, DMSO-d₆) δ 12.08 (s, 1H), 10.76 (s, 1H), 6.92-6.81 (m, 2H), 6.76-6.55 (m, 4H), 5.42 (d, J=7.3 Hz, 1H), 4.25-4.11 (m, 1H), 3.95 (d, J=5.6 Hz, 2H), 3.91-3.73 (m, 4H), 3.60 (s, 2H), 3.38-3.24 (m, 2H), 3.11-2.96 (m, 1H), 2.80-2.70 (m, 3H), 2.56-2.50 (m, 1H), 2.30-2.22 (m, 1H), 2.21-2.00 (m, 3H), 1.85-1.35 (m, 13H), 1.34-1.18 (m, 2H).

### Example 47: Synthesis of 3-({4-[3-({4-[({5-fluoro-2-[(oxan-4-ylsulfanyl)methyl]-4-oxo-3H-quinazolin-7-yl}oxy)methyl]piperidin-1-yl}methyl)cyclobutoxy]phenyl}amino)piperidine-2,6-dione

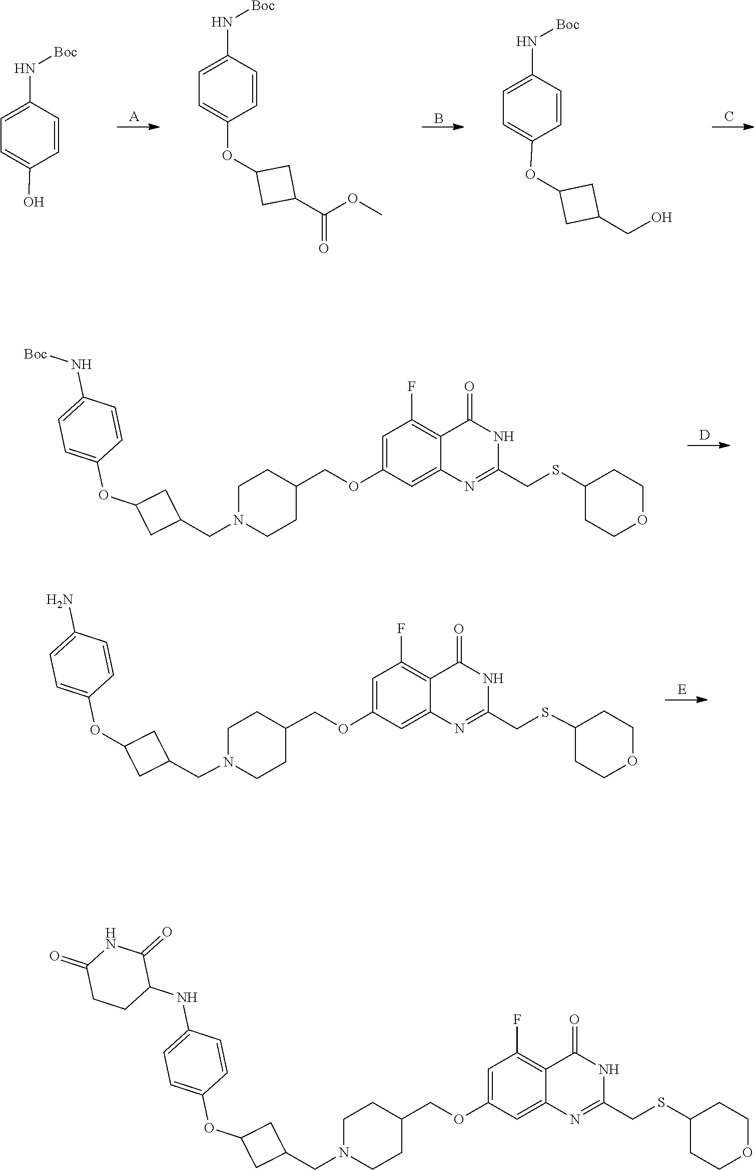

### Step A

A solution of tert-butyl N-(4-hydroxyphenyl)carbamate (1.0 g, 4.78 mmol, 1.0 equiv), methyl 3-hydroxycyclobutane-1-carboxylate (0.95 g, 7.31 mmol, 1.5 equiv), (1.05 g, 7.17 mmol, 1.5 equiv), DEAD (1.25 g, 7.17 mmol, 1.5 equiv) and PPh₃ (1.91 g, 7.26 mmol, 1.5 equiv) in THF (15 mL) was stirred for 2 hours at 50° C. The resulting mixture was concentrated under vacuum.

The residue was purified by silica gel column chromatography, eluting with PE/EtOAc (5:1) to afford methyl 3-{4-[(tert-butoxycarbonyl)amino]phenoxy}cyclobutane-1-carboxylate (1 g, 65%) as a white solid. LCMS (ESI, m/z): 322.35 [M+H]⁺.

### Step B

To a solution of methyl 3-{4-[(tert-butoxycarbonyl)amino]phenoxy}cyclobutane-1-carboxylate (980 mg, 3.05 mmol, 1.0 equiv) in THF (30 mL) was added LiAlH₄ (3.8 mL, 7.60 mmol, 2.49 equiv) at 0° C. then stirred for 1 hour at room temperature. The reaction was quenched with aqueous Na₂SO₄·10H₂O at 0° C. The resulting mixture was filtered and the filter cake was washed with DCM. The filtrate was concentrated under vacuum and the residue was purified by silica gel column chromatography, eluting with PE/EtOAc (2:1) to afford tert-butyl N-{4-[3-(hydroxymethyl)cyclobutoxy]phenyl}carbamate (760 mg, 85%) as a white solid. LCMS (ESI, m/z): 294.36 [M+H]⁺.

### Step C

A solution of tert-butyl N-{4-[3-(hydroxymethyl)cyclobutoxy]phenyl}carbamate (400 mg, 1.36 mmol, 1.0 equiv) and Dess-Martin reagent (602 mg, 1.42 mmol, 1.04 equiv) in DCM (13 mL) was stirred for 1 hour. Then 5-fluoro-2-[(oxan-4-ylsulfanyl)methyl]-7-(piperidin-4-ylmethoxy)-3H-quinazolin-4-one (562 mg, 1.38 mmol, 1.01 equiv) was added and the solution stirred for 1 hour. Then NaBH₃CN (190 mg, 3.02 mmol, 2.22 equiv) was added at 0° C. and stirred for 1 hour at room temperature. The resulting mixture was concentrated under vacuum and the residue was purified by reversed-phase flash chromatography with the following conditions: (column, C18 silica gel; mobile phase, MeCN in water (10 mmol/L NH₄HCO₃), 0% to 62% gradient in 20 min; detector, UV 254 nm) to afford tert-butyl N-{4-[3-({4-[({5-fluoro-2-[(oxan-4-ylsulfanyl)methyl]-4-oxo-3H-quinazolin-7-yl}oxy)methyl]piperidin-1-yl}methyl)cyclobutoxy]phenyl}carbamate (400 mg, 43%) as a white solid. LCMS (ESI, m/z): 683.85 [M+H]⁺.

### Step D

A solution of tert-butyl N-{4-[3-({4-[({5-fluoro-2-[(oxan-4-ylsulfanyl)methyl]-4-oxo-3H-quinazolin-7-yl}oxy)methyl]piperidin-1-yl}methyl)cyclobutoxy]phenyl}carbamate (400 mg, 0.59 mmol, 1.0 equiv) in DCM (5 mL) and TFA (5 mL) was stirred for 1 hour. After concentration, the residue was purified by reversed-phase flash chromatography with the following conditions: (column, C18 silica gel; mobile phase, MeCN in water (10 mmol/L NH₄HCO₃), 0% to 45% gradient in 15 min; detector, UV 254 nm) to afford 7-[(1-{[3-(4-aminophenoxy)cyclobutyl]methyl}piperidin-4-yl)methoxy]-5-fluoro-2-[(oxan-4-ylsulfanyl)methyl]-3H-quinazolin-4-one (240 mg, 70%) as a white solid. LCMS (ESI, m/z): 583.74 [M+H]⁺.

### Step E

A solution of 7-[(1-{[3-(4-aminophenoxy)cyclobutyl]methyl}piperidin-4-yl)methoxy]-5-fluoro-2-[(oxan-4-ylsulfanyl)methyl]-3H-quinazolin-4-one (240 mg, 0.41 mmol, 1.0 equiv), 3-bromopiperidine-2,6-dione (159 mg, 0.83 mmol, 2.01 equiv) and DIEA (160 mg, 1.24 mmol, 3.01 equiv) in NMP (6 mL) was stirred overnight at 80° C. The mixture was purified by reversed-phase flash chromatography with the following conditions: column, C18 silica gel; mobile phase, MeCN in water (10 mmol/L NH₄HCO₃), 0% to 55% gradient in 20 min; detector, UV 254 nm to afford crude product (210 mg) that was purified by prep-HPLC with the following conditions (Column: Xcelect CSH F-pheny OBD Column, 19*250 mm, 5 μm; Mobile Phase A: Water (10 mmol/L NH₄HCO₃), Mobile Phase B: ACN; Flow rate: 25 mL/min; Gradient: 35% B to 55% B in 10 min, 55% B; Wave Length: 254/220 nm; RT (min): 10.5) to afford crude product. The crude product (70 mg) was further purified by prep-HPLC with the following conditions (Column: XSelect CSH Prep C18 OBD Column, 19*250 mm, 5 μm; Mobile Phase A: Water (10 mmol/L NH₄HCO₃+0.1% NH₃·H₂O), Mobile Phase B: ACN; Flow rate: 25 mL/min; Gradient: 35% B to 55% B in 10 min, 55% B; Wave Length: 254/220 nm; RT (min): 10.5) to afford 3-({4-[3-({4-[({5-fluoro-2-[(oxan-4-ylsulfanyl)methyl]-4-oxo-3H-quinazolin-7-yl}oxy)methyl]piperidin-1-yl}methyl)cyclobutoxy]phenyl}amino)piperidine-2,6-dione (57 mg, 19%) as a white solid. LCMS (ESI, m/z): 694.30 [M+H]⁺. ¹H NMR (300 MHz, DMSO-d₆) δ 12.16 (s, 1H), 10.76 (s, 1H), 6.94-6.83 (m, 2H), 6.61 (d, J=2.9 Hz, 4H), 5.43 (d, J=7.4 Hz, 1H), 4.68-4.34 (m, 1H), 4.21-4.17 (m, 1H), 3.97 (d, J=5.7 Hz, 2H), 3.82-2.76 (m, 2H), 3.62 (s, 2H), 3.37 (d, J=2.3 Hz, 1H), 3.29 (d, J=2.3 Hz, 1H), 3.11-3.01 (m, 1H), 2.90-2.80 (m, 2H), 2.80-2.65 (m, 2H) 2.44-2.34 (m, 2H), 2.15-2.02 (m, 3H), 1.98-1.76 (m, 5H), 1.74-1.58 (m, 5H), 1.54-1.40 (m, 3H), 1.30-1.26 (m, 2H).

### Example 48: Synthesis of 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione

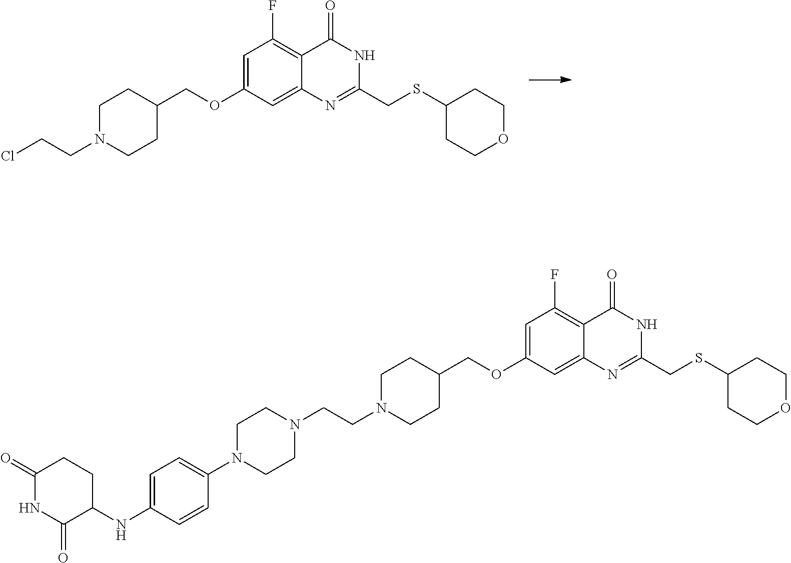 [SMILES: O=c1[nH]c(CSC2CCOCC2)nc2cc(OCC3CCN(CCCl)CC3)cc(F)c12 ; O=C1CCC(Nc2ccc(N3CCN(CCN4CCC(COc5cc(F)c6c(=O)[nH]c(CSC7CCOCC7)nc6c5)CC4)CC3)cc2)C(=O)N1]

A solution of 7-((1-(2-chloroethyl)piperidin-4-yl)methoxy)-5-fluoro-2-(((tetrahydro-2H-pyran-4-yl)thio)methyl)quinazolin-4(3H)-one (124 mg, 0.26 mmol, 1.0 equiv), DIEA (102 mg, 0.79 mmol, 3.0 equiv) and 3-((4-(piperazin-1-yl)phenyl)amino)piperidine-2,6-dione (76.1 mg, 0.26 mmol, 1.0 equiv) in DMSO (3 mL) was stirred for 1 hour at 80° C. The mixture was purified by reverse phase flash chromatography with the following conditions: column, C18 silica gel; mobile phase, MeCN in water (10 mmol/L NH₄HCO₃), 10% to 50% gradient in 10 min; detector, UV 254 nm to afford crude product. The crude product was purified by prep-HPLC with the following conditions (Column: XBridge Prep Phenyl OBD Column, 19*250 mm, 5 μm; Mobile Phase A: Water (10 mmol/L NH₄HCO₃), Mobile Phase B: MeOH; Flow rate: 20 mL/min; Gradient: 60% B to 70% B in 10 min, 70% B; Wave Length: 254 nm; RT (min): 9) to afford 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione (22.4 mg, 10%) as a white solid. LCMS (ESI, m/z): 722.35 [M+H]⁺. ¹H NMR (300 MHz, DMSO-d₆) 6 ¹H NMR (300 MHz, DMSO-d₆) δ 12.13 (s, 1H), 10.73 (s, 1H), 6.94-6.82 (m, 2H), 6.75 (d, J=8.9 Hz, 2H), 6.61 (d, J=8.9 Hz, 2H), 5.35 (d, J=7.3 Hz, 1H), 4.25-4.10 (m, 1H), 3.94-3.75 (m, 2H), 3.62 (s, 2H), 3.40-3.37 (m, 2H), 3.29-3.25 (m, 1H), 3.14-3.00 (m, 1H), 2.98-2.84 (m, 6H), 2.80-2.70 (m, 1H), 2.69-2.63 (m, 1H), 2.65-2.61 (m, 2H), 2.50-2.45 (m, 4H), 2.15-2.05 (m, 1H), 1.98-1.83 (m, 5H), 1.80-1.75 (m, 4H), 1.55-1.20 (m, 5H), 1.11-1.05 (m, 1H).

### Example 48a and 48b: Synthesis of (S)-3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione and (R)-3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione

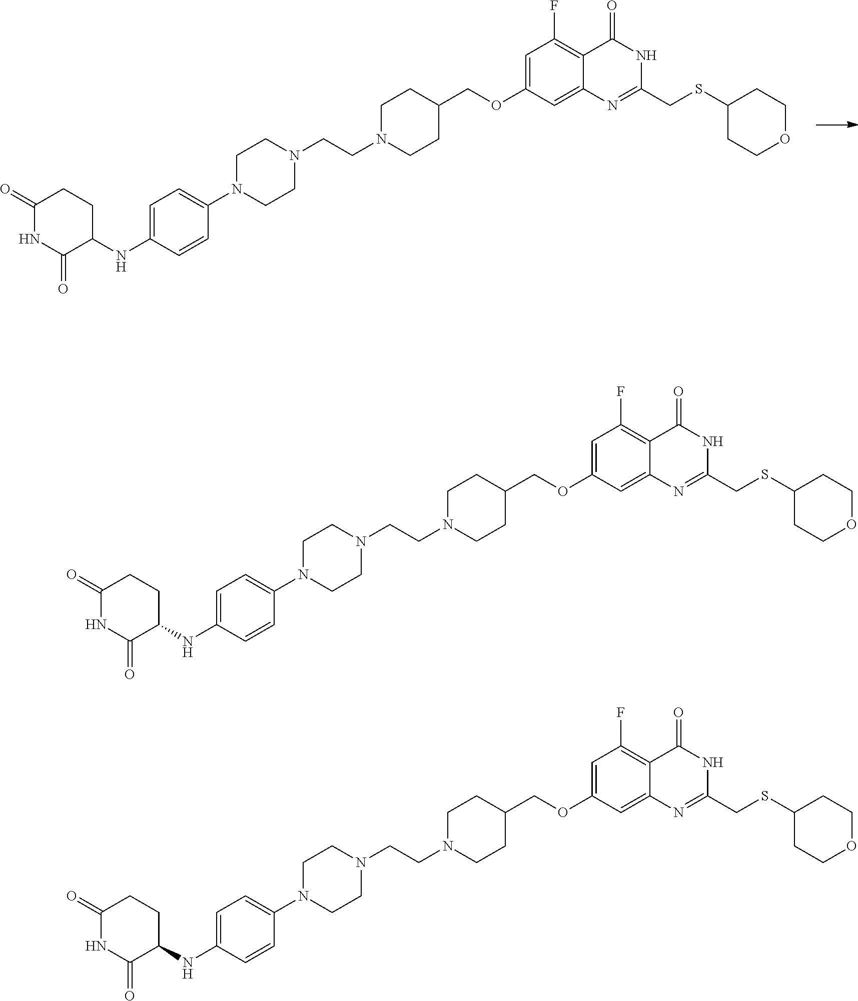 [SMILES: O=C1CCC(Nc2ccc(N3CCN(CCN4CCC(COc5cc(F)c6c(=O)[nH]c(CSC7CCOCC7)nc6c5)CC4)CC3)cc2)C(=O)N1 ; O=C1CC[C@H](Nc2ccc(N3CCN(CCN4CCC(COc5cc(F)c6c(=O)[nH]c(CSC7CCOCC7)nc6c5)CC4)CC3)cc2)C(=O)N1 ; O=C1CC[C@@H](Nc2ccc(N3CCN(CCN4CCC(COc5cc(F)c6c(=O)[nH]c(CSC7CCOCC7)nc6c5)CC4)CC3)cc2)C(=O)N1]

50 mg of the racemic 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione was separated by chiral HPLC with the following conditions: Column: CHIRALPAK IG, 2*25 cm, 5 μm; Mobile Phase A: Hexane (0.5% 2M NH₃-MeOH), Mobile Phase B: EtOH:DCM=1:1; Flow rate: 20 mL/min; Gradient: 9.0% B to 90% B in 24 min; Wave Length: 254/220 nm; RT1: 8.13 min (Example 48a); RT2: 20.70 min (Example 48b); Sample dissolved in MeOH:DCM=1:1; Injection Volume: 3 mL; Number Of Runs: 2. The fractions were directly concentrated and then lyophilized to afford the title compounds. Absolute stereochemistry was not assigned.

Example 48a: First eluting isomer (5.4 mg) isolated using above conditions as a grey solid. LCMS (ESI, m/z): 722.40 [M+H]⁺. 1H NMR (300 MHz, DMSO-d6) δ 12.14 (s, 1H), 10.73 (s, 1H), 6.93-6.83 (m, 2H), 6.75 (d, J=8.8 Hz, 2H), 6.61 (d, J=9.0 Hz 2H), 5.35 (d, J=7.2 Hz, 1H), 4.24-4.13 (m, 1H), 3.98 (d, J=5.8 Hz, 2H), 3.82 (d, J=11.6 Hz, 2H), 3.62 (s, 2H), 3.53-3.41 (m, 3H), 3.11-3.01 (m, 1H), 2.99-2.86 (m, 5H), 2.78-2.58 (m, 3H), 2.57-2.53 (m, 3H), 2.49-2.48 (m, 1H), 2.50-2.45 (m, 3H), 2.18-2.05 (m, 1H), 2.04-1.83 (m, 5H), 1.83-1.65 (m, 3H), 1.55-1.41 (m, 2H), 1.41-1.18 (m, 2H).

Example 48b: Second eluting isomer isolated using above conditions (3.6 mg) as a grey solid. LCMS (ESI, m/z): 722.40 [M+H]⁺. 1H NMR (300 MHz, DMSO-d6) δ 12.14 (s, 1H), 10.73 (s, 1H), 6.93-6.82 (m, 2H), 6.75 (d, J=8.8 Hz, 2H), 6.61 (d, J=9.0 Hz, 2H), 5.35 (d, J=7.2 Hz, 1H), 4.22-4.15 (m, 1H), 3.98 (d, J=5.9 Hz, 2H), 3.88-3.73 (m, 2H), 3.62 (s, 2H), 3.40-3.30 (m, 3H), 3.13-3.01 (m, 1H), 2.95-2.80 (m, 5H), 2.76-2.60 (m, 2H), 2.60-2.35 (m, 9H), 2.15-1.85 (m, 5H), 1.85-1.75 (m, 3H), 1.48-1.20 (m, 4H).

Examples 49 and 50 were synthesized according to the procedure described for the synthesis 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione (Example 48) using appropriate building blocks and modified reaction conditions (such as reagents, reagent ratio, temperature, and reaction time) and purification conditions as needed.

| Example | Structure | Analytical Data |
|---|---|---|
| 49 | 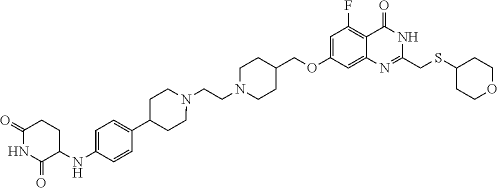 [SMILES: O=C1CCC(Nc2ccc(C3CCN(CCN4CCC(COc5cc(F)c6c(=O)[nH]c(CSC7CCOCC7)nc6c5)CC4)CC3)cc2)C(=O)N1] 3-((4-(1-(2-(4-(((5-fluoro-4-oxo-2-(((tetrahydro- 2H-pyran-4-yl)thio)methyl)-3,4- dihydroquinazolin-7-yl)oxy)methyl)piperidin-1-yl)ethyl) piperidin-4-yl)phenyl)amino)piperidine-2,6-dione | LCMS (ESI, m/z): 721.10 [M + H]⁺. ¹HNMR (300 MHz, DMSO-d₆) δ 12.13 (s, 1H), 10.75 (s, 1H), 6.95 (d, J = 8.4 Hz, 2H), 6.87 (d, J = 8.1 Hz, 2H), 6.61 (d, J = 8.4 Hz, 2H), 5.62 (d, J = 7.4 Hz, 1H), 4.41 − 4.29 (m, 1H), 3.98 (d, J = 5.9 Hz, 2H), 3.87 − 3.77 (m, 2H), 3.62 (s, 2H), 3.41 − 3.29 (m, 1H), 3.12 − 3.01(m, 1H), 2.93 (t, J = 11.9 Hz, 4H), 2.71 − 2.61 (m, 1H), 2.42 (s, 4H), 1.95 − 1.80 (m, 7H), 1.73 − 1.37 (m, 1H), 1.28 − 1.19(m, 4H). |
| 50 | 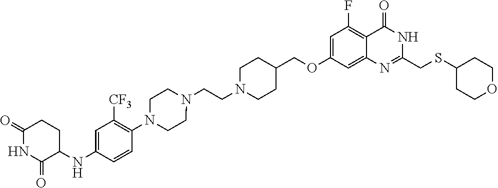 [SMILES: O=C1CCC(Nc2ccc(N3CCN(CCN4CCC(COc5cc(F)c6c(=O)[nH]c(CSC7CCOCC7)nc6c5)CC4)CC3)c(C(F)(F)F)c2)C(=O)N1] 3-((4-(4-(2-(4-(((5-fluoro-4-oxo-2-(((tetrahydro- 2H-pyran-4-yl)thio)methyl)-3,4- dihydroquinazolin-7-yl)oxy)methyl)piperidin-1- yl)ethyl)piperazin-1-yl)-3- (trifluoromethyl)phenyl)amino)piperidine-2,6-dione | LCMS (ESI, m/z): 790.40 [M + H]+; H NMR (300 MHz, DMSO-d₆) δ 12.13 (s, 1H), 10.77 (s, 1H), 7.32 (d, J = 8.6 Hz, 1H), 6.95 − 6.84 (m, 4H), 6.17 (d, J = 7.8 Hz, 1H), 4.45 − 4.31 (m, 1H), 3.98 (d, J = 5.8 Hz, 2H), 3.83 (dd, J = 11.5, 3.8 Hz, 2H), 3.62 (s, 2H), 3.40 − 3.35 (m, 3H), 3.12 − 3.00 (m, 1H), 2.92 (d, J = 10.7 Hz, 2H), 2.89 − 2.71 (m, 5H), 2.75 − 2.68 (m, 1H), 2.54 − 2.44 (m, 4H), 2.12 − 2.05 (m, 1H), 2.00 − 1.67 (m, 9H), 1.53 − 1.24 (m, 6H). |

### Example 51: Synthesis of 3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)phenyl)amino)piperidine-2,6-dione

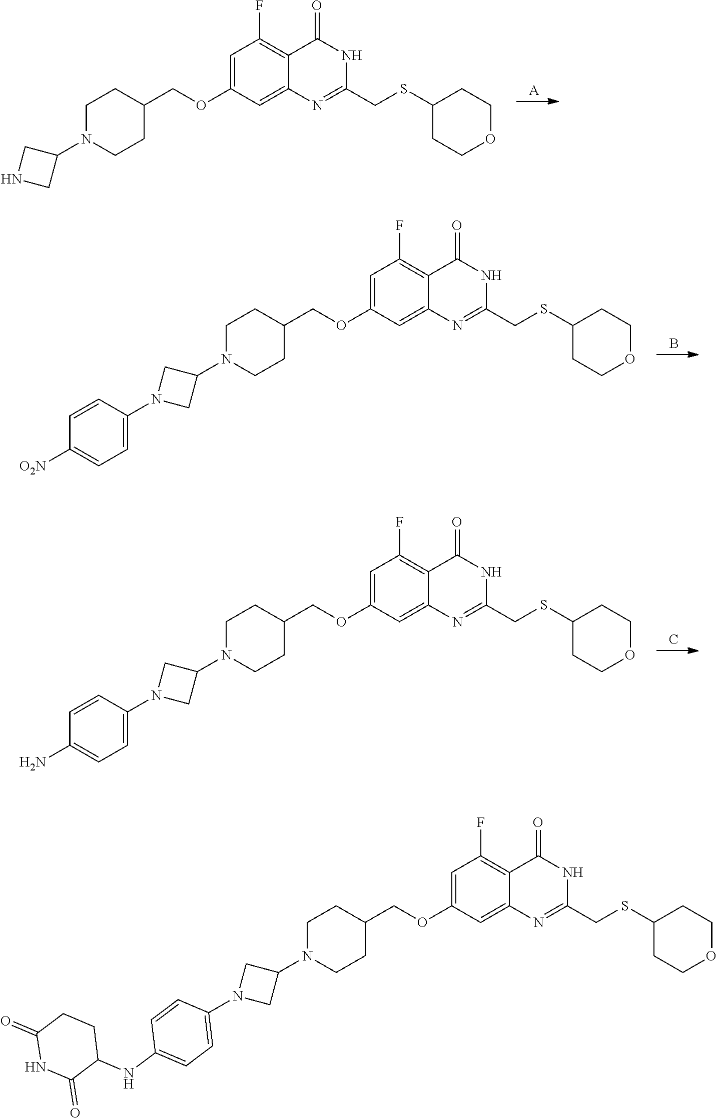

### Step A

A solution of 7-((1-(azetidin-3-yl)piperidin-4-yl)methoxy)-5-fluoro-2-(((tetrahydro-2H-pyran-4-yl)thio)methyl)quinazolin-4(3H)-one (300 mg, 0.65 mmol, 1.0 equiv), 1-fluoro-4-nitrobenzene (91.5 mg, 0.65 mmol, 1.0 equiv), DIEA (251 mg, 1.95 mmol, 3.0 equiv) and NMP (2 mL) was stirred for 2 hours at 80° C. The mixture was purified by reversed-phase flash chromatography with the following conditions: column, C18 silica gel; mobile phase, MeCN in water (10 mmol/L NH₄HCO₃), 10% to 70% gradient in 15 min; detector, UV 254 nm to afford 5-fluoro-7-((1-(1-(4-nitrophenyl)azetidin-3-yl)piperidin-4-yl)methoxy)-2-(((tetrahydro-2H-pyran-4-yl)thio)methyl)quinazolin-4(3H)-one (224 mg, 59%) as a yellow solid. LCMS (ESI, m/z): 584.23 [M+H]⁺.

### Step B

A solution of Fe (107 mg, 1.92 mmol, 5.0 equiv), NH₄Cl (41.1 mg, 0.77 mmol, 2.0 equiv) EtOH (3 mL, 51.6 mmol, 135 equiv) in water (1 mL) was stirred for 10 minutes at 80° C., then 5-fluoro-7-((1-(1-(4-nitrophenyl)azetidin-3-yl)piperidin-4-yl)methoxy)-2-(((tetrahydro-2H-pyran-4-yl)thio)methyl)quinazolin-4(3H)-one (224 mg, 0.38 mmol, 1 equiv) is added portion-wise at 80° C. The mixture was heated in the microwave for 30 mins at 80° C. The resulting solution was filtered at 80° C., the filter cake was washed with EtOH (30 mL). The filtrate was concentrated and purified by reversed-phase flash chromatography with the following conditions: column, C18 silica gel; mobile phase, MeCN in water (10 mmol/L NH₄HCO₃), 10% to 60% gradient in 10 min; detector, UV 254 nm to afford 7-((1-(1-(4-aminophenyl)azetidin-3-yl)piperidin-4-yl)methoxy)-5-fluoro-2-(((tetrahydro-2H-pyran-4-yl)thio)methyl)quinazolin-4(3H)-one (177 mg, 83%) as a yellow solid. LCMS (ESI, m/z): 554.25 [M+H]⁺.

### Step C

A solution of 7-((1-(1-(4-aminophenyl)azetidin-3-yl)piperidin-4-yl)methoxy)-5-fluoro-2-(((tetrahydro-2H-pyran-4-yl)thio)methyl)quinazolin-4(3H)-one (177 mg, 0.32 mmol, 1.0 equiv), 3-bromopiperidine-2,6-dione (73.7 mg, 0.38 mmol, 1.2 equiv), DIEA (82.6 mg, 0.64 mmol, 2.0 equiv) and NMP (2 mL) was stirred for 3 hours at 80° C. The mixture was purified by reversed-phase flash chromatography with the following conditions: (column, C18 silica gel; mobile phase, MeCN in water (10 mmol/L NH₄HCO₃), 10% to 50% gradient in 10 min; detector, UV 254 nm) and further purified by prep-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 32% B to 40% B in 8 min, 40% B; Wave Length: 220/254 nm; RT (min): 5.84) to afford 3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)phenyl)amino)piperidine-2,6-dione (31.5 mg, 15%) as a purple solid. LCMS (ESI, m/z): 665.35 [M+H]⁺; ¹HNMR (300 MHz, DMSO-d₆) δ 12.12 (s, 1H), 10.72 (s, 1H), 6.93-6.82 (m, 2H), 6.61 (d, J=8.7 Hz, 2H), 6.30 (d, J=8.6 Hz, 2H), 5.17 (d, J=7.0 Hz, 1H), 4.18-4.06 (m, 1H), 3.98 (d, J=5.8 Hz, 2H), 3.88-3.75 (m, 4H), 3.66-3.56 (m, 3H), 3.20-3.12 (m, 1H), 3.11-2.99 (m, 1H), 2.87-2.59 (m, 4H), 2.20-2.00 (m, 1H), 1.94-1.70 (m, 8H), 1.56-1.20 (m, 5H).

Examples 52 and 53 were synthesized according to the procedure described for the synthesis 3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)phenyl)amino)piperidine-2,6-dione (Example 51) using appropriate building blocks and modified reaction conditions (such as reagents, reagent ratio, temperature, and reaction time) and purification conditions as needed.

| Example | Structure | Analytical Data |
|---|---|---|
| 52 | 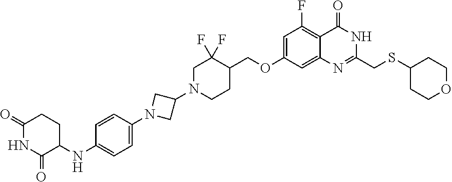 [SMILES: O=C1CCC(Nc2ccc(N3CC(N4CCC(COc5cc(F)c6c(=O)[nH]c(CSC7CCOCC7)nc6c5)C(F)(F)C4)C3)cc2)C(=O)N1] 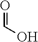 | LCMS (ESI, m/z): 701.25 [M + H]⁺. ¹H NMR (300 MHz, DMSO-d₆) δ 12.17 (s, 1H), 10.80 (br, 1H), 8.18 (s, ¹H, HCOOH), 6.91 (s, 2H), 6.85(s, 1H), 6.84-6.23 (m, 3H), 4.39 (dd, J = 10.3, 5.3 Hz, 2H), 4.13 (dd, J = 10.3, 6.8 Hz, 2H), 3.89-3.79 (m, 4H), 3.63 (s, 3H), 3.39-3.30 (m, 1H), 3.15-2.95 (m, 3H), 2.90-2.65 (m, 3H), 2.48- 2.05 (m, 3H), 2.05-1.80 (m, 5H), 1.69-1.39 (m, 4H). |
| | 3-((4-(3-(3,3-difluoro-4-(((5-fluoro-4-oxo-2- | |
| | (((tetrahydro-2H-pyran-4-yl)thio)methyl)-3,4- | |
| | dihydroquinazolin-7-yl)oxy)methyl)piperidin-1- | |
| | yl)azetidin-1-yl)phenyl)amino)piperidine-2,6- | |
| | dione formate salt | |
| 53 | 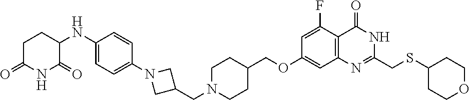 [SMILES: O=C1CCC(Nc2ccc(N3CC(CN4CCC(COc5cc(F)c6c(=O)[nH]c(CSC7CCOCC7)nc6c5)CC4)C3)cc2)C(=O)N1] 3-((4-(3-((4-(((5-fluoro-4-oxo-2-(((tetrahydro-2H- pyran-4-yl)thio)methyl)-3,4-dihydroquinazolin-7- yl)oxy)methyl)piperidin-1-yl)methyl)azetidin-1- yl)phenyl)amino)piperidine-2,6-dione | LCMS (ESI, m/z): 679.30 [M + H]⁺. ¹H NMR (300 MHz, DMSO-d₆) δ 12.19 (s, 1H), 10.76 (s, 1H), 8.26 (s, 1H), 6.95-6.84 (m, 2H), 6.60 (d, J = 8.5 Hz, 2H), 6.32-6.23 (d, J = 8.5 Hz, 2H), 5.19 (s, 1H), 4.19- 4.09 (m, 1H), 3.98 (d, J = 5.8 Hz, 2H), 3.87-3.74 (m, 5H), 3.62 (s, 2H), 3.11- 2.99 (m, 2H), 2.88-2.80 |
| | | (m, 4H), 2.80-2.65 (m, |
| | | 1H), 2.64-2.55 (m, 9H), |
| | | 2.11-2.01(m, 1H), 1.96- |
| | | 1.83 (m, 6H), 1.82-1.68 |
| | | (m, 4H), 1.50-1.18 (m, 8H). |

### Example 54: Synthesis of 1-(3-(3-((4-(((5-fluoro-4-oxo-2-(((tetrahydro-2H-pyran-4-yl)thio)methyl)-3,4-dihydroquinazolin-7-yl)oxy)methyl)piperidin-1l-yl)methyl)azetidin-1l-yl)phenyl)dihydropyrimnidine-2,4(1H,3H)-dione formate salt

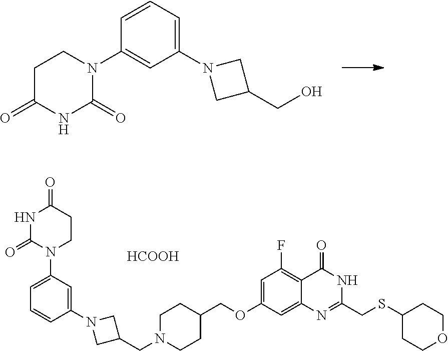 [SMILES: O=C1CCN(c2cccc(N3CC(CO)C3)c2)C(=O)N1 ; O=C1CCN(c2cccc(N3CC(CN4CCC(COc5cc(F)c6c(=O)[nH]c(CSC7CCOCC7)nc6c5)CC4)C3)c2)C(=O)N1 ; "HCOOH"]

A solution of 1-(3-(3-(hydroxymethyl)azetidin-1-yl)phenyl)dihydropyrimidine-2,4(1H,3H)-dione (100 mg, 0.363 mmol, 1.0 equiv), Dess-Martin periodane (169 mg, 0.399 mmol, 1.1 equiv) and DCM (3 mL) was stirred for 2 hours. Then 5-fluoro-7-(piperidin-4-ylmethoxy)-2-(((tetrahydro-2H-pyran-4-yl)thio)methyl)quinazolin-4(3H)-one (148.02 mg, 0.363 mmol, 1 equiv) was added dropwise over 1 minute. The resulting mixture was stirred for 1 hour and then STAB (385 mg, 1.82 mmol, 5.0 equiv) was added over 1 min. The resulting mixture was stirred for 2 hours and then was concentrated under reduced pressure. The residue was purified by silica gel column chromatography, eluting with PE/EA (60%: 40%) to afford the crude product that was further purified by prep-HPLC with the following conditions (Column: Xselect CSH C18 OBD Column 30*150 mm 5 μm, n; Mobile Phase A: water (0.1% FA), Mobile Phase B: ACN; Flow rate: 60 mL/min; Gradient: 10% B to 20% B in 10 min, 20% B; Wave Length: 254/220 nm; RT (min): 8.68) to afford 1-(3-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)phenyl)dihydropyrimidine-2,4(1H,3H)-dione formate salt (20 mg, 8%) as an off-white solid. LCMS(ES, m/z): 665.40 [M+H]⁺. ¹H NMR (400 MHz, DMSO-d₆) δ 12.17 (s, 1H), 10.29 (s, 1H), 8.21 (d, J=2.0 Hz, 1H), 7.15 (t, J=8.0 Hz, 1H), 6.92-6.84 (m, 2H), 6.60 (dd, J=7.8, 1.9 Hz, 1H), 6.44-6.13 (m, 2H), 4.01-3.96 (m, 2H), 3.94-3.88 (m, 2H), 3.85-3.79 (m, 2H), 3.76-3.70 (m, 2H), 3.63-3.60 (m, 2H), 3.36-3.29 (m, 4H), 3.11-3.01 (m, 1H), 2.99-2.81 (m, 3H), 2.71-2.65 (m, 2H), 2.63-2.55 (m, 2H), 2.04-1.93 (m, 2H), 1.92-1.85 (m, 2H), 1.82-1.68 (m, 3H), 1.51-1.39 (m, 2H), 1.38-1.24 (m, 2H).

### Example 55: Synthesis of 3-((4-(4-(2-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione

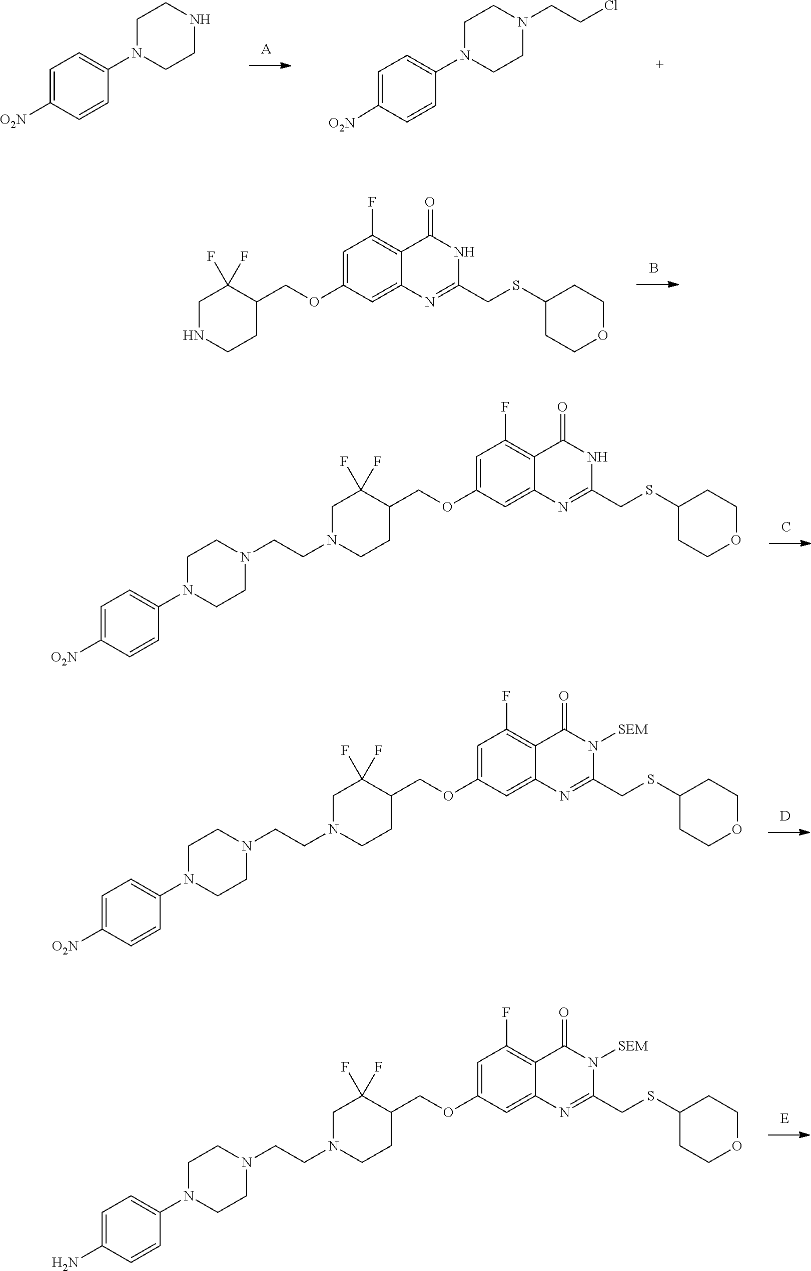

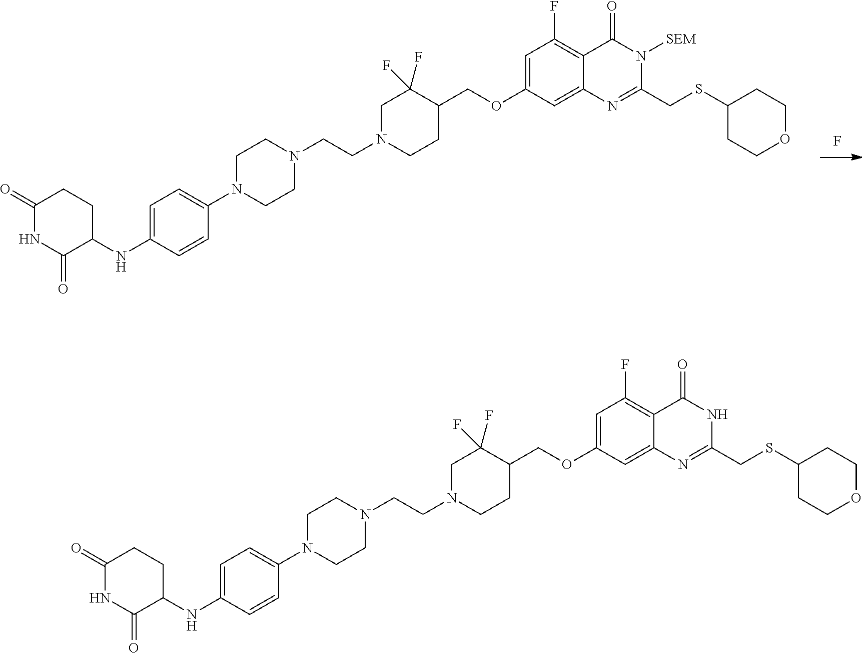

### Step A

A solution of 1-(4-nitrophenyl)piperazine (2.0 g, 9.65 mmol, 1.0 equiv), 2-chloroacetaldehyde (3.79 g, 48.3 mmol, 5.0 equiv), STAB (4.09 g, 19.3 mmol, 2.0 equiv) in DCM (10 mL) was stirred for 2 hours. The resulting mixture was concentrated under reduced pressure.

The residue was purified by silica gel column chromatography, eluting with PE/EtOAc (52:48) to afford 1-(2-chloroethyl)-4-(4-nitrophenyl)piperazine (2.5 g, 96%) as a yellow solid. LCMS (ESI, m/z): 270.09 [M+H]⁺.

### Step B

A solution of 7-((3,3-difluoropiperidin-4-yl)methoxy)-5-fluoro-2-(((tetrahydro-2H-pyran-4-yl)thio)methyl)quinazolin-4(3H)-one (500 mg, 0.34 mmol, 1.0 equiv), 1-(2-chloroethyl)-4-(4-nitrophenyl)piperazine (395 mg, 1.47 mmol, 1.3 equiv), and K₂CO₃ (312 mg, 2.25 mmol, 2.0 equiv) in ACN (2 mL) was stirred overnight at 80° C. The resulting mixture was concentrated under reduced pressure. The residue was purified by silica gel column chromatography, eluting with CH₂Cl₂/MeOH (97:3) to afford 7-((3,3-difluoro-1-(2-(4-(4-nitrophenyl)piperazin-1-yl)ethyl)piperidin-4-yl)methoxy)-5-fluoro-2-(((tetrahydro-2H-pyran-4-yl)thio)methyl)quinazolin-4(3H)-one (216 mg, 28%) as a yellow solid. LCMS (ESI, m/z): 677.25 [M+H]⁺.

### Step C

A solution of 7-((3,3-difluoro-1-(2-(4-(4-nitrophenyl)piperazin-1-yl)ethyl)piperidin-4-yl)methoxy)-5-fluoro-2-(((tetrahydro-2H-pyran-4-yl)thio)methyl)quinazolin-4(3H)-one (210 mg, 0.31 mmol, 1.0 equiv), SEM-Cl (155 mg, 0.93 mmol, 3.0 equiv), and Cs₂CO₃ (303 mg, 0.93 mmol, 3.0 equiv) in THF (4 ml) was stirred for 1 hour. The resulting mixture was concentrated under reduced pressure. The residue was purified by silica gel column chromatography, eluting with CH₂Cl₂/MeOH (98:2) to afford 7-((3,3-difluoro-1-(2-(4-(4-nitrophenyl)piperazin-1-yl)ethyl)piperidin-4-yl)methoxy)-5-fluoro-2-(((tetrahydro-2H-pyran-4-yl)thio)methyl)-3-((2-(trimethylsilyl)ethoxy)methyl)quinazolin-4(3H)-one (102 mg, 41%) as a yellow solid. LCMS (ESI, m/z): 807.35 [M+H]⁺.

### Step D

A solution of Fe (101 mg, 1.81 mmol, 10 equiv), NH₄Cl (12.9 mg, 0.24 mmol, 2.0 equiv), EtOH (3 mL), and water (1 mL) was stirred for 10 min at 80° C. and then 7-((3,3-difluoro-1-(2-(4-(4-nitrophenyl)piperazin-1-yl)ethyl)piperidin-4-yl)methoxy)-5-fluoro-2-(((tetrahydro-2H-pyran-4-yl)thio)methyl)-3-((2-(trimethylsilyl)ethoxy)methyl)quinazolin-4(3H)-one (97 mg, 0.18 mmol, 1.0 equiv) was added in portions at 80° C. The insoluble solids were filtered out at 80° C. and the filter cake was washed with EtOH (30 mL). The filtrate was concentrated and the residue was purified by silica gel column chromatography, eluting with CH₂Cl₂/MeOH (95:5) to afford 7-((1-(2-(4-(4-aminophenyl)piperazin-1-yl)ethyl)-3,3-difluoropiperidin-4-yl)methoxy)-5-fluoro-2-(((tetrahydro-2H-pyran-4-yl)thio)methyl)-3-((2-(trimethylsilyl)ethoxy)methyl)quinazolin-4(3H)-one (70 mg, 75%) as a yellow solid. LCMS (ESI, m/z): 777.40 [M+H]⁺.

### Step E

A solution of 7-((1-(2-(4-(4-aminophenyl)piperazin-1-yl)ethyl)-3,3-difluoropiperidin-4-yl)methoxy)-5-fluoro-2-(((tetrahydro-2H-pyran-4-yl)thio)methyl)-3-((2-(trimethylsilyl)ethoxy)methyl)quinazolin-4(3H)-one (65 mg, 0.084 mmol, 1.0 equiv), 3-bromopiperidine-2,6-dione (32 mg, 0.17 mmol, 2.0 equiv), NaHCO₃ (14 mg, 0.17 mmol, 2.0 equiv) in ACN (1.5 mL) was stirred for 3 days at 90° C. The resulting mixture was concentrated under reduced pressure. The residue was purified by silica gel column chromatography, eluting with CH₂Cl₂/MeOH (96:4) to afford 3-((4-(4-(2-(3,3-difluoro-4-(((5-fluoro-4-oxo-2-(((tetrahydro-2H-pyran-4-yl)thio)methyl)-3-((2-(trimethylsilyl)ethoxy)methyl)-3,4-dihydroquinazolin-7-yl)oxy)methyl)piperidin-1-yl)ethyl)piperazin-1-yl)phenyl)amino)piperidine-2,6-dione (36 mg, 48%) as a blue solid. LCMS (ESI, m/z): 888.40 [M+H]⁺.

### Step F

A solution of 3-((4-(4-(2-(3,3-difluoro-4-(((5-fluoro-4-oxo-2-(((tetrahydro-2H-pyran-4-yl)thio)methyl)-3-((2-(trimethylsilyl)ethoxy)methyl)-3,4-dihydroquinazolin-7-yl)oxy)methyl)piperidin-1-yl)ethyl)piperazin-1-yl)phenyl)amino)piperidine-2,6-dione (35 mg, 0.039 mmol, 1.0 equiv) in TFA (0.5 mL) and DCM (2.5 mL) was stirred for 2 hours. The resulting mixture was concentrated under reduced pressure. The crude product (45 mg) was purified by prep-HPLC with the following conditions (Column: Xselect CSH C18 OBD Column 30*150 mm, 5 μm; Mobile Phase A: water (0.1% FA), Mobile Phase B: ACN; Flow rate: 60 mL/min; Gradient: 14% B to 24% B in 10 min, 24% B; Wave Length: 254/220 nm; RT1 (min): 7.57) to afford 3-((4-(4-(2-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione (7.2 mg, 24%) as an off-white solid. LCMS (ESI, m/z): 759.25 [M+H]⁺. ¹H NMR (300 MHz, DMSO-d₆) δ 12.17 (s, 1H), 10.73 (s, 1H), 6.92 (s, 1H), 6.90 (s, 1H), 6.75 (d, J=8.9 Hz, 2H), 6.66-6.57 (m, 2H), 5.35 (d, J=7.1 Hz, 1H), 4.38 (dd, J=10.4, 5.3 Hz, 1H), 4.29-4.06 (m, 3H), 3.76-3.89 (m, 3H), 3.62 (s, 2H), 3.53-3.50 (m, 1H), 3.22-3.29 (m, 3H), 3.19-3.01 (m, 2H), 2.95-2.89 (m, 4H), 2.80-2.66 (m, 1H), 2.62-2.54 (m, 6H), 2.49-2.26 (m, 2H), 2.27-2.06 (m, 1H), 2.01-1.80 (m, 3H), 1.62-1.32 (m, 3H), 1.28-1.20 (m, 2H).

### Example 56: Synthesis of 3-((4-(4-(3-(((5-fluoro-4-oxo-2-(((1-(2,2,2-trifluoroethyl)piperidin-4-yl)thio)methyl)-3,4-dihydroquinazolin-7-yl)oxy)methyl)cyclobutyl)piperazin-1-yl)phenyl)amino)piperidine-2,6-dione

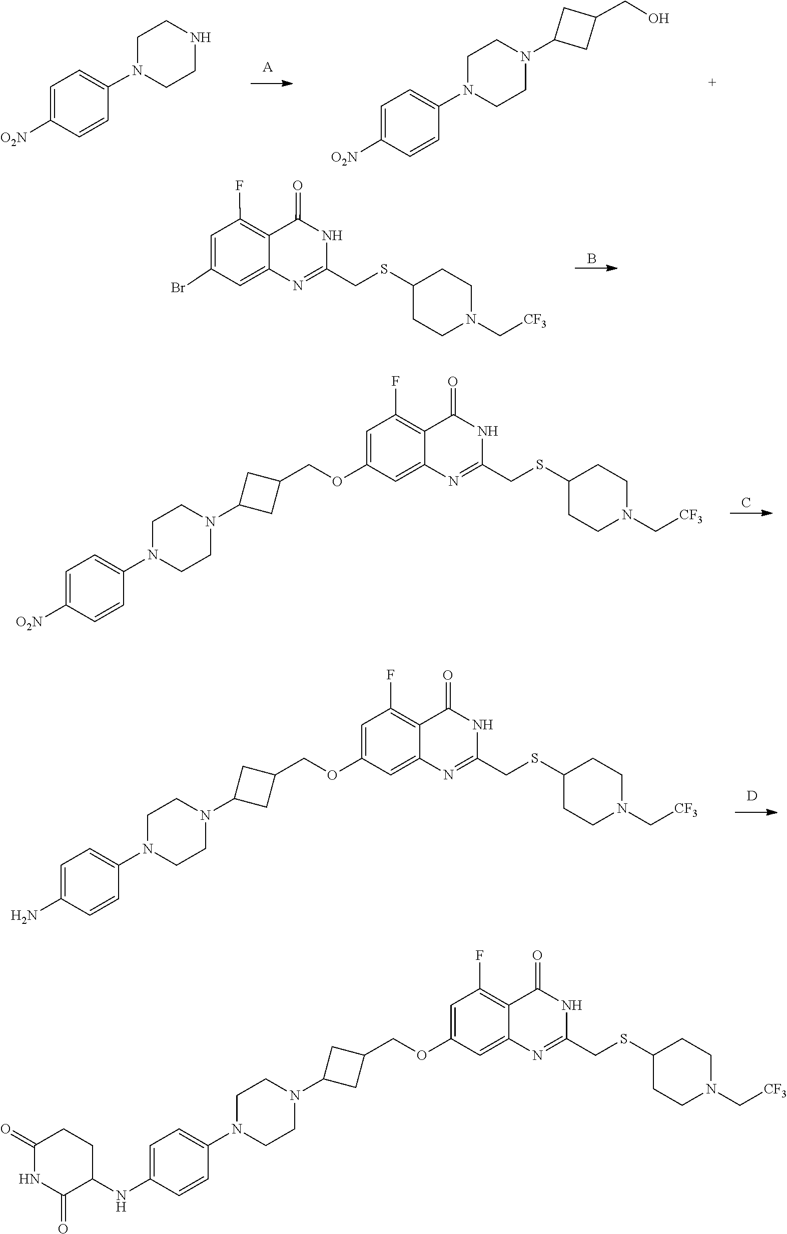

### Step A

A solution of 1-(4-nitrophenyl)piperazine (620 mg, 2.99 mmol, 1.0 equiv) and 3-(hydroxymethyl)cyclobutan-1-one (449 mg, 4.98 mmol, 1.5 equiv) and STAB (1270 mg, 5.98 mmol, 2.0 equiv) in DCE (15 mL) was stirred overnight under nitrogen atmosphere. The resulting mixture was concentrated under reduced pressure. The residue was purified by silica gel column chromatography, eluting with DCM to afford (3-(4-(4-nitrophenyl)piperazin-1-yl)cyclobutyl)methanol (940 mg) as a yellow solid which was used in the next step without purification. LCMS (ESI, m/z): 292.16 [M+H]⁺.

### Step B

A solution of 7-bromo-5-fluoro-2-(((1-(2,2,2-trifluoroethyl)piperidin-4-yl)thio)methyl)quinazolin-4(3H)-one (260 mg, 0.57 mmol, 1.0 equiv), (3-(4-(4-nitrophenyl)piperazin-1-yl)cyclobutyl)methanol (183 mg, 0.63 mmol, 1.1 equiv), Cs₂CO₃ (373 mg, 1.14 mmol, 2.0 equiv), RockPhos (26.8 mg, 0.057 mmol, 0.1 equiv) and Pd₂(allyl)₂Cl₂ (2.09 mg, 0.006 mmol, 0.01 equiv) in toluene (5 mL) was stirred for 2 days at 100° C. under nitrogen atmosphere. The resulting mixture was concentrated under reduced pressure. The residue was purified by reverse phase flash chromatography with the following conditions: column, C18 silica gel; mobile phase, MeCN in water (10 mmol/L NH₄HCO₃), 10% to 65% gradient in 20 mins; detector, UV 254 nm. This afforded 5-fluoro-7-((3-(4-(4-nitrophenyl)piperazin-1-yl)cyclobutyl)methoxy)-2-(((1-(2,2,2-trifluoroethyl)piperidin-4-yl)thio)methyl)quinazolin-4(3H)-one (75 mg, 19%) as a brown solid. LCMS (ESI, m/z): 665.25 [M+H]⁺.

### Step C

A solution of 5-fluoro-7-((3-(4-(4-nitrophenyl)piperazin-1-yl)cyclobutyl)methoxy)-2-(((1-(2,2,2-trifluoroethyl)piperidin-4-yl)thio)methyl)quinazolin-4(3H)-one (100 mg, 0.150 mmol, 1.0 equiv), Fe (42.0 mg, 0.75 mmol, 5.0 equiv) and NH₄Cl (16.1 mg, 0.300 mmol, 2.0 equiv) in EtOH (3 mL) and water (0.6 mL) was stirred overnight at 80° C. The resulting mixture was filtered at 80° C. and the filter cake was washed with ethanol (3×5 mL). The filtrate was concentrated under reduced pressure. The residue was purified by reverse phase flash chromatography with the following conditions: column, C18 silica gel; mobile phase, MeCN in water (10 mmol/L NH₄HCO₃), 10% to 51% gradient in 30 min; detector, UV 254 nm. This afforded 7-((3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one (60 mg, 63%) as a black solid. LCMS (ESI, m/z): 635.27 [M+H]⁺.

### Step D

A solution of 7-((3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one (50 mg, 0.079 mmol, 1.0 equiv), 3-bromopiperidine-2,6-dione (23 mg, 0.12 mmol, 1.5 equiv) and NaHCO₃ (20 mg, 0.24 mmol, 3.0 equiv) in ACN (5 mL) was stirred for 48 hours at 90° C. The resulting mixture was concentrated under reduced pressure. The residue was purified by reverse phase flash chromatography with the following conditions: column, C18 silica gel; mobile phase, MeCN in water (10 mmol/L NH₄HCO₃), 10% to 39% gradient in 30 min; detector, UV 254 nm. This afforded 3-((4-(4-(3-(((5-fluoro-4-oxo-2-(((1-(2,2,2-trifluoroethyl)piperidin-4-yl)thio)methyl)-3,4-dihydroquinazolin-7-yl)oxy)methyl)cyclobutyl)piperazin-1-yl)phenyl)amino)piperidine-2,6-dione (16 mg, 28%) as a grey solid. LCMS (ESI, m/z): 373.80 [M12+H]⁺. ¹H NMR (400 MHz, DMSO-d₆) δ 12.09 (s, 1H), 10.75 (s, 1H), 6.91-6.84 (m, 2H), 6.75 (d, J=8.0 Hz, 2H), 6.61 (d, J=8.0 Hz, 2H), 5.37 (d, J=8.0 Hz, 1H), 4.22-4.16 (m, 2H), 4.09 (d, J=8.0 Hz, 1H), 3.60 (s, 2H), 3.30-3.28 (m, 1H), 3.18-3.09 (m, 2H), 3.00-2.90 (m, 4H), 2.80-2.78 (m, 3H), 2.77-2.66 (m, 1H), 2.62-2.56 (m, 1H), 2.44-2.31 (m, 7H), 2.25-2.16 (m, 1H), 2.14-2.02 (m, 2H), 2.00-1.78 (m, 4H), 1.72-1.63 (m, 1H), 1.52-1.42 (m, 2H).

### Example 57a and 57b: Synthesis of 7-(((1s,3s)-3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one and 7-(((1r,3r)-3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one

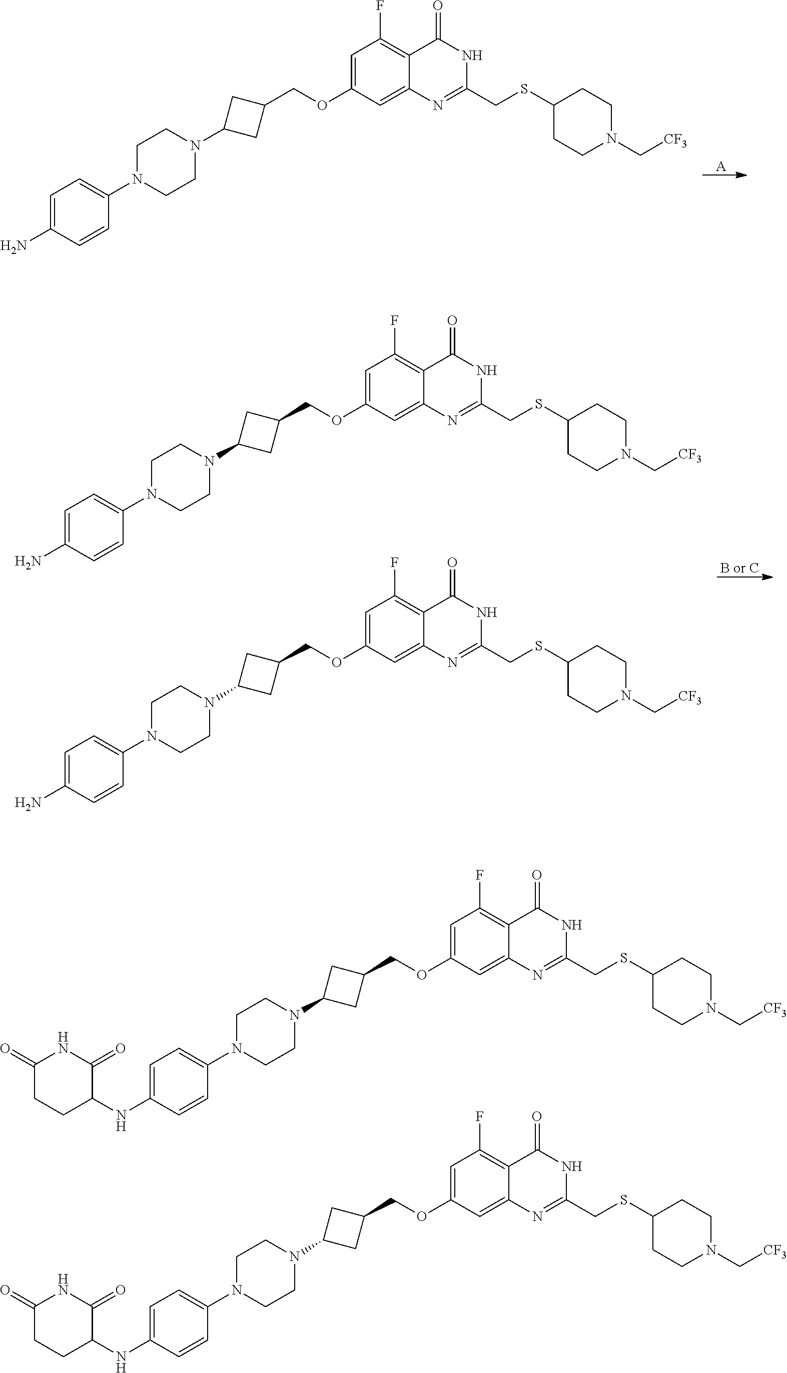

### Step A

374 mg of trans- and cis-mixture of 7-((3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one was separated by prep-HPLC with the following conditions: Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 28% B to 38% B in 10 min; Wave Length: 220/254 nm; RT: 12.47 min (57-A-a) and 14.10 min (57-A-b). This afforded product 57-A-a (52 mg; the first eluting isomer) as a grey solid and product 57-A-b (79 mg; the second eluting isomer) as a grey solid. The stereochemistry of the isomers was not assigned. LCMS (ESI, m/z): 635.20 [M+H]⁺.

### Step B

A solution of product 57-A-a from Step A (52 mg, 0.07 mmol, 1.0 equiv), 3-bromopiperidine-2,6-dione (20 mg, 0.105 mmol, 1.5 equiv), and NaHCO₃ (18 mg, 0.21 mmol, 3.0 equiv) in ACN (6 mL) was stirred for 48 hours at 90° C. The residue was purified by reverse phase flash chromatography with the following conditions: column, C18 silica gel; mobile phase, MeCN in water (10 mmol/L NH₄HCO₃), 10% to 50% gradient in 10 min; detector, UV 254 nm to afford Example 57a (8.7 mg, 15%) as a grey solid. LCMS (ESI, m/z): 746.20[M+H]⁺. ¹H NMR (400 MHz, DMSO-d₆) δ 10.75 (s, 1H), 6.88 (d, J=14.1 Hz, 2H), 6.76 (d, J=8.8 Hz, 2H), 6.62 (d, J=8.9 Hz, 2H), 5.37 (d, J=7.3 Hz, 1H), 4.18 (d, J=7.3 Hz, 3H), 3.60 (s, 2H), 3.13 (t, J=10.3 Hz, 3H), 2.94 (s, 4H), 2.86 (d, J=10-Hz, 2H), 2.40-2.30 (m, 7H), 2.16-2.03 (m, 3H), 1.91-1.71 (m, 4H), 1.53-1.44 (m, 2H).

### Step C

A solution of product 57-A-b from Step A (74 mg, 0.099 mmol, 1.0 equiv), 3-bromopiperidine-2,6-dione (29 mg, 0.15 mmol, 1.5 equiv), and NaHCO₃ (25 mg, 0.30 mmol, 3.0 equiv) in ACN (7 mL) was stirred for 48 hours at 90° C. The residue was purified by reverse phase flash chromatography with the following conditions: column, C18 silica gel; mobile phase, MeCN in water (10 mmol/L NH₄HCO₃), 10% to 50% gradient in 10 min; detector, UV 254 nm. to afford Example 57b (18 mg, 20%) as a grey solid. LCMS (ESI, m/z): 746.20 [M+H]^{+ 1}H NMR (400 MHz, DMSO-d₆) 612.10 (s, 1H), 10.75 (s, 1H), 6.82 (d, J=13.5 Hz, 4H), 6.75 (d, J=9.0 Hz, 2H), 5.37 (d, J=7.2 Hz, 1H), 4.24-4.14 (m, 1H), 4.08 (d, J=6.3 Hz, 2H), 3.58 (s, 2H), 3.14 (q, J=10.3 Hz, 2H), 2.93-2.85 (m, 6H), 2.79-2.68 (m, 1H), 2.63-2.50 (m, 1H), 2.55-2.36 (m, 8H), 2.25-2.05 (m, 3H), 1.92-1.85 (m, 3H), 1.70-1.60 (m, 2H), 1.50-1.35 (m, 2H).

### Example 58: Synthesis of 5-fluoro-3-(4-methoxybenzyl)-7-((1-(2-(4-nitrophenoxy)ethyl)piperidin-4-yl)methoxy)-2-(((tetrahydro-2H-pyran-4-yl)thio)methyl)quinazolin-4(3H)-one

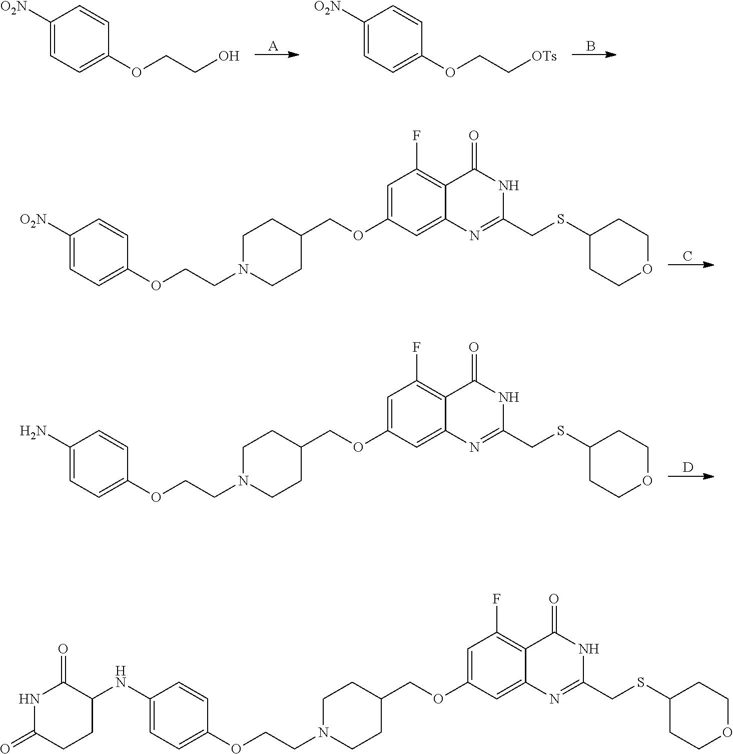

### Step A

To a mixture of 2-(4-nitrophenoxy)ethanol (2.0 g, 10.9 mmol, 1.0 equiv) and p-toluenesulfonyl chloride (4.16 g, 21.8 mmol, 2.0 equiv) in DCM (10 mL) was added TEA (3.3 g, 32.8 mmol, 3.0 equiv) and DMAP (133 mg, 1.09 mmol, 0.1 equiv) at 0° C., then stirred overnight at room temperature. The resulting mixture was concentrated under reduced pressure. The residue was purified by silica gel column chromatography, eluting with PE/EtOAc (1:1) to obtain 2-(4-nitrophenoxy)ethyl 4-methylbenzenesulfonate (3.1 g, 84%) as a yellow solid. LCMS (ESI, m/z): 338.1 [M+H]⁺.

### Step B

A solution of 2-(4-nitrophenoxy)ethyl 4-methylbenzenesulfonate (500 mg, 1.48 mmol, 1.0 equiv), 5-fluoro-2-[(oxan-4-ylsulfanyl)methyl]-7-(piperidin-4-ylmethoxy)-3H-quinazolin-4-one (724 mg, 1.77 mmol, 1.2 equiv), DIEA (574 mg, 4.44 mmol, 3.0 equiv) and KI (123 mg, 0.74 mmol, 0.5 equiv) in ACN (5 mL) was stirred for 3 hours at 60° C. The resulting mixture was concentrated under vacuum. The crude product was re-crystallized from DMSO/water (1:1, 30 mL) to afford 5-fluoro-7-((1-(2-(4-nitrophenoxy)ethyl)piperidin-4-yl)methoxy)-2-(((tetrahydro-2H-pyran-4-yl)thio)methyl)quinazolin-4(3H)-one (340 mg, 40%) as a white solid. LCMS (ESI, m/z): 573.2 [M+H]⁺.

### Step C

A solution of 5-fluoro-7-((1-(2-(4-nitrophenoxy)ethyl)piperidin-4-yl)methoxy)-2-(((tetrahydro-2H-pyran-4-yl)thio)methyl)quinazolin-4(3H)-one (632 mg, 1.10 mmol, 1.0 equiv) and Na₂S (596 mg, 22.1 mmol, 20 equiv) in EtOH (10 mL) and water (3 mL) was stirred for 6 hours at 80° C. The solution was diluted with water and extracted with EtOAc (30 mL). The combined organic layers were concentrated. The residue was purified by reverse phase flash chromatography with the following conditions: column, C18 silica gel; mobile phase, MeCN in water (10 mmol/L NH₄HCO₃), 10% to 50% gradient in 10 min; detector, UV 254 nm to afford 7-((1-(2-(4-aminophenoxy)ethyl)piperidin-4-yl)methoxy)-5-fluoro-2-(((tetrahydro-2H-pyran-4-yl)thio)methyl)quinazolin-4(3H)-one (179 mg, 30%) as an off-white solid. LCMS (ESI, m/z): 543.25 [M+H]⁺.

### Step D

A solution of 7-((1-(2-(4-aminophenoxy)ethyl)piperidin-4-yl)methoxy)-5-fluoro-2-(((tetrahydro-2H-pyran-4-yl)thio)methyl)quinazolin-4(3H)-one (158 mg, 0.29 mmol, 1.0 equiv), 3-bromopiperidine-2,6-dione (61.5 mg, 0.32 mmol, 1.1 equiv), DIEA (75.3 mg, 0.58 mmol, 2.0 equiv) and NMP (2 mL) was stirred overnight at 80° C. The residue was purified by reverse phase flash chromatography with the following conditions: column, C18 silica gel; mobile phase, MeCN in water (10 mmol/L NH₄HCO₃), 10% to 70% gradient in 10 min; detector, UV 254 nm. The crude product (128 mg) was purified by prep-HPLC with the following conditions (Column: Xselect CSH F-Phenyl OBD column, 19*250 mm, 5 μm; Mobile Phase A: water (0.1% FA), Mobile Phase B: ACN; Flow rate: 20 mL/min; Gradient: 15% B to 20% B in 8 min, 20% B; Wave Length: 254 nm; RT1 (min): 8) to afford 3-((4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethoxy)phenyl)amino)piperidine-2,6-dione (23 mg, 12%) as a white solid. LCMS (ES, m/z): 654.25 [M+H]⁺. ¹H NMR (400 MHz, DMSO-d₆) δ 12.08 (br, 1H), 10.74 (s, 1H), 6.92-6.83 (m, 2H), 6.73 (d, J=8.7 Hz, 2H), 6.63 (d, J=8.6 Hz, 2H), 5.41 (d, J=7.3 Hz, 1H), 4.30-4.12 (m, 1H), 4.00-3.92 (m, 4H), 3.86-3.78 (m, 2H), 3.63 (s, 2H), 3.40-3.39 (m, 2H), 3.10-3.01 (m, 1H), 3.00-2.90 (m, 2H), 2.78-2.68 (m, 1H), 2.66-2.56 (m, 3H), 2.15-1.99 (m, 3H), 1.95-1.81 (m, 3H), 1.80-1.69 (m, 3H), 1.54-1.40 (m, 2H), 1.39-1.20 (m, 2H).

Examples 59-78 were synthesized according to the procedure described for the synthesis 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1l-yl)ethyl)piperazin-1-yl)phenyl)amino)piperidine-2,6-dione (Example 48) using appropriate building blocks and modified reaction conditions (such as reagents, reagent ratio, temperature, and reaction time) and purification conditions as needed.

| Example | Structure | Analytical Data |
|---|---|---|
| 59 | 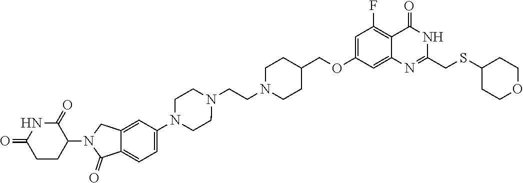 [SMILES: O=C1CCC(N2Cc3cc(N4CCN(CCN5CCC(COc6cc(F)c7c(=O)[nH]c(CSC8CCOCC8)nc7c6)CC5)CC4)ccc3C2=O)C(=O)N1] 3-(5-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1-yl)-1- oxoisoindolin-2-yl)piperidine-2, 6-dione | LCMS (ESI, m/z): 762.35 [M + H]⁺; 1H NMR (300 MHz, DMSO-d6) δ 12.12 (s, 1H), 10.92 (s, 1H), 7.52 (d, J = 9.1 Hz, 1H), 7.05 (d, J = 7.4 Hz, 2H), 6.93 − 6.82 (m, 2H), 5.03 (dd, J = 13.2, 5.0 Hz, 1H), 4.39-4.14 (m, 2H), 3.97 (d, J = 5.9 Hz, 2H), 3.86- 3.76 (m, 2H), 3.62 (s, 2H), 3.31-3.22 |
| | | (m, 6H), 3.11-2.99 |
| | | (m, 1H), 2.96-2.87 |
| | | (m, 3H), 2.64-2.53 |
| | | (m, 5H), 2.47 − |
| | | 2.28 (m, 3H), 2.03 − |
| | | 1.82 (m, 5H), |
| | | 1.80-1.65 (m, 3H), |
| | | 1.54 − 1.24 (m, 6H). |
| 60 | 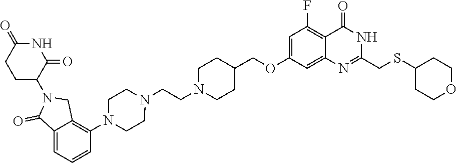 [SMILES: O=C1CCC(N2Cc3c(cccc3N3CCN(CCN4CCC(COc5cc(F)c6c(=O)[nH]c(CSC7CCOCC7)nc6c5)CC4)CC3)C2=O)C(=O)N1] 3-(4-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1-yl)-1- oxoisoindolin-2-yl)piperidine-2,6-dione | LCMS (ESI, m/z): 762.30 [M + H] ⁺; 1H NMR (400 MHz, DMSO-d6) δ 12.11 (s, 1H), 10.98 (s, 1H), 7.44 (t, J = 7.7 Hz, 1H), 7.31 (d, J = 7.5 Hz, 1H), 7.16 (d, J = 8.0 Hz, 1H), 6.92 − 6.83 (m, 2H), 5.11 (dd, J = 13.2, 5.2 Hz, 1H), 4.45 − 4.25 (m, 2H), 3.97 (d, J = |
| | | 5.8 Hz, 2H), |
| | | 3.85 − 3.76 (m, |
| | | 2H), 3.62 (s, 2H), |
| | | 3.36 − 3.31 (m, |
| | | 2H), 3.11 − 3.01 |
| | | (m, 5H), 2.96 − |
| | | 2.88 (m, 4H), 2.64 − |
| | | 2.53 (m, 6H), |
| | | 2.50 − 2.43 (m, |
| | | 3H), 2.03 − 1.85 |
| | | (m, 5H), 1.74-1.69 |
| | | (m, 3H), 1.52 − |
| | | 1.38 (m, 2H), 1.34 − |
| | | 1.26 (m, 2H). |
| 61 | 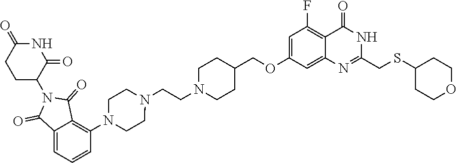 [SMILES: O=C1CCC(N2C(=O)c3cccc(N4CCN(CCN5CCC(COc6cc(F)c7c(=O)[nH]c(CSC8CCOCC8)nc7c6)CC5)CC4)c3C2=O)C(=O)N1] 2-(2,6-dioxopiperidin-3-yl)-4-(4-(2-(4-(((5-fluoro-4-oxo-2- (((tetrahydro-2H-pyran-4-yl)thio)methyl)-3,4- dihydroquinazolin-7-yl)oxy)methyl)piperidin-1- yl)ethyl)piperazin-1-yl)isoindoline-1,3-dione | LCMS (ESI, m/z): 776.40 [M + H] ⁺; 1H NMR (300 MHz, DMSO-d6) δ 12.13 (s, 1H), 11.07 (s, 1H), 7.70 (dd, J = 8.4, 7.2 Hz, 1H), 7.35 (dd, J = 7.8, 5.8 Hz, 2H), 6.93 − 6.82 (m, 2H), 5.09 (dd, J = 12.7, 5.4 Hz, 1H), 3.98 (d, J = 5.8 Hz, 2H), 3.87 − 3.76 (m, 3H), |
| | | 3.62 (s, 2H), 3.30- |
| | | 3.25 (m, 6H) 3.13- |
| | | 3.00 (m, 1H), 2.99- |
| | | 2.80 (m, 3H), |
| | | 2.66-2.55 (m, 6H), |
| | | 2.48-2.40 (m, 3H), |
| | | 2.11 − 1.84 (m, |
| | | 5H), 1.76-1.69 (m, |
| | | 3H), 1.55 −1.20 (m, 4H) |
| 62 | 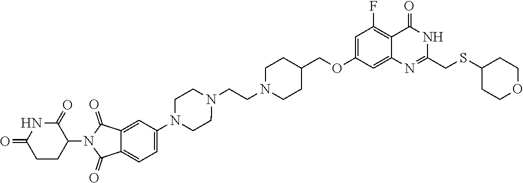 [SMILES: O=C1CCC(N2C(=O)c3ccc(N4CCN(CCN5CCC(COc6cc(F)c7c(=O)[nH]c(CSC8CCOCC8)nc7c6)CC5)CC4)cc3C2=O)C(=O)N1] 2-(2,6-dioxopiperidin-3-yl)-5-(4-(2-(4-(((5-fluoro-4-oxo-2- (((tetrahydro-2H-pyran-4-yl)thio)methyl)-3,4- dihydroquinazolin-7-yl)oxy)methyl)piperidin-1- yl)ethyl)piperazin-1-yl)isoindoline-1,3-dione | LCMS (ESI, m/z): 776.40[M + H] ⁺; ¹H NMR (400 MHz, DMSO-d₆) δ 12.06 (s, 1H), 11.20 − 10.70 (m, 1H), 7.68 (d, J = 7.2 Hz, 1H), 7.33 (s, 1H), 7.25 (d, J = 8.8 Hz, 1H), 6.88-6.82 (m, 2H), 5.15 − 5.00(m, 1H), 4.01 − 3.93 (m, 1H), 3.85 − 3.76 (m, 1H), 3.64 − 3.60 (m, 1H), 3.37 − 3.31 (m, |
| | | 3H), 3.28 − 3.19 |
| | | (m, 2H), 3.18 − |
| | | 3.15(m, 1H), 3.11 − |
| | | 2.99 (m, 2H), |
| | | 2.97 − 2.78 (m, |
| | | 2H), 2.71 − 2.53 |
| | | (m, 5H), 2.47 − |
| | | 2.44 (m, 3H), 2.15 − |
| | | 1.82 (m, 7H), |
| | | 1.79 − 1.66 (m, |
| | | 4H), 1.51 − 1.38 |
| | | (m, 3H). 1.37 − |
| | | 1.19 (m, 3H) |
| 63 | 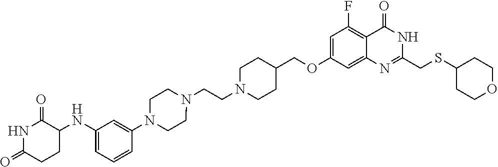 [SMILES: O=C1CCC(Nc2cccc(N3CCN(CCN4CCC(COc5cc(F)c6c(=O)[nH]c(CSC7CCOCC7)nc6c5)CC4)CC3)c2)C(=O)N1] 3-((3-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1- yl)phenyl)amino)piperidine-2,6-dione | LCMS (ESI, m/z):722.30 [M + H] ⁺; 1H NMR (400 MHz, DMSO-d₆) δ 12.11 (s, 1H), 10.75 (s, 1H), 6.95 − 6.84 (m, 3H), 6.26 (S, ^{1H),} 6.20-6.09 (m, 2H), 5.61 (d, J = 7.6 Hz, 1H), 4.34- 4.26 (m, 1H), 3.98 (d, J = 5.9 Hz, 2H), 3.86-3.79 (m, 2H), 3.62 (s, 2H), |
| | | 3.37-3.48(m, 2H), |
| | | 3.31-3.28(m, 2H), |
| | | 3.12 − 3.01 (m, |
| | | 5H), 2.92 (d, J = |
| | | 10.8 Hz, 2H), |
| | | 2.81-2.67 (m, |
| | | 1H), 2.62-2.57 (m, |
| | | 1H), 2.56-2.51 (m, |
| | | 2H), 2.52-2.44 (m, |
| | | 4H), 2.14 − 2.06 |
| | | (m, 1H), 2.00 − |
| | | 1.78 (m, 5H), 1.74- |
| | | 1.69 (m, 3H), |
| | | 1.52-1.39 (m, 2H), |
| | | 1.37-1.23 (m, 2H). |
| 64 | 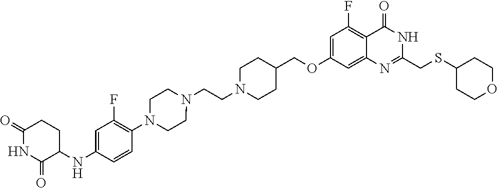 [SMILES: O=C1CCC(Nc2ccc(N3CCN(CCN4CCC(COc5cc(F)c6c(=O)[nH]c(CSC7CCOCC7)nc6c5)CC4)CC3)c(F)c2)C(=O)N1] 3-((3-fluoro-4-(4-(2-(4-(((5-fluoro-4-oxo-2-(((tetrahydro-2H- pyran-4-yl)thio)methyl)-3,4-dihydroquinazolin-7- yl)oxy)methyl)piperidin-1-yl)ethyl)piperazin-1- yl)phenyl)amino)piperidine-2,6-dione | LCMS (ESI, m/z): 740.30 [M + H] ⁺; 1H NMR (400 MHz, DMSO-d6) δ 12.11 (br s, 1H), 10.78 (s, 1H), 6.89-6.79 (m, 3H), 6.51 (dd, J = 15.0, 2.4 Hz, 1H), 6.45- 6.38 (m, 1H), 5.80 (d, J = 8.0 Hz, 1H), 4.31 − 4.20 (m, 1H), 3.97 (d, J = 5.8 Hz, 2H), 3.86 − 3.78 (m, 2H), 3.61 (s, 2H), 3.45-3.39 (m, 2H), |
| | | 3.30-3.23 (m, 2H), |
| | | 3.09-3.00 (m, 1H), |
| | | 2.92 (d, J = 10.4 |
| | | Hz, 2H), 2.84 − |
| | | 2.79 (m, 4H), |
| | | 2.78-2.66 (m, 1H), |
| | | 2.61-2.56 (m, 1H) |
| | | 2.55-2.53 (m, 3H), |
| | | 2.48-2.44 (m, 3H), |
| | | 2.13-2.04 (m, |
| | | 1H), 1.99-1.85 (m, |
| | | 5H), 1.78-1.71 (m, |
| | | 3H), 1.51-1.37 (m, |
| | | 2H), 1.36-1.22 (m, 2H). |
| 65 | 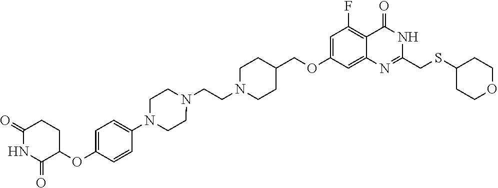 [SMILES: O=C1CCC(Oc2ccc(N3CCN(CCN4CCC(COc5cc(F)c6c(=O)[nH]c(CSC7CCOCC7)nc6c5)CC4)CC3)cc2)C(=O)N1] 3-(4-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1- yl)phenoxy)piperidine-2,6-dione | LCMS (ESI, m/z): 723.50 [M + H] ⁺; 1H NMR (400 MHz, DMSO-d6) δ 12.16 (s, 1H), 10.88 (s, 1H), 6.94 − 6.83 (m, 6H), 5.01 (dd, J = 10.4, 5.2 Hz, 1H), 3.97 (d, J = 5.7 Hz, 2H), 3.86 − 3.77 (m, 2H), 3.62 (s, 2H), 3.79-3.33 (m, 3H), 3.31-3.29 (m, 1H), 3.09- 3.05(m, 1H), 3.02- 2.99 (m, 4H), 2.92 |
| | | (d, J = 10.9 Hz, |
| | | 2H), 2.72 − 2.58 |
| | | (m, 2H), 2.57 − |
| | | 2.55 (m, 2H), 2.45 |
| | | (s, 4H), 2.22-2.02 |
| | | (m, 2H), 1.99-1.85 |
| | | (m, 4H), 1.73 (d, J = |
| | | 10.8 Hz, 3H), |
| | | 1.52 − 1.38 (m, |
| | | 2H), 1.36 − 1.21 (m, 2H) |
| 66 | 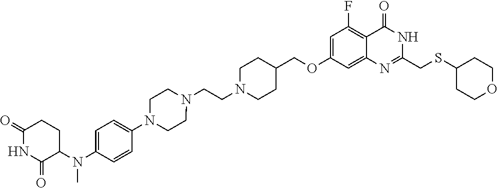 [SMILES: CN(c1ccc(N2CCN(CCN3CCC(COc4cc(F)c5c(=O)[nH]c(CSC6CCOCC6)nc5c4)CC3)CC2)cc1)C1CCC(=O)NC1=O] 3-((4-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1- yl)phenyl)(methyl)amino)piperidine-2,6-dione | LCMS (ESI, m/z): 736.30 [M + H] ⁺; 1H NMR (400 MHz, DMSO-d6) δ 12.12 (s, 1H), 10.72 (s, 1H), 6.90-6.85(m, 2H), 6.82(d, J = 9.2 Hz, 2H), 6.75(d, J = 8.4 Hz, 2H), 4.72 (dd, J = 12.7, 4.9 Hz, 1H), 3.97 (d, J = 5.9 Hz, 2H), 3.89- 3.74 (m, 2H), 3.62 (s, 2H), 3.08-3.01 (m, 1H), 2.99- 2.88 (m, |
| | | 6H), 2.86 − 2.76 |
| | | (m, 1H), 2.68 (s, |
| | | 3H), 2.45-2.38 (m, |
| | | 4H), 2.29 − 2.18 |
| | | (m, 1H), 1.95-1.80 |
| | | (m, 5H), 1.78-1.71 |
| | | (m, 3H), 1.51 − |
| | | 1.38 (m, 2H), 1.38 − |
| | | 1.20 (m, 3H). |
| 67 | 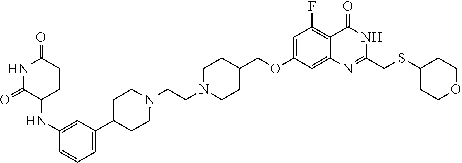 [SMILES: O=C1CCC(Nc2cccc(C3CCN(CCN4CCC(COc5cc(F)c6c(=O)[nH]c(CSC7CCOCC7)nc6c5)CC4)CC3)c2)C(=O)N1] 3-((3-(1-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperidin-4- yl)phenyl)amino)piperidine-2,6-dione | LCMS (ESI, m/z):721.30 [M + H] ⁺; 1H NMR (400 MHz, DMSO-d6) δ 12.16 (s, 1H), 10.76 (s, 1H), 8.22 (s, 1H), 6.99 (t, J = 7.8 Hz, 1H), 6.92 − 6.84 (m, 2H), 6.55 (d, J = 2.3 Hz, 1H), 6.52 − 6.42 (m, 2H), 5.72 (d, J = 7.6 Hz, 1H), 4.38 − 4.30 (m, 1H), |
| | | 3.98 (d, J = 5.9 |
| | | Hz, 2H), 3.85 − |
| | | 3.79 (m, 2H), 3.62 |
| | | (s, 2H), 3.09 − |
| | | 3.03 (m, 2H), 2.99 − |
| | | 2.91 (m, 3H), |
| | | 2.80 − 2.72 (m, |
| | | 1H), 2.65 −2.55 (m, |
| | | 1H), 2.51 −2.41 (m, |
| | | 3H), 2.34 −2.25(m, |
| | | 2H), 2.13 − 1.93 |
| | | (m, 6H), 1.88 − |
| | | 1.82(m, 3H), 1.74 − |
| | | 1.59 (m, 6H), |
| | | 1.50 − 1.41 (m, |
| | | 2H), 1.31 − 1.15 (m, 4H). |
| 68 | 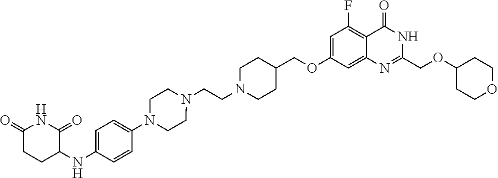 [SMILES: O=C1CCC(Nc2ccc(N3CCN(CCN4CCC(COc5cc(F)c6c(=O)[nH]c(COC7CCOCC7)nc6c5)CC4)CC3)cc2)C(=O)N1] 3-((4-(4-(2-(4-(((5-fluoro-4-oxo-2-(((tetrahydro-2H-pyran-4- yl)oxy)methyl)-3,4-dihydroquinazolin-7- yl)oxy)methyl)piperidin-1-yl)ethyl)piperazin-1- yl)phenyl)amino)piperidine-2,6-dione | LCMS (ESI, m/z):706.30 [M + H] ⁺; 1H NMR (400 MHz, DMSO-d6) δ 12.00 (s, 1H), 10.75 (s, 1H), 6.97 − 6.82 (m, 2H), 6.75 (d, J = 8.8 Hz, 2H), 6.61 (d, J = 8.9 Hz, 2H), 5.36 (d, J = 7.2 Hz, 1H), 4.37 (s, 2H), 4.21 − 4.15 (m, 1H), 3.98 (d, J = 4.0 Hz, 2H), 3.87 − 3.78 |
| | | (m, 2H), 3.71 − |
| | | 3.57 (m, 1H), 3.34 |
| | | (s, 1H), 3.32-3.29 |
| | | (m, 1H), 2.92 − |
| | | 2.91 (m, 6H), 2.73 − |
| | | 2.69 (m, 1H), |
| | | 2.68 − 2.67 (m, |
| | | 1H), 2.53 − 2.52 |
| | | (m, 4H), 2.51-2.44 |
| | | (m, 4H), 2.11 − |
| | | 2.09 (m, 1H), 2.10 − |
| | | 1.84 (m, 5H), |
| | | 1.81 − 1.67 (m, |
| | | 3H), 1.54 − 1.41 |
| | | (m, 2H), 1.36 − |
| | | 1.21 (m, 2H). |
| 69 | 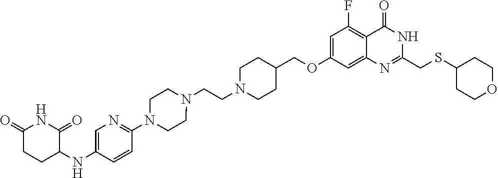 [SMILES: O=C1CCC(Nc2ccc(N3CCN(CCN4CCC(COc5cc(F)c6c(=O)[nH]c(CSC7CCOCC7)nc6c5)CC4)CC3)nc2)C(=O)N1] 3-((6-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1-yl)pyridin-3- yl)amino)piperidine-2,6-dione | LCMS (ESI, m/z): 723.30 [M + H] ⁺; 1H NMR (300 MHz, DMSO-d6) δ 12.01 (s, 1H), 10.74 (s, 1H), 7.70 (d, J = 2.9 Hz, 1H), 7.05 (dd, J = 9.1, 3.0 Hz, 1H), 6.87 − 6.81 (m, 2H), 6.68 (d, J = 8.9 Hz, 1H), 5.38 (d, J = 7.5 Hz, 1H), 4.27 − 4.13 (m, 1H), 3.97 (d, J = 5.8 Hz, |
| | | 2H), 3.88-3.75 (m, |
| | | 2H), 3.62 (s, 2H), |
| | | 3.55 − 3.34 (m, |
| | | 4H), 3.25 − 3.15 |
| | | (m, 4H), 3.13 − |
| | | 3.00 (m, 1H), 2.92 |
| | | (d, J = 10.7 Hz, |
| | | 2H), 2.75 − 2.56 |
| | | (m, 2H), 2.54 − |
| | | 2.50(m, 2H), 2.48 − |
| | | 2.41 (m, 4H), |
| | | 2.18 − 2.06 (m, |
| | | 1H), 2.02 − 1.89 |
| | | (m, 5H), 1.87 (m, |
| | | 3H), 1.55 − 1.36 |
| | | (m, 2H), 1.35 − |
| | | 1.21 (m, 2H). |
| 70 | 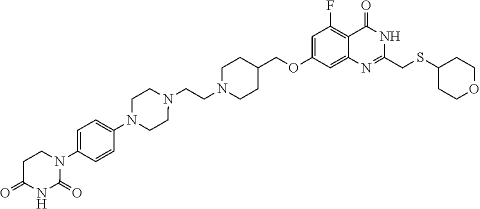 [SMILES: O=C1CCN(c2ccc(N3CCN(CCN4CCC(COc5cc(F)c6c(=O)[nH]c(CSC7CCOCC7)nc6c5)CC4)CC3)cc2)C(=O)N1] 1-(4-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1- yl)phenyl)dihydropyrimidine-2,4(1H,3H)-dione | LCMS (ESI, m/z): 708.35 [M + H] ⁺; 1H NMR (400 MHz, Chloroform-d) δ 9.68 (s, 1H), 7.47 (s, 1H), 7.24 − 7.16 (m, 2H), 6.98 − 6.89 (m, 2H), 6.86 (d, J = 2.4 Hz, 1H), 6.71 (dd, J = 11.9, 2.4 Hz, 1H), 4.02-3.92 (m, 4H), 3.84 (t, J = 6.7 Hz, 2H), 3.76 (s, 3H), 3.45 − 3.35 (m, 2H), 3.27-3.21 (m, 4H), 3.18-3.02 (m, 4H), |
| | | 2.95 − 2.87 (m, |
| | | 1H), 2.86-2.80 (m, |
| | | 3H), 2.79-2.71 (m, |
| | | 5H), 2.60-2.45 |
| | | (m, 2H) 2.12- |
| | | 2.01 (m, 4H), 1.91 − |
| | | 1.86 (m, 2H), |
| | | 1.75 − 1.61 (m, 2H). |
| 71 | 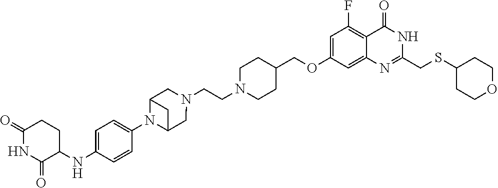 [SMILES: O=C1CCC(Nc2ccc(N3C4CC3CN(CCN3CCC(COc5cc(F)c6c(=O)[nH]c(CSC7CCOCC7)nc6c5)CC3)C4)cc2)C(=O)N1] 3-((4-(3-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)-3,6- diazabicyclo[3.1.1]heptan-6-yl)phenyl)amino)piperidine-2,6-dione | LCMS (ESI, m/z): 734.30[M + H] ⁺; 1H NMR (300 MHz, DMSO-d6) δ 10.72 (s, 1H), 7.03 − 6.70 (m, 2H), 6.60 (d, J = 8.3 Hz, 2H), 6.27 (d, J = 8.3 Hz, 2H), 5.13 (d, J = 6.6 Hz, 1H), 4.10 (d, J = 5.8 Hz, 3H), 3.93 (d, J = 6.0 Hz, 2H), 3.81 (d, J = 11.5 Hz, 2H), 3.61 (s, 3H), 3.33-3.31(m, 2H), |
| | | 3.29-3.27 (m, |
| | | 1H) 3.15-3.00 |
| | | (m, 3H), 2.80-2.59 |
| | | (m, 5H), 2.58 − |
| | | 2.50(m, 1H), 2.43 − |
| | | 2.33 (m, 3H), |
| | | 2.28-2.21 (m, 2H), |
| | | 2.17-2.06 (m, 1H), |
| | | 1.90-1.79 (m, 5H), |
| | | 1.72-1.60 (m, 3H), |
| | | 1.51 − 1.34 (m, |
| | | 2H), 1.30-1.15 (m, 2H). |
| 72 | 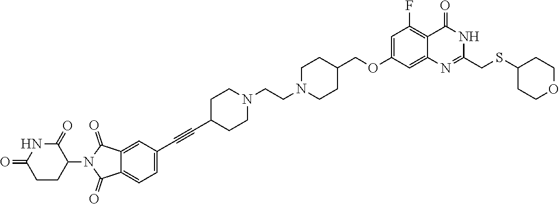 [SMILES: O=C1CCC(N2C(=O)c3ccc(C#CC4CCN(CCN5CCC(COc6cc(F)c7c(=O)[nH]c(CSC8CCOCC8)nc7c6)CC5)CC4)cc3C2=O)C(=O)N1] 2-(2,6-dioxopiperidin-3-yl)-5-((1-(2-(4-(((5-fluoro-4-oxo-2- (((tetrahydro-2H-pyran-4-yl)thio)methyl)-3,4- dihydroquinazolin-7-yl)oxy)methyl)piperidin-1- yl)ethyl)piperidin-4-yl)ethynyl)isoindoline-1,3-dione | LCMS (ESI, m/z): 799.40[M + H] ⁺; 1H NMR (400 MHz, Methanol- d4) δ 7.93 − 7.85 (m, 3H), 6.98 (s, 1H), 6.86 (dd, J = 12.4, 2.0 Hz, 1H), 5.17 (dd, J = 12.5, 5.4 Hz, 1H), 4.09 (d, J = 5.7 Hz, 2H), 3.96 − 3.90 (m, 2H), 3.74 − 3.60 (m, 4H), 3.52 − 3.43 (m, 4H), 3.43 − 3.34 (m, 5H), 3.14 − 3.00 (m, 5H), 2.95 − |
| | | 2.83 (m, 1H), 2.82 − |
| | | 2.67 (m, 2H), |
| | | 2.28 − 2.10 (m, |
| | | 6H), 2.10 − 1.92 |
| | | (m, 4H), 1.86 − |
| | | 1.72 (m, 2H), 1.67 − |
| | | 1.53 (m, 2H). |
| 73 | 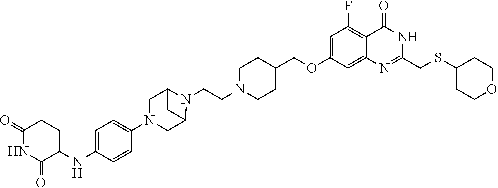 [SMILES: O=C1CCC(Nc2ccc(N3CC4CC(C3)N4CCN3CCC(COc4cc(F)c5c(=O)[nH]c(CSC6CCOCC6)nc5c4)CC3)cc2)C(=O)N1] 3-((4-(6-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)-3,6- diazabicyclo[3.1.1]heptan-3-yl)phenyl)amino)piperidine-2,6-dione. | LCMS (ESI, m/z):734.30 [M + H] ⁺; 1H NMR (300 MHz, DMSO-d6) δ 10.73 (s, 1H), 6.98 − 6.85 (m, 2H), 6.81 − 6.62 (m, 2H), 6.62 − 6.35 (m, 2H), 5.06 (d, J = 7.0 Hz, 1H), 4.20 − 4.04 (m, 1H), 4.00 − 3.88 (m, 2H), 3.86 − 3.75 (m, 2H), 3.70 − 3.59 (m, 3H), 3.58 − 3.50 |
| | | (m, 2H), 3.26 − |
| | | 3.13 (m, 3H), 3.10 − |
| | | 3.00 (m, 1H), |
| | | 2.90 − 2.75 (m, |
| | | 2H), 2.75 − 2.59 |
| | | (m, 2H), 2.50 − |
| | | 2.35 (m, 4H), 2.22 − |
| | | 2.00 (m, 2H), |
| | | 1.99 − 1.80 (m, |
| | | 4H), 1.77 − 1.60 |
| | | (m, 3H), 1.59 − |
| | | 1.39 (m, 3H), |
| | | 1.38-1.20(m, 5H). |
| 74 | 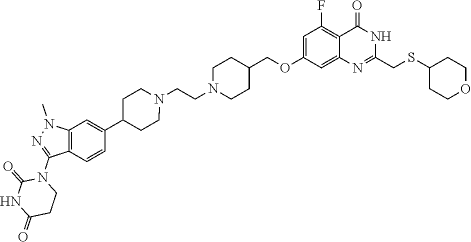 [SMILES: Cn1nc(N2CCC(=O)NC2=O)c2ccc(C3CCN(CCN4CCC(COc5cc(F)c6c(=O)[nH]c(CSC7CCOCC7)nc6c5)CC4)CC3)cc21] 1-(6-(1-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperidin-4-yl)-1-methyl- 1H-indazol-3-yl)dihydropyrimidine-2,4(1H,3H)-dione | LCMS (ESI, m/z): 761.40 [M + H] ⁺; 1H NMR (300 MHz, DMSO-d6) δ 12.18 (s, 1H), 10.55 (s, 1H), 7.55 (d, J = 8.5 Hz, 1H), 7.45 (s, 1H), 7.09 − 6.99 (m, 1H), 6.94 − 6.83 (m, 2H), 4.10-3.83 (m, 9H), 3.62 (s, 2H), 3.37 − 3.35 (m, 1H), 3.30 − 3.27 (m, 1H), 3.12 − 2.97 (m, 3H), 2.96 − 2.87 (m, 2H) 2.78 − 2.74 (m, 2H), 2.70-2.55 (m, 1H), |
| | | 2.45 (s, 4H), 2.12 − |
| | | 1.89 (m, 7H), |
| | | 1.86 − 1.71 (m, |
| | | 6H), 1.52 − 1.46 |
| | | (m, 2H), 1.47 − |
| | | 1.21 (m, 2H). |
| 75 | 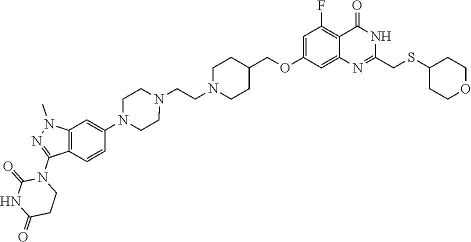 [SMILES: Cn1nc(N2CCC(=O)NC2=O)c2ccc(N3CCN(CCN4CCC(COc5cc(F)c6c(=O)[nH]c(CSC7CCOCC7)nc6c5)CC4)CC3)cc21] 1-(6-(4-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperazin-1-yl)-1-methyl- 1H-indazol-3-yl)dihydropyrimidine-2,4(1H,3H)-dione | LCMS (ESI, m/z): 762.35[M + H]⁺; 1H NMR (300 MHz, DMSO-d6) δ 12.18 (s, 1H), 10.52 (s, 1H), 7.45 (d, J = 9.0 Hz, 1H), 6.97 − 6.85 (m, 3H), 6.82 (d, J = 1.9 Hz, 1H), 3.98 (d, J = 5.8 Hz, 2H), 3.92- 3.76 (m, 7H), 3.62 (s, 2H), 3.30-3.15 (m, 5H), 3.12 − 2.98 (m, 1H), 2.94 (d, J = 10.9 Hz, 2H), 2.74 (t, J = 6.7 Hz, 2H), 2.59 (d, J = 4.8 Hz, |
| | | 4H), 2.51-2.43 (m, |
| | | 4H), 2.04 − 1.84 |
| | | (m, 4H), 1.80-1.65 |
| | | (m, 3H), 1.49 − |
| | | 1.41(m, 2H), 1.40 − |
| | | 1.27 (m, 3H). |
| 76 | 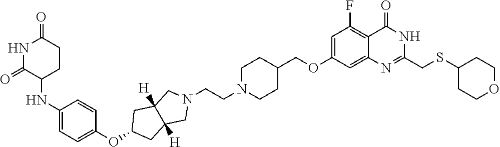 [SMILES: O=C1CCC(Nc2ccc(O[C@@H]3C[C@@H]4CN(CCN5CCC(COc6cc(F)c7c(=O)[nH]c(CSC8CCOCC8)nc7c6)CC5)C[C@@H]4C3)cc2)C(=O)N1] 3-((4-(((3aR,51,6aS)-2-(2-(4-(((5-fluoro-4-oxo-2-(((tetrahydro- 2H-pyran-4-yl)thio)methyl)-3,4-dihydroquinazolin-7- yl)oxy)methyl)piperidin-1- yl)ethyl)octahydrocyclopenta[c]pyrrol-5- yl)oxy)phenyl)amino)piperidine-2,6-dione | LCMS (ESI, m/z): 763.55 [M + H] ⁺; 1H NMR (300 MHz, DMSO-d6) δ 11.01-10.09 (m,1H), 6.86 (s, 1H), 6.83 (d, J = 2.3 Hz, 1H), 6.71 (d, J = 8.9 Hz, 2H), 6.63 (d, J = 8.9 Hz, 2H), 5.40 (d, J = 7.2 Hz, 1H), 4.54 − 4.44 (m, 1H), 4.25-4.13 (m, 1H), 3.97 (d, J = 5.9 Hz, 2H), |
| | | 3.75-3.88 (m, 2H), |
| | | 3.61 (s, 2H), 3.45- |
| | | 3.35 (m, 2H), |
| | | 3.11-3.00 (m, 1H), |
| | | 2.93-2.85 (m, 2H), |
| | | 2.80-2.55 (m, 3H), |
| | | 2.49-2.38 (m, 7H), |
| | | 2.19-2.05 (m, 3H), |
| | | 2.01-1.81 (m, |
| | | 5H), 1.80-1.65 (m, |
| | | 3H), 1.64-1.55 (m, |
| | | 5H), 1.38-1.21 (m, 3H). |
| 77 | 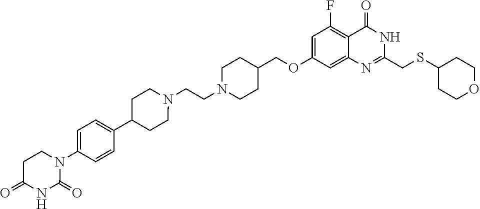 [SMILES: O=C1CCN(c2ccc(C3CCN(CCN4CCC(COc5cc(F)c6c(=O)[nH]c(CSC7CCOCC7)nc6c5)CC4)CC3)cc2)C(=O)N1] 1-(4-(1-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperidin-4- yl)phenyl)dihydropyrimidine-2,4(1H,3H)-dione | LCMS (ESI, m/z): 707.25[M + H] ⁺; 1H NMR (400 MHz, DMSO-d6) δ 12.10 (s, 1H), 10.34 (s, 1H), 7.29 − 7.20 (m, 4H), 6.91 − 6.83 (m, 2H), 3.97 (d, J = 5.9 Hz, 2H), 3.86 −3.72 (m, 4H) 3.61 (s, 2H), 3.31 −3.25 (m, 3H), 3.11 − 3.01 (m, 1H), 2.98 (d, J = 10.8 Hz, 2H), 2.91 (d, J = 10.8 Hz, 2H), 2.69 (t, J = |
| | | 6.6 Hz, 2H), |
| | | 2.50 − 2.43 (m, |
| | | 4H), 2.03 − 1.85 |
| | | (m, 6H), 1.78 − |
| | | 1.53 (m, 7H), 1.51 − |
| | | 1.38 (m, 2H), |
| | | 1.34 − 1.22 (m, 2H). |
| 78 | 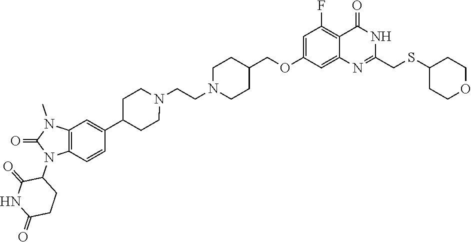 [SMILES: Cn1c(=O)n(C2CCC(=O)NC2=O)c2ccc(C3CCN(CCN4CCC(COc5cc(F)c6c(=O)[nH]c(CSC7CCOCC7)nc6c5)CC4)CC3)cc21] 3-(5-(1-(2-(4-(((5-fluoro-4-oxo-2-(((tetrahydro-2H-pyran-4- yl)thio)methyl)-3,4-dihydroquinazolin-7- yl)oxy)methyl)piperidin-1-yl)ethyl)piperidin-4-yl)-3-methyl- 2-oxo-2,3-dihydro-1H-benzo[d]imidazol-1-yl)piperidine-2,6- dione | LCMS (ESI, m/z): 776.20 [M + H]⁺; ¹H NMR (300 MHz, DMSO-d₆) δ 12.21 (s, 1H), 11.08 (s, 1H), 7.36 − 7.04 (m, 2H), 7.00 − 6.83 (m, 3H), 5.36 (dd, J = 12.7, 5.4 Hz, 1H), 4.10-4.05 (m, 3H), 3.83 − 3.75 (m, 5H), 3.64 − 3.40 (m, 9H), 3.39 − 3.26 (m, 5H), 3.18 − 3.00 (m, 4H), 2.98 − 2.84 (m, 2H), 2.76 − 2.57 (m, 2H), 2.18 − 1.82 (m, 9H), 1.75 − 1.40 (m, 4H). |

### Example 64: Synthesis of 3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione

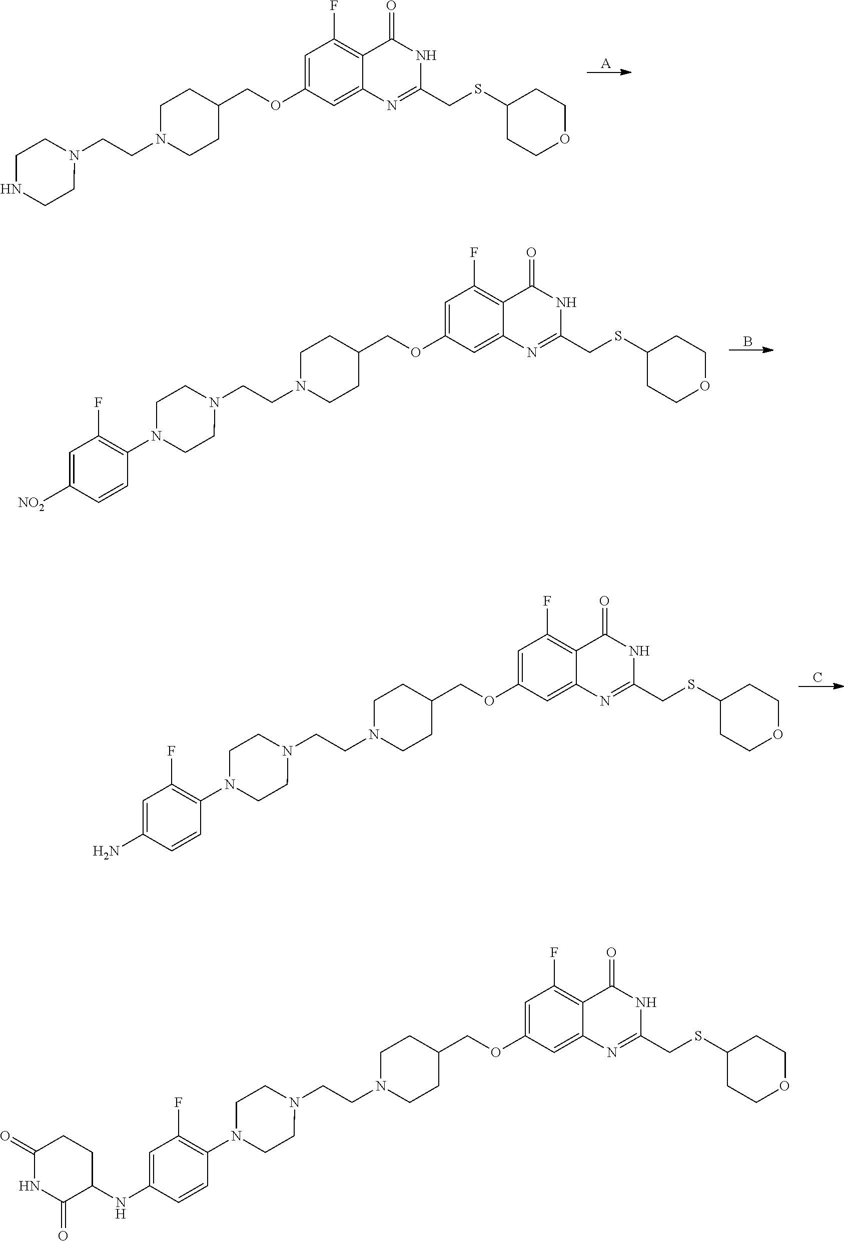

### Step A

A mixture of 5-fluoro-7-((1-(2-(piperazin-1-yl)ethyl)piperidin-4-yl)methoxy)-2-(((tetrahydro-2H-pyran-4-yl)thio)methyl)quinazolin-4(3H)-one (5 g, 9.62 mmol, 1 equiv), 1,2-difluoro-4-nitrobenzene (1.53 g, 9.62 mmol, 1 equiv) and NaHCO₃ (3.23 g, 38.5 mmol, 4 equiv) in ACN (6 mL) was stirred for 3 hours at 90° C. The mixture was concentrated and the residue was purified by silica gel column chromatography, eluting with CH₂Cl₂/MeOH (10:1) to afford 5-fluoro-7-((1-(2-(4-(2-fluoro-4-nitrophenyl)piperazin-1-yl)ethyl)piperidin-4-yl)methoxy)-2-(((tetrahydro-2H-pyran-4-yl)thio)methyl)quinazolin-4(3H)-one (3.2 g, 55%) as a yellow solid. LCMS (ESI, m/z): 657.15 [M−H]⁻.

### Step B

A solution of 5-fluoro-7-((1-(2-(4-(2-fluoro-4-nitrophenyl)piperazin-1-yl)ethyl)piperidin-4-yl)methoxy)-2-(((tetrahydro-2H-pyran-4-yl)thio)methyl)quinazolin-4(3H)-one (3.2 g, 4.9 mmol, 1 equiv), Fe (1.36 g, 24.3 mmol, 5 equiv) and NH₄Cl (0.52 g, 9.72 mmol, 2 equiv) in EtOH (50 mL) and water (5 mL) was stirred for 5 hours at 80° C. The mixture was filtered, and the filter cake was washed with EtOH (5×10 mL). The filtrate was concentrated and the residue was purified by silica gel column chromatography, eluting with CH₂Cl₂/MeOH (8:1) to afford 7-((1-(2-(4-(4-amino-2-fluorophenyl)piperazin-1-yl)ethyl)piperidin-4-yl)methoxy)-5-fluoro-2-(((tetrahydro-2H-pyran-4-yl)thio)methyl)quinazolin-4(3H)-one (2.6 g, 85%) as a white solid. LCMS (ESI, m/z): 627.30 [M−H]⁻.

### Step C

A mixture of 7-((1-(2-(4-(4-amino-2-fluorophenyl)piperazin-1-yl)ethyl)piperidin-4-yl)methoxy)-5-fluoro-2-(((tetrahydro-2H-pyran-4-yl)thio)methyl)quinazolin-4(3H)-one (1.8 g, 2.9 mmol, 1 equiv), 3-bromopiperidine-2,6-dione (3.02 g, 15.7 mmol, 5.5 equiv), and NaHCO₃ (1.92 g, 22.9 mmol, 8 equiv) in ACN (5 mL) was stirred for 4 days at 90° C. The residue was purified directly by silica gel column chromatography, eluting with CH₂Cl₂/EtOH (10:1). The mixture was concentrated and the residue purified by reversed-phase flash chromatography with the following conditions: column, C18 silica gel; mobile phase, MeCN in water (10 mmol/L NH₄HCO₃), 10% to 25% gradient in 15 min; detector, UV 254 nm. This resulted in 3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione (962 mg, 45%) as a white solid. LCMS (ESI, m/z): 740.15 [M+H]⁺. 1H NMR (300 MHz, DMSO-d6) 6 NMR (400 MHz, DMSO-d6) δ 12.11 (br s, 1H), 10.78 (s, 1H), 6.89-6.79 (m, 3H), 6.51 (dd, J=15.0, 2.4 Hz, 1H), 6.45-6.38 (m, 1H), 5.80 (d, J=8.0 Hz, 1H), 4.31-4.20 (m, 1H), 3.97 (d, J=5.8 Hz, 2H), 3.86-3.78 (m, 2H), 3.61 (s, 2H), 3.45-3.39 (m, 2H), 3.30-3.23 (m, 2H), 3.09-3.00 (m, 1H), 2.92 (d, J=10.4 Hz, 2H), 2.84-2.79 (m, 4H), 2.78-2.66 (m, 1H), 2.61-2.56 (m, 1H) 2.55-2.53 (m, 3H), 2.48-2.44 (m, 3H), 2.13-2.04 (m, 1H), 1.99-1.85 (m, 5H), 1.78-1.71 (m, 3H), 1.51-1.37 (m, 2H), 1.36-1.22 (m, 2H).

### Example 64a and 64b: Separation of (R)-3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione and (S)-3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione

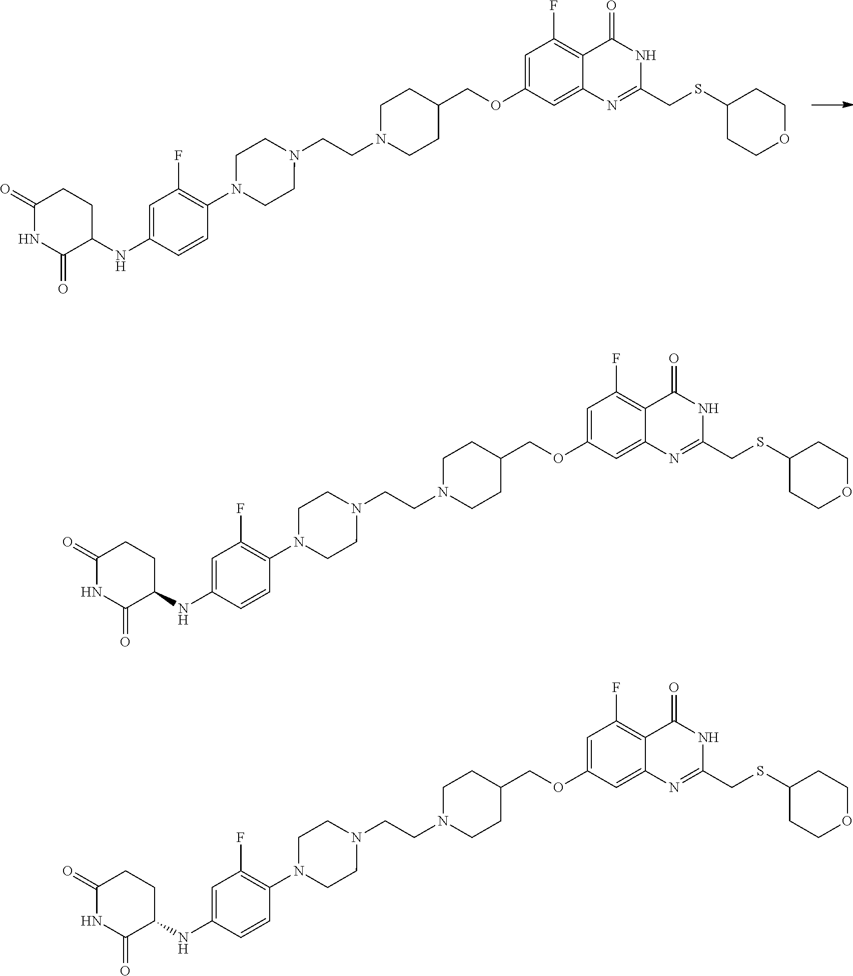 [SMILES: O=C1CCC(Nc2ccc(N3CCN(CCN4CCC(COc5cc(F)c6c(=O)[nH]c(CSC7CCOCC7)nc6c5)CC4)CC3)c(F)c2)C(=O)N1 ; O=C1CC[C@@H](Nc2ccc(N3CCN(CCN4CCC(COc5cc(F)c6c(=O)[nH]c(CSC7CCOCC7)nc6c5)CC4)CC3)c(F)c2)C(=O)N1 ; O=C1CC[C@H](Nc2ccc(N3CCN(CCN4CCC(COc5cc(F)c6c(=O)[nH]c(CSC7CCOCC7)nc6c5)CC4)CC3)c(F)c2)C(=O)N1]

500 mg of the racemic 3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1l-yl)ethyl)piperazin-1-yl)phenyl)amino)piperidine-2,6-dione (Example 64) was separated by chiral prep HPLC with following conditions: (Column: CHIRALPAK IF, 2*25 cm, 5 μm; Mobile Phase A: Hex (0.5% 2M NH₃-MeOH), Mobile Phase B: EtOH:DCM=1:1; Flow rate: 20 mL/min; Gradient: 80% B to 80% B in 32 min; Wave Length: 220/254 nm; RT (min): 8.17 (Example 64a); RT (min): 17.82 (Example 64b); Sample Solvent: EtOH:DCM=1:1-HPLC; Sample concentration: 22.2 mg/mL; Injection Volume: 1.5 mL; Number Of Runs: 15) The fractions were concentrated and then lyophilized to afford the title compounds. Absolute stereochemistry was not assigned.

Example 64a: The first eluting isomer isolated using the above conditions (213 mg) as a white solid. LCMS (ESI, m/z): 740.40 [M+H]⁺. ¹H NMR (400 MHz, DMSO-d₆) δ 12.16 (s, 1H), 10.78 (s, 1H), 6.96-6.76 (m, 3H), 6.51 (dd, J=15.2, 2.5 Hz, 1H), 6.46-6.39 (m, 1H), 5.80 (d, J=7.7 Hz, 1H), 4.30-4.21 (m, 1H), 3.98 (d, J=6.0 Hz, 2H), 3.85-3.79 (m, 2H), 3.62 (s, 2H), 3.36 (d, J=2.3 Hz, 2H), 3.30 (s, 2H), 3.11-3.01 (m, 1H), 2.93 (s, 2H), 2.84 (s, 4H), 2.78-2.69 (m, 1H), 2.59 (d, J=4.2 Hz, 3H), 2.46 (s, 4H), 2.09 (dd, J=12.4, 4.4 Hz, 1H), 1.96 (s, 2H), 1.89 (d, J=13.6 Hz, 3H), 1.74 (d, J=11.7 Hz, 3H), 1.50-1.42 (m, 2H), 1.32-1.24 (m, 2H). Column: CHIRALPAK IF-3; Mobile Phase A: Hex (0.2% DEA):(EtOH:DCM=1:1)=20:80; Flow rate: 1.0 mL/min; Retention time: 1.707 min (faster peak).

Example 64b: The second eluting isomer isolated using the above conditions (209 mg) as white solid. LCMS (ESI, m/z): 740.35 [M+H]⁺. ¹H NMR (400 MHz, DMSO-d₆) δ 12.17 (s, 1H), 10.78 (s, 1H), 6.92-6.79 (m, 3H), 6.51 (dd, J=15.1, 2.5 Hz, 1H), 6.46-6.38 (m, 1H), 5.81 (d, J=7.7 Hz, 1H), 4.31-4.20 (m, 1H), 4.00 (s, 2H), 3.86-3.77 (m, 2H), 3.62 (s, 2H), 3.30-3.20 (m, 2H), 3.11-3.01 (m, 2H), 2.95-2.86 (m, 6H), 2.79-2.65 (m, 2H), 2.66-2.50 (m, 6H), 2.13-2.04 (m, 1H), 2.03-1.70 (m, 7H), 1.50-1.20 (m, 4H). Column: CHIRALPAK IF3; Mobile Phase A: Hex (0.2% DEA):(EtOH:DCM=1:1)=20:80; Flow rate: 1.0 mL/min; Retention time: 4.064 min (slower peak).

### Example 79a and 79b: Synthesis of 2-(2,6-dioxopiperidin-3-yl)-5-((1r,3r)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione and 2-(2,6-dioxopiperidin-3-yl)-5-((1s,3s)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione

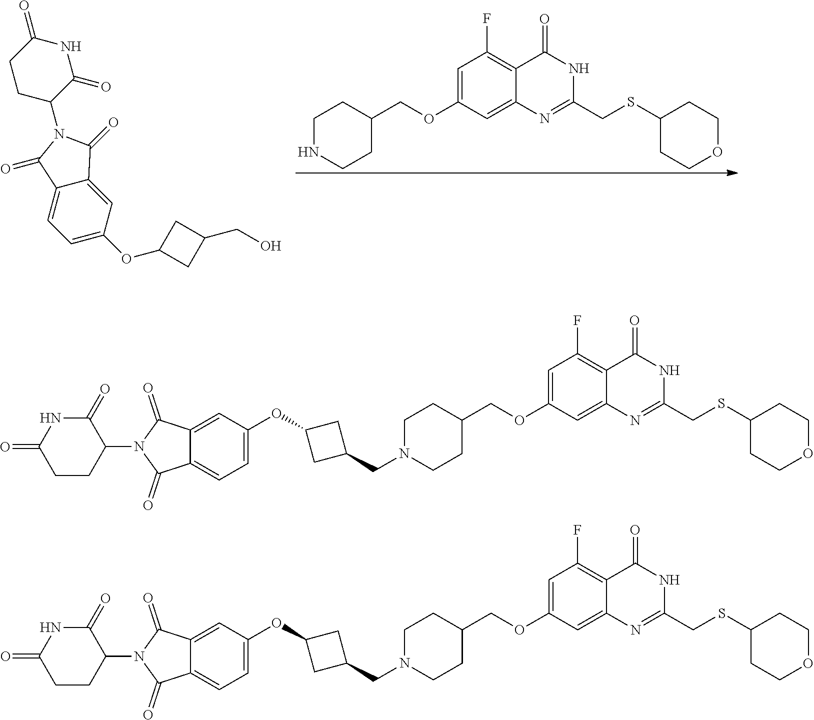 [SMILES: O=C1CCC(N2C(=O)c3ccc(OC4CC(CO)C4)cc3C2=O)C(=O)N1 ; O=c1[nH]c(CSC2CCOCC2)nc2cc(OCC3CCNCC3)cc(F)c12 ; O=C1CCC(N2C(=O)c3ccc(O[C@H]4C[C@H](CN5CCC(COc6cc(F)c7c(=O)[nH]c(CSC8CCOCC8)nc7c6)CC5)C4)cc3C2=O)C(=O)N1 ; O=C1CCC(N2C(=O)c3ccc(O[C@H]4C[C@@H](CN5CCC(COc6cc(F)c7c(=O)[nH]c(CSC8CCOCC8)nc7c6)CC5)C4)cc3C2=O)C(=O)N1]

A solution of 2-(2,6-dioxopiperidin-3-yl)-5-[3-(hydroxymethyl)cyclobutoxy]isoindole-1,3-dione (140 mg, 0.39 mmol, 1.0 equiv) and Dess-Martin reagent (182 mg, 0.43 mmol, 1.1 equiv) in DCM (4 mL) was stirred for 1 hour. To the above mixture was added 5-fluoro-2-[(oxan-4-ylsulfanyl)methyl]-7-(piperidin-4-ylmethoxy)-3H-quinazolin-4-one (159 mg, 0.39 mmol, 1.0 equiv) portion wise. The resulting mixture was stirred for 2 hours. STAB (248 mg, 1.17 mmol, 3.0 equiv) was added portion wise at 0° C. The resulting mixture was stirred for 1 hour at room temperature. The resulting mixture was concentrated under vacuum and the residue was purified by reverse phase flash chromatography with the following conditions: column, C18 silica gel; mobile phase, MeCN in water (10 mmol/L NH₄HCO₃), 0% to 47% gradient in 20 min; detector, UV 254 nm. The crude product was further purified by prep-HPLC with the following conditions (Column: X-select CSH C18 OBD Column 30*150 mm 5 μm; Mobile Phase A: water (10 mmol/L NH₄HCO₃+0.1% NH₄₀H), Mobile Phase B: ACN; Flow rate: 60 mL/min; Gradient: 35% B to 58% B in 9 min, 58% B; Wave Length: 254/220 nm; RT (min): 7.58 (Example 79a); 7.93 (Example 79b) to afford the title compounds.

Example 79a: (22 mg, 7%) as a white solid. LCMS (ESI, m/z): 748.20 [M+H]⁺; ¹H NMR (300 MHz, DMSO-d₆) δ 12.15 (s, 1H), 11.10 (s, 1H), 7.83 (d, J=8.4 Hz, 1H), 7.43-7.20 (m, 2H), 6.87 (t, J=5.5 Hz, 2H), 5.18-5.08 (m, 2H), 4.83-4.75 (m, 1H), 4.13 (d, J=6.2 Hz, 1H), 3.96 (d, J=5.7 Hz, 3H), 3.85-3.75 (m, 2H), 3.60 (s, 2H), 3.08-2.97 (m, 1H), 2.90-2.78 (m, 3H), 2.75-2.60 (m, 3H), 2.45-2.35 (m, 2H), 2.29-2.15 (m, 2H), 2.10-1.85 (m, 5H), 1.74-1.62 (m, 5H), 1.48-1.15 (m, 5H).

Example 79b: (16 mg, 5%) as a white solid. LCMS (ESI, m/z): 748.20 [M+H]⁺; ¹H NMR (300 MHz, DMSO-d₆) δ 12.15 (s, 1H), 11.10 (s, 1H), 7.81 (d, J=8.4 Hz, 1H), 7.43-7.20 (m, 2H), 6.87 (t, J=5.5 Hz, 2H), 5.15-5.01 (m, 2H), 4.13-3.75 (m, 6H), 3.60 (s, 2H), 3.08-2.97 (m, 1H), 2.61 (s, 2H), 2.90-2.76 (m, 3H), 2.30-2.10 (m, 3H), 2.08-1.82 (m, 6H), 1.81-1.55 (m, 6H), 1.54-1.18 (m, 7H).

Examples 80a and 80b were synthesized according to the procedure described for the synthesis of 2-(2,6-dioxopiperidin-3-yl)-5-((1r,3r)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione and 2-(2,6-dioxopiperidin-3-yl)-5-((1s,3s)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione (Examples 79a and 79b) using appropriate building blocks and modified reaction conditions (such as reagents, reagent ratio, temperature, and reaction time) and purification conditions as needed.

| Ex- | | |
|---|---|---|
| am- | | |
| ple | Structure | Analytical Data |
| 80a | 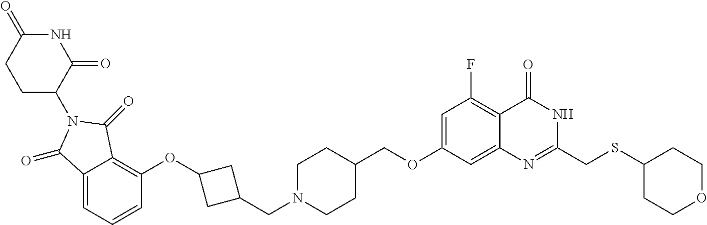 [SMILES: O=C1CCC(N2C(=O)c3cccc(OC4CC(CN5CCC(COc6cc(F)c7c(=O)[nH]c(CSC8CCOCC8)nc7c6)CC5)C4)c3C2=O)C(=O)N1] | C18 silica gel; mobile phase, MeCN in water (10 mmol/L NH₄HCO₃), 0% to 35% gradient in 20 min; detector, UV 254 nm. The crude product was purified by prep- HPLC with the following conditions: (Column: XBridge Prep OBD C18 Column, 30*150 mm, 5 μm; Mobile Phase A: water (10 mmol/L NH₄HCO₃), Mobile Phase |
| | 2-(2,6-dioxopiperidin-3-yl)-4-((1r,3r)-3-((4-(((5- | B: ACN; Flow rate: 60 |
| | fluoro-4-oxo-2-(((tetrahydro-2H-pyran-4- | mL/min; Gradient: 26% B |
| | yl)thio)methyl)-3,4-dihydroquinazolin-7- | to 45% B in 9 min, 45% |
| | yl)oxy)methyl)piperidin-1- | B; Wave Length: 254/220 |
| | yl)methyl)cyclobutoxy)isoindoline-1,3-dione | nm; RT1(min): 8.9; (53 |
| | | mg, 12%) as a white solid. |
| | | LCMS (ESI, m/z): 748.25 |
| | | [M + H]⁺, ¹H NMR (300 |
| | | MHz, DMSO-d₆) δ 12.15 |
| | | (s, 1H), 11.10 (s, 1H), |
| | | 7.72-6.98 (m, 1H), 7.43 |
| | | (d, J = 7.2 Hz, 1H), 7.33 |
| | | (d, J = 8.5 Hz, 1H), 6.86 |
| | | (t, J = 5.4 Hz, 2H), 5.12- |
| | | 5.02 (m, 1H), 4.85-4.74 |
| | | (m, 1H), 3.96 (d, J = 5.8 |
| | | Hz, 2H), 3.80-3.71 (m, |
| | | 2H), 3.60 (s, 2H), 3.10- |
| | | 2.97 (m, 2H), 2.90-2.72 |
| | | (m, 3H), 2.665-2.50 (m, |
| | | 3H), 2.40-2.35 (m, 3H), |
| | | 2.21-2.10 (m, 2H), 1.98- |
| | | 1.82 (m, 4H), 1.80-1.65 |
| | | (m, 5H), 1.52-1.19 (m, |
| | | 5H). |
| 80b | 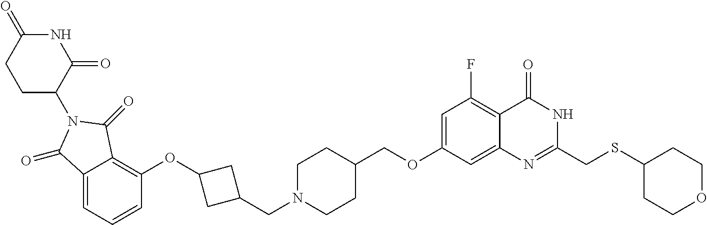 [SMILES: O=C1CCC(N2C(=O)c3cccc(OC4CC(CN5CCC(COc6cc(F)c7c(=O)[nH]c(CSC8CCOCC8)nc7c6)CC5)C4)c3C2=O)C(=O)N1] | (Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 26% B to 45% B in 9 min, 45% B; Wave Length: 254/220 nm; RT2(min): 9.2; (12 mg, 5%) as a white solid. LCMS (ESI, m/z): 748.20 [M + H]⁺, ¹H NMR (300 |
| | 2-(2,6-dioxopiperidin-3-yl)-4-((1s,3s)-3-((4-(((5- | MHz, DMSO-d₆) δ 7.80 |
| | fluoro-4-oxo-2-(((tetrahydro-2H-pyran-4- | (t, J = 7.9 Hz, 1H), 7.46 |
| | yl)thio)methyl)-3,4-dihydroquinazolin-7- | (d, J = 7.3 Hz, 1H), 7.22 |
| | yl)oxy)methyl)piperidin-1- | (d, J = 8.5 Hz, 1H), 6.91- |
| | yl)methyl)cyclobutoxy)isoindoline-1,3-dione | 6.80 (m, 2H), 5.11-5.01 |
| | | (m, 2H), 4.00 (d, J = 5.8 |
| | | Hz, 2H), 3.91-3.74 (m, |
| | | 4H), 3.47 (d, J = 11.0 Hz, |
| | | 2H), 3.37-3.22 (m, 4H), |
| | | 3.09-2.77 (m, 5H), 2.65- |
| | | 2.55 (m, 1H), 2.57-2.41 |
| | | (m, 4H), 2.10-1.80 (m, |
| | | 7H), 1.63-1.33 (m, 4H). |

### Example 81: Synthesis of 3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutoxy)phenyl) amino)piperidine-2,6-dione

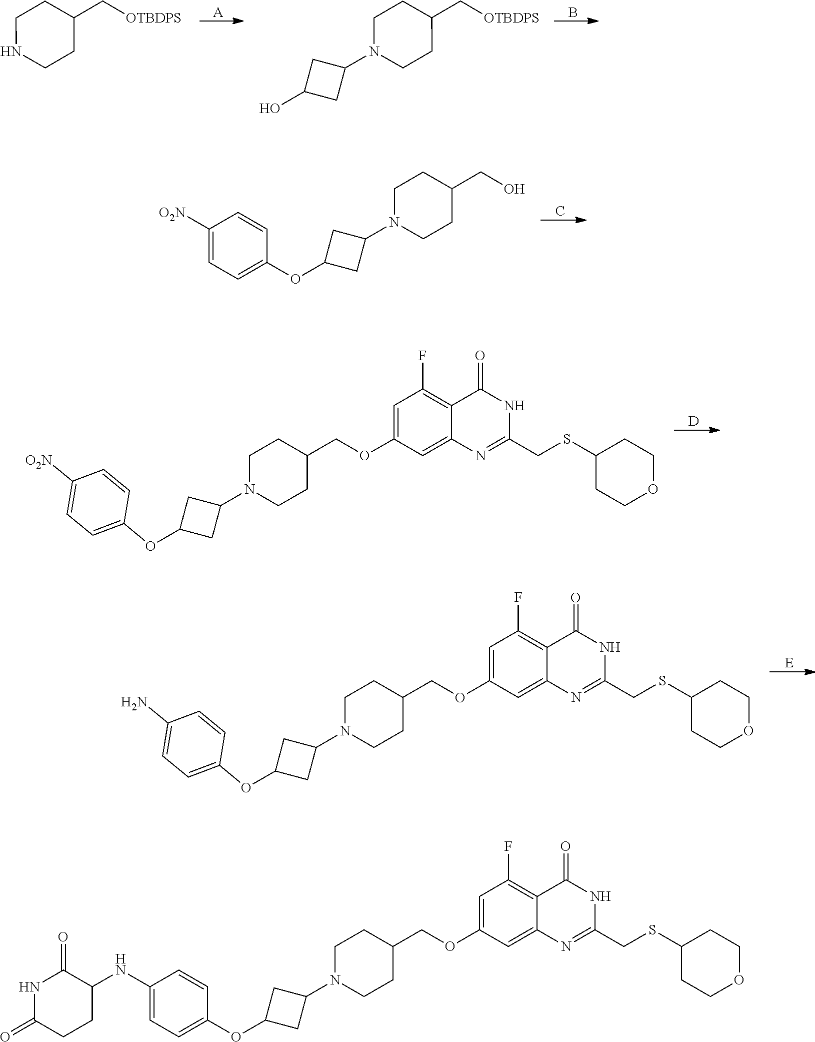

### Step A

A solution of 4-(((tert-butyldiphenylsilyl)oxy)methyl)piperidine (6.8 g, 19.2 mmol, 1.0 equiv), 3-hydroxycyclobutan-1-one (1.66 g, 19.2 mmol, 1.0 equiv) and STAB (20.4 g, 96.2 mmol, 5.0 equiv) in DCE (70 mL) was stirred for 1 hour. The resulting mixture was concentrated under vacuum. The residue was purified by reverse phase flash chromatography with the following conditions: column, C18 silica gel; mobile phase, MeOH in water (10 mmol/L NH₄HCO₃), 0% to 93% gradient in 40 min; detector, UV 254 nm to afford of 3-(4-(((tert-butyldiphenylsilyl)oxy)methyl)piperidin-1-yl)cyclobutan-1-ol (6.32 g, 78%) as a yellow oil. LCMS (ESI, m/z): 424.25 [M+H]⁺.

### Step B

A solution of 3-(4-(((tert-butyldiphenylsilyl)oxy)methyl)piperidin-1-yl)cyclobutan-1-ol (6.32 g, 14.9 mmol, 1.0 equiv), 1-fluoro-4-nitrobenzene (2.11 g, 14.9 mmol, 1.0 equiv) and t-BuOK (3.35 g, 29.9 mmol, 2.0 equiv) in DMSO (100 mL) was stirred overnight at 80° C. The resulting mixture was diluted with water (200 mL) and extracted with EtOAc (7×100 mL). The combined organic layers were washed with brine (3×100 mL), dried over anhydrous Na₂SO₄ and filtered. The filtrate was concentrated under reduced pressure and the crude product was purified by normal phase chromatography eluting with CH₂Cl₂/MeOH (5:1) to afford (1-(3-(4-nitrophenoxy)cyclobutyl)piperidin-4-yl)methanol (1.75 g, 38%) as a yellow oil. LCMS (ESI, m/z): 307.15 [M+H]⁺.

### Step C

A solution of (1-(3-(4-nitrophenoxy)cyclobutyl)piperidin-4-yl)methanol (1.5 g, 4.90 mmol, 1.0 equiv), 7-bromo-5-fluoro-2-(((tetrahydro-2H-pyran-4-yl)thio)methyl)quinazolin-4(3H)-one (1.83 g, 4.90 mmol, 1.0 equiv), Cs₂CO₃ (3.19 g, 9.79 mmol, 2.0 equiv), Pd₂(allyl)₂Cl₂ (17.9 mg, 0.049 mmol, 0.01 equiv) and RockPhos (0.23 g, 0.490 mmol, 0.10 equiv) in toluene (10 mL) was stirred overnight at 100° C. The resulting mixture was concentrated under vacuum. The residue was purified by reverse flash chromatography with the following conditions: column, C18 silica gel; mobile phase, MeCN in water (10 mmol/L NH₄HCO₃), 0% to 60% gradient in 30 min; detector, UV 254 nm to afford 5-fluoro-7-((1-(3-(4-nitrophenoxy)cyclobutyl)piperidin-4-yl)methoxy)-2-(((tetrahydro-2H-pyran-4-yl)thio)methyl)quinazolin-4(3H)-one (450 mg, 15%) as a brown oil. LCMS (ESI, m/z): 599.25 [M+H]⁺.

### Step D

A solution of 5-fluoro-7-((1-(3-(4-nitrophenoxy)cyclobutyl)piperidin-4-yl)methoxy)-2-(((tetrahydro-2H-pyran-4-yl)thio)methyl)quinazolin-4(3H)-one (440 mg, 0.74 mmol, 1.0 equiv), Fe (121 mg, 2.17 mmol, 10 equiv), and NH₄Cl (157 mg, 2.94 mmol, 4.0 equiv) in water (2 mL) and EtOH (8 mL) was stirred overnight at 80° C. The resulting mixture was concentrated under vacuum. The residue was purified by reverse flash chromatography with the following conditions: column, C18 silica gel; mobile phase, MeCN in water (10 mmol/L NH₄HCO₃), 0% to 50% gradient in 30 min; detector, UV 254 nm to afford 7-((1-(3-(4-aminophenoxy)cyclobutyl)piperidin-4-yl)methoxy)-5-fluoro-2-(((tetrahydro-2H-pyran-4-yl)thio)methyl)quinazolin-4(3H)-one (152 mg, 36%) as a brown oil. LCMS (ESI, m/z): 569.30 [M+H]⁺.

### Step E

A solution of 7-((1-(3-(4-aminophenoxy)cyclobutyl)piperidin-4-yl)methoxy)-5-fluoro-2-(((tetrahydro-2H-pyran-4-yl)thio)methyl)quinazolin-4(3H)-one (130 mg, 0.23 mmol, 1.0 equiv), 3-bromopiperidine-2,6-dione (43.9 mg, 0.23 mmol, 1.0 equiv) and NaHCO₃ (38.4 mg, 0.46 mmol, 2.0 equiv) in ACN (10 mL) was stirred overnight at 90° C. The resulting mixture was concentrated under vacuum. After concentration, the residue was purified by C18 reverse phase chromatography eluting with water/ACN (28:72) and further purified by prep-HPLC with the following condition ((Column: XSelect CSH Prep C18 OBD Column, 19*250 mm, 5 μm; Mobile Phase A: water (10 mmol/L NH₄HCO₃), Mobile Phase B: ACN; Flow rate: 20 mL/min; Gradient: 40% B to 45% B in 8 min, 45% B; Wave Length: 220 nm; RT1 (min): 8) to afford 3-((4-(3-(4-(((5-fluoro-4-oxo-2-(((tetrahydro-2H-pyran-4-yl)thio)methyl)-3,4-dihydroquinazolin-7-yl)oxy)methyl) piperidin-1-yl)cyclobutoxy)phenyl)amino)piperidine-2,6-dione (33.8 mg, 22%) as a white solid. LCMS (ESI, m/z): 680.35 [M+H]⁺. ¹H NMR (300 MHz, DMSO-d₆) δ 12.14 (s, 1H), 10.74 (s, 1H), 6.96-6.85 (m, 2H), 6.65-6.53 (m, 5H), 5.42 (d, J=7.1 Hz, 1H), 4.65-4.59 (m, 1H), 4.21-4.17 (m, 1H), 3.99 (d, J=5.6 Hz, 2H), 3.86-3.79 (m, 2H), 3.62 (s, 2H), 3.40-3.29 (m, 3H), 3.13-3.05 (m, 2H), 3.03-2.98 (m, 2H), 2.90 (s, 1H), 2.61-2.70 (m, 1H), 2.40-2.20 (m, 2H), 2.05-2.17 (m, 3H), 1.91-1.65 (m, 10H), 1.62-1.18 (m, 7H).

### Example 82: Synthesis of 3-((4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione

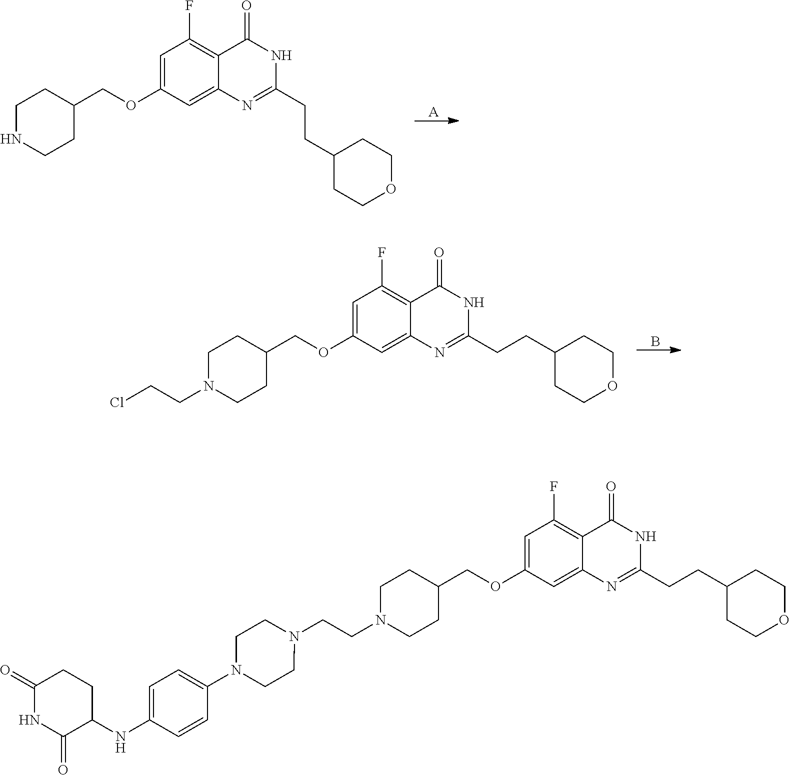

### Step A

A mixture of 5-fluoro-7-(piperidin-4-ylmethoxy)-2-(2-(tetrahydro-2H-pyran-4-yl)ethyl)quinazolin-4(3H)-one (200 mg, 0.51 mmol, 1.0 equiv), chloroacetaldehyde (202 mg, 2.57 mmol, 5.0 equiv) and STAB (218 mg, 1.03 mmol, 2.0 equiv) in DCM (3 mL) was stirred for 2 hours. The residue was purified by silica gel column chromatography, eluting with PE/EtOAc (1:1) to 7-((1-(2-chloroethyl)piperidin-4-yl)methoxy)-5-fluoro-2-(2-(tetrahydro-2H-pyran-4-yl)ethyl)quinazolin-4(3H)-one (100 mg, 40%) as a white solid. LCMS (ESI, m/z): 452.20 [M+H]⁺.

### Step B

A mixture of 7-((1-(2-chloroethyl)piperidin-4-yl)methoxy)-5-fluoro-2-(2-(tetrahydro-2H-pyran-4-yl)ethyl)quinazolin-4(3H)-one (100 mg, 0.22 mmol, 1.0 equiv), 3-((4-(piperazin-1-yl)phenyl)amino)piperidine-2,6-dione (95.7 mg, 0.33 mmol, 1.5 equiv) and DIEA (143 mg, 1.11 mmol, 5.0 equiv) in DMSO (2 mL) was stirred for 2 hours at 80° C. The crude product was purified directly by reverse phase flash with the following conditions (ACN:water=30:70) to afford the desired product which was further purified by prep-HPLC with the following conditions: Column: Xselect CSH F-Phenyl OBD column, 19*250 mm, 5 μm; Mobile Phase A: water (0.1% FA), Mobile Phase B: ACN; Flow rate: 20 mL/min; Gradient: 7% B to 11% B in 8 min, 11% B; Wave Length: 220 nm; RT1 (min): 12 to afford 3-((4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione (15 mg, 10%) as a dark solid. LCMS (ESI, m/z): 704.35 [M+H]⁺. ¹H NMR (300 MHz, DMSO-d₆) δ 12.03 (s, 1H), 10.73 (s, 1H), 6.88-6.76 (m, 4H), 6.61 (d, J=8.9 Hz, 2H), 5.35 (br, 1H), 4.23-4.12 (m, 1H), 3.99 (d, J=5.9 Hz, 2H), 3.83 (dd, J=11.7, 3.1 Hz, 2H), 3.32-3.21 (m, 4H), 3.18-3.01 (m, 2H), 2.98-2.91 (m, 3H), 2.82-2.68 (m, 2H), 2.70-2.56 (m, 10H), 2.35-2.25 (m, 2H), 2.17-2.05 (m, 1H), 1.86-1.76 (m, 3H), 1.71-1.55 (m, 4H), 1.53-1.36 (m, 2H), 1.27-1.08 (m, 3H).

Example 83 was synthesized according to the procedure described for the synthesis 3-((4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione (Example 82) using appropriate building blocks and modified reaction conditions (such as reagents, reagent ratio, temperature, and reaction time) and purification conditions as needed.

| Ex- | | Ana- |
|---|---|---|
| am- | | lytical |
| ple | Structure | Data |
| 83 | 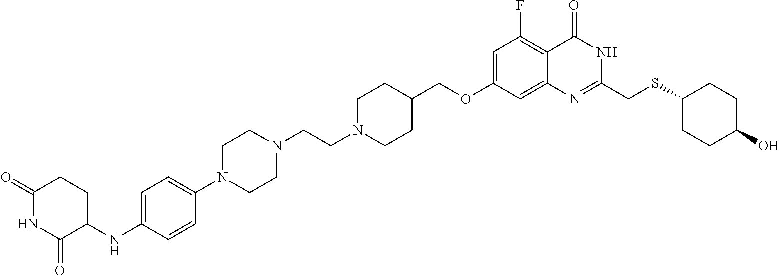 [SMILES: O=C1CCC(Nc2ccc(N3CCN(CCN4CCC(COc5cc(F)c6c(=O)[nH]c(CS[C@H]7CC[C@H](O)CC7)nc6c5)CC4)CC3)cc2)C(=O)N1] | LCMS (ESI, m/z): 736.50 [M + H]⁺; 1H NMR (400 MHz, DMSO-d6) δ 12.18 (s, 1H), 10.71 (s, 1H), 7.25-7.15 (m, 2H), 6.95-6.82 (m, 2H), 6.75 (d, J = 8.4 Hz, 1H), 6.61 (d, J = 8.4 |
| | 3-((4-(4-(2-(4-(((5-fluoro-2-((((1r,4r)-4- | Hz, 1H), |
| | hydroxycyclohexyl)thio)methyl)-4-oxo-3,4- | 5.36 (d, J = |
| | dihydroquinazolin-7-yl)oxy)methyl)piperidin-1- | 7.4 Hz, 1H), |
| | yl)ethyl)piperazin-1-yl)phenyl)amino)piperidine-2,6- | 4.55-4.45 |
| | dione | (m, 1H), |
| | | 4.23-4.15 |
| | | (m, 1H), |
| | | 3.98 (d, |
| | | J = 5.8 Hz, |
| | | 1H), 3.81 |
| | | (d, J = |
| | | 7.2 Hz, |
| | | 1H), 3.58 |
| | | (s, 1H), |
| | | 3.54-3.40 |
| | | (m, 4H), |
| | | 3.28-3.15 |
| | | (m, 4H), |
| | | 3.00-2.86 |
| | | (m, 3H), |
| | | 2.78-2.67 |
| | | (m, 1H), |
| | | 2.60-2.50 |
| | | (m, 1H), |
| | | 2.46-2.41 |
| | | (m, 2H), |
| | | 2.17-1.92 |
| | | (m, 3H), |
| | | 1.90-1.65 |
| | | (m, 4H), |
| | | 1.61-1.43 |
| | | (m, 2H), |
| | | 1.40-1.00 |
| | | (m, 10H) |

### Example 84: Synthesis of 3-((4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)phenyl)amino)piperidine-2,6-dione

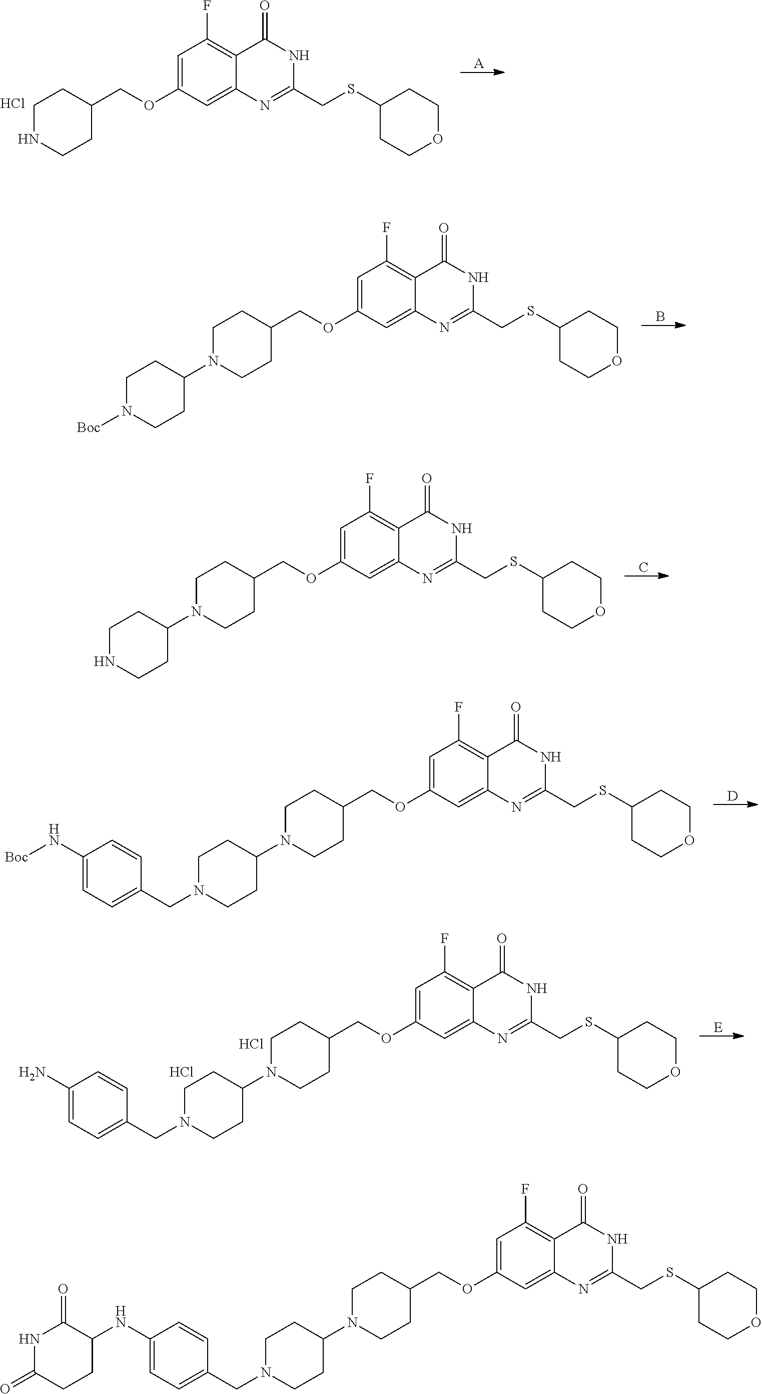

### Step A

To a solution of 5-fluoro-7-(piperidin-4-ylmethoxy)-2-(((tetrahydro-2H-pyran-4-yl)thio)methyl)quinazolin-4(3H)-one (5.0 g, 12.3 mmol, 1.0 equiv) in DCE (30 mL) was added tert-butyl 4-oxopiperidine-1-carboxylate (4.89 g, 24.5 mmol, 2.0 equiv) and the reaction was stirred overnight. STAB (13.0 g, 61.4 mmol, 5.0 equiv) was added in portions. The mixture was stirred for 4 hours at room temperature. The resulting mixture was concentrated under reduced pressure. The residue was purified by reverse phase flash chromatography with the following conditions: column, C18 silica gel; mobile phase, MeCN in water (10 mmol/L NH₄HCO₃), 10% to 60% gradient in 30 min; detector, UV 254 nm. This afforded tert-butyl 4-(((5-fluoro-4-oxo-2-(((tetrahydro-2H-pyran-4-yl)thio)methyl)-3,4-dihydroquinazolin-7-yl)oxy)methyl)-[1,4′-bipiperidine]-1′-carboxylate (1.1 g, 15%) as a white solid. LCMS (ESI, m/z): 591.25 [M+H]⁺.

### Step B

A solution of tert-butyl 4-(((5-fluoro-4-oxo-2-(((tetrahydro-2H-pyran-4-yl)thio)methyl)-3,4-dihydroquinazolin-7-yl)oxy)methyl)-[1,4′-bipiperidine]-1′-carboxylate (1.1 g, 1.86 mmol, 1.0 equiv) in HCl in 1,4-dioxane (25 mL, 4M) was stirred for 30 mins. The reaction was monitored by LCMS. After completion, the resulting mixture was concentrated under reduced pressure to afford 7-([1,4′-bipiperidin]-4-ylmethoxy)-5-fluoro-2-(((tetrahydro-2H-pyran-4-yl)thio)methyl)quinazolin-4(3H)-one (1.7 g) as a white solid. The crude material was used in the next step directly without further purification. LCMS (ESI, m/z): 491.20[M+H]⁺.

### Step C

A solution of 7-([1,4′-bipiperidin]-4-ylmethoxy)-5-fluoro-2-(((tetrahydro-2H-pyran-4-yl)thio)methyl)quinazolin-4(3H)-one (1.2 g, 2.45 mmol, 1.0 equiv) in DCE (20 mL) was treated with tert-butyl N-(4-formylphenyl)carbamate (1.08 g, 4.89 mmol, 2.0 equiv) for 2 hours then STAB (2.59 g, 12.2 mmol, 5.0 equiv) was added in portions. The resulting mixture was stirred overnight. The resulting mixture was concentrated under reduced pressure. The residue was purified by reverse phase flash chromatography with the following conditions: column, C18 silica gel; mobile phase, MeCN in water (10 mmol/L NH₄HCO₃), 10% to 60% gradient in 30 min; detector, UV 254 nm. This afforded tert-butyl (4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)phenyl)carbamate (1 g, 59%) as a white solid. LCMS (ESI, m/z): 696.30 [M+H]⁺.

### Step D

A solution of tert-butyl (4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)phenyl)carbamate (500 mg, 0.72 mmol, 1.0 equiv) in HCl in 1,4-dioxane (10 mL, 4M) was stirred for 30 mins. The resulting mixture was concentrated under reduced pressure. The crude product was basified to pH 8 with saturated NaHCO₃ (aq.) and then concentrated. The residue was purified by reverse phase flash chromatography with the following conditions: column, C18 silica gel; mobile phase, MeCN in water (10 mmol/L NH₄HCO₃), 10% to 60% gradient in 20 min; detector, UV 254 nm to afford 7-((1′-(4-aminobenzyl)-[1,4′-bipiperidin]-4-yl)methoxy)-5-fluoro-2-(((tetrahydro-2H-pyran-4-yl)thio)methyl)quinazolin-4(3H)-one (260 mg, 57%) as a white solid. LCMS (ESI, m/z): 596.35 [M+H]⁺.

### Step E

A solution of 7-((1′-(4-aminobenzyl)-[1,4′-bipiperidin]-4-yl)methoxy)-5-fluoro-2-(((tetrahydro-2H-pyran-4-yl)thio)methyl)quinazolin-4(3H)-one (250 mg, 0.42 mmol, 1.0 equiv), 3-bromopiperidine-2,6-dione (80.6 mg, 0.42 mmol, 1.0 equiv) and NaHCO₃ (70.5 mg, 0.84 mmol, 2.0 equiv) in ACN (10 mL) was stirred for two days at 90° C. The resulting mixture was concentrated under reduced pressure. The residue was purified by C18 reverse phase chromatography eluting with water/ACN (59:41) and further purified by prep-HPLC with the following condition Column: XBridge Prep Phenyl OBD Column, 19*250 mm, 5 μm; Mobile Phase A: water (10 mmol/L NH₄HCO₃), Mobile Phase B: ACN; Flow rate: 25 mL/min; Gradient: 30% B to 40% B in 10 min, 40% B; Wave Length: 254 nm; RT1 (min): 9 to afford 3-((4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)phenyl)amino)piperidine-2,6-dione (90 mg, 30%) as a white solid. LCMS (ESI, m/z): 707.30 [M+H]⁺; ¹H NMR (300 MHz, DMSO-d₆) δ 12.12 (s, 1H), 10.76 (s, 1H), 6.98 (d, J=8.2 Hz, 2H), 6.92-6.81 (m, 2H), 6.62 (d, J=8.3 Hz, 2H), 5.72 (d, J=7.4 Hz, 1H), 4.35-4.21 (m, 1H), 3.95 (d, J=5.6 Hz, 2H), 3.87-3.75 (m, 2H), 3.62 (s, 2H), 3.40-3.36 (m, 3H), 3.35-3.28 (m, 2H), 3.14-2.97 (m, 1H), 2.90-2.81 (m, 4H), 2.77-2.66 (m, 1H), 2.65-2.52 (m, 1H), 2.21-2.06 (m, 4H), 1.97-1.60 (m, 10H), 1.54-1.20 (m, 5H).

### Example 85: Synthesis of 2-(2,6-dioxopiperidin-3-yl)-5-(4-(4-((5-fluoro-4-oxo-2-(((tetrahydro-2H-pyran-4-yl)thio)methyl)-3,4-dihydroquinazolin-7-yl)methyl)piperazin-1-yl)piperidin-1-yl)isoindoline-1,3-dione

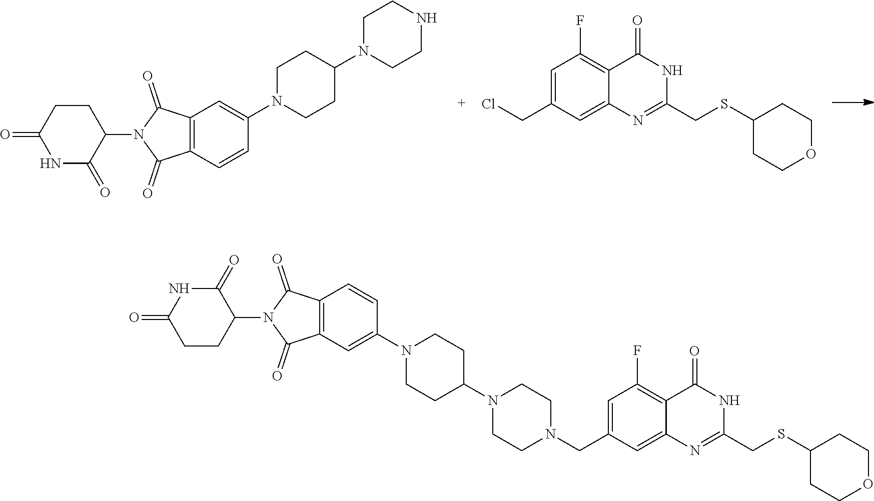 [SMILES: O=C1CCC(N2C(=O)c3ccc(N4CCC(N5CCNCC5)CC4)cc3C2=O)C(=O)N1 ; O=c1[nH]c(CSC2CCOCC2)nc2cc(CCl)cc(F)c12 ; O=C1CCC(N2C(=O)c3ccc(N4CCC(N5CCN(Cc6cc(F)c7c(=O)[nH]c(CSC8CCOCC8)nc7c6)CC5)CC4)cc3C2=O)C(=O)N1]

A solution of 7-(chloromethyl)-5-fluoro-2-(((tetrahydro-2H-pyran-4-yl)thio)methyl)quinazolin-4(3H)-one (30 mg, 0.088 mmol, 1.0 equiv), 2-(2,6-dioxopiperidin-3-yl)-5-[4-(piperazin-1-yl)piperidin-1-yl]isoindole-1,3-dione (37 mg, 0.088 mmol, 1.0 equiv) and DIEA (33.9 mg, 0.26 mmol, 3.0 equiv) in ACN (2 mL) was stirred for 1 day at 60° C. under nitrogen atmosphere. The residue was purified by reverse phase flash chromatography with the following conditions: column, C18 silica gel; mobile phase, MeCN in water (10 mmol/L NH₄HCO₃), 0% to 36% gradient in 20 min; detector, UV 254 nm. The crude product was further purified by prep-HPLC with the following conditions (Column: Xselect CSH C18 OBD Column 30*150 mm 5 μm, n; Mobile Phase A: water (0.05% FA), Mobile Phase B: ACN; Flow rate: 60 mL/min; Gradient: 2% B to 28% B in 9 min, 28% B; Wave Length: 254/220 nm; RT1 (min): 8.17) to afford 2-(2,6-dioxopiperidin-3-yl)-5-(4-(4-((5-fluoro-4-oxo-2-(((tetrahydro-2H-pyran-4-yl)thio)methyl)-3,4-dihydroquinazolin-7-yl)methyl)piperazin-1-yl)piperidin-1-yl)isoindoline-1,3-dione (22.7 mg, 35%) as a yellow solid. LCMS(ES, m/z): 732.25[M+H]⁺, ¹H NMR (300 MHz, DMSO-d₆) δ 12.29 (s, 1H), 11.07 (s, 1H), 7.64 (d, J=8.5 Hz, 1H), 7.36-7.27 (m, 2H), 7.23 (d, J=9.0 Hz, 1H), 7.19-7.09 (m, 1H), 5.11-5.04 (m, 1H), 4.05 (d, J=12.8 Hz, 2H), 3.85-3.75 (m, 2H), 3.62 (s, 2H), 3.55 (s, 2H), 3.30-3.18 (m, 6H), 3.15-2.75 (m, 5H), 2.65-2.58 (m, 1H), 2.45-2.32 (m, 5H), 2.10-1.80 (m, 5H), 1.52-1.33 (m, 4H).

### Example 86: Synthesis of 3-((4-(4-(4-(2-(5-fluoro-4-oxo-2-(((tetrahydro-2H-pyran-4-yl)thio)methyl)-3,4-dihydroquinazolin-7-yl)ethyl)piperazin-1-yl)piperidin-1-yl)phenyl)amino)piperidine-2,6-dione

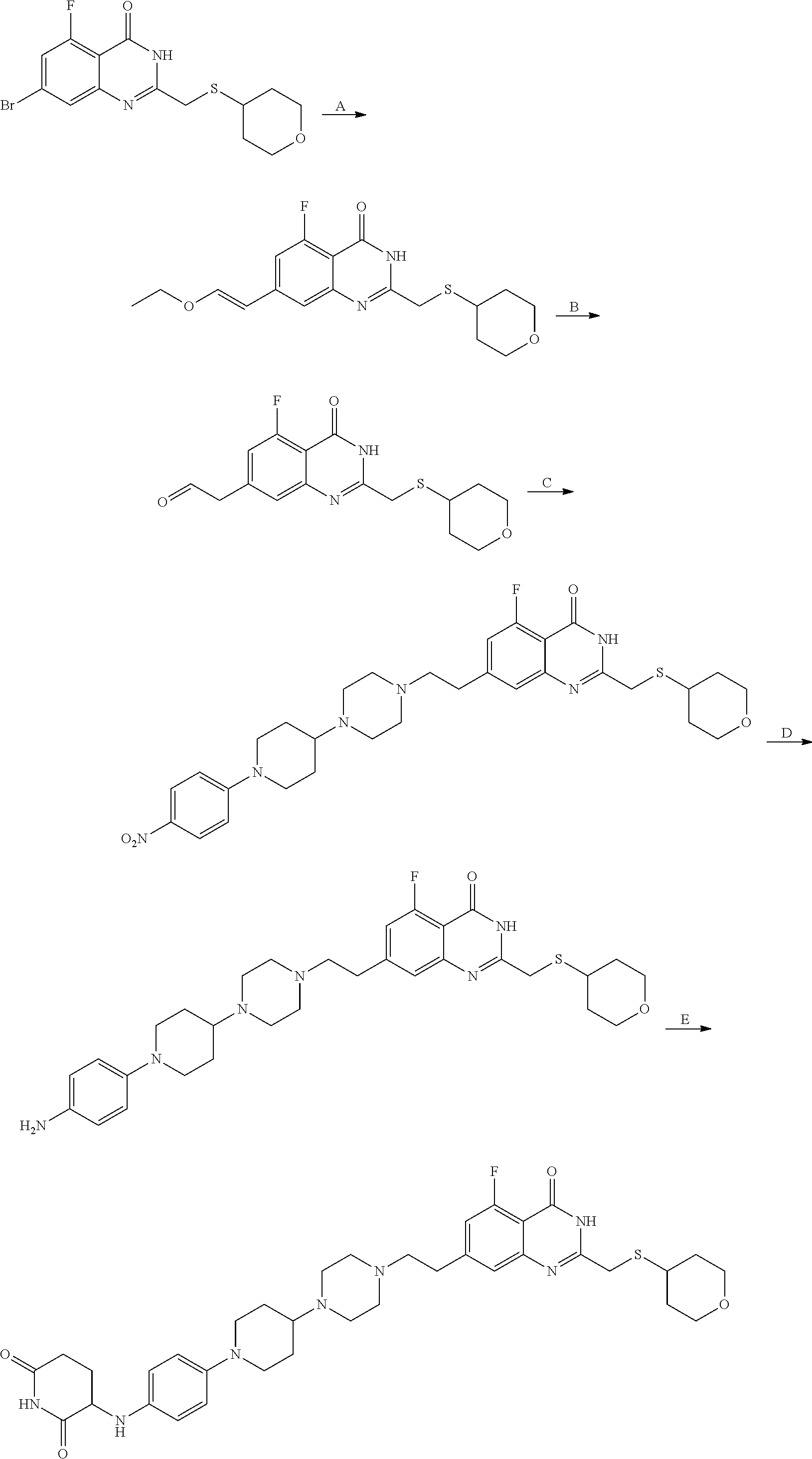

### Step A

A solution of 7-bromo-5-fluoro-2-(((tetrahydro-2H-pyran-4-yl)thio)methyl)quinazolin-4(3H)-one (2.30 g, 6.16 mmol, 1.0 equiv), (E)-2-(2-ethoxyvinyl)-4,4,5,5-tetramethyl-1,3,2-dioxaborolane (1.22 g, 6.16 mmol, 1.0 equiv), Pd(dppf)Cl₂ (451 mg, 0.62 mmol, 0.1 equiv) and K₂CO₃ (1.70 g, 12.3 mmol, 2.0 equiv) in dioxane (20 ml) and water (5 mL) was stirred for 2 hours at 70° C. After concentration, the residue was purified by silica gel column chromatography, eluting with PE/EtOAc (1:1) to afford (E)-7-(2-ethoxyvinyl)-5-fluoro-2-(((tetrahydro-2H-pyran-4-yl)thio)methyl)quinazolin-4(3H)-one (1.6 g, 71%) as a yellow oil. LCMS (ESI, m/z): 365.10 [M+H]⁺.

### Step B

A solution of (E)-7-(2-ethoxyvinyl)-5-fluoro-2-(((tetrahydro-2H-pyran-4-yl)thio)methyl)quinazolin-4(3H)-one (1.4 g, 3.84 mmol, 1.0 equiv) in TFA (5 mL) and DCM (25 mL) was stirred for 1 hour. The resulting solution was concentrated under vacuum. The residue was basified to pH 7 with saturated aqueous NaHCO₃ and extracted with CH₂Cl₂ (3×10 mL). The combined organic layers were concentrated under vacuum to afford 2-(5-fluoro-4-oxo-2-(((tetrahydro-2H-pyran-4-yl)thio)methyl)-3,4-dihydroquinazolin-7-yl)acetaldehyde (600 mg, 46%) as a brown yellow solid that was used without further purification. LCMS (ESI, m/z): 337.10 [M+H]⁺.

### Step C

A solution of 2-(5-fluoro-4-oxo-2-(((tetrahydro-2H-pyran-4-yl)thio)methyl)-3,4-dihydroquinazolin-7-yl)acetaldehyde (600 mg, 1.78 mmol, 1.0 equiv), 1-(1-(4-nitrophenyl)piperidin-4-yl)piperazine (518 mg, 1.78 mmol, 1.0 equiv) and STAB (756 mg, 3.57 mmol, 2.0 equiv) in DCE (10 mL) was stirred for 2 hours. The residue was purified by silica gel column chromatography, eluting with CH₂Cl₂/MeOH (5:1). The resulting mixture was concentrated under reduced pressure to afford 5-fluoro-7-(2-(4-(1-(4-nitrophenyl)piperidin-4-yl)piperazin-1-yl)ethyl)-2-(((tetrahydro-2H-pyran-4-yl)thio)methyl)quinazolin-4(3H)-one (176 mg, 16%) as a yellow solid. LCMS (ESI, m/z): 611.10 [M+H]⁺.

### Step D

A solution of 5-fluoro-7-(2-(4-(1-(4-nitrophenyl)piperidin-4-yl)piperazin-1-yl)ethyl)-2-(((tetrahydro-2H-pyran-4-yl)thio)methyl)quinazolin-4(3H)-one (176 mg, 0.29 mmol, 1.0 equiv), NH₄Cl (31 mg, 0.58 mmol, 2.0 equiv) and Fe (81 mg, 1.44 mmol, 5.0 equiv) in EtOH (2.5 mL) and water (0.5 mL) was stirred for 2 hours at 80° C. The resulting mixture was filtered and the filter cake was washed with EtOH (3×10 mL). The filtrate was concentrated under reduced pressure. The residue was purified by silica gel column chromatography, eluting with PE/EtOAc (1:1) to afford 7-(2-(4-(1-(4-aminophenyl)piperidin-4-yl)piperazin-1-yl)ethyl)-5-fluoro-2-(((tetrahydro-2H-pyran-4-yl)thio)methyl)quinazolin-4(3H)-one (120 mg, 72%) as a yellow solid. LCMS (ESI, m/z): 581.10 [M+H]⁺.

### Step E

A solution of 7-(2-(4-(1-(4-aminophenyl)piperidin-4-yl)piperazin-1-yl)ethyl)-5-fluoro-2-(((tetrahydro-2H-pyran-4-yl)thio)methyl)quinazolin-4(3H)-one (120 mg, 0.21 mmol, 1.0 equiv), 3-bromopiperidine-2,6-dione (40 mg, 0.21 mmol, 1.0 equiv) and NaHCO₃ (52 mg, 0.62 mmol, 3.0 equiv) in ACN (5 mL) was stirred for 1 hour at 90° C. The residue was purified by silica gel column chromatography, eluting with PE/EtOAc (1:1). The resulting product was further purified by prep-HPLC with the following conditions: Column: X-Bridge Prep Phenyl OBD Column, 19*250 mm, 5 μm; Mobile Phase A: water (10 mmol/L NH₄HCO₃), Mobile Phase B: ACN; Flow rate: 25 mL/min; Gradient: 40% B to 45% B in 11 min, 45% B; Wave Length: 254 nm; RT1 (min): 10 to afford 3-((4-(4-(4-(2-(5-fluoro-4-oxo-2-(((tetrahydro-2H-pyran-4-yl)thio)methyl)-3,4-dihydroquinazolin-7-yl)ethyl)piperazin-1-yl)piperidin-1-yl)phenyl)amino)piperidine-2,6-dione (3.4 mg, 2%) as a white solid. LCMS (ESI, m/z): 692.35 [M+H]⁺. ¹H NMR (400 MHz, DMSO-d₆) δ 12.24 (s, 1H), 10.76 (s, 1H), 7.30 (s, 1H), 7.16 (d, J=8.0 Hz, 1H), 6.65 (d, J=8.8 Hz, 2H), 6.60 (d, J=12.0 Hz, 2H), 5.37 (d, J=7.3 Hz, 1H), 4.24-4.14 (m, 1H), 3.87-3.81 (m, 2H), 3.63 (s, 2H), 3.42 (d, J=11.5 Hz, 2H), 3.35-3.30 (m, 2H), 3.12-3.01 (m, 1H), 2.90-2.81 (m, 2H), 2.79-2.69 (m, 1H), 2.62-2.53 (m, 4H), 2.49-2.38 (m, 9H), 2.24-2.16 (m, 1H), 2.15-2.06 (m, 1H), 1.90-1.78 (m, 5H), 1.54-1.41 (m, 4H).

### Example 87: Synthesis of 3-((4-((1-(4-(((5-fluoro-4-oxo-2-(((tetrahydro-2H-pyran-4-yl)thio)methyl)-3,4-dihydroquinazolin-7-yl)oxy)methyl)cyclohexyl)piperidin-4-yl)oxy)phenyl)amino)piperidine-2,6-dione

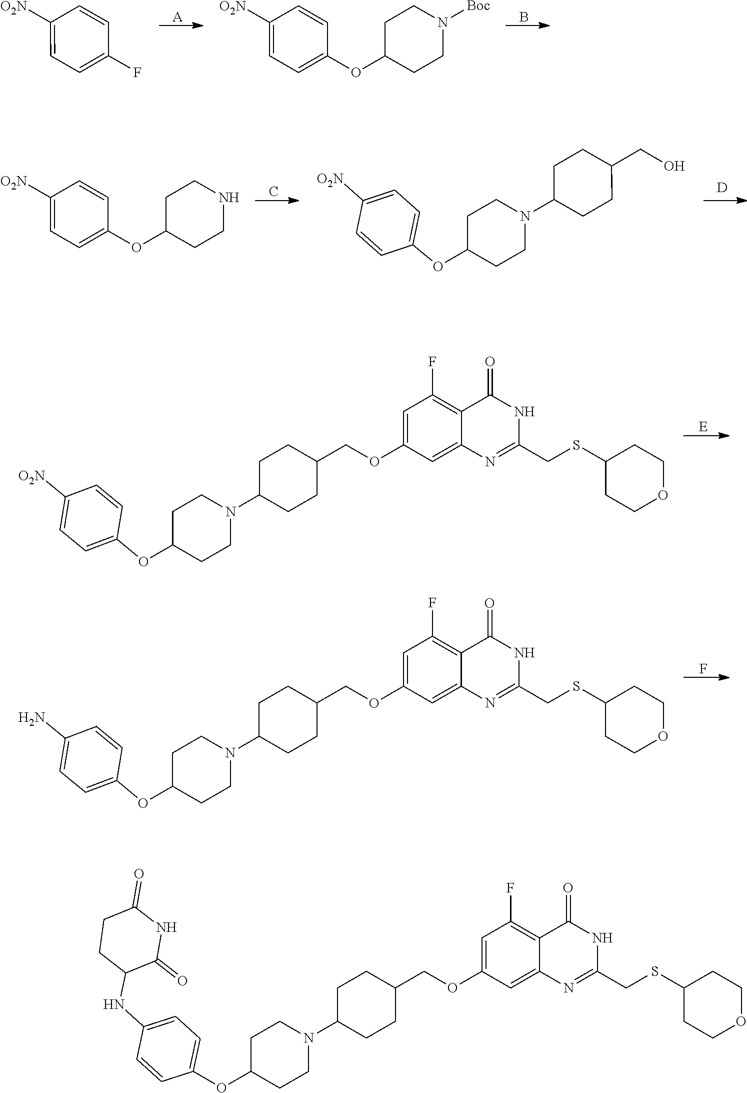

### Step A

A solution of 1-fluoro-4-nitrobenzene (5.00 g, 35.4 mmol, 1.0 equiv), tert-butyl 4-hydroxypiperidine-1-carboxylate (7.13 g, 35.4 mmol, 1.0 equiv), and t-BuOK (11.9 g, 106 mmol, 3.0 equiv) in THF (30 mL) was stirred for 2 hours. The resulting mixture was concentrated under reduced pressure. The residue was purified by silica gel column chromatography, eluting with PE/EtOAc (21:29) to afford tert-butyl 4-(4-nitrophenoxy)piperidine-1-carboxylate (5.75 g, 50%) as a yellow solid. LCMS (ESI, m/z): 323.15 [M+H]⁺.

### Step B

A solution of tert-butyl 4-(4-nitrophenoxy)piperidine-1-carboxylate (4.5 g, 14.0 mmol, 1.0 equiv) and HCl in 1,4-dioxane (4 N, 20 mL) was stirred for 1 hour. The resulting mixture was concentrated under reduced pressure to afford crude 4-(4-nitrophenoxy)piperidine (3.4 g, crude) as a yellow solid that was used without further purification. LCMS (ESI, m/z): 223.10 [M+H]⁺.

### Step C

A solution of 4-(4-nitrophenoxy)piperidine (2.0 g, 9.0 mmol, 1.0 equiv), 4-(hydroxymethyl)cyclohexan-1-one (3.46 g, 27.0 mmol, 3.0 equiv), NaBH₃CN (1.13 g, 18.0 mmol, 2.0 equiv) in DCE (15 mL) was stirred for 3 hours. The resulting mixture was concentrated under reduced pressure. The residue was purified by silica gel column chromatography, eluting with PE/EtOAc (8:17) to afford the product as a yellow oil that was further purified by reverse phase flash chromatography with the following conditions: column, C18 silica gel; mobile phase, MeCN in water (10 mmol/L NH₄HCO₃), 10% to 50% gradient in 10 min; detector, UV 254 nm. This afforded (4-(4-(4-nitrophenoxy)piperidin-1-yl)cyclohexyl)methanol (750 mg, 25%) as a yellow oil. LCMS (ESI, m/z): 335.19 [M+H]⁺.

### Step D

A solution of (4-(4-(4-nitrophenoxy)piperidin-1-yl)cyclohexyl)methanol (700 mg, 2.09 mmol, 1.0 equiv), 7-bromo-5-fluoro-2-(((tetrahydro-2H-pyran-4-yl)thio)methyl)quinazolin-4(3H)-one (859 mg, 2.30 mmol, 1.1 equiv), [Pd(allyl)Cl]₂ (76.6 mg, 0.21 mmol, 0.1 equiv), t-BuBrettPhos (102 mg, 0.21 mmol, 0.1 equiv), and Cs₂CO₃ (1364 mg, 4.19 mmol, 2.0 equiv) in toluene (6 mL) was stirred overnight at 80° C. The reaction was diluted with water (5 mL) and the aqueous layer was extracted with EtOAc (20×5 mL). The combined organic layers were concentrated and the resulting residue was purified by reverse-phase flash chromatography with the following conditions: column, C18 silica gel; mobile phase, MeCN in water (10 mmol/L NH₄HCO₃), 10% to 90% gradient in 20 min; detector, UV 254 nm to give 5-fluoro-7-((4-(4-(4-nitrophenoxy)piperidin-1-yl)cyclohexyl)methoxy)-2-(((tetrahydro-2H-pyran-4-yl)thio)methyl)quinazolin-4(3H)-one (380 mg, 29%) as a yellow solid. LCMS (ESI, m/z): 627.25 [M+H]⁺.

### Step E

A solution of Fe (425 mg, 7.62 mmol, 13 equiv) and NH₄Cl (63 mg, 1.2 mmol, 2.0 equiv) in EtOH (3 mL) and water (1 mL) was stirred for 10 min at 80° C. followed by the addition of 5-fluoro-7-((4-(4-(4-nitrophenoxy)piperidin-1-yl)cyclohexyl)methoxy)-2-(((tetrahydro-2H-pyran-4-yl)thio)methyl)quinazolin-4(3H)-one (367 mg, 0.59 mmol, 1.0 equiv) in portions at 80° C. After 30 minutes the insoluble solids were filtered off and the filter cake was washed with EtOH (30 mL). The filtrate was concentrated and the residue was purified by reverse phase flash chromatography with the following conditions: column, C18 silica gel; mobile phase, MeCN in water (10 mmol/L NH₄HCO₃), 10% to 60% gradient in 15 min; detector, UV 254 nm. This afforded 7-((4-(4-(4-aminophenoxy)piperidin-1-yl)cyclohexyl)methoxy)-5-fluoro-2-(((tetrahydro-2H-pyran-4-yl)thio)methyl)quinazolin-4(3H)-one (330 mg, 94%) as a yellow solid. LCMS (ESI, m/z): 597.25 [M+H]⁺.

### Step F

A solution of 7-((4-(4-(4-aminophenoxy)piperidin-1-yl)cyclohexyl)methoxy)-5-fluoro-2-(((tetrahydro-2H-pyran-4-yl)thio)methyl)quinazolin-4(3H)-one (320 mg, 0.54 mmol, 1.0 equiv), 3-bromopiperidine-2,6-dione (103 mg, 0.54 mmol, 1.0 equiv), NaHCO₃ (90 mg, 1.1 mmol, 2.0 equiv) in ACN (4 mL) was stirred for 2 days at 90° C. The reaction was diluted with water and extracted with EtOAc (4×20 mL). The combined organic layers were dried and concentrated and the residue was purified by reverse phase flash chromatography with the following conditions: column, C18 silica gel; mobile phase, MeCN in water (10 mmol/L NH₄HCO₃), 10% to 60% gradient in 15 min; detector, UV 254 nm. The crude product (300 mg) was purified by prep-HPLC with the following conditions (Column: Xselect CSH F-Phenyl OBD column, 19*250 mm, 5 μm; Mobile Phase A: water (0.1% FA), Mobile Phase B: ACN; Flow rate: 20 mL/min; Gradient: 20% B to 25% B in 11 min, 25% B; Wave Length: 254 nm; RT1 (min): 10.98) to afford 3-((4-((1-(4-(((5-fluoro-4-oxo-2-(((tetrahydro-2H-pyran-4-yl)thio)methyl)-3,4-dihydroquinazolin-7-yl)oxy)methyl)cyclohexyl) piperidin-4-yl)oxy)phenyl)amino)piperidine-2,6-dione (156 mg, 41%) as an off-white solid. LCMS (ESI, m/z): 708.15 [M+H]⁺. ¹H NMR (300 MHz, DMSO-d₆) δ 12.16 (s, 1H), 10.74 (s, 1H), 6.99-6.83 (m, 2H), 6.79 (dd, J=9.0, 2.2 Hz, 2H), 6.64 (d, J=8.2 Hz, 2H), 5.51 (d, J=7.0 Hz, 1H), 4.40-4.29 (m, 1H), 4.28-4.17 (m, 1H), 4.16-4.09 (m, 1H), 4.01-3.92 (m, 1H), 3.88-3.78 (m, 3H), 3.65-3.60 (m, 3H), 3.24-3.21 (m, 1H), 3.14-2.99 (m, 5H), 2.81-2.66 (m, 1H), 2.65-2.54 (m, 1H), 2.20-1.98 (m, 5H), 1.98-1.66 (m, 9H), 1.65-1.37 (m, 4H), 1.23-1.19 (m, 1H).

### Example 88: Synthesis of 3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione

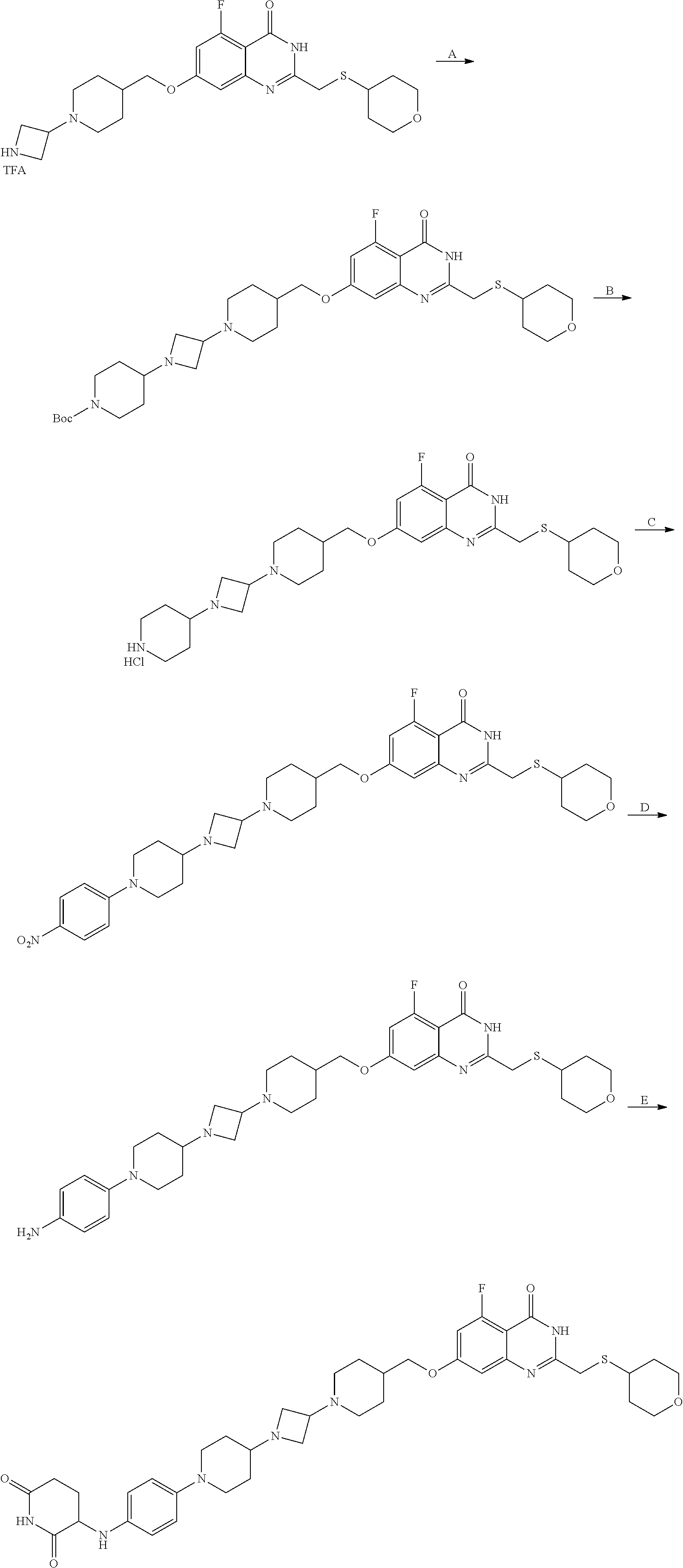

### Step A

A mixture 7-((1-(azetidin-3-yl)piperidin-4-yl)methoxy)-5-fluoro-2-(((tetrahydro-2H-pyran-4-yl)thio)methyl)quinazolin-4(3H)-one-TFA (5.0 g, 8.92 mmol, 1.0 equiv), tert-butyl 4-oxopiperidine-1-carboxylate (4.44 g, 22.3 mmol, 2.5 equiv) and STAB (6.62 g, 31.2 mmol, 3.5 equiv) in DCE (200 mL) was stirred overnight. After concentration, the crude product was purified by silica gel chromatography eluting with CHCl₃/MeOH (87:13) to afford tert-butyl 4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidine-1-carboxylate (4 g, 69%) as a yellow solid. LCMS (ESI, m/z): 646.35 [M+H]0.

### Step B

A mixture of tert-butyl 4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidine-1-carboxylate (3.94 g, 6.10 mmol, 1.0 equiv) in HCl in 1,4-dioxane (50 mL, 4 M) was stirred for 3 hours. The resulting mixture was concentrated under reduced pressure to afford 5-fluoro-7-((1-(1-(piperidin-4-yl)azetidin-3-yl)piperidin-4-yl)methoxy)-2-(((tetrahydro-2H-pyran-4-yl)thio)methyl)quinazolin-4(3H)-one hydrochloride (3.93 g) as a crude white solid. The crude product was used in the next step without further purification. LCMS (ESI, m/z): 546.30 [M+H]⁺.

### Step C

A mixture of 5-fluoro-7-((1-(1-(piperidin-4-yl)azetidin-3-yl)piperidin-4-yl)methoxy)-2-(((tetrahydro-2H-pyran-4-yl)thio)methyl)quinazolin-4(3H)-one hydrochloride (3.93 g, 6.74 mmol, 1.0 equiv), 4-fluoronitrobenzene (1.05 g, 7.42 mmol, 1.1 equiv) and DIEA (4.36 g, 33.7 mmol, 5.0 equiv) in NMP (20 mL) was stirred overnight at 80° C. The mixture was allowed to cool down to room temperature. The residue was purified by C18 reverse phase chromatography eluting with water (10 mmol/L NH₄HCO₃)/CH₃CN (45:55) to afford 5-fluoro-7-((1-(1-(1-(4-nitrophenyl)piperidin-4-yl)azetidin-3-yl)piperidin-4-yl)methoxy)-2-(((tetrahydro-2H-pyran-4-yl)thio)methyl)quinazolin-4(3H)-one (1.46 g, 33%) as a yellow solid. LCMS (ESI, m/z): 667.35 [M+H]⁺.

### Step D

To a stirred mixture of 5-fluoro-7-((1-(1-(1-(4-nitrophenyl)piperidin-4-yl)azetidin-3-yl)piperidin-4-yl)methoxy)-2-(((tetrahydro-2H-pyran-4-yl)thio)methyl)quinazolin-4(3H)-one (1.46 g, 2.19 mmol, 1.0 equiv) and NH₄Cl (352 mg, 6.57 mmol, 3.0 equiv) in EtOH (55 mL) and water (10 mL) was added Fe (857 mg, 15.3 mmol, 7.0 equiv) in portions at 80° C. The resulting mixture was stirred for 4.5 hours at 80° C. The mixture was filtered and the filter cake was washed with EtOH (5×70 mL). The filtrate was concentrated under reduced pressure. After concentration, the residue was purified by C18 reverse phase chromatography eluting with water (10 mmol/L NH₄HCO₃)/CH₃CN (58:42) to afford 7-((1-(1-(1-(4-aminophenyl)piperidin-4-yl)azetidin-3-yl)piperidin-4-yl)methoxy)-5-fluoro-2-(((tetrahydro-2H-pyran-4-yl)thio)methyl)quinazolin-4(3H)-one (1.21 g, 76%) as a grey solid. LCMS (ESI, m/z): 637.35 [M+H]⁺.

### Step E

A mixture of 7-((1-(1-(1-(4-aminophenyl)piperidin-4-yl)azetidin-3-yl)piperidin-4-yl)methoxy)-5-fluoro-2-(((tetrahydro-2H-pyran-4-yl)thio)methyl)quinazolin-4(3H)-one (200 mg, 0.31 mmol, 1.0 equiv), 3-bromopiperidine-2,6-dione (302 mg, 1.57 mmol, 5.0 equiv) and NaHCO₃ (185 mg, 2.20 mmol, 7.0 equiv) in ACN (30 mL) was stirred for 5 days at 90° C. The resulting mixture was filtered and the filter cake was washed with DCM (3×100 mL). The filtrate was concentrated under reduced pressure. The crude product (150 mg) was purified by prep-HPLC with the following conditions (Column: Xselect CSH C18 OBD Column 30*150 mm 5 μm; Mobile Phase A: water (10 mmol/L NH₄HCO₃), Mobile Phase B: ACN; Flow rate: 60 mL/min; Gradient: 22% B to 32% B in 10 min, 32% B to 32% B in 12 min, 32% B; Wave Length: 254/220 nm; RT1 (min): 11.580) to afford 3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione (55 mg, 23%) as a grey solid. LCMS (ESI, m/z): 748.25 [M+H]⁺. ¹H NMR (400 MHz, DMSO-d₆) δ 12.14 (s, 1H), 10.74 (s, 1H), 6.93-6.84 (m, 2H), 6.75 (d, J=8.0 Hz, 2H), 6.60 (d, J=8.2 Hz, 2H), 5.35 (d, J=7.2 Hz, 1H), 4.25-4.12 (m, 1H), 3.98 (d, J=5.8 Hz, 2H), 3.89-3.75 (m, 2H), 3.62 (s, 2H), 3.42-3.35 (m, 3H), 3.29-3.20 (m, 3H), 3.10-3.02 (m, 1H), 2.84-2.66 (m, 6H), 2.62-2.57 (m, 1H), 2.57-2.54 (m, 1H), 2.15-2.00 (m, 2H), 1.93-1.81 (m, 3H), 1.80-1.65 (m, 7H), 1.51-1.40 (m, 2H), 1.39-1.20 (m, 5H).

Example 89 was synthesized according to the procedure described for the synthesis 3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione (Example 88) using appropriate building blocks and modified reaction conditions (such as reagents, reagent ratio, temperature, and reaction time) and purification conditions as needed.

| Ex- | | |
|---|---|---|
| am- | | Analytical |
| ple | Structure | Data |
| 89 | 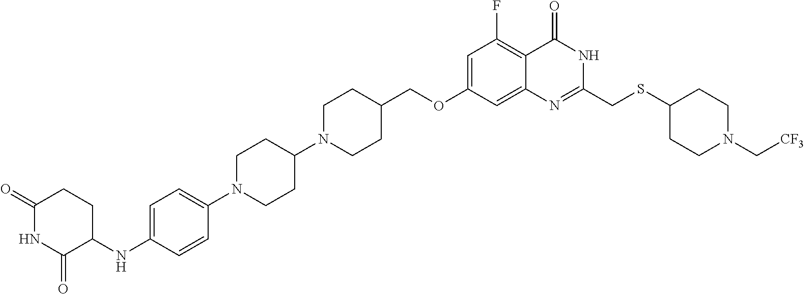 [SMILES: O=C1CCC(Nc2ccc(N3CCC(N4CCC(COc5cc(F)c6c(=O)[nH]c(CSC7CCN(CC(F)(F)F)CC7)nc6c5)CC4)CC3)cc2)C(=O)N1] | LCMS (ESI, m/z): 774.15 [M + H]⁺; 1H NMR (300 MHz, DMSO- d6) δ 12.10 (s, 1H), 10.73 (s, 1H), 8.21 (s, 1H) HCOOH, 6.94-6.85 (m, 2H), 6.76 (d, J = |
| | 3-((4-(4-(((5-fluoro-4-oxo-2-(((1-(2,2,2- | 8.9 Hz, |
| | trifluoroethyl)piperidin-4-yl)thio)methyl)-3,4- | 2H), 6.60 |
| | dihydroquinazolin-7-yl)oxy)methyl)-[1,4′- | (d, |
| | bipiperidin]-1′-yl)phenyl)amino)piperidine-2,6- | J = 8.9 |
| | dione | Hz, 2H), |
| | | 5.341-5.23 |
| | | (m, 1H), |
| | | 4.18 |
| | | (d, J = 10.9 |
| | | Hz, 2H), |
| | | 3.98 |
| | | (s, J = 5.4 |
| | | Hz, 2H), |
| | | 3.48- |
| | | 3.38 (m, |
| | | 5H), 3.20- |
| | | 3.05 (m, |
| | | 2H), |
| | | 2.97 (d, J = |
| | | 10.3 Hz, |
| | | 2H), |
| | | 2.91- |
| | | 2.81 (m, |
| | | 3H), 2.78- |
| | | 2.60 (m, |
| | | 2H), |
| | | 2.48-2.35 |
| | | (m, 3H), |
| | | 2.30-2.03 |
| | | (m, 3H), |
| | | 1.98-1.73 |
| | | (m, 8H), |
| | | 1.68-1.20 |
| | | (m, 6H). |

### Example 90: Synthesis of 3-((4-(3-((4-(((5-fluoro-2-((((1r,4r)-4-hydroxycyclohexyl)thio)methyl)-4-oxo-3,4-dihydroquinazolin-7-yl)oxy)methyl)piperidin-1-yl)methyl)cyclobutoxy)phenyl)amino)piperidine-2,6-dione formate

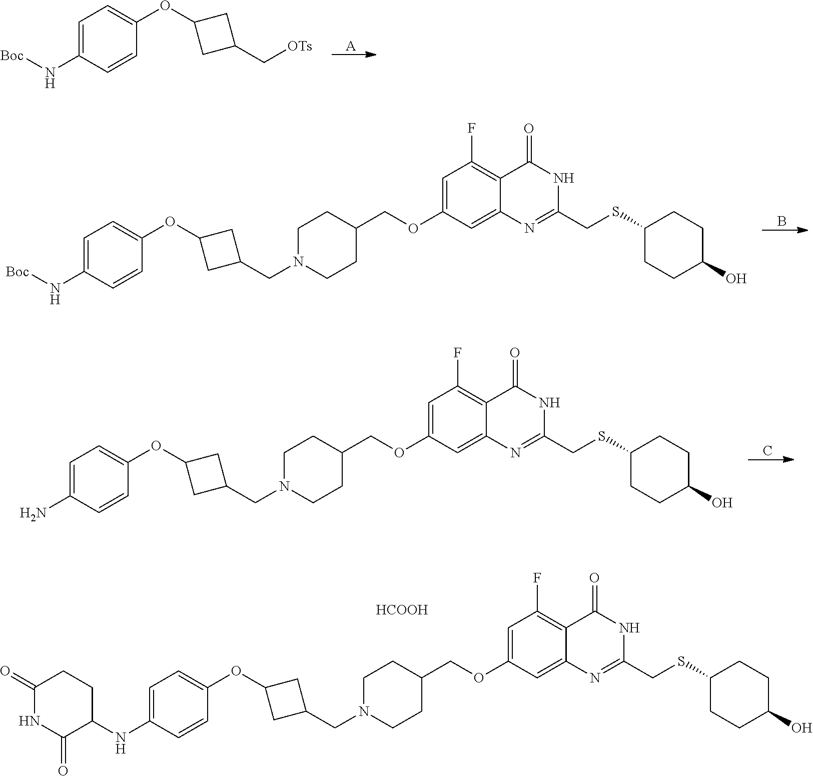

### Step A

A solution of 5-fluoro-2-((((1r,4r)-4-hydroxycyclohexyl)thio)methyl)-7-(piperidin-4-ylmethoxy)quinazolin-4(3H)-one (607 mg, 1.44 mmol, 1.0 equiv), (3-(4-((tert-butoxycarbonyl)amino)phenoxy)cyclobutyl)methyl 4-methylbenzenesulfonate (644 mg, 1.44 mmol, 1.0 equiv), KI (24 mg, 0.14 mmol, 0.1 equiv) and DIEA (558 mg, 4.32 mmol, 3.0 equiv) in ACN (20 mL) was stirred overnight at 60° C. The resulting mixture was concentrated under reduced pressure. The residue was purified by silica gel column chromatography, eluting with CH₂Cl₂/MeOH (82:18) to afford tert-butyl (4-(3-((4-(((5-fluoro-2-((((1r,4r)-4-hydroxycyclohexyl)thio)methyl)-4-oxo-3,4-dihydroquinazolin-7-yl)oxy)methyl)piperidin-1-yl)methyl)cyclobutoxy)phenyl)carbamate (680 mg, 68%) as a white solid. LCMS (ESI, m/z): 697.30 [M+H]⁺.

### Step B

A solution of tert-butyl (4-(3-((4-(((5-fluoro-2-((((1r,4r)-4-hydroxycyclohexyl)thio)methyl)-4-oxo-3,4-dihydroquinazolin-7-yl)oxy)methyl)piperidin-1-yl)methyl)cyclobutoxy)phenyl)carbamate (680 mg, 0.98 mmol, 1.0 equiv) in HCl in 1,4-dioxane (20 mL, 4M) as stirred for 1 hour at room temperature. The resulting mixture was concentrated under reduced pressure to give 7-((1-((3-(4-aminophenoxy)cyclobutyl)methyl)piperidin-4-yl)methoxy)-5-fluoro-2-((((1r,4r)-4-hydroxycyclohexyl)thio)methyl)quinazolin-4(3H)-one (625 mg) as a yellow crude solid. The crude product was used without further purification. LCMS (ESI, m/z): 597.30[M+H]⁺.

### Step C

A solution of 7-((1-((3-(4-aminophenoxy)cyclobutyl)methyl)piperidin-4-yl)methoxy)-5-fluoro-2-((((1r,4r)-4-hydroxycyclohexyl)thio)methyl)quinazolin-4(3H)-one (140 mg, 0.23 mmol, 1.0 equiv), 3-bromopiperidine-2,6-dione (135 mg, 0.705 mmol, 3.0 equiv) and NaHCO₃ (99 mg, 1.2 mmol, 5 equiv) in ACN (15 mL) was stirred overnight at 90° C. The resulting mixture was concentrated under reduced pressure. The residue was purified by reverse-phase flash chromatography with the following conditions: column, C18 silica gel; mobile phase, MeCN in water (10 mmol/L NH₄HCO₃), 5% to 13% gradient in 15 min; detector, UV 254 nm. The resulting product was further purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 20% B in 10 min, 20% B; Wave Length: 254/220 nm; RT1 (min): 9.25) to afford 3-((4-(3-((4-(((5-fluoro-2-((((1r,4r)-4-hydroxycyclohexyl)thio)methyl)-4-oxo-3,4-dihydroquinazolin-7-yl)oxy)methyl)piperidin-1-yl)methyl)cyclobutoxy)phenyl)amino)piperidine-2,6-dione formate (47 mg, 28%) as a grey solid. LCMS (ESI, m/z): 708.30 [M+H]⁺; 1H NMR (400 MHz, DMSO-d6) δ 12.10 (s, 1H), 10.75 (s, 1H), 8.21 (s, 1H), 6.87 (d, J=9.8 Hz, 2H), 6.61 (s, 4H), 5.41 (s, 1H), 4.73-4.57 (m, 1H), 4.24-4.11 (m, 1H), 3.98 (d, J=5.9 Hz, 2H), 3.55-3.50 (m, 5H), 2.92 (d, J=10.9 Hz, 2H), 2.78-2.76 (m, 2H), 2.63-2.50 (m, 3H), 2.21-1.90 (m, 9H), 1.87-1.69 (m, 6H), 1.38-1.08 (m, 6H).

Examples 91-108 were synthesized according to the procedure described for the synthesis 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione (Example 48) using appropriate building blocks and modified reaction conditions (such as reagents, reagent ratio, temperature, and reaction time) and purification conditions as needed.

| Ex- | | |
|---|---|---|
| am- | | Analytical |
| ple | Structure | Data |
| 91 | 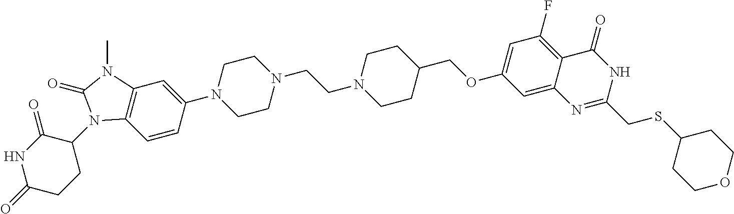 [SMILES: Cn1c(=O)n(C2CCC(=O)NC2=O)c2ccc(N3CCN(CCN4CCC(COc5cc(F)c6c(=O)[nH]c(CSC7CCOCC7)nc6c5)CC4)CC3)cc21] | LCMS (ESI, m/z): 777.30 [M + H]⁺; ¹H NMR (300 MHz, DMSO-d₆) δ 12.22 (s, 1H), 11.04 (s, 1H), 6.99- 6.81 (m, 4H), 6.63 (dd, |
| | 3-(5-(4-(2-(4-(((5-fluoro-4-oxo-2-(((tetrahydro-2H-pyran-4- | J = 8.7, 2.2 |
| | yl)thio)methyl)-3,4-dihydroquinazolin-7- | Hz, 1H), |
| | yl)oxy)methyl)piperidin-1-yl)ethyl)piperazin-1-yl)-3- | 5.30 (dd, |
| | methyl-2-oxo-2,3-dihydro-1H-benzo[d]imidazol-1- | J = 12.7, |
| | yl)piperidine-2,6-dione | 5.3 Hz, |
| | | 1H), 4.02 |
| | | (d, J = 6.1 |
| | | Hz, 2H), |
| | | 3.88-3.76 |
| | | (m, 2H), |
| | | 3.63 (s, |
| | | 2H), 3.41- |
| | | 3.26 (m, |
| | | 8H), 3.16- |
| | | 3.02 (m, |
| | | 6H), 2.94- |
| | | 2.81 (m, |
| | | 2H), 2.75- |
| | | 2.58 (m, |
| | | 8H), 2.03- |
| | | 1.79 (m, |
| | | 7H), 1.61- |
| | | 1.37 (m, 4H). |
| 92 | 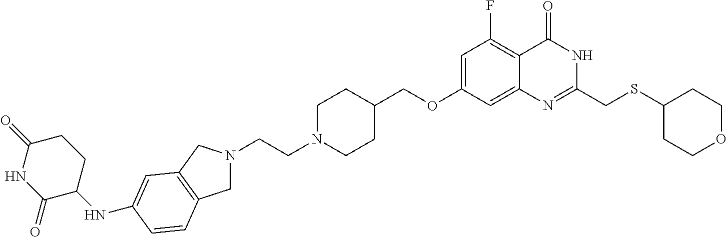 [SMILES: O=C1CCC(Nc2ccc3c(c2)CN(CCN2CCC(COc4cc(F)c5c(=O)[nH]c(CSC6CCOCC6)nc5c4)CC2)C3)C(=O)N1] | LCMS (ESI, m/z): 679.20 [M + H]⁺; ¹H NMR (400 MHz, DMSO-d₆) δ 12.10 (s, 1H), 10.80 (s, 1H), 6.99- 6.85 (m, 3H), 6.62- 6.48 (m, |
| | 3-((2-(2-(4-(((5-fluoro-4-oxo-2-(((tetrahydro-2H-pyran-4- | 2H), 5.68 |
| | yl)thio)methyl)-3,4-dihydroquinazolin-7- | (d, J = 7.6 |
| | yl)oxy)methyl)piperidin-1-yl)ethyl)isoindolin-5- | Hz, 1H), |
| | yl)amino)piperidine-2,6-dione | 4.33- |
| | | 4.25 (m, |
| | | 1H), 3.98 (d, |
| | | J = 5.9 Hz, |
| | | 2H), 3.89- |
| | | 3.60 (m, |
| | | 9H), 3.12- |
| | | 3.00 (m, |
| | | 1H), 2.94- |
| | | 2.85 (m, |
| | | 2H), |
| | | 2.80-2.68 |
| | | (m, 3H), |
| | | 2.58- |
| | | 2.50 (m, |
| | | 2H), 2.47- |
| | | 2.42 (m, |
| | | 1H), |
| | | 2.18- |
| | | 2.05 (m, |
| | | 1H), |
| | | 2.00-1.70 |
| | | (m, 8H), |
| | | 1.52- |
| | | 1.20 (m, 5H). |
| 93 | 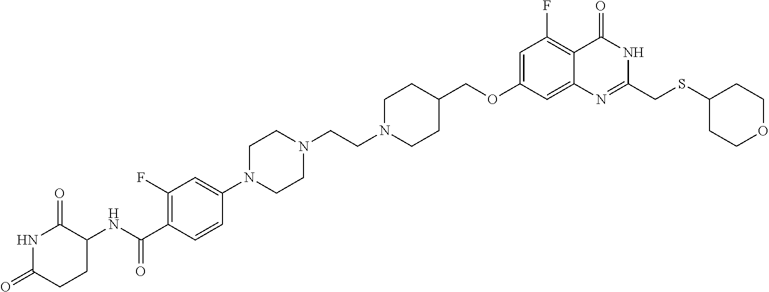 [SMILES: O=C1CCC(NC(=O)c2ccc(N3CCN(CCN4CCC(COc5cc(F)c6c(=O)[nH]c(CSC7CCOCC7)nc6c5)CC4)CC3)cc2F)C(=O)N1] | LCMS (ESI, m/z): 768.25 [M + H]⁺; ¹H NMR (400 MHz, DMSO-d₆) δ 12.16 (s, 1H), 10.84 (s, 1H), 8.04 (t, J = 7.2 Hz, 1H), 7.63 (t, J = 9.0 Hz, 1H), 7.00- 6.63 (m, 4H), 4.79- 4.68 (m, |
| | N-(2,6-dioxopiperidin-3-yl)-2-fluoro-4-(4-(2-(4-(((5-fluoro- | 1H), 3.97 (d, |
| | 4-oxo-2-(((tetrahydro-2H-pyran-4-yl)thio)methyl)-3,4- | J = 5.9 Hz, |
| | dihydroquinazolin-7-yl)oxy)methyl)piperidin-1- | 2H), 3.85- |
| | yl)ethyl)piperazin-1-yl)benzamide | 3.75 (m, |
| | | 2H), 3.65 |
| | | (s, 2H), 3.55- |
| | | 3.40 (m, |
| | | 1H), 3.37- |
| | | 3.32 (m, 2H), |
| | | 3.29-3.25 (m, |
| | | 4H), |
| | | 3.10-3.01 |
| | | (m, 1H), |
| | | 2.99- |
| | | 2.89 (m, |
| | | 2H), 2.83- |
| | | 2.71 (m, 1H), |
| | | 2.59-2.52 (m, |
| | | 4H), |
| | | 2.49-2.45 |
| | | (m, 4H), 2.20- |
| | | 2.08 (m, |
| | | 1H), 2.05- |
| | | 1.85 (m, |
| | | 5H), 1.80- |
| | | 1.65 (m, 3H), |
| | | 1.52-1.38 (m, |
| | | 2H), |
| | | 1.38-1.21 |
| | | (m, 2H) |
| 94 | 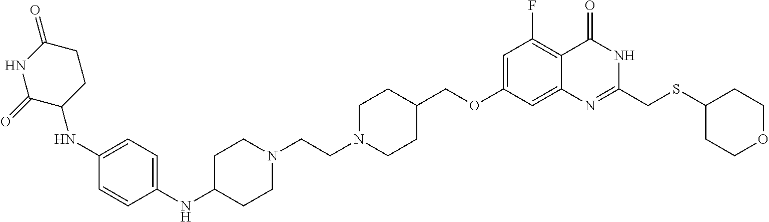 [SMILES: O=C1CCC(Nc2ccc(NC3CCN(CCN4CCC(COc5cc(F)c6c(=O)[nH]c(CSC7CCOCC7)nc6c5)CC4)CC3)cc2)C(=O)N1] | LCMS (ESI, m/z): 736.55 [M + H]⁺; ¹H NMR (400 MHz, DMSO-d₆) δ 12.15 (br, 1H), 10.71 (s, 1H), 8.19 (s, 2H) HCOOH, 6.91- |
| | 3-((4-((1-(2-(4-(((5-fluoro-4-oxo-2-(((tetrahydro-2H-pyran- | 6.85 (m, |
| | 4-yl)thio)methyl)-3,4-dihydroquinazolin-7- | 2H), 6.54 |
| | yl)oxy)methyl)piperidin-1-yl)ethyl)piperidin-4- | (d, J = 8.8 |
| | yl)amino)phenyl)amino)piperidine-2,6-dione | Hz, 2H), |
| | | 6.4 (d, J = |
| | | 8.8 Hz, |
| | | 2H), 5.00 |
| | | (br, 1H), |
| | | 4.08-4.04 (m, |
| | | 1H), 4.02- |
| | | 3.95 |
| | | (m, 2H), |
| | | 3.88- |
| | | 3.79 (m, |
| | | 2H), 3.62 |
| | | (s, 3H), |
| | | 3.38-3.28 |
| | | (m, 3H), 3.08- |
| | | 3.04 (m, |
| | | 2H), 3.02- |
| | | 2.88 (m, 4H), |
| | | 2.80-2.69 (m, |
| | | 1H), |
| | | 2.65-2.55 |
| | | (m, 4H), 2.18- |
| | | 2.09(m, |
| | | 3H), 2.07- |
| | | 1.97 (m, |
| | | 2H), 1.95- |
| | | 1.74 (m, 8H), |
| | | 1.55-1.40 |
| | | (m, 2H), |
| | | 1.40-1.22 (m, |
| | | 4H). |
| 95 | 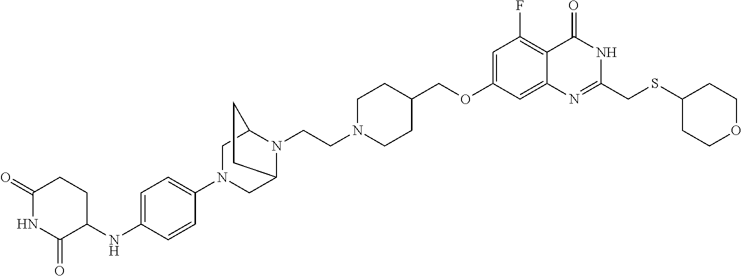 [SMILES: O=C1CCC(Nc2ccc(N3CC4CCC(C3)N4CCN3CCC(COc4cc(F)c5c(=O)[nH]c(CSC6CCOCC6)nc5c4)CC3)cc2)C(=O)N1] | LCMS (ESI, m/z): 748.40 [M + H]⁺; ¹H NMR (300 MHz, DMSO-d₆) δ 10.72 (s, 1H), 8.21 (s, 1H), 6.86 (d, J = 12 Hz, 2H), 6.70-6.50 (m, 4H), 5.22 (br, 1H), 4.29- 4.09 |
| | 3-((4-(8-(2-(4-(((5-fluoro-4-oxo-2-(((tetrahydro-2H-pyran- | (m, 1H), |
| | 4-yl)thio)methyl)-3,4-dihydroquinazolin-7- | 3.98 (d, J = |
| | yl)oxy)methyl)piperidin-1-yl)ethyl)-3,8- | 5.8 Hz, |
| | diazabicyclo[3.2.1]octan-3-yl)phenyl)amino)piperidine-2,6- | 2H), 3.85- |
| | dione | 3.76 (m, 3H), |
| | | 3.72-3.68 (m, |
| | | 2H), 3.47- |
| | | 3.44 |
| | | (m, 2H), 3.40- |
| | | 3.27 (m, |
| | | 5H), 3.21- |
| | | 3.13 (m, 2H), |
| | | 3.08-2.95 (m, |
| | | 3H), 2.78- |
| | | 2.66 |
| | | (m, 3H), 2.65- |
| | | 2.58 (m, |
| | | 1H), 2.19- |
| | | 2.00 (m, 3H), |
| | | 1.95-1.83 (m, |
| | | 4H), 1.81-1.62 |
| | | (m, 5H), |
| | | 1.57-1.19 |
| | | (m, 5H) |
| 96 | 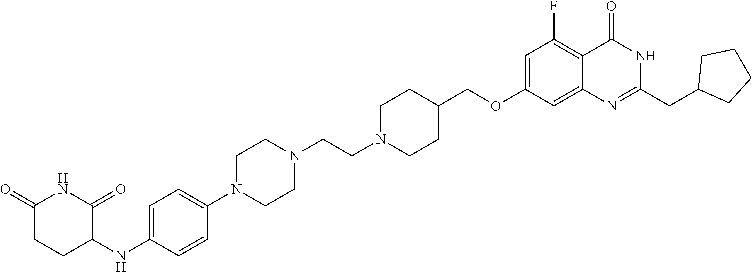 [SMILES: O=C1CCC(Nc2ccc(N3CCN(CCN4CCC(COc5cc(F)c6c(=O)[nH]c(CC7CCCC7)nc6c5)CC4)CC3)cc2)C(=O)N1] | LCMS (ESI, m/z): 674.30 [M + H]⁺; ¹H NMR (400 MHz, DMSO-d₆) δ 12.00 (br, 1H), 10.76 (s, 1H), 6.87 (d, J = 2.4 Hz, 1H), 6.82 (dd, J = 12.6, 2.4 Hz, 1H), 6.74 (d, |
| | 3-((4-(4-(2-(4-(((2-(cyclopentylmethyl)-5-fluoro-4-oxo-3,4- | J = 8.7 Hz, |
| | dihydroquinazolin-7-yl)oxy)methyl)piperidin-1- | 2H), 6.61 |
| | yl)ethyl)piperazin-1-yl)phenyl)amino)piperidine-2,6-dione | (d, J = 8.7 |
| | | Hz, 2H), |
| | | 5.35 (d, J = |
| | | 7.2 Hz, |
| | | 1H), 4.22- |
| | | 4.16 (m, |
| | | 1H), 3.96 (d, |
| | | J = 5.9 Hz, |
| | | 2H), 2.99- |
| | | 2.88 (m, |
| | | 6H), 2.79- |
| | | 2.68 (m, |
| | | 1H), 2.64- |
| | | 2.54 (m, 1H), |
| | | 2.53-2.48 |
| | | (m, 5H), |
| | | 2.46-2.40 |
| | | (m, 4H), |
| | | 2.38-2.26 |
| | | (m, 1H), |
| | | 2.17-2.06 |
| | | (m, 1H), |
| | | 2.00-1.90 (m, |
| | | 2H), 1.89- |
| | | 1.80 |
| | | (m, 1H), 1.77- |
| | | 1.65 (m, |
| | | 5H), 1.68- |
| | | 1.59 (m, 2H), |
| | | 1.59-1.49 |
| | | (m, 2H), |
| | | 1.36-1.15 |
| | | (m, 5H) |
| 97 | 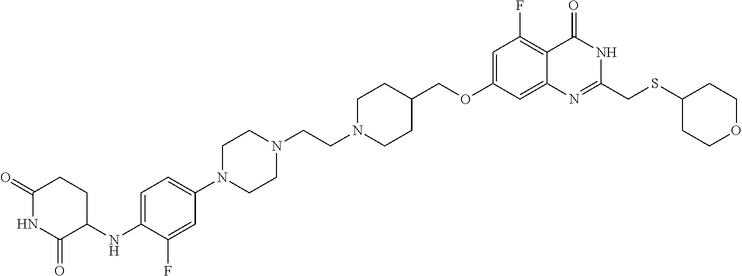 [SMILES: O=C1CCC(Nc2ccc(N3CCN(CCN4CCC(COc5cc(F)c6c(=O)[nH]c(CSC7CCOCC7)nc6c5)CC4)CC3)cc2F)C(=O)N1] | LCMS (ESI, m/z): 740.40 [M + H]⁺; ¹H NMR (300 MHz, DMSO-d₆) δ12.10 (br, 1H), 10.79 (br, 1H), 6.93- 6.82 (m, 2H), 6.81- 6.68 (m, 2H), 6.58 (dd, J = 8.8, |
| | 3-((2-fluoro-4-(4-(2-(4-(((5-fluoro-4-oxo-2-(((tetrahydro- | 2.5 Hz, |
| | 2H-pyran-4-yl)thio)methyl)-3,4-dihydroquinazolin-7- | 1H), 5.10 |
| | yl)oxy)methyl)piperidin-1-yl)ethyl)piperazin-1- | (d, J = 6.3 Hz, |
| | yl)phenyl)amino)piperidine-2,6-dione | 1H), 4.33- |
| | | 4.15 (m, |
| | | 1H), 3.98 |
| | | (d, J = 5.8 |
| | | Hz, 2H), |
| | | 3.90-3.70 (m, |
| | | 2H), 3.62 |
| | | (s, 2H), |
| | | 3.40-3.35 (m, |
| | | 1H), 3.28- |
| | | 3.20 |
| | | (m, 2H), 3.15- |
| | | 2.88 (m, |
| | | 6H), 2.85- |
| | | 2.68 (m, |
| | | 1H), 2.62- |
| | | 2.55 (m, |
| | | 3H), 2.48- |
| | | 2.36 (m, 5H), |
| | | 2.18-2.10 (m, |
| | | 1H), 2.05- |
| | | 1.85 (m, |
| | | 5H), 1.84- |
| | | 1.65 (m, |
| | | 3H), 1.55- |
| | | 1.20 |
| | | (m, 4H). |
| 98 | 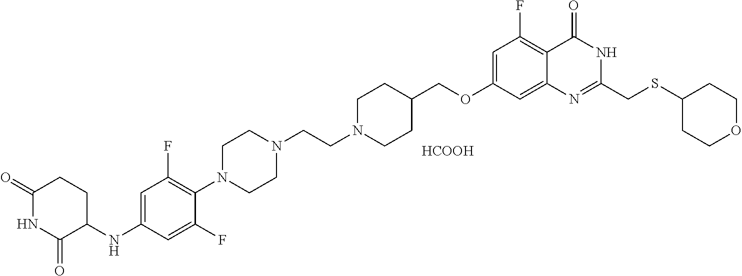 [SMILES: O=C1CCC(Nc2cc(F)c(N3CCN(CCN4CCC(COc5cc(F)c6c(=O)[nH]c(CSC7CCOCC7)nc6c5)CC4)CC3)c(F)c2)C(=O)N1 ; "HCOOH"] | LCMS (ESI, m/z): 758.45 [M + H]⁺; ¹H NMR (300 MHz, DMSO-d₆) δ 12.14 (br, 1H), 10.80 (s, 1H), 8.19 (s, 1H), 7.03-6.77 (m, 2H), 6.35 (s, 1H), 6.30 (s, 1H), 6.24(d, J = 7.5 Hz, |
| | 3-((3,5-difluoro-4-(4-(2-(4-(((5-fluoro-4-oxo-2- | 1H), 4.36- |
| | (((tetrahydro-2H-pyran-4-yl)thio)methyl)-3,4- | 4.25 |
| | dihydroquinazolin-7-yl)oxy)methyl)piperidin-1- | (m, 1H), |
| | yl)ethyl)piperazin-1-yl)phenyl)amino)piperidine-2,6-dione | 4.02- |
| | formate | 3.96 (m, |
| | | 2H), 3.88- |
| | | 3.77 (m, 3H), |
| | | 3.62 (s, |
| | | 2H), 3.40- |
| | | 3.28 (m, |
| | | 3H), 3.13- |
| | | 2.91 (m, 7H), |
| | | 2.80-2.55 (m, |
| | | 3H), 2.48- |
| | | 2.44 |
| | | (m, 5H), 2.13- |
| | | 1.97 (m, |
| | | 3H), 1.95- |
| | | 1.70 (m, 6H), |
| | | 1.53-1.23 (m, |
| | | 4H). |
| 99 | 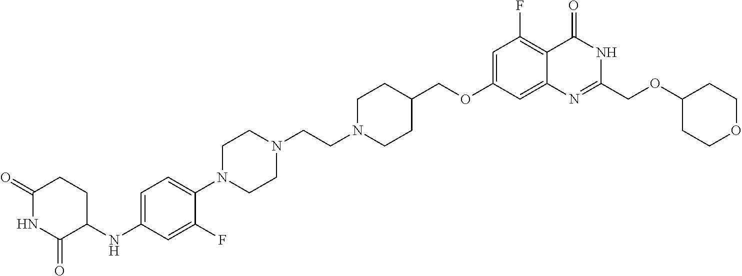 [SMILES: O=C1CCC(Nc2ccc(N3CCN(CCN4CCC(COc5cc(F)c6c(=O)[nH]c(COC7CCOCC7)nc6c5)CC4)CC3)c(F)c2)C(=O)N1] | LCMS (ESI, m/z): 724.20 [M + H]⁺; ¹H NMR (400 MHz, DMSO-d₆) δ 12.01 (s, 1H), 10.77 (s, 1H), 6.95- 6.87 (m, 2H), 6.85- 6.78 (m, 1H), 6.65 (d, J = 15.1, 2.6 Hz, 1H), 6.42 |
| | 3-((3-fluoro-4-(4-(2-(4-(((5-fluoro-4-oxo-2-(((tetrahydro- | (d, J = |
| | 2H-pyran-4-yl)oxy)methyl)-3,4-dihydroquinazolin-7- | 8.5, 2.6 |
| | yl)oxy)methyl)piperidin-1-yl)ethyl)piperazin-1- | Hz, 1H), |
| | yl)phenyl)amino)piperidine-2,6-dione | 5.80 (d, J = |
| | | 7.6 Hz, |
| | | 1H), 4.38 |
| | | (s, 2H), |
| | | 4.31-4.21 (m, |
| | | 1H), 3.98 (d, |
| | | J = 6.0 Hz, |
| | | 2H), 3.87- |
| | | 3.75 (m, |
| | | 2H), 3.70- |
| | | 3.60 (m, |
| | | 1H), 3.39- |
| | | 3.35 (m, 3H), |
| | | 2.97-2.80 (m, |
| | | 6H), 2.80- |
| | | 2.71 (m, |
| | | 2H), 2.60- |
| | | 2.58 |
| | | (m, 1H), 2.49- |
| | | 2.38 (m, |
| | | 4H), 2.13- |
| | | 2.06 (m, 1H), |
| | | 2.01-1.68 (m, |
| | | 9H), 1.59-1.41 |
| | | (m, 2H), 1.35- |
| | | 1.19 (m, 2H). |
| 100 | 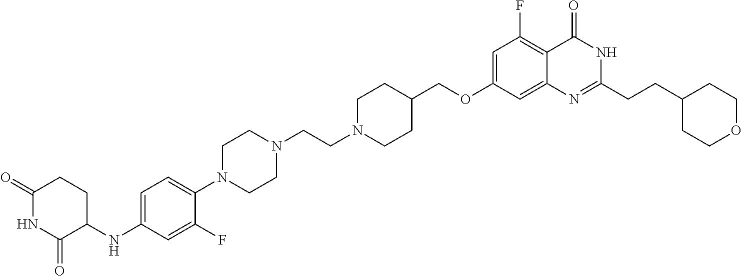 [SMILES: O=C1CCC(Nc2ccc(N3CCN(CCN4CCC(COc5cc(F)c6c(=O)[nH]c(CCC7CCOCC7)nc6c5)CC4)CC3)c(F)c2)C(=O)N1] | LCMS (ESI, m/z): 722.35 [M + H]⁺; ¹H NMR (400 MHz, DMSO-d₆) δ 12.03 (s, 1H), 10.77 (s, 1H), 7.01- 6.70 (m, 3H), 6.53 (d, J = 15.2 Hz, 1H), 6.43 (d, J = 8.0 Hz, 1H), |
| | 3-((3-fluoro-4-(4-(2-(4-(((5-fluoro-4-oxo-2-(2-(tetrahydro- | 5.79 (d, J = |
| | 2H-pyran-4-yl)ethyl)-3,4-dihydroquinazolin-7- | 7.7 Hz, |
| | yl)oxy)methyl)piperidin-1-yl)ethyl)piperazin-1- | 1H), 4.33- |
| | yl)phenyl)amino)piperidine-2,6-dione | 4.20 |
| | | (m, 1H), |
| | | 3.90 (s, |
| | | 2H), 3.88- |
| | | 3.79 |
| | | (m, 2H), |
| | | 3.31- |
| | | 3.19 (m, |
| | | 3H), 3.00- |
| | | 2.68 (m, |
| | | 7H), 2.62- |
| | | 2.53 (m, 5H), |
| | | 2.48-2.40 (m, |
| | | 4H), 2.16- |
| | | 2.05 |
| | | (m, 1H), |
| | | 2.02-1.91 |
| | | (m, 2H), 1.90- |
| | | 1.81 (m, |
| | | 1H), 1.80- |
| | | 1.58 (m, |
| | | 7H), 1.55- |
| | | 1.45 (m, 1H), |
| | | 1.37-1.12 |
| | | (m, 5H |
| 101 | 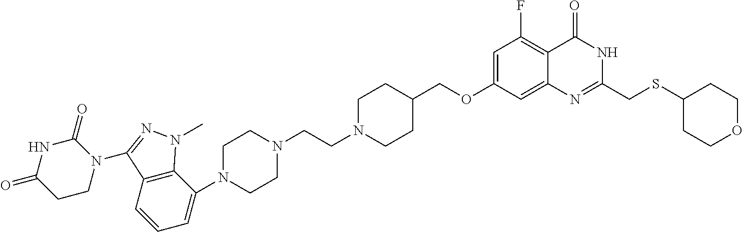 [SMILES: Cn1nc(N2CCC(=O)NC2=O)c2cccc(N3CCN(CCN4CCC(COc5cc(F)c6c(=O)[nH]c(CSC7CCOCC7)nc6c5)CC4)CC3)c21] | LCMS (ESI, m/z): 762.65 [M + H]⁺; ¹HNMR (300 MHz, DMSO-d₆) δ12.15 (s, 1H), 8.23 (s, 1H), 7.32 (dd, J1 = 1.8 Hz, J2 = 7.2 Hz, 1H), 7.05- 6.95 (m, |
| | 1-(7-(4-(2-(4-(((5-fluoro-4-oxo-2-(((tetrahydro-2H-pyran-4- | 2H), 6.90- |
| | yl)thio)methyl)-3,4-dihydroquinazolin-7- | 6.85 (m, 2H), |
| | yl)oxy)methyl)piperidin-1-yl)ethyl)piperazin-1-yl)-1- | 4.28(s, |
| | methyl-1H-indazol-3-yl)dihydropyrimidine-2,4(1H,3H)- | 3H), 4.05- |
| | dione | 3.93 (d, J = |
| | | 5.8 Hz, |
| | | 2H), 3.90- |
| | | 3.76 |
| | | (m, 5H), |
| | | 3.63 (s, |
| | | 2H), 3.40- |
| | | 3.30 (m, |
| | | 5H), 3.13- |
| | | 2.85 |
| | | (m, 6H), |
| | | 2.80- |
| | | 2.69(m, |
| | | 2H), 2.55 |
| | | (s, 3H), 2.12- |
| | | 1.98(m, |
| | | 2H), 1.95- |
| | | 1.84(m, |
| | | 2H), 1.72- |
| | | 1.70 (m, |
| | | 3H), 1.56- |
| | | 1.19 (m, 5H). |
| 102 | 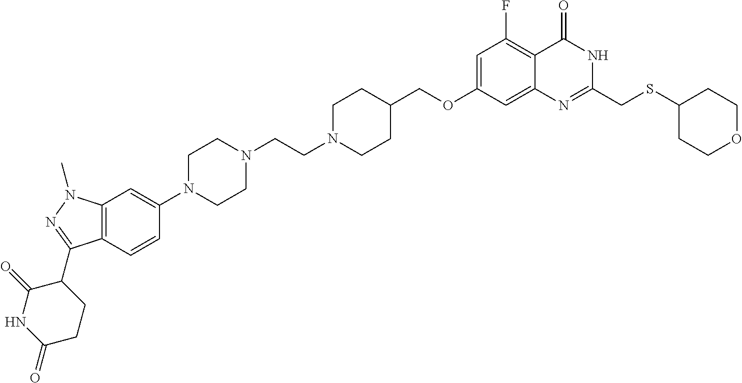 [SMILES: Cn1nc(C2CCC(=O)NC2=O)c2ccc(N3CCN(CCN4CCC(COc5cc(F)c6c(=O)[nH]c(CSC7CCOCC7)nc6c5)CC4)CC3)cc21] | LCMS (ESI, m/z): 761.35 [M + H]⁺; ¹H NMR (400 MHz, DMSO-d₆) δ 12.10 (s, 1H), 10.85 (s, 1H), 7.50 (d, J = 8.8 Hz, 1H), 7.95- 7.82 (m, 4H), 4.31-4.21 (m, 1H), 4.04- 3.97 (m, 2H), 3.95(s, 3H), 3.90- 3.79 (m, 2H), 3.64 (s, 2H), 3.48- |
| | 3-(6-(4-(2-(4-(((5-Fluoro-4-oxo-2-(((tetrahydro-2H-pyran- | 3.44 |
| | 4-yl)thio)methyl)-3,4-dihydroquinazolin-7- | (m, 2H), 3.26- |
| | yl)oxy)methyl)piperidin-1-yl)ethyl)piperazin-1-yl)-1- | 3.18 (m, |
| | methyl-1H-indazol-3-yl)piperidine-2,6-dione | 4H), 3.10- |
| | | 3.00(m, 1H), |
| | | 2.99-2.88 |
| | | (m, 2H), |
| | | 2.65-2.58 (m, |
| | | 7H), 2.33- |
| | | 2.28 |
| | | (m, 1H), |
| | | 2.20- |
| | | 1.70 (m, |
| | | 9H), 1.51- |
| | | 1.40 (m, 2H), |
| | | 1.38-1.23 (m, |
| | | 3H), 1.10- |
| | | 1.04 |
| | | (m, 1H). |
| 103 | 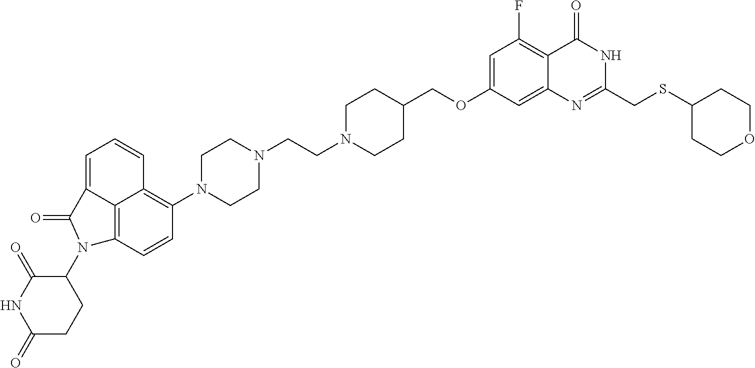 [SMILES: O=C1CCC(N2C(=O)c3cccc4c(N5CCN(CCN6CCC(COc7cc(F)c8c(=O)[nH]c(CSC9CCOCC9)nc8c7)CC6)CC5)ccc2c34)C(=O)N1] | LCMS (ESI, m/z): 798.35 [M + H]⁺; ¹H NMR (400 MHz, DMSO-d₆) δ 12.15 (br, 1H), 11.12 (s, 1H), 8.27 (d, J = 7.5 Hz, 1H), 8.10 (d, J = 6.9 Hz, 1H), 7.82 (t, J = 7.7 Hz, 1H), 7.00- 6.85 (m, 4H), 5.46- 5.32 (m, 1H), 4.01 (d, J = 5.7 Hz, |
| | 3-(6-(4-(2-(4-(((5-fluoro-4-oxo-2-(((tetrahydro-2H-pyran-4- | 2H), 3.85- |
| | yl)thio)methyl)-3,4-dihydroquinazolin-7- | 3.75 (M, |
| | yl)oxy)methyl)piperidin-1-yl)ethyl)piperazin-1-yl)-2- | 2H), 3.63 |
| | oxobenzo[cd]indol-1(2H)-yl)piperidine-2,6-dione | (s, 2H), |
| | | 3.40-3.30 |
| | | (m, 2H), |
| | | 3.18-3.01 |
| | | (m, 5H), |
| | | 3.00-2.85 |
| | | (m, 3H), |
| | | 2.80-2.50 |
| | | (m, 8H), |
| | | 2.10-1.85 |
| | | (m, 5H), |
| | | 1.82-1.70 |
| | | (m, 3H), |
| | | 1.55-1.20 |
| | | (m, 6H). |
| 104 | 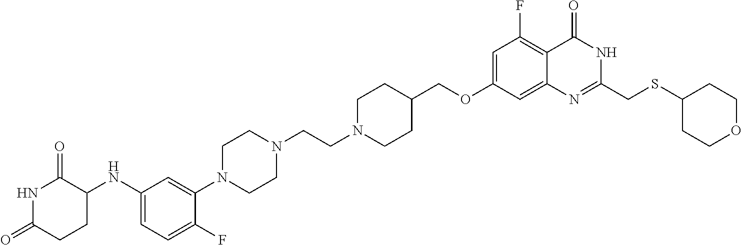 [SMILES: O=C1CCC(Nc2ccc(F)c(N3CCN(CCN4CCC(COc5cc(F)c6c(=O)[nH]c(CSC7CCOCC7)nc6c5)CC4)CC3)c2)C(=O)N1] | LCMS (ESI, m/z): 740.35 [M + H]⁺; ¹H NMR (300 MHz, DMSO-d₆) δ 12.10 (br, 1H), 10.74 (s, 1H), 7.03- 6.71 (m, 3H), 6.35 (dd, J = 7.8, 2.3 Hz, 1H), |
| | 3-((4-fluoro-3-(4-(2-(4-(((5-fluoro-4-oxo-2-(((tetrahydro- | 6.31-6.14 (m, |
| | 2H-pyran-4-yl)thio)methyl)-3,4-dihydroquinazolin-7- | 1H), 5.67 (d, |
| | yl)oxy)methyl)piperidin-1-yl)ethyl)piperazin-1- | J = 7.5 Hz, |
| | yl)phenyl)amino)piperidine-2,6-dione | 1H), 4.35- |
| | | 4.19 (m, |
| | | 1H), 3.98 |
| | | (d, J = 5.9 |
| | | Hz, 2H), |
| | | 3.92-3.72 (m, |
| | | 2H), 3.62 |
| | | (s, 2H), |
| | | 3.41-3.32 (m, |
| | | 3H), 3.12- |
| | | 3.01 |
| | | (m, 1H), 3.01- |
| | | 2.83 (m, |
| | | 6H), 2.82- |
| | | 2.65 (m, 2H), |
| | | 2.64-2.52 (m, |
| | | 4H), 2.50- |
| | | 2.40(m, |
| | | 3H), 2.17- |
| | | 2.05 |
| | | (m, 1H), 2.05- |
| | | 1.79 (m, |
| | | 5H), 1.80- |
| | | 1.63 (m, 3H), |
| | | 1.54-1.41 (m, |
| | | 2H), 1.36-1.21 |
| | | (m, 2H) |
| 105 | 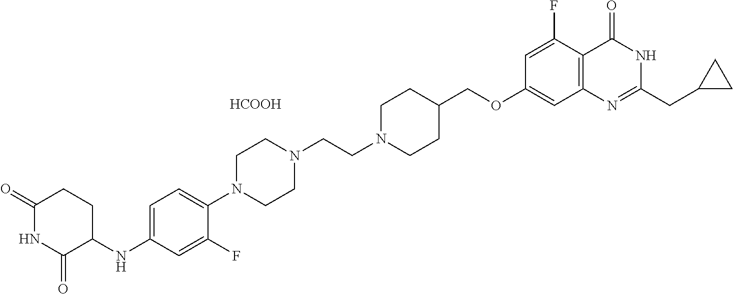 [SMILES: O=C1CCC(Nc2ccc(N3CCN(CCN4CCC(COc5cc(F)c6c(=O)[nH]c(CC7CC7)nc6c5)CC4)CC3)c(F)c2)C(=O)N1 ; "HCOOH"] | LCMS (ESI, m/z): 644.35 [M + H]⁺; 1H NMR (400 MHz, DMSO-d6) δ 12.03 (s, 1H), 10.77 (s, 1H), 8.17 (s, 1H), 6.98-6.74 (m, 3H), 6.51 (dd, J = 15.1, 2.6 Hz, 1H), 6.42 (dd, J = 8.6, |
| | 3-((4-(4-(2-(4-(((2-(cyclopropylmethyl)-5-fluoro-4-oxo-3,4- | 2.6 Hz, |
| | dihydroquinazolin-7-yl)oxy)methyl)piperidin-1- | 1H), 5.80 |
| | yl)ethyl)piperazin-1-yl)-3-fluorophenyl)amino)piperidine- | (d, J = 7.7 |
| | 2,6-dione formate | Hz, 1H), |
| | | 4.31-4.21 (m, |
| | | 1H), 3.98 (d, |
| | | J = 5.9 Hz, |
| | | 2H), 3.23- |
| | | 3.14 (m, |
| | | 2H), 2.96 |
| | | (d, J = |
| | | 11.1 Hz, |
| | | 2H), 2.89- |
| | | 2.80 |
| | | (m, 3H), |
| | | 2.79- |
| | | 2.70 (m, |
| | | 1H), 2.63- |
| | | 2.57 (m, 1H), |
| | | 2.53-2.40 |
| | | (m, 9H), |
| | | 2.14-1.97 (m, |
| | | 3H), 1.94- |
| | | 1.67 |
| | | (m, 4H), 1.40- |
| | | 1.22 (m, |
| | | 2H), 1.22- |
| | | 1.09 (m, 1H), |
| | | 0.53-0.44 (m, |
| | | 2H), 0.31- |
| | | 0.23 |
| | | (m, 2H). |
| 106 | 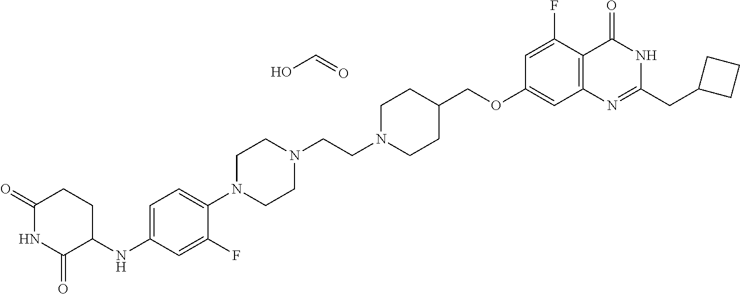 [SMILES: O=C1CCC(Nc2ccc(N3CCN(CCN4CCC(COc5cc(F)c6c(=O)[nH]c(CC7CCC7)nc6c5)CC4)CC3)c(F)c2)C(=O)N1 ; O=CO] | LCMS (ESI, m/z): 678.30 [M + H]⁺; 1H NMR (300 MHz, DMSO-d6) δ 11.98 (s, 1H), 10.76 (s, 1H), 8.17 (s, 1H), 6.93-6.82 (m, 2H), 6.80 (d, J = 2.7 Hz, 1H), 6.51 (dd, J = 15.0, 2.6 Hz, 1H), |
| | 3-((4-(4-(2-(4-(((2-(cyclobutylmethyl)-5-fluoro-4-oxo-3,4- | 6.42 (dd, |
| | dihydroquinazolin-7-yl)oxy)methyl)piperidin-1- | J = 8.9, 2.6 |
| | yl)ethyl)piperazin-1-yl)-3-fluorophenyl)amino)piperidine- | Hz, 1H), |
| | 2,6-dione formate | 5.78 (d, |
| | | J = 7.6 Hz, |
| | | 1H), 4.31- |
| | | 4.19 |
| | | (m, 1H), |
| | | 3.98 (d, J = |
| | | 5.9 Hz, |
| | | 2H), 3.72- |
| | | 3.48 (m, 5H), |
| | | 2.96 (d, |
| | | J = 10.8 |
| | | Hz, 3H), |
| | | 2.91- |
| | | 2.80 (m, |
| | | 4H), 2.80- |
| | | 2.68 (m, 3H), |
| | | 2.67-2.56 (m, |
| | | 4H), 2.13- |
| | | 1.95 |
| | | (m, 5H), 1.91- |
| | | 1.68 (m, |
| | | 8H), 1.40- |
| | | 1.27 (m, 2H). |
| 107 | 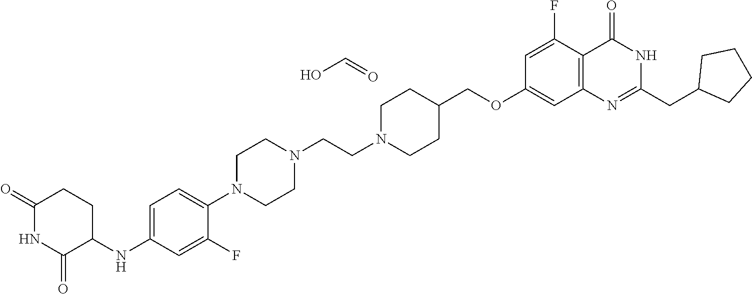 | LCMS (ESI, m/z): 692.35 [M + H]⁺; 1H NMR (400 MHz, DMSO-d6) δ 12.01 (br, 1H), 10.76 (s, 1H), 8.21 (s, 1H), 6.88-6.80 (m, 3H), 6.51 (dd, J = 15.0, 2. Hz, 1H), 6.42 (dd, J = 8.7, 2.6 Hz, |
| | 3-((4-(4-(2-(4-(((2-(cyclopentylmethyl)-5-fluoro-4-oxo-3,4- | 1H), 5.79 |
| | dihydroquinazolin-7-yl)oxy)methyl)piperidin-1- | (d, J = 7.6 |
| | yl)ethyl)piperazin-1-yl)-3-fluorophenyl)amino)piperidine- | Hz, 1H), |
| | 2,6-dione formate | 4.29-4.22 (m, |
| | | 1H), 3.98 (d, |
| | | J = 5.9 Hz, |
| | | 2H), 3.01 |
| | | (d, J = |
| | | 10.7 Hz, |
| | | 2H), 2.85 |
| | | (t, J = 4.6 |
| | | Hz, 3H), |
| | | 2.76- |
| | | 2.70 (m, |
| | | 1H), 2.56- |
| | | 2.52 (m, 7H), |
| | | 2.47-2.23 (m, |
| | | 1H), 2.16- |
| | | 2.06 |
| | | (m, 3H), |
| | | 1.82- |
| | | 1.43 (m, |
| | | 9H), 1.43- |
| | | 1.32 (m, 2H), |
| | | 1.31-1.08 (m, |
| | | 2H). |
| 108 | 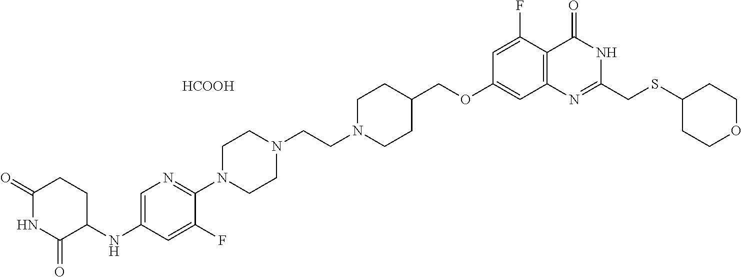 [SMILES: O=C1CCC(Nc2cnc(N3CCN(CCN4CCC(COc5cc(F)c6c(=O)[nH]c(CSC7CCOCC7)nc6c5)CC4)CC3)c(F)c2)C(=O)N1 ; "HCOOH"] | LCMS (ESI, m/z): 741.35 [M + H]⁺; 1H NMR (400 MHz, DMSO-d6) δ 12.16 (br, 1H), 10.80 (s, 1H), 8.19 (s, 1H), 7.57 (d, J = 8.2 Hz, 1H), 7.00 (dd, J = 14.8, 2.4 Hz, 1H), 6.96-6.83 (m, |
| | 3-((5-fluoro-6-(4-(2-(4-(((5-fluoro-4-oxo-2-(((tetrahydro- | 2H), 5.92 (d, |
| | 2H-pyran-4-yl)thio)methyl)-3,4-dihydroquinazolin-7- | J = 7.8 Hz, |
| | yl)oxy)methyl)piperidin-1-yl)ethyl)piperazin-1-yl)pyridin- | 1H), 4.34- |
| | 3-yl)amino)piperidine-2,6-dione | 4.25 (m, |
| | | 1H), 3.98 |
| | | (d, J = 5.9 |
| | | Hz, 2H), |
| | | 3.86-3.78 |
| | | (m, 2H), |
| | | 3.62 (s, |
| | | 2H), 3.39- |
| | | 3.35 (m, |
| | | 2H), 3.32- |
| | | 3.27 (m, |
| | | 2H), 3.14- |
| | | 3.02 (m, |
| | | 6H), 2.96 |
| | | (d, J = |
| | | 10.9 Hz, |
| | | 3H), 2.79- |
| | | 2.72 |
| | | (m, 1H), |
| | | 2.70- |
| | | 2.55 (m, |
| | | 5H), 2.13- |
| | | 2.07 (m, 1H), |
| | | 2.06-1.99 |
| | | (m 2H), |
| | | 1.94-1.83 (m, |
| | | 3H), 1.78- |
| | | 1.70 (m, |
| | | 3H), 1.52- |
| | | 1.40 (m, |
| | | 2H), 1.37- |
| | | 1.27 |
| | | (m, 2H). |

### Example 99: Synthesis of 3-((3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1l-yl)ethyl)piperazin-1-yl)phenyl)amino)piperidine-2,6-dione

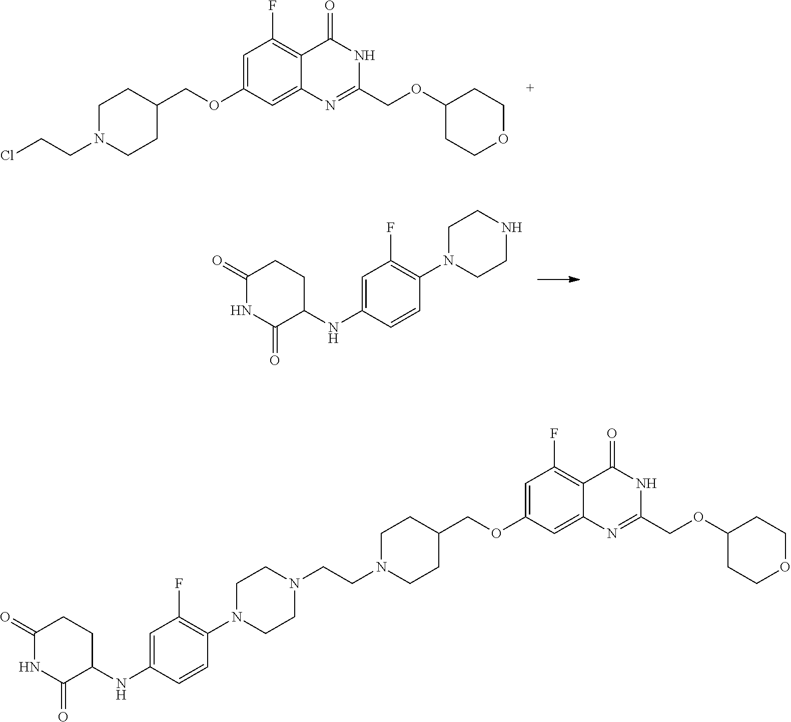 [SMILES: O=c1[nH]c(COC2CCOCC2)nc2cc(OCC3CCN(CCCl)CC3)cc(F)c12 ; O=C1CCC(Nc2ccc(N3CCNCC3)c(F)c2)C(=O)N1 ; O=C1CCC(Nc2ccc(N3CCN(CCN4CCC(COc5cc(F)c6c(=O)[nH]c(COC7CCOCC7)nc6c5)CC4)CC3)c(F)c2)C(=O)N1]

A solution of 7-((1-(2-chloroethyl)piperidin-4-yl)methoxy)-5-fluoro-2-(((tetrahydro-2H-pyran-4-yl)oxy)methyl)quinazolin-4(3H)-one (522 mg, 1.15 mmol, 1 equiv), 3-((3-fluoro-4-(piperazin-1-yl)phenyl)amino)piperidine-2,6-dione (423 mg, 1.38 mmol, 1.2 equiv) and DIEA (446 mg, 3.45 mmol, 3 equiv) in DMSO (5 mL) was stirred for 2 hours at 80° C. After concentration, the residue was purified by C18 reverse phase chromatography eluting with water/ACN (14:86) and further purified by Prep-HPLC with the following conditions (Column: XBridge Prep Phenyl Column, 19*250 mm, 5 μm; Mobile Phase A: Water (10 mmol/L NH₄HCO₃), Mobile Phase B: ACN; Flow rate: 25 mL/min; Gradient: 30% B to 40% B in 9 min; Wavelength: 254 nm/220 nm; RT (min): 9) to afford 3-((3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione (159 mg, 19%) as a white solid. LCMS (ESI, m/z): 724.20 [M+H]⁺. 1H NMR (400 MHz, DMSO-d₆) δ 12.01 (s, 1H), 10.77 (s, 1H), 6.95-6.87 (m, 2H), 6.85-6.78 (m, 1H), 6.65 (d, J=15.1, 2.6 Hz, 1H), 6.42 (d, J=8.5, 2.6 Hz, 1H), 5.80 (d, J=7.6 Hz, 1H), 4.38 (s, 2H), 4.31-4.21 (m, 1H), 3.98 (d, J=6.0 Hz, 2H), 3.87-3.75 (m, 2H), 3.70-3.60 (m, 1H), 3.39-3.35 (m, 3H), 2.97-2.80 (m, 6H), 2.80-2.71 (m, 2H), 2.60-2.58 (m, 1H), 2.49-2.38 (m, 4H), 2.13-2.06 (m, 1H), 2.01-1.68 (m, 9H), 1.59-1.41 (m, 2H), 1.35-1.19 (m, 3H).

Examples 99a and 99b: Separation of (S)-3-((3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione and (R)-3-((3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione

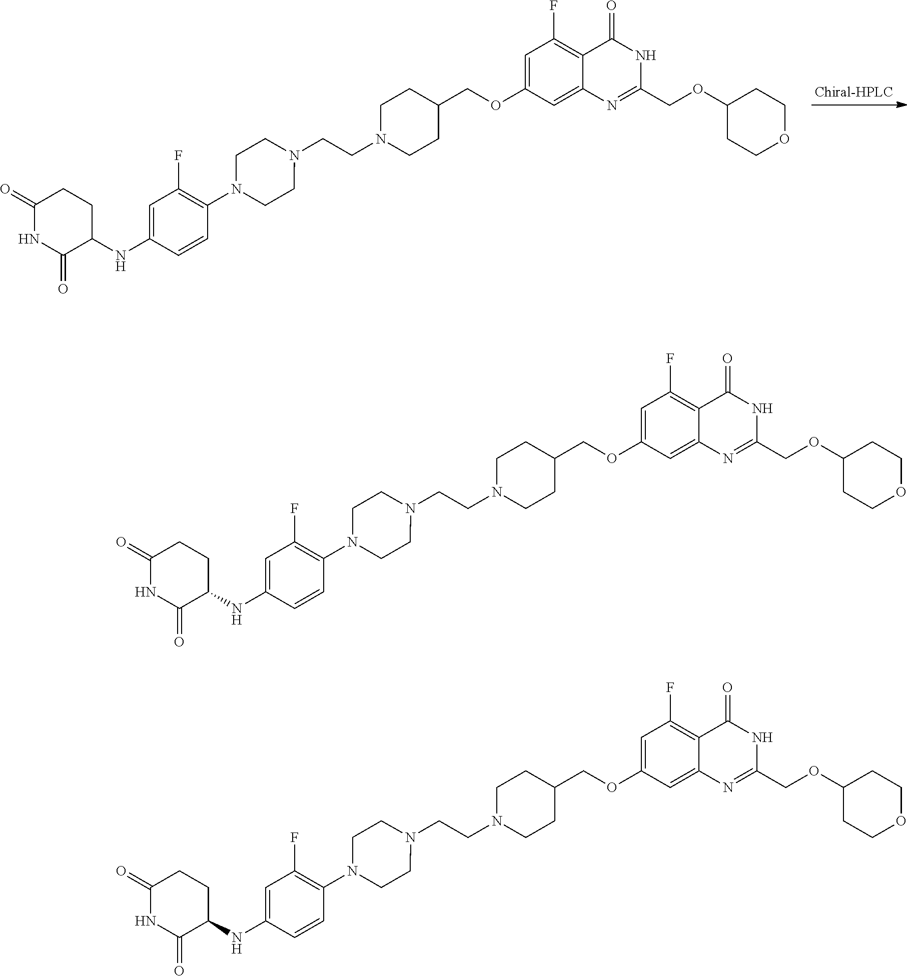 [SMILES: O=C1CCC(Nc2ccc(N3CCN(CCN4CCC(COc5cc(F)c6c(=O)[nH]c(COC7CCOCC7)nc6c5)CC4)CC3)c(F)c2)C(=O)N1 ; O=C1CC[C@H](Nc2ccc(N3CCN(CCN4CCC(COc5cc(F)c6c(=O)[nH]c(COC7CCOCC7)nc6c5)CC4)CC3)c(F)c2)C(=O)N1 ; O=C1CC[C@@H](Nc2ccc(N3CCN(CCN4CCC(COc5cc(F)c6c(=O)[nH]c(COC7CCOCC7)nc6c5)CC4)CC3)c(F)c2)C(=O)N1]

500 mg of the racemic 3-((3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione (Example 99) was separated by Chiral-Prep-HPLC with the following conditions: Column: CHIRALPAK IF, 2*25 cm, 5 μm; Mobile Phase A: Hexane (0.5% 2M NH₃-MeOH), Mobile Phase B: EtOH/DCM=1:1; Flow rate: 20 mL/min; Isocratic: 80% B; Wavelength: 220 nm/254 nm; RT (min): 9.27 (Example 99a); RT (min): 22.70 (Example 99b); Sample Solvent: EtOH/DCM=1:1-HPLC; Injection Volume: 1.0 mL; Number of runs: 6). The fractions were directly concentrated and then lyophilized to afford the title compounds. Absolute stereochemistry was not assigned.

Example 99a: The first eluting isomer isolated using the above conditions (217 mg) as a white solid. LCMS (ESI, m/z): 724.30 [M+H]⁺. 1H NMR (400 MHz, DMSO-d6) δ 12.04 (s, 1H), 10.77 (s, 1H), 6.98-6.80 (m, 3H), 6.54 (d, J=2.0 Hz, 1H), 6.47-6.37 (m, 1H), 5.82 (d, J=7.7 Hz, 1H), 4.38 (s, 2H), 4.31-4.20 (m, 1H), 4.04 (d, J=6.2 Hz, 2H), 3.88-3.78 (m, 2H), 3.70-3.63 (m, 2H), 3.39-3.30 (m, 4H), 2.95-2.83 (m, 4H), 2.81-2.54 (m, 9H), 2.15-1.68 (m, 8H), 1.60-1.40 (m, 4H), 1.29-1.21 (i, 1H). Chiral PLC: CHIRALPAK IF-3; Mobile Phase A: Hex (0.2% M DEA): (EtOH:DCM=1:1)=20 80, Flow rate: 1.0 mL/min; Retention time: 2.13 mi (faster peak).

Example 99b: The second eluting isomer isolated using the above conditions (173 mg) as a white solid. LCMS (ESI, m/z): 724.30 [M+H]⁺. 1H NMR (400 MHz, DMSO-d₆) δ 12.03 (s, 1H), 10.77 (s, 1H), 7.00-6.78 (m, 3H), 6.54 (d, J=2.0 Hz, 1H), 6.47-6.37 (m, 1H), 5.81 (d, J=7.6 Hz, 1H), 4.38 (s, 2H), 4.31-4.21 (m, 1H), 4.03 (d, J=6.1 Hz, 2H), 3.88-3.78 (m, 2H), 3.72-3.61 (m, 2H), 3.38-3.30 (m, 4H), 2.92-2.85 (m, 4H), 2.80-2.53 (m, 9H), 2.15-1.97 (m, 2H), 1.96-1.77 (m, 6H), 1.56-1.41 (m, 4H), 1.27-1.22 (m, 1H). Chiral HPLC: CHIRALPAK IF-3; Mobile Phase A: Hex (0.2% DEA) (EtOH DCM=1:1)=20:80; Flow rate: 1.0 mL/min; Retention time: 5.59 min (slower peak).

Examples 109-111 were synthesized according to the procedure described for the synthesis 3-((4-(3-((4-(((5-fluoro-2-((((1r,4r)-4-hydroxycyclohexyl)thio)methyl)-4-oxo-3,4-dihydroquinazolin-7-yl)oxy)methyl)piperidin-1-yl)methyl)cyclobutoxy)phenyl)amino)piperidine-2,6-dione formate (Example 90) using appropriate building blocks and modified reaction conditions (such as reagents, reagent ratio, temperature, and reaction time) and purification conditions as needed.

| Example | Structure | Analytical Data |
|---|---|---|
| 109 | 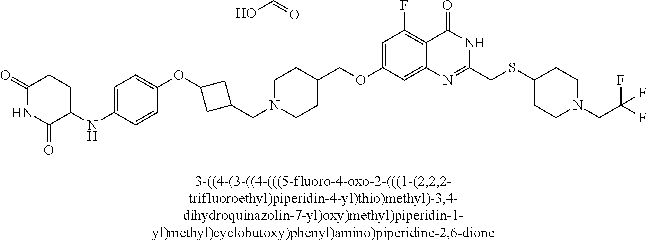 [SMILES: O=C1CCC(Nc2ccc(OC3CC(CN4CCC(COc5cc(F)c6c(=O)[nH]c(CSC7CCN(CC(F)(F)F)CC7)nc6c5)CC4)C3)cc2)C(=O)N1 ; O=CO] | LCMS (ESI, m/z): 775.15 [M + H]⁺; ¹H NMR (400 MHz, DMSO-d₆) δ 12.15 (s, 1H), 10.75 (s, 1H), 8.23 (s, 1H), 6.92- 6.85 (m, 2H), 6.64-6.58 (m, 4H), 5.41 (d, J = 7.3 Hz, 1H), 4.69- 4.60 (m, 1H), 4.22-4.15 (m, 1H), 3.98 (d, J = |
| | | 5.8 Hz, 2H), 3.60 |
| | | (s, 2H), 3.18- |
| | | 3.10 (m, 2H), |
| | | 2.90-2.81 (m, |
| | | 5H), 2.77-2.67 |
| | | (m, 2H), 2.62- |
| | | 2.58 (m, 1H), |
| | | 2.43-2.33 (m, |
| | | 4H), 2.15-2.09 |
| | | (m, 4H), 1.96- |
| | | 1.83 (m, 5H), |
| | | 1.78-1.70 (m, |
| | | 3H), 1.52-1.42 |
| | | (m, 2H), 1.32- |
| | | 1.23 (m, 3H). |
| 110 | 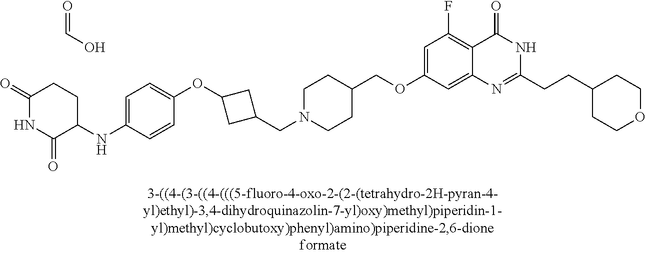 [SMILES: O=C1CCC(Nc2ccc(OC3CC(CN4CCC(COc5cc(F)c6c(=O)[nH]c(CCC7CCOCC7)nc6c5)CC4)C3)cc2)C(=O)N1 ; O=CO] | LCMS (ESI, m/z): 676.50 [M + H]⁺; ¹H NMR (400 MHz, DMSO-d₆) δ 12.05 (s, 1H), δ 10.75 (s, 1H), 8.19 (s, 1H), 6.87 (d, J = 2.5 Hz, 1H), 6.82 (dd, J = 12.5, 2.4 Hz, 1H), 6.61 (s, 4H), 5.45-5.39 (m, 1H), 4.64 (t, J = 6.3 Hz, 1H), 4.21- 4.13 (m, 1H), |
| | | 3.97 (d, J = 8.0 Hz |
| | | 2H), 3.83 (td, |
| | | J = 11.7, 2.1 Hz, |
| | | 2H), 3.29-3.20 (m, |
| | | 3H) 2.91 (d, J = |
| | | 10.9 Hz, 2H), |
| | | 2.78-2.66 (m, 1H), |
| | | 2.62-2.59 (m, |
| | | 3H), 2.19-2.10 |
| | | (m, 5H), 2.05- |
| | | 1.97 (m, 2H), |
| | | 1.90-1.80 (m, |
| | | 1H), 1.79-1.71 |
| | | (m, 2H), 1.68- |
| | | 1.57 (m, 5H), |
| | | 1.53-1.41 (m, |
| | | 1H), 1.38-1.29 |
| | | (m, 2H), 1.25- |
| | | 1.12 (m, 3H) |
| 111 | 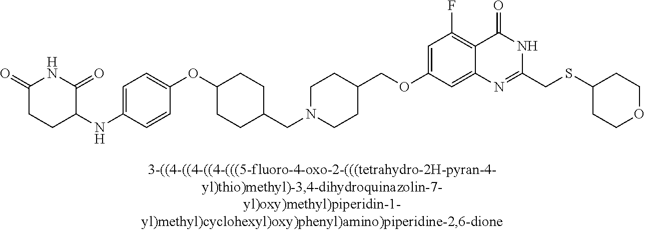 [SMILES: O=C1CCC(Nc2ccc(OC3CCC(CN4CCC(COc5cc(F)c6c(=O)[nH]c(CSC7CCOCC7)nc6c5)CC4)CC3)cc2)C(=O)N1] | LCMS (ESI, m/z): 722.90 [M + H]⁺; ¹H NMR (400 MHz, DMSO-d6) δ 12.15 (s, 1H), 10.76 (s, 1H), 8.20 (s, 1H) HCOOH, 6.92-6.84 (m, 2H), 6.72 (t, J = 8.2 Hz, 2H), 6.68- 6.58 (m, 2H), 5.58-5.44 (m, |
| | | 1H), 4.32 (s, 1H), |
| | | 4.25-4.15 (m, |
| | | 1H), 3.98 (d, J = |
| | | 5.9 Hz, 2H), 3.85- |
| | | 3.80 (m, 2H), |
| | | 3.62 (s, 2H), 3.38- |
| | | 3.27 (m, 3H), |
| | | 3.12-3.00 (m, |
| | | 2H), 2.98-2.85 |
| | | (m, 2H), 2.80- |
| | | 2.68 (m, 1H), |
| | | 2.61-2.55 (m, |
| | | 1H), 2.21 (d, J = |
| | | 7.0 Hz, 1H), 2.17- |
| | | 2.06 (m, 2H), |
| | | 2.05-1.73 (m, |
| | | 9H), 1.68-1.63 |
| | | (m, 1H), 1.55- |
| | | 1.22 (m, 9H). |

### Example 112: Synthesis of 3-((4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione

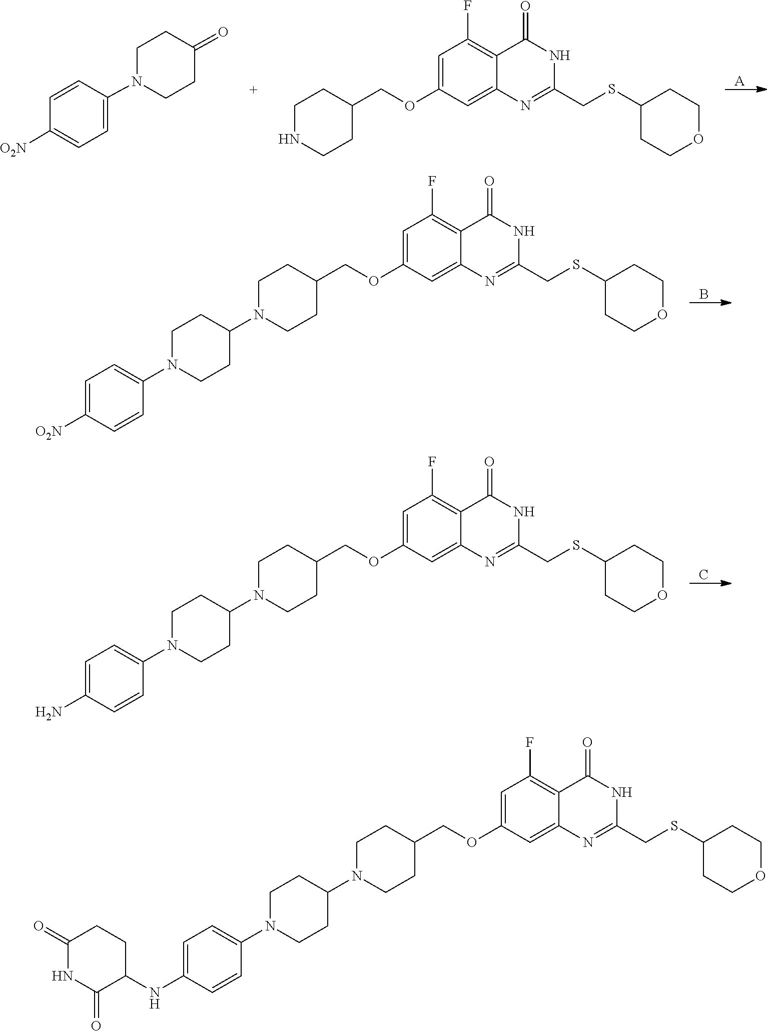

### Step A

A solution of 1-(4-nitrophenyl)piperidin-4-one (0.81 g, 3.68 mmol, 1.5 equiv) and 5-fluoro-2-[(oxan-4-ylsulfanyl)methyl]-7-(piperidin-4-ylmethoxy)-3H-quinazolin-4-one (1.0 g, 2.45 mmol, 1.00 equiv) and NaBH₃CN (0.46 g, 7.36 mmol, 3 equiv) in MeOH (10 mL) was stirred for 1 hour at 80° C. The resulting mixture was concentrated under vacuum. The residue was purified by silica gel column chromatography, eluting with DCM/MeOH (10:1) to afford 5-fluoro-7-((1′-(4-nitrophenyl)-[1,4′-bipiperidin]-4-yl)methoxy)-2-(((tetrahydro-2H-pyran-4-yl)thio)methyl)quinazolin-4(3H)-one (320 mg, 19%) as a yellow solid. LCMS (ESI, m/z): 612.25 [M+H]⁺.

### Step B

A solution of 5-fluoro-7-((1′-(4-nitrophenyl)-[1,4′-bipiperidin]-4-yl)methoxy)-2-(((tetrahydro-2H-pyran-4-yl)thio)methyl)quinazolin-4(3H)-one (150 mg, 0.245 mmol, 1 equiv), Fe (68.5 mg, 1.23 mmol, 5 equiv) and NH₄Cl (26.2 mg, 0.490 mmol, 2 equiv) in EtOH (10 mL) was stirred for 1 hour at 80° C. The resulting mixture was filtered, the filter cake was washed with EtOH (3×15 mL). The filtrate was concentrated under reduced pressure and the resulting residue was purified by silica gel column chromatography, eluting with DCM/MeOH (10:1) to afford 7-((1′-(4-aminophenyl)-[1,4′-bipiperidin]-4-yl)methoxy)-5-fluoro-2-(((tetrahydro-2H-pyran-4-yl)thio)methyl)quinazolin-4(3H)-one (104 mg, 60%) as a white solid. LCMS (ESI, m/z): 582.20 [M+H]⁺.

### Step C

A solution of 7-((1′-(4-aminophenyl)-[1,4′-bipiperidin]-4-yl)methoxy)-5-fluoro-2-(((tetrahydro-2H-pyran-4-yl)thio)methyl)quinazolin-4(3H)-one (90 mg, 0.155 mmol, 1 equiv) and 3-bromopiperidine-2,6-dione (59 mg, 0.31 mmol, 2 equiv) and DIEA (60 mg, 0.47 mmol, 3 equiv) in NMP (3 mL) was stirred for 1 hour at 90° C. The residue was purified by reverse-phase flash chromatography with the following conditions: column, C18 silica gel; mobile phase, MeCN in water (10 mmol/L NH₄HCO₃), 5% to 95% gradient in 25 min; detector, UV 254 nm to afford impure product that was further purified by Prep-HPLC with the following conditions (Column: Xselect CSH OBD Column 30*150 mm, 5 umn; Mobile Phase A: water (0.1% FA), Mobile Phase B: ACN; Flow rate: 60 mL/min; Gradient: 3% B to 18% B in 10 min; Wave Length: 254 nm/220 nm; RT (min): 8.5) to afford 3-((4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione (22 mg, 20%) as a dark grey solid. LCMS (ESI, m/z): 693.15 [M+H]⁺. ¹H NMR (400 MHz, DMSO-d₆) δ 12.16 (s, 1H), 10.74 (s, 1H), 8.19 (s, 1H), 6.93-6.84 (m, 2H), 6.76 (d, J=8.7 Hz, 2H), 6.60 (d, J=8.7 Hz, 2H), 5.36 (d, J=7.2 Hz, 1H), 4.23-4.14 (m, 1H), 3.98 (d, J=5.8 Hz, 2H), 3.86-3.76 (m, 2H), 3.62 (s, 2H), 3.56-3.52 (m, 2H), 3.31-3.30 (m, 2H), 3.11-3.01 (m, 1H), 3.00-2.91 (m, 2H), 2.79-2.66 (m, 1H), 2.63-2.58 (m, 1H), 2.58-2.54 (m, 1H), 2.47-2.44 (m, 1H), 2.41-2.31 (m, 1H), 2.21-2.14 (m, 2H), 2.15-2.06 (m, 1H), 1.93-1.71 (m, 8H), 1.65-1.52 (m, 2H), 1.52-1.37 (m, 2H), 1.37-1.22 (m, 2H).

Examples 113-117 were synthesized according to the procedure described for the synthesis 3-((4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione (Example 112) using appropriate building blocks and modified reaction conditions (such as reagents, reagent ratio, temperature, and reaction time) and purification conditions as needed.

| Example | Structure | Analytical Data |
|---|---|---|
| 113 | 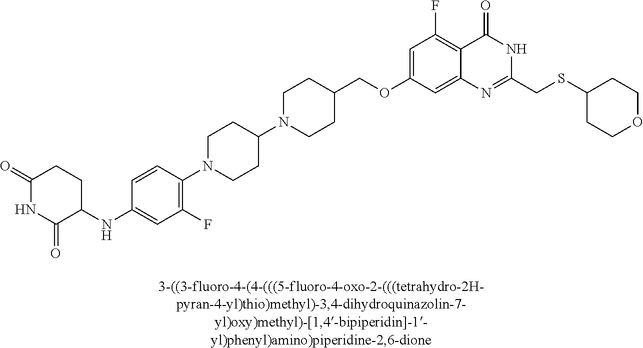 [SMILES: O=C1CCC(Nc2ccc(N3CCC(N4CCC(COc5cc(F)c6c(=O)[nH]c(CSC7CCOCC7)nc6c5)CC4)CC3)c(F)c2)C(=O)N1] | LCMS (ESI, m/z): 711.25 [M + H]⁺; ¹H NMR (300 MHz, DMSO-d₆) δ 12.10 (s, 1H), 10.76 (s, 1H), 6.90-6.75 (m, 3H), 6.61-6.28 (m, 2H), 5.76 (s, 1H), 4.30- 4.15 (m, 1H), 3.99 (s, 2H), 3.83 (d, J = 9.0 Hz, 3H), 3.63 (s, 3H), 3.30-3.05 (m, 4H), 2.92 (d, J = 12.0 Hz, 2H), 2.79-2.65 (m, 2H), 2.37-2.05 (m, 4H), 1.95-1.69 (m, 8H), 1.68-1.54 (m, 2H), 1.44-1.31 (m, 2H), 1.38-1.16 (m, 3H). |
| 114 | 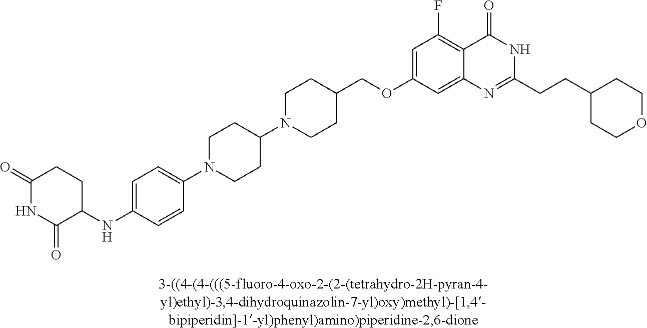 [SMILES: O=C1CCC(Nc2ccc(N3CCC(N4CCC(COc5cc(F)c6c(=O)[nH]c(CCC7CCOCC7)nc6c5)CC4)CC3)cc2)C(=O)N1] | LCMS (ESI, m/z): 675.45 [M + H]⁺; ¹H NMR (400 MHz, DMSO-d₆) δ 12.04 (s, 1H), 10.75 (s, 1H), 6.90-6.79 (m, 2H), 6.77 (d, J = 6.8 Hz, 2H), 6.63 (d, 9.2 Hz, 2H), 5.36 (d, J = 7.3 Hz, 1H), 4.37-4.10 (m, 1H), 3.97 (d, 5.6 Hz, 2H), 3.90- 3.77 (m, 2H), 3.46- 3.40 (m, 2H), 3.30- 3.22 (m, 3H), 3.02- 3.86 (m, 2H), 2.78- 2.66 (m, 1H), 2.60- 2.55 (m, 2H), 2.48- 2.44 (m, 1H), 2.36- |
| | | 2.26 (m, 1H), 2.23- |
| | | 2.05 (m, 3H), 1.90- |
| | | 1.70 (m, 6H), 1.70- |
| | | 1.40 (m, 7H), 1.35- |
| | | 1.10 (m, 5H). |
| 115 | 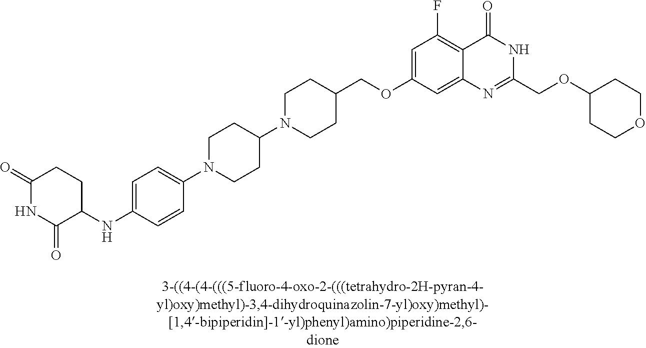 [SMILES: O=C1CCC(Nc2ccc(N3CCC(N4CCC(COc5cc(F)c6c(=O)[nH]c(COC7CCOCC7)nc6c5)CC4)CC3)cc2)C(=O)N1] | LCMS (ESI, m/z): 677.55 [M + H]⁺; ¹H NMR (400 MHz, DMSO-d₆) δ 12.08 (s, 1H), 10.79 (s, 1H), 9.39 (br, 1H), 7.10-6.84 (m, 4H), 6.69 (d, J = 8.3 Hz, 2H), 4.38 (s, 2H), 4.30-4.20 (m, 1H), 4.06 (d, J = 6.0 Hz, 2H), 3.88-3.64 (m, 6H), 3.39-3.26 (m, 5H), , 3.07 (d, J = 12.0 Hz, 2H), 2.80- 2.65 (m, 2H), 2.61 (d, J = 4.6 Hz, 1H), 2.37- 1.99 (m, 6H), 1.97- 1.77 (m, 5H), 1.68- 1.55 (m, 2H), 1.54- 1.41 (m, 2H). |
| 116 | 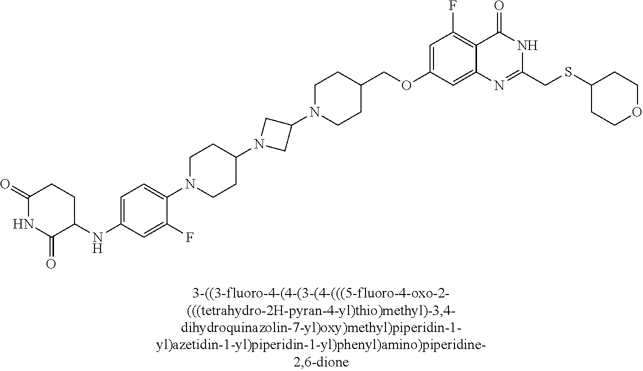 [SMILES: O=C1CCC(Nc2ccc(N3CCC(N4CC(N5CCC(COc6cc(F)c7c(=O)[nH]c(CSC8CCOCC8)nc7c6)CC5)C4)CC3)c(F)c2)C(=O)N1] | LCMS (ESI, m/z): 766.25 [M + H]⁺; ¹H NMR (300 MHz, DMSO-d₆) δ 12.12 (s, 1H), 10.75 (s, 1H), 6.98-6.71 (m, 3H), 6.54-6.36 (m, 2H), 5.76 (d, J = 7.6 Hz, 1H), 4.30-4.20 (m, 1H), 3.98 (d, J = 5.7 Hz, 2H), 3.89- 3.73 (m, 2H), 3.62 (s, 2H), 3.41-3.30 (m, 4H), 3.13-2.96 (m, 3H), 2.76 (m, 6H), 2.59 (d, J = 9.0 Hz, 3H), 2.14-2.01 (m, 2H), 1.89-1.65 (m, 10H), 1.53-1.40 (m, 2H), 1.38-1.16 (m, 4H). |
| 117 | 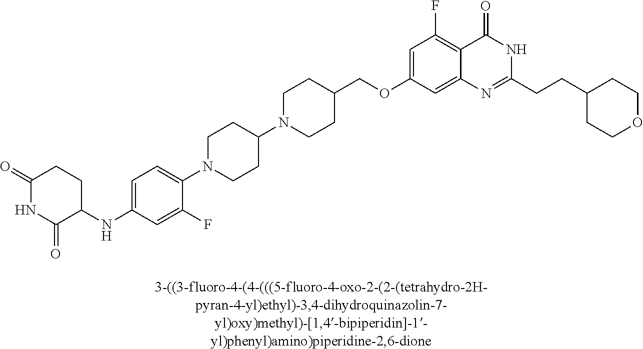 [SMILES: O=C1CCC(Nc2ccc(N3CCC(N4CCC(COc5cc(F)c6c(=O)[nH]c(CCC7CCOCC7)nc6c5)CC4)CC3)c(F)c2)C(=O)N1] | LCMS (ESI, m/z): 693.40 [M+H]+; 1H NMR (400 MHz, DMSO-d6) & 12.08 (s, 1H), 10.77 (s, 1H), 9.14 (br, 1H), 6.92-6.82 (m, 3H), 6.52 (dd, J = 12.0 Hz, 4.0 Hz, 1H), 6.43 (d, J = 12.0 Hz, 4.0 Hz, 1H), 5.87 (d, J = 7.6 Hz, 1H), 4.38-4.15 (m, 1H), 4.05 (d, J = 4.3 Hz, 2H), 3.84 (dd, J = 11.0, 4.3 Hz, 2H), 3.56 (d, J = 7.2 Hz, 1H), 3.31-3.10 (m, 5H), 3.11-2.99 (m, 1H), 2.82-2.55 (m, 6H), 2.40-1.91 (m, 6H), 2.05-1.78 |
| | | (m, 4H), 1.75-1.55 |
| | | (m, 6H), 1.55-1.42 |
| | | (m, 1H), 1.29-1.03 |
| | | (m, 2H). |

### Example 116: Synthesis of 3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione

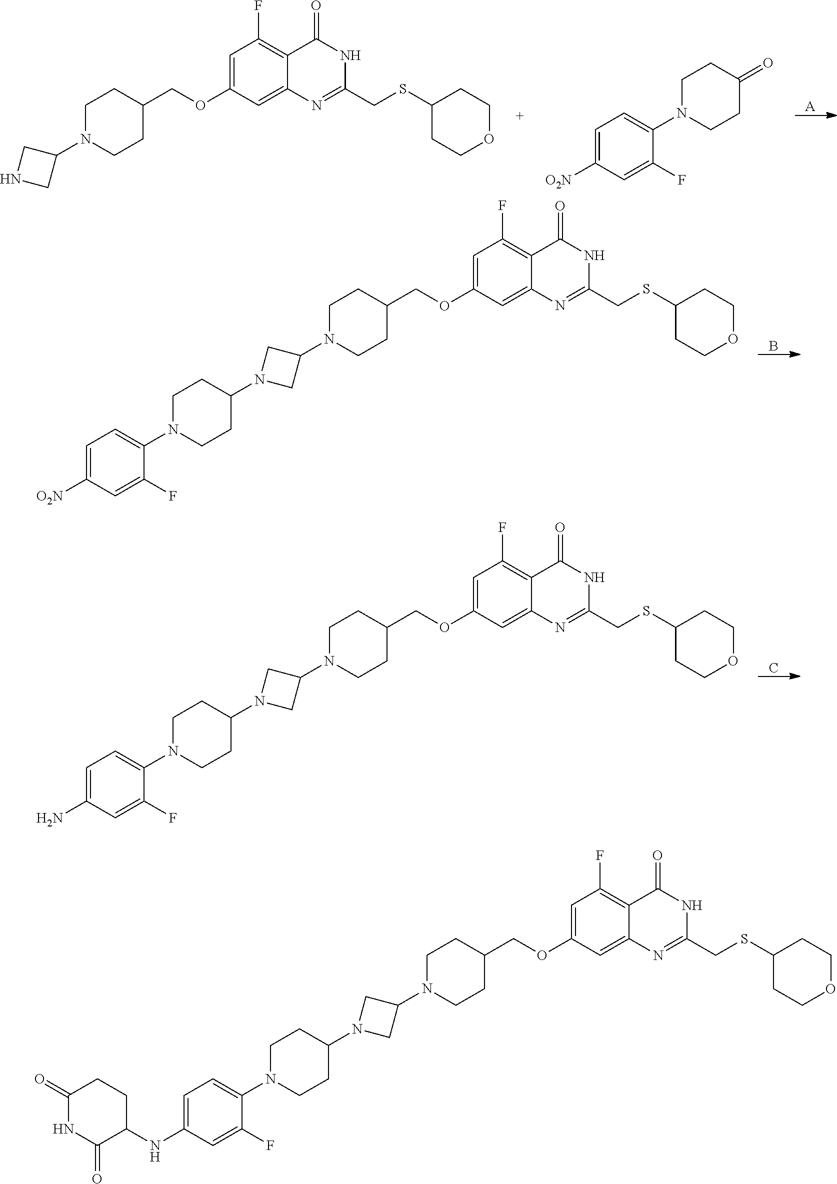

### Step A

A solution of 1-(2-fluoro-4-nitrophenyl)piperidin-4-one (1.2 g, 5.0 mmol, 1 equiv), 7-((1-(azetidin-3-yl)piperidin-4-yl)methoxy)-5-fluoro-2-(((tetrahydro-2H-pyran-4-yl)thio)methyl)quinazolin-4(3H)-one (2.33 g, 5.04 mmol, 1 equiv) and STAB (3.20 g, 15.1 mmol, 3 equiv) in DCM (50 mL) was stirred overnight. The solution was concentrated and the residue was purified by silica gel column chromatography, eluting with CH₂Cl₂/MeOH (9:1) to afford 5-fluoro-7-((1-(1-(1-(2-fluoro-4-nitrophenyl)piperidin-4-yl)azetidin-3-yl)piperidin-4-yl)methoxy)-2-(((tetrahydro-2H-pyran-4-yl)thio)methyl)quinazolin-4(3H)-one (1.4 g, 41%) as a light-yellow oil. LCMS (ESI, m/z): 685.40 [M+H]⁺.

### Step B

A solution of 5-fluoro-7-((1-(1-(1-(2-fluoro-4-nitrophenyl)piperidin-4-yl)azetidin-3-yl)piperidin-4-yl)methoxy)-2-(((tetrahydro-2H-pyran-4-yl)thio)methyl)quinazolin-4(3H)-one (1.0 g, 1.5 mmol, 1 equiv) in DMF (20 mL) was treated with B₂(OH)₄ (0.39 g, 4.38 mmol, 3 equiv) for 2 min then bipyridine (0.02 g, 0.146 mmol, 0.1 equiv) was added portion-wise and stirred for 10 min.

The mixture was concentrated, and the residue was purified by reversed-phase flash chromatography with the following conditions: column, C18 silica gel; mobile phase, ACN in water (10 mmol/L NH₄HCO₃), 0% to 70% gradient in 30 min; detector, UV 254 nm. This resulted in 7-((1-(1-(1-(4-amino-2-fluorophenyl)piperidin-4-yl)azetidin-3-yl)piperidin-4-yl)methoxy)-5-fluoro-2-(((tetrahydro-2H-pyran-4-yl)thio)methyl)quinazolin-4(3H)-one (549 mg, 57%) as a grey solid. LCMS (ESI, m/z): 655.45 [M+H]⁺.

### Step C

A solution of 7-((1-(1-(1-(4-amino-2-fluorophenyl)piperidin-4-yl)azetidin-3-yl)piperidin-4-yl)methoxy)-5-fluoro-2-(((tetrahydro-2H-pyran-4-yl)thio)methyl)quinazolin-4(3H)-one (740 mg, 1.13 mmol, 1 equiv), 3-bromopiperidine-2,6-dione (651 mg, 3.39 mmol, 3 equiv) and NaHCO₃ (475 mg, 5.65 mmol, 5 equiv) in ACN (30 mL) was stirred for 2 days at 90° C. The mixture was concentrated and the residue was purified by reverse-phase flash chromatography with the following conditions: column, C18 silica gel; mobile phase, MeCN in water (0.1% FA), 0% to 70% gradient in 30 min; detector, UV 254 nm. The resulting product was purified by Prep-HPLC with the following conditions (Column: YMC-Actus Triart C18 20*250 mm, 5 μm, 12 nm; Mobile Phase A: Water (10 mmol/L NH₄HCO₃), Mobile Phase B: ACN; Flow rate: 20 mL/min; Gradient: 35%-45% B; Wavelength: 254 nm; RT (min): 8) to afford 3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione (144 mg, 17%) as a white solid. LCMS (ESI, m/z): 766.25 [M+H]⁺. 1H NMR (300 MHz, DMSO-d₆) δ 12.12 (br s, 1H), 10.75 (s, 1H), 6.98-6.71 (m, 3H), 6.54-6.36 (m, 2H), 5.76 (d, J=7.6 Hz, 1H), 4.30-4.20 (m, 1H), 3.98 (d, J=5.7 Hz, 2H), 3.89-3.73 (m, 2H), 3.62 (s, 2H), 3.41-3.30 (m, 4H), 3.13-2.96 (m, 3H), 2.76 (dd, J=17.0, 7.4 Hz, 6H), 2.59 (d, J=9.0 Hz, 3H), 2.14-2.01 (m, 2H), 1.89-1.65 (m, 10H), 1.53-1.40 (m, 2H), 1.38-1.16 (m, 4H).

Examples 116a and 116b: Separation of (S)-3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione and (R)-3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione.

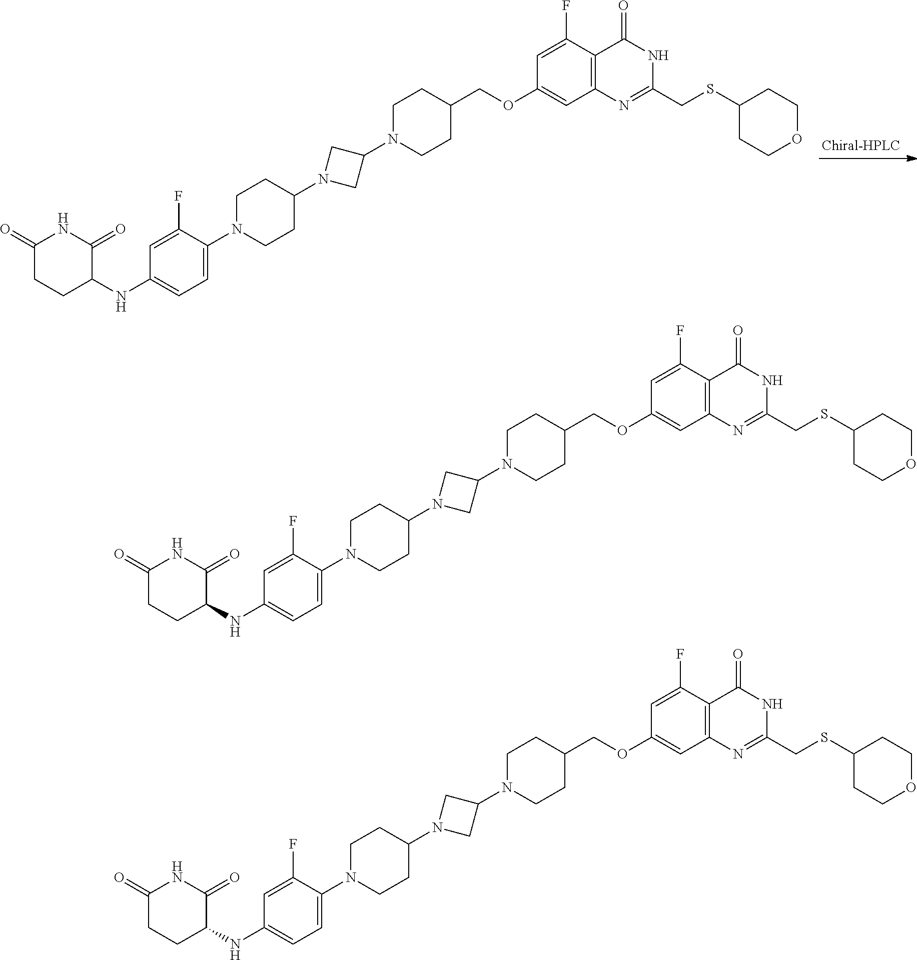 [SMILES: O=C1CCC(Nc2ccc(N3CCC(N4CC(N5CCC(COc6cc(F)c7c(=O)[nH]c(CSC8CCOCC8)nc7c6)CC5)C4)CC3)c(F)c2)C(=O)N1 ; O=C1CC[C@H](Nc2ccc(N3CCC(N4CC(N5CCC(COc6cc(F)c7c(=O)[nH]c(CSC8CCOCC8)nc7c6)CC5)C4)CC3)c(F)c2)C(=O)N1 ; O=C1CC[C@@H](Nc2ccc(N3CCC(N4CC(N5CCC(COc6cc(F)c7c(=O)[nH]c(CSC8CCOCC8)nc7c6)CC5)C4)CC3)c(F)c2)C(=O)N1]

300 mg of the racemic 3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione (Example 116) was separated by Chiral-Prep-HPLC with the following conditions: (Column: CHIRALPAK IF, 2*25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: isocratic 80% B; Wavelength: 220 nm/254 nm; RT (min): 7.10 (Example 116a); RT (min): 12.17 (Example 116b); Sample Solvent: EtOH:DCM=1:1-HPLC; Injection Volume: 1.0 mL; Number of runs: 5). The fractions were concentrated and then lyophilized to afford the title compounds. Absolute stereochemistry was not assigned.

Example 116a: The first eluting isomer isolated using the above conditions (88 mg) as a white solid. LCMS (ESI, m/z): 766.35 [M+H]⁺. 1H NMR (300 MHz, DMSO-d6) δ 12.13 (br, 1H), 10.76 (s, 1H), 6.95-6.80 (m, 3H), 6.50 (dd, J=14.9, 2.5 Hz, 1H), 6.41 (dd, J=8.5, 2.6 Hz, 1H), 5.77 (d, J=7.5 Hz, 1H), 4.31-4.18 (m, 1H), 3.98 (d, J=5.7 Hz, 2H), 3.85-3.76 (m, 2H), 3.63 (s, 2H), 3.44-3.27 (m, 4H), 3.13-2.99 (m, 3H), 2.91-2.67 (m, 6H), 2.63-2.55 (m, 3H), 2.17-2.03 (m, 2H), 1.95-1.64 (m, 10H), 1.55-1.36 (m, 2H), 1.34-1.21 (m, 4H). Chiral HPLC: CHIRALPAK IF-3; Mobile Phase A: Hex (0.2% DEA):(EtOH:DCM=1:1)=20:80; Flow rate: 1.0 mL/min; Retention time: 1.52 min (faster peak).

Example 116b: The second eluting isomer isolated using the above conditions (90 mg) as a white solid. LCMS (ESI, m/z): 766.35 [M+H]⁺. 1H NMR (300 MHz, DMSO-d6) δ 12.14 (br, 1H), 10.76 (s, 1H), 6.94-6.77 (m, 3H), 6.50 (dd, J=14.8, 2.5 Hz, 1H), 6.41 (d, J=9.0 Hz, 1H), 5.77 (d, J=7.6 Hz, 1H), 4.32-4.18 (m, 1H), 3.98 (d, J=5.7 Hz, 2H), 3.82-3.75 (m, 2H), 3.63 (s, 2H), 3.47-3.29 (m, 4H), 3.13-3.03 (m, 3H), 2.84-2.65 (m, 6H), 2.64-2.55 (m, 3H), 2.10 (d, J=10.1 Hz, 2H), 1.95-1.64 (m, 10H), 1.55-1.38 (m, 2H), 1.34-1.21 (m, 4H). Chiral HPLC: CHIRALPAK IF-3; Mobile Phase A: Hex (0.2% DEA):(EtOH:DCM=1:1)=20:80; Flow rate: 1.0 mL/min; Retention time: 2.53 min (slower peak).

Examples 117a and 117b: Separation of (S)-3-((3-fluoro-4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidin]-1′-yl)phenyl)amino)piperidine-2,6-dione and (R)-3-((3-fluoro-4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidin]-1′-ylphenyl amino)piperidine-2,6-dione

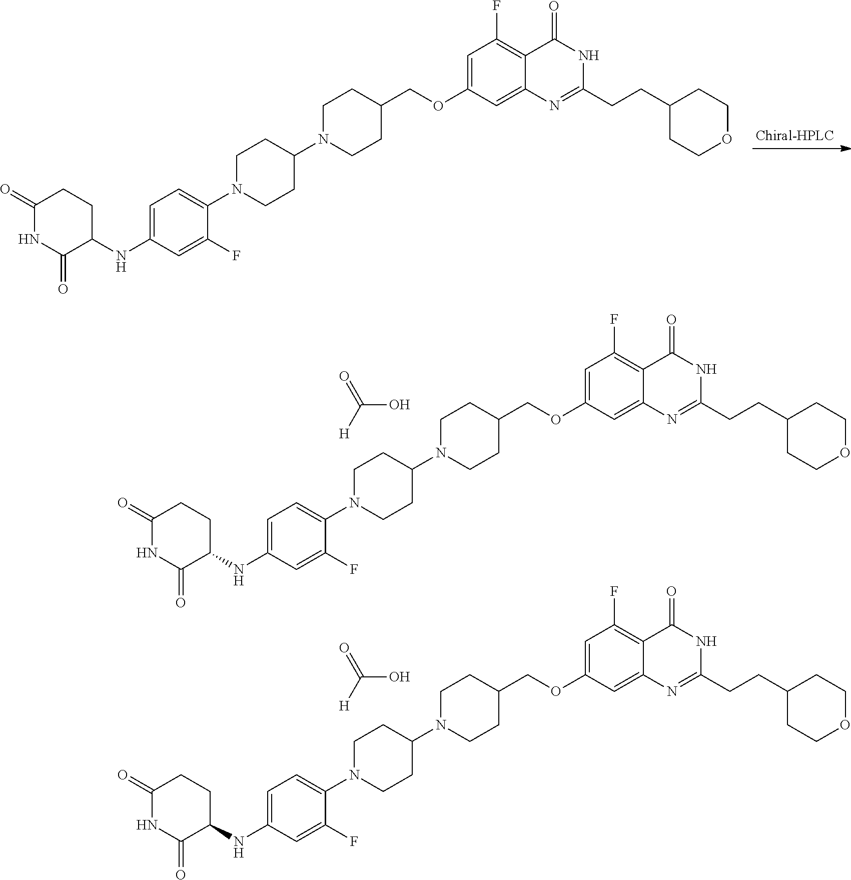

200 mg of the racemic 3-((3-fluoro-4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidin]-11′-yl)phenyl)amino)piperidine-2,6-dione (Example 117) was separated by Chiral-Prep-HPLC with the following conditions (Column: CHIRALPAK IF, 2*25 cm, 5 μm; Mobile Phase A: Hex (0.5% 2 M NH₃-MeOH)-HPLC, Mobile Phase B: EtOH:DCM=1:1-HPLC; Flow rate: 20 mL/min; Gradient: isocratic 80% B; Wavelength: 220/254 nm; RT (min): 15.09 (Example 117a); RT (min): 16.79 (Example 117b); Sample Solvent: EtOH:DCM=1:1-HPLC; Injection Volume: 1.2 mL; Number of runs: 4). The fractions were directly concentrated and then lyophilized to afford the title compounds. Absolute stereochemistry was not assigned.

Example 117a: The first eluting isomer isolated using the above conditions (77 mg) as a white solid. LCMS (ESI, m/z): 693.35 [M+H]⁺. 1H NMR (400 MHz, DMSO-d₆) δ 12.04 (s, 1H), 10.77 (s, 1H), 8.23 (s, 1H), 6.96-6.75 (m, 3H), 6.50 (dd, J=14.8, 2.1 Hz, 1H), 6.41 (dd, J=8.7, 1.7 Hz, 1H), 5.79 (d, J=7.6 Hz, 1H), 4.29-4.21 (m, 1H), 3.97 (d, J=5.9 Hz, 2H), 3.89-3.77 (m, 2H), 3.25 (d, J=11.7 Hz, 3H), 3.17 (d, J=10.7 Hz, 2H), 2.93 (d, J=10.3 Hz, 2H), 2.74-2.68 (m, 1H), 2.62-2.53 (m, 4H), 2.36-2.31 (m, 1H), 2.20 (t, J=11.3 Hz, 2H), 2.11-2.02 (m, 1H), 1.95-1.70 (m, 6H), 1.70-1.51 (m, 6H), 1.52-1.45 (m, 1H) 1.33-1.11 (m, 4H). Chiral HPLC: Column: CHIRALPAK IF-3; Mobile Phase A: Hex (0.2% DEA):(EtOH:DCM=1:1)=20:80; Flow rate: 1.0 mL/min; Retention time: 1.46 min (faster peak).

Example 117b: The second eluting isomer isolated using the above conditions (33 mg) as an off-white solid. LCMS (ESI, m/z): 693.35 [M+H]⁺. 1H NMR (400 MHz, DMSO-d₆) δ 12.05 (s, 1H), 10.77 (s, 1H), 8.20 (s, 1H), 6.95-6.79 (m, 3H), 6.50 (dd, J=15.0, 2.6 Hz, 1H), 6.41 (dd, J=8.7, 2.6 Hz, 1H), 5.79 (d, J=7.7 Hz, 1H), 4.29-4.22 (m, 1H), 3.98 (d, J=5.8 Hz, 2H), 3.87-3.80 (m, 2H), 3.30-3.17 (m, 5H), 2.95 (d, J=10.7 Hz, 2H), 2.77-2.69 (m, 1H), 2.61-2.59 (m, 1H), 2.58-2.54 (m, 3H), 2.38-2.33 (m, 1H), 2.23 (t, J=11.2 Hz, 2H), 2.13-2.05 (m, 1H), 1.95-1.70 (m, 6H), 1.70-1.53 (m, 6H), 1.53-1.45 (m, 1H), 1.38-1.26 (m, 2H), 1.24-1.11 (m, 2H). Chiral HPLC: Column: CHIRALPAK IF-3; Mobile Phase A: Hex (0.2% DEA):(EtOH:DCM=1:1)=20:80; Flow rate: 1.0 mL/min; Retention time: 3.08 min (slower peak).

### Example 118: Synthesis of 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-1-yl)phenyl)amino)piperidine-2,6-dione

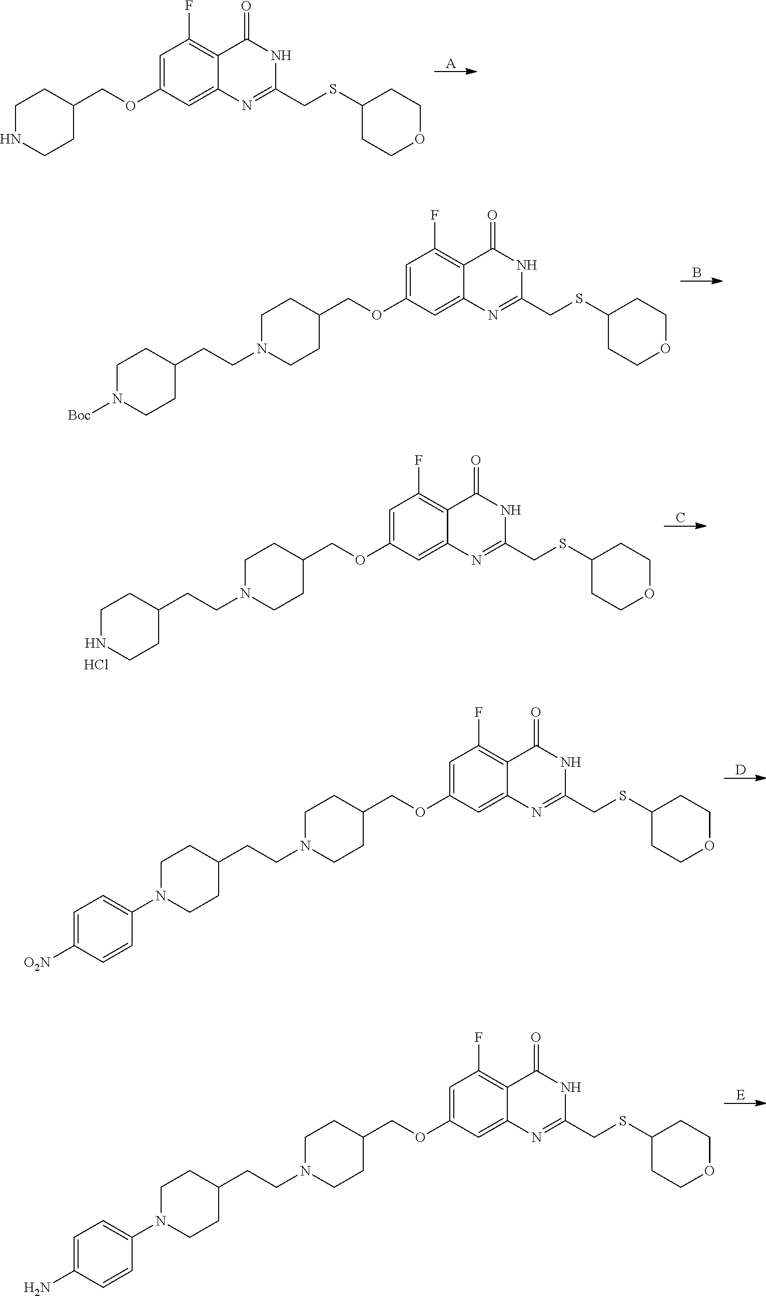

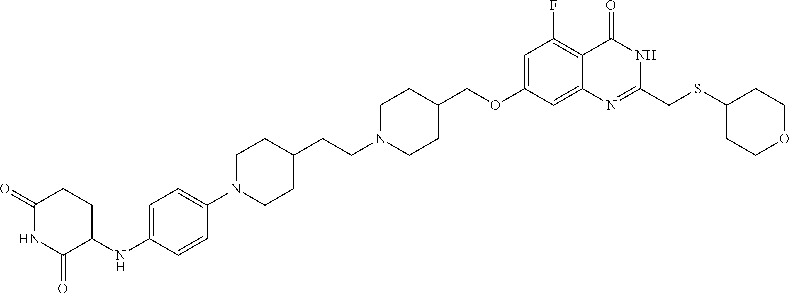 [SMILES: O=C1CCC(Nc2ccc(N3CCC(CCN4CCC(COc5cc(F)c6c(=O)[nH]c(CSC7CCOCC7)nc6c5)CC4)CC3)cc2)C(=O)N1]

### Step A

A solution of 5-fluoro-7-(piperidin-4-ylmethoxy)-2-(((tetrahydro-2H-pyran-4-yl)thio)methyl)quinazolin-4(3H)-one (2.00 g, 4.91 mmol, 1 equiv), tert-butyl 4-(2-bromoethyl)piperidine-1-carboxylate (2.87 g, 9.82 mmol, 2 equiv) and DIEA (1.90 g, 14.7 mmol, 3 equiv) in DMSO (12 mL) was stirred for 5 hours at 80° C. The residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. The result in tert-butyl 4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidine-1-carboxylate (1.08 g, 33%) as a white solid. LCMS (ESI, m/z): 619.30 [M+H]⁺.

### Step B

A solution of tert-butyl 4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidine-1-carboxylate (1.08 g, 1.75 mmol, 1 equiv) in HCl in 1,4-dioxane (10 mL, 4 M) was stirred for 1 hour. The resulting mixture was concentrated under vacuum to afford crude 5-fluoro-7-((1-(2-(piperidin-4-yl)ethyl)piperidin-4-yl)methoxy)-2-(((tetrahydro-2H-pyran-4-yl)thio)methyl)quinazolin-4(3H)-one hydrochloride (1.1 g) as a grey solid. The crude product was used in the next step without further purification. LCMS (ESI, m/z): 519.20 [M+H]⁺.

### Step C

A solution of 5-fluoro-7-((1-(2-(piperidin-4-yl)ethyl)piperidin-4-yl)methoxy)-2-(((tetrahydro-2H-pyran-4-yl)thio)methyl)quinazolin-4(3H)-one hydrochloride (1.10 g, 2.12 mmol, 1 equiv), 1-fluoro-4-nitrobenzene (0.449 g, 3.18 mmol, 1.5 equiv) and K₂CO₃ (0.88 g, 6.36 mmol, 3 equiv) in DMF (5 mL) was stirred for 5 hours at 80° C. The reaction was quenched with water and extracted with EtOAc (3×10 mL). The resulting mixture was washed with brine (3×30 mL). The combined organic layers were concentrated under reduced pressure. The residue was purified by silica gel column chromatography, eluting with DCM/MeOH (10:1) to afford 5-fluoro-7-((1-(2-(1-(4-nitrophenyl)piperidin-4-yl)ethyl)piperidin-4-yl)methoxy)-2-(((tetrahydro-2H-pyran-4-yl)thio)methyl)quinazolin-4(3H)-one (790 mg, 58%) as a yellow solid. LCMS (ESI, m/z): 640.20 [M+H]⁺.

### Step D

A solution of 5-fluoro-7-((1-(2-(1-(4-nitrophenyl)piperidin-4-yl)ethyl)piperidin-4-yl)methoxy)-2-(((tetrahydro-2H-pyran-4-yl)thio)methyl)quinazolin-4(3H)-one (790 mg, 1.25 mmol, 1 equiv), Fe (349 mg, 6.25 mmol, 5 equiv) and NH₄Cl (134 mg, 2.50 mmol, 2 equiv) in EtOH (5 mL) and water (1 mL) was stirred for 5 hours at 80° C. The resulting mixture was filtered and the filter cake was washed with EtOH (5×10 mL). The filtrate was concentrated under reduced pressure. The residue was purified by silica gel column chromatography, eluting with DCM/MeOH (8:1) to afford 7-((1-(2-(1-(4-aminophenyl)piperidin-4-yl)ethyl)piperidin-4-yl)methoxy)-5-fluoro-2-(((tetrahydro-2H-pyran-4-yl)thio)methyl)quinazolin-4(3H)-one (355 mg, 42%) as a brown solid. LCMS (ESI, m/z): 610.35 [M+H]⁺.

### Step E

A solution of 7-((1-(2-(1-(4-aminophenyl)piperidin-4-yl)ethyl)piperidin-4-yl)methoxy)-5-fluoro-2-(((tetrahydro-2H-pyran-4-yl)thio)methyl)quinazolin-4(3H)-one (165 mg, 0.271 mmol, 1 equiv), 3-bromopiperidine-2,6-dione (208 mg, 1.08 mmol, 4 equiv) and NaHCO₃ (114 mg, 1.36 mmol, 5 equiv) in ACN (4 mL) was stirred overnight at 90° C. The residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm to give 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-1-yl)phenyl)amino)piperidine-2,6-dione (39 mg, 19%) as a dark blue solid. LCMS (ESI, m/z): 721.25 [M+H]⁺. ¹H NMR (300 MHz, DMSO-d₆) δ 12.16 (s, 1H), 10.73 (s, 1H), 6.95 (s, 1H), 6.83 (s, 1H), 6.76 (d, J=8.2 Hz, 2H), 6.66 (dd, J=8.2 Hz, 2H), 5.35 (d, J=7.1 Hz, 1H), 4.21-4.12 (m, 1H), 4.02-3.95 (m, 2H), 3.85 (d, J=11.5 Hz, 2H), 3.63 (s, 2H), 3.30-3.26 (m, 6H), 3.16-3.02 (m, 2H), 2.80-2.60 (m, 5H), 2.55-2.50 (m, 2H), 2.15-2.06 (m, 1H), 1.95-1.68 (m, 7H), 1.65-0.81 (m, 10H).

Example 119 was synthesized according to the procedure described for the synthesis 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-1-yl)phenyl)amino)piperidine-2,6-dione (Example 118) using appropriate building blocks and modified reaction conditions (such as reagents, reagent ratio, temperature, and reaction time) and purification conditions as needed.

| Example | Structure | Analytical Data |
|---|---|---|
| 119 | 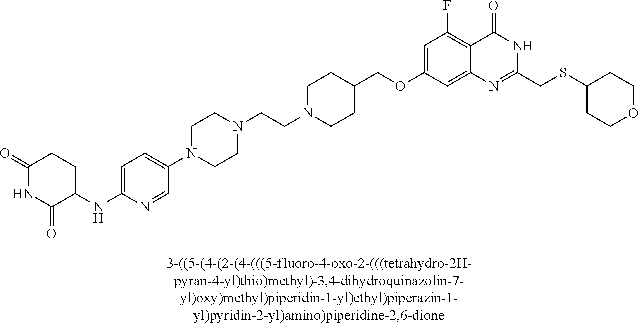 [SMILES: O=C1CCC(Nc2ccc(N3CCN(CCN4CCC(COc5cc(F)c6c(=O)[nH]c(CSC7CCOCC7)nc6c5)CC4)CC3)cn2)C(=O)N1] | LCMS (ESI, m/z): 723.25 [M + H]⁺; ¹H NMR (400 MHz, DMSO-d₆) δ 12.17 (s, 1H), 10.73 (s, 1H), 7.65 (s, 1H), 7.23 (dd, J = 4.0, 8.0 Hz, 1H), 6.90 (d, J = 11.6 Hz, 2H), 6.56 (d, J = 9.0 Hz, 1H), 6.41 (d, J = 7.5 Hz, 1H), 4.70-4.52 (m, 1H), 4.00 (s, 2H), 3.86-3.79 (m, 2H), 3.63 (s, 2H), 3.52- 3.51 (m, 1H), 3.31-3.28 (m, 6H), 3.10-3.02 (m, 2H), 3.00-2.85 (m, 5H), 2.80-2.69 (m, 2H), 2.58-2.50 (m, 3H), 2.13-2.09 (m, 1H), 2.05-1.85 (m, 5H), |
| | | 1.80-1.70 (m, 2H), |
| | | 1.50-1.28 (m, 4H). |

### Example 120: Synthesis of 3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)oxy)phenyl)amino)piperidine-2,6-dione

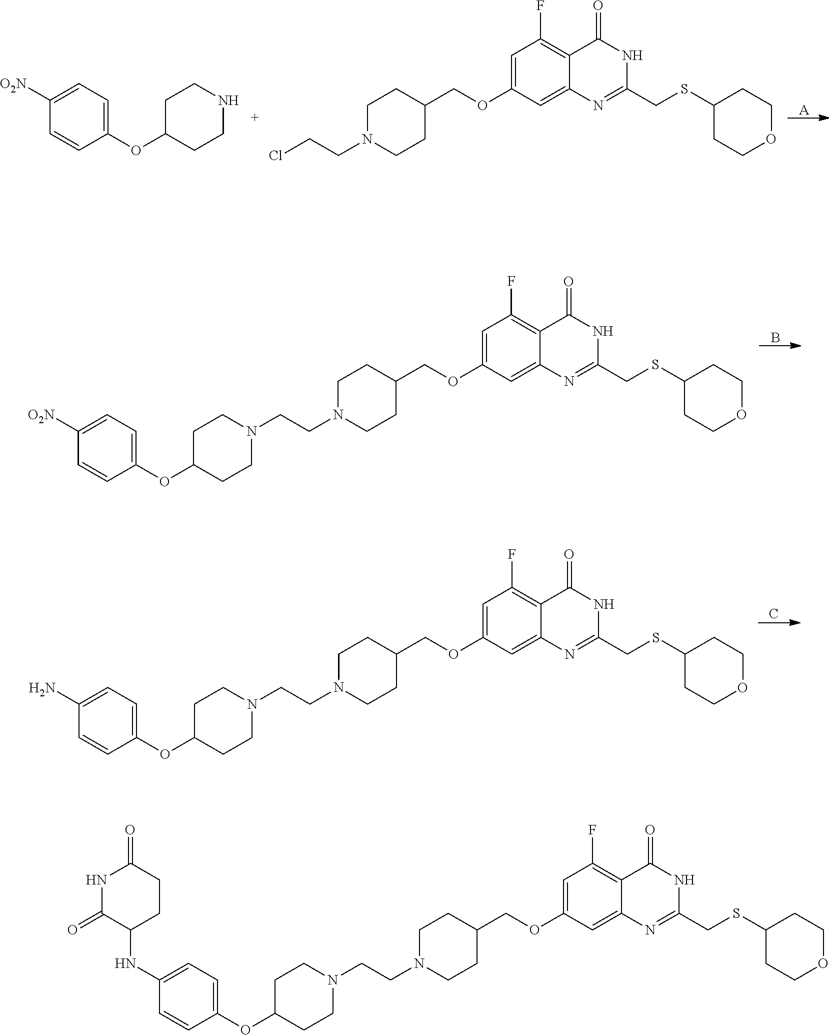

### Step A

A solution of 4-(4-nitrophenoxy)piperidine (1.0 g, 4.50 mmol, 1 equiv), 7-((1-(2-chloroethyl)piperidin-4-yl)methoxy)-5-fluoro-2-(((tetrahydro-2H-pyran-4-yl)thio)methyl)quinazolin-4(3H)-one (2.33 g, 4.95 mmol, 1.1 equiv) and DIEA (1.74 g, 13.5 mmol, 3 equiv) in DMSO (12 mL) was stirred for 2 hours at 80° C. The residue was purified by reverse-phase flash chromatography with the following conditions: column, C18 silica gel; mobile phase, MeCN in water (0.1% FA), 10% to 40% gradient in 10 min; detector, UV 254 nm to give 5-fluoro-7-((1-(2-(4-(4-nitrophenoxy)piperidin-1-yl)ethyl)piperidin-4-yl)methoxy)-2-(((tetrahydro-2H-pyran-4-yl)thio)methyl)quinazolin-4(3H)-one (1.5 g, 51%) as a brown oil. LCMS (ESI, m/z): 656.15 [M+H]⁺.

### Step B

To a stirred solution of 5-fluoro-7-((1-(2-(4-(4-nitrophenoxy)piperidin-1-yl)ethyl)piperidin-4-yl)methoxy)-2-(((tetrahydro-2H-pyran-4-yl)thio)methyl)quinazolin-4(3H)-one (1.0 g, 1.53 mmol, 1 equiv) in EtOH (10 mL) was added Fe (0.34 g, 6.10 mmol, 4 equiv), NH₄Cl (0.33 g, 6.10 mmol, 4 equiv) and water (2 mL) at room temperature. The resulting mixture was stirred for 4 hours at 80° C. The resulting mixture was filtered through a Celite pad, the filter cake was washed with hot ethanol. The filtrate was concentrated under reduced pressure. The residue was purified by reverse-phase flash chromatography with the following conditions: column, C18 silica gel; mobile phase, MeCN in water (10 mmol/L NH₄HCO₃), 10% to 80% gradient in 20 min; detector, UV 254 nm to give 7-((1-(2-(4-(4-aminophenoxy)piperidin-1-yl)ethyl)piperidin-4-yl)methoxy)-5-fluoro-2-(((tetrahydro-2H-pyran-4-yl)thio)methyl)quinazolin-4(3H)-one (530 mg, 56%) as a brown solid. LCMS (ESI, m/z): 626.20 [M+H]⁺.

### Step C

A solution of 7-((1-(2-(4-(4-aminophenoxy)piperidin-1-yl)ethyl)piperidin-4-yl)methoxy)-5-fluoro-2-(((tetrahydro-2H-pyran-4-yl)thio)methyl)quinazolin-4(3H)-one (314 mg, 0.502 mmol, 1 equiv), NaHCO₃ (126 mg, 1.51 mmol, 3.0 equiv) and 3-bromopiperidine-2,6-dione (241 mg, 1.26 mmol, 2.5 equiv) in ACN (8 mL) was stirred overnight at 80° C. The resulting mixture was concentrated under reduced pressure. The residue was purified by reverse-phase flash chromatography with the following conditions: column, C18 silica gel; mobile phase, MeCN in water (0.1% FA), 10% to 50% gradient in 35 min; detector, UV 254 nm. The resulting impure product (208 mg) was further purified by Prep-HPLC with the following conditions (Column: XBridge Prep Phenyl Column, 19*250 mm, 5 μm; Mobile Phase A: water (10 mmol/L NH₄HCO₃), Mobile Phase B: ACN; Flow rate: 25 mL/min; Gradient: 25% B to 40% B in 9 min; Wave Length: 254 nm/220 nm; RT (min): 9.0) to afford 3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)oxy)phenyl)amino)piperidine-2,6-dione (18 mg, 5%) as a white solid. LCMS (ESI, m/z): 776.20 [M+H]⁺. ¹H NMR (300 MHz, DMSO-d₆) δ 12.11 (s, 1H), 10.74 (s, 1H), 6.89-6.82 (m, 2H), 6.73 (dd, J=9.1, 2.3 Hz, 2H), 6.66 (dd, J=9.1, 2.3 Hz, 2H), 5.44 (d, J=7.1 Hz, 1H), 4.25-4.13 (m, 2H), 4.10-4.02 (m, 2H), 3.82 (d, J=11.5 Hz, 3H), 3.62 (s, 2H), 3.11-3.02 (m, 1H), 2.89 (d, J=10.9 Hz, 2H), 2.76-2.58 (m, 4H), 2.45-2.37 (m, 4H), 2.21-2.06 (m, 3H), 1.92-1.75 (m, 7H), 1.72 (d, J=11.1 Hz, 3H), 1.60-1.40 (m, 4H), 1.35-1.21 (m, 3H).

Examples 121-123 were synthesized according to the procedure described for the synthesis 3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)oxy)phenyl)amino)piperidine-2,6-dione (Example 120) using appropriate building blocks and modified reaction conditions (such as reagents, reagent ratio, temperature, and reaction time) and purification conditions as needed.

| Example | Structure | Analytical Data |
|---|---|---|
| 121 | 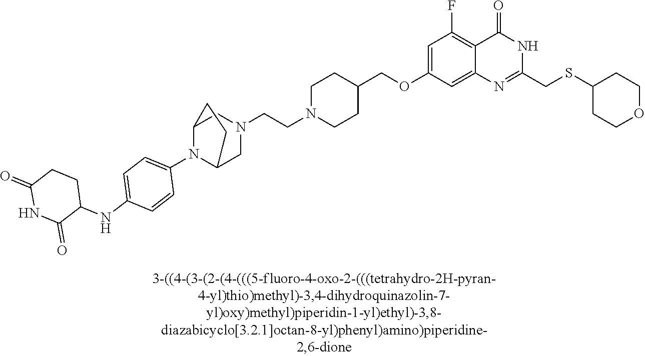 [SMILES: O=C1CCC(Nc2ccc(N3C4CCC3CN(CCN3CCC(COc5cc(F)c6c(=O)[nH]c(CSC7CCOCC7)nc6c5)CC3)C4)cc2)C(=O)N1] | LCMS (ESI, m/z): 748.45 [M + H]⁺; ¹H NMR (400 MHz, DMSO-d₆) δ 12.16 (s, 1H), 10.75 (s, 1H), 8.18 (s, 2H, HCOOH), 6.92-6.79 (m, 2H), 6.69-6.46 (m, 4H), 5.30-5.10 (m, 1H), 4.17- 4.10 (m, 1H), 4.06- 3.91 (m, 4H), 3.86-3.78 (m, 2H), 3.62 (s, 2H), 3.37-3.29 (m, 3H), 3.10-3.01 (m, 2H), 2.99- 2.88 (m, 2H), 2.78- 2.68 (m, 1H), 2.62-2.41 (m, 4H), 2.38-2.34 (m, 3H), 2.18- |
| | | 2.01 (m, 3H), 1.99- |
| | | 1.52 (m, 10H), |
| | | 1.51-1.38 (m, |
| | | 2H), 1.35-1.25 |
| | | (m, 2H). |
| 122 | 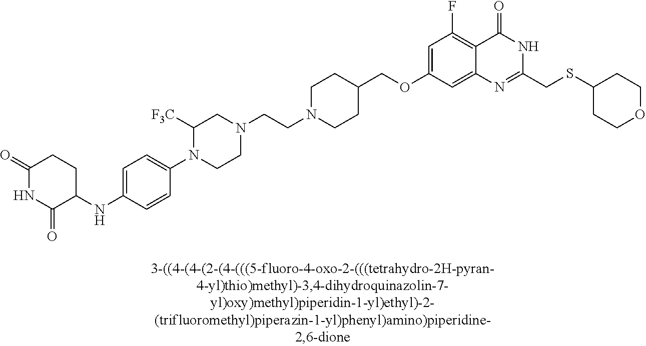 [SMILES: O=C1CCC(Nc2ccc(N3CCN(CCN4CCC(COc5cc(F)c6c(=O)[nH]c(CSC7CCOCC7)nc6c5)CC4)CC3C(F)(F)F)cc2)C(=O)N1] | LCMS (ESI, m/z): 790.25 [M + H]⁺; ¹H NMR (400 MHz, DMSO-d₆) δ 12.13 (s, 1H), 10.74 (s, 1H), 6.93- 6.85 (m, 2H), 6.85 (d, J = 8.8 Hz, 2H), 6.66 (d, J = 6.8 Hz, 2H), 5.43 (d, 6.8 Hz, 1H), 4.49-4.37 (m, 1H), 4.24-4.14 (m , 1H), 3.98 (d, J = 5.9 Hz, 2H), 3.87-3.77 (m, 2H), 3.62 (s, 2H), 3.39-3.32 (m, 2H), 3.27-3.21 (m, 1H), 3.14- 3.01 (m, 3H), 2.98- |
| | | 2.89 (m, 2H), |
| | | 2.89-2.80 (m, |
| | | 1H), 2.79-2.65 |
| | | (m, 1H), 2.63- |
| | | 2.58 (m, 1H), 2.58- |
| | | 2.54 (m, 1H), |
| | | 2.46-2.37 (m, |
| | | 3H), 2.36-2.30 |
| | | (m, 1H), 2.23- |
| | | 2.06 (m, 2H), 2.05- |
| | | 1.93 (m, 2H), |
| | | 1.92-1.81 (m, |
| | | 3H), 1.81-1.66 |
| | | (m, 3H), 1.54- |
| | | 1.39 (m, 2H), 1.38- |
| | | 1.19 (m, 2H). |
| 123 | 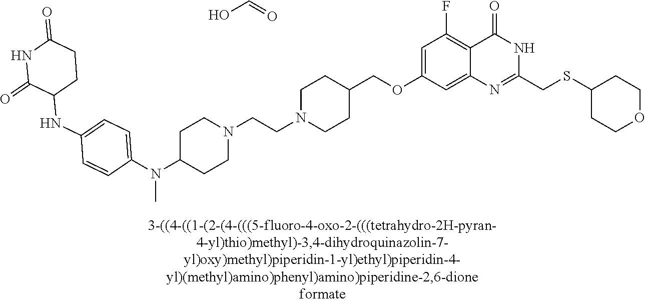 [SMILES: CN(c1ccc(NC2CCC(=O)NC2=O)cc1)C1CCN(CCN2CCC(COc3cc(F)c4c(=O)[nH]c(CSC5CCOCC5)nc4c3)CC2)CC1 ; O=CO] | LCMS (ESI, m/z): 750.30 [M + H]⁺. ¹H NMR (400 MHz, DMSO-d₆) δ 12.13 (s, 1H), 10.73 (s, 1H), 8.21 (s, 1H), 6.88 (s, 1H), 6.87 (d, J = 10.4 Hz, 1H), 6.71 (d, J = 8.6 Hz, 2H), 6.61 (d, J = 8.6 Hz, 2H), 5.28 (br, 1H), 4.16 (dd, J = 11.0, 4.7 Hz, 1H), 3.98 (d, J = 5.9 Hz, 2H), 3.85-3.78 (m, 2H), 3.62 (s, 2H), 3.35-3.29 (m, 2H), 3.23-3.17 |
| | | (m, 1H), 3.10- |
| | | 3.02 (m, 1H), 2.97- |
| | | 2.91 (m, 4H), 2.78- |
| | | 2.65 (m, 1H), |
| | | 2.64-2.54 (m, |
| | | 4H), 2.47 (s, 3H), |
| | | 2.19-1.95 (m, |
| | | 5H), 1.93-1.82 |
| | | (m, 3H), 1.81- |
| | | 1.68 (m, 3H), 1.66- |
| | | 1.52 (m, 4H), |
| | | 1.51-1.39 (m, |
| | | 2H), 1.35-1.24 |
| | | (m, 2H). |

Examples 124-126 were synthesized according to the procedure described for the synthesis 3-((4-(4-(3-(((5-fluoro-4-oxo-2-(((1-(2,2,2-trifluoroethyl)piperidin-4-yl)thio)methyl)-3,4-dihydroquinazolin-7-yl)oxy)methyl)cyclobutyl)piperazin-1-yl)phenyl)amino)piperidine-2,6-dione (Example 56) using appropriate building blocks and modified reaction conditions (such as reagents, reagent ratio, temperature, and reaction time) and purification conditions as needed.

| Example | Structure | Analytical Data |
|---|---|---|
| 124 | 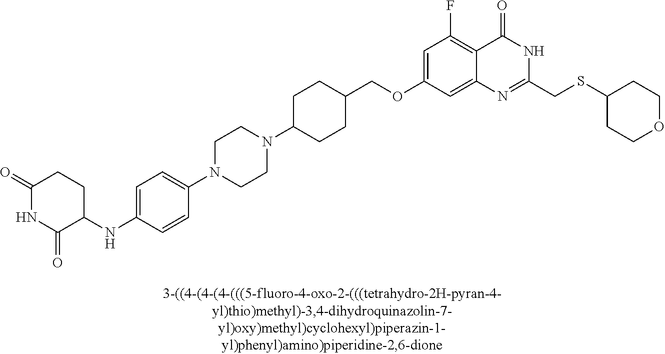 [SMILES: O=C1CCC(Nc2ccc(N3CCN(C4CCC(COc5cc(F)c6c(=O)[nH]c(CSC7CCOCC7)nc6c5)CC4)CC3)cc2)C(=O)N1] | LCMS (ESI, m/z): 693.25 [M + H]⁺; ¹H NMR (400 MHz, DMSO-d₆) δ 12.06 (s, 1H), 10.73 (s, 1H), 6.89 (d, J = 18.1 Hz, 2H), 6.75 (d, J = 7.0 Hz, 2H), 6.61 (d, J = 7.0 Hz, 2H), 5.34 (s, 1H), 4.21-4.15 (m, 1H), 4.04 (d, J = 6.8 Hz, 2H), 3.82 (d, J = 11.2 Hz, 2H), 3.62 (s, 2H), 3.10-3.01 (m, 1H), 2.94-2.85 (m, 3H), 2.81- 2.67 (m, 2H), 2.58- 2.50 (m, 3H), 2.21- 2.15 (m, 1H), 2.12- 2.01 (m, 1H), 2.05- |
| | | 1.95 (m, 2H), |
| | | 1.94-1.79 (m, |
| | | 3H), 1.78-1.33 |
| | | (m, 10H), 1.31- |
| | | 1.15 (m, 3H). |
| 125 | 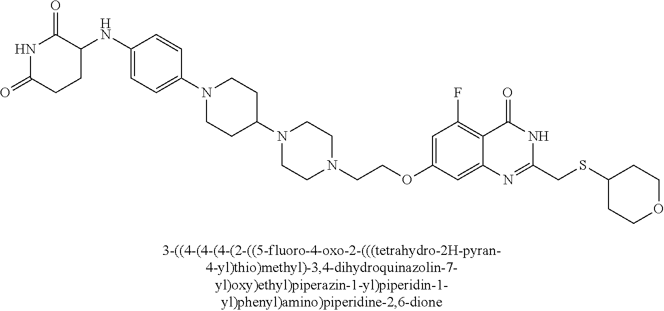 [SMILES: O=C1CCC(Nc2ccc(N3CCC(N4CCN(CCOc5cc(F)c6c(=O)[nH]c(CSC7CCOCC7)nc6c5)CC4)CC3)cc2)C(=O)N1] | LCMS (ESI, m/z): 708.25 [M + H]⁺; ¹H NMR (300 MHz, DMSO-d₆) δ 12.23 (br, 1H), 10.80 (s, 1H), 7.38-7.10 (m, 2H), 7.01-6.90 (m, 2H), 6.75 (d, J = 8.5 Hz, 2H), 4.59- 4.19 (m, 4H), 3.83 (dt, J = 11.5, 3.7 Hz, 3H), 3.64 (s, 2H), 3.62-3.56 (m, 6H), 3.39-. 31 (m, 6H), 3.19-3.05 (m, 4H), 2.79- 2.60 (m, 3H), 2.21- 2.01 (m, 3H), 1.98- 1.70 (m, 5H), 1.53- 1.42 (m, 2H). |
| 126 | 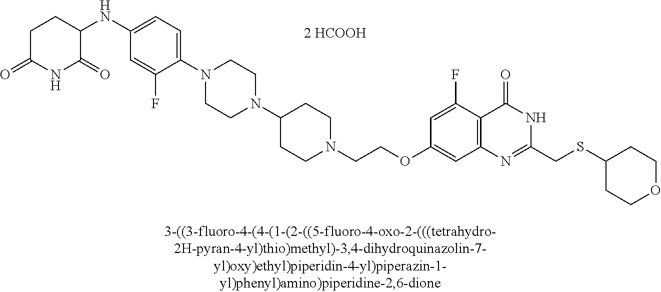 [SMILES: O=C1CCC(Nc2ccc(N3CCN(C4CCN(CCOc5cc(F)c6c(=O)[nH]c(CSC7CCOCC7)nc6c5)CC4)CC3)c(F)c2)C(=O)N1] | LCMS (ESI, m/z): 726.25 [M + H]⁺; ¹H NMR (400 MHz, DMSO-d₆) δ 12.17 (s, 1H), 10.77 (s, 1H), 8.17 (s, 2H), 6.94-6.87 (m, 2H), 6.82 (t, J = 10.0 Hz, 1H), 6.55-6.48 (m, 1H), 6.45-6.39 (m, 1H), 5.80 (d, J = 7.6 Hz, 1H), 4.30- 4.19 (m, 3H), 3.86- 3.77 (m, 2H), 3.38- 3.29 (m, 2H), 3.11- 2.97 (m, 3H), 2.85 (t, J = 4.6 Hz, 4H), 2.78-2.68 |
| | | (m, 3H), 2.67-2.56 |
| | | (m, 5H), 2.56- |
| | | 2.52 (m, 2H), 2.31- |
| | | 2.19 (m, 1H), 2.14- |
| | | 1.99 (m, 3H), 1.94- |
| | | 1.82 (m, 3H), |
| | | 1.81-1.73 (m, |
| | | 2H), 1.52-1.38 (m, |
| | | 4H). |

### Example 127: Synthesis of 3-((4-(3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidin-1-yl)phenyl)amino)piperidine-2,6-dione

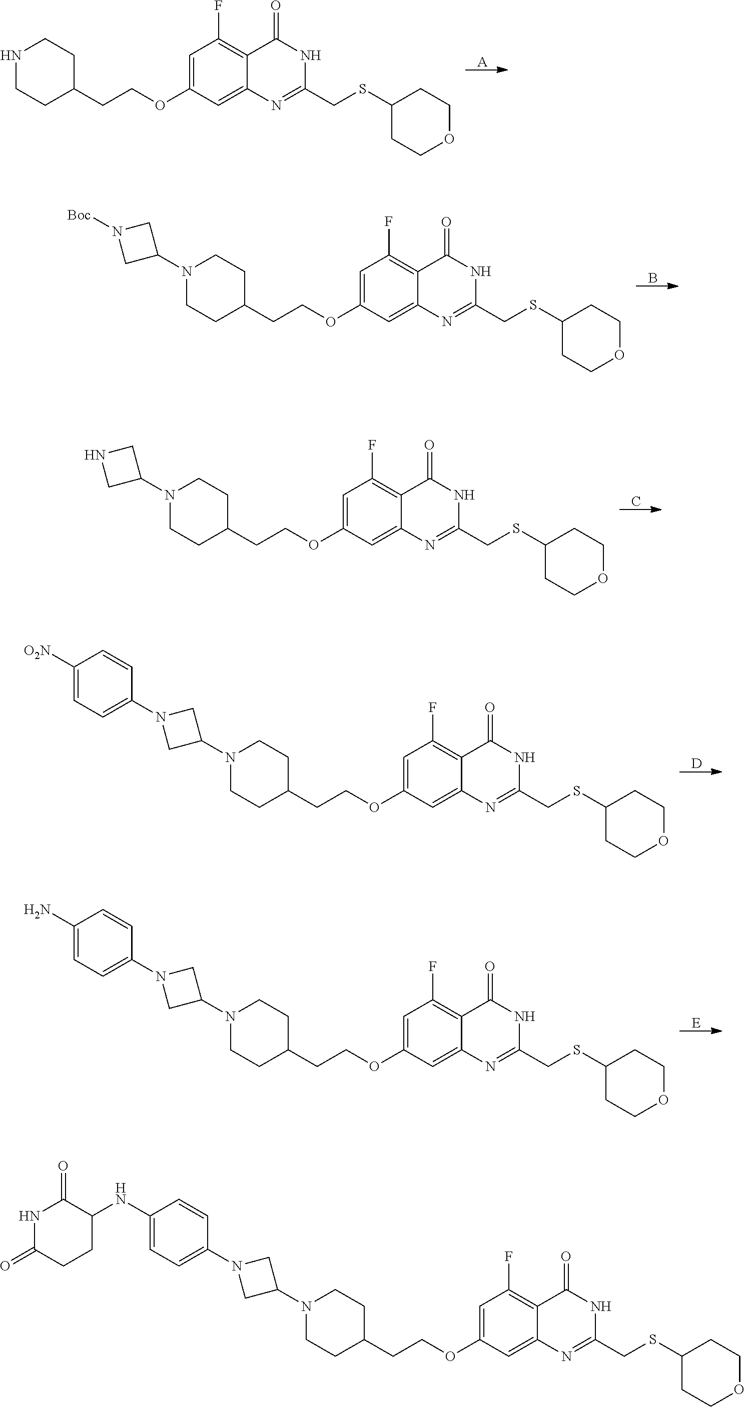

### Step A

A solution of 5-fluoro-7-(2-(piperidin-4-yl)ethoxy)-2-(((tetrahydro-2H-pyran-4-yl)thio)methyl)quinazolin-4(3H)-one (0.85 g, 2.02 mmol, 1 equiv), STAB (1.28 g, 6.05 mmol, 3.0 equiv) and tert-butyl 3-oxoazetidine-1-carboxylate (0.69 g, 4.1 mmol, 2.0 equiv) in DCM (15 mL) was stirred for 2 hours. The resulting mixture was concentrated under vacuum. The residue was purified by reverse-phase flash chromatography with the following conditions: column, C18 silica gel; mobile phase, MeCN in water (10 mmol/L NH₄HCO₃), 10% to 48% gradient in 10 mi; detector, UV 254 nm to give tert-butyl 3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidine-1-carboxylate (530 mg, 46%) as an off-white solid. LCMS (ESI, m/z): 577.20 [M+H]⁺.

### Step B

A solution of tert-butyl 3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidine-1-carboxylate (530 mg, 0.92 mmol, 1 equiv) in HCl in 1,4-dioxane (6 mL, 4M) and MeOH (6 mL) was stirred for 2 hours. The resulting mixture was concentrated under vacuum to afford 7-(2-(1-(azetidin-3-yl)piperidin-4-yl)ethoxy)-5-fluoro-2-(((tetrahydro-2H-pyran-4-yl)thio)methyl)quinazolin-4(3H)-one (620 mg, 99%) as off-white solid. LCMS (ESI, m/z): 477.15 [M+H]⁺.

### Step C

A solution of 7-(2-(1-(azetidin-3-yl)piperidin-4-yl)ethoxy)-5-fluoro-2-(((tetrahydro-2H-pyran-4-yl)thio)methyl)quinazolin-4(3H)-one (200 mg, 0.42 mmol, 1 equiv), 1-fluoro-4-nitrobenzene (59.2 mg, 0.42 mmol, 1 equiv) and DIEA (163 mg, 1.26 mmol, 3 equiv) in NMP (5 mL) was stirred for 1 hour at 80° C. The resulting mixture was concentrated under reduced pressure. The residue was purified by silica gel column chromatography, eluting with DCM/MeOH (5:1) to afford 5-fluoro-7-(2-(1-(1-(4-nitrophenyl)azetidin-3-yl)piperidin-4-yl)ethoxy)-2-(((tetrahydro-2H-pyran-4-yl)thio)methyl)quinazolin-4(3H)-one (230 mg, 92%) as a yellow solid. LCMS (ESI, m/z): 598.35 [M+H]⁺.

### Step D

A solution of 5-fluoro-7-(2-(1-(1-(4-nitrophenyl)azetidin-3-yl)piperidin-4-yl)ethoxy)-2-(((tetrahydro-2H-pyran-4-yl)thio)methyl)quinazolin-4(3H)-one (230 mg, 0.39 mmol, 1 equiv), NH₄Cl (41 mg, 0.77 mmol, 2 equiv) and Fe (107 mg, 1.93 mmol, 5 equiv) in water (1 mL) and EtOH (5 mL) was stirred for 2 hours at 80° C. The resulting mixture was filtered, the filter cake was washed with EtOH (3×10 mL). The filtrate was concentrated under reduced pressure. This resulted in 7-(2-(1-(1-(4-aminophenyl)azetidin-3-yl)piperidin-4-yl)ethoxy)-5-fluoro-2-(((tetrahydro-2H-pyran-4-yl)thio)methyl)quinazolin-4(3H)-one (120 mg, 55%) as a yellow solid. LCMS (ESI, m/z): 568.45 [M+H]⁺.

### Step E

A solution of 7-(2-(1-(1-(4-aminophenyl)azetidin-3-yl)piperidin-4-yl)ethoxy)-5-fluoro-2-(((tetrahydro-2H-pyran-4-yl)thio)methyl)quinazolin-4(3H)-one (100 mg, 0.18 mmol, 1 equiv), 3-bromopiperidine-2,6-dione (101 mg, 0.53 mmol, 3 equiv) and DIEA (68.3 mg, 0.53 mmol, 3 equiv) in NMP (3 mL) was stirred for 1 hour at 80° C. The resulting mixture was concentrated under reduced pressure. The residue was purified by silica gel column chromatography, eluting with DCM/EtOH (5:1) to afford 3-((4-(3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidin-1-yl)phenyl)amino)piperidine-2,6-dione (5.6 mg, 5%) as a green solid. LCMS (ESI, m/z): 679.35 [M+H]⁺. ¹H NMR (400 MHz, DMSO-d₆) δ 12.14 (s, 1H), 10.73 (s, 1H), 6.88 (d, J=13.2 Hz, 2H), 6.61 (d, J=8.5 Hz, 2H), 6.30 (d, J=8.4 Hz, 2H), 5.18 (d, J=7.1 Hz, 1H), 4.19-4.09 (m, 3H), 3.88-3.78 (m, 4H), 3.62 (s, 2H), 3.38-3.33 (m, 2H) 3.11-3.01 (m, 3H), 2.82-2.65 (m, 4H), 2.18-2.08 (m, 1H), 1.91-1.79 (m, 5H), 1.78-1.63 (m, 6H), 1.53-1.38 (m, 3H), 1.25-1.11 (m, 1H).

Example 128 synthesized according to the procedure described for the synthesis 3-((4-(3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidin-1-yl)phenyl)amino)piperidine-2,6-dione (Example 127) using appropriate building blocks and modified reaction conditions (such as reagents, reagent ratio, temperature, and reaction time) and purification conditions as needed.

| Example | Structure | Analytical Data |
|---|---|---|
| 128 | 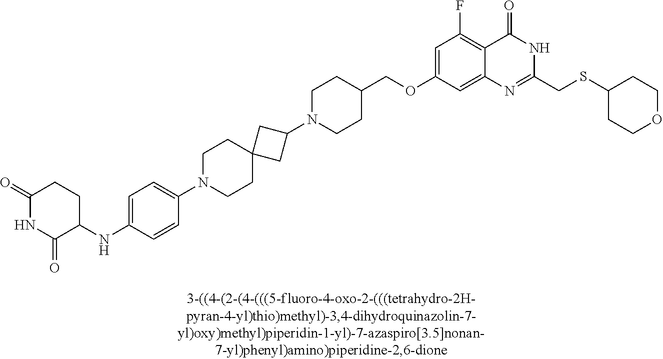 [SMILES: O=C1CCC(Nc2ccc(N3CCC4(CC3)CC(N3CCC(COc5cc(F)c6c(=O)[nH]c(CSC7CCOCC7)nc6c5)CC3)C4)cc2)C(=O)N1] | LCMS (ESI, m/z): 733.45 [M + H]⁺; ¹H NMR (400 MHz, Methanol-d₄) δ 7.44 (d, J = 9.2 Hz, 2H), 6.97 (d, J = 2.4 Hz, 1H), 6.93-6.79 (m, 3H), 4.41 (dd, J = 12.0, 4.9 Hz, 1H), 4.10 (d, J = 5.4 Hz, 2H), 3.93 (m, 2H), 3.83-3.72 (m, 1H), 3.68-3.52 (m, 6H), 3.45-3.41 (m, 2H), 3.16-3.12 (m, 1H), 3.06-2.93 (m, 1H), 2.98-2.70 (m, 4H), 2.58-2.42 (m, 2H), 2.36-1.91 (m, 14H), 1.80-1.70 (m, 2H), 1.61-1.51 (m, 2H). |

### Example 129: Synthesis of 3-((4-(4-(2-(4-(((5,6-difluoro-4-oxo-2-(((tetrahydro-2H-pyran-4-yl)thio)methyl)-3,4-dihydroquinazolin-7-yl)amino)methyl)piperidin-1-yl)ethyl)piperazin-1-yl)phenyl)amino)piperidine-2,6-dione formate

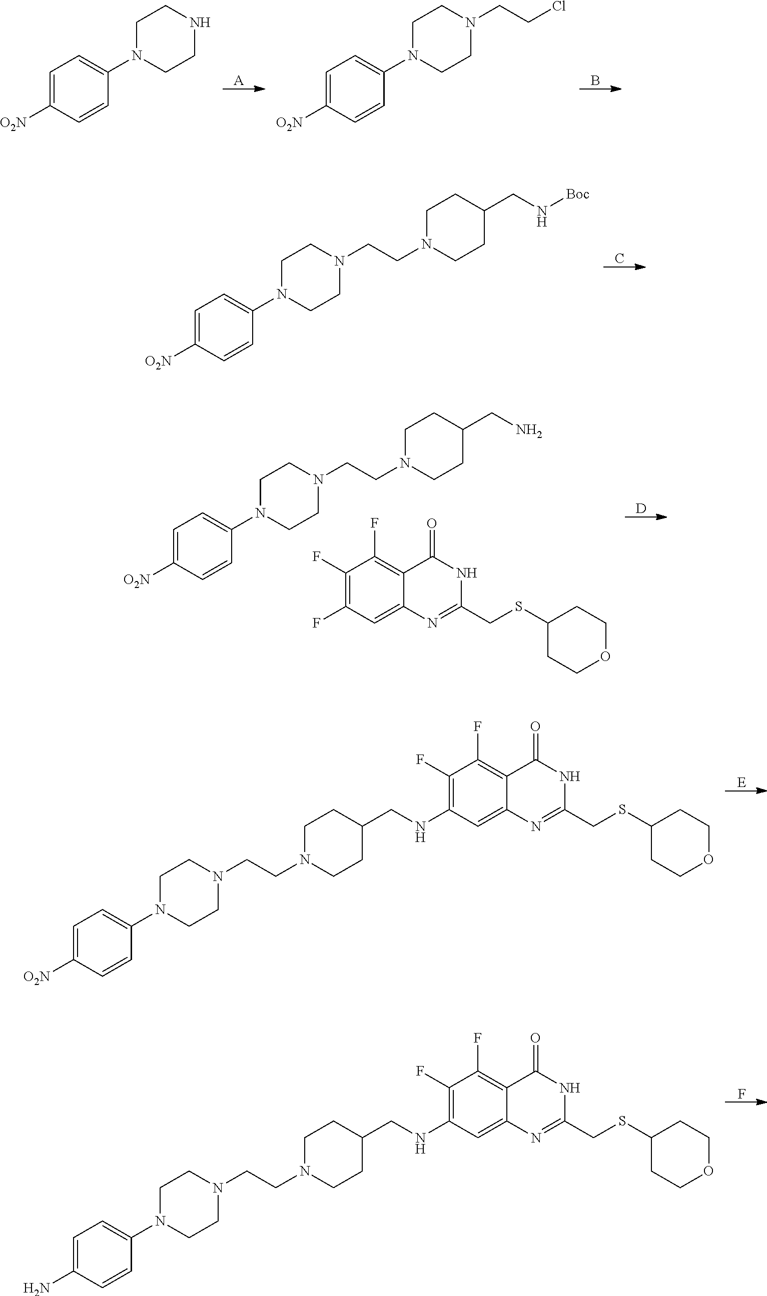

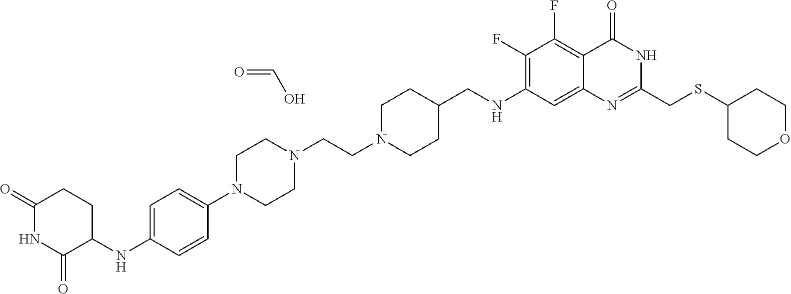 [SMILES: O=C1CCC(Nc2ccc(N3CCN(CCN4CCC(CNc5cc6nc(CSC7CCOCC7)[nH]c(=O)c6c(F)c5F)CC4)CC3)cc2)C(=O)N1 ; O=CO]

### Step A

A solution of 1-(4-nitrophenyl)piperazine (10 g, 48.3 mmol, 1 equiv), 2-chloroacetaldehyde (18.9 g, 241 mmol, 5 equiv) and STAB (20.5 g, 96.5 mmol, 2 equiv) in DCE (50 mL) was stirred for 2 hours. The reaction was concentrated under vacuum and applied onto a silica gel column eluting with DCM/MeOH (90:10) to afford 1-(2-chloroethyl)-4-(4-nitrophenyl)piperazine (11 g, 85%) as a brown oil.

### Step B

A solution of 1-(2-chloroethyl)-4-(4-nitrophenyl)piperazine (11 g, 40.8 mmol, 1 equiv), tert-butyl (piperidin-4-ylmethyl)carbamate (8.74 g, 40.8 mmol, 1 equiv) and DIEA (15.8 g, 122 mmol, 3 equiv) in DMSO (20 mL) was stirred for 3 hours at 80° C. The reaction was quenched with water and extracted with ethyl acetate (3×100 mL). The organics were washed with brine, dried over anhydrous sodium sulfate, filtered and concentrated under vacuum. The crude product was purified by C18 reverse phase chromatography eluting with water/ACN (40:60) to afford tert-butyl ((1-(2-(4-(4-nitrophenyl)piperazin-1-yl)ethyl)piperidin-4-yl)methyl)carbamate (6 g, 33%) as a brown solid. LCMS (ESI, m/z): 448.35 [M+H]⁺.

### Step C

A solution of tert-butyl ((1-(2-(4-(4-nitrophenyl)piperazin-1-yl)ethyl)piperidin-4-yl)methyl)carbamate (6 g, 13.4 mmol, 1 equiv) in HCl in 1,4-dioxane (30 mL, 4M) was stirred for 1 hour. The resulting mixture was concentrated under vacuum to afford (1-(2-(4-(4-nitrophenyl)piperazin-1-yl)ethyl)piperidin-4-yl)methanamine (8.1 g, crude) as a brown solid. The product was used in the next step without further purification.

### Step D

A solution of (1-(2-(4-(4-nitrophenyl)piperazin-1-yl)ethyl)piperidin-4-yl)methanamine (3 g, 8.63 mmol, 1 equiv) 5,6,7-trifluoro-2-(((tetrahydro-2H-pyran-4-yl)thio)methyl)quinazolin-4(3H)-one (2.85 g, 8.63 mmol, 1 equiv) and DIEA (3.35 g, 25.9 mmol, 3 equiv) in NMP (5 mL) was stirred for 8 hours at 80° C. After concentration, the residue was purified by C18 reverse phase chromatography eluting with water/ACN (30:70) to afford 5,6-difluoro-7-(((1-(2-(4-(4-nitrophenyl)piperazin-1-yl)ethyl)piperidin-4-yl)methyl)amino)-2-(((tetrahydro-2H-pyran-4-yl)thio)methyl)quinazolin-4(3H)-one (500 mg, 9%) as a brown solid. LCMS (ESI, m/z): 658.35 [M+H]⁺.

### Step E

A solution of 5,6-difluoro-7-(((1-(2-(4-(4-nitrophenyl)piperazin-1-yl)ethyl)piperidin-4-yl)methyl)amino)-2-(((tetrahydro-2H-pyran-4-yl)thio)methyl)quinazolin-4(3H)-one (500 mg, 0.76 mmol, 1 equiv) B₂(OH)₄ (204 mg, 2.28 mmol, 3 equiv) and 4,4′-bipyridine (12 mg, 0.076 mmol, 0.1 equiv) in DMF (5 mL) was stirred for 10 min. After concentration, the residue was purified by C18 reverse phase chromatography eluting with water/ACN (25:75) to afford 7-(((1-(2-(4-(4-aminophenyl)piperazin-1-yl)ethyl)piperidin-4-yl)methyl)amino)-5,6-difluoro-2-(((tetrahydro-2H-pyran-4-yl)thio)methyl)quinazolin-4(3H)-one (156 mg, 48%) as a brown solid. LCMS (ESI, m/z): 628.40 [M+H]⁺.

### Step F

A solution of 7-(((1-(2-(4-(4-aminophenyl)piperazin-1-yl)ethyl)piperidin-4-yl)methyl)amino)-5,6-difluoro-2-(((tetrahydro-2H-pyran-4-yl)thio)methyl)quinazolin-4(3H)-one (200 mg, 0.32 mmol, 1 equiv) 3-bromopiperidine-2,6-dione (306 mg, 1.60 mmol, 5 equiv) and NaHCO₃ (134 mg, 1.60 mmol, 5 equiv) in ACN (10 mL) was stirred for 8 hours at 90° C. After concentration, the residue was purified by C18 reverse phase chromatography eluting with water/ACN (40:60) and further purified by Prep-HPLC with the following conditions (Column: Xselect CSH OBD Column 30*150 mm, 5 μm; Mobile Phase A: water (0.1% FA), Mobile Phase B: ACN; Flow rate: 60 mL/min; Gradient: 3% B to 15% B in 10 min; Wave Length: 254 nm/220 nm; RT (min): 8.45) to afford 3-((4-(4-(2-(4-(((5,6-difluoro-4-oxo-2-(((tetrahydro-2H-pyran-4-yl)thio)methyl)-3,4-dihydroquinazolin-7-yl)amino)methyl)piperidin-1-yl)ethyl)piperazin-1-yl)phenyl)amino)piperidine-2,6-dione formate (58 mg, 22%) as a grey solid. LCMS (ESI, m/z): 739.35 [M+H]⁺. ¹H NMR (400 MHz, DMSO-d₆) δ 11.90 (s, 1H), 10.75 (s, 1H), 8.20 (s, 1H) HCOOH, 6.92 (s, 1H), 6.79-6.68 (m, 2H), 6.64-6.58 (m, 2H), 6.54 (d, J=7.3 Hz, 1H), 5.36 (s, 1H), 4.25-4.12 (m, 2H), 3.88-3.78 (m, 3H), 3.58 (s, 3H), 3.43-3.35 (m, 2H), 3.28-3.20 (m, 1H), 3.13-2.90 (m, 9H), 2.80-2.70 (m, 1H), 2.62-2.55 (m, 3H), 2.45-2.35 (m, 1H), 2.15-1.98 (m, 3H), 1.92-1.58 (m, 7H), 1.55-1.38 (m, 2H), 1.35-1.15 (m, 2H).

### Example 130: Synthesis of 3-((4-(3-(4-(2-((5-fluoro-4-oxo-2-(((tetrahydro-2H-pyran-4-yl)thio)methyl)-3,4-dihydroquinazolin-7-yl)oxy)ethyl)piperazin-1-yl)azetidin-1-yl)phenyl)amino)piperidine-2,6-dione

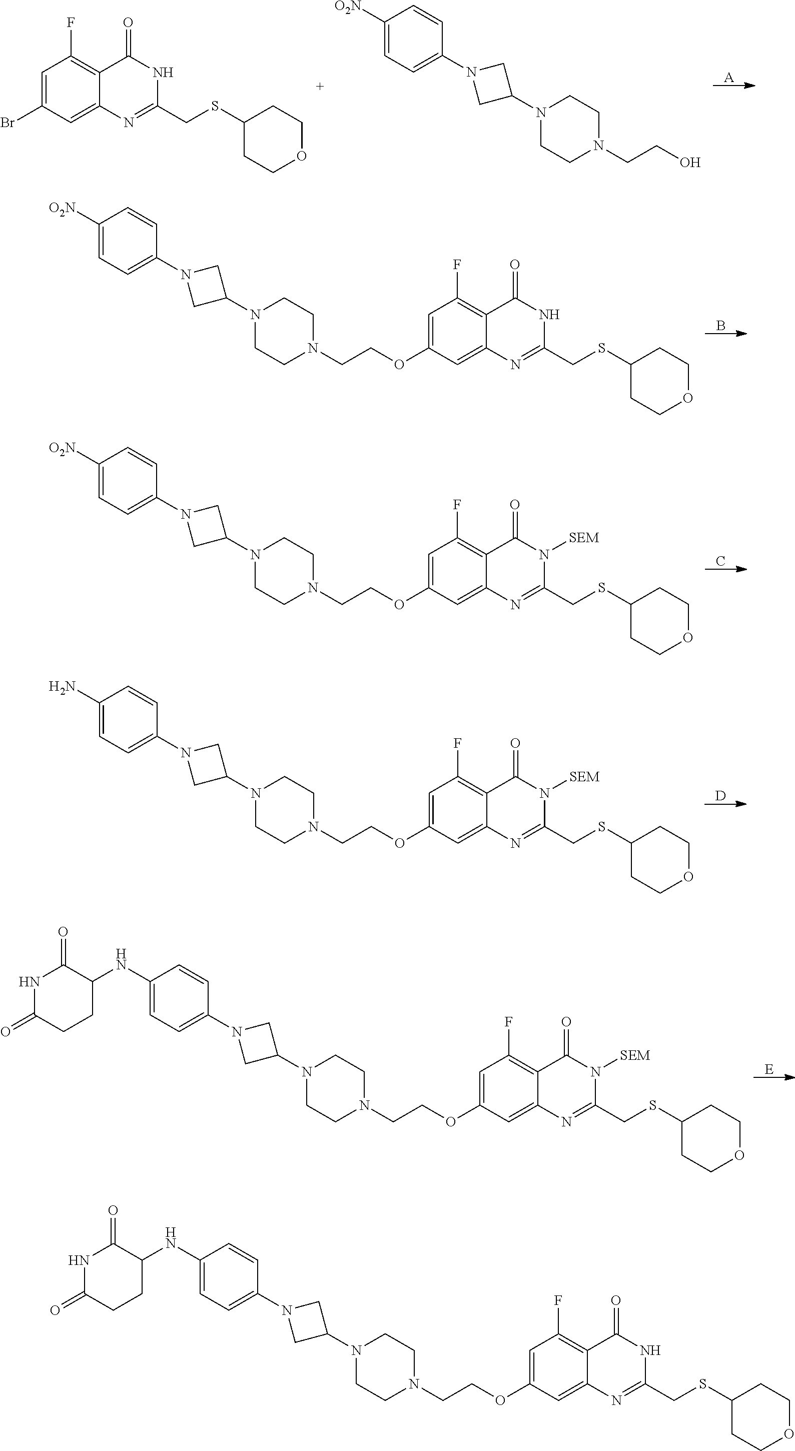

### Step A

A solution of 2-(4-(1-(4-nitrophenyl)azetidin-3-yl)piperazin-1-yl)ethan-1-ol (2.25 g, 7.34 mmol, 1 equiv), 7-bromo-5-fluoro-2-(((tetrahydro-2H-pyran-4-yl)thio)methyl)quinazolin-4(3H)-one (2.74 g, 7.34 mmol, 1 equiv), Cs₂CO₃ (4.79 g, 14.7 mmol, 2 equiv), Pd₂(allyl)₂Cl₂ (54 mg, 0.147 mmol, 0.02 equiv), RockPhos (344 mg, 0.734 mmol, 0.1 equiv) and molecular sieves in toluene (30 mL) was stirred for 1 day at 80° C. under nitrogen atmosphere. The resulting mixture was concentrated under vacuum. The residue was purified by reverse flash chromatography with the following conditions: (column, C18 silica gel; mobile phase, MeCN in water (10 mmol/L NH₄HCO₃), 0% to 44% gradient in 30 min; detector, UV 254 nm). This afforded 5-fluoro-7-(2-(4-(1-(4-nitrophenyl)azetidin-3-yl)piperazin-1-yl)ethoxy)-2-(((tetrahydro-2H-pyran-4-yl)thio)methyl)quinazolin-4(3H)-one (1.25 g, 28%) as a yellow solid. LCMS (ESI, m/z): 599.20 [M+H]⁺.

### Step B

A solution of 5-fluoro-7-(2-(4-(1-(4-nitrophenyl)azetidin-3-yl)piperazin-1-yl)ethoxy)-2-(((tetrahydro-2H-pyran-4-yl)thio)methyl)quinazolin-4(3H)-one (1.2 g, 2.00 mmol, 1 equiv) in THF (30 mL) was treated with NaH (0.10 g, 4.0 mmol, 2 equiv) for 15 min at 0° C. under nitrogen atmosphere followed by the addition of SEM-Cl (0.67 g, 4.01 mmol, 2 equiv) in portions at room temperature. The reaction was then quenched with water and extracted with DCM (3×100 mL). The combined organic layers were washed with brine and dried over anhydrous Na₂SO₄. After filtration, the organic layers were concentrated under vacuum to afford 5-fluoro-7-(2-(4-(1-(4-nitrophenyl)azetidin-3-yl)piperazin-1-yl)ethoxy)-2-(((tetrahydro-2H-pyran-4-yl)thio)methyl)-3-((2-(trimethylsilyl)ethoxy)methyl)quinazolin-4(3H)-one (1 g, 68%) as a crude yellow solid. LCMS (ESI, m/z): 729.30 [M+H]⁺.

### Step C

A solution of 5-fluoro-7-(2-(4-(1-(4-nitrophenyl)azetidin-3-yl)piperazin-1-yl)ethoxy)-2-(((tetrahydro-2H-pyran-4-yl)thio)methyl)-3-((2-(trimethylsilyl)ethoxy)methyl)quinazolin-4(3H)-one (1 g, 1.37 mmol, 1 equiv), Fe (0.38 g, 6.86 mmol, 5 equiv) and NH₄Cl (0.37 g, 6.86 mmol, 5 equiv) in EtOH (2 mL) and water (8 mL) was stirred for 2 hours at 80° C. After concentration, the residue was purified by silica gel column chromatography, eluting with DCM/MeOH (9:1) to afford 7-(2-(4-(1-(4-aminophenyl)azetidin-3-yl)piperazin-1-yl)ethoxy)-5-fluoro-2-(((tetrahydro-2H-pyran-4-yl)thio)methyl)-3-((2-(trimethylsilyl)ethoxy)methyl)quinazolin-4(3H)-one (70 mg, 7%) as a brown yellow oil. LCMS (ESI, m/z): 699.35 [M+H]⁺.

### Step D

A solution of 7-(2-(4-(1-(4-aminophenyl)azetidin-3-yl)piperazin-1-yl)ethoxy)-5-fluoro-2-(((tetrahydro-2H-pyran-4-yl)thio)methyl)-3-((2-(trimethylsilyl)ethoxy)methyl)quinazolin-4(3H)-one (70 mg, 0.10 mmol, 1 equiv), 3-bromopiperidine-2,6-dione (38.5 mg, 0.20 mmol, 2 equiv) and DIEA (38.8 mg, 0.30 mmol, 3 equiv) in NMP (3 mL) was stirred for 2 hours at 80° C. The residue was purified by reverse flash chromatography with the following conditions: (column, C18 silica gel; mobile phase, MeCN in water (10 mmol/L NH₄HCO₃), 0% to 50% gradient in 30 min; detector, UV 254 nm) to afford of 3-((4-(3-(4-(2-((5-fluoro-4-oxo-2-(((tetrahydro-2H-pyran-4-yl)thio)methyl)-3-((2-(trimethylsilyl)ethoxy)methyl)-3,4-dihydroquinazolin-7-yl)oxy)ethyl)piperazin-1-yl)azetidin-1-yl)phenyl)amino)piperidine-2,6-dione (50 mg, 62%) as a brown oil. LCMS (ESI, m/z): 810.45 [M+H]⁺.

### Step E

A solution of 3-((4-(3-(4-(2-((5-fluoro-4-oxo-2-(((tetrahydro-2H-pyran-4-yl)thio)methyl)-3-((2-(trimethylsilyl)ethoxy)methyl)-3,4-dihydroquinazolin-7-yl)oxy)ethyl)piperazin-1-yl)azetidin-1-yl)phenyl)amino)piperidine-2,6-dione (40 mg, 0.049 mmol, 1 equiv) in TFA (2 mL) and DCM (6 mL) was stirred for 1 hour. The resulting mixture was concentrated under vacuum. The crude product was purified by Prep-HPLC with the following conditions (Column: XBridge Prep Phenyl Column, 19*250 mm, 5 μm; Mobile Phase A: water (10 mmol/L NH₄HCO₃), Mobile Phase B: ACN; Flow rate: 25 mL/min; Gradient: 20% B to 40% B in 12 min; Wave Length: 254 nm/220 nm; RT (min): 12.0) to afford 3-((4-(3-(4-(2-((5-fluoro-4-oxo-2-(((tetrahydro-2H-pyran-4-yl)thio)methyl)-3,4-dihydroquinazolin-7-yl)oxy)ethyl)piperazin-1-yl)azetidin-1-yl)phenyl)amino)piperidine-2,6-dione (4.1 mg, 12%) as a grey solid. LCMS (ESI, m/z): 680.30 [M+H]; ¹H NMR (400 MHz, DMSO-d₆) δ 12.15 (s, 1H), 10.74 (s, 1H), 6.99-6.89 (m, 2H), 6.60 (d, J=8.6 Hz, 2H), 6.29 (d, J=8.6 Hz, 2H), 5.19 (d, J=7.1 Hz, 1H), 4.30-4.20 (m, 2H), 4.19-4.10 (m, 1H), 3.89-3.78 (m, 4H), 3.62 (s, 2H), 3.41-3.80 (m, 4H), 3.22-3.00 (m, 2H), 2.75-2.68 (m, 3H), 2.62-2.55 (m, 6H), 2.35-2.28 (m, 3H), 2.15-2.07 (m, 1H), 1.93-1.74 (m, 3H), 1.52-1.38 (m, 2H).

### Example 131: Synthesis of 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl-2,2,3,3,5,5,6,6-d8)phenyl)amino)piperidine-2,6-dione

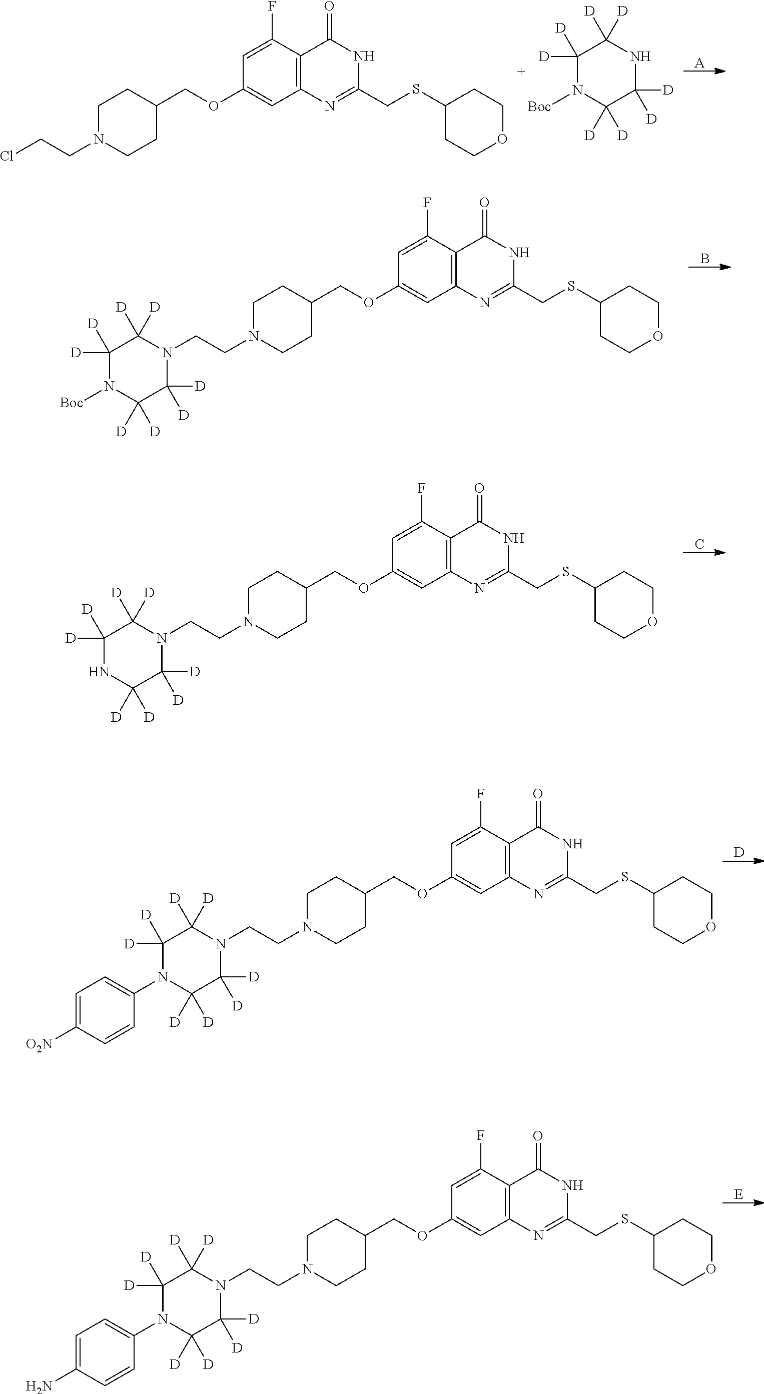

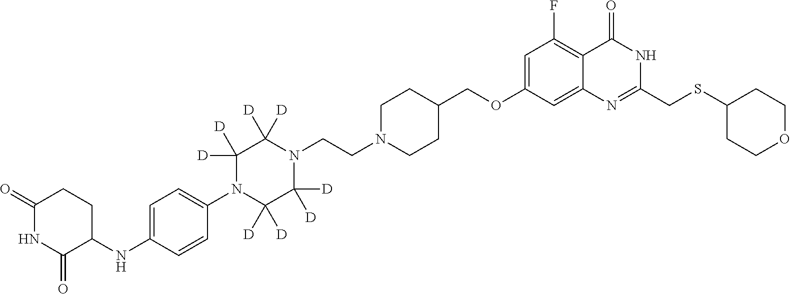 [SMILES: [2H]C1([2H])N(CCN2CCC(COc3cc(F)c4c(=O)[nH]c(CSC5CCOCC5)nc4c3)CC2)C([2H])([2H])C([2H])([2H])N(c2ccc(NC3CCC(=O)NC3=O)cc2)C1([2H])[2H]]

### Step A

A solution of 7-((1-(2-chloroethyl)piperidin-4-yl)methoxy)-5-fluoro-2-(((tetrahydro-2H-pyran-4-yl)thio)methyl)quinazolin-4(3H)-one (4 g, 8.51 mmol, 1 equiv), tert-butyl (2,2,3,3,5,5,6,6-2H8)piperazine-1-carboxylate (6.20 mg, 0.032 mmol, 1.5 equiv) and DIEA (2.20 g, 17.0 mmol, 2 equiv) in DMSO (15 mL) was stirred for 2 hours at 80° C. The residue was purified by silica gel column chromatography, eluting with CH_{C}/MeOH (93:7) to afford tert-butyl 4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazine-1-carboxylate-2,2,3,3,5,5,6,6-d8 (4.1 g, 770%) as a brown solid. LCMS (ESI, m/z): 628.30 [M+H]⁺.

### Step B

A solution of tert-butyl 4-(2-(4-(((5-fluoro-4-oxo-2-(((tetrahydro-2H-pyran-4-yl)thio)methyl)-3,4-dihydroquinazolin-7-yl)oxy)methyl)piperidin-1I-yl)ethyl)piperazine-1I-carboxylate-2,2,3,3,5,5,6,6-d8 (5.78 g, 9.21 mmol, 1 equiv) in HCl in 1,4-dioxane (30 mL, 4 M) was stirred for 2 hours. The resulting mixture was concentrated under reduced pressure to afford crude 5-fluoro-7-((1-(2-(piperazin-1-yl-2,2,3,3,5,5,6,6-d8)ethyl)piperidin-4-yl)methoxy)-2-(((tetrahydro-2H-pyran-4-yl)thio)methyl)quinazolin-4(3H)-one (5.42 g) as a white solid. The crude compound was used in the next step without further purification. LCMS (ESI, m/z): 528.30 [M+H]⁺.

### Step C

A solution of 5-fluoro-7-((1-(2-(piperazin-1-yl-2,2,3,3,5,5,6,6-d8)ethyl)piperidin-4-yl)methoxy)-2-(((tetrahydro-2H-pyran-4-yl)thio)methyl)quinazolin-4(3H)-one (5.42 g, 10.3 mmol, 1 equiv), 4-fluoronitrobenzene (4.35 g, 30.8 mmol, 3 equiv) and DIEA (3.98 g, 30.8 mmol, 3 equiv) in NMP (25 mL) was stirred for 4 hours at 80° C. The mixture was cooled down to room temperature and then quenched with ice water. The precipitated solids were collected by filtration and washed with water. The resulting solid was dried to afford 5-fluoro-7-((1-(2-(4-(4-nitrophenyl)piperazin-1-yl-2,2,3,3,5,5,6,6-d8)ethyl)piperidin-4-yl)methoxy)-2-(((tetrahydro-2H-pyran-4-yl)thio)methyl)quinazolin-4(3H)-one (3.97 g, 60%) as a yellow solid. LCMS (ESI, m/z): 649.30 [M+H]⁺.

### Step D

A solution of 5-fluoro-7-((1-(2-(4-(4-nitrophenyl)piperazin-1-yl-2,2,3,3,5,5,6,6-d8)ethyl)piperidin-4-yl)methoxy)-2-(((tetrahydro-2H-pyran-4-yl)thio)methyl)quinazolin-4(3H)-one (6.4 g, 9.9 mmol, 1 equiv) in EtOH (25 mL) was treated with NH₄Cl (2.11 g, 39.5 mmol, 4 equiv) in water (5 mL) for 5 min followed by the addition of Fe (3.44 mg, 0.060 mmol, 4 equiv) portion-wise. The resulting mixture was stirred for 1 hour at 90° C. The solution was concentrated and the residue was purified by silica gel column chromatography, eluting with DCM/MeOH (91:9) to afford 7-((1-(2-(4-(4-aminophenyl)piperazin-1-yl-2,2,3,3,5,5,6,6-d8)ethyl)piperidin-4-yl)methoxy)-5-fluoro-2-(((tetrahydro-2H-pyran-4-yl)thio)methyl)quinazolin-4(3H)-one (3.02 g, 49%) as a brown solid. LCMS (ESI, m/z): 619.30 [M+H]⁺.

### Step E

A solution of 7-((1-(2-(4-(4-aminophenyl)piperazin-1-yl-2,2,3,3,5,5,6,6-d8)ethyl)piperidin-4-yl)methoxy)-5-fluoro-2-(((tetrahydro-2H-pyran-4-yl)thio)methyl)quinazolin-4(3H)-one (2.5 g, 4.04 mmol, 1 equiv), DIEA (2.61 g, 20.2 mmol, 5 equiv) and 3-bromopiperidine-2,6-dione (2.33 g, 12.1 mmol, 3 equiv) in NMP (20 mL) was stirred overnight at 80° C. The resulting mixture was concentrated under reduced pressure. The residue was purified by silica gel column chromatography, eluting with DCM/EtOH (88:12) to afford crude product. Purification by Prep-HPLC with the following conditions (column: Welch Ultimate AQ-C18, 70*700 mm, 10 μm; Mobile Phase A: water (10 mmol/L NH₄HCO₃), Mobile Phase B: ACN; Flow rate: 200 mL/min; Gradient: 15% B to 40% B in 50 min; Wave Length: 254 nm/220 nm; RT (min):5.0) gave 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl-2,2,3,3,5,5,6,6-d8)phenyl)amino)piperidine-2,6-dione (969 mg, 33%) as an off-white solid. LCMS (ESI, m/z): 730.30 [M+H]⁺; ¹H NMR (300 MHz, DMSO-d₆) δ 12.14 (s, 1H), 10.73 (s, 1H), 6.94-6.82 (m, 2H), 6.74 (d, J=8.8 Hz, 2H), 6.63 (d, J=8.8 Hz, 2H), 5.34 (d, J=7.2 Hz, 1H), 4.26-4.12 (m, 1H), 3.98 (d, J=5.8 Hz, 2H), 3.89-3.76 (m, 2H), 3.62 (s, 2H), 3.40-3.32 (m, 2H), 3.15-2.99 (m, 1H), 2.92 (d, J=10.8 Hz, 2H), 2.82-2.65 (m, 1H), 2.65-2.52 (m, 2H), 2.50-2.41 (m, 4H), 2.16-2.06 (m, 1H), 2.01-1.85 (m, 4H), 1.80-1.69 (m, 3H), 1.53-1.39 (m, 2H), 1.35-1.22 (m, 2H).

Examples 132-133 were synthesized according to the procedure described for the synthesis 5-fluoro-3-(4-methoxybenzyl)-7-((1-(2-(4-nitrophenoxy)ethyl)piperidin-4-yl)methoxy)-2-(((tetrahydro-2H-pyran-4-yl)thio)methyl)quinazolin-4(3H)-one (Example 58) using appropriate building blocks and modified reaction conditions (such as reagents, reagent ratio, temperature, and reaction time) and purification conditions as needed.

| Example | Structure | Analytical Data |
|---|---|---|
| 132 | 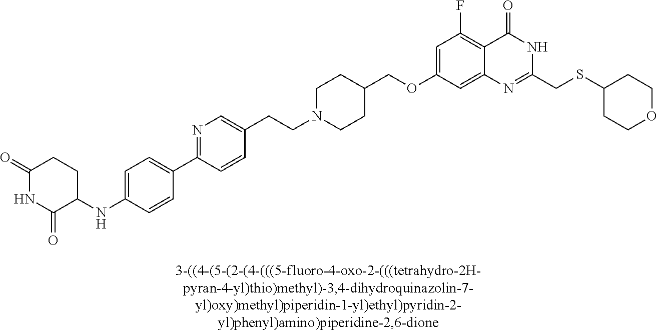 [SMILES: O=C1CCC(Nc2ccc(-c3ccc(CCN4CCC(COc5cc(F)c6c(=O)[nH]c(CSC7CCOCC7)nc6c5)CC4)cn3)cc2)C(=O)N1] | LCMS (ESI, m/z): 715.20 [M + H]⁺. ¹H NMR (300 MHz, DMSO-d₆) δ 12.12 (s, 1H), 10.80 (s, 1H), 8.42 (s, 1H), 7.83 (d, J = 8.8 Hz, 2H), 7.70-7.59 (m, 2H), 6.96-6.91 (m, 2H), 6.76 (d, J = 8.8 Hz, 2H), 6.16 (d, 8.0 Hz, , 1H), 4.51- 4.42 (m, 1H), 3.98 (s, 2H), 3.82 (d, J = 6.0 Hz, 2H), 3.63 (s, 2H), 3.08-2.98 (m, 3H), 2.84-2.70 (m, 3H), 2.64-2.51 (m, 4H), 2.17-2.01 (m, 1H), 2.06-1.84 (m, 5H), 1.76 (d, J = |
| | | 11.5 Hz, 3H), 1.46- |
| | | 1.33 (m, 4H) |
| 133 | 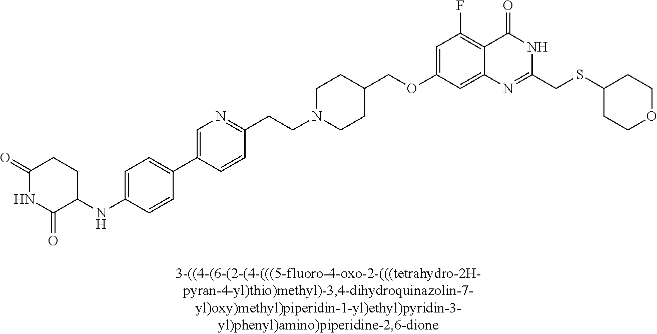 [SMILES: O=C1CCC(Nc2ccc(-c3ccc(CCN4CCC(COc5cc(F)c6c(=O)[nH]c(CSC7CCOCC7)nc6c5)CC4)nc3)cc2)C(=O)N1] | LCMS (ESI, m/z): 715.20 [M + H]⁺; ¹H NMR (400 MHz, DMSO-d₆) δ 12.10 (s, 1H), 10.81 (s, 1H), 8.69 (d, J = 2.4 Hz, 1H), 7.85 (dd, J = 8.1, 2.5 Hz, 1H), 7.46 (d, J = 8.3 Hz, 2H), 7.29 (d, J = 8.1 Hz, 1H), 7.01-6.62 (m, 4H), 6.09 (d, J = 7.8 Hz, 1H), 4.55- 4.25 (m, 1H), 3.98 (d, J = 5.8 Hz, 2H), 3.87-3.79 (m, 2H), 3.62 (s, 2H), 3.42- 3.39 (m, 1H), 3.09- 3.04 (m, 1H), 3.03- 2.95 (m, 2H), 2.92- 2.85 (m, 2H), 2.84- |
| | | 2.75 (m, 1H), 2.72- |
| | | 2.62 (m, 3H), 2.23- |
| | | 2.09 (m, 1H), 2.06- |
| | | 1.84 (m, 5H), 1.79- |
| | | 1.65 (m, 3H), 1.56- |
| | | 1.40 (m, 2H), 1.39- |
| | | 1.23 (m, 3H), 0.90- |
| | | 0.82 (m, 1H). |

### Example 134: Synthesis of N-(2,6-dioxopiperidin-3-yl)-3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1l-yl)ethyl)piperazin-1-yl)bicyclo[1.1.1]pentane-1l-carboxamide

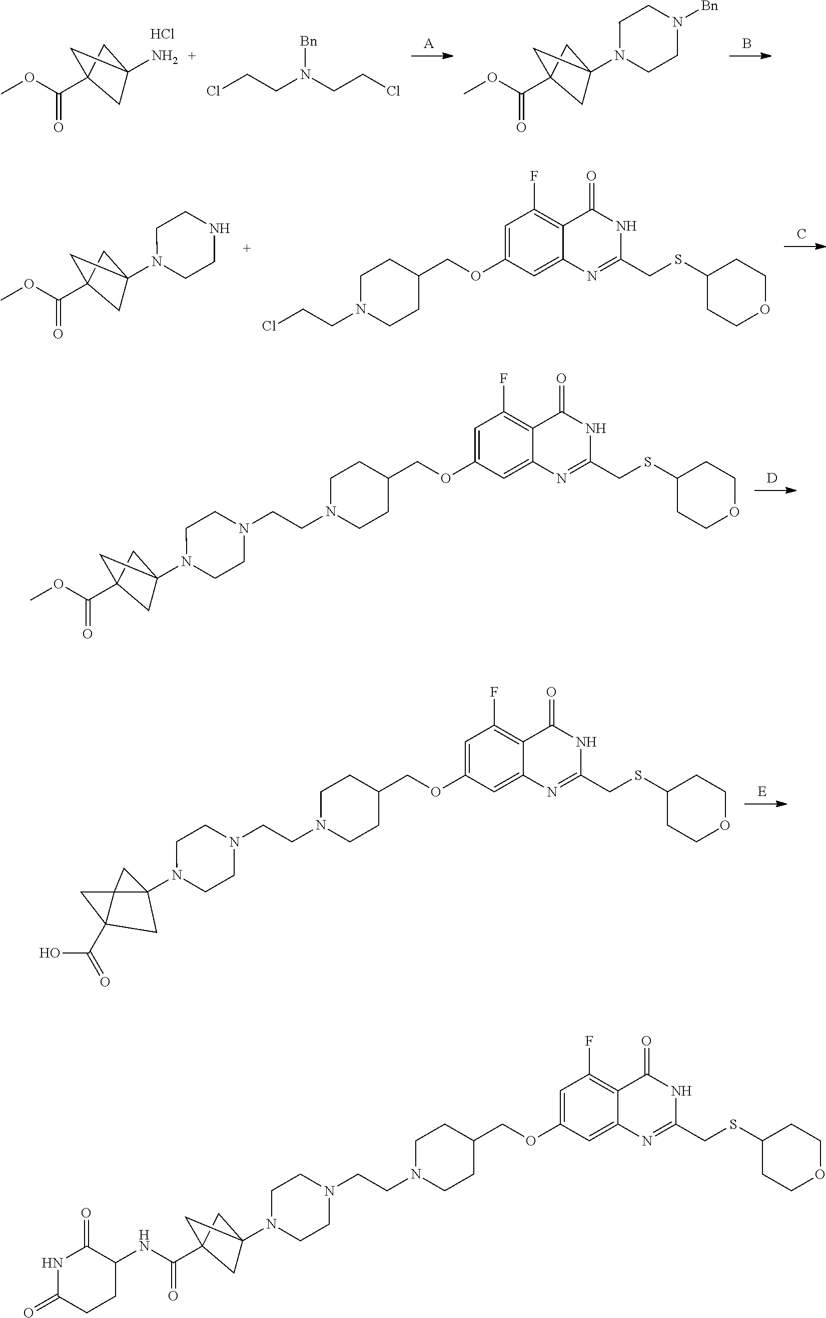

### Step A

To a stirred mixture of methyl 3-aminobicyclo[1.1.1]pentane-1-carboxylate hydrochloride (500 mg, 2.82 mmol, 1 equiv), K₂CO₃ (1.17 g, 8.45 mmol, 3 equiv) and KI (234 mg, 1.41 mmol, 0.5 equiv) in ACN (9 mL) was added N-benzyl-2-chloro-N-(2-chloroethyl)ethan-1-amine (756 mg, 2.82 mmol, 1 equiv) portion-wise under a nitrogen atmosphere. The final reaction mixture was irradiated with microwave radiation for 5 hours at 80° C. The resulting mixture was filtered and the filter cake was washed with DCM (5×100 mL). The filtrate was concentrated under reduced pressure. The residue was purified by reverse-phase flash chromatography with the following conditions: column, C18 silica gel; mobile phase, ACN in water (0.1% FA), 0% to 50% gradient in 20 min; detector, UV 254 nm to give crude product that was further purified by reverse-phase flash chromatography with the following conditions: column, C18 silica gel; mobile phase, ACN in water (10 mmol/L NH₄HCO₃), 3% to 80% gradient in 20 min; detector, UV 254 nm to give methyl 3-(4-benzylpiperazin-1-yl)bicyclo[1.1.1]pentane-1-carboxylate (580 mg, 69%) as a light yellow solid. LCMS (ESI, m/z): 301.20 [M+H]⁺.

### Step B

A mixture of methyl 3-(4-benzylpiperazin-1-yl)bicyclo[1.1.1]pentane-1-carboxylate (1.75 g, 5.84 mmol, 1 equiv) and Pd/C (2 g, 18.8 mmol, 3.2 equiv) in MeOH (100 mL) was stirred for 3 hours under hydrogen atmosphere. The resulting mixture was filtered and the filter cake was washed with MeOH (5×100 mL). The filtrate was concentrated under reduced pressure to afford methyl 3-(piperazin-1-yl)bicyclo[1.1.1]pentane-1-carboxylate (1.18 g) as a white solid. The crude product was used in the next step directly without further purification. LCMS (ESI, m/z): 211.15 [M+H]⁺.

### Step C

A mixture of methyl 3-(piperazin-1-yl)bicyclo[1.1.1]pentane-1-carboxylate (450 mg, 2.14 mmol, 1 equiv), 7-((1-(2-chloroethyl)piperidin-4-yl)methoxy)-5-fluoro-2-(((tetrahydro-2H-pyran-4-yl)thio)methyl)quinazolin-4(3H)-one (1.21 g, 2.57 mmol, 1.2 equiv) and DIEA (830 mg, 6.42 mmol, 3 equiv) in DMSO (9 mL) was set up twice in two vessels. The reaction mixtures in both vessels were stirred for 3.5 hours at 80° C. under nitrogen atmosphere. The reaction mixtures were combined and purified directly by reverse-phase flash chromatography with the following conditions: column, C18 silica gel; mobile phase, ACN in water (0.1% FA), 0% to 50% gradient in 30 min; detector, UV 254 nm to give methyl 3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)bicyclo[1.1.1]pentane-1-carboxylate (3.06 g) as an orange oil. LCMS (ESI, m/z): 644.50 [M+H]⁺.

### Step D

A mixture of methyl 3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)bicyclo[1.1.1]pentane-1-carboxylate (3.06 g, 4.76 mmol, 1 equiv) and NaOH (761 mg, 19.4 mmol, 4.02 equiv) in water (40 mL) was stirred for 2 hours. The residue was purified by reverse-phase flash chromatography with the following conditions: column, C18 silica gel; mobile phase, ACN in water (10 mmol/L NH₄HCO₃), 10% to 50% gradient in 10 min; detector, UV 254 nm to give 3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)bicyclo[1.1.1]pentane-1-carboxylic acid (1.6 g, 28%) as a white solid. LCMS (ESI, m/z): 630.50 [M+H]⁺.

### Step E

To a stirred mixture of 3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)bicyclo[1.1.1]pentane-1-carboxylic acid (200 mg, 0.318 mmol, 1 equiv), 3-aminopiperidine-2,6-dione hydrochloride (105 mg, 0.636 mmol, 2 equiv) and DIEA (185 mg, 1.43 mmol, 4.5 equiv) in DMF (10 mL) was added HATU (242 mg, 0.636 mmol, 2 equiv) in portions. The resulting mixture was stirred for 3 hours. The solution was concentrated and the residue was purified by silica gel column chromatography, eluting with DCM/EtOH (4:1) to afford N-(2,6-dioxopiperidin-3-yl)-3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)bicyclo[1.1.1]pentane-1-carboxamide (104 mg, 41%) as a white solid. LCMS (ESI, m/z): 740.25 [M+H]⁺. ¹H NMR (400 MHz, DMSO-d₆) δ 12.10 (br, 1H), 10.80 (s, 1H), 8.02 (d, J=8.4 Hz, 1H), 6.89-6.79 (m, 2H), 4.55-4.47 (m, 1H), 3.98 (d, J=5.8 Hz, 2H), 3.87-3.79 (m, 2H), 3.62 (s, 2H), 3.35-3.25 (m, 3H), 3.09-3.04 (m, 1H), 2.92-2.85 (m, 2H), 2.84-2.68 (m, 1H), 2.65-2.50 (m, 11H), 2.06-1.84 (m, 14H), 1.80-1.75 (m, 3H), 1.56-1.40 (m, 2H), 1.39-1.23 (m, 3H).

### Example 135: Synthesis of 3-((3-fluoro-4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)phenyl)amino)piperidine-2,6-dione formate

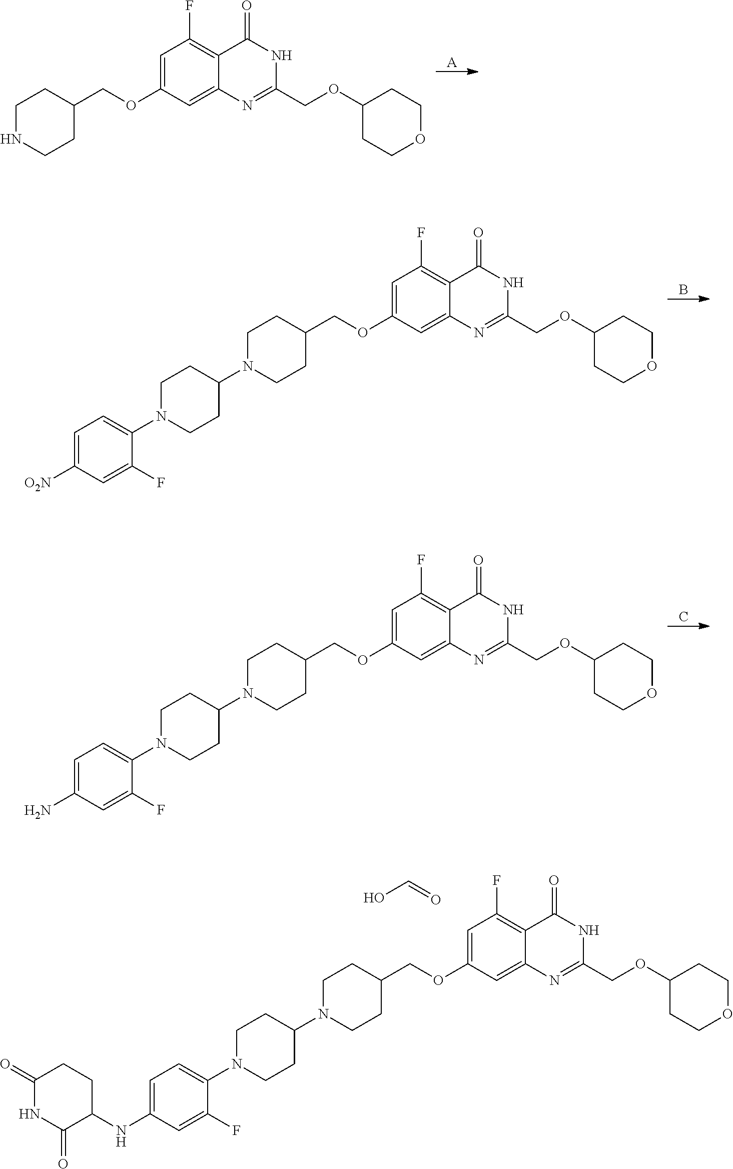

### Step A

A solution of 5-fluoro-7-(piperidin-4-ylmethoxy)-2-(((tetrahydro-2H-pyran-4-yl)oxy)methyl)quinazolin-4(3H)-one (2.1 g, 5.4 mmol, 1 equiv), 1-(2-fluoro-4-nitrophenyl)piperidin-4-one (3.83 g, 16.1 mmol, 3 equiv) and STAB (3.41 g, 16.1 mmol, 3 equiv) in DCE (100 mL) was stirred for 2 days. The solution was concentrated and the residue was purified by silica gel column chromatography, eluting with DCM/MeOH (86:14) to afford 5-fluoro-7-((1′-(2-fluoro-4-nitrophenyl)-[1,4′-bipiperidin]-4-yl)methoxy)-2-(((tetrahydro-2H-pyran-4-yl)oxy)methyl)quinazolin-4(3H)-one (1.8 g, 55%) as a yellow solid. LCMS (ESI, m/z): 614.30 [M+H]⁺.

### Step B

A solution of 5-fluoro-7-((1′-(2-fluoro-4-nitrophenyl)-[1,4′-bipiperidin]-4-yl)methoxy)-2-(((tetrahydro-2H-pyran-4-yl)oxy)methyl)quinazolin-4(3H)-one (1.8 g, 2.93 mmol, 1 equiv) in DMF (10 mL) was treated with B₂(OH)₄ (0.79 g, 8.80 mmol, 3 equiv) for 2 min followed by the addition of 4,4′-bipyridine (0.05 g, 0.29 mmol, 0.1 equiv) portion-wise. The solution was stirred for 10 min and then the solution was concentrated and the residue was purified by reverse-phase flash chromatography with the following conditions: column, C18 silica gel; mobile phase, ACN in water (10 mmol/L NH₄HCO₃), 0% to 70% gradient in 30 min; detector, UV 254 nm. This resulted in 7-((1′-(4-amino-2-fluorophenyl)-[1,4′-bipiperidin]-4-yl)methoxy)-5-fluoro-2-(((tetrahydro-2H-pyran-4-yl)oxy)methyl)quinazolin-4(3H)-one (1.3 g, 76%) as a grey solid. LCMS (ESI, m/z): 584.35 [M+H]⁺.

### Step C

A solution of 7-((1′-(4-amino-2-fluorophenyl)-[1,4′-bipiperidin]-4-yl)methoxy)-5-fluoro-2-(((tetrahydro-2H-pyran-4-yl)oxy)methyl)quinazolin-4(3H)-one (1.2 g, 2.1 mmol, 1 equiv), 3-bromopiperidine-2,6-dione (1.18 g, 6.17 mmol, 3 equiv) and NaHCO₃ (0.86 g, 10.3 mmol, 5 equiv) in ACN (100 mL) was stirred for 3 days at 90° C. The resulting mixture was concentrated under vacuum. The residue was purified by silica gel column chromatography, eluting with DCM/EtOH (10:1) to afford the crude product. The product was repurified by reverse-phase flash chromatography with the following conditions: column, C18 silica gel; mobile phase, ACN in water (0.1% FA), 0% to 50% gradient in 30 min; detector, UV 254 nm to give 3-((3-fluoro-4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)phenyl)amino)piperidine-2,6-dione formate (411 mg, 29%) as a white solid. LCMS (ESI, m/z): 695.30 [M+H]⁺; ¹H NMR (400 MHz, DMSO-d₆) δ 12.04 (s, 1H), 10.78 (s, 1H), 8.18 (s, 1H), 7.05-6.74 (m, 3H), 6.51 (dd, J=14.9, 2.6 Hz, 1H), 6.42 (dd, J=8.7, 2.6 Hz, 1H), 5.81 (d, J=7.7 Hz, 1H), 4.38 (s, 2H), 4.31-4.20 (m, 1H), 4.00 (d, J=5.7 Hz, 2H), 3.87-3.79 (m, 2H), 3.71-3.60 (m, 1H), 3.36 (d, J=2.7 Hz, 1H), 3.31 (d, J=2.7 Hz, 1H), 3.19 (d, J=10.8 Hz, 2H), 3.09 (d, J=10.8 Hz, 2H), 2.78-2.67 (m, 1H), 2.66-2.52 (m, 5H), 2.43-2.35 (m, 2H), 2.13-2.03 (m, 1H), 1.90-1.78 (m, 7H), 1.70-1.58 (m, 2H), 1.54-1.33 (m, 4H).

### Example 136: Synthesis of 3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione

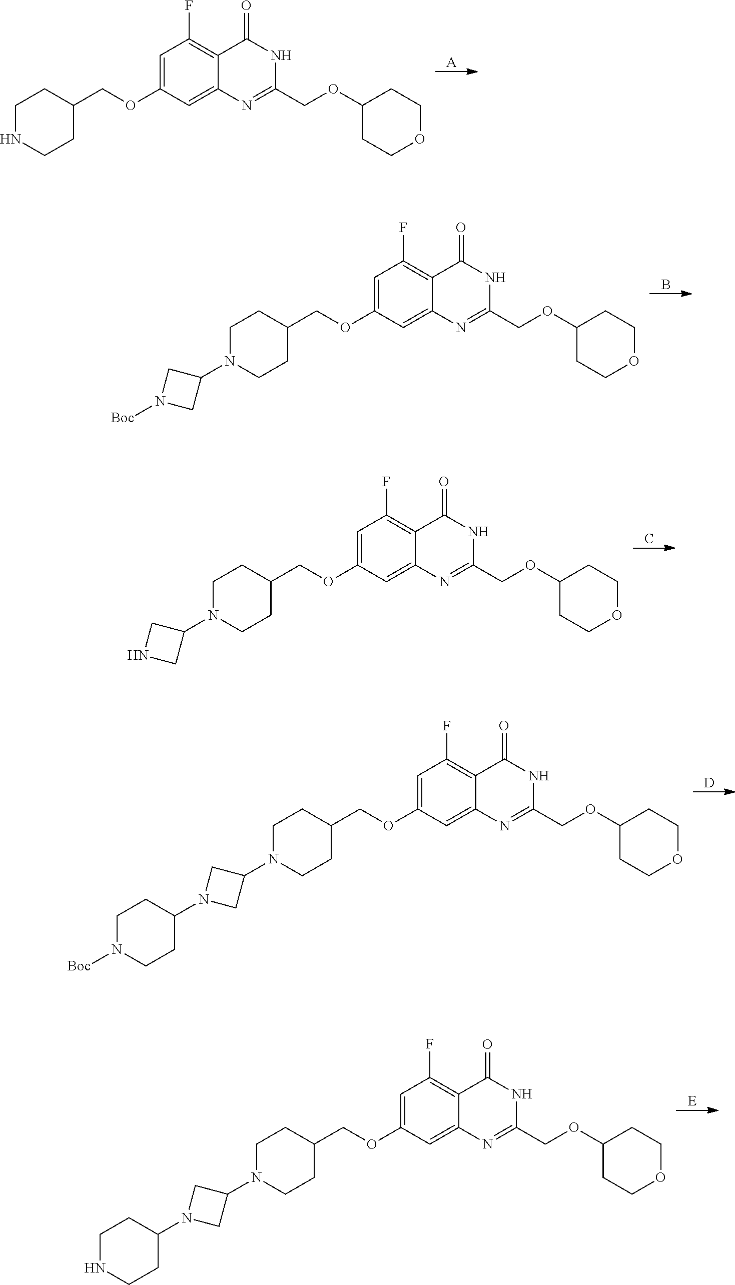

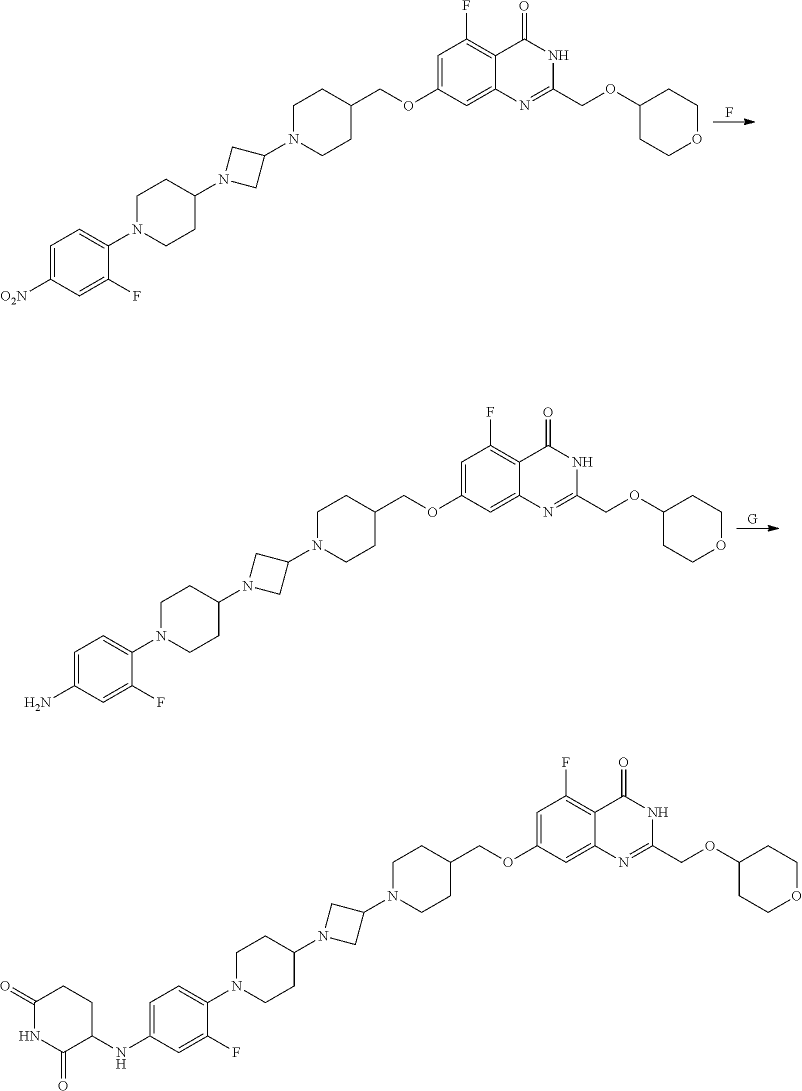

### Step A

A solution of 5-fluoro-7-(piperidin-4-ylmethoxy)-2-(((tetrahydro-2H-pyran-4-yl)oxy)methyl)quinazolin-4(3H)-one (4.2 g, 10.7 mmol, 1 equiv) in DCE (50 mL) was treated with tert-butyl 3-oxoazetidine-1-carboxylate (5.51 g, 32.2 mmol, 3 equiv) for 1 hour followed by the addition of STAB (11.4 g, 53.7 mmol, 5 equiv) in portions. The resulting mixture was stirred for 2 hours then the solution was concentrated under vacuum and applied onto a silica gel column eluting with DCM/MeOH (90:10) to afford tert-butyl 3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidine-1-carboxylate (4.1 g, 70%) as a brown solid. LCMS (ESI, m/z): 547.40 [M+H]⁺.

### Step B

A solution of tert-butyl 3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidine-1-carboxylate (4.1 g, 7.50 mmol, 1 equiv) in DCM (80 mL) was treated with TFA (20 mL) for 1 hour. The resulting mixture was concentrated under vacuum. The pH value of the solution was adjusted to 7 with NaHCO₃. The solution was concentrated under vacuum and applied onto a silica gel column eluting with DCM/MeOH (80:20) to afford 7-((1-(azetidin-3-yl)piperidin-4-yl)methoxy)-5-fluoro-2-(((tetrahydro-2H-pyran-4-yl)oxy)methyl)quinazolin-4(3H)-one (3 g, 90%) as a brown oil. LCMS (ESI, m/z): 447.40 [M+H]⁺.

### Step C

A solution of 7-((1-(azetidin-3-yl)piperidin-4-yl)methoxy)-5-fluoro-2-(((tetrahydro-2H-pyran-4-yl)oxy)methyl)quinazolin-4(3H)-one (3 g, 6.72 mmol, 1 equiv), tert-butyl 4-oxopiperidine-1-carboxylate (6.69 g, 33.6 mmol, 5 equiv) and STAB (4.27 g, 20.2 mmol, 3 equiv) in DCE (50 mL) was stirred for 4 hours. The solution was concentrated under vacuum and applied onto a silica gel column with DCM/MeOH (90:10) to afford tert-butyl 4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidine-1-carboxylate (2.8 g, 66%) as a brown solid. LCMS (ESI, m/z): 630.45 [M+H]⁺.

### Step D

A solution of tert-butyl 4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidine-1-carboxylate (2.8 g, 4.5 mmol, 1 equiv) in HCl in 1,4-dioxane (4M, 50 mL) was stirred for 2 hours. The resulting mixture was concentrated under vacuum to afford 5-fluoro-7-((1-(1-(piperidin-4-yl)azetidin-3-yl)piperidin-4-yl)methoxy)-2-(((tetrahydro-2H-pyran-4-yl)oxy)methyl)quinazolin-4(3H)-one (3 g, crude) as a brown solid. The product was used in the next step without further purification. LCMS (ESI, m/z): 530.35 [M+H]⁺.

### Step E

A solution of 5-fluoro-7-((1-(1-(piperidin-4-yl)azetidin-3-yl)piperidin-4-yl)methoxy)-2-(((tetrahydro-2H-pyran-4-yl)oxy)methyl)quinazolin-4(3H)-one (2.3 g, 4.34 mmol, 1 equiv), 1,2-difluoro-4-nitrobenzene (0.62 g, 3.91 mmol, 0.9 equiv) and TEA (1.32 g, 13.0 mmol, 3 equiv) in DMF (5 mL) was stirred for 4 hours at 80° C. After concentration, the residue was purified by C18 reverse phase chromatography eluting with water/ACN (60:40) to afford 5-fluoro-7-((1-(1-(1-(2-fluoro-4-nitrophenyl)piperidin-4-yl)azetidin-3-yl)piperidin-4-yl)methoxy)-2-(((tetrahydro-2H-pyran-4-yl)oxy)methyl)quinazolin-4(3H)-one (2 g, 69%) as a brown solid. LCMS (ESI, m/z): 669.35 [M+H]⁺.

### Step F

A solution of 5-fluoro-7-((1-(1-(1-(2-fluoro-4-nitrophenyl)piperidin-4-yl)azetidin-3-yl)piperidin-4-yl)methoxy)-2-(((tetrahydro-2H-pyran-4-yl)oxy)methyl)quinazolin-4(3H)-one (1.5 g, 2.2 mmol, 1 equiv), B₂(OH)₄ (0.60 g, 6.73 mmol, 3 equiv) and 4,4′-bipyridine (0.07 g, 0.449 mmol, 0.2 equiv) in DMF (5 mL) was stirred for 10 min. After concentration, the residue was purified by C18 reverse phase chromatography eluting with water/ACN (60:40) to afford 7-((1-(1-(1-(4-amino-2-fluorophenyl)piperidin-4-yl)azetidin-3-yl)piperidin-4-yl)methoxy)-5-fluoro-2-(((tetrahydro-2H-pyran-4-yl)oxy)methyl)quinazolin-4(3H)-one (550 mg, 38%) as a brown solid. LCMS (ESI, m/z): 639.40 [M+H]⁺.

### Step G

A solution of 7-((1-(1-(1-(4-amino-2-fluorophenyl)piperidin-4-yl)azetidin-3-yl)piperidin-4-yl)methoxy)-5-fluoro-2-(((tetrahydro-2H-pyran-4-yl)oxy)methyl)quinazolin-4(3H)-one (550 mg, 0.861 mmol, 1 equiv), 3-bromopiperidine-2,6-dione (827 mg, 4.31 mmol, 5 equiv) and NaHCO₃ (362 mg, 4.31 mmol, 5 equiv) in ACN (20 mL) was stirred for 3 days at 90° C. The solution was concentrated under vacuum and applied onto a silica gel column eluting with DCM/EtOH (84:16) to afford 3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione (145 mg, 23%) as a white solid. LCMS (ESI, m/z): 750.35 [M+H]⁺. ¹H NMR (400 MHz, DMSO-d₆) δ 12.04 (s, 1H), 10.77 (s, 1H), 10.28 (s, 1H), 6.97-6.80 (m, 3H), 6.61-6.44 (m, 1H), 6.44 (d, J=8.8 Hz, 1H), 5.85 (d, J=7.7 Hz, 1H), 4.38 (s, 2H), 4.30-4.21 (m, 1H), 4.18-3.92 (m, 4H), 3.88-3.78 (m, 2H), 3.70-3.61 (m, 1H), 3.22-3.09 (m, 4H), 2.88-2.68 (m, 3H), 2.65-2.55 (m, 4H), 2.08 (s, 3H), 1.99-1.72 (m, 10H), 1.59-1.25 (m, 7H).

Examples 136a and 136b: Separation of (S)-3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione and (R)-3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione

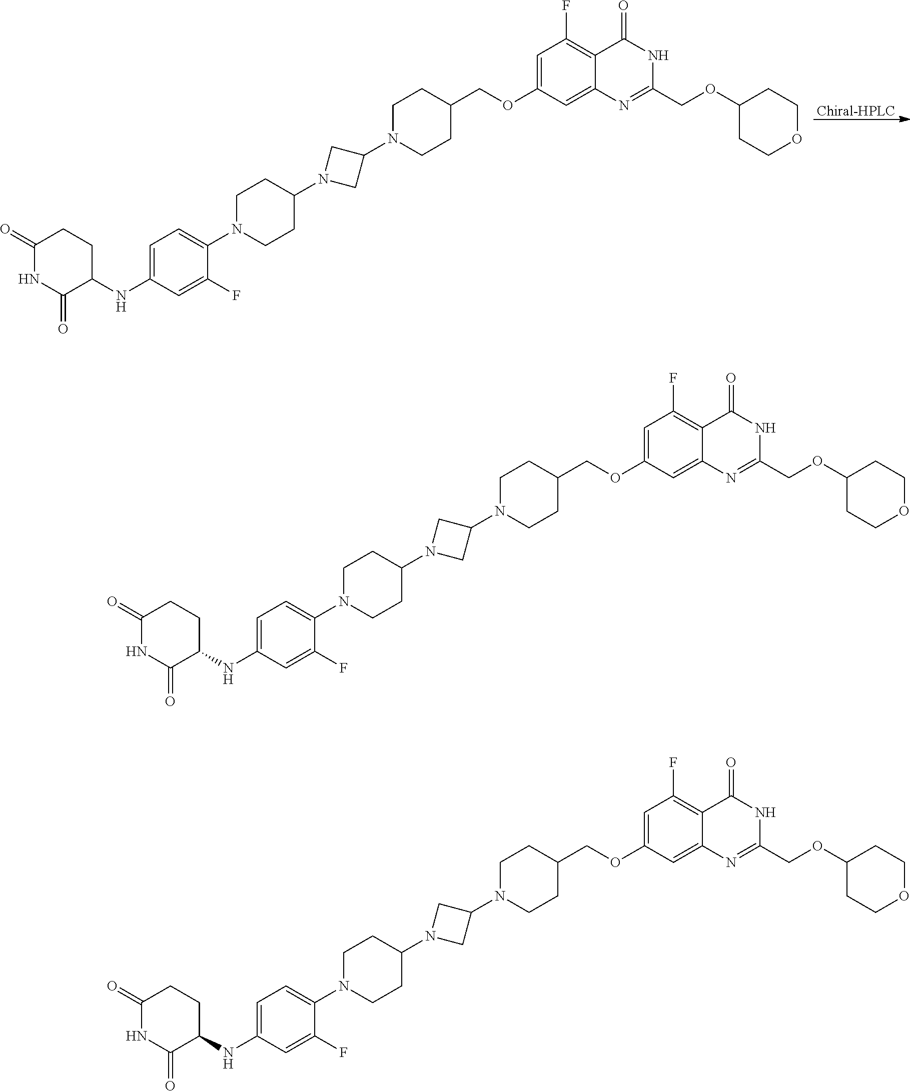

40 mg of the racemic 3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione (Example 136) was separated by Chiral-Prep-HPLC with the following conditions: (Column: CHIRALPAK IF-3, 2*25 cm, 5 μm; Mobile Phase A: Hex (0.5% 2 M NH₃-MeOH)-HPLC, Mobile Phase B: EtOH:DCM=1:1-HPLC; Flow rate: 20 mL/min; Gradient: isocratic 80% B; Wavelength: 220/254 nm; RT (min): 7.52 (Example 136a); RT (min): 13.02 (Example 136b); Sample Solvent: MeOH:DCM=1:1-HPLC; Injection Volume: 1.0 mL; Number of runs: 2). The fractions were directly concentrated and then lyophilized to afford the title compounds. Absolute stereochemistry was not assigned.

Example 136a: The first eluting isomer isolated using the above conditions (6.9 mg) as a white solid. LCMS (ESI, m/z): 750.30 [M+H]⁺. 1H NMR (400 MHz, DMSO-d₆) δ 12.04 (s, 1H), 10.78 (s, 1H), 7.01-6.76 (m, 3H), 6.55 (dd, J=14.9, 2.5 Hz, 1H), 6.41 (dd, J=8.5, 2.6 Hz, 1H), 5.82 (d, J=7.7 Hz, 1H), 4.39 (s, 2H), 4.33-4.22 (m, 1H), 4.01 (d, J=4.0 Hz, 2H), 3.86-3.79 (m, 2H), 3.70-3.63 (m, 1H), 3.40-3.35 (m, 7H), 3.18-3.10 (m, 2H), 2.90-2.66 (m, 4H), 2.66-2.54 (m, 3H), 2.12-2.02 (m, 1H), 2.00-1.70 (m, 10H), 1.55-1.23 (m, 6H). Column: CHIRALPAK IF-3; Mobile Phase A: Hex (0.2% DEA):(EtOH:DCM=1:1)=20:80; Flow rate: 1.0 mL/min; Retention time: 1.61 min (faster peak).

Example 136b: The second eluting isomer isolated using the above conditions (1.7 mg) as a white solid. LCMS (ESI, m/z): 750.35 [M+H]⁺. 1H NMR (400 MHz, DMSO-d₆) δ 12.34-11.30 (m, 2H), 10.79 (s, 1H), 7.15-6.74 (m, 3H), 6.55 (dd, J=14.9, 2.5 Hz, 1H), 6.41 (dd, J=8.5, 2.6 Hz, 1H), 4.93-4.59 (m, 2H), 4.40 (s, 4H), 4.30-4.20 (m, 2H), 4.09-4.01 (m, 2H), 3.88-3.79 (m, 2H), 3.73-3.62 (m, 2H), 3.34-3.30 (m, 3H), 3.28-3.20 (m, 2H), 3.00-2.85 (m, 1H), 2.80-2.53 (m, 6H), 2.20-1.80 (m, 9H), 1.73-1.40 (m, 5H), 1.30-1.20 (m, 1H). Column: CHIRALPAK IF-3; Mobile Phase A: Hex (0.2% DEA):(EtOH:DCM=1:1)=20:80; Flow rate: 1.0 mL/min; Retention time: 2.69 min (slower peak).

Examples 137-139 were synthesized according to the procedure described for steps E-G of the synthesis 3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione (Example 136; steps E-G) using appropriate building blocks and modified reaction conditions (such as reagents, reagent ratio, temperature, and reaction time) and purification conditions as needed.

| Example | Structure | Analytical Data |
|---|---|---|
| 137 | 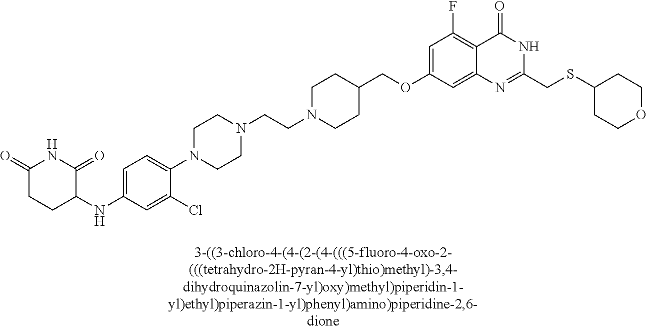 [SMILES: O=C1CCC(Nc2ccc(N3CCN(CCN4CCC(COc5cc(F)c6c(=O)[nH]c(CSC7CCOCC7)nc6c5)CC4)CC3)c(Cl)c2)C(=O)N1] | LCMS (ESI, m/z): 756.20 [M + H]⁺; ¹H-NMR (400 MHz, DMSO-d₆) δ 12.15 (s, 1H), 10.75 (s, 1H), 6.98 (d, 8.0 Hz, 1H), 6.90- 6.85 (m, 2H), 6.76 (s, 1H), 6.68-6.59 (m, 1H), 5.86 (d, 8.0 Hz, 1H), 4.35-4.24 (m, 1H), 3.98 (d, 6.4 Hz, 2H), 3.82-3.73 (m, 2H), 3.62 (s, 2H), 3.38- 3.31 (m, 3H), 3.12- 3.01 (m, 1H), 2.95- 2.85 (m, 2H), 2.84- 2.80 (m, 2H), 2.79-2.68 (m, 2H), 2.55 (s, 3H), 2.49-2.38 (m, 3H), 2.15- 2.05 (m, 1H), 2.02- |
| | | 1.80 (m, 5H), 1.79-1.70 |
| | | (m, 3H), 1.52-1.40 (m, |
| | | 2H), 1.35-1.20 (m, 3H) |
| 138 | 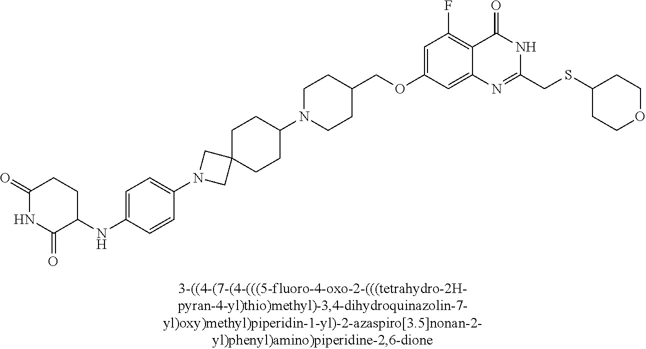 [SMILES: O=C1CCC(Nc2ccc(N3CC4(CCC(N5CCC(COc6cc(F)c7c(=O)[nH]c(CSC8CCOCC8)nc7c6)CC5)CC4)C3)cc2)C(=O)N1] | LCMS (ESI, m/z): 733.30 [M + H]⁺; ¹H NMR (400 MHz, DMSO-d₆) δ 12.15 (s, 1H), 10.73 (s, 1H), 8.23 (s, 1H) HCOOH, 6.93-6.82 (m, 2H), 6.60 (d, J = 8.8 Hz, 2H), 6.27 (d, J = 8.8 Hz, 2H), 5.14 (br, 1H), 4.20-4.11 (m, 1H), 3.99-3.95 (m, 1H), 3.84-3.78 (m, 2H), 3.62 (s, 1H), 3.41- 3.40 (m, 1H), 3.39- 3.37 (m, 3H), 3.09- 3.03 (m, 1H), 2.90- 2.83 (m, 2H), 2.73- 2.65 (m, 1H), 2.56- 2.54 (m, 6H), 2.35- 2.26 (m, 1H), 2.25- 2.16 (m, 2H), 2.15- |
| | | 2.06 (m, 1H), 1.96- |
| | | 1.85 (m, 4H), 1.80- |
| | | 1.65 (m, 6H), 1.49- |
| | | 1.42 (m, 4H), 1.36- |
| | | 1.25 (m, 3H). |
| 139 | 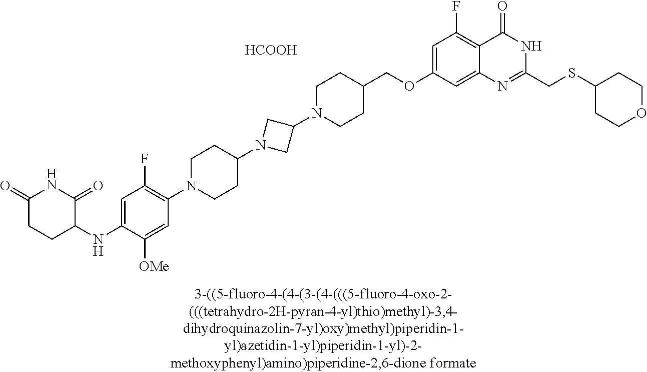 [SMILES: COc1cc(N2CCC(N3CC(N4CCC(COc5cc(F)c6c(=O)[nH]c(CSC7CCOCC7)nc6c5)CC4)C3)CC2)c(F)cc1NC1CCC(=O)NC1=O ; "HCOOH"] | LCMS (ESI, m/z): 796.25 [M + H]⁺; ¹H- NMR (300 MHz, DMSO-d₆) δ 12.15 (s, 1H), 10.83 (s, 1H), 8.18 (s, 1H, HCOOH), 6.94-6.83 (m, 2H), 6.64-6.42 (m, 2H), 5.07 (d, 5.7 Hz, 1H), 4.33-4.15 (m, 1H), 4.07-3.98 (m, 2H), 3.87-3.65 (m, 6H), 3.62 (s, 2H), 3.49- 3.11 (m, 5H), 3.11- 3.03 (m, 3H), 2.89- 2.55 (m, 8H), 2.19- 2.05 (m, 2H), 1.95- 1.75 (m, 9H), 1.50- 1.31 (m, 6H). |

Examples 140-142 were synthesized according to the procedure described for the synthesis 3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1l-yl)phenyl)amino)piperidine-2,6-dione (Example 88; steps C-E) using appropriate building blocks and modified reaction conditions (such as reagents, reagent ratio, temperature, and reaction time) and purification conditions as needed.

| Example | Structure | Analytical Data |
|---|---|---|
| 140 | 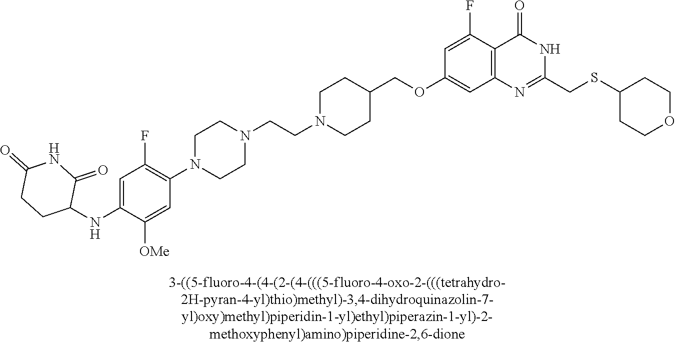 [SMILES: COc1cc(N2CCN(CCN3CCC(COc4cc(F)c5c(=O)[nH]c(CSC6CCOCC6)nc5c4)CC3)CC2)c(F)cc1NC1CCC(=O)NC1=O] | LCMS (ESI, m/z): 770.30 [M + H]⁺. ¹H NMR (300 MHz, Methanol- d4) δ 8.51 (br, 1H), 6.95 (s, 1H), 6.85 (dd, J = 12.5, 2.4 Hz, 1H), 6.66 (d, J = 7.8 Hz, 1H), 6.57 (d, J = 13.8 Hz, 1H), 4.22 (dd, J = 12.3, 4.9 Hz, 1H), 4.09 (d, J = 5.7 Hz, 2H), 3.98- 3.80 (m, 5H), 3.71 (s, 2H), 3.57 (d, J = 11.9 Hz, 2H), 2.50-3.40 (m, 2H), 3.28-3.20 (m, 2H), 3.16-3.08 (m, 5H), 3.00-2.71 |
| | | (m, 10H), 2.48- |
| | | 2.20 (m, 1H), 2.25- |
| | | 2.04 (m, 3H), |
| | | 2.03-1.88 (m, |
| | | 3H), 1.82-1.70 |
| | | (m, 2H), 1.69- |
| | | 1.51 (m, 2H) |
| 141 | 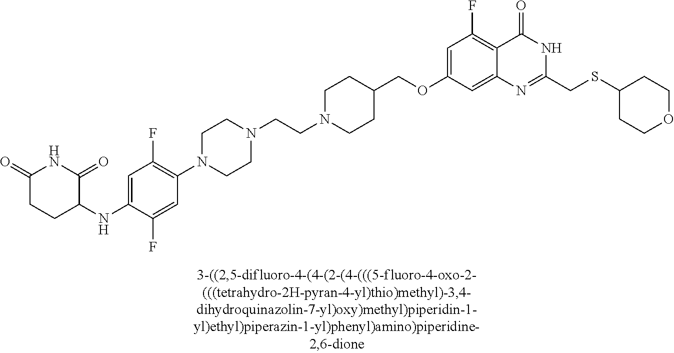 [SMILES: O=C1CCC(Nc2cc(F)c(N3CCN(CCN4CCC(COc5cc(F)c6c(=O)[nH]c(CSC7CCOCC7)nc6c5)CC4)CC3)cc2F)C(=O)N1] | LCMS (ESI, m/z): 758.25 [M + H]⁺; 1H NMR (300 MHz, DMSO-d₆) δ 12.14 (s, 1H), 10.78 (s, 1H), 6.95- 6.67 (m, 4H), 5.47 (d, J = 8.3 Hz, 1H), 4.42-4.27 (m, 1H), 3.98 (d, J = 5.8 Hz, 2H), 3.89-3.76 (m, 2H), 3.62 (s, 2H), 3.39-3.31 (m, 2H), 3.15-2.99 (m, 1H), 3.00- 2.77 (m, 6H), 2.77- 2.65 (m, 1H), 2.64-2.55 (m, 1H), 2.55-2.50 (m, 4H), 2.45- 2.40 (m, 4H), 2.10- |
| | | 1.83 (m, 6H), |
| | | 1.80-1.65 (m, |
| | | 3H), 1.53-1.39 |
| | | (m, 2H), 1.35- |
| | | 1.21 (m, 2H). |
| 142 | 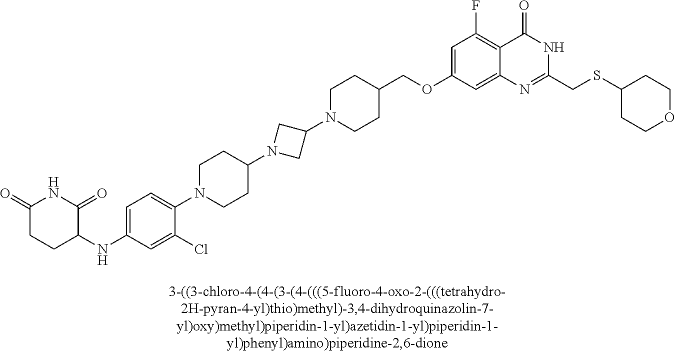 [SMILES: O=C1CCC(Nc2ccc(N3CCC(N4CC(N5CCC(COc6cc(F)c7c(=O)[nH]c(CSC8CCOCC8)nc7c6)CC5)C4)CC3)c(Cl)c2)C(=O)N1] | LCMS (ESI, m/z): 782.30 [M + H]⁺; ¹H NMR (400 MHz, DMSO-d₆) δ 12.10 (s, 1H), 10.76 (s, 1H), 6.97- 6.91 (m, 1H), 6.91-6.84 (m, 2H), 6.72 (s, 1H), 6.62-6.53 (m, 1H), 5.82 (d, J = 7.8 Hz, 1H), 4.34- 4.23 (m, 1H), 4.02- 3.94 (m, 2H), 3.86-3.78 (m, 2H), 3.62 (s, 2H), 3.40-3.36 (m, 2H), 3.11-2.97 (m, 3H), 2.84- 2.67 (m, 6H), 2.62- 2.57 (m, 1H), |
| | | 2.55-2.54 (m, |
| | | 1H), 2.13-2.02 |
| | | (m, 2H), 1.92- |
| | | 1.86 (m, 2H), 1.82- |
| | | 1.66 (m, 7H), |
| | | 1.52-1.40 (m, |
| | | 2H), 1.36-1.26 |
| | | (m, 4H), 1.26- |
| | | 1.21 (m, 3H), 0.87- |
| | | 0.81 (m, 1H). |

### Example 143: Synthesis of 3-((3-fluoro-4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-azaspiro[3.5]nonan-7-yl)phenyl)amino)piperidine-2,6-dione

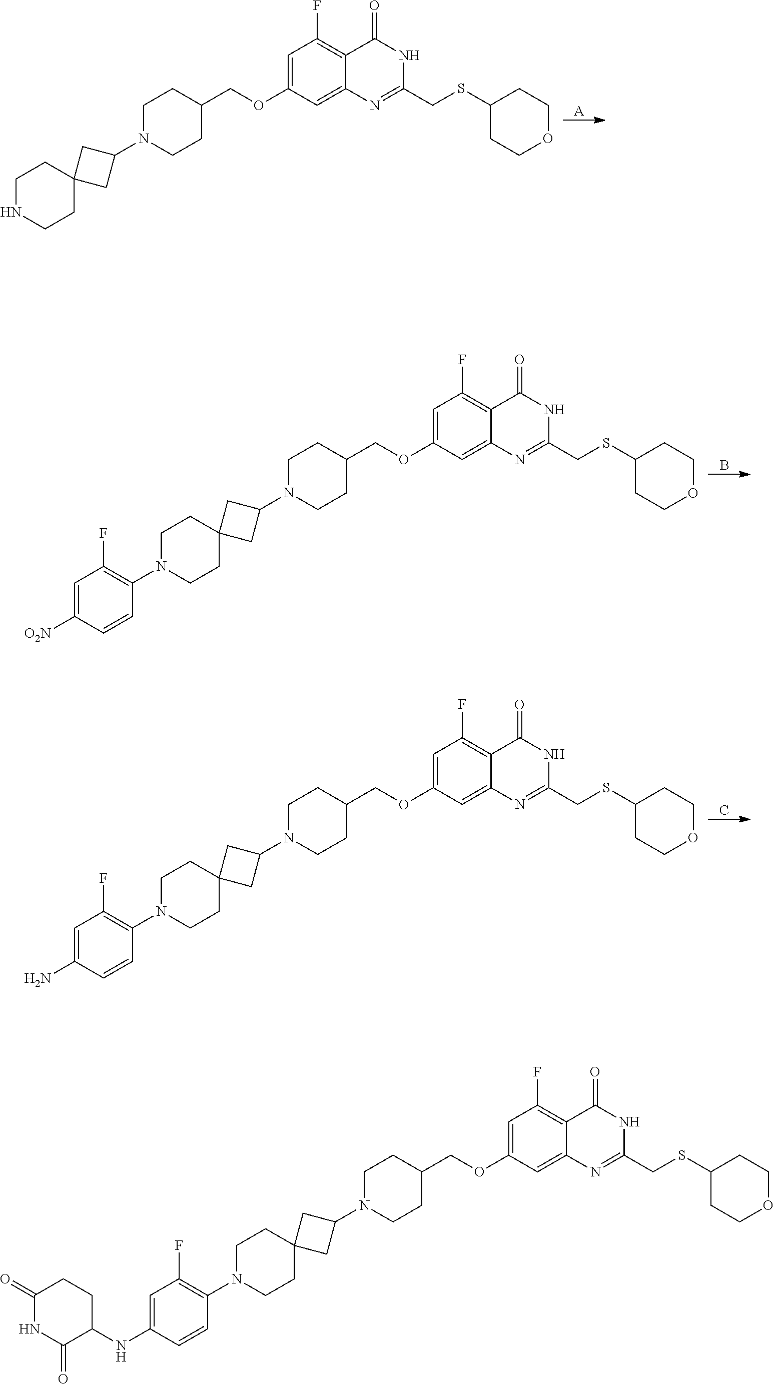

### Step A

A solution of 7-((1-(7-azaspiro[3.5]nonan-2-yl)piperidin-4-yl)methoxy)-5-fluoro-2-(((tetrahydro-2H-pyran-4-yl)thio)methyl)quinazolin-4(3H)-one (3.0 g, 5.7 mmol, 1 equiv), K₂CO₃ (1.22 g, 11.3 mmol, 2 equiv) and 1,2-difluoro-4-nitrobenzene (1.80 g, 11.3 mmol, 2 equiv) in DMF (5 mL) was stirred for 3 hours at 80° C. The resulting mixture was concentrated under reduced pressure. The residue was purified by silica gel column chromatography, eluting with DCM/MeOH (93:7) to afford 5-fluoro-7-((1-(7-(2-fluoro-4-nitrophenyl)-7-azaspiro[3.5]nonan-2-yl)piperidin-4-yl)methoxy)-2-(((tetrahydro-2H-pyran-4-yl)thio)methyl)quinazolin-4(3H)-one (1.37 g, 36%) as a yellow solid. LCMS (ESI, m/z): 670.70 [M+H]⁺.

### Step B

A solution of 5-fluoro-7-((1-(7-(2-fluoro-4-nitrophenyl)-7-azaspiro[3.5]nonan-2-yl)piperidin-4-yl)methoxy)-2-(((tetrahydro-2H-pyran-4-yl)thio)methyl)quinazolin-4(3H)-one (1.35 g, 2.02 mmol, 1 equiv) in EtOH (20 mL) and water (5 mL) was treated with NH₄Cl (431 mg, 8.06 mmol, 4 equiv) and Fe (450 mg, 8.06 mmol, 4 equiv) for 2 hours at 80° C. The resulting mixture was concentrated under reduced pressure. The residue was purified by silica gel column chromatography, eluting with DCM/MeOH (92:8) to afford 7-((1-(7-(4-amino-2-fluorophenyl)-7-azaspiro[3.5]nonan-2-yl)piperidin-4-yl)methoxy)-5-fluoro-2-(((tetrahydro-2H-pyran-4-yl)thio)methyl)quinazolin-4(3H)-one (1.02 g, 79%) as a yellow solid. LCMS (ESI, m/z): 640.20 [M+H]⁺.

### Step C

A solution of 7-((1-(7-(4-amino-2-fluorophenyl)-7-azaspiro[3.5]nonan-2-yl)piperidin-4-yl)methoxy)-5-fluoro-2-(((tetrahydro-2H-pyran-4-yl)thio)methyl)quinazolin-4(3H)-one (500 mg, 0.78 mmol, 1 equiv) in ACN (20 mL) was treated with 3-bromopiperidine-2,6-dione (450 mg, 2.34 mmol, 3 equiv) and NaHCO₃ (328 mg, 3.91 mmol, 5 equiv) for 2 days at 90° C. The resulting mixture was concentrated under reduced pressure. The residue was purified by silica gel column chromatography, eluting with DCM/MeOH (88:12) to afford crude compound. Further purification by Prep-HPLC with the following conditions (Column: YMC-Actus Triart C8, 20*250 mm, 5 μm; Mobile Phase A: water (10 mmol/L NH₄HCO₃), Mobile Phase B: ACN; Flow rate: 60 mL/min; Gradient: isocratic 35%-45%; Wave Length: 254 nm) provided 3-((3-fluoro-4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-azaspiro[3.5]nonan-7-yl)phenyl)amino)piperidine-2,6-dione (95.4 mg, 16%) as a white solid. LCMS (ESI, m/z): 749.55 [M+H]⁺; ¹H NMR (400 MHz, DMSO-d₆) δ 12.14 (s, 1H), 10.77 (s, 1H), 6.93-6.77 (m, 3H), 6.49 (dd, J=14.9, 2.6 Hz, 1H), 6.40 (dd, J=8.6, 2.6 Hz, 1H), 5.77 (d, J=7.6 Hz, 1H), 4.32-4.21 (m, 1H), 3.98 (d, J=5.7 Hz, 2H), 3.82 (dd, J=11.6, 3.7 Hz, 2H), 3.62 (s, 2H), 3.10-3.01 (m, 1H), 2.88-2.54 (m, 9H), 2.16-2.03 (m, 1H), 1.97-1.80 (m, 5H), 1.79-1.62 (m, 7H), 1.60-1.39 (m, 7H), 1.34-1.21 (m, 3H).

### Example 144: Synthesis of 3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)phenyl)amino)piperidine-2,6-dione

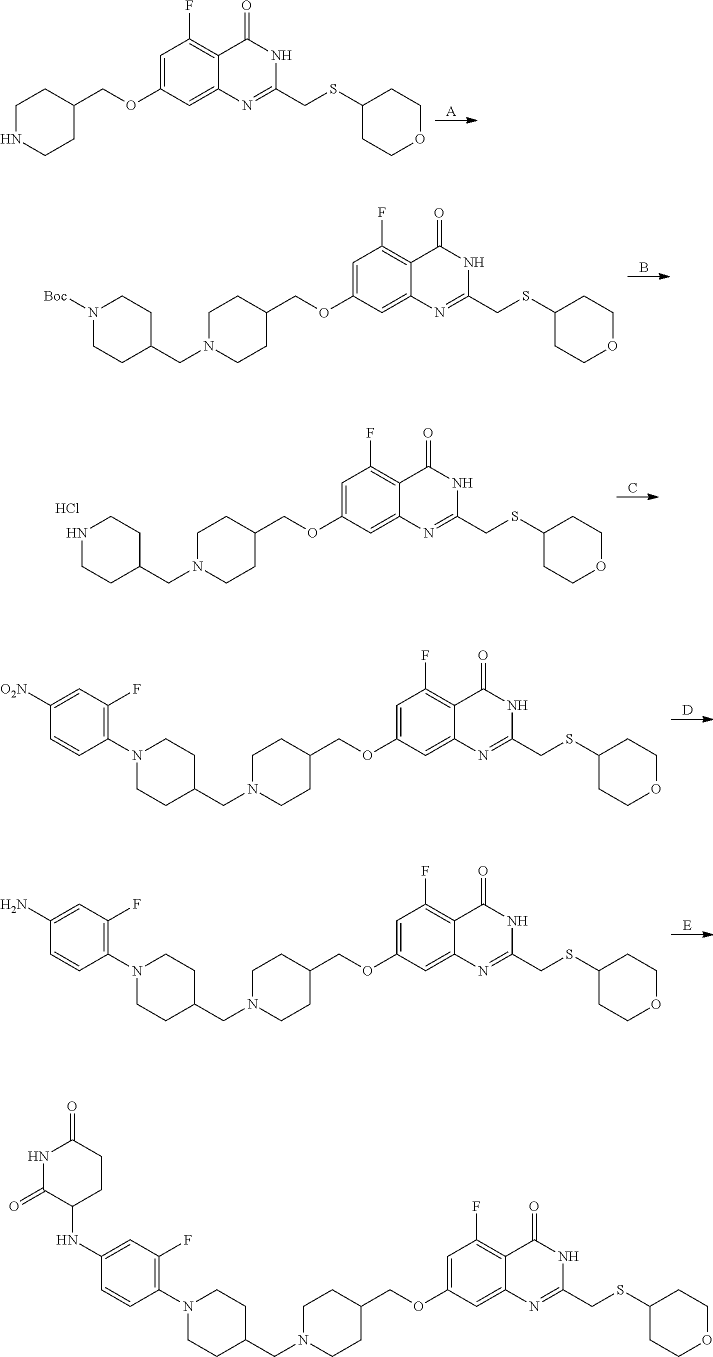

### Step A

A solution of 5-fluoro-7-(piperidin-4-ylmethoxy)-2-(((tetrahydro-2H-pyran-4-yl)thio)methyl)quinazolin-4(3H)-one (500 mg, 1.23 mmol, 1 equiv), tert-butyl 4-formylpiperidine-1-carboxylate (393 mg, 1.84 mmol, 1.5 equiv) and STAB (780 mg, 3.68 mmol, 3 equiv) in DCE (5 mL) was stirred for 1 hour. The solution was concentrated and the residue was purified by silica gel column chromatography, eluting with DCM/MeOH (95:5) to afford tert-butyl 4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidine-1-carboxylate (603 mg, 81%) as a white solid. LCMS (ESI, m/z): 605.50 [M+H]⁺.

### Step B

A solution of tert-butyl 4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidine-1-carboxylate (600 mg, 0.992 mmol, 1 equiv) in HCl in 1,4-dioxane (5 mL, 4M) was stirred for 1 hour. The resulting mixture was concentrated under reduced pressure to give 5-fluoro-7-((1-(piperidin-4-ylmethyl)piperidin-4-yl)methoxy)-2-(((tetrahydro-2H-pyran-4-yl)thio)methyl)quinazolin-4(3H)-one hydrochloride (985 mg) as a white solid. The crude product was used in the next step directly without further purification. LCMS (ESI, m/z): 505.25 [M+H]⁺.

### Step C

A solution of 5-fluoro-7-((1-(piperidin-4-ylmethyl)piperidin-4-yl)methoxy)-2-(((tetrahydro-2H-pyran-4-yl)thio)methyl)quinazolin-4(3H)-one hydrochloride (561 mg, 1.11 mmol, 1 equiv), 1,2-difluoro-4-nitrobenzene (212 mg, 1.33 mmol, 1.2 equiv) and NaHCO₃ (280 mg, 3.34 mmol, 3 equiv) in ACN (4 mL) was stirred for 2 hours at 80° C. The solution was concentrated and the residue was purified by silica gel column chromatography, eluting with DCM/EtOH (95:5) to afford 5-fluoro-7-((1-((1-(2-fluoro-4-nitrophenyl)piperidin-4-yl)methyl)piperidin-4-yl)methoxy)-2-(((tetrahydro-2H-pyran-4-yl)thio)methyl)quinazolin-4(3H)-one (449 mg, 63%) as a yellow solid. LCMS (ESI, m/z): 644.25 [M+H]⁺.

### Step D

A solution of 5-fluoro-7-((1-((1-(2-fluoro-4-nitrophenyl)piperidin-4-yl)methyl)piperidin-4-yl)methoxy)-2-(((tetrahydro-2H-pyran-4-yl)thio)methyl)quinazolin-4(3H)-one (439 mg, 0.682 mmol, 1 equiv), Fe (4.34 mg, 0.080 mmol, 5 equiv) and NH₄Cl (73.0 mg, 1.36 mmol, 2 equiv) in EtOH (5 mL) and water (1 mL) was stirred for 3 hours at 80° C. The resulting mixture was filtered and the filter cake was washed with EtOH (5×10 mL). The filtrate was concentrated under reduced pressure. The residue was purified by silica gel column chromatography, eluting with DCM/MeOH (92:8) to afford 7-((1-((1-(4-amino-2-fluorophenyl)piperidin-4-yl)methyl)piperidin-4-yl)methoxy)-5-fluoro-2-(((tetrahydro-2H-pyran-4-yl)thio)methyl)quinazolin-4(3H)-one (355 mg, 85%) as a yellow solid. LCMS (ESI, m/z): 614.25 [M+H]⁺.

### Step E

A solution of 7-((1-((1-(4-amino-2-fluorophenyl)piperidin-4-yl)methyl)piperidin-4-yl)methoxy)-5-fluoro-2-(((tetrahydro-2H-pyran-4-yl)thio)methyl)quinazolin-4(3H)-one (300 mg, 0.439 mmol, 1 equiv), 3-bromopiperidine-2,6-dione (253 mg, 1.32 mmol, 3 equiv), and NaHCO₃ (185 mg, 2.20 mmol, 5 equiv) in ACN (6 mL) was stirred for 6 hours at 90° C. The resulting mixture was concentrated under reduced pressure. The residue was purified by silica gel column chromatography, eluting with DCM/EtOH (92:8) to afford crude compound (335 mg). The crude product was purified by Prep-HPLC with the following conditions (Column: Kinetex EVO C₁₈, 21.2*250 mm, 5 μm; Mobile Phase A: Water (50 mmol/L NH₄HCO₃), Mobile Phase B: ACN; Flow rate: 60 mL/min; Gradient: 25% B to 39% B in 12 min; Wave Length: 254 nm/220 nm; RT (min): 9.26) to afford 3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)phenyl)amino)piperidine-2,6-dione (23 mg, 6%) as a white solid. LCMS (ESI, m/z): 725.30 [M+H]⁺. ¹H NMR (400 MHz, DMSO-d₆) δ 6.95-6.70 (m, 3H), 6.50 (dd, J=14.9, 2.6 Hz, 1H), 6.41 (dd, J=8.7, 2.6 Hz, 1H), 5.77 (d, J=7.7 Hz, 1H), 4.30-4.20 (m, 1H), 3.98 (d, J=5.8 Hz, 2H), 3.89-3.70 (m, 2H), 3.63 (s, 2H), 3.38-3.34 (m, 1H), 3.20-3.00 (m, 3H), 2.87 (d, J=10.7 Hz, 2H), 2.80-2.66 (m, 1H), 2.63-2.52 (m, 2H), 2.16-1.95 (m, 4H), 1.95-1.68 (m, 10H), 1.65-1.55 (m, 1H), 1.52-1.39 (m, 2H), 1.39-1.18 (m, 5H).

Example 145 was synthesized according to the procedure described for the synthesis afford 3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)phenyl)amino)piperidine-2,6-dione (Example 144) using appropriate building blocks and modified reaction conditions (such as reagents, reagent ratio, temperature, and reaction time) and purification conditions as needed.

| Example | Structure | Analytical Data |
|---|---|---|
| 145 | 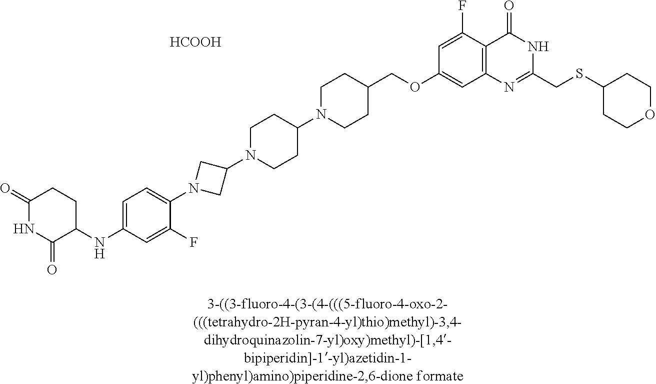 [SMILES: O=C1CCC(Nc2ccc(N3CC(N4CCC(N5CCC(COc6cc(F)c7c(=O)[nH]c(CSC8CCOCC8)nc7c6)CC5)CC4)C3)c(F)c2)C(=O)N1 ; "HCOOH"] | LCMS (ESI, m/z): 766.30 [M + H]+; ¹H NMR (400 MHz, DMSO-d₆) δ 12.16 (s, 1H), 10.76 (s, 1H), 8.19 (s, 1H), 6.92-6.84 (m, 2H), 6.51-6.38 (m, 3H), 5.55 (d, J = 7.5 Hz, 1H), 4.26-4.15 (m, 1H), 3.97 (d, J = 5.8 Hz, 2H), 3.86- 3.79 (m, 4H), 3.62 (s, 2H), 3.51-3.43 (m, 2H), 3.36-3.30 (m, 2H), 3.17- 3.00 (m, 2H), 2.92 (d, J = 10.9 Hz, 2H), 2.82 (d, J = 12.0 Hz, 2H), 2.79- 2.67 (m, 1H), 2.37-2.05 (m, 5H), 1.96-1.70 (m, 10H), 1.51-1.39 (m, 4H), 1.35-1.22 (m, 2H) |

### Example 146: Synthesis of 3-((3-fluoro-4-(4-(3-(4-(((5-fluoro-4-oxo-2-(2-(tetrahydro-2H-pyran-4-yl)ethyl)-3,4-dihydroquinazolin-7-yl)oxy)methyl)piperidin-1-yl)azetidin-1-yl)piperidin-1-yl)phenyl)amino)piperidine-2,6-dione formate

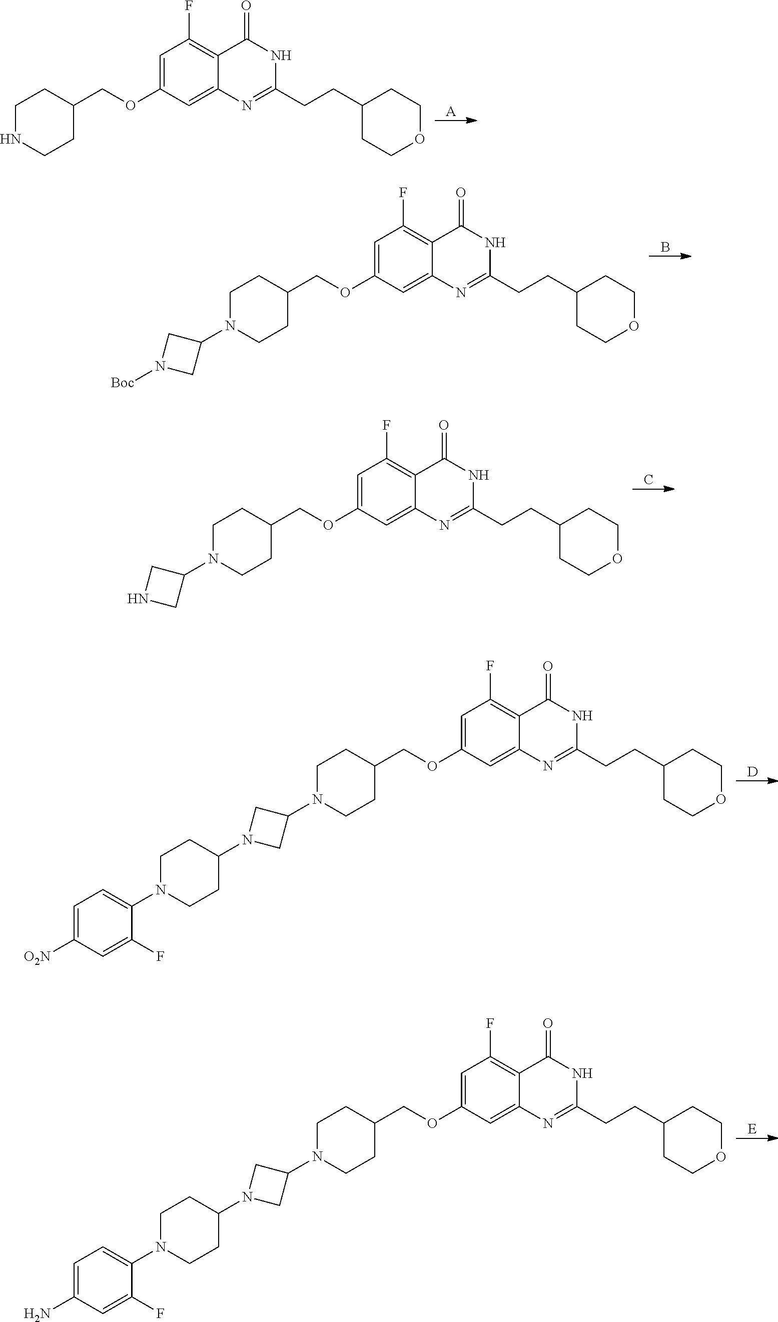

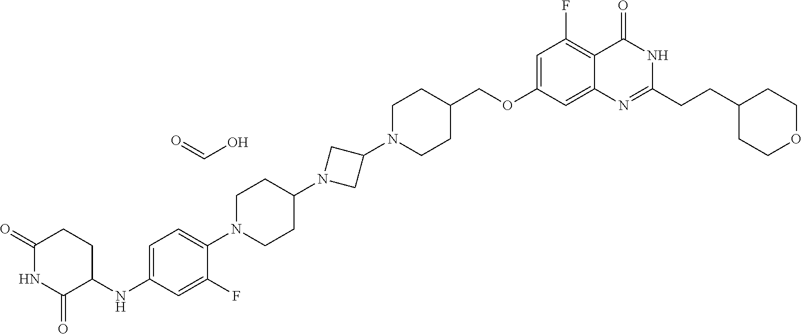 [SMILES: O=C1CCC(Nc2ccc(N3CCC(N4CC(N5CCC(COc6cc(F)c7c(=O)[nH]c(CCC8CCOCC8)nc7c6)CC5)C4)CC3)c(F)c2)C(=O)N1 ; O=CO]

### Step A

A solution of 5-fluoro-2-[2-(oxan-4-yl)ethyl]-7-(piperidin-4-ylmethoxy)-3H-quinazolin-4-one (5.1 g, 13.1 mmol, 1 equiv), tert-butyl 3-oxoazetidine-1-carboxylate (2.69 g, 15.7 mmol, 1.2 equiv) and STAB (5.55 g, 26.2 mmol, 2 equiv) in DCM (25 mL) was stirred for 7 hours. The resulting mixture was concentrated under reduced pressure. The residue was purified by silica gel column chromatography, eluting with DCM/MeOH (94:6) to afford tert-butyl 3-(4-(((5-fluoro-4-oxo-2-(2-(tetrahydro-2H-pyran-4-yl)ethyl)-3,4-dihydroquinazolin-7-yl)oxy)methyl)piperidin-1-yl)azetidine-1-carboxylate (4.2 g, 590%) as a yellow oil. LCMS (ESI, m/z): 545.25 [M+H]⁺.

### Step B

A solution of tert-butyl 3-(4-(((5-fluoro-4-oxo-2-(2-(tetrahydro-2H-pyran-4-yl)ethyl)-3,4-dihydroquinazolin-7-yl)oxy)methyl)piperidin-1-yl)azetidine-1-carboxylate (4.2 g, 7.71 mmol, 1 equiv) and TFA (2 mL) in DCM (10 mL) was stirred for 30 min. The resulting mixture was concentrated under vacuum to afford crude 7-((1-(azetidin-3-yl)piperidin-4-yl)methoxy)-5-fluoro-2-(2-(tetrahydro-2H-pyran-4-yl)ethyl)quinazolin-4(3H)-one (3.2 g, 93%) as a brown oil. LCMS (ESI, m/z):445.30 [M+H]⁺.

### Step C

A solution of 7-((1-(azetidin-3-yl)piperidin-4-yl)methoxy)-5-fluoro-2-(2-(tetrahydro-2H-pyran-4-yl)ethyl)quinazolin-4(3H)-one (3.6 g, 8.1 mmol, 1 equiv), 1-(2-fluoro-4-nitrophenyl)piperidin-4-one (2.89 g, 12.1 mmol, 1.5 equiv) and STAB (3.43 g, 16.2 mmol, 2 equiv) in DCM (20 mL) and MeOH (5 mL) was stirred for 3 hours. The mixture was concentrated under vacuum. The residue was purified by silica gel column chromatography, eluting with DCM/MeOH (10:1) to afford 5-fluoro-7-((1-(1-(1-(2-fluoro-4-nitrophenyl)piperidin-4-yl)azetidin-3-yl)piperidin-4-yl)methoxy)-2-(2-(tetrahydro-2H-pyran-4-yl)ethyl)quinazolin-4(3H)-one (5.2 g, 96%) as a brown solid. LCMS (ESI, m/z): 667.30 [M+H]⁺.

### Step D

A solution of 5-fluoro-7-((1-(1-(1-(2-fluoro-4-nitrophenyl)piperidin-4-yl)azetidin-3-yl)piperidin-4-yl)methoxy)-2-(2-(tetrahydro-2H-pyran-4-yl)ethyl)quinazolin-4(3H)-one (3.0 g, 4.5 mmol, 1 equiv) and Fe (1.26 g, 0.225 mmol, 5 equiv), and NH₄Cl (0.48 g, 9.0 mmol, 2 equiv) in EtOH (10 mL) and water (2 mL) was stirred for 3 hours at 80° C. The residue was purified by silica gel column chromatography, eluting with DCM/MeOH (8:1) to afford 7-((1-(1-(1-(4-amino-2-fluorophenyl)piperidin-4-yl)azetidin-3-yl)piperidin-4-yl)methoxy)-5-fluoro-2-(2-(tetrahydro-2H-pyran-4-yl)ethyl)quinazolin-4(3H)-one (2.5 g, 87%) as a brown solid. LCMS (ESI, m/z): 637.40 [M+H]⁺.

### Step E

A solution of 7-((1-(1-(1-(4-amino-2-fluorophenyl)piperidin-4-yl)azetidin-3-yl)piperidin-4-yl)methoxy)-5-fluoro-2-(2-(tetrahydro-2H-pyran-4-yl)ethyl)quinazolin-4(3H)-one (120 mg, 0.188 mmol, 1 equiv), 3-bromopiperidine-2,6-dione (43.4 mg, 0.226 mmol, 1.2 equiv) and NaHCO₃ (39.6 mg, 0.470 mmol, 2.5 equiv) in NMP (2 mL) was stirred overnight at 85° C. The residue was purified by reverse-phase flash chromatography with the following conditions: column, C18 silica gel; mobile phase, MeCN in water (0.1% FA), 10% to 25% gradient in 10 min; detector, UV 254 nm to afford 3-((3-fluoro-4-(4-(3-(4-(((5-fluoro-4-oxo-2-(2-(tetrahydro-2H-pyran-4-yl)ethyl)-3,4-dihydroquinazolin-7-yl)oxy)methyl)piperidin-1-yl)azetidin-1-yl)piperidin-1-yl)phenyl)amino)piperidine-2,6-dione formate (72.8 mg, 52%) as a dark grey solid. LCMS (ESI, m/z): 748.40 [M+H]⁺. ¹H NMR (400 MHz, DMSO-d₆) δ 11.94 (s, 1H), 10.77 (s, 1H), 8.21 (s, 1H), 6.90-6.78 (m, 3H), 6.50 (dd, J=14.9, 2.6 Hz, 1H), 6.41 (dd, J=8.7, 2.5 Hz, 1H), 5.78 (d, J=7.6 Hz, 1H), 4.30-4.20 (m, 1H), 3.97 (d, J=5.9 Hz, 2H), 3.86-3.81 (m, 3H), 3.45 (s, 2H), 3.30-3.24 (m, 2H), 3.07 (d, J=11.4 Hz, 2H), 2.86-2.82 (m, 2H), 2.78-2.69 (m, 3H), 2.60-2.59 (m, 1H), 2.58-2.56 (m, 2H), 2.56-2.54 (m, 3H), 2.20-2.12 (m, 1H), 2.11-2.06 (m, 1H), 2.04-1.98 (m, 1H), 1.80-1.58 (m, 9H), 1.54-1.45 (m, 1H), 1.35-1.15 (m, 7H).

### Example 147: Synthesis of 3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)(methyl)amino)phenyl)amino)piperidine-2,6-dione

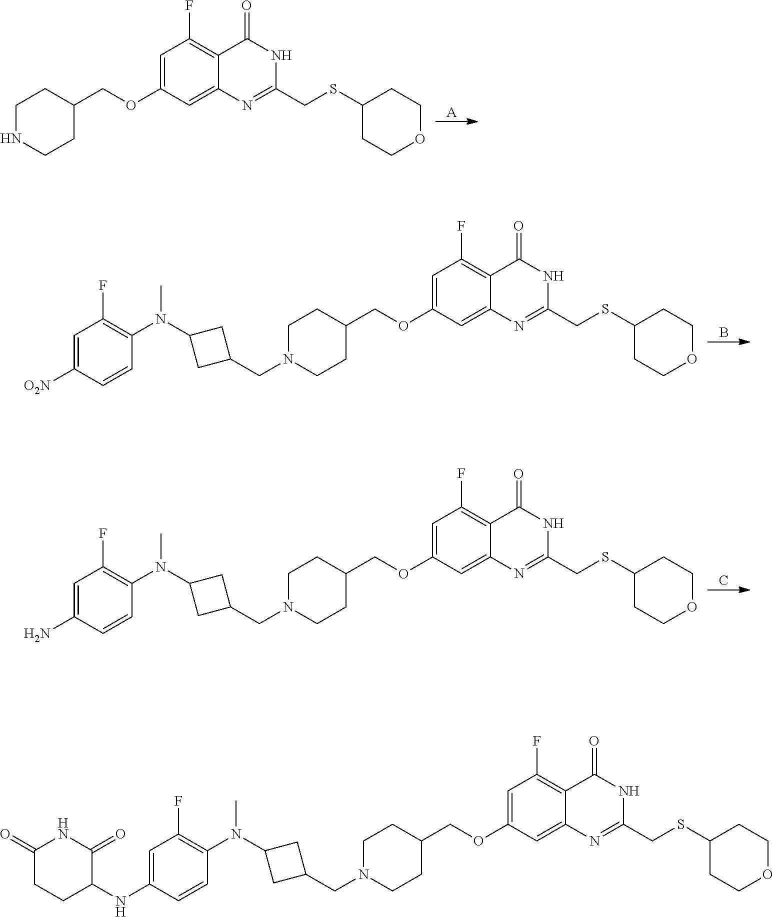

### Step A

A solution of (3-((2-fluoro-4-nitrophenyl)(methyl)amino)cyclobutyl)methyl 4-methylbenzenesulfonate (663 mg, 1.62 mmol, 1 equiv), 5-fluoro-2-[(oxan-4-ylsulfanyl)methyl]-7-(piperidin-4-ylmethoxy)-3H-quinazolin-4-one (794 mg, 1.95 mmol, 1.2 equiv), KI (27 mg, 0.16 mmol, 0.1 equiv) and DIEA (629 mg, 4.87 mmol, 3 equiv) in ACN (15 mL) was stirred for 15 hours at 80° C. The resulting mixture was concentrated under reduced pressure. The residue was purified by silica gel column chromatography, eluting with DCM/MeOH (22:3), afforded 5-fluoro-7-((1-((3-((2-fluoro-4-nitrophenyl)(methyl)amino)cyclobutyl)methyl)piperidin-4-yl)methoxy)-2-(((tetrahydro-2H-pyran-4-yl)thio)methyl)quinazolin-4(3H)-one (684 mg, 65%) as a yellow oil. LCMS (ESI, m/z): 644.20[M+H]⁺.

### Step B

A solution of 5-fluoro-7-((1-((3-((2-fluoro-4-nitrophenyl)(methyl)amino)cyclobutyl)methyl)piperidin-4-yl)methoxy)-2-(((tetrahydro-2H-pyran-4-yl)thio)methyl)quinazolin-4(3H)-one (664 mg, 1.03 mmol, 1 equiv) in EtOH (16 mL) was treated with NH₄Cl (110 mg, 2.06 mmol, 2 equiv) in water (4 mL) at room temperature followed by the addition of Fe (288 mg, 5.16 mmol, 5 equiv) portionwise at 80° C. The resulting mixture was stirred for 2 hours at 80° C. and then concentrated under reduced pressure. The residue was purified by silica gel column chromatography, eluting with DCM/MeOH (43:7) to afford 7-((1-((3-((4-amino-2-fluorophenyl)(methyl)amino)cyclobutyl)methyl)piperidin-4-yl)methoxy)-5-fluoro-2-(((tetrahydro-2H-pyran-4-yl)thio)methyl)quinazolin-4(3H)-one (484 mg, 76%) as a yellow solid. LCMS (ESI, m/z): 614.40 [M+H]⁺.

### Step C

A solution of 7-((1-((3-((4-amino-2-fluorophenyl)(methyl)amino)cyclobutyl)methyl)piperidin-4-yl)methoxy)-5-fluoro-2-(((tetrahydro-2H-pyran-4-yl)thio)methyl)quinazolin-4(3H)-one (460 mg, 0.75 mmol, 1 equiv), 3-bromopiperidine-2,6-dione (432 mg, 2.25 mmol, 3 equiv) and NaHCO₃ (315 mg, 3.75 mmol, 5 equiv) in ACN (20 mL) was stirred for three days at 90° C. The resulting mixture was concentrated under reduced pressure. The residue was purified by silica gel column chromatography, eluting with DCM/EtOH (41:9), and further purified by Prep-HPLC with the following conditions: (Column: Xselect CSH OBD Column 30*150 mm, 5 umn; Mobile Phase A: water (0.1% FA), Mobile Phase B: ACN; Flow rate: 60 mL/min; Gradient: 5% B to 5% B in 2 min, 5% B to 15% B in 10 min; Wave Length: 254 nm/220 nm; RT (min): 9.2) to afford 3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)(methyl)amino)phenyl)amino)piperidine-2,6-dione formate (95 mg, 16%) as a white solid. LCMS (ESI, m/z): 725.30 [M+H]⁺, ¹H-NMR (400 MHz, DMSO-d₆) δ 12.15 (s, 1H), 10.76 (s, 1H), 8.19 (s, 1H) HCOOH, 6.91-6.82 (m, 2H), 6.81-6.69 (m, 1H), 6.48 (d, J=14.0 Hz, 1H), 6.40 (d, J=8.0 Hz, 1H), 5.82 (d, J=8.0 Hz, 1H), 4.32-4.16 (m, 1H), 4.01-3.91 (m, 2H), 3.88-3.75 (m, 2H), 3.62 (s, 2H), 3.45-3.39 (m, 1H), 3.37-3.32 (m, 2H), 3.10-3.00 (m, 1H), 2.88 (d, J=8.0 Hz, 2H), 2.78-2.68 (m, 1H), 2.62-2.56 (m, 1H), 2.48-2.46 (m, 3H), 2.40-2.30 (m, 2H), 2.27-1.99 (m, 6H), 1.94-1.69 (m, 7H), 1.53-1.40 (m, 4H), 1.38-1.24 (m, 2H).

### Example 148: Synthesis of 3-((3-fluoro-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-[1,3′-biazetidin]-1′-yl)phenyl)amino)piperidine-2,6-dione formate

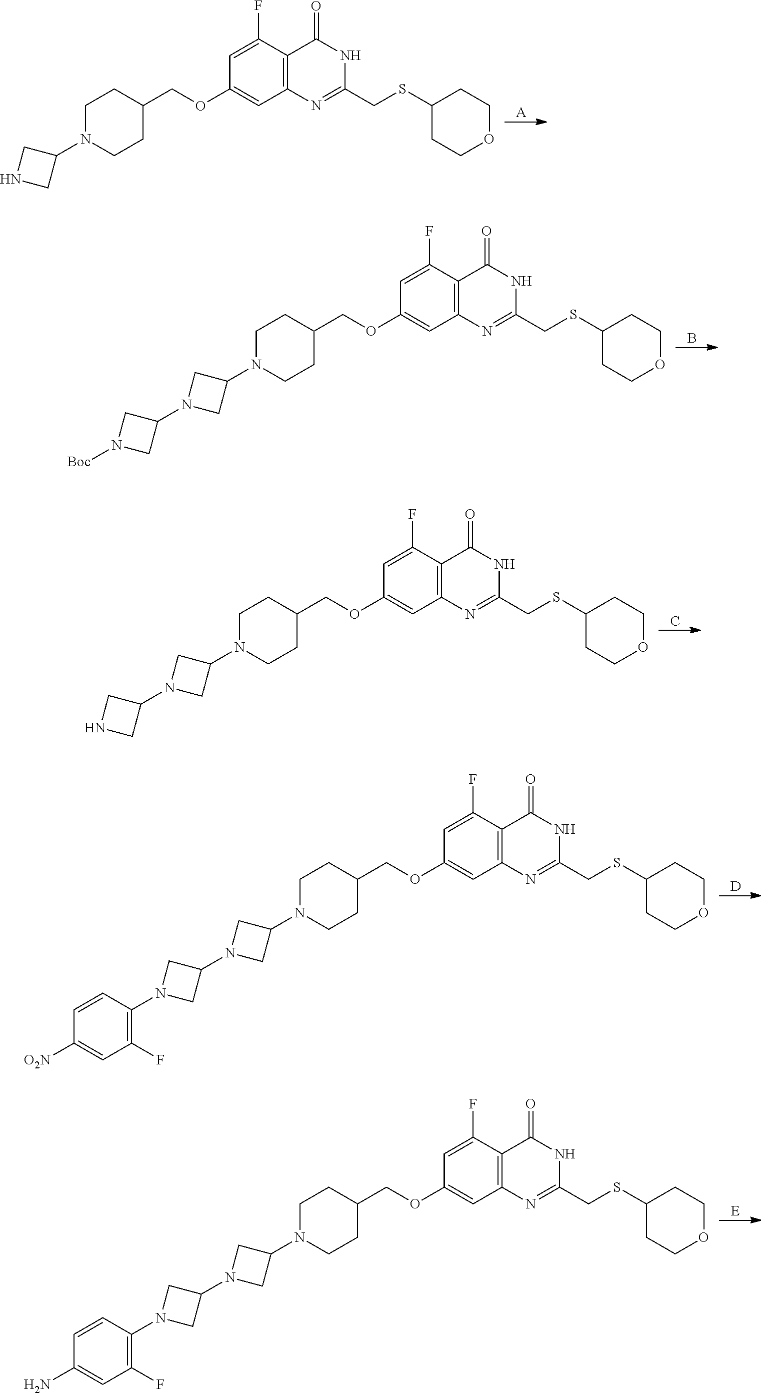

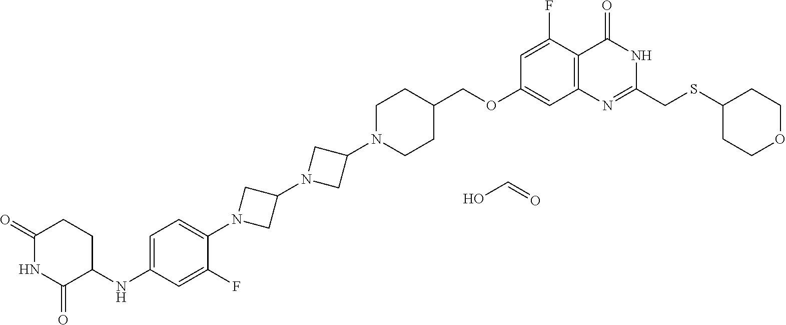 [SMILES: O=C1CCC(Nc2ccc(N3CC(N4CC(N5CCC(COc6cc(F)c7c(=O)[nH]c(CSC8CCOCC8)nc7c6)CC5)C4)C3)c(F)c2)C(=O)N1 ; O=CO]

### Step A

A solution of 7-((1-(azetidin-3-yl)piperidin-4-yl)methoxy)-5-fluoro-2-(((tetrahydro-2H-pyran-4-yl)thio)methyl)quinazolin-4(3H)-one (1.4 g, 3.03 mmol, 1 equiv), tert-butyl 3-oxoazetidine-1-carboxylate (1.04 g, 6.05 mmol, 2 equiv) and STAB (3.21 g, 15.1 mmol, 5 equiv) in DCE (20 mL) was stirred for 2 hours. The resulting mixture was concentrated under reduced pressure. The residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm to give tert-butyl 3-(4-(((5-fluoro-4-oxo-2-(((tetrahydro-2H-pyran-4-yl)thio)methyl)-3,4-dihydroquinazolin-7-yl)oxy)methyl)piperidin-1-yl)-[1,3′-biazetidine]-1′-carboxylate (1.6 g, 86%) as a white solid. LCMS (ESI, m/z): 618.10 [M+H]⁺.

### Step B

A solution of tert-butyl 3-(4-(((5-fluoro-4-oxo-2-(((tetrahydro-2H-pyran-4-yl)thio)methyl)-3,4-dihydroquinazolin-7-yl)oxy)methyl)piperidin-1-yl)-[1,3′-biazetidine]-1′-carboxylate (1.6 g, 2.59 mmol, 1 equiv) in TFA (2 mL) and DCM (10 mL) was stirred for 2 hours. The resulting mixture was concentrated under reduced pressure to give crude 7-((1-([1,3′-biazetidin]-3-yl)piperidin-4-yl)methoxy)-5-fluoro-2-(((tetrahydro-2H-pyran-4-yl)thio)methyl)quinazolin-4(3H)-one (2.8 g) as a yellow oil. The crude product was used in next step without further purification. LCMS (ESI, m/z): 518.10 [M+H]⁺.

### Step C

A mixture of 7-((1-([1,3′-biazetidin]-3-yl)piperidin-4-yl)methoxy)-5-fluoro-2-(((tetrahydro-2H-pyran-4-yl)thio)methyl)quinazolin-4(3H)-one (2.3 g, 4.44 mmol, 1 equiv), 1,2-difluoro-4-nitrobenzene (1.06 g, 6.66 mmol, 1.5 equiv) and NaHCO₃ (1.49 g, 17.8 mmol, 4 equiv) in MeCN (10 mL) was stirred for 3 hours at 80° C. The resulting mixture was concentrated under reduced pressure. The residue was purified by silica gel column chromatography, eluting with DCM/MeOH (15:1) to afford 5-fluoro-7-((1-(1′-(2-fluoro-4-nitrophenyl)-[1,3′-biazetidin]-3-yl)piperidin-4-yl)methoxy)-2-(((tetrahydro-2H-pyran-4-yl)thio)methyl)quinazolin-4(3H)-one (650 mg, 22%) as a yellow solid. LCMS (ESI, m/z): 657.10 [M+H]⁺.

### Step D

A solution of 5-fluoro-7-((1-(1′-(2-fluoro-4-nitrophenyl)-[1,3′-biazetidin]-3-yl)piperidin-4-yl)methoxy)-2-(((tetrahydro-2H-pyran-4-yl)thio)methyl)quinazolin-4(3H)-one (540 mg, 0.822 mmol, 1 equiv) in DMF (5 mL) was treated with B₂(OH)₄ (221 mg, 2.47 mmol, 3 equiv) for 1 min followed by the addition of 4,4′-bipyridine (12.8 mg, 0.082 mmol, 0.1 equiv). The resulting mixture was concentrated under reduced pressure. The residue was purified by reverse-phase flash chromatography with the following conditions: column, C18 silica gel; mobile phase, MeCN in water (10 mmol/L NH₄HCO₃), 50% to 50% gradient in 5 min; detector, UV 254 nm. This resulted in crude 7-((1-(1′-(4-amino-2-fluorophenyl)-[1,3′-biazetidin]-3-yl)piperidin-4-yl)methoxy)-5-fluoro-2-(((tetrahydro-2H-pyran-4-yl)thio)methyl)quinazolin-4(3H)-one (560 mg) as a white solid. LCMS (ESI, m/z): 627.10 [M+H]⁺.

### Step E

A solution of 7-((1-(1′-(4-amino-2-fluorophenyl)-[1,3′-biazetidin]-3-yl)piperidin-4-yl)methoxy)-5-fluoro-2-(((tetrahydro-2H-pyran-4-yl)thio)methyl)quinazolin-4(3H)-one (200 mg, 0.319 mmol, 1 equiv), 3-bromopiperidine-2,6-dione (92 mg, 0.48 mmol, 1.5 equiv) and NaHCO₃ (130 mg, 1.6 mmol, 5 equiv) in DMF (5 ml) was stirred for 15 hours at 85° C. The resulting mixture was concentrated under reduced pressure. The residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. The resulting impure product was purified by Prep-HPLC with the following conditions (Column: XBridge Prep Phenyl Column, 19*250 mm, 5 μm; Mobile Phase A: Water (0.1% FA), Mobile Phase B: ACN; Flow rate: 25 mL/min; Gradient: 3% B to 30% B in 9 min; Wave Length: 254 nm/220 nm; RT (min): 9) to afford 3-((3-fluoro-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-[1,3′-biazetidin]-1′-yl)phenyl)amino)piperidine-2,6-dione formate (20 mg, 9%) as a yellow solid. LCMS (ESI, m/z): 738.30 [M+H]⁺; ¹H NMR (300 MHz, DMSO-d₆) δ 12.15 (s, 1H), 10.74 (s, 1H), 8.14 (s, 1H), 6.97-6.83 (m, 2H), 6.52-6.37 (m, 3H), 5.54 (d, J=7.5 Hz, 1H), 4.31-4.02 (m, 2H), 3.99 (d, J=5.8 Hz, 2H), 3.88-3.72 (m, 4H), 3.61 (s, 2H), 3.59-3.49 (m, 3H), 3.48-3.41 (m, 3H), 3.39-3.37 (m, 1H), 3.14-2.88 (m, 5H), 2.84-2.69 (m, 3H), 2.63-2.58 (m, 1H), 2.16-2.04 (m, 1H), 1.92-1.74 (m, 6H), 1.55-1.22 (m, 4H).

### Example 149: Synthesis of N-(2,6-dioxopiperidin-3-yl)-2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)benzamide

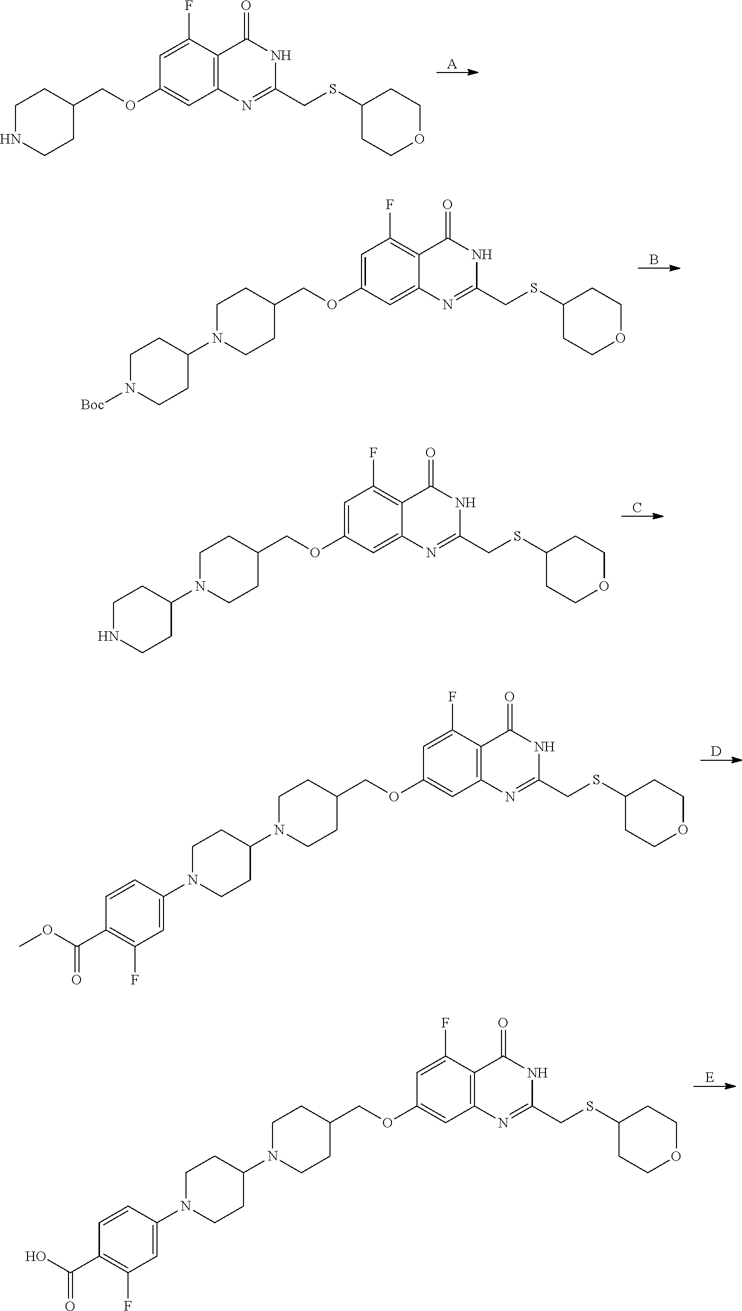

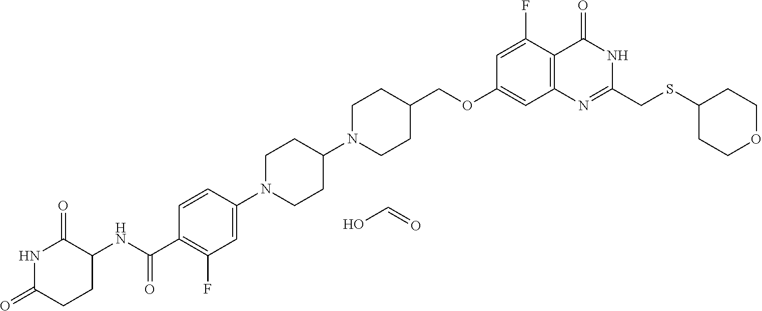 [SMILES: O=C1CCC(NC(=O)c2ccc(N3CCC(N4CCC(COc5cc(F)c6c(=O)[nH]c(CSC7CCOCC7)nc6c5)CC4)CC3)cc2F)C(=O)N1 ; O=CO]

### Step A

A solution of 5-fluoro-7-(piperidin-4-ylmethoxy)-2-(((tetrahydro-2H-pyran-4-yl)thio)methyl)quinazolin-4(3H)-one (5 g, 12 mmol, 1 equiv) in DCE (100 mL) was treated with tert-butyl tert-butyl 4-oxopiperidine-1-carboxylate (12.2 g, 61.4 mmol, 5 equiv) for 2 hours followed by the addition of STAB (15.6 g, 73.6 mmol, 6 equiv) portionwise and then stirred for 2 days. The solution was concentrated and the residue was purified by silica gel column chromatography, eluting with DCM/MeOH (94:6) to afford tert-butyl 4-(((5-fluoro-4-oxo-2-(((tetrahydro-2H-pyran-4-yl)thio)methyl)-3,4-dihydroquinazolin-7-yl)oxy)methyl)-[1,4′-bipiperidine]-1′-carboxylate (6.1 g, 84%) as a yellow solid. LCMS (ESI, m/z): 591.35 [M+H]⁺.

### Step B

A solution of tert-butyl 4-(((5-fluoro-4-oxo-2-(((tetrahydro-2H-pyran-4-yl)thio)methyl)-3,4-dihydroquinazolin-7-yl)oxy)methyl)-[1,4′-bipiperidine]-1′-carboxylate (3.5 g, 5.93 mmol, 1 equiv) in HCl in 1,4-dioxane (20 mL, 4M) was stirred for 30 min. The resulting mixture was concentrated under reduced pressure to give 7-([1,4′-bipiperidin]-4-ylmethoxy)-5-fluoro-2-(((tetrahydro-2H-pyran-4-yl)thio)methyl)quinazolin-4(3H)-one (5.9 g) as a white solid. The crude material was carried on without purification. LCMS (ESI, m/z): 491.35 [M+H]⁺.

### Step C

A solution of 7-([1,4′-bipiperidin]-4-ylmethoxy)-5-fluoro-2-(((tetrahydro-2H-pyran-4-yl)thio)methyl)quinazolin-4(3H)-one (1.5 g, 3.06 mmol, 1 equiv), methyl 2,4-difluorobenzoate (0.53 g, 3.06 mmol, 1 equiv) and K₂CO₃ (1.27 g, 9.17 mmol, 3 equiv) in ACN (50 mL) was stirred overnight at 70° C. The resulting mixture was concentrated under reduced pressure. The residue was purified by silica gel column chromatography, eluting with DCM/MeOH (9:1) to afford a mixture of two regio-isomers containing methyl 2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)benzoate (231 mg, 12%) as a light yellow solid. LCMS (ESI, m/z): 643.35 [M+H]⁺.

### Step D

A solution of the mixture of regio-isomers containing methyl 2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl) (221 mg, 0.344 mmol, 1 equiv) and NaOH (55.0 mg, 1.38 mmol, 4 equiv) in water (20 mL) was stirred for 15 hours. The mixture was neutralized to pH 7 with HCl solution. The resulting mixture was concentrated under reduced pressure. The residue was purified by silica gel column chromatography, eluting with DCM/MeOH (9:1) to afford the mixture of two regio-isomers containing 2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)benzoic acid (205 mg, 95%) as a brown solid. LCMS (ESI, m/z):629.30 [M+H]⁺.

### Step E

A solution of the mixture of regio-isomers containing 2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)benzoic acid (195 mg, 0.310 mmol, 1 equiv) and 3-aminopiperidine-2,6-dione (40 mg, 0.31 mmol, 1 equiv) in DMF (10 mL) was treated with DIEA (120 mg, 0.93 mmol, 3 equiv) for 2 min. HATU (142 mg, 0.372 mmol, 1.2 equiv) was then added portionwise. The solution was stirred for 2 hours. The residue was purified by reverse-phase flash chromatography with the following conditions: column, C18 silica gel; mobile phase, MeCN in water (0.1% FA), 0% to 70% gradient in 30 min; detector, UV 254 nm. The crude product (136 mg) was purified by Prep-HPLC with the following conditions (Column: Xselect CSH OBD Column 30*150 mm, 5 um; Mobile Phase A: water (0.1% FA), Mobile Phase B: ACN; Flow rate: 20 mL/min; Gradient: 5% B to 5% B in 2 min, 11% B to 21% B in 10 min; Wave Length: 254 nm/220 nm) to afford N-(2,6-dioxopiperidin-3-yl)-2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)benzamide formate (7 mg, 3%). LCMS (ESI, m/z): 739.30 [M+H]⁺; ¹H NMR (400 MHz, DMSO-d₆) δ 12.17 (s, 1H), 10.95 (s, 1H), 9.72 (d, J=6.9 Hz, 1H), 8.19 (s, 1H), 7.84 (dd, J=8.7, 7.0 Hz, 1H), 7.10 (dd, J=11.2, 2.6 Hz, 1H), 7.03-6.94 (m, 1H), 6.93-6.84 (m, 2H), 4.80-4.69 (m, 1H), 3.99 (d, J=5.7 Hz, 2H), 3.87-3.78 (m, 2H), 3.39-3.28 (m, 3H), 3.25-3.18 (m, 2H), 3.10-3.02 (m, 1H), 2.94-2.83 (m, 2H), 2.84-2.66 (m, 3H), 2.61-2.53 (m, 1H), 2.47-2.38 (m, 1H), 2.30-2.18 (m, 3H), 2.08-2.00 (m, 1H), 1.93-1.74 (m, 8H), 1.70-1.59 (m, 1H), 1.53-1.38 (m, 2H), 1.38-1.26 (m, 2H).

### Example 150: Synthesis of 3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)amino)phenyl)amino)piperidine-2,6-dione formate

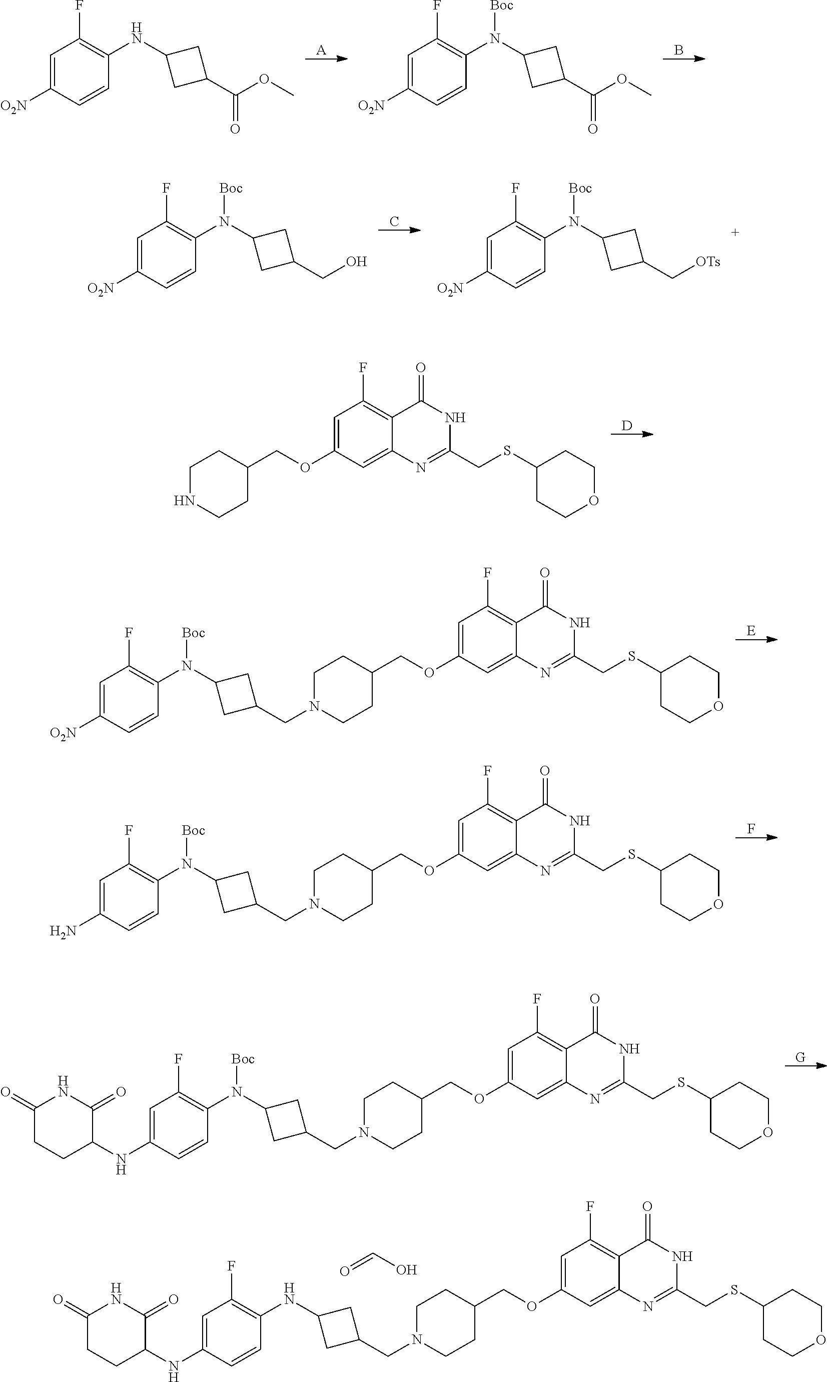

### Step A

A solution of methyl 3-((2-fluoro-4-nitrophenyl)amino)cyclobutane-1-carboxylate (2.0 g, 7.5 mmol, 1 equiv), di-tert-butyl dicarbonate (3.25 g, 14.9 mmol, 2 equiv) and TEA (1.51 g, 14.9 mmol, 2 equiv) in DCM (10 mL) was stirred overnight. The solution was concentrated and the residue was purified by silica gel column chromatography, eluting with PE/EtOAc (1:1) to afford methyl 3-((tert-butoxycarbonyl)(2-fluoro-4-nitrophenyl)amino)cyclobutane-1-carboxylate (2.2 g, 80%) as a white solid. LCMS (ESI, m/z): 368.20 [M+H]⁺.

### Step B

A solution of methyl 3-((tert-butoxycarbonyl)(2-fluoro-4-nitrophenyl)amino)cyclobutane-1-carboxylate (2.2 g, 6.0 mmol, 1 equiv), NaBH₄ (0.45 g, 11.9 mmol, 2 equiv), and CaCl₂) (1.33 g, 11.9 mmol, 2 equiv) in EtOH (10 mL) was stirred overnight. The reaction was quenched with ice water at 0° C. The mixture was concentrated and the residue purified by silica gel column chromatography, eluting with PE/EtOAc (7:3) to afford tert-butyl (2-fluoro-4-nitrophenyl)(3-(hydroxymethyl)cyclobutyl)carbamate (1.6 g, 79%) as a yellow oil. LCMS (ESI, m/z): 340.05 [M+H]⁺.

### Step C

A solution of tert-butyl (2-fluoro-4-nitrophenyl)(3-(hydroxymethyl)cyclobutyl)carbamate (1.1 g, 3.2 mmol, 1 equiv), TsCl (0.92 g, 4.9 mmol, 1.5 equiv), TEA (981 mg, 9.70 mmol, 3 equiv) and DMAP (72 mg, 0.59 mmol, 0.4 equiv) in DCM (10 mL) was stirred for 1 hour. The mixture was concentrated and residue purified by silica gel column chromatography, eluting with PE/EtOAc (88:12) to afford (3-((tert-butoxycarbonyl)(2-fluoro-4-nitrophenyl)amino)cyclobutyl)methyl 4-methylbenzenesulfonate (1.5 g, 85%) as a yellow oil. LCMS (ESI, m/z): 495.35 [M+H]⁺.

### Step D

A solution of (3-((tert-butoxycarbonyl)(2-fluoro-4-nitrophenyl)amino)cyclobutyl)methyl 4-methylbenzenesulfonate (1.46 g, 2.95 mmol, 1.2 equiv), 5-fluoro-7-(piperidin-4-ylmethoxy)-2-(((tetrahydro-2H-pyran-4-yl)thio)methyl)quinazolin-4(3H)-one (1.0 g, 2.5 mmol, 1.0 equiv), KI (41 mg, 0.25 mmol, 0.10 equiv) and K₂CO₃ (678 mg, 4.91 mmol, 2.0 equiv) in ACN (8 mL) was stirred overnight at 80° C. The mixture was concentrated and residue was purified by silica gel column chromatography, eluting with CH₂Cl₂/MeOH (95:5) to afford tert-butyl (2-fluoro-4-nitrophenyl)(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)carbamate (949 mg, 33%) as a yellow solid. LCMS (ESI, m/z): 730.10 [M+H]⁺.

### Step E

A solution of tert-butyl (2-fluoro-4-nitrophenyl)(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)carbamate (500 mg, 0.685 mmol, 1 equiv), Fe (191 mg, 3.43 mmol, 5 equiv), and NH₄Cl (73 mg, 1.37 mmol, 2 equiv) in EtOH (5 mL) and water (1 mL) was stirred for 1 hour at 80° C. The solution was filtered and the cake was washed with EtOH (5×10 mL). The filtrate was concentrated under reduced pressure and residue purified by silica gel column chromatography, eluting with CH₂Cl₂/MeOH (96:4) to afford tert-butyl (4-amino-2-fluorophenyl)(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)carbamate (300 mg, 57%) as a yellow solid. LCMS (ESI, m/z): 700.40 [M+H]⁺.

### Step F

A solution of tert-butyl (4-amino-2-fluorophenyl)(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)carbamate (0.300 g, 0.429 mmol, 1 equiv), 3-bromopiperidine-2,6-dione (247 mg, 1.29 mmol, 3 equiv), and NaHCO₃ (180 mg, 2.15 mmol, 5 equiv) in ACN (3 mL) was stirred overnight at 90° C. The mixture was concentrated and residue purified by silica gel column chromatography, eluting with CH₂Cl₂/EtOH (89:11), to afford tert-butyl (4-((2,6-dioxopiperidin-3-yl)amino)-2-fluorophenyl)(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)carbamate (214 mg, 62%) as a green solid. LCMS (ESI, m/z): 811.55 [M+H]⁺.

### Step G

A solution of tert-butyl (4-((2,6-dioxopiperidin-3-yl)amino)-2-fluorophenyl)(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)carbamate (105 mg, 0.129 mmol, 1 equiv) in HCl in 1,4-dioxane (3 mL, 4 M) was stirred for 1 hour. The mixture was concentrated and the crude product (103 mg) was purified by Prep-HPLC with the following conditions (Column: Xselect CSH OBD Column 30*150 mm, 5 μm; Mobile Phase A: water (0.1% FA), Mobile Phase B: ACN; Flow rate: 60 mL/min; Gradient: 5% B to 5% B in 2 min, 5% B to 15% B in 10 min; Wavelength: 254 nm/220 nm; RT (min): 8.5) to afford 3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)amino)phenyl)amino)piperidine-2,6-dione (10 mg, 11%) as a yellow solid. LCMS (ESI, m/z): 711.20 [M+H]⁺. 1H NMR (400 MHz, DMSO-d6) δ 12.16 (s, 1H), 10.75 (s, 1H), 8.18 (s, 1H), 6.92-6.84 (m, 2H), 6.56-6.43 (m, 2H), 6.42-6.32 (m, 1H), 5.37 (d, J=7.3 Hz, 1H), 4.60 (br, 1H), 4.20-4.10 (m, 1H), 3.98 (d, J=5.8 Hz, 2H), 3.86-3.76 (m, 2H), 3.66-3.58 (m, 3H), 3.48-3.43 (m, 1H), 3.10-3.01 (m, 1H), 2.87 (d, J=10.9 Hz, 2H), 2.78-2.64 (m, 2H), 2.63-2.57 (m, 1H), 2.49-2.33 (m, 4H), 2.20-2.05 (m, 2H), 2.02-1.61 (m, 8H), 1.57-1.38 (m, 4H), 1.35-1.22 (m, 2H).

### Example 151: Synthesis of N-(2,6-dioxopiperidin-3-yl)-2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzamide

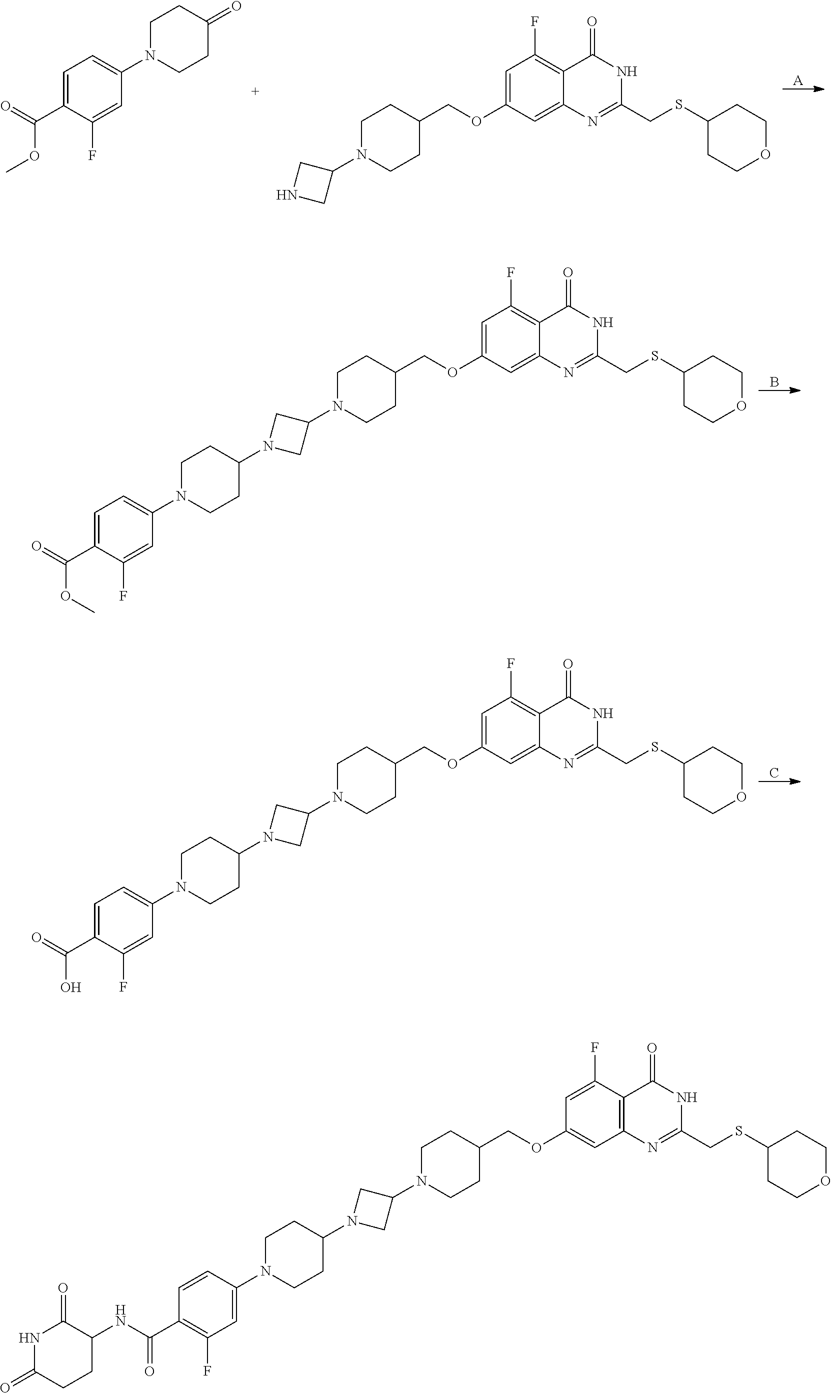

### Step A

A solution of methyl 2-fluoro-4-(4-oxopiperidin-1-yl)benzoate (812 mg, 3.23 mmol, 1 equiv), 7-((1-(azetidin-3-yl)piperidin-4-yl)methoxy)-5-fluoro-2-(((tetrahydro-2H-pyran-4-yl)thio)methyl)quinazolin-4(3H)-one (1.49 g, 3.23 mmol, 1 equiv) and STAB (822 mg, 3.88 mmol, 1.2 equiv) in DCE (8 mL) was stirred for 2 hours at 80° C. The mixture was concentrated, and the residue purified by silica gel column chromatography, eluting with DCM/MeOH (90:10) to afford methyl 2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzoate (600 mg, 27%) as a pink solid. LCMS (ESI, m/z): 698.31 [M+H]⁺.

### Step B

A solution of methyl 2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzoate (0.640 g, 0.917 mmol, 1 equiv) and NaOH (294 mg, 7.34 mmol, 8 equiv) in water (5 mL) was stirred for 3 hours. The mixture was acidified to pH 6 with conc. HCl. The precipitated solids were collected by filtration and washed with water (1×20 mL) to give 2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzoic acid (446 mg, 71%) as an orange solid. LCMS (ESI, m/z): 684.30 [M+H]⁺.

### Step C

A solution of 2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzoic acid (0.150 g, 0.219 mmol, 1 equiv), 3-aminopiperidine-2,6-dione (28.1 mg, 0.219 mmol, 1 equiv), DIEA (85.1 mg, 0.657 mmol, 3 equiv) and HATU (0.100 g, 0.263 mmol, 1.2 equiv) in DMF (3 mL) was stirred for 1 hour. The residue was purified by reverse-phase flash chromatography with the following conditions: column, C18 silica gel; mobile phase, MeCN in water (10 mmol/L NH₄HCO₃), 10% to 40% gradient in 25 min; detector, UV 254 nm to afford N-(2,6-dioxopiperidin-3-yl)-2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzamide (51 mg, 29%) as a white solid. LCMS (ESI, m/z): 794.30 [M+H]⁺. 1H NMR (400 MHz, DMSO-d6) δ 12.15 (s, 1H), 10.83 (s, 1H), 8.00 (t, J=7.3 Hz, 1H), 7.62 (t, J=9.0 Hz, 1H), 6.92-6.85 (m, 2H), 6.84-6.71 (m, 2H), 4.80-4.61 (m, 1H), 4.01-3.95 (m, 2H), 3.87-3.80 (m, 2H), 3.76-3.68 (m, 2H), 3.62 (s, 2H), 3.45-3.40 (m, 2H), 3.38-3.35 (m, 2H), 3.10-3.02 (m, 1H), 3.00-2.98 (m, 2H), 2.90-2.70 (m, 6H), 2.36-2.25 (m, 1H), 2.15-2.08 (m, 1H), 2.06-2.00 (m, 1H), 1.93-1.86 (m, 2H), 1.83-1.67 (m, 7H), 1.50-1.40 (m, 2H), 1.34-1.19 (m, 5H).

### Example 152: Synthesis of 3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carbonyl)piperidin-1-yl)phenyl)amino)piperidine-2,6-dione formate

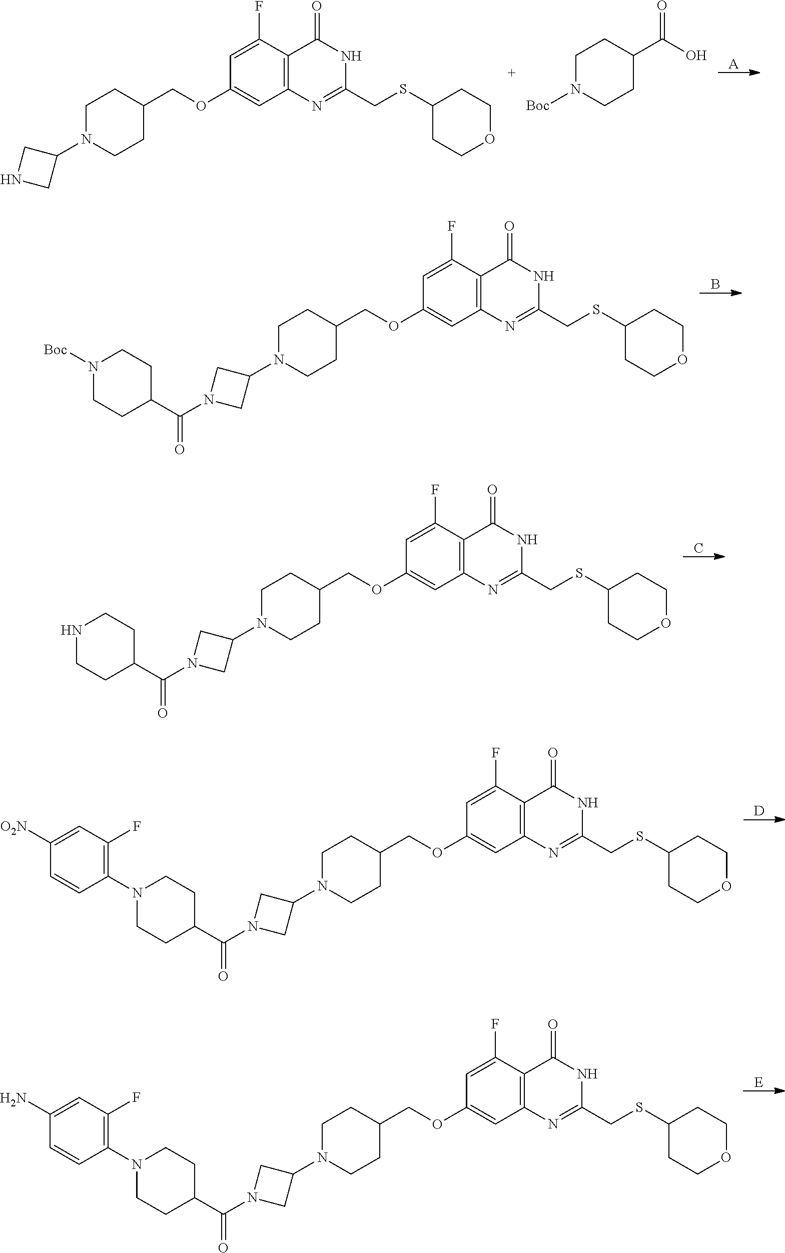

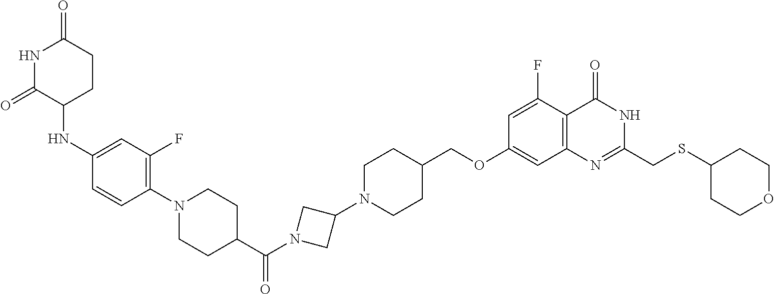 [SMILES: O=C1CCC(Nc2ccc(N3CCC(C(=O)N4CC(N5CCC(COc6cc(F)c7c(=O)[nH]c(CSC8CCOCC8)nc7c6)CC5)C4)CC3)c(F)c2)C(=O)N1]

### Step A

A solution of 7-((1-(azetidin-3-yl)piperidin-4-yl)methoxy)-5-fluoro-2-(((tetrahydro-2H-pyran-4-yl)thio)methyl)quinazolin-4(3H)-one (1.0 g, 2.2 mmol, 1.0 equiv), 1-(tert-butoxycarbonyl)piperidine-4-carboxylic acid (0.50 g, 2.2 mmol, 1.0 equiv), HATU (0.99 g, 2.59 mmol, 1.2 equiv) and DIEA (0.84 g, 6.49 mmol, 3.0 equiv) in DMF (15 mL) was stirred for 4 hours. The reaction was quenched with water and extracted with EtOAc (3×100 mL). The combined organic layers were washed with brine (3×50 mL), dried over anhydrous Na₂SO₄, and filtered. The filtrate was concentrated under reduced pressure. The residue was purified by reverse-phase flash chromatography with the following conditions: column, C18 silica gel; mobile phase, MeCN in water (10 mmol/L NH₄HCO₃), 10% to 60% gradient in 30 min; detector, UV 254 nm to give tert-butyl 4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carbonyl)piperidine-1-carboxylate (600 mg, 41%) as a yellow solid. LCMS (ESI, m/z): 674.40 [M+H]⁺.

### Step B

A solution of tert-butyl 4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carbonyl)piperidine-1-carboxylate (600 mg, 2.27 mmol, 1 equiv) in HCl in 1,4-dioxane (10 mL, 4 M) was stirred for 30 min. The mixture was concentrated under reduced pressure to afford 5-fluoro-7-((1-(1-(piperidine-4-carbonyl)azetidin-3-yl)piperidin-4-yl)methoxy)-2-(((tetrahydro-2H-pyran-4-yl)thio)methyl)quinazolin-4(3H)-one hydrochloride (680 mg) as a white solid. The product was used in the next step directly without further purification. LCMS (ESI, m/z): 574.30 [M+H]⁺.

### Step C

A solution of 5-fluoro-7-((1-(1-(piperidine-4-carbonyl)azetidin-3-yl)piperidin-4-yl)methoxy)-2-(((tetrahydro-2H-pyran-4-yl)thio)methyl)quinazolin-4(3H)-one hydrochloride (660 mg, 1.15 mmol, 1.0 equiv), 1,2-difluoro-4-nitrobenzene (183 mg, 1.15 mmol, 1.0 equiv) and TEA (349 mg, 3.45 mmol, 3.0 equiv) in DMF (6 mL) was stirred for 2 hours at 80° C. The reaction was concentrated, and the residue was purified by reverse-phase flash chromatography with the following conditions: column, C18 silica gel; mobile phase, MeCN in water (10 mmol/L NH₄HCO₃), 10% to 60% gradient in 30 min; detector, UV 254 nm. This afforded 5-fluoro-7-((1-(1-(1-(2-fluoro-4-nitrophenyl)piperidine-4-carbonyl)azetidin-3-yl)piperidin-4-yl)methoxy)-2-(((tetrahydro-2H-pyran-4-yl)thio)methyl)quinazolin-4(3H)-one (0.420 g, 51%) as a yellow solid. LCMS (ESI, m/z): 713.25 [M+H]⁺.

### Step D

A solution of 5-fluoro-7-((1-(1-(1-(2-fluoro-4-nitrophenyl)piperidine-4-carbonyl)azetidin-3-yl)piperidin-4-yl)methoxy)-2-(((tetrahydro-2H-pyran-4-yl)thio)methyl)quinazolin-4(3H)-one (0.400 g, 0.561 mmol, 1 equiv) in EtOH (8 mL) was treated with NH₄Cl (0.06 g, 1.1 mmol, 2 equiv) in water (2 mL) at room temperature followed by the addition of Fe (157 mg, 2.81 mmol, 5 equiv) in portions at 80° C. The mixture was stirred for 2 hours at 80° C. The mixture was concentrated and the residue was purified by silica gel column chromatography, eluting with DCM/MeOH (22:3) to afford 7-((1-(1-(1-(4-amino-2-fluorophenyl)piperidine-4-carbonyl)azetidin-3-yl)piperidin-4-yl)methoxy)-5-fluoro-2-(((tetrahydro-2H-pyran-4-yl)thio)methyl)quinazolin-4(3H)-one (356 mg, 93%) as a yellow solid. LCMS (ESI, m/z): 683.25 [M+H]⁺.

### Step E

A solution of 7-((1-(1-(1-(4-amino-2-fluorophenyl)piperidine-4-carbonyl)azetidin-3-yl)piperidin-4-yl)methoxy)-5-fluoro-2-(((tetrahydro-2H-pyran-4-yl)thio)methyl)quinazolin-4(3H)-one (336 mg, 0.492 mmol, 1 equiv), 3-bromopiperidine-2,6-dione (284 mg, 1.48 mmol, 3 equiv) and NaHCO₃ (207 mg, 2.46 mmol, 5 equiv) in ACN (10 mL) was stirred for overnight at 90° C. The mixture was concentrated and the residue was purified by reverse phase flash chromatography with the following conditions: column, C18 silica gel; mobile phase, MeCN in water (0.1% FA), 10% to 30% gradient in 20 min; detector, UV 254 nm to give 3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carbonyl)piperidin-1-yl)phenyl)amino)piperidine-2,6-dione formate (93 mg, 23%) as a white solid. LCMS (ESI, m/z): 794.25 [M+H]⁺. 1H NMR (400 MHz, DMSO-d₆) δ 12.16 (s, 1H), 10.76 (s, 1H), 8.15 (s, 1H) HCOOH, 6.93-6.78 (m, 3H), 6.51 (dd, J=14.9, 2.6 Hz, 1H), 6.42 (dd, J=8.7, 2.6 Hz, 1H), 5.79 (d, J=7.6 Hz, 1H), 4.31-4.16 (m, 2H), 4.03-3.96 (m, 3H), 3.90-3.78 (m, 3H), 3.69-3.61 (m, 3H), 3.38-3.28 (m, 2H), 3.17-3.02 (m, 4H), 2.88-2.76 (m, 2H), 2.76-2.65 (m, 1H), 2.64-2.52 (m, 3H), 2.35-2.24 (m, 1H), 2.15-2.04 (m, 1H), 1.93-1.73 (m, 8H), 1.71-1.58 (m, 4H), 1.53-1.39 (m, 2H), 1.38-1.26 (m, 2H).

Example 153 was synthesized according to the procedure described for the synthesis 3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carbonyl)piperidin-1-yl)phenyl)amino)piperidine-2,6-dione formate (Example 152) using appropriate building blocks and modified reaction conditions (such as reagents, reagent ratio, temperature, and reaction time) and purification conditions as needed.

| Example | Structure | Analytical Data |
|---|---|---|
| Example 153 | 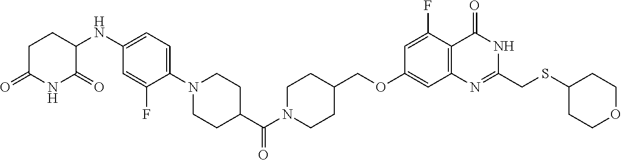 [SMILES: O=C1CCC(Nc2ccc(N3CCC(C(=O)N4CCC(COc5cc(F)c6c(=O)[nH]c(CSC7CCOCC7)nc6c5)CC4)CC3)c(F)c2)C(=O)N1] 3-((3-fluoro-4-(4-(4-(((5-fluoro-4-oxo-2-(((tetrahydro- 2H-pyran-4-yl)thio)methyl)-3,4-dihydroquinazolin-7- yl)oxy)methyl)piperidine-1-carbonyl)piperidin-1- yl)phenyl)amino)piperidine-2,6-dione | LCMS (ESI, m/z): 739.25 [M + H]⁺. 1H NMR (300 MHz, DMSO-d6) δ 12.14 (br, 1H), 10.79 (s, 1H), 7.20-7.00 (m, 2H), 6.87 (d, J = 2.4 Hz, 2H), 6.60-6.40 (m, 2H), 5.39-4.60 (m, 2H), 4.59-4.25 (m, 2H), 4.15-3.95 (m, 3H), 3.94-3.75 (m, 2H), 3.65 (s, 2H), 3.49-3.25 (m, 4H), 3.19-3.02 (m, 3H), 2.94-2.69 (m, 2H), 2.68-2.52 (m, 2H), 2.20-2.01 (m, 2H), 1.98-1.61 (m, 9H), 1.58-1.49 (m, 2H), 1.48-0.81 (m, 2H). |

### Example 154: Synthesis of 3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperazin-1-yl)phenyl)amino)piperidine-2,6-dione

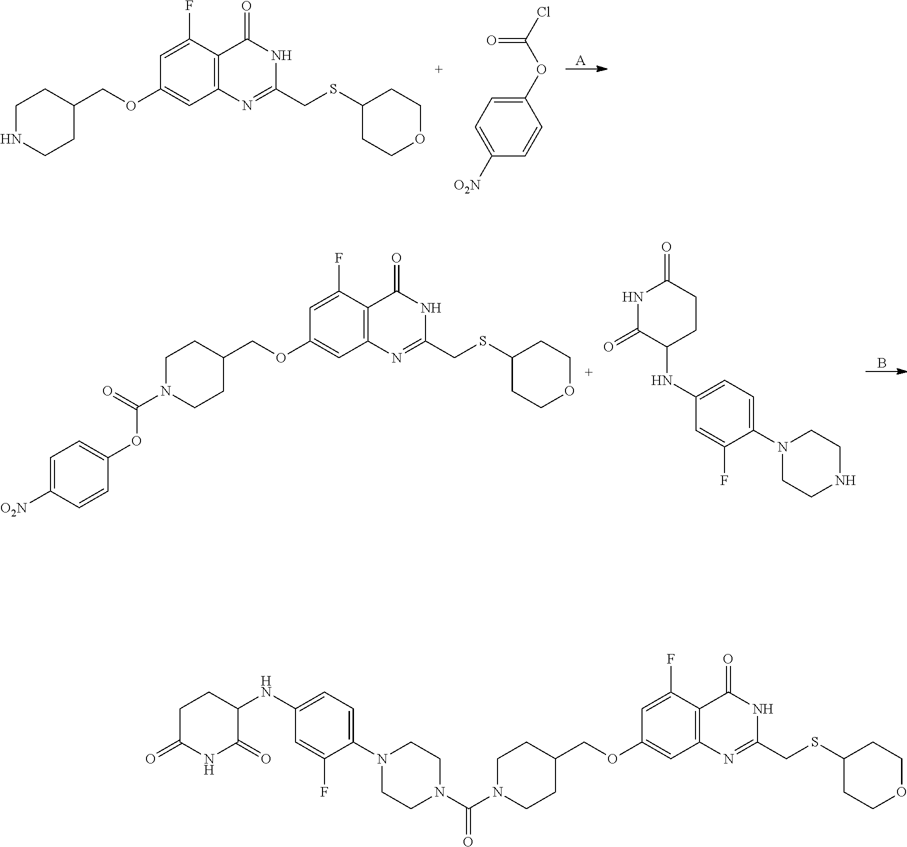

### Step A

A solution of 5-fluoro-2-[(oxan-4-ylsulfanyl)methyl]-7-(piperidin-4-ylmethoxy)-3H-quinazolin-4-one (1.70 g, 4.17 mmol, 2.1 equiv) and 4-nitrophenyl chloroformate (0.800 g, 3.97 mmol, 2 equiv) in pyridine (10 mL) was stirred for 1 hour at 70° C. The mixture was concentrated and the residue was purified by reverse phase flash chromatography with the following conditions: (column, C18 silica gel; mobile phase, MeCN in water (0.1% FA), 10% to 50% gradient in 10 min; detector, UV 254 nm) to afford 4-nitrophenyl 4-(((5-fluoro-4-oxo-2-(((tetrahydro-2H-pyran-4-yl)thio)methyl)-3,4-dihydroquinazolin-7-yl)oxy)methyl)piperidine-1-carboxylate. (0.280 g, 25%) as a white solid. LCMS (ESI, m/z): 572.15 [M+H]⁺.

### Step B

A solution of 4-nitrophenyl 4-(((5-fluoro-4-oxo-2-(((tetrahydro-2H-pyran-4-yl)thio)methyl)-3,4-dihydroquinazolin-7-yl)oxy)methyl)piperidine-1-carboxylate (230 mg, 0.402 mmol, 1 equiv), 3-((3-fluoro-4-(piperazin-1-yl)phenyl)amino)piperidine-2,6-dione (615 mg, 2.01 mmol, 5 equiv) and DIEA (259 mg, 2.01 mmol, 2.0 equiv) in DMSO (3 mL) was stirred for 24 hours at 100° C. The mixture was purified directly by reverse phase flash with the following conditions (ACN:water=40:60) and further purified by Prep-HPLC with the following conditions (Column: Xselect CSH OBD Column 30*150 mm, 5 μm; Mobile Phase A: water (0.1% FA), Mobile Phase B: ACN; Flow rate: 60 mL/min; Gradient: 5% B to 5% B in 2 min, 25% B to 35% B in 10 min; Wavelength: 254 nm/220 nm; RT (min): 9.2) to afford 3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperazin-1-yl)phenyl)amino)piperidine-2,6-dione (19 mg, 6%) as a white solid. LCMS (ESI, m/z): 740.45 [M+H]⁺. 1H NMR (300 MHz, DMSO-d6) δ 12.18 (br, 1H), 10.79 (s, 1H), 6.89 (d, J=9.2 Hz, 3H), 6.53 (d, J=15.0 Hz, 1H) 6.44 (d, J=9.0 Hz, 1H), 4.28 (d, J=11.3 Hz, 1H), 4.01 (d, J=6.2 Hz, 2H), 3.82 (d, J=11.8 Hz, 2H), 3.72-3.66 (m, 1H), 3.66-3.59 (m, 4H), 3.40-3.24 (m, 7H), 2.91-2.66 (m, 7H), 2.60-2.51 (m, 1H), 2.15-1.70 (m, 7H), 1.51-1.35 (m, 2H), 1.30-1.15 (m, 2H).

### Example 155: Synthesis of 1-(6-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione

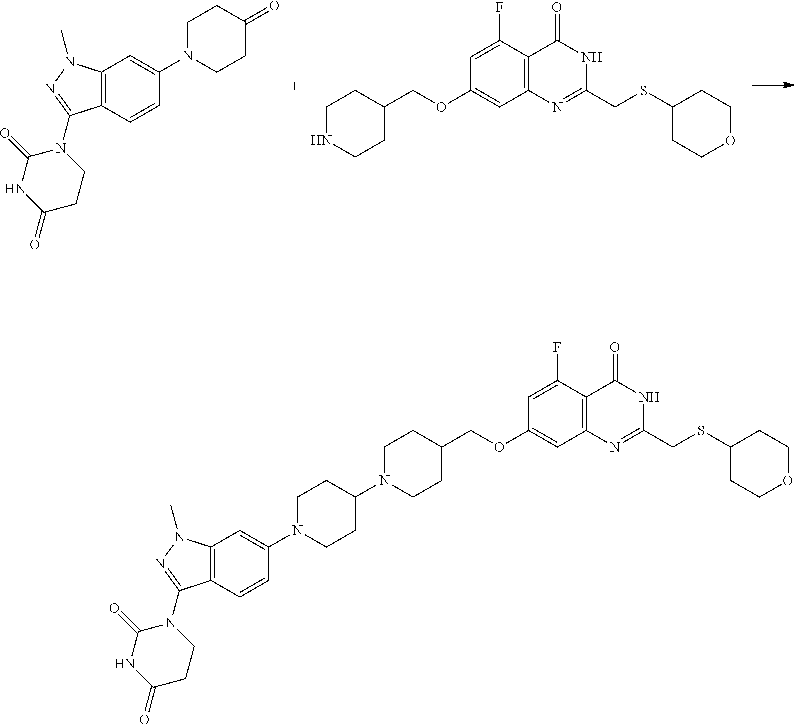 [SMILES: Cn1nc(N2CCC(=O)NC2=O)c2ccc(N3CCC(=O)CC3)cc21 ; O=c1[nH]c(CSC2CCOCC2)nc2cc(OCC3CCNCC3)cc(F)c12 ; Cn1nc(N2CCC(=O)NC2=O)c2ccc(N3CCC(N4CCC(COc5cc(F)c6c(=O)[nH]c(CSC7CCOCC7)nc6c5)CC4)CC3)cc21]

A solution of 1-(1-methyl-6-(4-oxopiperidin-1-yl)-1H-indazol-3-yl)dihydropyrimidine-2,4(1H,3H)-dione (135 mg, 0.395 mmol, 1 equiv), 5-fluoro-7-(piperidin-4-ylmethoxy)-2-(((tetrahydro-2H-pyran-4-yl)thio)methyl)quinazolin-4(3H)-one (161 mg, 0.395 mmol, 1 equiv) and STAB (168 mg, 0.790 mmol, 2 equiv) in DCE (20 mL) was stirred for 2 hours at 40° C. The mixture was concentrated and the residue purified by reverse phase flash chromatography with the following conditions: column, C18 silica gel; mobile phase, MeCN in water (10 mmol/L NH₄HCO₃), 10% to 50% gradient in 10 min; detector, UV 254 nm to give 1-(6-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione (18 mg, 6%) as a white solid. LCMS (ESI, m/z): 733.35 [M+H]⁺. 1H NMR (400 MHz, DMSO-d₆) δ 12.05 (br, 1H), 10.50 (s, 1H), 7.44 (d, J=9.0 Hz, 1H), 6.97-6.84 (m, 3H), 6.82 (s, 1H), 3.98 (d, J=5.6 Hz, 2H), 3.94-3.79 (m, 9H), 3.62 (s, 2H), 3.12-3.00 (m, 1H), 2.94-2.86 (m, 2H), 2.85-2.70 (m, 4H), 2.32-2.28 (m, 1H), 2.24-2.05 (m, 2H), 1.98-1.68 (m, 8H) 1.65-1.58 (m, 2H), 1.57-1.45 (m, 3H), 1.34-1.22 (m, 2H).

Examples 156-169 were synthesized according to the procedure described for the synthesis 1-(6-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione Example 155 using appropriate building blocks and modified reaction conditions (such as reagents, reagent ratio, temperature, and reaction time) and purification conditions as needed.

| Example | Structure | Analytical Data |
|---|---|---|
| Example 156 | 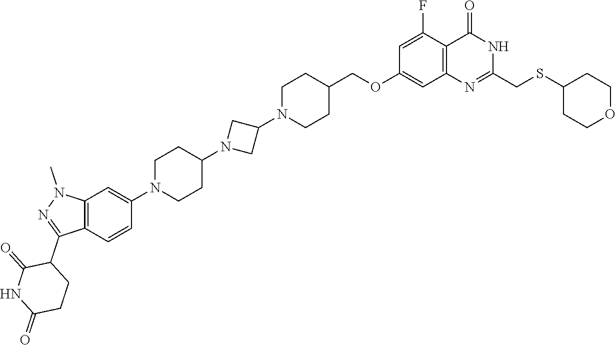 [SMILES: Cn1nc(C2CCC(=O)NC2=O)c2ccc(N3CCC(N4CC(N5CCC(COc6cc(F)c7c(=O)[nH]c(CSC8CCOCC8)nc7c6)CC5)C4)CC3)cc21] 3-(6-(4-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)azetidin-1-yl)piperidin- 1-yl)-1-methyl-1H-indazol-3-yl)piperidine-2,6-dione | LCMS (ESI, m/z): 787.40 [M + H]⁺. 1H NMR (400 MHz, DMSO-d6) δ 12.18 (s, 1H), 10.85 (s, 1H), 7.49 (d, J = 8.2 Hz, 1H), 6.94- 6.83 (m, 4H), 4.30- 4.22 (m, 1H), 3.99 (d, J = 8.0 Hz, 2H), 3.89 (s, 3H), 3.82 (d, J = 8.0 Hz, 2H), 3.79-3.70 (m, 2H), 3.62 (s, 3H), 3.30-3.28 (m, 2H), 3.13-3.01 (m, 2H), 2.99-2.91 (m, 1H), 2.85-2.75 (m, 4H), 2.70- 2.55 (m, 3H), 2.33- 2.26 (m, 1H), 2.22- 2.11 (m, 1H), 1.91 (s, 3H), 1.88-1.75 (m, 8H), 1.52-1.42 (m, 2H), 1.41-1.29 (m, 4H). |
| Example 157 | 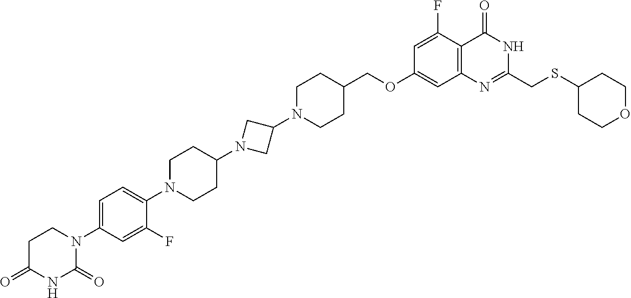 [SMILES: O=C1CCN(c2ccc(N3CCC(N4CC(N5CCC(COc6cc(F)c7c(=O)[nH]c(CSC8CCOCC8)nc7c6)CC5)C4)CC3)c(F)c2)C(=O)N1] 1-(3-fluoro-4-(4-(3-(4-(((5-fluoro-4-oxo-2- (((tetrahydro-2H-pyran-4-yl)thio)methyl)-3,4- dihydroquinazolin-7-yl)oxy)methyl)piperidin-1- yl)azetidin-1-yl)piperidin-1- yl)phenyl)dihydropyrimidine-2,4(1H,3H)-dione | LCMS (ESI, m/z): 752.35 [M + H]⁺. 1H NMR (400 MHz, DMSO-d6) δ 12.14 (br, 1H), 10.35 (s, 1H), 7.14 (d, J = 2.4 Hz, 1H), 7.08- 6.98 (m, 2H), 6.93- 6.83 (m, 2H), 3.98 (d, J = 5.8 Hz, 2H), 3.88- 3.78 (m, 2H), 3.74 (t, J = 6.7 Hz, 2H), 3.62 (s, 2H), 3.58-3.35 (m, 4H), 3.28-3.21 (m, 2H), 3.15-3.00 (m, 1H), 2.89-2.65 (m, 9H), 2.19-2.11 (m, 1H), 1.95- 1.86 (m, 2H), 1.80- 1.71 (m, 7H), 1.60- 1.40 (m, 2H), 1.30- 1.00 (m, 4H). |
| Example 158 | 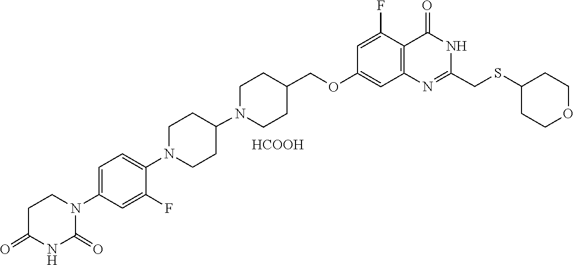 [SMILES: O=C1CCN(c2ccc(N3CCC(N4CCC(COc5cc(F)c6c(=O)[nH]c(CSC7CCOCC7)nc6c5)CC4)CC3)c(F)c2)C(=O)N1 ; "HCOOH"] 1-(3-fluoro-4-(4-(((5-fluoro-4-oxo-2-(((tetrahydro- 2H-pyran-4-yl)thio)methyl)-3,4-dihydroquinazolin-7- yl)oxy)methyl)-[1,4′-bipiperidin]-1′- yl)phenyl)dihydropyrimidine-2,4(1H,3H)-dione formate | LCMS (ESI, m/z): 697.25 [M + H]⁺. 1H NMR (400 MHz, DMSO-d6) δ 12.17 (s, 1H), 10.36 (s, 1H), 8.17 (s, 1H), 7.26-7.13 (m, 1H), 7.10-6.94 (m, 2H), 3.99 (d, J = 5.7 Hz, 2H), 3.86-3.78 (m, 2H), 3.76-3.66 (m, 2H), 3.62 (s, 2H), 3.46-3.32 (m, 4H), 3.10-3.02 (m, 1H), 2.96 (d, J = 10.5 Hz, 2H), 2.67-2.50 (m, 4H), 2.40-2.30 (m, 1H), 2.23 (t, J = 11.1 Hz, 2H), 2.03-1.71 (m, 7H), 1.66-1.56 (m, 2H), 1.52-1.37 (m, 2H), 1.35-1.21 (m, 2H). |
| Example 159 | 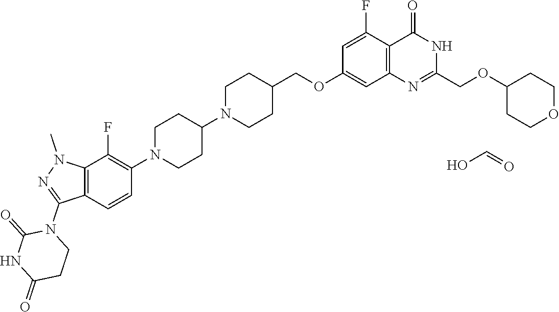 [SMILES: Cn1nc(N2CCC(=O)NC2=O)c2ccc(N3CCC(N4CCC(COc5cc(F)c6c(=O)[nH]c(COC7CCOCC7)nc6c5)CC4)CC3)c(F)c21 ; O=CO] 1-(7-fluoro-6-(4-(((5-fluoro-4-oxo-2-(((tetrahydro- 2H-pyran-4-yl)oxy)methyl)-3,4-dihydroquinazolin-7- yl)oxy)methyl)-[1,4′-bipiperidin]-1′-yl)-1-methyl-1H- indazol-3-yl)dihydropyrimidine-2,4(1H,3H)-dione formate | LCMS (ESI, m/z): 735.40 [M + H]⁺. 1H NMR (400 MHz, DMSO-d6) δ 12.06 (br, 1H), 10.58 (s, 1H), 8.32 (s, 3H), 7.34 (d, J = 8.7 Hz, 1H), 7.00-6.85 (m, 3H), 4.38 (s, 2H), 4.15- 3.95 (m, 6H), 3.92- 3.86 (m, 3H), 3.85- 3.78 (m, 3H), 3.02- 2.90 (m, 3H), 2.88- 2.65 (m, 5H), 2.24- 2.16 (m, 2H), 1.98- 1.72 (m, 7H), 1.65 (q, J = 11.5, 11.1 Hz, 2H), 1.50-1.40 (m, 2H), 1.38-1.18 (m, 3H). |
| Example 160 | 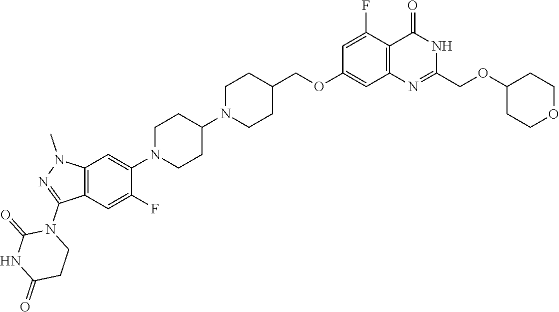 [SMILES: Cn1nc(N2CCC(=O)NC2=O)c2cc(F)c(N3CCC(N4CCC(COc5cc(F)c6c(=O)[nH]c(COC7CCOCC7)nc6c5)CC4)CC3)cc21] 1-(5-fluoro-6-(4-(((5-fluoro-4-oxo-2-(((tetrahydro- 2H-pyran-4-yl)oxy)methyl)-3,4-dihydroquinazolin-7- yl)oxy)methyl)-[1,4′-bipiperidin]-1′-yl)-1-methyl-1H- indazol-3-yl)dihydropyrimidine-2,4(1H,3H)-dione | LCMS (ESI, m/z): 735.35 [M + H]⁺. 1H NMR (400 MHz, DMSO-d6) δ 12.04 (br, 1H), 10.53 (s, 1H), 7.35 (d, J = 12.7 Hz, 1H), 7.10 (d, J = 7.1 Hz, 1H), 6.97-6.87 (m, 2H), 4.38 (s, 2H), 4.00 (d, J = 5.8 Hz, 2H), 3.94 (s, 3H), 3.90 (t, J = 6.7 Hz, 2H), 3.85-3.80 (m, 2H), 3.70-3.63 (m, 2H), 3.39-3.38 (m, 1H), 3.35-3.34 (m, 1H), 3.31-3.30 (m, 1H), 3.09-3.02 (m, 2H), 2.77-2.69 (m, 4H), 2.64-2.57 (m, 1H), 2.40-2.33 (m, 2H), 1.95-1.87 (m, 4H), 1.85-1.80 (m, 3H), 1.75-1.68 (m, 2H), 1.51-1.43 (m, 2H), 1.40-1.30 (m, 2H). |
| Example 161 | 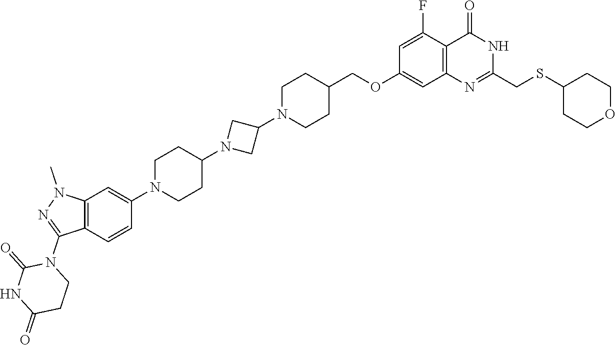 [SMILES: Cn1nc(N2CCC(=O)NC2=O)c2ccc(N3CCC(N4CC(N5CCC(COc6cc(F)c7c(=O)[nH]c(CSC8CCOCC8)nc7c6)CC5)C4)CC3)cc21] 1-(6-(4-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)azetidin-1-yl)piperidin- 1-yl)-1-methyl-1H-indazol-3-yl)dihydropyrimidine- 2,4(1H,3H)-dione | LCMS (ESI, m/z): 788.45 [M + H]⁺. 1H NMR (400 MHz, DMSO-d6) 10.49 (s, 1H), 7.43 (d, J = 9.0 Hz, 1H), 6.93-6.79 (m, 4H), 4.05-3.77 (m, 9H), 3.65 (s, 4H), 3.50- 3.45 (m, 4H), 3.15- 3.01 (m, 1H), 2.95- 2.70 (m, 9H), 2.20 (s, 1H), 1.89 (d, J = 13.3 Hz, 2H), 1.75 (d, J = 11.9 Hz, 7H), 1.52- 1.38 (m, 2H), 1.37- 1.22 (m, 4H). |
| Example 162 | 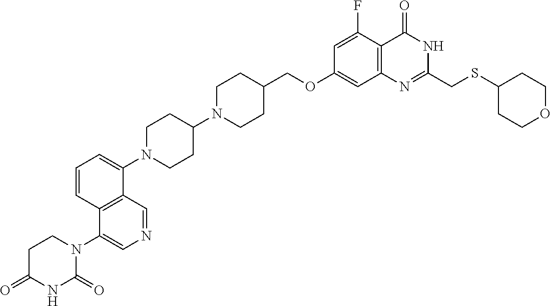 [SMILES: O=C1CCN(c2cncc3c(N4CCC(N5CCC(COc6cc(F)c7c(=O)[nH]c(CSC8CCOCC8)nc7c6)CC5)CC4)cccc23)C(=O)N1] 1-(8-(4-(((5-fluoro-4-oxo-2-(((tetrahydro-2H-pyran-4- yl)thio)methyl)-3,4-dihydroquinazolin-7- yl)oxy)methyl)-[1,4′-bipiperidin]-1′-yl)isoquinolin-4- yl)dihydropyrimidine-2,4(1H,3H)-dione | LCMS (ESI, m/z): 730.35 [M + H]⁺. 1H NMR (400 MHz, DMSO-d6) δ 12.17 (s, 1H), 10.53 (s, 1H), 9.41 (s, 1H), 8.53 (s, 1H), 7.75-7.67 (m, 1H), 7.60 (d, J = 8.4 Hz, 1H), 7.26 (d, J = 7.5 Hz, 1H), 6.95-6.86 (m, 2H), 4.07-3.96 (m, 2H), 3.95-3.86 (m, 1H), 3.85-3.79 (m, 2H), 3.75-3.66 (m, 1H), 3.66 (s, 2H), 3.53-3.40 (m, 2H), 3.38-3.35 (m, 1H), 3.31-3.30 (m, 1H), 3.11-2.91 (m, 4H), 2.90-2.70 (m, 3H), 2.30-2.20 (m, 2H), 1.96-1.74 (m, 9H), 1.53-1.40 (m, 2H), 1.40-1.28 (m, 2H), 1.28-1.25 (m, 1H). |
| Example 163 | 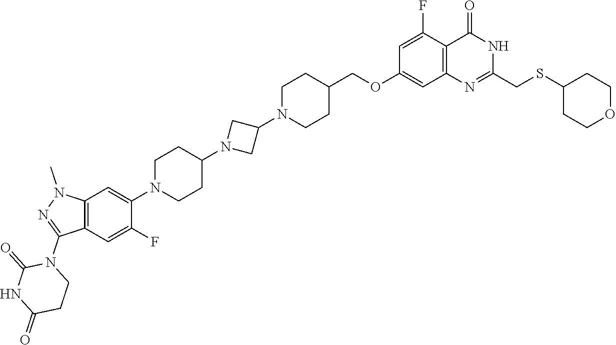 1-(5-fluoro-6-(4-(((5-fluoro-4-oxo-2-(((tetrahydro- 2H-pyran-4-yl)thio)methyl)-3,4-dihydroquinazolin-7- yl)oxy)methyl)-[1,4′-bipiperidin]-1′-yl)-1-methyl-1H- indazol-3-yl)dihydropyrimidine-2,4(1H,3H)-dione. | LCMS (ESI, m/z): 751.30 [M + H]⁺. 1H NMR (300 MHz, DMSO-d6) δ 12.16 (br, 1H), 10.50 (s, 1H), 7.34 (d, J = 12.8 Hz, 1H), 7.09 (d, J = 7.1 Hz, 1H), 7.00-6.85 (m, 2H), 4.04-3.75 (m, 9H), 3.62 (s, 2H), 3.62-3.48 (m, 3H), 3.45-3.30 (m, 5H), 3.12-2.99 (m, 2H), 2.79-2.65 (m, 4H), 1.94-1.70 (m, 8H), 1.55-1.31 (m, 4H), 1.30-1.20 (m, 1H). |
| Example 164 | 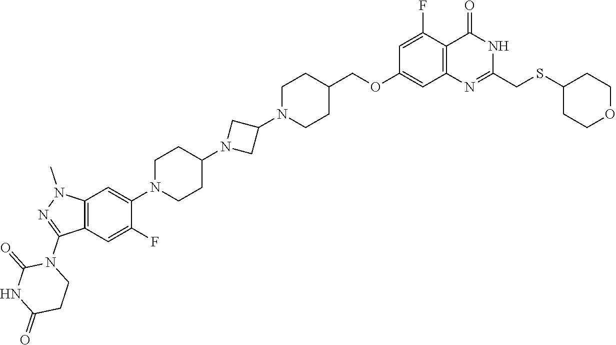 [SMILES: Cn1nc(N2CCC(=O)NC2=O)c2cc(F)c(N3CCC(N4CC(N5CCC(COc6cc(F)c7c(=O)[nH]c(CSC8CCOCC8)nc7c6)CC5)C4)CC3)cc21] 1-(5-fluoro-6-(4-(3-(4-(((5-fluoro-4-oxo-2- (((tetrahydro-2H-pyran-4-yl)thio)methyl)-3,4- dihydroquinazolin-7-yl)oxy)methyl)piperidin-1- yl)azetidin-1-yl)piperidin-1-yl)-1-methyl-1H-indazol- 3-yl)dihydropyrimidine-2,4(1H,3H)-dione | LCMS (ESI, m/z): 806.25 [M + H]⁺. 1H NMR (300 MHz, DMSO-d6) δ 12.02 (br, 1H), 10.50 (s, 1H), 7.33 (dt, J = 13.1, 2.6 Hz, 1H), 7.09 (dd, J = 7.2, 2.1 Hz, 1H), 6.93-6.82 (m, 2H), 4.02-3.80 (m, 11H), 3.63 (s, 3H), 3.15- 3.00 (m, 2H), 2.80- 2.69 (m, 10H), 2.21- 2.10 (m, 1H), 1.90- 1.70 (m, 9H), 1.54- 1.20 (m, 7H). |
| Example 165 | 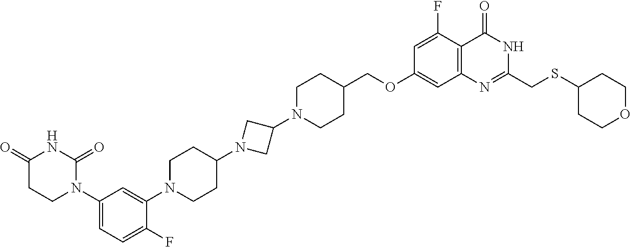 [SMILES: O=C1CCN(c2ccc(F)c(N3CCC(N4CC(N5CCC(COc6cc(F)c7c(=O)[nH]c(CSC8CCOCC8)nc7c6)CC5)C4)CC3)c2)C(=O)N1] 1-(4-fluoro-3-(4-(3-(4-(((5-fluoro-4-oxo-2- (((tetrahydro-2H-pyran-4-yl)thio)methyl)-3,4- dihydroquinazolin-7-yl)oxy)methyl)piperidin-1- yl)azetidin-1-yl)piperidin-1- yl)phenyl)dihydropyrimidine-2,4(1H,3H)-dione | LCMS (ESI, m/z): 753.25 [M + H]⁺. 1H NMR (400 MHz, DMSO-d6) δ 12.16 (s, 1H), 10.33 (s, 1H), 7.16- 7.08 (m, 1H), 6.99 (dd, J = 8.1, 2.6 Hz, 1H), 6.91-6.81 (m, 3H), 3.98 (d, J = 5.8 Hz, 2H), 3.80-3.76 (m, 2H), 3.74-3.69 (m, 2H), 3.62 (s, 2H), 3.42- 3.37 (m, 3H), 3.30- 3.24 (s, 2H), 3.10-3.02 (m, 1H), 2.82-2.66 (m, 9H), 2.19-2.95 (m, 1H), 1.94-1.85 (m, 2H), 1.81-1.71 (m, 7H), 1.51-1.40 (m, 2H), 1.39-1.23 (m, 5H). |
| Example 166 | 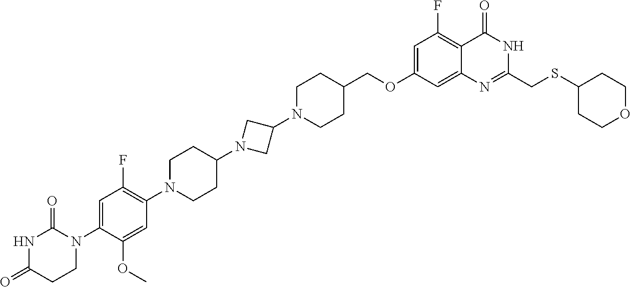 [SMILES: COc1cc(N2CCC(N3CC(N4CCC(COc5cc(F)c6c(=O)[nH]c(CSC7CCOCC7)nc6c5)CC4)C3)CC2)c(F)cc1N1CCC(=O)NC1=O] 1-(5-fluoro-4-(4-(3-(4-(((5-fluoro-4-oxo-2- (((tetrahydro-2H-pyran-4-yl)thio)methyl)-3,4- dihydroquinazolin-7-yl)oxy)methyl)piperidin-1- yl)azetidin-1-yl)piperidin-1-yl)-2- methoxyphenyl)dihydropyrimidine-2,4(1H,3H)-dione | LCMS (ESI, m/z): 782.30 [M + H]⁺. 1H NMR (300 MHz, DMSO-d6) δ 12.15 (s, 1H), 10.27 (s, 1H), 7.08 (d, J = 12.8 Hz, 1H), 6.93-6.83 (m, 2H), 6.68 (d, J = 8.1 Hz, 1H), 4.00 (d, J = 5.6 Hz, 2H), 3.89-3.78 (m, 5H), 3.63 (s, 2H), 3.54 (t, J = 6.7 Hz, 2H), 3.41-3.32 (m, 5H), 3.29-3.26 (m, 5H), 3.13-3.00 (m, 1H), 2.82-2.74 (m, 3H), 2.69-2.62 (m, 2H), 1.95-1.70 (m, 10H), 1.53-1.24 (m, 6H) |
| Example 167 | 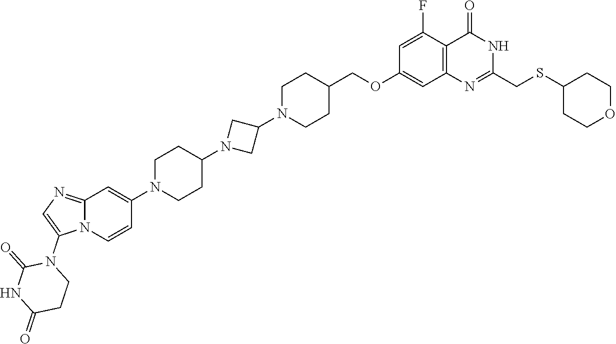 [SMILES: O=C1CCN(c2cnc3cc(N4CCC(N5CC(N6CCC(COc7cc(F)c8c(=O)[nH]c(CSC9CCOCC9)nc8c7)CC6)C5)CC4)ccn23)C(=O)N1] 1-(7-(4-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)azetidin-1-yl)piperidin- 1-yl)imidazo[1,2-a]pyridin-3-yl)dihydropyrimidine- 2,4(1H,3H)-dione. | LCMS (ESI, m/z): 774.40 [M + H]⁺. 1H NMR (400 MHz, DMSO-d6) δ 12.16 (s, 1H), 10.59 (s, 1H), 8.02 (d, J = 8.0 Hz, 1H), 7.27 (s, 1H), 6.92-6.83 (m, 3H), 6.66 (s, 1H), 3.98 (d, J = 8.0 Hz, 2H), 3.82 (d, J = 6.7 Hz, 2H), 3.75 (t, J = 11.5 Hz, 2H), 3.62 (s, 4H), 3.44-3.30 (m, 4H), 3.11-3.00 (m, 1H), 2.93-2.72 (m, 9H), 2.19-2.10 (m, 1H), 1.89 (d, J = 16.0 Hz, 2H), 1.81-1.66 (m, 7H), 1.52-1.38 (m, 2H), 1.32-1.23 (m, 4H). |
| Example 168 | 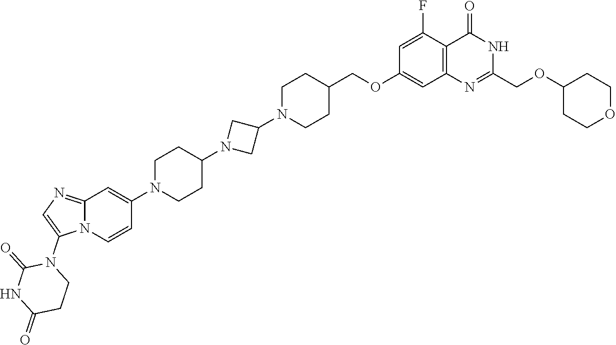 [SMILES: O=C1CCN(c2cnc3cc(N4CCC(N5CC(N6CCC(COc7cc(F)c8c(=O)[nH]c(COC9CCOCC9)nc8c7)CC6)C5)CC4)ccn23)C(=O)N1] 1-(7-(4-(3-(4-(((5-fluoro-4-oxo-2-(((tetrahydro-2H- pyran-4-yl)oxy)methyl)-3,4-dihydroquinazolin-7- yl)oxy)methyl)piperidin-1-yl)azetidin-1-yl)piperidin- 1-yl)imidazo[1,2-a]pyridin-3-yl)dihydropyrimidine- 2,4(1H,3H)-dione | LCMS (ESI, m/z): 758.40 [M + H]⁺. 1H NMR (400 MHz, DMSO-d6) δ 12.00 (s, 1H), 10.57 (s, 1H), 8.02 (d, J = 8.0 Hz, 1H), 7.26 (s, 1H), 6.95-6.82 (m, 3H), 6.65 (d, J = 4.0 Hz, 1H), 4.37 (s, 2H), 3.98 (d, J = 8.0 Hz, 2H), 3.87- 3.79 (m, 2H), 3.78- 3.72 (m, 2H), 3.70- 3.56 (m, 3H), 3.43- 3.33 (m, 4H), 2.92- 2.72 (m, 8H), 2.19- 2.11 (m, 1H), 1.94- 1.86 (m, 2H), 1.82- 1.70 (m, 7H), 1.55- 1.42 (m, 2H), 1.32- 1.22 (m, 5H). |
| Example 169 | 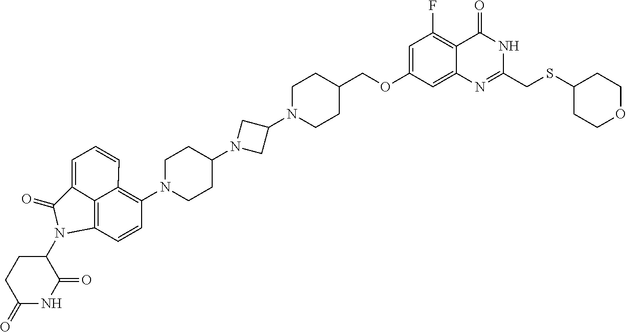 [SMILES: O=C1CCC(N2C(=O)c3cccc4c(N5CCC(N6CC(N7CCC(COc8cc(F)c9c(=O)[nH]c(CSC%10CCOCC%10)nc9c8)CC7)C6)CC5)ccc2c34)C(=O)N1] 3-(6-(4-(3-(4-(((5-fluoro-4-oxo-2-(((tetrahydro-2H- pyran-4-yl)thio)methyl)-3,4-dihydroquinazolin-7- yl)oxy)methyl)piperidin-1-yl)azetidin-1-yl)piperidin- 1-yl)-2-oxobenzo[cd]indol-1(2H)-yl)piperidine-2,6- dione | LCMS (ESI, m/z): 824.40 [M + H]⁺. 1H NMR (400 MHz, DMSO-d6) δ 12.17 (s, 1H), 11.11 (s, 1H), 8.22 (d, J = 8.2 Hz, 1H), 8.08 (d, J = 6.9 Hz, 1H), 7.86- 7.78 (m, 1H), 7.01 (d, J = 7.7 Hz, 1H), 6.95 (d, J = 7.7 Hz, 1H), 6.91- 6.84 (m, 2H), 5.42 (dd, J = 12.7, 5.4 Hz, 1H), 3.99 (d, J = 5.7 Hz, 2H), 3.87-3.78 (m, 2H), 3.62 (s, 2H), 3.50-3.41 (m, 2H), 3.39-3.34 (m, 2H), 3.31-3.28 (m, 2H), 3.11-3.03 (m, 1H), 3.00-2.91 (m, 1H), 2.88-2.67 (m, 8H), 2.63 (s, 1H), 2.21 (s, 1H), 2.13-2.06 (m, 1H), 1.97-1.83 (m, 4H), 1.83-1.63 (m, 5H), 1.61-1.40 (m, 4H), 1.37-1.24 (m, 2H). |

### Example 170: Synthesis of 3-((2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)-4-hydroxypiperidin-1-yl)phenyl)amino)piperidine-2,6-dione

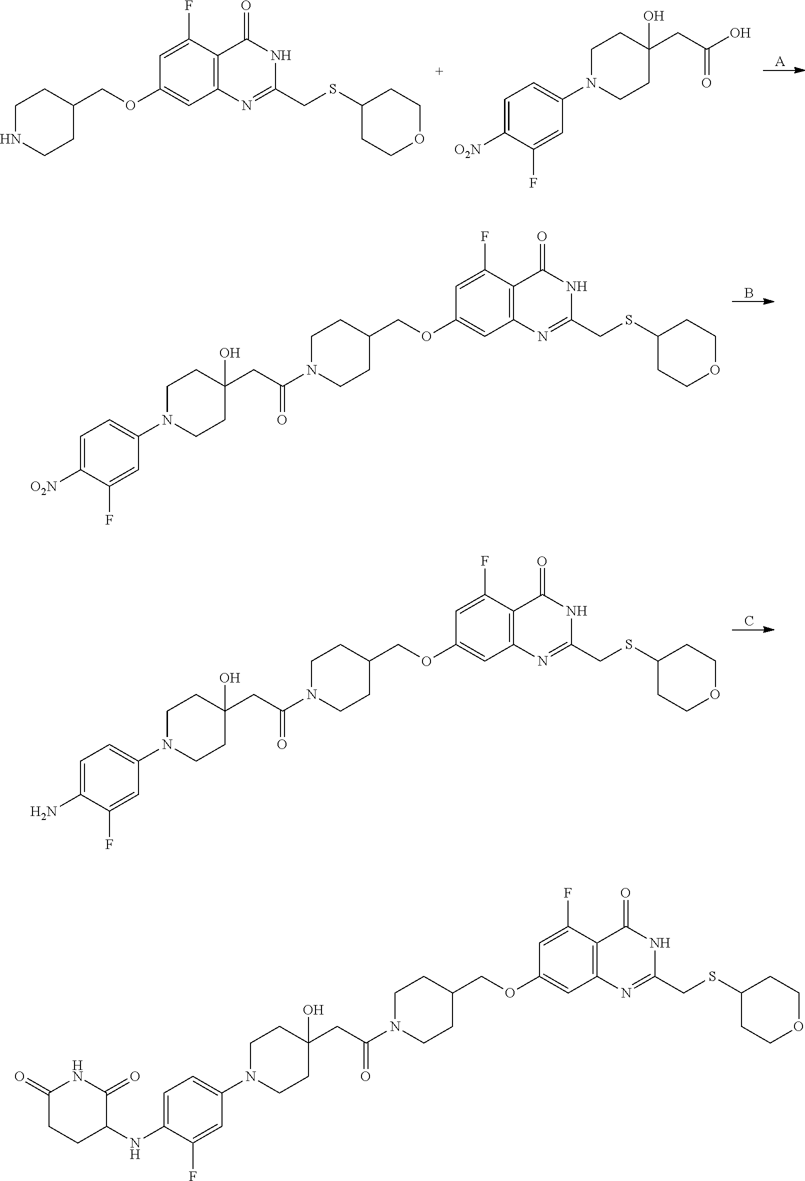

### Step A

A mixture of 2-(1-(3-fluoro-4-nitrophenyl)-4-hydroxypiperidin-4-yl)acetic acid (0.700 g, 2.35 mmol, 1 equiv), 5-fluoro-7-(piperidin-4-ylmethoxy)-2-(((tetrahydro-2H-pyran-4-yl)thio)methyl)quinazolin-4(3H)-one (1.05 g, 2.58 mmol, 1.1 equiv), HATU (1.34 g, 3.52 mmol, 1.5 equiv) and DIEA (0.910 g, 7.04 mmol, 3 equiv) in DMF (4 mL) was stirred for 2 hours. The crude product was purified by reverse phase flash chromatography (ACN:H₂O=1:1) to afford 5-fluoro-7-((1-(2-(1-(3-fluoro-4-nitrophenyl)-4-hydroxypiperidin-4-yl)acetyl)piperidin-4-yl)methoxy)-2-(((tetrahydro-2H-pyran-4-yl)thio)methyl)quinazolin-4(3H)-one (1.27 g, 79%) as a yellow solid. LCMS (ESI, m/z): 688.20 [M+H]⁺.

### Step B

A mixture of 5-fluoro-7-((1-(2-(1-(3-fluoro-4-nitrophenyl)-4-hydroxypiperidin-4-yl)acetyl)piperidin-4-yl)methoxy)-2-(((tetrahydro-2H-pyran-4-yl)thio)methyl)quinazolin-4(3H)-one (1.25 g, 1.82 mmol, 1 equiv), Fe (0.51 g, 9.1 mmol, 5 equiv) and NH₄Cl (0.29 g, 5.5 mmol, 3 equiv) in EtOH (6 mL) and water (3 mL) was stirred for 2 hours at 80° C. The mixture was concentrated and the residue was purified by silica gel column chromatography, eluting with CH₂Cl₂/MeOH (9:1) to afford 7-((1-(2-(1-(4-amino-3-fluorophenyl)-4-hydroxypiperidin-4-yl)acetyl)piperidin-4-yl)methoxy)-5-fluoro-2-(((tetrahydro-2H-pyran-4-yl)thio)methyl)quinazolin-4(3H)-one (1.05 g, 88%) as a green solid. LCMS (ESI, m/z): 658.05 [M+H]⁺.

### Step C

A mixture of 7-((1-(2-(1-(4-amino-3-fluorophenyl)-4-hydroxypiperidin-4-yl)acetyl)piperidin-4-yl)methoxy)-5-fluoro-2-(((tetrahydro-2H-pyran-4-yl)thio)methyl)quinazolin-4(3H)-one (450 mg, 0.684 mmol, 1 equiv), 3-bromopiperidine-2,6-dione (394 mg, 2.05 mmol, 3 equiv) and NaHCO₃ (287 mg, 3.42 mmol, 5 equiv) in ACN (5 mL) was stirred overnight at 90° C. The crude product was purified by reverse phase flash with the following conditions (ACN:water (10 mmol/L NH₄HCO₃)=3:2) to afford 3-((2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)-4-hydroxypiperidin-1-yl)phenyl)amino)piperidine-2,6-dione (269 mg, 51%) as a purple solid. LCMS (ESI, m/z): 769.35 [M+H]⁺. 1H NMR (300 MHz, DMSO-d6) δ 12.15 (s, 1H), 10.78 (s, 1H), 6.96-6.86 (m, 2H), 6.75 (t, J=9.4 Hz, 2H), 6.63 (s, 1H), 5.10-4.85 (m, 2H), 4.48 (d, J=12.8 Hz, 1H), 4.25 (d, J=7.8 Hz, 1H), 4.09-3.93 (m, 3H), 3.92-3.76 (m, 2H), 3.62 (s, 2H), 3.43-3.33 (m, 5H), 3.23-3.18 (m, 2H), 3.19-2.95 (m, 3H), 2.85-2.78 (m, 1H), 2.77-2.50 (m, 2H), 2.25-2.03 (m, 2H), 1.97-1.85 (m, 5H), 1.82-1.54 (m, 4H), 1.52-1.40 (m, 2H), 1.31-1.11 (m, 2H).

Example 171 was synthesized according to the procedure described for the synthesis of 3-((2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)-4-hydroxypiperidin-1-yl)phenyl)amino)piperidine-2,6-dione Example 170 using appropriate building blocks and modified reaction conditions (such as reagents, reagent ratio, temperature, and reaction time) and purification conditions as needed.

| Example | Structure | Analytical Data |
|---|---|---|
| Example 171 | 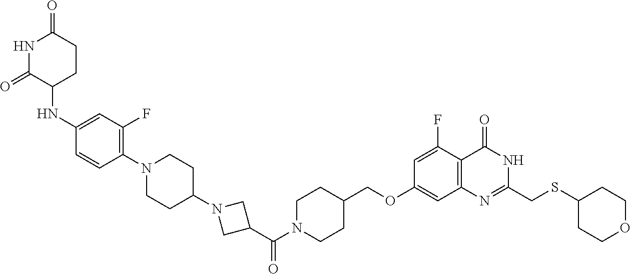 [SMILES: O=C1CCC(Nc2ccc(N3CCC(N4CC(C(=O)N5CCC(COc6cc(F)c7c(=O)[nH]c(CSC8CCOCC8)nc7c6)CC5)C4)CC3)c(F)c2)C(=O)N1] 3-((3-fluoro-4-(4-(3-(4-(((5-fluoro-4-oxo-2- (((tetrahydro-2H-pyran-4-yl)thio)methyl)-3,4- dihydroquinazolin-7-yl)oxy)methyl)piperidine-1- carbonyl)azetidin-1-yl)piperidin-1- yl)phenyl)amino)piperidine-2,6-dione | LCMS (ESI, m/z): 794.40 [M + H]⁺. 1H NMR (400 MHz, DMSO-d6) δ 12.18 (br, 1H), 10.78 (s, 1H), 8.21 (s, 1H), 6.95-6.76 (m, 3H), 6.49 (dd, J = 14.9, 2.6 Hz, 1H), 6.41 (dd, J = 8.7, 2.6 Hz, 1H), 5.79 (d, J = 7.6 Hz, 1H), 4.39 (d, J = 12.8 Hz, 1H), 4.30-4.20 (m, 1H), 3.99 (d, J = 6.5 Hz, 2H), 3.84-3.79 (m, 2H), 3.62 (s, 3H), 3.44 (d, J = 7.0 Hz, 3H), 3.38-3.27 (m, 2H), 3.15 (d, J = 4.2 Hz, 2H), 3.11-2.90 (m, 4H), 2.80-2.67 (m, 1H), 2.64-2.53 (m, 4H), 2.15-1.99 (m, 3H), 1.89-1.64 (m, 7H), 1.52-1.38 (m, 2H), 1.34-1.23 (m, 2H), 1.20-1.06 (m, 2H). |

### Example 172: Synthesis of 3-((3-fluoro-4-(4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidine]-1′-carbonyl)piperidin-1-yl)phenyl)amino)piperidine-2,6-dione

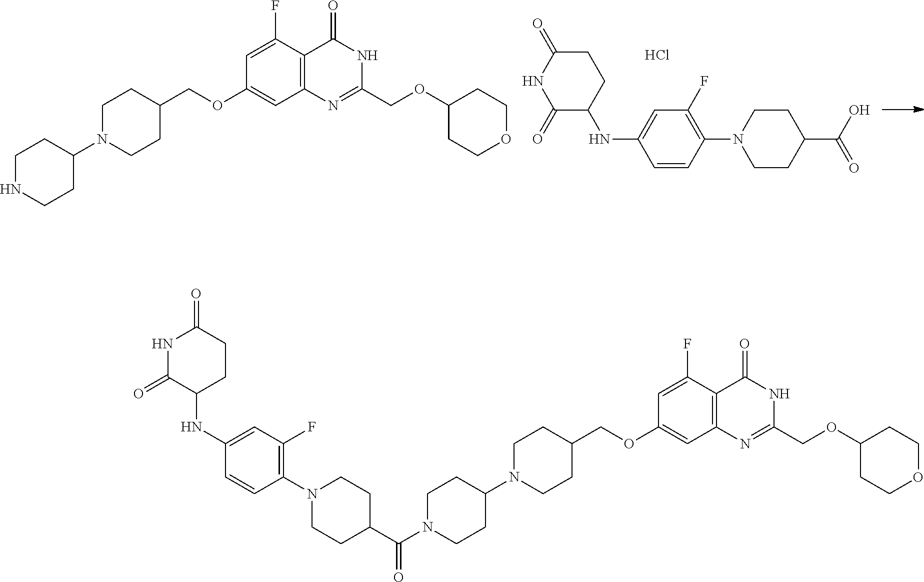

A solution of 7-([1,4′-bipiperidin]-4-ylmethoxy)-5-fluoro-2-(((tetrahydro-2H-pyran-4-yl)oxy)methyl)quinazolin-4(3H)-one (0.200 g, 0.421 mmol, 1 equiv), 1-(4-((2,6-dioxopiperidin-3-yl)amino)-2-fluorophenyl)piperidine-4-carboxylic acid hydrochloride (163 mg, 0.421 mmol, 1 equiv), HOBT (85 mg, 0.63 mmol, 1.5 equiv), EDCI (121 mg, 0.631 mmol, 1.5 equiv) and DIEA (163 mg, 1.26 mmol, 3 equiv) in DMF (4 mL) was stirred for 2 hours. The residue was purified by reverse phase flash chromatography with the following conditions: (column, C18 silica gel; mobile phase, MeCN in water (10 mmol/L NH₄HCO₃), 10% to 50% gradient in 10 min; detector, UV 254 nm). The product was repurified by prep-HPLC with the following conditions (Mobile Phase A: water (50 mmol/L NH₄HCO₃), Mobile Phase B: ACN; Flow rate: 20 mL/min; Gradient: 23% B to 33% B in 10 min; Wavelength: 254 nm/220 nm; RT (min): 16.08) to afford 3-((3-fluoro-4-(4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidine]-1′-carbonyl)piperidin-1-yl)phenyl)amino)piperidine-2,6-dione (58 mg, 17%) as a white solid. LCMS (ESI, m/z): 806.40 [M+H]⁺. 1H NMR (300 MHz, DMSO-d6) δ 11.97 (s, 1H), 10.76 (s, 1H), 6.92 (s, 1H), 6.90-6.77 (m, 2H), 6.51 (d, J=15.0 Hz, 1H), 6.43 (d, J=8.9 Hz, 1H), 5.78 (d, J=7.7 Hz, 1H), 4.51-4.45 (m, 1H), 4.38 (s, 2H), 4.29-4.19 (m, 1H), 4.05-3.95 (m, 3H), 3.89-3.76 (m, 2H), 3.74-3.67 (m, 1H), 3.40-3.30 (m, 2H), 3.19-3.12 (m, 2H), 3.10-2.94 (m, 1H), 2.88-2.81 (m, 2H), 2.74-2.61 (m, 6H), 2.49-2.41 (m, 1H), 2.21-2.05 (m, 3H), 1.94-1.83 (m, 3H), 1.80-1.65 (m, 9H), 1.55-1.40 (m, 2H), 1.39-1.15 (m, 4H).

Examples 173-178 were synthesized according to the procedure described for the synthesis of 3-((3-fluoro-4-(4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidine]-1′-carbonyl)piperidin-1-yl)phenyl)amino)piperidine-2,6-dione Example 172 using appropriate building blocks and modified reaction conditions (such as reagents, reagent ratio, temperature, and reaction time) and purification conditions as needed.

| Example | Structure | Analytical Data |
|---|---|---|
| Example 173 | 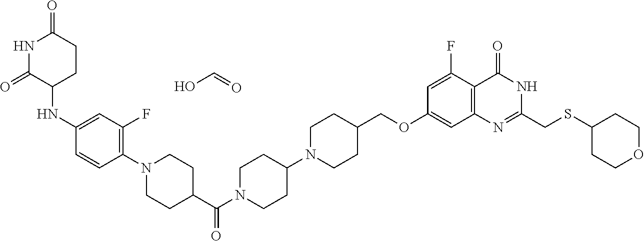 [SMILES: O=C1CCC(Nc2ccc(N3CCC(C(=O)N4CCC(N5CCC(COc6cc(F)c7c(=O)[nH]c(CSC8CCOCC8)nc7c6)CC5)CC4)CC3)c(F)c2)C(=O)N1 ; O=CO] 3-((3-fluoro-4-(4-(4-(((5-fluoro-4-oxo-2- (((tetrahydro-2H-pyran-4-yl)thio)methyl)-3,4- dihydroquinazolin-7-yl)oxy)methyl)-[1,4′- bipiperidine]-1′-carbonyl)piperidin-1- yl)phenyl)amino)piperidine-2,6-dione formate | LCMS (ESI, m/z): 822.30 [M + H]⁺. 1H NMR (400 MHz, DMSO-d6) δ 12.16 (br, 1H), 10.76 (s, 1H), 8.21 (s, 1H), 6.91-6.78 (m, 3H), 6.51 (dd, J = 14.9, 2.5 Hz, 1H), 6.42 (dd, J = 8.7, 2.6 Hz, 1H), 5.78 (d, J = 7.6 Hz, 1H), 4.51- 4.42 (m, 1H), 4.39- 4.29 (m, 1H), 4.03 (s, 2H), 3.89-3.80 (m, 3H), 3.70-3.60 (m, 2H), 3.38-3.32 (m, 2H), 3.21-2.89 (m, 6H), 2.80-2.69 (m, 6H), 2.31-2.19 (m, 2H), 2.18-2.09 (m, 1H), 1.95-1.65 (m, 12H), 1.55-1.21 (m, 6H). |
| Example 174 | 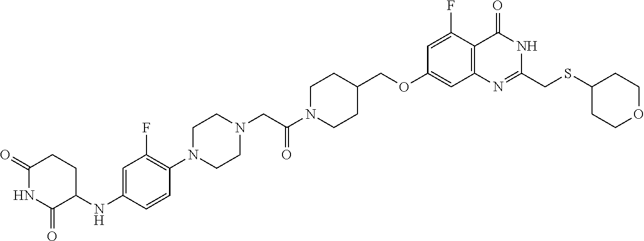 [SMILES: O=C1CCC(Nc2ccc(N3CCN(CC(=O)N4CCC(COc5cc(F)c6c(=O)[nH]c(CSC7CCOCC7)nc6c5)CC4)CC3)c(F)c2)C(=O)N1] 3-((3-fluoro-4-(4-(2-(4-(((5-fluoro-4-oxo-2- (((tetrahydro-2H-pyran-4-yl)thio)methyl)-3,4- dihydroquinazolin-7-yl)oxy)methyl)piperidin-1-yl)- 2-oxoethyl)piperazin-1-yl)phenyl)amino)piperidine- 2,6-dione | LCMS (ESI, m/z): 754.30 [M + H]⁺. 1H NMR (300 MHz, DMSO-d6) δ 12.15 (s, 1H), 10.76 (s, 1H), 6.94- 6.77 (m, 3H), 6.52 (dd, J = 15.0, 2.6 Hz, 1H), 6.42 (dd, J = 8.8, 2.6 Hz, 1H), 5.80 (d, J = 7.6 Hz, 1H), 4.40 (d, J = 12.8 Hz, 1H), 4.35- 4.21 (m, 1H), 4.19- 4.09 (m, 3H), 3.80 (d, J = 6.2 Hz, 2H), 3.62 (s, 2H), 3.40-3.30 (m, 3H), 3.15-2.91 (m, 3H), 2.89-2.81 (m, 4H), 2.83-2.59 (m, 7H), 2.15-2.04 (m, 2H), 1.95-1.79 (m, 5H), 1.58-1.38 (m, 2H), 1.36-1.05 (m, 2H). |
| Example 175 | 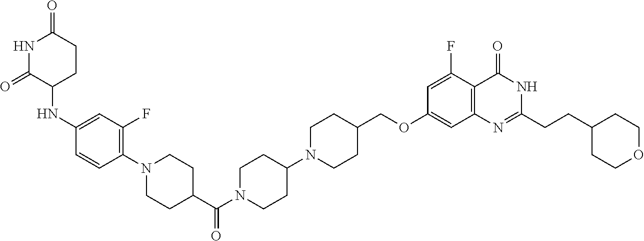 [SMILES: O=C1CCC(Nc2ccc(N3CCC(C(=O)N4CCC(N5CCC(COc6cc(F)c7c(=O)[nH]c(CCC8CCOCC8)nc7c6)CC5)CC4)CC3)c(F)c2)C(=O)N1] 3-((3-fluoro-4-(4-(4-(((5-fluoro-4-oxo-2-(2- (tetrahydro-2H-pyran-4-yl)ethyl)-3,4- dihydroquinazolin-7-yl)oxy)methyl)-[1,4′- bipiperidine]-1′-carbonyl)piperidin-1- yl)phenyl)amino)piperidine-2,6-dione | LCMS (ESI, m/z): 804.40 [M + H]⁺.1H NMR (400 MHz, DMSO-d6) δ 12.04 (s, 1H), 10.77 (s, 1H), 6.89- 6.78 (m, 3H), 6.51 (dd, J = 15.0, 2.5 Hz, 1H), 6.42 (dd, J = 8.6, 2.6 Hz, 1H), 5.80 (d, J = 7.7 Hz, 1H), 4.50-4.40 (m, 1H), 4.30-4.21 (m, 1H), 4.06-3.95 (m, 3H), 3.84 (dd, J = 5.8 Hz, 2H), 3.30-3.25 (m, 3H), 3.20-3.10 (m, 2H), 3.05-2.95 (m, 1H), 2.89-2.81 (m, 2H), 2.80- 2.53 (m, 7H), 2.25- 2.05 (m, 3H), 1.90-1.43 (m, 13H), 1.42-1.35 (m, 1H), 1.34-1.10 (m, 7H). |
| Example 176 | 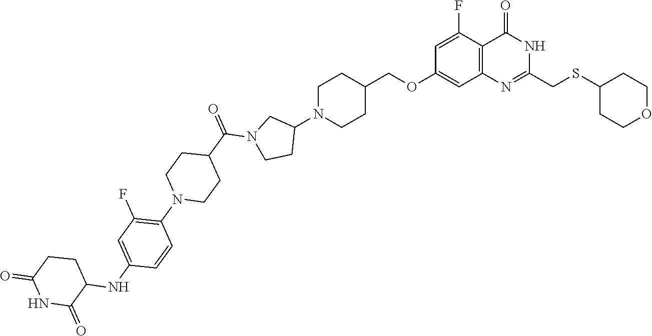 [SMILES: O=C1CCC(Nc2ccc(N3CCC(C(=O)N4CCC(N5CCC(COc6cc(F)c7c(=O)[nH]c(CSC8CCOCC8)nc7c6)CC5)C4)CC3)c(F)c2)C(=O)N1] 3-((3-fluoro-4-(4-(3-(4-(((5-fluoro-4-oxo-2- (((tetrahydro-2H-pyran-4-yl)thio)methyl)-3,4- dihydroquinazolin-7-yl)oxy)methyl)piperidin-1- yl)pyrrolidine-1-carbonyl)piperidin-1- yl)phenyl)amino)piperidine-2,6-dione | LCMS (ESI, m/z): 808.30 [M + H]⁺. 1H NMR (300 MHz, DMSO-d6) δ 12.13 (s, 1H), 10.76 (s, 1H), 6.99- 6.73 (m, 3H), 6.61- 6.22 (m, 2H), 5.78 (d, J = 9.0 Hz, 1H), 4.36- 4.16 (m, 1H), 4.04- 3.94 (m, 2H), 3.90- 3.78 (m, 2H), 3.73- 3.60 (m, 3H), 3.58- 3.42 (m, 1H), 3.39- 3.31 (m, 4H), 3.25- 3.13 (m, 3H),3.13- 2.80 (m, 4H),2.80- 2.58 (m, 4H), 2.16- 1.95 (m, 4H), 1.95-1.58 (m, 11H), 1.57-1.19 (m, 4H). |
| Example 177 | 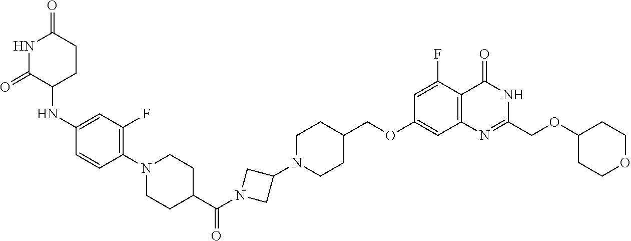 [SMILES: O=C1CCC(Nc2ccc(N3CCC(C(=O)N4CC(N5CCC(COc6cc(F)c7c(=O)[nH]c(COC8CCOCC8)nc7c6)CC5)C4)CC3)c(F)c2)C(=O)N1] 3-((3-fluoro-4-(4-(3-(4-(((5-fluoro-4-oxo-2- (((tetrahydro-2H-pyran-4-yl)oxy)methyl)-3,4- dihydroquinazolin-7-yl)oxy)methyl)piperidin-1- yl)azetidine-1-carbonyl)piperidin-1- yl)phenyl)amino)piperidine-2,6-dione | LCMS (ESI, m/z): 778.25 [M + H]⁺. 1H NMR (400 MHz, DMSO-d6) δ 12.00 (br, 1H), 10.76 (s, 1H), 6.96- 6.87 (m, 2H), 6.83 (t, J = 14.0 Hz, 1H), 6.50 (dd, J = 16.0, 4.0 Hz, 1H), 6.42 (dd, J = 8.7, 2.6 Hz, 1H), 5.79 (d, J = 8.0 Hz, 1H), 4.38 (s, 2H), 4.31-4.16 (m, 2H), 4.00 (d, J = 8.0 Hz, 3H), 3.90-3.78 (m, 3H), 3.71-3.61 (m, 2H), 3.35-3.31 (m, 1H), 3.17-3.03 (m, 3H), 2.88-2.79 (m, 2H), 2.78-2.66 (m, 2H), 2.63-2.54 (m, 3H), 2.35-2.25 (m, 1H), 2.14-2.05 (m, 1H), 1.95-1.74 (m, 8H), 1.72-1.61 (m, 4H), 1.55-1.42 (m, 2H), 1.40-1.22 (m, 2H). |
| Example 178 | 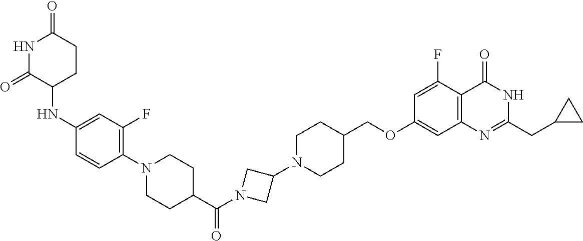 [SMILES: O=C1CCC(Nc2ccc(N3CCC(C(=O)N4CC(N5CCC(COc6cc(F)c7c(=O)[nH]c(CC8CC8)nc7c6)CC5)C4)CC3)c(F)c2)C(=O)N1] 3-((4-(4-(3-(4-(((2-(cyclopropylmethyl)-5-fluoro-4- oxo-3,4-dihydroquinazolin-7- yl)oxy)methyl)piperidin-1-yl)azetidine-1- carbonyl)piperidin-1-yl)-3- fluorophenyl)amino)piperidine-2,6-dione | LCMS (ESI, m/z): 718.30 [M + H]⁺. 1H NMR (400 MHz, DMSO-d6) δ 12.02 (s, 1H), 10.77 (s, 1H), 6.92- 6.78 (m, 3H), 6.51 (dd, J = 14.7, 2.4 Hz, 1H), 6.42 (dd, J = 8.7, 2.6 Hz, 1H), 5.80 (d, J = 7.6 Hz, 1H), 4.31-4.16 (m, 2H), 4.00 (d, J = 5.7 Hz, 3H), 3.90-3.81 (m, 1H), 3.65 (dd, J = 9.8, 5.1 Hz, 1H), 2.87- 2.77 (m, 2H), 2.76- 2.69 (m, 1H), 2.62- 2.54 (m, 3H), 2.53-2.40 (m, 2H), 2.32-2.25 (m, 1H), 2.14-2.05 (m, 1H), 1.94-1.72 (m, 6H), 1.72-1.57 (m, 4H), 1.38-1.22 (m, 2H), 1.22-1.09 (m, 1H), 0.53-0.44 (m, 2H), 0.31-0.22 (m, 2H). |

### Example 179: Synthesis of 3-((3-fluoro-4-(4-(3-(2-(4-(((5-fluoro-4-oxo-2-(((tetrahydro-2H-pyran-4-yl)thio)methyl)-3,4-dihydroquinazolin-7-yl)oxy)methyl)piperidin-l-yl)-2-oxoethyl)azetidin-1l-yl)piperidin-1l-yl)phenyl)amino)piperidine-2,6-dione

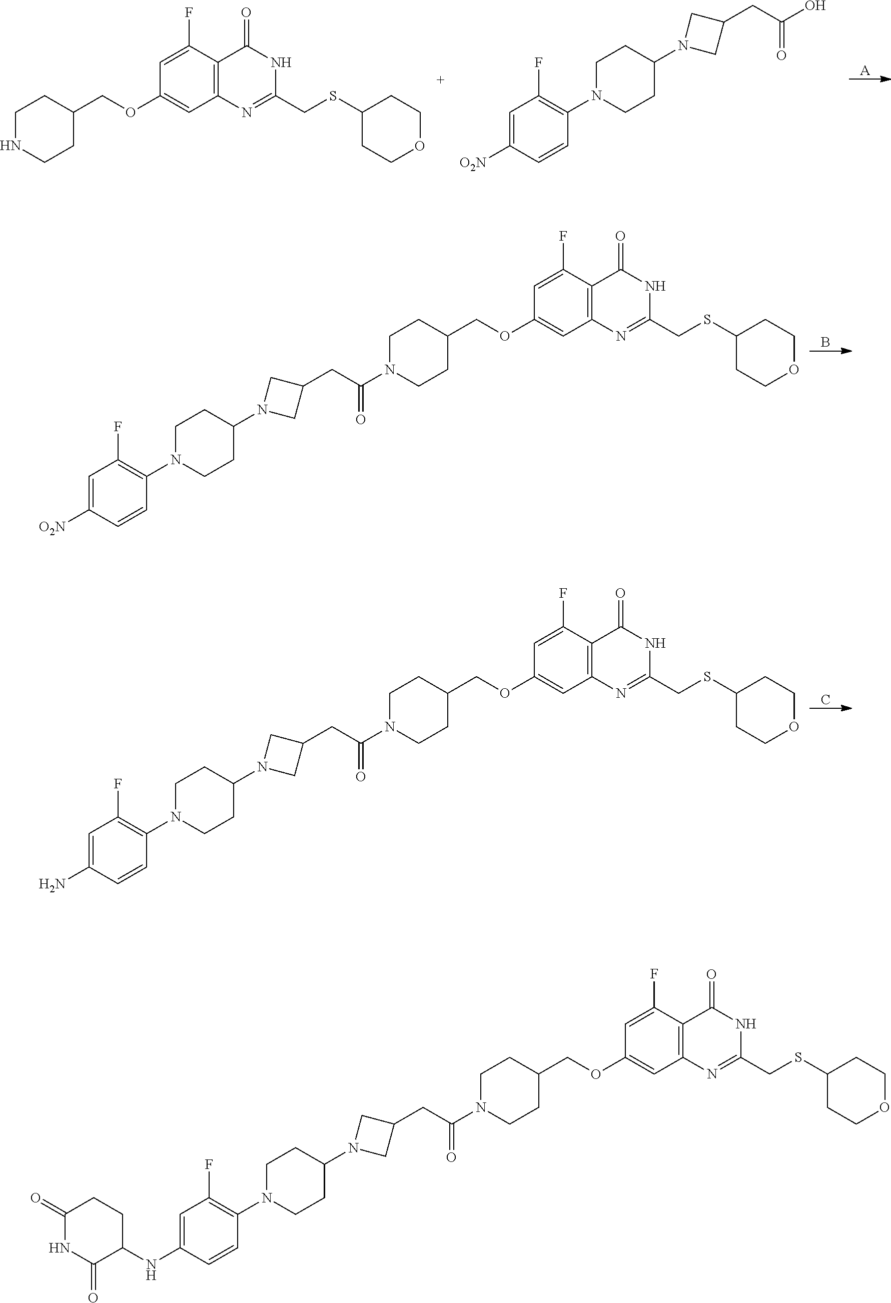

### Step A

A mixture of 5-fluoro-7-(piperidin-4-ylmethoxy)-2-(((tetrahydro-2H-pyran-4-yl)thio)methyl)quinazolin-4(3H)-one (1.0 g, 2.5 mmol, 1.0 equiv), 2-(1-(1-(2-fluoro-4-nitrophenyl)piperidin-4-yl)azetidin-3-yl)acetic acid (1.24 g, 3.68 mmol, 1.5 equiv), NMI (0.81 g, 9.8 mmol, 4.0 equiv) and TCFH (0.96 g, 3.43 mmol, 1.4 equiv) in MeCN (20 mL) was stirred for 2 hours at room temperature. After concentration, residue was purified by reverse-phase flash chromatography with the following conditions: column, C18 silica gel; mobile phase, MeCN in water (0.1% TFA), 60% to 70% gradient; detector, UV 254 nm to give 5-fluoro-7-((1-(2-(1-(1-(2-fluoro-4-nitrophenyl)piperidin-4-yl)azetidin-3-yl)acetyl)piperidin-4-yl)methoxy)-2-(((tetrahydro-2H-pyran-4-yl)thio)methyl)quinazolin-4(3H)-one (900 mg, 50%) as a yellow solid. LCMS (ESI, m/z): 727.30 [M+H]⁺.

### Step B

A mixture of 5-fluoro-7-((1-(2-(1-(1-(2-fluoro-4-nitrophenyl)piperidin-4-yl)azetidin-3-yl)acetyl)piperidin-4-yl)methoxy)-2-(((tetrahydro-2H-pyran-4-yl)thio)methyl)quinazolin-4(3H)-one (860 mg, 1.18 mmol, 1 equiv) and 10% Pd/C (1.26 g) in EtOH (5 mL) was stirred for 2 hours under 1 atm hydrogen. The mixture was filtered and washed with MeOH (3×3 mL). The filtrate was concentrated and the residue was purified by reverse-phase flash chromatography with the following conditions: column, C18 silica gel; mobile phase, MeCN in water (0.1% TFA), 60% to 66% gradient; detector, UV 254 nm to give 7-((1-(2-(1-(1-(4-amino-2-fluorophenyl)piperidin-4-yl)azetidin-3-yl)acetyl)piperidin-4-yl)methoxy)-5-fluoro-2-(((tetrahydro-2H-pyran-4-yl)thio)methyl)quinazolin-4(3H)-one (550 mg, 66%) as a light-brown solid. LCMS (ESI, m/z): 697.30 [M+H]⁺.

### Step C

A mixture of 7-((1-(2-(1-(1-(4-amino-2-fluorophenyl)piperidin-4-yl)azetidin-3-yl)acetyl)piperidin-4-yl)methoxy)-5-fluoro-2-(((tetrahydro-2H-pyran-4-yl)thio)methyl)quinazolin-4(3H)-one (290 mg, 0.416 mmol, 1 equiv), 3-bromopiperidine-2,6-dione (239 mg, 1.24 mmol, 3 equiv) and NaHCO₃ (175 mg, 2.08 mmol, 5 equiv) in MeCN (5 mL) was stirred for 1 day at 90° C. After concentration, the residue was purified by silica gel column chromatography, eluting with CH₂Cl₂/MeOH (4:1) to afford impure product. Repurification by prep-HPLC with the following conditions: (Mobile Phase A: Water (10 mmol/L NH₄HCO₃), Mobile Phase B: ACN; Flow rate: 60 mL/min; Gradient: 20% B to 40% B; Wavelength: 254/220 nm; RT (min): 9.2) to afford 3-((3-fluoro-4-(4-(3-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)azetidin-1-yl)piperidin-1-yl)phenyl)amino)piperidine-2,6-dione (8 mg, 2%) as a white solid. LCMS (ESI, m/z): 808.30 [M+H]⁺. 1H NMR (300 MHz, DMSO-d6) δ 10.75 (br, 1H), 6.85 (t, J=9.3 Hz, 1H), 6.71 (d, J=4.9 Hz, 1H), 6.60-6.46 (m, 2H), 6.46-6.38 (m, 1H), 5.83 (d, J=7.7 Hz, 1H), 5.05 (br, 1H), 4.30-4.20 (m, 1H), 3.98 (d, J=6.3 Hz, 2H), 3.91-3.76 (m, 6H), 3.65-3.50 (m, 6H), 3.20-3.05 (m, 4H), 2.90-2.70 (m, 5H), 2.65-2.60 (m, 2H), 2.30-1.80 (m, 12H), 1.60-1.40 (m, 4H), 1.24 (s, 1H).

### Example 180: Synthesis of 1-(3-fluoro-4-(4-((4-(((5-fluoro-4-oxo-2-(((tetrahydro-2H-pyran-4-yl)oxy)methyl)-3,4-dihydroquinazolin-7-yl)oxy)methyl)piperidin-1-yl)methyl)piperidin-1-yl)phenyl)dihydropyrimidine-2,4(1H,3H)-dione

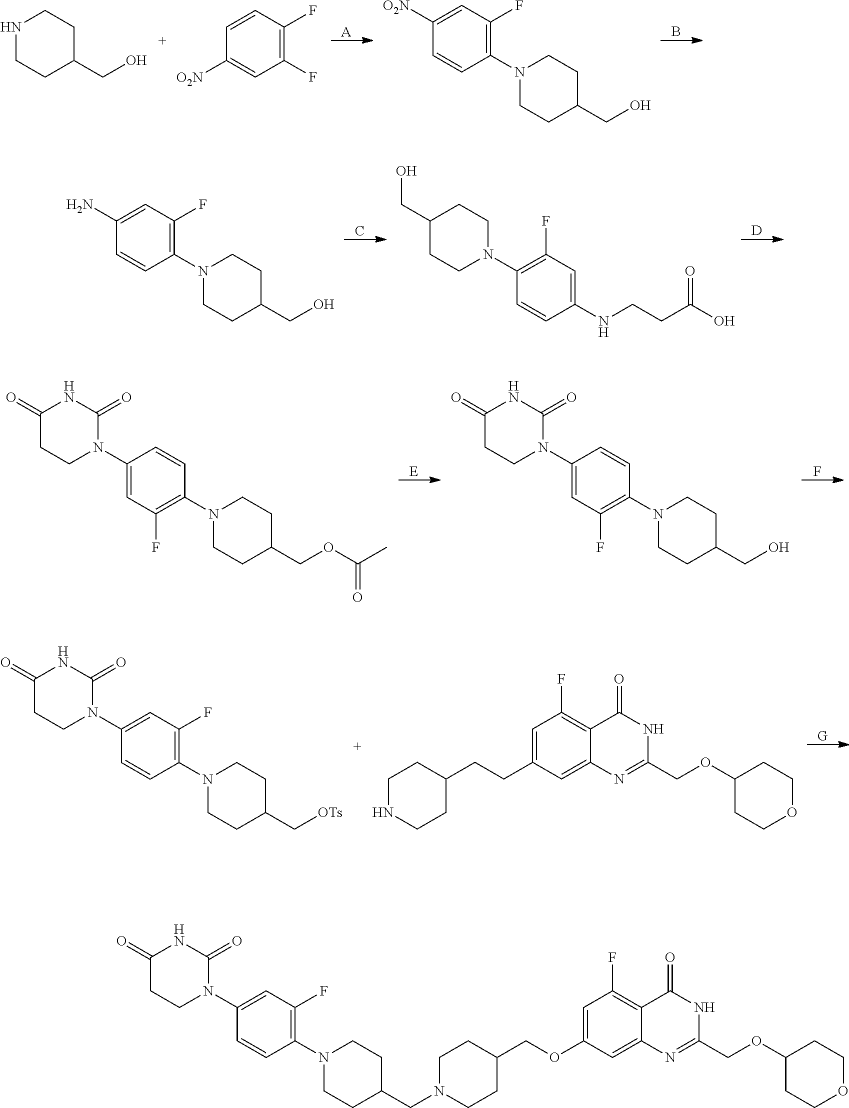

### Step A

A solution of piperidin-4-ylmethanol (7.6 g, 66.0 mmol, 1.05 equiv), 1,2-difluoro-4-nitrobenzene (10 g, 63 mmol, 1.0 equiv) and K₂CO₃ (13.0 g, 94.3 mmol, 1.5 equiv) in DMF (100 mL) was stirred for 2 hours. 100 mL water was added, and then the mixture was filtered and the filter cake was washed with water (3×20 mL) to give (1-(2-fluoro-4-nitrophenyl)piperidin-4-yl)methanol (15.4 g, 10%) as a yellow solid. LCMS (ESI, m/z): 255.10 [M+H]⁺.

### Step B

A solution of (1-(2-fluoro-4-nitrophenyl)piperidin-4-yl)methanol (15 g, 59 mmol, 1 equiv) and 10% Pd/C (12.6 g) in MeOH (200 mL) was stirred overnight under hydrogen atmosphere. The mixture was filtered and the filter cake was washed with MeOH (3×10 mL). The filtrate was concentrated under reduced pressure to give (1-(4-amino-2-fluorophenyl)piperidin-4-yl)methanol (13.6 g) as a pink solid. The product was used in the next step without further purification. LCMS (ESI, m/z): 225.10 [M+H]⁺.

### Step C

A solution of (1-(4-amino-2-fluorophenyl)piperidin-4-yl)methanol (10.0 g, 44.6 mmol, 1 equiv) and acrylic acid (9.64 g, 134 mmol, 3 equiv) in toluene (50 mL) was stirred overnight at 100° C. The precipitated solids were collected by filtration and washed with PE (3×10 mL) to give 3-((3-fluoro-4-(4-(hydroxymethyl)piperidin-1-yl)phenyl)amino)propanoic acid (2.4 g, 18%) as a brown solid. LCMS (ESI, m/z): 297.10 [M+H]⁺.

### Step D

A solution of 3-((3-fluoro-4-(4-(hydroxymethyl)piperidin-1-yl)phenyl)amino)propanoic acid (2.4 g, 8.1 mmol, 1 equiv) and urea (2.43 g, 40.5 mmol, 5 equiv) in HOAc (50 mL) was stirred overnight at 120° C. The mixture was concentrated under reduced pressure and purified by silica gel column chromatography, eluting with CH₂Cl₂/MeOH (8:1) to afford (1-(4-(2,4-dioxotetrahydropyrimidin-1(2H)-yl)-2-fluorophenyl)piperidin-4-yl)methyl acetate (1.3 g, 44%) as a white solid. LCMS (ESI, m/z): 364.10[M+H]⁺.

### Step E

A solution of (1-(4-(2,4-dioxotetrahydropyrimidin-1(2H)-yl)-2-fluorophenyl)piperidin-4-yl)methyl acetate (1.3 g, 3.6 mmol, 1 equiv) and acetyl chloride (0.34 g, 4.3 mmol, 1.2 equiv) in MeOH (30 mL) was stirred overnight under a nitrogen atmosphere. The mixture was concentrated and the residue was then dissolved in water (5 mL) and the pH was adjusted to 7 with saturated aqueous NaHCO₃. The precipitated solids were collected by filtration and washed with DCM (3×10 mL) to give 1-(3-fluoro-4-(4-(hydroxymethyl)piperidin-1-yl)phenyl)dihydropyrimidine-2,4(1H,3H)-dione (360 mg, 31%) as a white solid. LCMS (ESI, m/z): 322.10 [M+H]⁺.

### Step F

A solution of 1-(3-fluoro-4-(4-(hydroxymethyl)piperidin-1-yl)phenyl)dihydropyrimidine-2,4(1H,3H)-dione (320 mg, 0.996 mmol, 1 equiv), TsCl (228 mg, 1.20 mmol, 1.2 equiv), TEA (302 mg, 2.99 mmol, 3 equiv) and DMAP (12 mg, 0.10 mmol, 0.1 equiv) in DCM (10 mL) was stirred for 3 hours. The mixture was concentrated and the residue was purified by silica gel column chromatography, eluting with CH₂Cl₂/MeOH (8:1) to afford (1-(4-(2,4-dioxotetrahydropyrimidin-1(2H)-yl)-2-fluorophenyl)piperidin-4-yl)methyl 4-methylbenzenesulfonate (260 mg, 55%) as a yellow solid. LCMS (ESI, m/z): 476.10 [M+H]⁺.

### Step G

A solution of (1-(4-(2,4-dioxotetrahydropyrimidin-1(2H)-yl)-2-fluorophenyl)piperidin-4-yl)methyl 4-methylbenzenesulfonate (200 mg, 0.42 mmol, 1 equiv), 5-fluoro-7-(piperidin-4-ylmethoxy)-2-(((tetrahydro-2H-pyran-4-yl)oxy)methyl)quinazolin-4(3H)-one (165 mg, 0.42 mmol, 1 equiv), K₂CO₃ (174 mg, 1.26 mmol, 3 equiv) and KI (7 mg, 0.04 mmol, 0.1 equiv) in ACN (10 mL) was stirred overnight at 60° C. The mixture was concentrated, and the residue was purified by reverse-phase flash chromatography with the following conditions: column, C18 silica gel; mobile phase, MeCN in water (10 mmol/L NH₄HCO₃), 10% to 50% gradient in 10 min; detector, UV 254 nm. The crude product was purified by Prep-HPLC with the following conditions (Column: XBridge Prep Phenyl OBD Column: 19*250 mm; Mobile Phase A: Water (10 mmol/L NH₄HCO₃), Mobile Phase B: ACN; Flow rate: 25 mL/min; Gradient: 28% B to 38% B in 10 min; Wavelength: 254/220 nm; RT (min): 14.2) to afford 1-(3-fluoro-4-(4-((4-(((5-fluoro-4-oxo-2-(((tetrahydro-2H-pyran-4-yl)oxy)methyl)-3,4-dihydroquinazolin-7-yl)oxy)methyl)piperidin-1-yl)methyl)piperidin-1-yl)phenyl)dihydropyrimidine-2,4(1H,3H)-dione (17 mg, 6%) as a white solid. LCMS (ESI, m/z): 695.40 [M+H]⁺. 1H NMR (400 MHz, DMSO-d₆) δ 11.97 (br, 1H), 10.30 (s, 1H), 7.09 (dd, J=15.0, 2.5 Hz, 1H), 7.00-6.86 (m, 3H), 6.73-6.64 (m, 1H), 4.37 (s, 2H), 3.98 (d, J=5.9 Hz, 2H), 3.87-3.78 (m, 2H), 3.73-3.61 (m, 3H), 3.49-3.44 (m, 1H), 3.42-3.37 (m, 2H), 3.04-2.96 (m, 1H), 2.95-2.88 (m, 2H), 2.72-2.66 (m, 2H), 2.39-2.32 (m, 2H), 2.27-2.18 (m, 1H), 2.13-2.03 (m, 1H), 1.95-1.84 (m, 4H), 1.81-1.71 (m, 3H), 1.63-1.43 (m, 6H), 1.37-1.23 (m, 3H).

### Example 181: Synthesis of 1-(6-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione

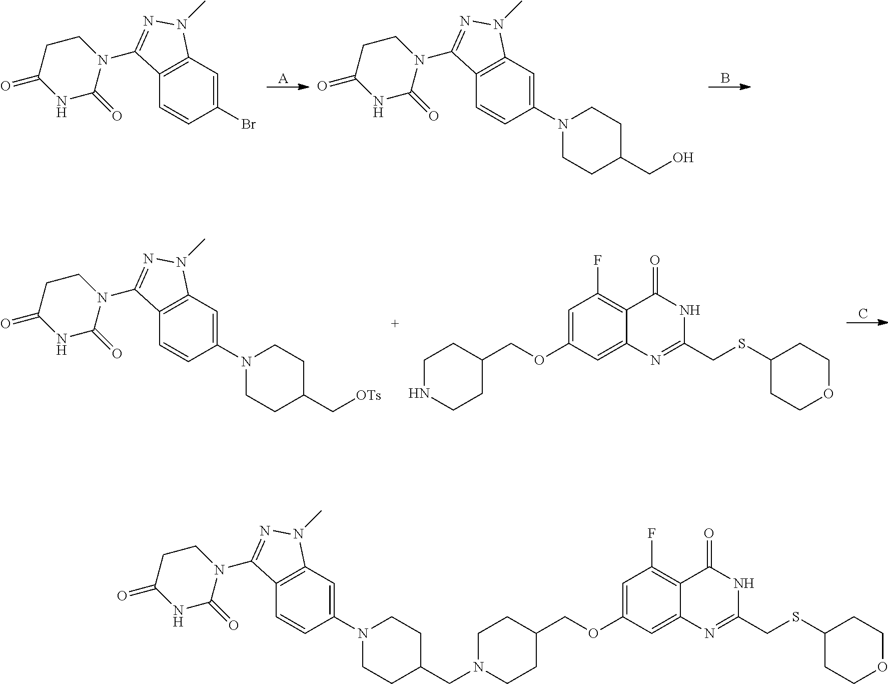

### Step A

A solution of 1-(6-bromo-1-methyl-1H-indazol-3-yl)dihydropyrimidine-2,4(1H,3H)-dione (650 mg, 2.01 mmol, 1 equiv), piperidin-4-ylmethanol (255 mg, 2.21 mmol, 1.1 equiv), Cs₂CO₃ (1.31 g, 4.02 mmol, 2 equiv) and Pd PEPPSI IPentCl (3 mg, 0.003 mmol, 0.1 equiv) in dioxane (10 mL) was stirred overnight at 85° C. under nitrogen atmosphere. The mixture was concentrated and purified by reverse-phase flash chromatography with the following conditions: column, C18 silica gel; mobile phase, MeCN in water (10 mmol/L NH₄HCO₃), 10% to 50% gradient in 10 min; detector, UV 254 nm to give 1-(6-(4-(hydroxymethyl)piperidin-1-yl)-1-methyl-1H-indazol-3-yl)dihydropyrimidine-2,4(1H,3H)-dione (300 mg, 42%) as a yellow solid. LCMS (ESI, m/z): 358.10 [M+H]⁺.

### Step B

A solution of 1-(6-(4-(hydroxymethyl)piperidin-1-yl)-1-methyl-1H-indazol-3-yl)dihydropyrimidine-2,4(1H,3H)-dione (100 mg, 0.280 mmol, 1 equiv), TsCl (64 mg, 0.34 mmol, 1.2 equiv), TEA (85 mg, 0.84 mmol, 3 equiv) and DMAP (3 mg, 0.03 mmol, 0.1 equiv) in DCM (5 mL) was stirred overnight. The mixture was concentrated and the residue was purified by silica gel column chromatography, eluting with PE/EtOAc (1:9) to afford (1-(3-(2,4-dioxotetrahydropyrimidin-1(2H)-yl)-1-methyl-1H-indazol-6-yl)piperidin-4-yl)methyl 4-methylbenzenesulfonate (70 mg, 49%) as a white solid. LCMS (ESI, m/z): 512.10 [M+H]⁺.

### Step C

A solution of (1-(3-(2,4-dioxotetrahydropyrimidin-1(2H)-yl)-1-methyl-1H-indazol-6-yl)piperidin-4-yl)methyl 4-methylbenzenesulfonate (70 mg, 0.14 mmol, 1 equiv), 5-fluoro-7-(piperidin-4-ylmethoxy)-2-(((tetrahydro-2H-pyran-4-yl)thio)methyl)quinazolin-4(3H)-one (56 mg, 0.14 mmol, 1 equiv), K₂CO₃ (38 mg, 0.27 mmol, 2 equiv) and KI (2 mg, 0.014 mmol, 0.1 equiv) in ACN (5 mL) was stirred overnight at 60° C. The mixture was concentrated and the residue purified by reverse-phase flash chromatography with the following conditions: column, C18 silica gel; mobile phase, MeCN in water (10 mmol/L NH₄HCO₃), 10% to 50% gradient in 10 min; detector, UV 254 nm to give 1-(6-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione (17 mg, 17%) as a white solid. LCMS (ESI, m/z): 747.35 [M+H]⁺. 1H NMR (400 MHz, DMSO-d₆) 612.19 (br, 1H), 10.48 (s, 1H), 7.42 (d, J=8.9 Hz, 1H), 6.95-6.83 (m, 2H), 6.62-6.52 (m, 1H), 6.31 (s, 1H), 3.98 (d, J=5.9 Hz, 2H), 3.91-3.80 (m, 7H), 3.62 (s, 2H), 3.53-3.49 (m, 3H), 3.48-3.45 (m, 3H), 3.32-3.28 (m, 3H), 3.08-3.04 (m, 1H), 2.98-2.92 (m, 2H), 2.78-2.71 (m, 2H), 2.48-2.37 (m, 2H), 2.34-2.26 (m, 1H), 2.20-2.12 (m, 1H), 2.02-1.95 (m, 1H), 1.91-1.84 (m, 2H), 1.82-1.75 (m, 2H), 1.66-1.60 (m, 2H), 1.52-1.41 (m, 2H), 1.40-1.29 (m, 2H).

Examples 182-183 were synthesized according to the procedure described for the synthesis of 1-(6-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione Example 181 using appropriate building blocks and modified reaction conditions (such as reagents, reagent ratio, temperature, and reaction time) and purification conditions as needed.

| Ex- | | |
|---|---|---|
| am- | | |
| ple | Structure | Analytical Data |
| Ex- am- ple 182 | 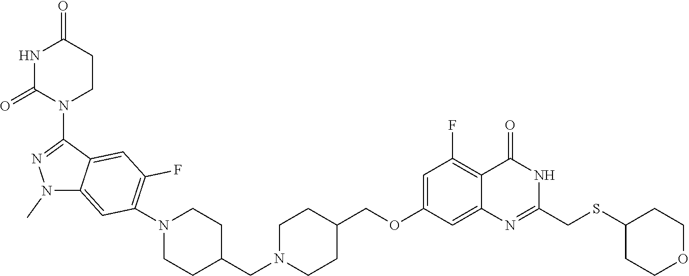 [SMILES: Cn1nc(N2CCC(=O)NC2=O)c2cc(F)c(N3CCC(CN4CCC(COc5cc(F)c6c(=O)[nH]c(CSC7CCOCC7)nc6c5)CC4)CC3)cc21] 1-(5-fluoro-6-(4-((4-(((5-fluoro-4-oxo-2- (((tetrahydro-2H-pyran-4-yl)thio)methyl)-3,4- dihydroquinazolin-7-yl)oxy)methyl)piperidin-1- yl)methyl)piperidin-1-yl)-1-methyl-1H-indazol-3- yl)dihydropyrimidine-2,4(1H,3H)-dione | LCMS (ESI, m/z): 765.25 [M + H]⁺. 1H NMR (400 MHz, DMSO-d6) δ 12.16 (s, 1H), 10.49 (s, 1H), 7.28 (d, J = 14.1 Hz, 1H), 6.88 (d, J = 9.1 Hz, 2H), 6.62 (d, J = 7.3 Hz, 1H), 3.98 (d, J = 5.8 Hz, 2H), 3.96- 3.86 (m, 5H), 3.82 (d, J = 11.4 Hz, 2H), 3.62 (s, 2H), 3.54-3.44 (m, 3H), 3.40-3.33 (m, 2H), 3.09 (t, J = 9.7 Hz, 2H), 2.93 (d, J = 10.8 Hz, 2H), 2.73 (t, J = 6.6 Hz, 2H), 2.40- 2.31 (m, 2H), 2.30-2.11 (m, 1H), 2.12-2.05 (m, 1H), 1.93-1.85 (m, 4H), 1.80-1.70 (m, 3H), 1.62- 1.50 (m, 3H), 1.52-1.42 (m, 2H), 1.32-1.20 (m, 2H). |
| Ex- am- ple 183 | 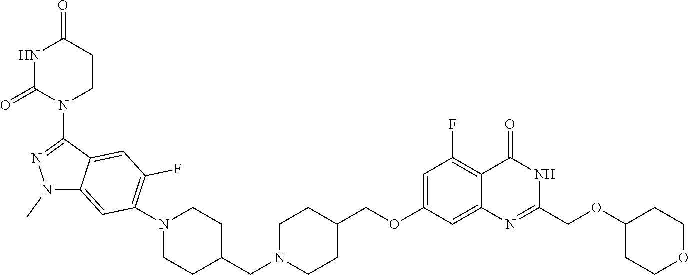 [SMILES: Cn1nc(N2CCC(=O)NC2=O)c2cc(F)c(N3CCC(CN4CCC(COc5cc(F)c6c(=O)[nH]c(COC7CCOCC7)nc6c5)CC4)CC3)cc21] 1-(5-fluoro-6-(4-((4-(((5-fluoro-4-oxo-2- (((tetrahydro-2H-pyran-4-yl)oxy)methyl)-3,4- dihydroquinazolin-7-yl)oxy)methyl)piperidin-1- yl)methyl)piperidin-1-yl)-1-methyl-1H-indazol-3- yl)dihydropyrimidine-2,4(1H,3H)-dione | LCMS (ESI, m/z): 749.30 [M + H]⁺. 1H NMR (400 MHz, DMSO-d6) δ 12.00 (br, 1H), 10.49 (s, 1H), 7.28 (d, J = 14.1 Hz, 1H), 6.96- 6.86 (m, 2H), 6.62 (d, J = 7.4 Hz, 1H), 4.38 (s, 2H), 3.98 (d, J = 5.9 Hz, 2H), 3.96-3.86 (m, 5H), 3.85-3.80 (m, 2H), 3.70- 3.61 (m, 1H), 3.56-3.4 (m, 3H), 3.39-3.31 (m, 2H), 3.14-3.05 (m, 1H), 2.92 (d, J = 10.8 Hz, 2H), 2.73 (t, J = 6.7 Hz, 2H), 2.36-2.30 (m, 2H), 2.32- 2.21 (m, 1H), 2.18-2.10 (m, 1H), 1.84-1.86 (m, 4H), 1.78-1.70 (m, 3H), 1.68-1.60 (m, 3H), 1.55- 1.42 (m, 2H), 1.34-1.20 (m, 2H). |

### Example 184: Synthesis of 1-(6-(4-(((5-fluoro-4-oxo-2-(((tetrahydro-2H-pyran-4-yl)oxy)methyl)-3,4-dihydroquinazolin-7-yl)oxy)methyl)piperidin-1-yl)-1-methyl-1H-indazol-3-yl)dihydropyrimidine-2,4(1H,3H)-dione

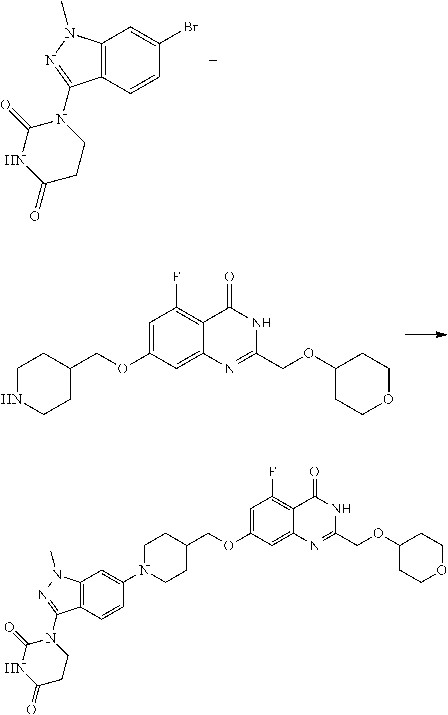 [SMILES: Cn1nc(N2CCC(=O)NC2=O)c2ccc(Br)cc21 ; O=c1[nH]c(COC2CCOCC2)nc2cc(OCC3CCNCC3)cc(F)c12 ; Cn1nc(N2CCC(=O)NC2=O)c2ccc(N3CCC(COc4cc(F)c5c(=O)[nH]c(COC6CCOCC6)nc5c4)CC3)cc21]

A solution of 1-(6-bromo-1-methyl-1H-indazol-3-yl)dihydropyrimidine-2,4(1H,3H)-dione (100 mg, 0.309 mmol, 1 equiv), 5-fluoro-7-(piperidin-4-ylmethoxy)-2-(((tetrahydro-2H-pyran-4-yl)oxy)methyl)quinazolin-4(3H)-one (121 mg, 0.309 mmol, 1 equiv), t-BuOK (70 mg, 0.62 mmol, 2 equiv) and (DiMelHeptCl)Pd(cinnamyl)Cl₂ (65 mg, 0.062 mmol, 0.2 equiv) in dioxane (4 mL) was stirred overnight at 90° C. under nitrogen atmosphere. The mixture was concentrated and the residue purified by reverse-phase flash chromatography with the following conditions: column, C18 silica gel; mobile phase, MeCN in water (10 mmol/L NH₄HCO₃), 10% to 50% gradient in 10 min; detector, UV 254 nm. The crude product was purified by prep-HPLC with the following conditions (Column: Xselect CSH F-phenyl OBD Column 19*250 mm, 5 μm; Mobile Phase A: water (0.1% FA), Mobile Phase B: ACN; Flow rate: 25 mL/min; Gradient: 15% B to 25% B in 10 ms; Wavelength: 254/220 nm; RT (min): 11.8) to give 1-(6-(4-(((5-fluoro-4-oxo-2-(((tetrahydro-2H-pyran-4-yl)oxy)methyl)-3,4-dihydroquinazolin-7-yl)oxy)methyl)piperidin-1-yl)-1-methyl-1H-indazol-3-yl)dihydropyrimidine-2,4(1H,3H)-dione (2 mg, 1%) as a white solid. LCMS (ESI, m/z): 634.30 [M+H]⁺. 1HNMR (400 MHz, DMSO-d₆) δ 12.01 (s, 1H), 10.49 (s, 1H), 7.45 (d, J=9.1 Hz, 1H), 6.99-6.90 (7, 3H), 6.80 (s, 1H), 4.38 (s, 2H), 4.06 (d, J=6.3 Hz, 2H), 3.91-3.87 (m, 4H), 3.86-3.81 (m, 2H), 3.72-3.63 (m, 2H), 3.55-3.47 (m, 3H), 2.87-2.73 (m, 4H), 2.04-1.95 (m, 1H), 1.90 (d, J=12.8 Hz, 4H), 1.55-1.43 (m, 4H), 1.40-1.32 (n, 1H).

Example 185 was synthesized according to the procedure described for the synthesis of 1-(6-(4-(((5-fluoro-4-oxo-2-(((tetrahydro-2H-pyran-4-yl)oxy)methyl)-3,4-dihydroquinazolin-7-yl)oxy)methyl)piperidin-1-yl)-1-methyl-1H-indazol-3-yl)dihydropyrimidine-2,4(1H,3H)-dione Example 184: using appropriate building blocks and modified reaction conditions (such as reagents, reagent ratio, temperature, and reaction time) and purification conditions as needed.

| Example | Structure | Analytical Data |
|---|---|---|
| Example 185 | 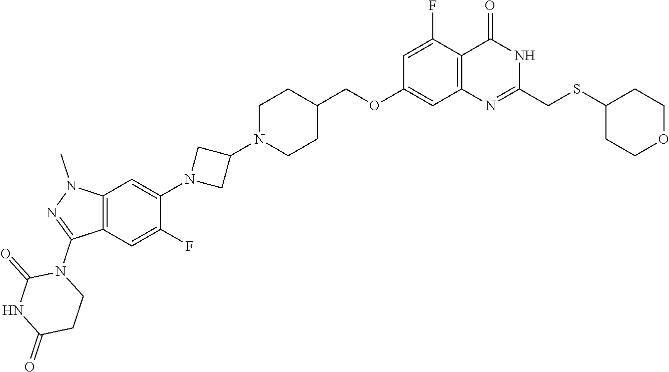 [SMILES: Cn1nc(N2CCC(=O)NC2=O)c2cc(F)c(N3CC(N4CCC(COc5cc(F)c6c(=O)[nH]c(CSC7CCOCC7)nc6c5)CC4)C3)cc21] 1-(5-fluoro-6-(3-(4-(((5-fluoro-4-oxo-2- (((tetrahydro-2H-pyran-4-yl)thio)methyl)-3,4- dihydroquinazolin-7-yl)oxy)methyl)piperidin-1- yl)azetidin-1-yl)-1-methyl-1H-indazol-3- yl)dihydropyrimidine-2,4(1H,3H)-dione | LCMS (ESI, m/z): 723.45 [M + H]⁺. 1H NMR (400 MHz, DMSO-d6) δ 12.10 (br, 1H), 10.50 (s, 1H), 7.27 (d, J = 12.7 Hz, 1H), 6.97- 6.77 (m, 2H), 6.55 (d, J = 7.4 Hz, 1H), 4.12- 4.08 (m, 2H), 3.98 (d, J = 5.8 Hz, 2H), 3.89-3.78 (m, 5H), 3.73 (t, J = 6.8 Hz, 2H), 3.61 (s, 2H), 3.33-3.28 (m, 3H), 3.28-3.21 (m, 2H), 3.10- 2.99 (m, 1H), 2.84 (d, J = 10.6 Hz, 2H), 2.73 (t, J = 6.7 Hz, 2H), 2.10-1.54 (m, 7H), 1.54-1.38 (m, 2H), 1.38-1.21 (m, 2H). |

### Example 186: Synthesis of 1-(6-(1-(2-((5-fluoro-4-oxo-2-(((tetrahydro-2H-pyran-4-yl)thio)methyl)-3,4-dihydroquinazolin-7-yl)oxy)ethyl)piperidin-4-yl)-1-methyl-1H-indazol-3-yl)dihydropyrimidine-2,4(1H,3H)-dione

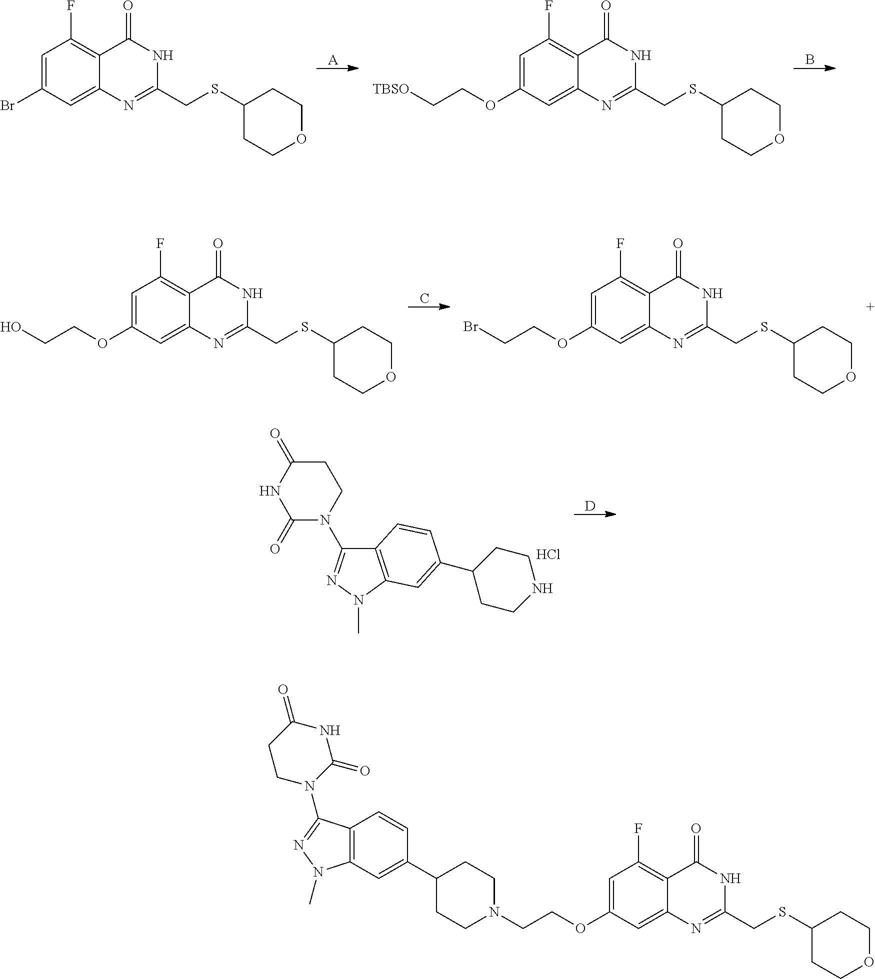

### Step A

A solution of 7-bromo-5-fluoro-2-(((tetrahydro-2H-pyran-4-yl)thio)methyl)quinazolin-4(3H)-one (1.5 g, 4.02 mmol, 1 equiv), 2-((tert-butyldimethylsilyl)oxy)ethan-1-ol (0.71 g, 4.02 mmol, 1 equiv), Pd₂(allyl)₂Cl₂ (0.15 g, 0.40 mmol, 0.1 equiv), Cs₂CO₃ (2.62 g, 8.04 mmol, 2 equiv) and RockPhos (0.02 g, 0.04 mmol, 0.01 equiv) in toluene (20 mL) was overnight at 80° C. under nitrogen atmosphere. After concentration, the crude product was purified by silica gel column eluting with DCM/MeOH (93:7) to afford 7-(2-((tert-butyldimethylsilyl)oxy)ethoxy)-5-fluoro-2-(((tetrahydro-2H-pyran-4-yl)thio)methyl)quinazolin-4(3H)-one (780 mg, 41%) as a yellow solid. LCMS (ESI, m/z):469.75 [M+H]⁺.

### Step B

A solution of 7-(2-((tert-butyldimethylsilyl)oxy)ethoxy)-5-fluoro-2-(((tetrahydro-2H-pyran-4-yl)thio)methyl)quinazolin-4(3H)-one (768 mg, 1.64 mmol, 1 equiv) in HCl in 1,4-dioxane (20 mL, 4 M) was stirred for 30 min. The mixture was concentrated to dryness to afford 5-fluoro-7-(2-hydroxyethoxy)-2-(((tetrahydro-2H-pyran-4-yl)thio)methyl)quinazolin-4(3H)-one (850 mg) as a yellow solid. The crude product was used in the next step without further purification. LCMS (ESI, m/z): 355.15 [M+H]⁺.

### Step C

A solution of 5-fluoro-7-(2-hydroxyethoxy)-2-(((tetrahydro-2H-pyran-4-yl)thio)methyl)quinazolin-4(3H)-one (840 mg, 2.37 mmol, 1 equiv) and phosphorus tribromide (3.21 g, 11.9 mmol, 5 equiv) in ACN (20 mL) was stirred overnight at 60° C. After cooling to room temperature, the reaction was quenched with water and extracted with DCM (3×100 mL) The organics were dried over anhydrous sodium sulfate, filtered and concentrated to afford 7-(2-bromoethoxy)-5-fluoro-2-(((tetrahydro-2H-pyran-4-yl)thio)methyl)quinazolin-4(3H)-one (220 mg, 22%) as a white solid. LCMS (ESI, m/z): 417.10 [M+H]⁺.

### Step D

A solution of 7-(2-bromoethoxy)-5-fluoro-2-(((tetrahydro-2H-pyran-4-yl)thio)methyl)quinazolin-4(3H)-one (100 mg, 0.27 mmol, 1 equiv), 1-(1-methyl-6-(piperidin-4-yl)-1H-indazol-3-yl)dihydropyrimidine-2,4(1H,3H)-dione (87.7 mg, 0.27 mmol, 1 equiv) and DIEA (104 mg, 0.80 mmol, 3 equiv) in DMSO (5 mL) was stirred for 2 hours at 80° C. After concentration, the residue was purified by C18 reverse phase chromatography eluting with water/ACN (78:22) to afford 1-(6-(1-(2-((5-fluoro-4-oxo-2-(((tetrahydro-2H-pyran-4-yl)thio)methyl)-3,4-dihydroquinazolin-7-yl)oxy)ethyl)piperidin-4-yl)-1-methyl-1H-indazol-3-yl)dihydropyrimidine-2,4(1H,3H)-dione (30 mg, 17%) as a yellow solid. LCMS (ESI, m/z): 664.25 [M+H]⁺. 1H NMR (300 MHz, DMSO-d₆) δ 12.11 (br, 1H), 10.52 (s, 1H), 7.60-7.48 (m, 1H), 7.45 (s, 1H), 7.12-7.00 (m, 1H), 6.98-6.87 (m, 2H), 4.33-4.23 (m, 2H), 3.97-3.71 (m, 7H), 3.63 (s, 2H), 3.40-3.26 (m, 3H), 3.15-3.01 (m, 4H), 2.84-2.66 (m, 3H), 2.71-2.60 (m, 1H), 2.27-2.13 (m, 1H), 1.95-1.69 (m, 6H), 1.55-1.36 (m, 2H).

### Example 187: Synthesis of 1-(6-(1′-(2-((5-fluoro-4-oxo-2-(((tetrahydro-2H-pyran-4-yl)thio)methyl)-3,4-dihydroquinazolin-7-yl)oxy)ethyl)-[1,4′-bipiperidin]-4-yl)-1-methyl-1H-indazol-3-yl)dihydropyrimidine-2,4(1H,3H)-dione diformate

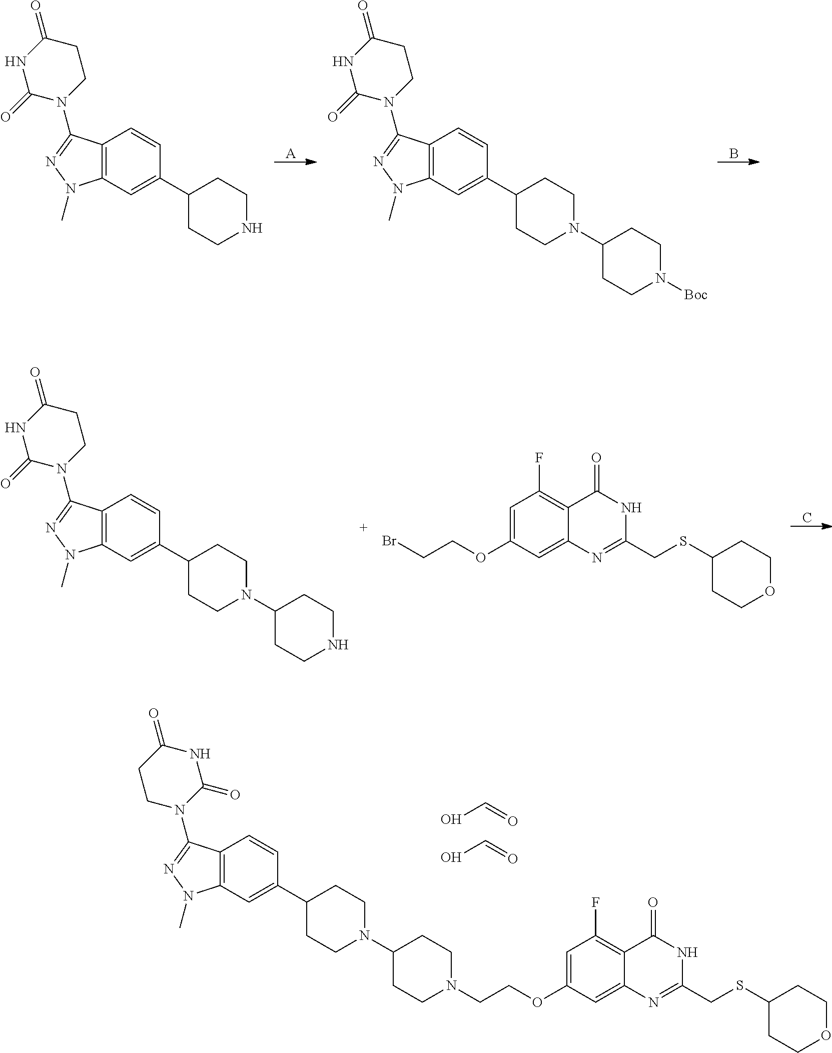

### Step A

A solution of 1-[1-methyl-6-(piperidin-4-yl)indazol-3-yl]-1,3-diazinane-2,4-dione (1.1 g, 3.36 mmol, 1 equiv) in DCE (70 mL) was treated with tert-butyl 4-oxopiperidine-1-carboxylate (4.69 g, 23.5 mmol, 7 equiv) overnight at 60° C. followed by the addition of STAB (1.42 g, 6.72 mmol, 2 equiv) portion-wise at room temperature. The mixture was stirred for 4 hours at room temperature. After concentration, the residue was purified by silica gel column chromatography, eluting with CH₂Cl₂/MeOH (10:1) to afford tert-butyl 4-(3-(2,4-dioxotetrahydropyrimidin-1(2H)-yl)-1-methyl-1H-indazol-6-yl)-[1,4′-bipiperidine]-1′-carboxylate (800 mg, 47%) as a brown oil. LCMS (ESI, m/z): 511.25 [M+H]⁺.

### Step B

A solution of tert-butyl 4-(3-2,4-dioxotetrahydropyrimidin-1(2H)-yl)-1-methyl-1H-indazol-6-yl)-[1,4′-bipiperidine]-1′-carboxylate (300 mg, 0.59 mmol, 1 equiv) in HCl in 1,4-dioxane (20 mL, 4 M) was stirred for 1 hour. The mixture was neutralized to pH 6 with saturated aqueous NaHCO₃. The crude product was purified by Prep-HPLC with the following conditions (Column: XBridge Prep Phenyl OBD Column 19*250 nm, 5 μm; Mobile Phase A: Water (10 mmol/L NH₄HCO₃), Mobile Phase B: ACN; Flow rate: 20 mL/min; Gradient: 10% B to 16% B in 10 min; Wavelength: 254 nm/220 nm; RT (min): 17.12). This afforded 1-(6-([1,4′-bipiperidin]-4-yl)-1-methyl)-1H-indazol-3-yl)dihydropyrimidine-2,4(1H,3H)-dione (50 mg, 21%) as a white solid. LCMS (ESI, m/z): 411.20 [M+H]⁺.

### Step C

A solution of 1-(6-([1,4′-bipiperidin]-4-yl)-1-methyl-1H-indazol-3-yl)dihydropyrimidine-2,4(1H,3H)-dione (50 mg, 0.122 mmol, 1 equiv), DIEA (47.2 mg, 0.366 mmol, 3 equiv) and 7-(2-bromoethoxy)-5-fluoro-2-(((tetrahydro-2H-pyran-4-yl)thio)methyl)quinazolin-4(3H)-one (50.8 mg, 0.122 mmol, 1 equiv) in DMSO (3 mL) was stirred for 2 hours at 80° C. The residue was purified by reverse flash chromatography with the following conditions: (column, C18 silica gel; mobile phase, MeCN in Water (0.1% FA), 0% to 20% gradient in 15 min; detector, UV 254 nm). This afforded 1-(6-(1′-(2-((5-fluoro-4-oxo-2-(((tetrahydro-2H-pyran-4-yl)thio)methyl)-3,4-dihydroquinazolin-7-yl)oxy)ethyl)-[1,4′-bipiperidin]-4-yl)-1-methyl-1H-indazol-3-yl)dihydropyrimidine-2,4(1H,3H)-dione diformate (27 mg, 28%) as a brown solid. LCMS (ESI, m/z): 747.40 [M+H]⁺. 1H NMR (400 MHz, DMSO-d₆) δ 10.54 (s, 1H), 8.21 (s, 2H) HCOOH, 7.64-7.52 (m, 1H), 7.44 (s, 1H), 7.05 (d, J=4.0 Hz, 1H), 7.00-6.75 (m, 2H), 4.29-4.20 (m, 2H), 3.96 (s, 3H), 3.94-3.90 (m, 2H), 3.85-3.80 (m, 2H), 3.36-3.30 (m, 2H), 3.12-3.00 (m, 5H), 2.80-2.65 (m, 5H), 2.56-2.53 (m, 1H), 2.48-2.38 (m, 3H), 2.13-1.99 (m, 2H), 1.98-1.70 (m, 8H), 1.60-1.39 (m, 4H).

Examples 188-189 were synthesized according to the procedure described for the synthesis of N-(6-((2-(2,6-dioxopiperidin-3-yl)-1,3-dioxoisoindolin-4-yl)amino)hexyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide (Example 1) using appropriate building blocks and modified reaction conditions (such as reagents, reagent ratio, temperature, and reaction time) and purification conditions as needed.

| Example | Structure | Analytical Data |
|---|---|---|
| Example 188 | 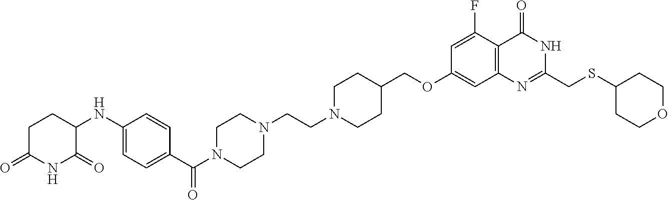 [SMILES: O=C1CCC(Nc2ccc(C(=O)N3CCN(CCN4CCC(COc5cc(F)c6c(=O)[nH]c(CSC7CCOCC7)nc6c5)CC4)CC3)cc2)C(=O)N1] 3-((4-(4-(2-(4-(((5-fluoro-4-oxo-2-(((tetrahydro-2H-pyran- 4-yl)thio)methyl)-3,4-dihydroquinazolin-7- yl)oxy)methyl)piperidin-1-yl)ethyl)piperazine-1- carbonyl)phenyl)amino)piperidine-2,6-dione | LCMS (ESI, m/z): 750.40 [M + H]⁺. 1H NMR (400 MHz, DMSO-d6) δ 12.15 (s, 1H), 10.81 (s, 1H), 7.21-7.14 (m, 2H), 6.91- 6.82 (m, 2H), 6.71- 6.65 (m, 2H), 6.29 (d, J = 7.8 Hz, 1H), 4.44-4.38 (m, 1H), 3.97 (d, J = 5.9 Hz, 2H), 3.84- 3.79 (m, 2H), 3.61 (s, 2H), 3.48 (s, 4H), 3.37-3.34 (m, 1H), 3.31- 3.29 (m, 1H), 3.12- 3.00 (m, 1H), 2.90 (d, J = 10.9 Hz, 2H), 2.83- 2.71 (m, 1H), 2.63- 2.59 (m, 1H), 2.58-2.53 (m, 1H), 2.45-2.36 (m, 7H), 2.15- 2.07 (m, 1H), 1.98- 1.86 (m, 5H), 1.78-1.72 (m, 3H), 1.52-1.39 (m, 2H), 1.35-1.23 (m, 2H). |
| Example 189 | 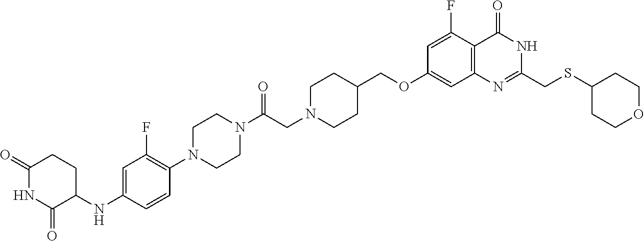 [SMILES: O=C1CCC(Nc2ccc(N3CCN(C(=O)CN4CCC(COc5cc(F)c6c(=O)[nH]c(CSC7CCOCC7)nc6c5)CC4)CC3)c(F)c2)C(=O)N1] 3-((3-fluoro-4-(4-(2-(4-(((5-fluoro-4-oxo-2-(((tetrahydro- 2H-pyran-4-yl)thio)methyl)-3,4-dihydroquinazolin-7- yl)oxy)methyl)piperidin-1-yl)acetyl)piperazin-1- yl)phenyl)amino)piperidine-2,6-dione | LCMS (ESI, m/z): 754.35 [M + H]⁺. 1H NMR (300 MHz, DMSO-d6) δ 12.14 (s, 1H), 10.76 (s, 1H), 6.93-6.79 (m, 3H), 6.53 (d, J = 15.5 Hz, 1H), 6.44 (d, J = 9.4 Hz, 1H), 5.85 (d, J = 7.6 Hz, 1H), 4.41- 4.20 (m, 1H), 4.13 (d, J = 5.8 Hz, 2H), 3.92-3.78 (m, 2H), 3.70-3.55 (m, 6H), 3.37-3.22 (m, 2H), 3.18- 3.12 (m, 1H), 3.11- 3.00 (m, 1H), 2.97-2.65 (m, 7H), 2.64-2.60 (m, 1H), 2.14-1.94 (m, 3H), 1.92-1.75 (m, 6H), 1.55-1.28 (m, 4H). |

Examples 190-191 were synthesized according to the procedure described for the synthesis of 2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione (Example 23) using appropriate building blocks and modified reaction conditions (such as reagents, reagent ratio, temperature, and reaction time) and purification conditions as needed.

| Example | Structure | Analytical Data |
|---|---|---|
| Example 190 | 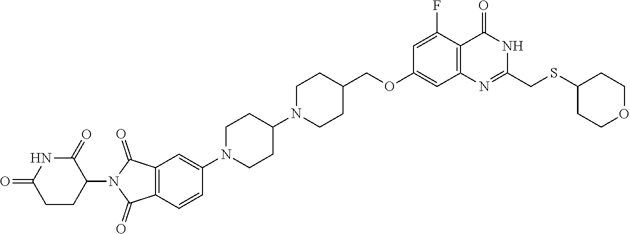 [SMILES: O=C1CCC(N2C(=O)c3ccc(N4CCC(N5CCC(COc6cc(F)c7c(=O)[nH]c(CSC8CCOCC8)nc7c6)CC5)CC4)cc3C2=O)C(=O)N1] 2-(2,6-dioxopiperidin-3-yl)-5-(4-(((5-fluoro-4-oxo-2- (((tetrahydro-2H-pyran-4-yl)thio)methyl)-3,4- dihydroquinazolin-7-yl)oxy)methyl)-[1,4′-bipiperidin]-1′- yl)isoindoline-1,3-dione | LCMS (ESI, m/z): 747.25 [M + H]⁺. 1H NMR (400 MHz, DMSO-d6) δ 12.16 (s, 1H), 11.08 (s, 1H), 7.66 (d, J = 8.5 Hz, 1H), 7.32 (s, 1H), 7.28-7.20 (m, 1H), 6.88 (t, J = 5.8 Hz, 2H), 5.07 (dd, J = 12.8, 5.4 Hz, 1H), 4.09 (d, J = 12.8 Hz, 2H), 3.97 (d, J = 5.7 Hz, 2H), 3.95-3.80 (m, 2H), 3.62 (s, 2H), 3.19-2.81 (m, 8H), 2.70-2.55 (m, 3H), 2.17 (t, J = 11.2 Hz, 2H), 2.05-1.95 (m, 1H), 1.89-1.69 (m, 7H), 1.56-1.40 (m, 4H), 1.35-1.25 (m, 2H). |
| Example 191 | 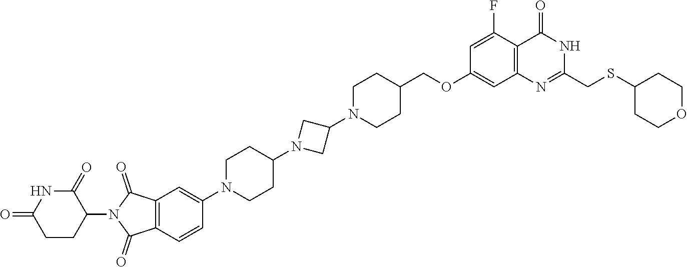 [SMILES: O=C1CCC(N2C(=O)c3ccc(N4CCC(N5CC(N6CCC(COc7cc(F)c8c(=O)[nH]c(CSC9CCOCC9)nc8c7)CC6)C5)CC4)cc3C2=O)C(=O)N1] 2-(2,6-dioxopiperidin-3-yl)-5-(4-(3-(4-(((5-fluoro-4-oxo- 2-(((tetrahydro-2H-pyran-4-yl)thio)methyl)-3,4- dihydroquinazolin-7-yl)oxy)methyl)piperidin-1- yl)azetidin-1-yl)piperidin-1-yl)isoindoline-1,3-dione | LCMS (ESI, m/z): 802.30 [M + H]⁺. 1H NMR (300 MHz, DMSO-d6) δ 12.20 (s, 1H), 11.09 (s, 1H), 7.70 (d, J = 8.5 Hz, 1H), 7.40 (s, 1H), 7.31 (d, J = 8.4 Hz, 1H), 6.92-6.85 (m, 2H), 5.08 (dd, J = 12.8, 5.4 Hz, 1H), 4.80-4.60 (m, 1H), 4.50-3.90 (m, 8H), 3.82-3.73 (m, 3H), 3.70 (s, 3H), 3.30- 3.25 (m, 3H), 3.18- 2.75 (m, 7H), 2.70- 2.50 (m, 2H), 2.15- 1.72 (m, 8H), 1.60- 1.30 (m, 5H) |

Examples 192-212 were synthesized according to the procedure described for the synthesis 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1l-yl)ethyl)piperazin-1-yl)phenyl)amino)piperidine-2,6-dione (Example 48) using appropriate building blocks and modified reaction conditions (such as reagents, reagent ratio, temperature, and reaction time) and purification conditions as needed.

| Example | Structure | Analytical Data |
|---|---|---|
| Example 192 | 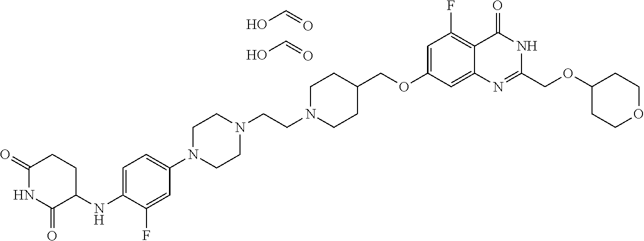 3-((2-fluoro-4-(4-(2-(4-(((5-fluoro-4-oxo-2-(((tetrahydro- 2H-pyran-4-yl)oxy)methyl)-3,4-dihydroquinazolin-7- yl)oxy)methyl)piperidin-1-yl)ethyl)piperazin-1- yl)phenyl)amino)piperidine-2,6-dione diformate | LCMS (ESI, m/z): 724.30 [M + H]⁺. 1H NMR (400 MHz, DMSO-d6) δ 12.03 (s, 1H), 10.80 (s, 1H), 8.20 (s, 2H, HCOOH), 7.01- 6.86 (m, 2H), 6.83- 6.70 (m, 2H), 6.59 (dd, J = 8.8, 2.6 Hz, 1H), 5.06 (d, J = 7.4 Hz, 1H), 4.38 (s, 2H), 4.31-4.23 (m, 1H), 3.99 (d, J = 6.0 Hz, 2H), 3.86-3.79 (m, 4H), 3.70-3.65 (m, 2H), 3.38-3.30 (m, 2H), 3.09-2.85 (m, 6H), 2.82-2.69 (m, 1H), 2.63-2.53 (m, 6H), 2.18-2.04 (m, 3H), 2.04-1.86 (m, 3H), 1.84-1.70 (m, 3H), 1.55-1.42 (m, 2H), 1.41-1.27 (m, 2H). |
| Example 193 | 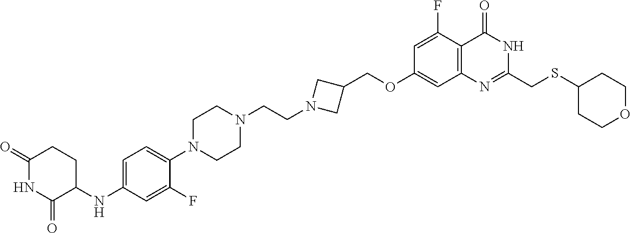 [SMILES: O=C1CCC(Nc2ccc(N3CCN(CCN4CC(COc5cc(F)c6c(=O)[nH]c(CSC7CCOCC7)nc6c5)C4)CC3)c(F)c2)C(=O)N1] 3-((3-fluoro-4-(4-(2-(3-(((5-fluoro-4-oxo-2-(((tetrahydro- 2H-pyran-4-yl)thio)methyl)-3,4-dihydroquinazolin-7- yl)oxy)methyl)azetidin-1-yl)ethyl)piperazin-1- yl)phenyl)amino)piperidine-2,6-dione | LCMS (ESI, m/z): 712.25 [M + H]⁺. 1H NMR (400 MHz, DMSO-d6) δ 6.92- 6.85 (m, 2H), 6.81 (t, J = 16 Hz, 1H), 6.51 (dd, J = 15.0, 2.6 Hz, 1H), 6.42 (dd, J = 8.7, 2.6 Hz, 1H), 5.79 (d, J = 8 Hz, 1H), 4.30-4.20 (m, 3H), 3.86-3.77 (m, 2H), 3.62 (s, 2H), 3.38-3.27 (m, 1H), 3.26-3.24 (m, 5H), 3.12-3.02 (m, 1H), 2.98 (t, J = 12 Hz, 2H), 2.87-2.76 (m, 5H), 2.75-2.66 (m, 1H), 2.62-2.52 (m, 2H), 2.32-2.25 (m, 2H), 2.28 (t, J = 10 Hz, 1H), 2.14-2.03 (m, 2H), 1.93-1.78 (m, 2H), 1.52-1.38 (m, 3H). |
| Example 194 | 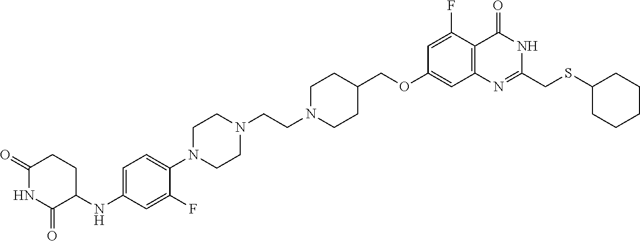 [SMILES: O=C1CCC(Nc2ccc(N3CCN(CCN4CCC(COc5cc(F)c6c(=O)[nH]c(CSC7CCCCC7)nc6c5)CC4)CC3)c(F)c2)C(=O)N1] 3-({4-[4-(2-{4-[({2-[(cyclohexylsulfanyl)methyl]-5- fluoro-4-oxo-3H-quinazolin-7-yl}oxy)methyl]piperidin-1- yl}ethyl)piperazin-1-yl]-3- fluorophenyl}amino)piperidine-2,6-dione | LCMS (ESI, m/z): 738.40 [M + H]⁺. 1H NMR (300 MHz, DMSO-d6) δ 12.14 (s, 1H), 10.77 (s, 1H), 6.94-6.76 (m, 3H), 6.53-6.37 (m, 2H), 5.80 (d, J = 7.7 Hz, 1H), 4.33-4.19 (m, 1H), 3.98 (d, J = 5.9 Hz, 2H), 3.58 (s, 2H), 2.95-2.74 (m, 7H), 2.73-2.69 (m, 2H), 2.60-2.50 (m, 6H), 2.08 (s, 2H), 1.98-1.80 (m, 5H), 1.79-1.64 (m, 5H), 1.60-1.51 (m, 1H), 1.41-1.15 (m, 8H). |
| Example 195 | 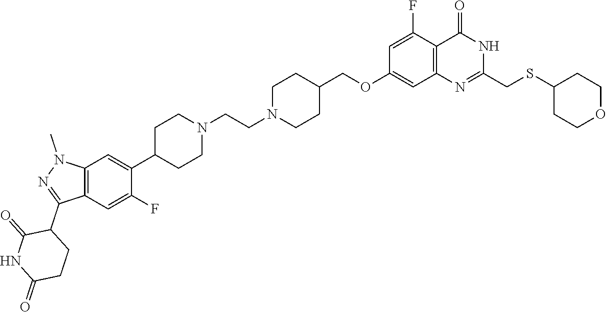 [SMILES: Cn1nc(C2CCC(=O)NC2=O)c2cc(F)c(C3CCN(CCN4CCC(COc5cc(F)c6c(=O)[nH]c(CSC7CCOCC7)nc6c5)CC4)CC3)cc21] 3-(5-fluoro-6-(1-(2-(4-(((5-fluoro-4-oxo-2-(((tetrahydro- 2H-pyran-4-yl)thio)methyl)-3,4-dihydroquinazolin-7- yl)oxy)methyl)piperidin-1-yl)ethyl)piperidin-4-yl)-1- methyl-1H-indazol-3-yl)piperidine-2,6-dione | LCMS (ESI, m/z): 778.40 [M + H]⁺. 1H NMR (400 MHz, DMSO-d6) δ 12.17 (br, 1H), 10.88 (s, 1H), 7.57 (d, J = 6.0 Hz, 1H), 7.47 (d, J = 10.8 Hz, 1H), 6.93- 6.82 (m, 2H), 4.32 (dd, J = 10.0, 5.0 Hz, 1H), 4.02-3.98 (m, 5H), 3.90-3.75 (m, 2H), 3.62 (s, 2H), 3.34 (dd, J = 11.2, 2.3 Hz, 3H), 3.13- 3.04 (m, 3H), 2.95 (d, J = 10.8 Hz, 2H), 2.90-2.85 (m, 1H), 2.74-2.54 (m, 3H), 2.48-2.32 (m, 3H), 2.20-2.07 (m, 3H), 1.99 (t, J = 11.3 Hz, 2H), 1.89-1.69 (m, 9H), 1.51-1.38 (m, 2H), 1.32-1.19 (m, 2H). |
| Example 196 | 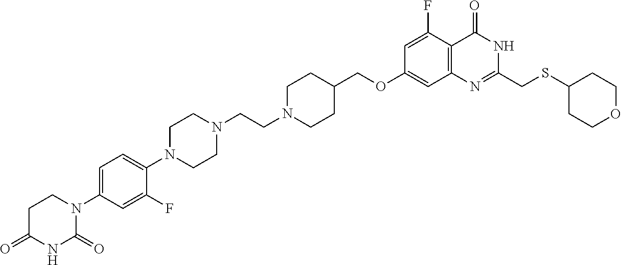 [SMILES: O=C1CCN(c2ccc(N3CCN(CCN4CCC(COc5cc(F)c6c(=O)[nH]c(CSC7CCOCC7)nc6c5)CC4)CC3)c(F)c2)C(=O)N1] 1-(3-fluoro-4-(4-(2-(4-(((5-fluoro-4-oxo-2-(((tetrahydro- 2H-pyran-4-yl)thio)methyl)-3,4-dihydroquinazolin-7- yl)oxy)methyl)piperidin-1-yl)ethyl)piperazin-1- yl)phenyl)dihydropyrimidine-2,4(1H,3H)-dione | LCMS (ESI, m/z): 726.30 [M + H]⁺. 1H NMR (300 MHz, DMSO-d6) δ 12.13 (br, 1H), 10.34 (s, 1H), 7.18 (dd, J = 14.1, 2.2 Hz, 1H), 7.12-6.96 (m, 2H), 6.93-6.82 (m, 2H), 3.98 (d, J = 5.9 Hz, 2H), 3.89-3.80 (m, 2H), 3.79-3.71 (m, 2H), 3.62 (s, 2H), 3.40-3.35 (m, 2H), 3.15-2.89 (m, 7H), 2.69 (t, J = 6.7 Hz, 2H), 2.57-2.51 (m, 4H), 2.47-2.41 (m, 4H), 2.03-1.84 (m, 4H), 1.79-1.71 (m, 3H), 1.55-1.35 (m, 4H). |
| Example 197 | 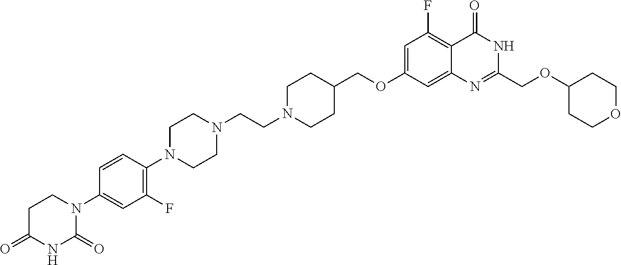 [SMILES: O=C1CCN(c2ccc(N3CCN(CCN4CCC(COc5cc(F)c6c(=O)[nH]c(COC7CCOCC7)nc6c5)CC4)CC3)c(F)c2)C(=O)N1] 1-(3-fluoro-4-(4-(2-(4-(((5-fluoro-4-oxo-2-(((tetrahydro- 2H-pyran-4-yl)oxy)methyl)-3,4-dihydroquinazolin-7- yl)oxy)methyl)piperidin-1-yl)ethyl)piperazin-1- yl)phenyl)dihydropyrimidine-2,4(1H,3H)-dione | LCMS (ESI, m/z): 710.35 [M + H]⁺. 1H NMR (300 MHz, DMSO-d6) δ 7.18 (d, J = 15, 1H), 7.10- 6.98 (m, 2H), 6.95- 6.84 (m, 2H), 4.38 (s, 2H), 3.98 (d, J = 5.8 Hz, 2H), 3.90-3.58 (m, 5H), 3.40-3.30 (m, 3H), 3.11-2.82 (m, 6H), 2.69 (t, J = 6.7 Hz, 2H), 2.57 (t, J = 4.6 Hz, 4H), 2.50-2.41 (m, 3H), 2.05-1.83 (m, 4H), 1.80-1.70 (m, 3H), 1.55-1.40 (m, 2H), 1.39-1.25 (m, 2H). |
| Example 198 | 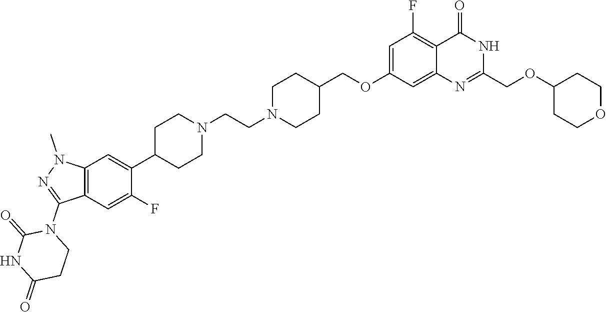 [SMILES: Cn1nc(N2CCC(=O)NC2=O)c2cc(F)c(C3CCN(CCN4CCC(COc5cc(F)c6c(=O)[nH]c(COC7CCOCC7)nc6c5)CC4)CC3)cc21] 1-(5-fluoro-6-(1-(2-(4-(((5-fluoro-4-oxo-2-(((tetrahydro- 2H-pyran-4-yl)oxy)methyl)-3,4-dihydroquinazolin-7- yl)oxy)methyl)piperidin-1-yl)ethyl)piperidin-4-yl)-1- methyl-1H-indazol-3-yl)dihydropyrimidine-2,4(1H,3H)- dione | LCMS (ESI, m/z): 763.40 [M + H]⁺. 1H NMR (400 MHz, DMSO-d6) δ 12.02 (br, 1H), 10.55 (s, 1H), 7.59 (d, J = 5.9 Hz, 1H), 7.37 (d, J = 10.8 Hz, 1H), 6.96- 6.86 (m, 2H), 4.38 (s, 2H), 4.04-3.95 (m, 5H), 3.94-3.88 (m, 2H), 3.87-3.79 (m, 2H), 3.70-3.60 (m, 1H), 3.39-3.36 (m, 2H), 3.10-3.00 (m, 2H), 3.00-2.90 (m, 2H), 2.90-2.80 (m, 1H), 2.80-2.73 (m, 2H), 2.50-2.45 (m, 3H), 2.18-2.04 (m, 2H), 2.03-1.95 (m, 2H), 1.95-1.86 (m, 3H), 1.86-1.70 (m, 7H), 1.53-1.42 (m, 2H), 1.40-1.25 (m, 2H). |
| Example 199 | 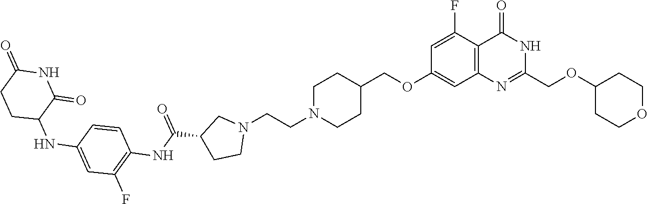 [SMILES: O=C1CCC(Nc2ccc(NC(=O)[C@H]3CCN(CCN4CCC(COc5cc(F)c6c(=O)[nH]c(COC7CCOCC7)nc6c5)CC4)C3)c(F)c2)C(=O)N1] (3S)-N-(4-((2,6-dioxopiperidin-3-yl)amino)-2- fluorophenyl)-1-(2-(4-(((5-fluoro-4-oxo-2-(((tetrahydro- 2H-pyran-4-yl)oxy)methyl)-3,4-dihydroquinazolin-7- yl)oxy)methyl)piperidin-1-yl)ethyl)pyrrolidine-3- carboxamide | LCMS (ESI, m/z): 752.40 [M + H]⁺. 1H NMR (400 MHz, DMSO-d6) δ 12.00 (s, 1H), 10.78 (s, 1H), 9.27 (s, 1H), 7.26 (t, J = 16.0 Hz, 1H), 6.95-6.85 (m, 2H), 6.54 (dd, J = 16.0, 4.0 Hz, 1H), 6.45 (dd, J = 8.0, 4.0 Hz, 1H), 6.06 (d, J = 8.0 Hz, 1H), 4.40- 4.27 (m, 3H), 3.96 (d, J = 8.0 Hz, 2H), 3.87-3.78 (m, 2H), 3.72-3.61 (m, 1H), 3.39-3.32 (m, 1H), 3.04-2.86 (m, 4H), 2.85-2.66 (m, 1H), 2.65-2.50 (m, 4H), 2.48-2.37 (m, 3H), 2.14-2.05 (m, 1H), 2.02-1.81 (m, 8H), 1.72 (d, J = 12.0 Hz, 3H), 1.55-1.41 (m, 2H), 1.35-1.22 (m, 3H). |
| Example 200 | 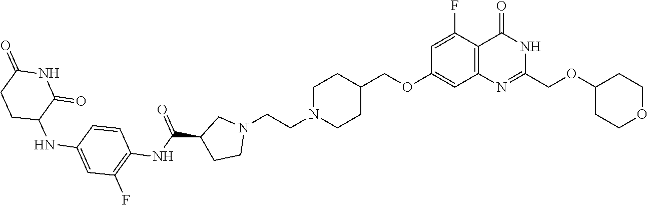 [SMILES: O=C1CCC(Nc2ccc(NC(=O)[C@@H]3CCN(CCN4CCC(COc5cc(F)c6c(=O)[nH]c(COC7CCOCC7)nc6c5)CC4)C3)c(F)c2)C(=O)N1] (3R)-N-(4-((2,6-dioxopiperidin-3-yl)amino)-2- fluorophenyl)-1-(2-(4-(((5-fluoro-4-oxo-2-(((tetrahydro- 2H-pyran-4-yl)oxy)methyl)-3,4-dihydroquinazolin-7- yl)oxy)methyl)piperidin-1-yl)ethyl)pyrrolidine-3- carboxamide. | LCMS (ESI, m/z): 752.40 [M + H]⁺. 1H NMR (400 MHz, DMSO-d6) δ 11.68 (br, 1H), 10.80 (br, 1H), 9.28 (s, 1H), 7.29-7.29 (m, 1H), 6.95-6.85 (m, 2H), 6.54 (dd, J = 13.5, 2.5 Hz, 1H), 6.45 (dd, J = 8.7, 2.5 Hz, 1H), 6.07 (d, J = 7.8 Hz, 1H), 4.38-4.31 (m, 3H), 3.96 (d, J = 5.9 Hz, 2H), 3.84- 3.78 (m, 2H), 3.66- 3.61 (m, 1H), 3.39- 3.29 (m, 2H), 3.03- 2.97 (m, 1H), 2.90 (d, J = 10.8 Hz, 2H), 2.86-2.71 (m, 2H), 2.71-2.51 (m, 4H), 2.43-2.37 (m, 3H), 2.13-2.07 (m, 1H), 2.00-1.80 (m, 8H), 1.76-1.68 (m, 3H), 1.48-1.42 (m, 2H), 1.29-1.22 (m, 2H). |
| Example 201 | 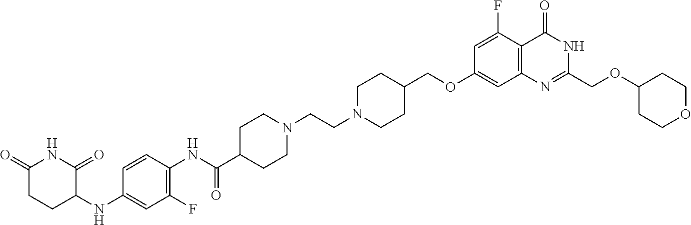 [SMILES: O=C1CCC(Nc2ccc(NC(=O)C3CCN(CCN4CCC(COc5cc(F)c6c(=O)[nH]c(COC7CCOCC7)nc6c5)CC4)CC3)c(F)c2)C(=O)N1] N-(4-((2,6-dioxopiperidin-3-yl)amino)-2-fluorophenyl)-1- (2-(4-(((5-fluoro-4-oxo-2-(((tetrahydro-2H-pyran-4- yl)oxy)methyl)-3,4-dihydroquinazolin-7- yl)oxy)methyl)piperidin-1-yl)ethyl)piperidine-4- carboxamide | LCMS (ESI, m/z): 766.40 [M + H]⁺. 1H NMR (400 MHz, DMSO-d6) δ 12.02 (br, 1H), 10.79 (s, 1H), 9.15 (s, 1H), 7.22 (t, J = 8.9 Hz, 1H), 6.92 (s, 1H), 6.88 (s, 1H), 6.53 (d, J = 12.0 Hz, 1H), 6.44 (d, J = 9.5 Hz, 1H), 6.07 (d, J = 7.9 Hz, 1H), 4.38 (s, 2H), 3.98 (d, J = 5.9 Hz, 2H), 3.86-3.80 (m, 2H), 3.71-3.62 (m, 1H), 2.95-2.89 (m, 4H), 2.80-2.66 (m, 1H), 2.41-2.29 (m, 5H), 2.12-2.05 (m, 1H), 1.99-1.84 (m, 9H), 1.81- 1.72 (m, 3H), 1.70- 1.58 (m, 2H), 1.53- 1.43 (m, 3H), 1.38- 1.22 (m, 5H). |
| Example 202 | 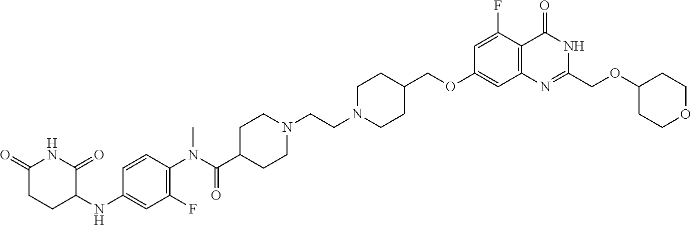 [SMILES: CN(C(=O)C1CCN(CCN2CCC(COc3cc(F)c4c(=O)[nH]c(COC5CCOCC5)nc4c3)CC2)CC1)c1ccc(NC2CCC(=O)NC2=O)cc1F] N-(4-((2,6-dioxopiperidin-3-yl)amino)-2-fluorophenyl)-1- (2-(4-(((5-fluoro-4-oxo-2-(((tetrahydro-2H-pyran-4- yl)oxy)methyl)-3,4-dihydroquinazolin-7- yl)oxy)methyl)piperidin-1-yl)ethyl)-N-methylpiperidine- 4-carboxamide | LCMS (ESI, m/z): 780.35 [M + H]⁺. 1H NMR (300 MHz, DMSO-d6) δ 11.98 (br, 1H), 10.82 (s, 1H), 7.10 (t, J = 8.9 Hz, 1H), 6.95-6.83 (m, 2H), 6.62 (d, J = 13.2 Hz, 1H), 6.55 (d, J = 8.6 Hz, 1H), 6.41 (d, J = 7.7 Hz, 1H), 4.37 (s, 3H), 3.96 (d, J = 5.9 Hz, 2H), 3.84-3.74 (m, 2H), 3.72-3.62 (m, 1H), 3.03 (s, 2H), 2.93-2.71 (m, 5H), 3.70-2.52 (m, 3H), 2.32-2.25 (m, 4H), 2.14-2.06 (m, 2H), 1.91-1.80 (m, 5H), 1.79-1.30 ((m, 12H), 1.31-1.21 (m 3H). |
| Example 203 | 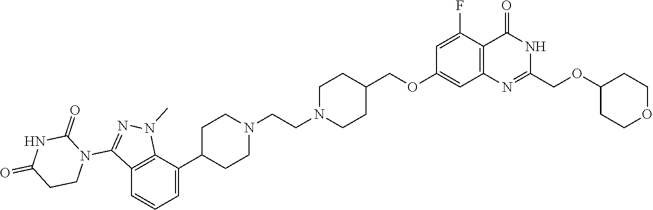 [SMILES: Cn1nc(N2CCC(=O)NC2=O)c2cccc(C3CCN(CCN4CCC(COc5cc(F)c6c(=O)[nH]c(COC7CCOCC7)nc6c5)CC4)CC3)c21] 1-(7-(1-(2-(4-(((5-fluoro-4-oxo-2-(((tetrahydro-2H-pyran- 4-yl)oxy)methyl)-3,4-dihydroquinazolin-7- yl)oxy)methyl)piperidin-1-yl)ethyl)piperidin-4-yl)-1- methyl-1H-indazol-3-yl)dihydropyrimidine-2,4(1H,3H)- dione | LCMS (ESI, m/z): 745.40 [M + H]⁺. 1H NMR (300 MHz, DMSO-d6) δ 12.01 (br, 1H), 10.55 (s, 1H), 7.55-7.45 (d, J = 6.4 Hz, 1H 1H), 7.29 (d, J = 7.2 Hz, 1H), 7.18-7.03 (m, 1H), 7.01-6.81 (m, 2H), 4.42 (s, 2H), 4.21 (s, 3H), 3.99 (d, J = 6.4 Hz, 2H), 3.91- 3.80 (m, 4H), 3.79- 3.65 (m, 1H), 3.41- 3.21 (m, 2H), 3.06 (d, J = 11.2 Hz, 2H), 2.94 (d, J = 10.7 Hz, 2H), 2.77 (t, J = 6.4 Hz, 2H), 2.49- 2.41 (m, 3H), 2.27 (t, J = 10.8 Hz, 2H), 2.01- 1.69 (m, 10H), 1.62- 1.41 (m, 2H), 1.42- 1.20 (m, 3H). |
| Example 204 | 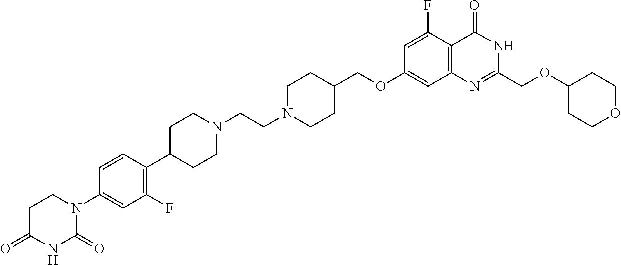 [SMILES: O=C1CCN(c2ccc(C3CCN(CCN4CCC(COc5cc(F)c6c(=O)[nH]c(COC7CCOCC7)nc6c5)CC4)CC3)c(F)c2)C(=O)N1] 1-(3-fluoro-4-(1-(2-(4-(((5-fluoro-4-oxo-2-(((tetrahydro- 2H-pyran-4-yl)oxy)methyl)-3,4-dihydroquinazolin-7- yl)oxy)methyl)piperidin-1-yl)ethyl)piperidin-4- yl)phenyl)dihydropyrimidine-2,4(1H,3H)-dione. | LCMS (ESI, m/z): 709.40 [M + H]⁺. 1H NMR (300 MHz, DMSO-d6) δ 11.99 (s, 1H), 10.39 (s, 1H), 7.34 (t, J = 8.5 Hz, 1H), 7.27-7.08 (m, 2H), 6.96-6.84 (m, 2H), 4.38 (s, 2H), 3.98 (d, J = 5.8 Hz, 2H), 3.89-3.74 (m, 4H), 3.74-3.61 (m, 1H), 3.41-3.30 (m, 2H), 2.99 (d, J = 11.0 Hz, 2H), 2.91 (d, J = 10.7 Hz, 2H), 2.89-2.71 (m, 3H), 2.44 (s, 4H), 2.12- 1.84 (m, 6H), 1.79- 1.64 (m, 7H), 1.57- 1.39 (m, 2H), 1.30- 1.21 (m, 2H). |
| Example 205 | 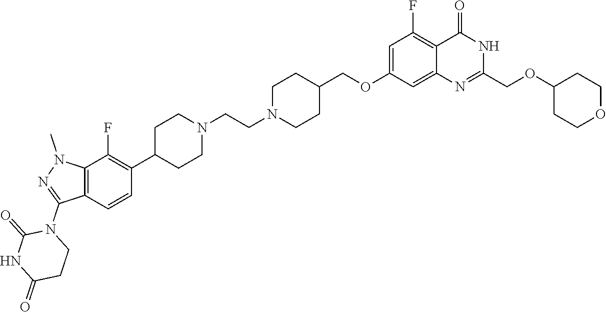 [SMILES: Cn1nc(N2CCC(=O)NC2=O)c2ccc(C3CCN(CCN4CCC(COc5cc(F)c6c(=O)[nH]c(COC7CCOCC7)nc6c5)CC4)CC3)c(F)c21] 1-(7-fluoro-6-(1-(2-(4-(((5-fluoro-4-oxo-2-(((tetrahydro- 2H-pyran-4-yl)oxy)methyl)-3,4-dihydroquinazolin-7- yl)oxy)methyl)piperidin-1-yl)ethyl)piperidin-4-yl)-1- methyl-1H-indazol-3-yl)dihydropyrimidine-2,4(1H,3H)- dione | LCMS (ESI, m/z): 763.40 [M + H]⁺. 1H NMR (400 MHz, DMSO-d6) δ 12.03 (br, 1H), 10.60 (s, 1H), 7.40 (d, J = 8.5 Hz, 1H), 7.07 (dd, J = 8.6, 5.7 Hz, 1H), 6.98-6.88 (m, 2H), 4.38 (s, 2H), 4.11 (s, 3H), 3.99 (d, J = 5.9 Hz, 2H), 3.91 (t, J = 6.7 Hz, 2H), 3.85- 3.78 (m, 2H), 3.72- 3.65 (m, 1H), 3.29- 3.20 (m, 6H), 3.08- 2.90 (m, 5H), 2.76 (t, J = 6.7 Hz, 2H), 2.13 (t, J = 11.2 Hz, 2H), 2.03 (t, J = 11.5 Hz, 2H), 1.90 (dd, J = 12.9, 3.7 Hz, 2H), 1.86-1.67 (m, 7H), 1.54-1.41 (m, 2H), 1.36-1.25 (m, 2H). |
| Example 206 | 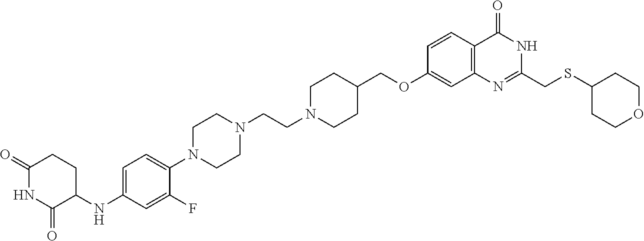 [SMILES: O=C1CCC(Nc2ccc(N3CCN(CCN4CCC(COc5ccc6c(=O)[nH]c(CSC7CCOCC7)nc6c5)CC4)CC3)c(F)c2)C(=O)N1] 3-((3-fluoro-4-(4-(2-(4-(((4-oxo-2-(((tetrahydro-2H- pyran-4-yl)thio)methyl)-3,4-dihydroquinazolin-7- yl)oxy)methyl)piperidin-1-yl)ethyl)piperazin-1- yl)phenyl)amino)piperidine-2,6-dione | LCMS (ESI, m/z): 722.40 [M + H]⁺. 1H NMR (400 MHz, DMSO-d6) δ 12.12 (s, 1H), 10.77 (s, 1H), 7.98 (d, J = 8.7 Hz, 1H), 7.10-7.01 (m, 2H), 6.82 (t, J = 9.3 Hz, 1H), 6.51 (dd, J = 15.1, 2.6 Hz, 1H), 6.42 (dd, J = 8.8, 2.6 Hz, 1H), 5.79 (d, J = 7.7 Hz, 1H), 4.35-4.13 (m, 1H), 3.97 (d, J = 5.9 Hz, 2H), 3.89-3.74 (m, 2H), 3.65 (s, 2H), 3.34-3.24 (m, 3H), 3.13-3.01 (m, 1H), 2.92-2.79 (m, 6H), 2.79-2.66 (m, 1H), 2.62-2.57 (m, 3H), 2.48-2.37 (m, 4H), 2.14-2.05 (m, 1H), 2.03-1.82 (m, 5H), 1.75 (d, J = 12 Hz, 3H), 1.53-1.39 (m, 2H), 1.39-1.21 (m, 2H). |
| Example 207 | 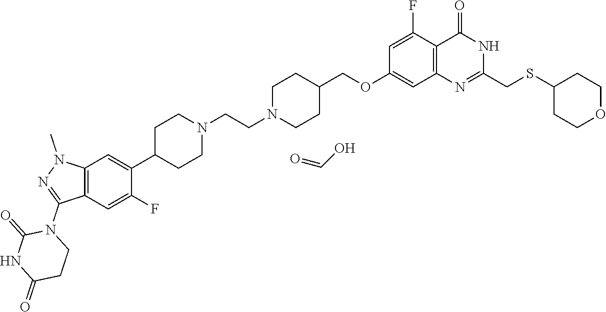 1-(5-fluoro-6-(1-(2-(4-(((5-fluoro-4-oxo-2-(((tetrahydro- 2H-pyran-4-yl)thio)methyl)-3,4-dihydroquinazolin-7- yl)oxy)methyl)piperidin-1-yl)ethyl)piperidin-4-yl)-1- methyl-1H-indazol-3-yl)dihydropyrimidine-2,4(1H,3H)- dione formate | LCMS (ESI, m/z): 779.35 [M + H]⁺. 1H NMR (400 MHz, DMSO-d6) δ 12.18 (s, 1H), 10.55 (s, 1H), 7.58 (d, J = 6.0 Hz, 1H), 7.37 (d, J = 10.9 Hz, 1H), 6.93- 6.84 (m, 2H), 4.02- 3.97 (m, 5H), 3.93- 3.88 (m, 2H), 3.86- 3.77 (m, 3H), 3.65 (s, 2H), 3.38-3.29 (m, 3H), 3.10-3.02 (m, 3H), 3.00-2.92 (m, 2H), 2.90-2.80 (m, 1H), 2.79-2.72 (m, 2H), 2.55-2.52 (m, 2H), 2.18-2.07 (m, 2H), 2.03-1.96 (m, 2H), 1.92-1.86 (m, 2H), 1.84-1.71 (m, 7H), 1.51-1.40 (m, 2H), 1.39-1.25 (m, 2H). |
| Example 208 | 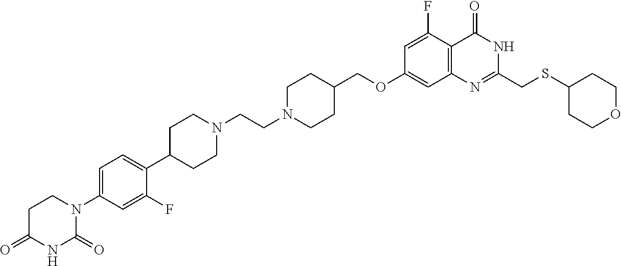 [SMILES: O=C1CCN(c2ccc(C3CCN(CCN4CCC(COc5cc(F)c6c(=O)[nH]c(CSC7CCOCC7)nc6c5)CC4)CC3)c(F)c2)C(=O)N1] 1-(3-fluoro-4-(1-(2-(4-(((5-fluoro-4-oxo-2-(((tetrahydro- 2H-pyran-4-yl)thio)methyl)-3,4-dihydroquinazolin-7- yl)oxy)methyl)piperidin-1-yl)ethyl)piperidin-4- yl)phenyl)dihydropyrimidine-2,4(1H,3H)-dione | LCMS (ESI, m/z): 725.30 [M + H]⁺. 1H NMR (300 MHz, DMSO-d6) δ 12.14 (br, 1H), 10.39 (s, 1H), 7.34 (t, J = 8.4 Hz, 1H), 7.23-7.08 (m, 2H), 6.93-6.82 (m, 2H), 4.10- 4.02 (m, 2H), 3.88- 3.76 (m, 4H), 3.81 (m, 2H), 3.62 (s, 2H), 3.33-3.20 (m, 2H), 3.15-2.86 (m, 5H), 2.80-2.65 (m, 3H), 2.55-2.40 (m, 4H), 2.15-1.84 (m, 6H), 1.80-1.65 (m, 7H), 1.55-1.21 (m, 4H). |
| Example 209 | 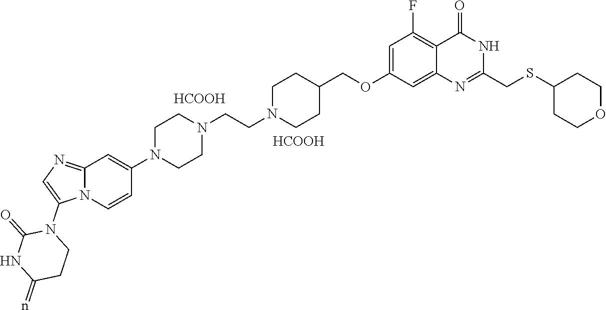 1-(7-(4-(2-(4-(((5-fluoro-4-oxo-2-(((tetrahydro-2H-pyran- 4-yl)thio)methyl)-3,4-dihydroquinazolin-7- yl)oxy)methyl)piperidin-1-yl)ethyl)piperazin-1- yl)imidazo[1,2-a]pyridin-3-yl)dihydropyrimidine- 2,4(1H,3H)-dione diformate | LCMS (ESI, m/z): 748.30 [M + H]⁺. 1H NMR (300 MHz, MeOD-d4) δ 8.48 (s, 2H), 8.11 (d, J = 7.6 Hz, 1H), 7.53 (s, 1H), 7.06 (d, J = 7.6 Hz, 1H), 6.96 (s, 1H), 6.87 (dd, J = 12.4 Hz, 2.0 Hz, 1H), 6.79 (s, 1H), 4.10 (d, J = 5.6Hz, 2H), 4.01- 3.89 (m, 4H), 3.73- 3.61 (m, 4H), 3.51- 3.42 (m, 6H), 3.39- 3.28 (m, 2H), 3.17- 3.01 (m, 3H), 3.00- 2.91 (m, 2H), 2.89- 2.78 (m, 2H), 2.78- 2.68 (m, 4H), 2.28- 2.19 (m, 1H), 2.15- 2.05 (m, 2H), 2.01- 1.87 (m, 2H), 1.83- 1.69 (m, 2H), 1.65- 1.51 (m, 2H). |
| Example 210 | 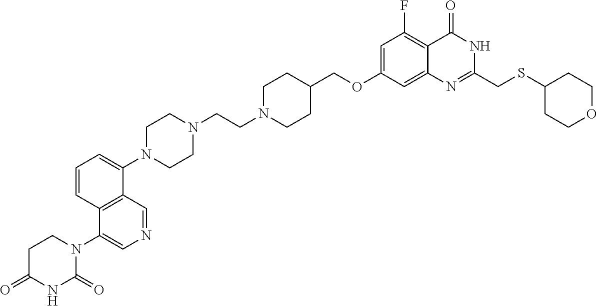 [SMILES: O=C1CCN(c2cncc3c(N4CCN(CCN5CCC(COc6cc(F)c7c(=O)[nH]c(CSC8CCOCC8)nc7c6)CC5)CC4)cccc23)C(=O)N1] 1-(8-(4-(2-(4-(((5-fluoro-4-oxo-2-(((tetrahydro-2H-pyran- 4-yl)thio)methyl)-3,4-dihydroquinazolin-7- yl)oxy)methyl)piperidin-1-yl)ethyl)piperazin-1- yl)isoquinolin-4-yl)dihydropyrimidine-2,4(1H,3H)-dione | LCMS (ESI, m/z): 759.30 [M + H]⁺. 1H NMR (400 MHz, DMSO-d6) δ 12.16 (br, 1H), 10.54 (s, 1H), 9.42 (s, 1H), 8.53 (s, 1H), 7.72 (t, J = 7.9 Hz, 1H), 7.62 (d, J = 8.4 Hz, 1H), 7.27 (d, J = 7.5 Hz, 1H), 6.93-6.84 (m, 2H), 4.05-3.96 (m, 2H), 3.95-3.85 (m, 1H), 3.85-3.78 (m, 2H), 3.76-3.67 (m, 1H), 3.65 (s, 2H), 3.32-3.30 (m, 3H), 3.20-3.01 (m, 5H), 3.00-2.90 (m, 3H), 2.81-2.66 (m, 5H), 2.62-2.44 (m, 3H), 2.03-1.82 (m, 4H), 1.82-1.72 (m, 3H), 1.52-1.39 (m, 2H), 1.38-1.28 (m, 2H). |
| Example 211 | 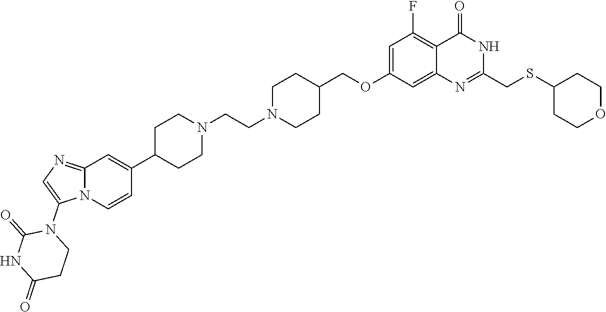 [SMILES: O=C1CCN(c2cnc3cc(C4CCN(CCN5CCC(COc6cc(F)c7c(=O)[nH]c(CSC8CCOCC8)nc7c6)CC5)CC4)ccn23)C(=O)N1] 1-(7-(1-(2-(4-(((5-fluoro-4-oxo-2-(((tetrahydro-2H-pyran- 4-yl)thio)methyl)-3,4-dihydroquinazolin-7- yl)oxy)methyl)piperidin-1-yl)ethyl)piperidin-4- yl)imidazo[1,2-a]pyridin-3-yl)dihydropyrimidine- 2,4(1H,3H)-dione | LCMS (ESI, m/z): 747.35 [M + H]⁺. 1H NMR (400 MHz, DMSO-d6) δ 12.16 (s, 1H), 10.63 (s, 1H), 8.21 (d, J = 8.0 Hz, 1H), 7.48 (s, 1H), 7.35 (s, 1H), 6.96-6.84 (m, 3H), 3.98 (d, J = 8.0 Hz, 2H), 3.87-3.74 (m, 4H), 3.62 (s, 2H), 3.39-3.33 (m, 1H), 3.11-2.97 (m, 3H), 2.92 (d, J = 12.0 Hz, 2H), 2.82 (t, J = 12.0 Hz, 2H), 2.51-2.42 (m, 3H), 2.11-2.01 (m, 2H), 2.01-1.85 (m, 4H), 1.83-1.61 (m, 7H), 1.52-1.38 (m, 2H), 1.37-1.22 (m, 5H). |
| Example 212 | 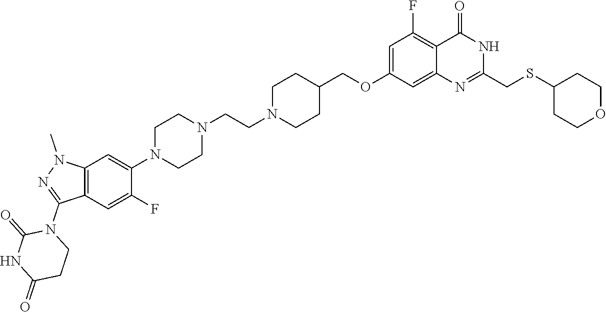 [SMILES: Cn1nc(N2CCC(=O)NC2=O)c2cc(F)c(N3CCN(CCN4CCC(COc5cc(F)c6c(=O)[nH]c(CSC7CCOCC7)nc6c5)CC4)CC3)cc21] 1-(5-fluoro-6-(4-(2-(4-(((5-fluoro-4-oxo-2-(((tetrahydro- 2H-pyran-4-yl)thio)methyl)-3,4-dihydroquinazolin-7- yl)oxy)methyl)piperidin-1-yl)ethyl)piperazin-1-yl)-1- methyl-1H-indazol-3-yl)dihydropyrimidine-2,4(1H,3H)- dione | LCMS (ESI, m/z): 780.25 [M + H]⁺. 1H NMR (300 MHz, DMSO-d6) δ 12.13 (br, 1H), 10.50 (s, 1H), 7.35 (d, J = 12.9 Hz, 1H), 7.08 (d, J = 7.1 Hz, 1H), 6.93- 6.82 (m, 2H), 4.08- 3.79 (m, 9H), 3.62 (s, 2H), 3.25-3.15 (m, 6H), 3.12-3.00 (m, 5H), 2.95 (d, J = 10.9 Hz, 2H), 2.74 (t, J = 6.6 Hz, 2H), 2.65- 2.57 (m, 4H), 2.05- 1.94 (m, 2H), 1.90- 1.83 (m, 2H), 1.80- 1.69 (m, 2H),, 1.50- 1.40 (m, 2H), 1.39- 1.20 (m, 2H). |

Examples 213-215 were synthesized according to the procedure described for the synthesis 3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutoxy)phenyl) amino)piperidine-2,6-dione (Example 81; steps C-E) using appropriate building blocks and modified reaction conditions (such as reagents, reagent ratio, temperature, and reaction time) and purification conditions as needed.

| Example | Structure | Analytical Data |
|---|---|---|
| Example 213 | 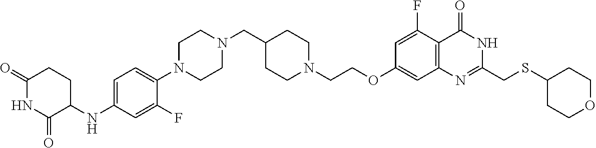 [SMILES: O=C1CCC(Nc2ccc(N3CCN(CC4CCN(CCOc5cc(F)c6c(=O)[nH]c(CSC7CCOCC7)nc6c5)CC4)CC3)c(F)c2)C(=O)N1] | LCMS (ESI, m/z): 740.30 [M + H]⁺. 1H NMR (400 MHz, DMSO-d6) δ 10.79 (s, 1H), 8.19 (s, 3H)HCOOH, 6.94- 6.78 (m, 3H), 6.54 (dd, J1 = 15.4 Hz, J2 = 6.4 Hz, 1H), 6.43 (d, |
| | 3-((3-fluoro-4-(4-((1-(2-((5-fluoro-4-oxo-2- | J = 8.8 Hz, 1H), 5.80 (s, |
| | (((tetrahydro-2H-pyran-4-yl)thio)methyl)-3,4- | 1H), 4.28 (s, 3H), 3.91- |
| | dihydroquinazolin-7-yl)oxy)ethyl)piperidin-4- | 3.76 (m, 2H), 3.64 (s, |
| | yl)methyl)piperazin-1-yl)phenyl)amino)piperidine- | 2H), 3.39-3.27 (m, |
| | 2,6-dione | 1H), 3.10-2.98 (m, |
| | | 3H), 2.92-2.80 (m, |
| | | 7H), 2.80-2.65 (m, |
| | | 1H), 2.60-2.55 (m, |
| | | 1H), 2.26-2.06 (m, |
| | | 6H), 1.98-1.78 (m, |
| | | 4H), 1.76-1.68 (m, |
| | | 2H), 1.62-1.42 (m, |
| | | 3H), 1.22-1.10 (m, |
| | | 3H). |
| Example 214 | 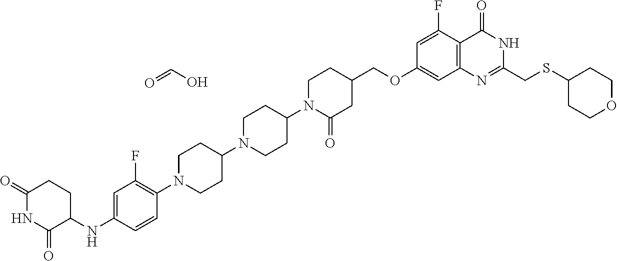 [SMILES: O=C1CCC(Nc2ccc(N3CCC(N4CCC(N5CCC(COc6cc(F)c7c(=O)[nH]c(CSC8CCOCC8)nc7c6)CC5=O)CC4)CC3)c(F)c2)C(=O)N1 ; O=CO] | LCMS (ESI, m/z): 808.45 [M + H]⁺. 1H NMR (400 MHz, DMSO-d6) δ 12.18 (s, 1H), 10.77 (s, 1H), 8.16 (s, 1H), 6.95-6.87 (m, 2H), 6.83 (t, J = 9.3 Hz, 1H), 6.54-6.45 (m, 1H), 6.44-6.37 (m, 1H), 5.79 (d, J = 7.6 Hz, 1H), 4.32-4.20 (m, 2H), 4.03 (d, J = 6.7 Hz, 2H), 3.86-3.77 (m, 2H), 3.62 (s, 2H), 3.39- 3.35 (m, 2H), 3.30- 3.28 (m, 1H), 3.20- |
| | 3-((3-fluoro-4-(4-(((5-fluoro-4-oxo-2-(((tetrahydro- | 3.13 (m, 3H), 3.10- |
| | 2H-pyran-4-yl)thio)methyl)-3,4-dihydroquinazolin-7- | 2.95 (m, 3H), 2.79- |
| | yl)oxy)methyl)-2-oxo-[1,4′:1′,4″-terpiperidin]-1″- | 2.66 (m, 1H), 2.61- |
| | yl)phenyl)amino)piperidine-2,6-dione formate. | 2.53 (m, 3H), 2.49- |
| | | 2.37 (m, 2H), 2.34- |
| | | 2.23 (m, 3H), 2.18- |
| | | 2.04 (m, 2H), 2.05- |
| | | 1.95 (d, J = 12.3 Hz, |
| | | 1H), 1.93-1.85 (m, |
| | | 3H), 1.83-1.63 (m, |
| | | 4H), 1.63-1.39 (m, |
| | | 7H) |
| Example 215 | 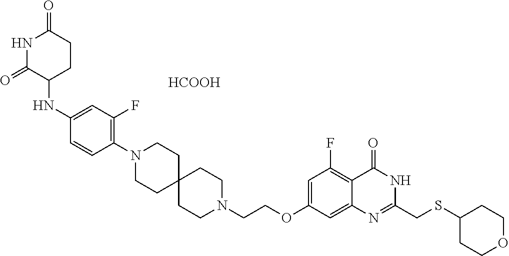 [SMILES: O=C1CCC(Nc2ccc(N3CCC4(CCN(CCOc5cc(F)c6c(=O)[nH]c(CSC7CCOCC7)nc6c5)CC4)CC3)c(F)c2)C(=O)N1 ; "HCOOH"] | LCMS (ESI, m/z): 711.30 [M + H]⁺. 1H NMR (300 MHz, DMSO-d6) δ 12.20 (br, 1H), 10.80 (s, 1H), 8.20 (s, 1H), 6.99-6.78 (m, 3H), 6.52 (dd, J1 = 14.8 Hz, J2 = 2.0 Hz, 1H), 6.42 (dd, J1 = 8.4 Hz, J2 = 2.0 Hz, 1H), 5.78 (d, J = 7.6 Hz, 1H), 4.25(s, 3H), 4.11-3.79 (m, 7H), 3.65 (s, 2H), 3.39-3.29 (m, 2H), 3.11-3.03 (m,1H), 2.85-2.61 (m,7H), 2.65-2.53 |
| | 3-((3-fluoro-4-(9-(2-((5-fluoro-4-oxo-2-(((tetrahydro- | (m,1H), 2.13-2.07 |
| | 2H-pyran-4-yl)thio)methyl)-3,4-dihydroquinazolin-7- | (m,1H), 1.97-1.79 |
| | yl)oxy)ethyl)-3,9-diazaspiro[5.5]undecan-3- | (m,3H), 1.41-1.59 |
| | yl)phenyl)amino)piperidine-2,6-dione | (m,9H). |

Example 216 was synthesized according to the procedure described for the synthesis 3-((4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione (Example 82) using appropriate building blocks and modified reaction conditions (such as reagents, reagent ratio, temperature, and reaction time) and purification conditions as needed.

| Example | Structure | Analytical Data |
|---|---|---|
| Example 216 | 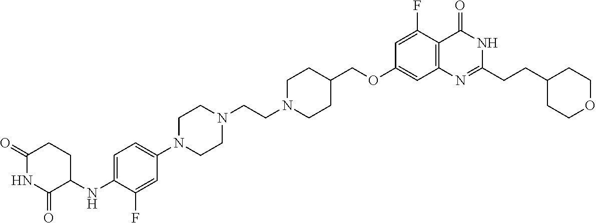 [SMILES: O=C1CCC(Nc2ccc(N3CCN(CCN4CCC(COc5cc(F)c6c(=O)[nH]c(CCC7CCOCC7)nc6c5)CC4)CC3)cc2F)C(=O)N1] | LCMS (ESI, m/z): 722.35 [M + H]⁺. 1H NMR (400 MHz, DMSO-d6) δ 12.04 (s, 1H), 10.80 (s, 1H), 6.87-6.70 (m, 4H), 6.62-6.55 (m, 1H), 5.06 (d, J = 6.0 Hz, 1H), 4.32-4.21 (m, 1H), 3.97 (d, J = 5.9 Hz, 2H), 3.88-3.79 (m, 2H), 3.30-3.20 (m, 3H), 3.00-2.87 (m, 6H), 2.81-2.70 |
| | 3-((2-fluoro-4-(4-(2-(4-(((5-fluoro-4-oxo-2-(2- | (m, 1H), 2.59-2.54 |
| | (tetrahydro-2H-pyran-4-yl)ethyl)-3,4- | (m, 4H), 2.48-4.40 |
| | dihydroquinazolin-7-yl)oxy)methyl)piperidin-1- | (m, 4H), 2.14-2.05 |
| | yl)ethyl)piperazin-1-yl)phenyl)amino)piperidine-2,6- | (m, 1H), 2.04-1.89 |
| | dione | (m, 3H), 1.80-1.56 |
| | | (m, 7H), 1.55-1.45 |
| | | (m, 1H), 1.36-1.11 |
| | | (m, 6H). |

Examples 217-220 were synthesized according to the procedure described for the synthesis 3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1I-yl)azetidin-1-yl)piperidin-1-yl)phenyl)amino)piperidine-2,6-dione (Example 88) using appropriate building blocks and modified reaction conditions (such as reagents, reagent ratio, temperature, and reaction time) and purification conditions as needed.

| Example | Structure | Analytical Data |
|---|---|---|
| Example 217 | 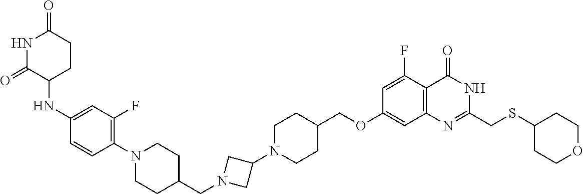 [SMILES: O=C1CCC(Nc2ccc(N3CCC(CN4CC(N5CCC(COc6cc(F)c7c(=O)[nH]c(CSC8CCOCC8)nc7c6)CC5)C4)CC3)c(F)c2)C(=O)N1] | LCMS (ESI, m/z): 780.30 [M + H]⁺. 1H NMR (400 MHz, DMSO-d6) δ 12.15 (br, 1H), 10.76 (s, 1H), 8.20 (s, 1H) HCOOH, 6.91- 6.85 (m, 2H), 6.82 (t, J = 12 Hz, 1H), 6.50 |
| | 3-((3-fluoro-4-(4-((3-(4-(((5-fluoro-4-oxo-2-(((tetrahydro- | (dd, J = 15.0, 2.6 |
| | 2H-pyran-4-yl)thio)methyl)-3,4-dihydroquinazolin-7- | Hz, 1H), 6.41 |
| | yl)oxy)methyl)piperidin-1-yl)azetidin-1-yl)methyl)piperidin- | (dd, J = 8.7, 2.6 |
| | 1-yl)phenyl)amino)piperidine-2,6-dione formate | Hz, 1H), 5.77 (d, |
| | | J = 7.6 Hz, 1H), |
| | | 4.30-4.20 (m, |
| | | 1H), 3.98 (d, J = |
| | | 5.7 Hz, 2H), 3.87- |
| | | 3.77 (m, 2H), |
| | | 3.62 (s, 2H), 3.38- |
| | | 3.28 (m, 2H), |
| | | 3.12-3.00 (m, |
| | | 3H), 2.86 (s, |
| | | 3H), 2.80-2.67 |
| | | (m, 3H), 2.60- |
| | | 2.50 (m, 3H), |
| | | 2.45-2.40(m, |
| | | 2H), 2.39-2.31 |
| | | (m, 2H), 2.09- |
| | | 2.04 (m, 1H), |
| | | 1.93-1.82 (m, |
| | | 3H), 1.81-1.66 |
| | | (m, 7H), 1.53- |
| | | 1.40 (m, 2H), |
| | | 1.39-1.18 (m, |
| | | 5H). |
| Example 218 | 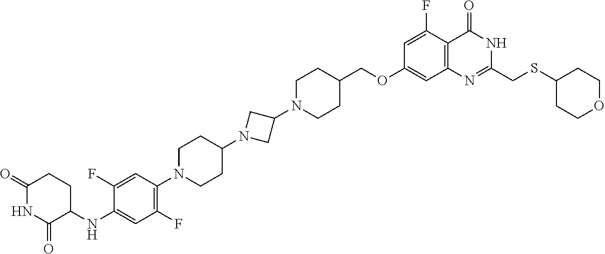 [SMILES: O=C1CCC(Nc2cc(F)c(N3CCC(N4CC(N5CCC(COc6cc(F)c7c(=O)[nH]c(CSC8CCOCC8)nc7c6)CC5)C4)CC3)cc2F)C(=O)N1] | LCMS (ESI, m/z): 784.30 [M + H]⁺. 1H NMR (400 MHz, DMSO-d6) δ 12.15 (s, 1H), 10.79 (s, 1H), 6.89-6.79 (m, 3H), 6.78-6.63 (m, 1H), 5.46 (d, J = 4.0 Hz, 1H), 4.39-4.29 (m, 1H), 3.98 (d, J = 4.0 Hz, 2H), 3.85- 3.80 (m, 2H), 3.62 (s, 2H), 3.41- |
| | 3-((2,5-difluoro-4-(4-(3-(4-(((5-fluoro-4-oxo-2-(((tetrahydro- | 3.39 (m, 2H), |
| | 2H-pyran-4-yl)thio)methyl)-3,4-dihydroquinazolin-7- | 3.36-3.32 (m, |
| | yl)oxy)methyl)piperidin-1-yl)azetidin-1-yl)piperidin-1- | 3H), 3.10-3.03 |
| | yl)phenyl)amino)piperidine-2,6-dione | (m, 2H), 2.82- |
| | | 2.69 (m, 6H), |
| | | 2.62-2.53 (m, |
| | | 3H), 2.13-1.97 |
| | | (m, 3H), 1.94- |
| | | 1.86 (m, 2H), |
| | | 1.81-1.65 (m, |
| | | 7H), 1.51-1.39 |
| | | (m, 2H), 1.35- |
| | | 1.22 (m, 4H). |
| Example 219 | 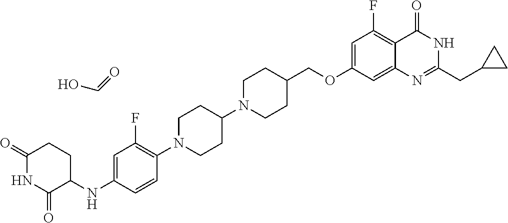 [SMILES: O=C1CCC(Nc2ccc(N3CCC(N4CCC(COc5cc(F)c6c(=O)[nH]c(CC7CC7)nc6c5)CC4)CC3)c(F)c2)C(=O)N1 ; O=CO] | LCMS (ESI, m/z): 635.30 [M + H]⁺. 1H NMR (400 MHz, DMSO-d6) δ 12.01 (s, 1H), 10.76 (s, 1H), 8.31 (s, 1H), 6.91- 6.78 (m, 3H), 6.55-6.37 (m, 2H), 5.78 (d, J = 7.7 Hz, 1H), 4.30- 4.19 (m, 1H), 3.98 (d, J = 5.7 |
| | 3-((4-(4-(((2-(cyclopropylmethyl)-5-fluoro-4-oxo-3,4- | Hz, 2H), 3.17 (d, |
| | dihydroquinazolin-7-yl)oxy)methyl)-[1,4′-bipiperidin]-1′-yl)- | J = 10.7 Hz, 2H), |
| | 3-fluorophenyl)amino)piperidine-2,6-dione formate | 2.93 (d, J = 10.5 |
| | | Hz, 2H), 2.61- |
| | | 2.53 (m, 3H), |
| | | 2.48-2.39 (m, |
| | | 3H), 2.28-2.14 |
| | | (m, 2H), 2.14- |
| | | 2.03 (m, 1H), |
| | | 2.00-1.67 (m, |
| | | 6H), 1.67-1.50 |
| | | (m, 2H), 1.38- |
| | | 1.10 (m, 4H), |
| | | 0.53-0.44 (m, |
| | | 2H), 0.33-0.21 |
| | | (m, 2H). |
| Example 220 | 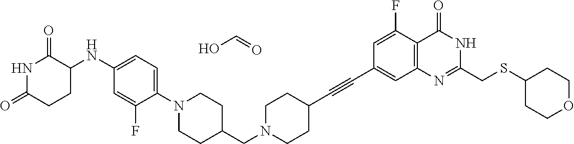 [SMILES: O=C1CCC(Nc2ccc(N3CCC(CN4CCC(C#Cc5cc(F)c6c(=O)[nH]c(CSC7CCOCC7)nc6c5)CC4)CC3)c(F)c2)C(=O)N1 ; O=CO] | LCMS (ESI, m/z): 719.30 [M + H]⁺. 1H NMR (300 MHz, DMSO-d6) δ 12.39 (br, 1H), 10.75 (s, 1H), 8.17 (s, 1H), 7.37 (s, 1H), 7.26- |
| | 3-((3-fluoro-4-(4-((4-((5-fluoro-4-oxo-2-(((tetrahydro-2H- | 7.16 (m, 1H), |
| | pyran-4-yl)thio)methyl)-3,4-dihydroquinazolin-7- | 6.83 (t, J = 9.3 |
| | yl)ethynyl)piperidin-1-yl)methyl)piperidin-1- | Hz, 1H), 6.56- |
| | yl)phenyl)amino)piperidine-2,6-dione formate | 6.37 (m, 2H), |
| | | 5.76 (d, J = 7.5 |
| | | Hz, 1H), 4.31- |
| | | 4.18 (m, 1H), |
| | | 3.86-3.78 (m, |
| | | 2H), 3.62 (s, |
| | | 2H), 3.14-3.03 |
| | | (m, 4H), 2.77- |
| | | 2.64 (m, 4H), |
| | | 2.62-2.34 (m, |
| | | 3H), 2.23-2.03 |
| | | (m, 5H), 1.97- |
| | | 1.81 (m, 5H), |
| | | 1.80-1.53 (m, |
| | | 6H), 1.51-1.38 |
| | | (m, 2H), 1.31- |
| | | 1.18 (m, 2H). |

Examples 221-225 were synthesized according to the procedure described for the synthesis 3-((4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione (Example 112) using appropriate building blocks and modified reaction conditions (such as reagents, reagent ratio, temperature, and reaction time) and purification conditions as needed.

| Example | Structure | Analytical Data |
|---|---|---|
| Example 221 | 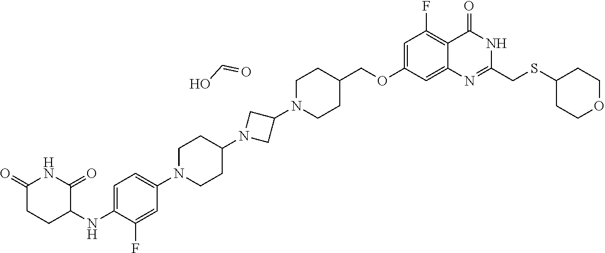 [SMILES: O=C1CCC(Nc2ccc(N3CCC(N4CC(N5CCC(COc6cc(F)c7c(=O)[nH]c(CSC8CCOCC8)nc7c6)CC5)C4)CC3)cc2F)C(=O)N1 ; O=CO] | LCMS (ESI, m/z): 766.40 [M + H]⁺. 1H NMR (400 MHz, DMSO-d6) δ 12.17 (br, 1H), 10.79 (s, 1H), 8.24 (s, 1H), 6.92-6.84 (m, 2H), 6.78-6.69 (m, 2H), 6.58 (dd, J = 8.9, 2.6 Hz, 1H), 5.04 (dd, J = 7.6, 2.2 Hz, 1H), 4.31- 4.20 (m, 1H), 3.98 (d, J = 5.8 Hz, 2H), 3.87-3.77 (m, 2H), 3.62 (s, 2H), |
| | 3-((2-fluoro-4-(4-(3-(4-(((5-fluoro-4-oxo-2-(((tetrahydro- | 3.36-3.31 (m, 5H), |
| | 2H-pyran-4-yl)thio)methyl)-3,4-dihydroquinazolin-7- | 3.09-3.02 (m, |
| | yl)oxy)methyl)piperidin-1-yl)azetidin-1-yl)piperidin-1- | 1H), 2.86-2.69 |
| | yl)phenyl)amino)piperidine-2,6-dione formate | (m, 6H), 2.65- |
| | | 2.54 (m, 4H), 2.15- |
| | | 2.04 (m, 2H), |
| | | 2.03-1.84 (m, |
| | | 3H), 1.82-1.64 |
| | | (m, 7H), 1.52- |
| | | 1.38 (m, 2H), 1.33- |
| | | 1.19 (m, 4H). |
| Example 222 | 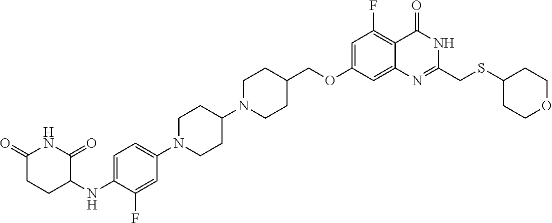 [SMILES: O=C1CCC(Nc2ccc(N3CCC(N4CCC(COc5cc(F)c6c(=O)[nH]c(CSC7CCOCC7)nc6c5)CC4)CC3)cc2F)C(=O)N1] | LCMS (ESI, m/z): 711.30 [M + H]⁺. 1H NMR (400 MHz, DMSO-d6) δ 12.15 (s, 1H), 10.79 (s, 1H), 6.92-6.84 (m, 2H), 6.79- 6.69 (m, 2H), 6.61- 6.58 (m, 1H), 5.05 (d, J = 6.8 Hz, 1H), 4.29-4.23 (m, 1H), 3.98 (d, J = 8.0 Hz, 2H), 3.85-3.80 (m, 2H), |
| | 3-((2-fluoro-4-(4-(((5-fluoro-4-oxo-2-(((tetrahydro-2H- | 3.62 (s, 2H), 3.52 |
| | pyran-4-yl)thio)methyl)-3,4-dihydroquinazolin-7- | (d, J = 12.0 Hz, |
| | yl)oxy)methyl)-[1,4′-bipiperidin]-1′- | 2H), 3.37-3.32 |
| | yl)phenyl)amino)piperidine-2,6-dione | (m, 2H), 3.10- |
| | | 3.03 (m, 1H), 2.99- |
| | | 2.85 (m, 2H), |
| | | 2.79-2.70 (m, 1H), |
| | | 2.61-2.53 (m, 4H), |
| | | 2.22-2.06 (m, 3H), |
| | | 2.02-1.95 (m, |
| | | 1H), 1.93-1.87 |
| | | (m, 2H), 1.84- |
| | | 1.71 (m, 5H), 1.61- |
| | | 1.51 (m, 2H) 1.49- |
| | | 1.38 (m, 2H), |
| | | 1.36-1.22 (m, |
| | | 2H). |
| Example 223 | 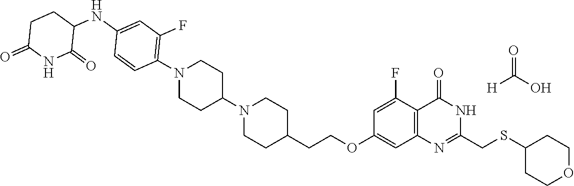 [SMILES: O=C1CCC(Nc2ccc(N3CCC(N4CCC(CCOc5cc(F)c6c(=O)[nH]c(CSC7CCOCC7)nc6c5)CC4)CC3)c(F)c2)C(=O)N1 ; O=CO] | LCMS (ESI, m/z): 725.30 [M + H]⁺. 1H NMR (400 MHz, DMSO-d6) δ 12.15 (br, 1H), 10.76 (s, 1H), 8.20 (s, 1H), 6.90 (s, 1H), 6.87- 6.78 (m, 2H), 6.50 (d, J = 12 Hz, 1H), 6.41 (s, J = 12 Hz, 1H), 5.78 (d, J = 7.6 Hz, 1H), 4.28- |
| | 3-((3-fluoro-4-(4-(2-((5-fluoro-4-oxo-2-(((tetrahydro-2H- | 4.21 (m, 1H), 4.16 |
| | pyran-4-yl)thio)methyl)-3,4-dihydroquinazolin-7- | (t, J = 6.5 Hz, 2H), |
| | yl)oxy)ethyl)-[1,4′-bipiperidin]-1′- | 3.82 (dt, J = 11.6, |
| | yl)phenyl)amino)piperidine-2,6-dione | 3.8 Hz, 2H), 3.62 |
| | | (s, 2H), 3.37-3.29 |
| | | m, 3H), 3.17 (d, J = |
| | | 10.8 Hz, 2H), 3.13- |
| | | 3.01 (m, 1H), |
| | | 2.94 (d, J = 10.7 |
| | | Hz, 2H), 2.79- |
| | | 2.67 m, 1H), 2.61- |
| | | 2.54 (m, 2H), 2.40- |
| | | 2.30(m, 1H), 2.23- |
| | | 2.12 (m, 2H), 2.13- |
| | | 2.04 (m, 1H), |
| | | 1.92-1.50 (m, |
| | | 9H), 1.49-1.39 (m, |
| | | 3H), 1.23-1.10 (m, |
| | | 2H). |
| Example 224 | 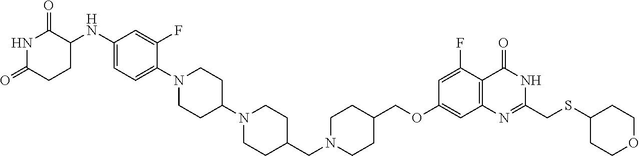 [SMILES: O=C1CCC(Nc2ccc(N3CCC(N4CCC(CN5CCC(COc6cc(F)c7c(=O)[nH]c(CSC8CCOCC8)nc7c6)CC5)CC4)CC3)c(F)c2)C(=O)N1] | LCMS (ESI, m/z): 808.45 [M + H]⁺. 1H NMR (300 MHz, DMSO-d6) δ 6.89- 6.75 (m, 3H), 6.49 (dd, J = 15.0, 2.5 Hz, 1H), 6.40 (dd, J = 8.7, 2.5 Hz, 1H), 5.80-5.71 (m, 1H), 4.30-4.20 |
| | 3-((3-fluoro-4-(4-((4-(((5-fluoro-4-oxo-2-(((tetrahydro- | (m, 1H), 3.96 (d, |
| | 2H-pyran-4-yl)thio)methyl)-3,4-dihydroquinazolin-7- | J = 5.7 Hz, 2H), 3.81 |
| | yl)oxy)methyl)piperidin-1-yl)methyl)-[1,4′-bipiperidin]- | (dt, J = 11.6, 3.7 |
| | 1′-yl)phenyl)amino)piperidine-2,6-dione. | Hz, 2H), 3.60 (s, |
| | | 2H), 3.51-3.45 (m, |
| | | 2H), 3.20 (d, J = |
| | | 5.7 Hz, 2H), 3.15- |
| | | 2.98 (m, 1H), 2.90- |
| | | 2.79 (m, 4H), |
| | | 2.80-2.69 (m, |
| | | 1H), 2.64-2.50 |
| | | (m, 2H), 2.32- |
| | | 2.20 (m, 1H), 2.18- |
| | | 2.05 (m, 5H), |
| | | 1.94-1.81 (m, |
| | | 5H), 1.80-1.65 |
| | | (m, 8H), 1.63- |
| | | 1.40 (m, 5H), 1.39- |
| | | 1.25 (m, 2H), |
| | | 1.15-1.00 (m, |
| | | 2H). |
| Example 225 | 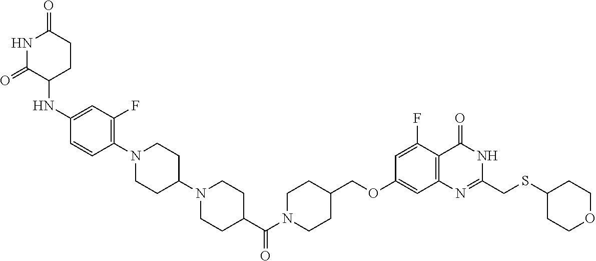 [SMILES: O=C1CCC(Nc2ccc(N3CCC(N4CCC(C(=O)N5CCC(COc6cc(F)c7c(=O)[nH]c(CSC8CCOCC8)nc7c6)CC5)CC4)CC3)c(F)c2)C(=O)N1] | LCMS (ESI, m/z): 822.40 [M + H]⁺. 1H NMR (300 MHz, DMSO-d6) δ 10.77 (br, 1H), 6.94- 6.77 (m, 3H), 6.50 (dd, J = 15.0, 2.6 Hz, 1H), 6.41 (dd, J = 8.8, 2.5 Hz, 1H), 5.77 (d, J = 7.6 Hz, 1H), 4.43 (d, J = 12.9 Hz, 1H), 4.25 (dt, J = 11.7, 6.7 Hz, 1H), 4.01- 3.92 (m, 3H), 3.90- 3.78 (m, 2H), |
| | 3-((3-fluoro-4-(4-(4-(((5-fluoro-4-oxo-2-(((tetrahydro-2H- | 3.65 (s, 2H), 3.30- |
| | pyran-4-yl)thio)methyl)-3,4-dihydroquinazolin-7- | 3.21 (m, 4H), 3.21- |
| | yl)oxy)methyl)piperidine-1-carbonyl)-[1,4′-bipiperidin]- | 3.06 (m, 2H), |
| | 1′-yl)phenyl)amino)piperidine-2,6-dione | 3.08-3.01 (m, |
| | | 2H), 2.89 (d, J = |
| | | 10.5 Hz, 2H), 2.79- |
| | | 2.70 (m, 1H), |
| | | 2.63-2.53 (m, |
| | | 3H), 2.39-2.19 |
| | | (m, 3H), 2.23- |
| | | 1.92 (m, 2H), 1.94- |
| | | 1.71 (m, 7H), |
| | | 1.73-1.48 (m, 8H), |
| | | 1.32-1.02 (m, |
| | | 2H). |

Example 226 was synthesized according to the procedure described for the synthesis 3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-1-yl)phenyl)amino)piperidine-2,6-dione (Example 118) using appropriate building blocks and modified reaction conditions (such as reagents, reagent ratio, temperature, and reaction time) and purification conditions as needed.

| Example | Structure | Analytical Data |
|---|---|---|
| Example 226 | 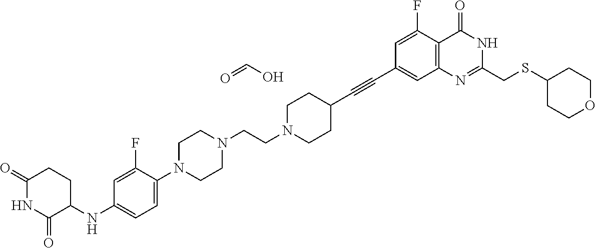 | LCMS (ESI, m/z): 734.20 [M + H]⁺ . H- NMR: 1H NMR (400 MHz, DMSO-d6) δ 10.77 (s, 1H), 8.24(s, 1H), 7.38 (s, 1H), 7.22 (d, J = 11.0 Hz, 1H), 6.82 (t, J = 9.4 Hz, 1H), 6.55 - 6.39 (m, 2H), 5.79 (d, J = 7.7 Hz, 1H), 4.31 - 4.20 (m, 1H), 3.85 - 3.79 (m, 2H), 3.63 (s, 2H), 3.33 - 3.31 (m, 2H), 3.11 - 3.01 (m, 2H), 2.89 - 2.80 (m, 4H), |
| | 3-((3-fluoro-4-(4-(2-(4-((5-fluoro-4-oxo-2- | 2.78 - 2.67 (m, 4H), |
| | (((tetrahydro-2H-pyran-4-yl)thio)methyl)-3,4- | 2.61 - 2.59 (m, 1H), |
| | dihydroquinazolin-7-yl)ethynyl)piperidin-1- | 2.58 - 2.54 (m, 3H), |
| | yl)ethyl)piperazin-1-yl)phenyl)amino)piperidine-2,6- | 2.49 - 2.45 (m, 4H), |
| | dione formate | 2.29 - 2.18 (m, 2H), |
| | | 2.11 - 2.06 (m, 1H), |
| | | 1.95 - 1.80 (m, 5H), |
| | | 1.70 - 1.58 (m, 2H), |
| | | 1.50 - 1.34 (m, 2H). |

Example 227 was synthesized according to the procedure described for steps E-G of the synthesis 3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione (Example 136) using appropriate building blocks and modified reaction conditions (such as reagents, reagent ratio, temperature, and reaction time) and purification conditions as needed.

| Example | Structure | Analytical Data |
|---|---|---|
| Example 227 | 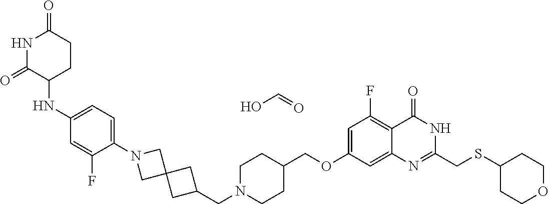 [SMILES: O=C1CCC(Nc2ccc(N3CC4(CC(CN5CCC(COc6cc(F)c7c(=O)[nH]c(CSC8CCOCC8)nc7c6)CC5)C4)C3)c(F)c2)C(=O)N1 ; O=CO] | LCMS (ESI, m/z): 737.30 [M + H]⁺. 1H NMR (400 MHz, DMSO-d6) δ 10.78 (s, 1H), 8.47 (s, 1H), 6.97- 6.86 (m, 2H), 6.64- 6.50 (m, 2H), 6.39 (dd, J = 8.6, 2.6 Hz, 1H), 5.50- 5.40 (m, 1H), 4.60-4.46 (m, 1H), 4.25-4.13 (m, 3H), 3.91-3.75 (m, 9H), 3.65 (s, 2H), 3.55-3.49 (m, 2H), 3.37-3.29 (m, 2H), 3.13-3.01 (m, 1H), |
| | 3-((3-fluoro-4-(6-((4-(((5-fluoro-4-oxo-2- | 2.97 (s, 2H), 2.77-2.67 |
| | (((tetrahydro-2H-pyran-4-yl)thio)methyl)-3,4- | (m, 1H), 2.64-2.57 (m, |
| | dihydroquinazolin-7-yl)oxy)methyl)piperidin-1- | 1H), 2.22-1.70 (m, |
| | yl)methyl)-2-azaspiro[3.3]heptan-2- | 12H), 1.53-1.39 (m, |
| | yl)phenyl)amino)piperidine-2,6-dione formate | 2H). |

Examples 228-231 were synthesized according to the procedure described for the synthesis afford 3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)phenyl)amino)piperidine-2,6-dione (Example 144) using appropriate building blocks and modified reaction conditions (such as reagents, reagent ratio, temperature, and reaction time) and purification conditions as needed.

| Example | Structure | Analytical Data |
|---|---|---|
| Example 228 | 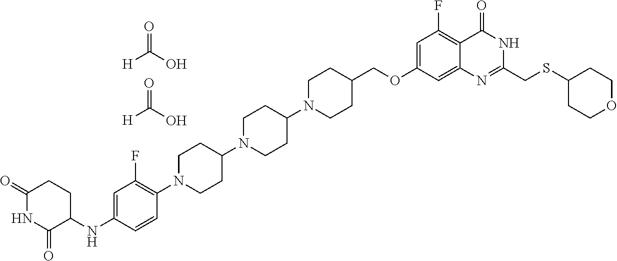 | LCMS (ESI, m/z): 794.35 [M + H]⁺. 1H NMR (400 MHz, DMSO-d6) δ 12.17 (s, 1H), 10.76 (s, 1H), 8.22 (s, 2H, HCOOH), 6.99- 6.73 (m, 3H), 6.56- 6.35 (m, 2H), 5.78 (d, J = 7.6 Hz, 1H), 4.31-4.21 (m, 1H), 3.98 (d, J = 5.8 Hz, 2H), 3.85-3.79 (m, 2H), 3.62 (s, 2H), 3.41-3.28 (m, 3H), 3.16 (d, J = 10.7 Hz, |
| | 3-((3-fluoro-4-(4-(((5-fluoro-4-oxo-2-(((tetrahydro-2H- | 2H), 3.11-3.05 (m, |
| | pyran-4-yl)thio)methyl)-3,4-dihydroquinazolin-7- | 1H), 3.01-2.89 (m, |
| | yl)oxy)methyl)-[1,4′:1′,4″-terpiperidin]-1″- | 4H), 2.81-2.66 (m, |
| | yl)phenyl)amino)piperidine-2,6-dione diformate | 2H), 2.60-2.54 (m, |
| | | 2H), 2.35-2.14 (m, |
| | | 6H), 2.13-2.06 (m, |
| | | 1H), 1.94-1.85 (m, |
| | | 2H), 1.85-1.67 (m, |
| | | 7H), 1.64-1.51 (m, |
| | | 2H), 1.50-1.37 (m, |
| | | 4H), 1.31-1.23 (m, |
| | | 2H). |
| Example 229 | 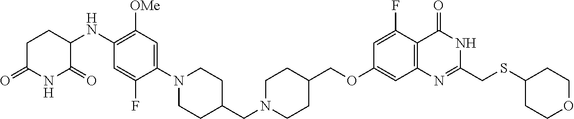 [SMILES: COc1cc(N2CCC(CN3CCC(COc4cc(F)c5c(=O)[nH]c(CSC6CCOCC6)nc5c4)CC3)CC2)c(F)cc1NC1CCC(=O)NC1=O] | LCMS (ESI, m/z): 755.35 [M + H]⁺. 1H NMR (400 MHz, DMSO-d6) δ 12.14 (br, 1H), 10.84 (s, 1H), 6.92-6.84 (m, 2H), 6.60 (d, J = 8.1 Hz, 1H), 6.53 (d, J = |
| | 3-((5-fluoro-4-(4-((4-(((5-fluoro-4-oxo-2-(((tetrahydro- | 14.1 Hz, 1H), 5.07 |
| | 2H-pyran-4-yl)thio)methyl)-3,4-dihydroquinazolin-7- | (d, J = 6.7 Hz, 1H), |
| | yl)oxy)methyl)piperidin-1-yl)methyl)piperidin-1-yl)-2- | 4.30-4.20 (m, 1H), |
| | methoxyphenyl)amino)piperidine-2,6-dione | 3.99 (d, J = 5.8 Hz, |
| | | 2H), 3.82-3.78 (m, |
| | | 5H), 3.62 (s, 2H), |
| | | 3.38-3.28 (m, 2H), |
| | | 3.20-3.00 (m, 3H), |
| | | 2.89-2.75 (m, 3H), |
| | | 2.70-2.55 (m, 3H), |
| | | 2.20-2.15 (m, 3H), |
| | | 1.95-1.85 (m, 5H), |
| | | 1.80-1.75 (m, 5H), |
| | | 1.70-1.55 (m, 1H), |
| | | 1.53-1.41 (m, 2H), |
| | | 1.44-1.18 (m, 4H). |
| Example 230 | 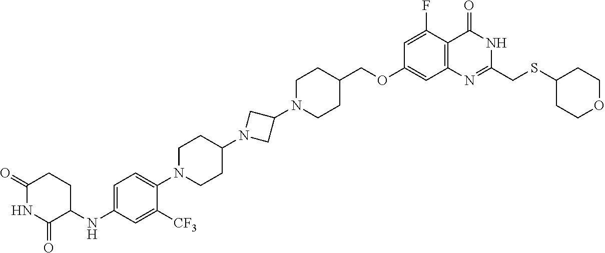 [SMILES: O=C1CCC(Nc2ccc(N3CCC(N4CC(N5CCC(COc6cc(F)c7c(=O)[nH]c(CSC8CCOCC8)nc7c6)CC5)C4)CC3)c(C(F)(F)F)c2)C(=O)N1] | LCMS (ESI, m/z): 816.35 [M + H]⁺. 1H NMR (400 MHz, DMSO-d6) δ 12.14 (br, 1H), 10.78 (s, 1H), 7.27 (d, J = 8.6 Hz, 1H), 6.93-6.83 (m, 4H), 6.16 (d, J = 7.9 Hz, 1H), 4.41- 4.32 (m, 1H), 3.98 (d, J = 5.7 Hz, 2H), 3.90-3.79 (m, 2H), 3.62 (s, 2H), 3.10- 3.00 (m, 1H), 2.86- 2.67 (m, 9H), 2.64- 2.55 (m, 3H), 2.12- |
| | 3-((4-(4-(3-(4-(((5-fluoro-4-oxo-2-(((tetrahydro-2H- | 2.03 (m, 2H), 1.93- |
| | pyran-4-yl)thio)methyl)-3,4-dihydroquinazolin-7- | 1.85 (m, 3H), 1.80- |
| | yl)oxy)methyl)piperidin-1-yl)azetidin-1-yl)piperidin-1- | 1.71 (m, 5H), 1.70- |
| | yl)-3-(trifluoromethyl)phenyl)amino)piperidine-2,6-dione | 1.62 (m, 2H), 1.50- |
| | | 1.40 (m, 2H), 1.33- |
| | | 1.21 (m, 7H). |
| Example 231 | 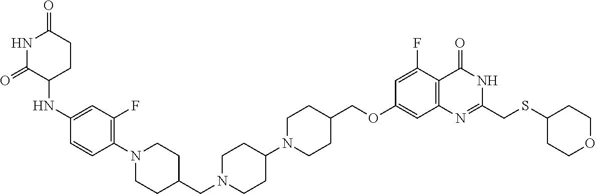 [SMILES: O=C1CCC(Nc2ccc(N3CCC(CN4CCC(N5CCC(COc6cc(F)c7c(=O)[nH]c(CSC8CCOCC8)nc7c6)CC5)CC4)CC3)c(F)c2)C(=O)N1] | LCMS (ESI, m/z): 808.35 [M + H]⁺. 1H NMR (400 MHz, DMSO-d6) δ 12.15 (s, 1H), 10.75 (s, 1H), 6.91-6.78 (m, 3H), 6.48 (d, J = 2.4 Hz, 1H), 6.44-6.38 (m, 1H), 5.76 (d, J = 8.0 Hz, 1H), 4.30- 4.19 (m, 1H), 3.98 (d, J = 4.0 Hz, 2H), |
| | 3-((3-fluoro-4-(4-((4-(((5-fluoro-4-oxo-2-(((tetrahydro- | 3.87-3.77 (m, 2H), |
| | 2H-pyran-4-yl)thio)methyl)-3,4-dihydroquinazolin-7- | 3.62 (s, 2H), 3.38- |
| | yl)oxy)methyl)-[1,4′-bipiperidin]-1′-yl)methyl)piperidin- | 3.25 (m, 2H), 3.14- |
| | 1-yl)phenyl)amino)piperidine-2,6-dione | 3.00 (m, 3H), 3.00- |
| | | 2.81 (m, 3H), 2.80- |
| | | 2.65 (m, 2H), 2.63- |
| | | 2.52 (m, 2H), 2.25- |
| | | 2.03 (m, 5H), 1.95- |
| | | 1.65 (m, 12H), 1.60- |
| | | 1.37 (m, 6H), 1.35- |
| | | 1.14 (m, 5H). |

Examples 232-237 were synthesized according to the procedure described for the synthesis 3-((3-fluoro-4-(4-(3-(4-(((5-fluoro-4-oxo-2-(2-(tetrahydro-2H-pyran-4-yl)ethyl)-3,4-dihydroquinazolin-7-yl)oxy)methyl)piperidin-1-yl)azetidin-1-yl)piperidin-1-yl)phenyl)amino)piperidine-2,6-dione formate (Example 146) using appropriate building blocks and modified reaction conditions (such as reagents, reagent ratio, temperature, and reaction time) and purification conditions as needed.

| Example | Structure | Analytical Data |
|---|---|---|
| Example 232 | 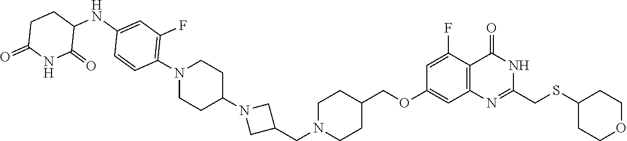 [SMILES: O=C1CCC(Nc2ccc(N3CCC(N4CC(CN5CCC(COc6cc(F)c7c(=O)[nH]c(CSC8CCOCC8)nc7c6)CC5)C4)CC3)c(F)c2)C(=O)N1] | LCMS (ESI, m/z): 780.30 [M + H]⁺. 1H NMR (400 MHz, DMSO-d6) 1H NMR (400 MHz, DMSO-d6) δ 10.80 (s, 1H), 6.89- 6.67 (m, 3H), 6.56- 6.46 (m, 1H), 6.45- 6.37 (m, 1H), 6.79 (t, |
| | 3-((3-fluoro-4-(4-(3-((4-(((5-fluoro-4-oxo-2- | J = 12.0 Hz, 1H), 4.31- |
| | (((tetrahydro-2H-pyran-4-yl)thio)methyl)-3,4- | 4.11 (m, 2H), 4.09- |
| | dihydroquinazolin-7-yl)oxy)methyl)piperidin-1- | 3.90 (m, 4H), 3.87- |
| | yl)methyl)azetidin-1-yl)piperidin-1- | 3.69 (m, 3H), 3.58- |
| | yl)phenyl)amino)piperidine-2,6-dione | 3.50 (m, 2H), 3.38- |
| | | 3.24 (m, 1H), 3.23- |
| | | 3.15 (m, 2H), 3.14- |
| | | 2.93 (m, 3H), 2.88-2.80 |
| | | (m, 1H), 2.80-2.47 |
| | | (m, 6H), 2.46-2.40 |
| | | (m, 1H), 2.35-2.23 |
| | | (m, 1H), 2.13-2.04 |
| | | (m, 2H), 2.03-1.70 |
| | | (m, 9H), 1.69-1.57 |
| | | (m, 1H), 1.56-1.29 |
| | | (m, 5H). |
| Example 233 | 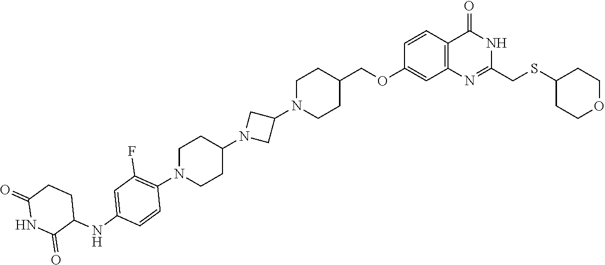 [SMILES: O=C1CCC(Nc2ccc(N3CCC(N4CC(N5CCC(COc6ccc7c(=O)[nH]c(CSC8CCOCC8)nc7c6)CC5)C4)CC3)c(F)c2)C(=O)N1] | LCMS (ESI, m/z): 748.35 [M + H]⁺. 1H NMR (400 MHz, DMSO-d6) δ 12.09 (br, 1H), 10.77 (s, 1H), 7.98 (d, J = 8.7 Hz, 1H), 7.10-7.01 (m, 2H), 6.83 (t, J = 9.3 Hz, 1H), 6.50 (dd, J = 15.0, 2.5 Hz, 1H), 6.41 (dd, J = 8.7, 2.5 Hz, 1H), 5.81-5.74 (m, 1H), 4.30-4.20 (m, 1H), 3.97 (d, J = 5.7 Hz, 2H), 3.82 (dt, J = 11.6, 3.8 Hz, 2H), 3.65 |
| | 3-((3-fluoro-4-(4-(3-(4-(((4-oxo-2-(((tetrahydro-2H- | (s, 2H), 3.42-3.34 (m, |
| | pyran-4-yl)thio)methyl)-3,4-dihydroquinazolin-7- | 3H), 3.12-3.01 (m, |
| | yl)oxy)methyl)piperidin-1-yl)azetidin-1-yl)piperidin-1- | 3H), 2.82-2.69 (m, |
| | yl)phenyl)amino)piperidine-2,6-dione | 6H), 2.60-2.50 (m, |
| | | 3H), 2.13-2.00 (m, |
| | | 2H), 1.93-1.8 (m, 3H), |
| | | 1.79-1.68 (m, 7H), |
| | | 1.53-1.4 (m, 2H), 1.39- |
| | | 1.24 (m, 5H). |
| Example 234 | 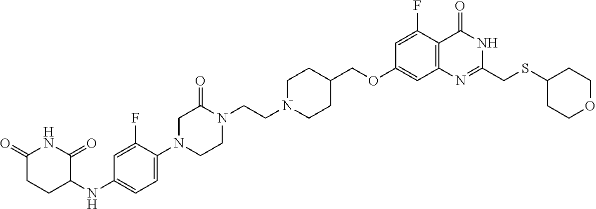 [SMILES: O=C1CCC(Nc2ccc(N3CCN(CCN4CCC(COc5cc(F)c6c(=O)[nH]c(CSC7CCOCC7)nc6c5)CC4)C(=O)C3)c(F)c2)C(=O)N1] | LCMS (ESI, m/z): 754.35 [M + H]⁺. 1H NMR (400 MHz, DMSO-d6) δ 12.16 (s, 1H), 10.78 (s, 1H), 6.92-6.80 (m, 3H), 6.55 (dd, J = 14.9, 2.5 Hz, 1H), 6.45 (dd, J = 8.7, 2.6 Hz, 1H), 5.90 (d, J = 7.8 Hz, 1H), 4.33-4.23 (m, 1H), 3.98 (d, J = 5.9 Hz, 2H), 3.82 (dt, J = 11.6, |
| | 3-((3-fluoro-4-(4-(2-(4-(((5-fluoro-4-oxo-2- | 3.8 Hz, 2H), 3.62 (s, |
| | (((tetrahydro-2H-pyran-4-yl)thio)methyl)-3,4- | 2H), 3.50-3.40 (m, |
| | dihydroquinazolin-7-yl)oxy)methyl)piperidin-1- | 5H), 3.29-3.28 (m, 3H), |
| | yl)ethyl)-3-oxopiperazin-1- | 3.20-3.10 (m, 2H), |
| | yl)phenyl)amino)piperidine-2,6-dione | 3.09-3.00 (m, 1H), 2.95 |
| | | (d, J = 10.8 Hz, 2H), |
| | | 2.80-2.69 (m, 1H), 2.62- |
| | | 2.53 (m, 1H), 2.46- |
| | | 2.41 (m, 2H), 2.13- |
| | | 1.85 (m, 6H), 1.75 (d, |
| | | J = 11.3 Hz, 3H), 1.52- |
| | | 1.38 (m, 2H), 1.32-1.20 |
| | | (m, 2H). |
| Example 235 | 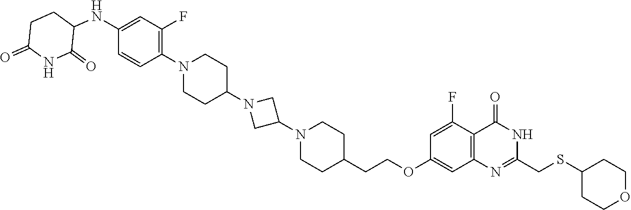 [SMILES: O=C1CCC(Nc2ccc(N3CCC(N4CC(N5CCC(CCOc6cc(F)c7c(=O)[nH]c(CSC8CCOCC8)nc7c6)CC5)C4)CC3)c(F)c2)C(=O)N1] | LCMS (ESI, m/z): 780.35 [M + H]⁺. 1H NMR (300 MHz, DMSO-d6) δ 12.15 (br, 1H), 10.75 (s, 1H), 6.92-6.77 (m, 3H), 6.56-6.37 (m, 2H), 5.76 (d, J = 7.6 Hz, 1H), 4.26 (dd, J = 11.9, 7.0 Hz, 1H), 4.15 (t, J = 6.6 Hz, 2H), 3.82 (dt, J = 11.7, 3.8 Hz, 2H), 3.62 (s, 2H), 3.41- |
| | 3-((3-fluoro-4-(4-(3-(4-(2-((5-fluoro-4-oxo-2- | 3.30 (m, 5H), 3.10-2.99 |
| | (((tetrahydro-2H-pyran-4-yl)thio)methyl)-3,4- | (m, 2H), 2.74-2.64 |
| | dihydroquinazolin-7-yl)oxy)ethyl)piperidin-1- | (m, 6H), 2.63-2.52 |
| | yl)azetidin-1-yl)piperidin-1- | (m, 2H), 2.15-2.01 |
| | yl)phenyl)amino)piperidine-2,6-dione. | (m, 2H), 1.95-1.79 |
| | | (m, 3H), 1.70-1.60 |
| | | (m, 7H), 1.55-1.36 |
| | | (m, 3H), 1.15-1.35 |
| | | (m, 6H) |
| Example 236 | 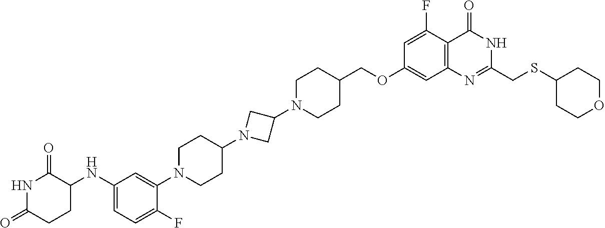 [SMILES: O=C1CCC(Nc2ccc(F)c(N3CCC(N4CC(N5CCC(COc6cc(F)c7c(=O)[nH]c(CSC8CCOCC8)nc7c6)CC5)C4)CC3)c2)C(=O)N1] | LCMS (ESI, m/z): 766.40 [M + H]⁺. 1H NMR (400 MHz, DMSO-d6) δ 10.75 (s, 1H), 6.89-6.76 (m, 3H), 6.39-6.34 (m, 1H), 6.22-6.15 (m, 1H), 5.64 (d, J = 7.5 Hz, 1H), 4.38-4.22(m, 1H), 3.97 (d, J = 5.8 Hz, 2H) 3.86-3.78 (m, 2H), 3.64 (s, 2H), 3.43-3.33(m, 3H), 3.24-3.16 (m, 2H), |
| | 3-((4-fluoro-3-(4-(3-(4-(((5-fluoro-4-oxo-2- | 3.11-3.01 (m, 1H) |
| | (((tetrahydro-2H-pyran-4-yl)thio)methyl)-3,4- | 2.82-2.79 (m, 1H), |
| | dihydroquinazolin-7-yl)oxy)methyl)piperidin-1- | 2.78-2.55 (m, 7H), |
| | yl)azetidin-1-yl)piperidin-1- | 2.14-2.06 (m, 2H), |
| | yl)phenyl)amino)piperidine-2,6-dione | 1.94-1.87 (m, 2H), |
| | | 1.79-1.69 (m, 7H), |
| | | 1.52-1.38 (m, 2H), |
| | | 1.34-1.21 (m, 7H). |
| Example 237 | 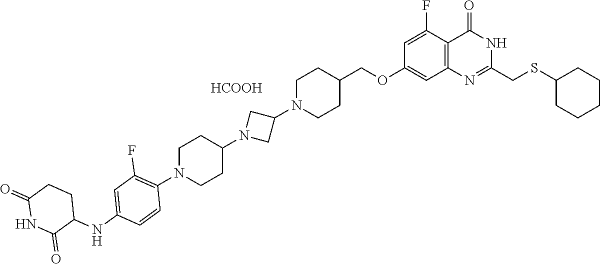 [SMILES: O=C1CCC(Nc2ccc(N3CCC(N4CC(N5CCC(COc6cc(F)c7c(=O)[nH]c(CSC8CCCCC8)nc7c6)CC5)C4)CC3)c(F)c2)C(=O)N1 ; "HCOOH"] | LCMS (ESI, m/z): 764.10 [M + H]⁺. 1H NMR (400 MHz, DMSO-d6) δ 12.15 (s, 1H), 10.78 (s, 1H), 8.26 (s, 1H), 6.92- 6.78 (m, 3H), 6.50 (m, 1H), 6.41 (d, J = 8.6 Hz, 1H), 5.79 (d, J = 7.7 Hz, 1H), 4.27-4.17 (m, 1H), 3.98 (d, J = 5.9 Hz, 2H), 3.58-3.51 (m, 3H), 3.05 (d, J = 10.6 Hz, 2H), 2.85- 2.65 (m, 7H), 2.59-2.51 (m, 2H), 2.08-2.01 (m, |
| | 3-({4-[4-(3-{4-[({2-[(cyclohexylsulfanyl)methyl]-5- | 2H), 1.95-1.81 (m, |
| | fluoro-4-oxo-3H-quinazolin-7- | 3H), 1.80-1.65 (m, 9H), |
| | yl}oxy)methyl]piperidin-1-yl}azetidin-1-yl)piperidin- | 1.55-1.47 (m, 1H), |
| | 1-yl]-3-fluorophenyl}amino)piperidine-2,6-dione | 1.31-1.15 (m, 9H). |
| | formate | |

### Example A: NanoLuc Assay for the Degradation of PARP14

### NanoLuc Plasmid

The catalytic domain of human PARP14 (residues 1611 to 1801, GenBank Accession No. NM_017554) was inserted into the pcDNA3.1(−) vector. The insert also contained a NanoLuc tag on the N terminus of the PARP14 protein.

### Assay for Degradation of PARP14

Degradation of PARP14 protein was assessed using measurement of the NanoLuc tag as proxy for the PARP14 protein. PARP14 with NanoLuc tag was overexpressed in HEK-293T cells (ATCC) using the plasmid described in Table 1. Plasmid DNA was diluted in empty vector DNA then added to 1.163 mL of phenol red free OptiMEM (Thermo Fisher). Plasmid DNA concentrations used in each assay described in Table 1. The plasmid DNA was mixed with 78.5 μL of Fugene HD (Promega) and allowed to incubate 5 minutes.

TABLE 1

| | Concentration of | Concentration of Empty |
|---|---|---|
| Plasmid | Plasmid DNA | Vector |
| PARP14 | 0.0002 μg/μl | 0.0198 μg/μl |
| (pRTX240) | | |

Next, 1.125 mL were added to 10 million 293T cells in DMEM (Thermo Fisher) supplemented with 10% FBS (VWR) and 1× Glutamax (Gibco). The transfection was incubated for 24 hrs at 37 degrees in an incubator supplemented with 5% CO2. The cells were then trypsinized and brought up in phenol red free OptiMEM media. Transfected HEK-293T cells were diluted to 125,000 cells per mL and then added to assay plate (Corning 3574) using a Multidrop (Thermo Fisher) to add 40 μL per well of the 384 well plate, resulting in 5,000 cells per well. 40 nL of a dose response curve diluted in DMSO of each test compound was added to the cell plate using a Mosquito (TTP Labtech) and the plate was incubated at 37° C. for 2 hours. Assay plate was brought to room temperature then 20 μL per well of NanoGlo (Promega) was added to the plate. Luminescence was measured on an Envision (Perkin Elmer).

The average DMSO was calculated from 32 wells containing 0.1% DMSO only in columns 12 and 24 of the assay plate. The % of DMSO values were calculated as described below:

$$\% of DMSO = \frac{Luminescence}{Avg DMSO} * 100$$

The % of DMSO values were plotted as a function of compound concentration and the following 4-parameter fit was applied to derive the DC₅₀ values:

$$Y = Bottom + \frac{( Top - Bottom )}{( 1 + {( \frac{X}{D {C}_{50}} )}^{Hill Coefficient}}$$

where top, bottom, and Hill Coefficient are allowed to float. Y is the % of DMSO and X is the compound concentration

DC₅₀ data for the Example compounds is provided below in Table 2 (“+” is <0.1 μM; “++” is ≥0.1 μM and <1 μM; and “+++” is ≥1 μM).

### Example B: HiBiT Assay for the Degradation of PARP14

### HiBiT Tagged PARP14 in LgBiT Expressing Jurkat Cells

Jurkat cells stably transfected with LgBiT (Promega) were engineered to contain a HiBiT tag on both alleles of the PARP14 gene (Genbank Accession Number: NM_017554) via CRISPR/Cas9 editing. The HiBiT tag is an 11 amino acid tag created by Promega that associates with LgBiT protein to form the NanoLuc® tag on the C-terminus of PARP14. Clones were isolated and confirmed for the HiBiT tag via Sanger sequencing.

### Assay for Degradation of PARP14

Degradation of PARP14 protein was assessed by measuring the luminescence of the HiBiT tag associating with the LgBiT protein as a proxy for the PARP14 protein.

Jurkat cells were diluted to 250,000 cells per mL and then added to assay plate (Corning 3574) using a Multidrop (Thermo Fisher) to add 20 μL per well of the 384 well plate, resulting in 5,000 cells per well. 20 nL of a dose response curve diluted in DMSO of each test compound was added to the cell plate using a Mosquito (TTP Labtech) and the plate was incubated at 37° C. for 2 or 24 hours. Assay plate was brought to room temperature then 5 μL per well of Live Cell Substrate (Promega) was added to the plate. Luminescence was measured on an Envision (Perkin Elmer).

The average DMSO was calculated from 32 wells containing 0.1% DMSO only in columns 12 and 24 of the assay plate. The % of DMSO values were calculated as described below:

$$\% of DMSO = \frac{Luminescence}{Avg DMSO} * 100$$

The % of DMSO values were plotted as a function of compound concentration and the following 4-parameter fit was applied to derive the DC₅₀ values:

$$Y = Bottom + \frac{( Top - Bottom )}{( 1 + {( \frac{X}{D {C}_{50}} )}^{Hill Coefficient}}$$

where top, bottom, and Hill Coefficient are allowed to float. Y is the % of DMSO and X is the compound concentration.

DC₅₀ data for the Example compounds is provided below in Table 2 (“+” is ≤0.03 μM; “++” is ≥0.03 μM).

TABLE 2
DC₅₀ Data for Example Compounds

| Example | | |
|---|---|---|
| No. | DC₅₀ | Assay¹ |
| 1 | + | A |
| 2 | + | A |
| 3 | + | A |
| 4 | + | A |
| 5 | + | A |
| 6 | + | A |
| 7 | + | A |
| 8 | + | A |
| 9 | + | A |
| 10 | + | A |
| 11 | + | A |
| 12 | + | A |
| 13 | + | A |
| 14 | + | A |
| 15 | + | A |
| 16 | + | A |
| 17 | + | A |
| 18 | + | A |
| 19 | + | A |
| 20 | + | A |
| 21 | + | A |
| 22 | + | A |
| 23 | + | A |
| 24 | + | A |
| 25 | + | A |
| 26 | + | A |
| 27 | + | A |
| 28 | + | A |
| 29 | + | A |
| 30 | + | A |
| 31 | + | A |
| 32 | + | A |
| 33 | + | A |
| 34 | + | A |
| 35 | + | A |
| 36 | + | A |
| 37 | + | A |
| 38 | + | A |
| 39 | + | A |
| 40 | + | A |
| 41 | + | A |
| 42 | + | A |
| 43 | + | A |
| 44 | + | A |
| 45 | + | A |
| 46 | + | A |
| 47 | + | A |
| 48 | + | A |
| 48a | + | A |
| 48b | + | A |
| 49 | + | A |
| 50 | + | A |
| 51 | + | A |
| 52 | + | A |
| 53 | + | A |
| 54 | + | A |
| 55 | + | A |
| 56 | + | A |
| 57a | + | A |
| 57b | + | A |
| 58 | + | A |
| 59 | + | A |
| 60 | + | A |
| 61 | + | A |
| 62 | + | A |
| 63 | + | A |
| 64 | + | A |
| 64a | + | B |
| 64b | + | B |
| 65 | + | A |
| 66 | + | A |
| 67 | + | A |
| 68 | + | A |
| 69 | + | A |
| 70 | + | A |
| 71 | + | A |
| 72 | + | A |
| 73 | + | A |
| 74 | + | A |
| 75 | + | A |
| 76 | + | A |
| 77 | + | A |
| 78 | + | A |
| 79a | + | A |
| 79b | + | A |
| 80a | + | A |
| 80b | + | A |
| 81 | + | A |
| 82 | + | A |
| 83 | + | A |
| 84 | + | A |
| 85 | + | A |
| 86 | + | A |
| 87 | + | A |
| 88 | + | A |
| 89 | + | A |
| 90 | + | A |
| 91 | + | B |
| 92 | + | B |
| 93 | + | B |
| 94 | + | A |
| 95 | + | A |
| 96 | + | A |
| 97 | + | B |
| 98 | + | B |
| 99 | + | B |
| 99a | + | B |
| 99b | + | B |
| 100 | + | B |
| 101 | + | B |
| 102 | + | B |
| 103 | + | B |
| 104 | + | B |
| 105 | + | B |
| 106 | + | B |
| 107 | + | B |
| 108 | + | B |
| 109 | + | A |
| 110 | + | B |
| 111 | + | B |
| 112 | + | B |
| 113 | + | B |
| 114 | + | B |
| 115 | + | B |
| 116 | + | B |
| 116a | + | B |
| 116b | + | B |
| 117 | + | B |
| 117a | + | B |
| 117b | + | B |
| 118 | + | B |
| 119 | + | B |
| 120 | + | B |
| 121 | + | A |
| 122 | + | A |
| 123 | + | A |
| 124 | + | A |
| 125 | + | B |
| 126 | + | B |
| 127 | + | B |
| 128 | + | B |
| 129 | + | B |
| 130 | + | B |
| 131 | + | B |
| 132 | + | B |
| 133 | + | B |
| 134 | + | B |
| 135 | + | B |
| 136 | + | B |
| 136a | + | B |
| 136b | + | B |
| 137 | + | B |
| 138 | + | B |
| 139 | + | B |
| 140 | + | B |
| 141 | + | B |
| 142 | + | B |
| 143 | + | B |
| 144 | + | B |
| 145 | + | B |
| 146 | + | B |
| 147 | + | B |
| 148 | + | B |
| 149 | + | B |
| 150 | + | B |
| 151 | + | B |
| 152 | + | B |
| 153 | + | B |
| 154 | + | B |
| 155 | + | B |
| 156 | + | B |
| 157 | + | B |
| 158 | + | B |
| 159 | + | B |
| 160 | + | B |
| 161 | + | B |
| 162 | + | B |
| 163 | + | B |
| 164 | + | B |
| 165 | + | B |
| 166 | + | B |
| 167 | + | B |
| 168 | + | B |
| 169 | + | B |
| 170 | + | B |
| 171 | + | B |
| 172 | + | B |
| 173 | + | B |
| 174 | + | B |
| 175 | + | B |
| 176 | + | B |
| 177 | + | B |
| 178 | + | B |
| 179 | + | B |
| 180 | + | B |
| 181 | + | B |
| 182 | + | B |
| 183 | + | B |
| 184 | + | B |
| 185 | + | B |
| 186 | + | B |
| 187 | + | B |
| 188 | + | B |
| 189 | + | B |
| 190 | + | B |
| 191 | + | B |
| 192 | + | B |
| 193 | + | B |
| 194 | + | B |
| 195 | + | B |
| 196 | + | B |
| 197 | + | B |
| 198 | + | B |
| 199 | + | B |
| 200 | + | B |
| 201 | + | B |
| 202 | + | B |
| 203 | + | B |
| 204 | + | B |
| 205 | + | B |
| 206 | + | B |
| 207 | + | B |
| 208 | + | B |
| 209 | + | B |
| 210 | + | B |
| 211 | + | B |
| 212 | + | B |
| 213 | + | B |
| 214 | + | B |
| 215 | + | B |
| 216 | + | B |
| 217 | + | B |
| 218 | + | B |
| 219 | + | B |
| 220 | + | B |
| 221 | + | B |
| 222 | + | B |
| 223 | + | B |
| 224 | + | B |
| 225 | + | B |
| 226 | + | B |
| 227 | + | B |
| 228 | + | B |
| 229 | + | B |
| 230 | + | B |
| 231 | + | B |
| 232 | + | B |
| 233 | + | B |
| 234 | + | B |
| 235 | + | B |
| 236 | + | B |
| 237 | + | B |

¹“A” designates assay described in Example A; “B” designates assay described in Example B

### Example C: Decrease of Cell Counts and Cytokines in BALF and Lung Homogenate Following Alternaria-Sensitization and Treatment with a PARP14 Degrader

The effect of Compound 64 was studied in an Alternaria asthma mouse model. On days 1-5, male Balb/c mice under isoflurane anesthesia were challenged by instilling a solution of 5 μg (protein weight) Alternaria in 40 μL of PBS into each nostril. Sham-treated mice were administered 40 μL of PBS into each nostril. Compound 64 was administered two days prior to Alternaria challenge (defined day as day −1) and animals were treated with vehicle (0.5% methylcellulose+0.2% Tween 20) or Compound Y (30, 100, 300, 1000 mg/kg) once daily for 7 days (defined as day −1 to day 5) by oral gavage. Total and differential cell counts of the BALF fluid samples were measured using a XT-2000iV analyzer (Sysmex). Cytokine concentrations of BALF supernatant were measured in samples from all groups using ELISA kit (Biotechne, UK).

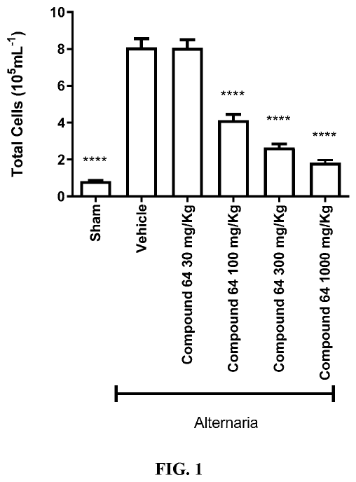

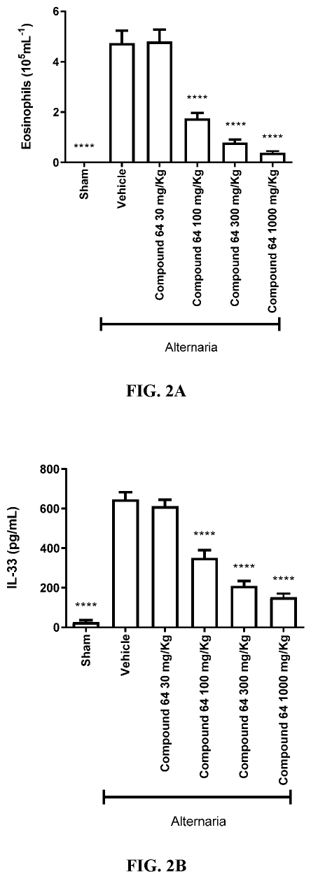

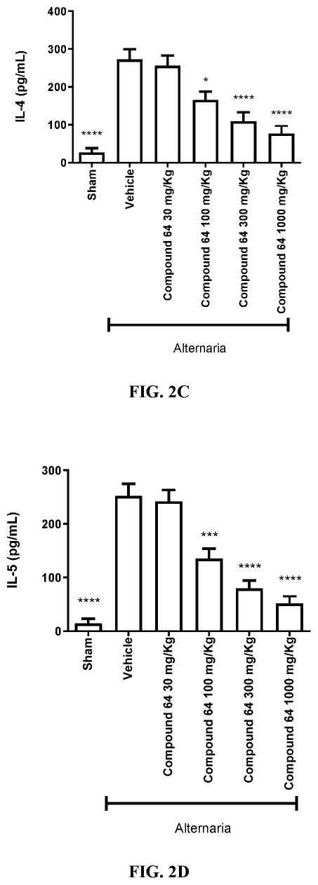

Statistical significance is calculated using one-way ANOVA followed by Dunnett's post-tests in which the treatment groups were compared to vehicle control (P<0.05). FIG. 1 illustrates that Compound 64 significantly reduces total cell counts in a dose-dependent manner starting from 100 mg/kg dose. FIG. 2 illustrates that Compound 64 also significantly reduces eosinophil and cytokines IL-33, IL-4, and IL-5 in BALF in a dose-dependent manner starting from 100 mg/kg.

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

What is claimed is:

1. A compound of Formula (I): 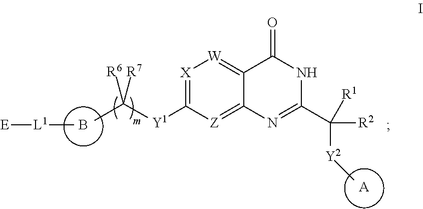
or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, —O—, and —(C₂₋₄ alkynyl)-;
Y² is selected from bond, —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and —(NR³)CH₂—;
Ring A is selected from 6-10 membered aryl, 5-10 membered heteroaryl, C₃₋₁₄ cycloalkyl, and 4-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C_{2-M} dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{cl}R^{d1}, NR^{c1}C(O)R^{b1}, NR^{c1}C(O)OR^{a1} NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{a1}, NR^{c1}C(═NR^{e1})NR^{c1}R^{d1}, NR^{c1}R^{d1}, NR^{c1}C(O)R^{b1}, NR^{c1}C(O)OR^{a1} NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl,
C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR²C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3} NR³C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R⁴, NR^{c4}S(O)₂NR^{c4}R^{d4}, and S(O)₂NR^{c4}R^{d4};
R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁-4 alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{e2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase; and
L¹ is a linker, which is covalently linked to moiety Q and to moiety E;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

2. A compound of Formula (I): 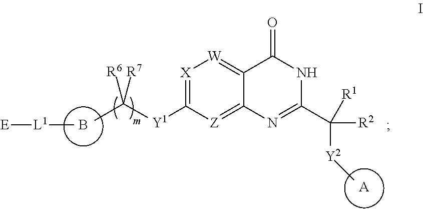
or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, and —O—;
Y² is selected from bond, —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and —(NR³)CH₂—;
Ring A is selected from 6-10 membered aryl, 5-10 membered heteroaryl, C₃₋₁₄ cycloalkyl, and 4-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋ₛ dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋ₛ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{d1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1} NR^{c1}C(O)NR^{c1}R^{d1}, C(═NR^{e1})R^{b1}, C(═NR^{e1})NR^{c1}R^{d1}, NR^{c1}C(═—NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR²C(O)R^{b2} NR^{c2}C(O)OR^{a2}, NR²C(O)NR^{c2}R^{d2}, C(═NR^{e2})R^{b2}, C(═NR^{e2})NR^{c2}R^{a2}, NR^{c2}C(═NR^{e2})NR^{c2}R^{d2}, NR^{c2}S(O)R^{b2}NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl,
C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR²C(═NR^{e2})NR^{c2}R^{d2}, NR^{c2}R^{d2}, NR^{c2}C(O)R^{b2}, NR^{c2}C(O)OR^{a2} NR^{c2}C(O)NR^{c2}R^{d2}, NR²S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3} NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3} NR^{c3}C(O)R^{b3}, NR³C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b1}, R^{c1}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{e2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase; and
L¹ is a linker, which is covalently linked to moiety Q and to moiety E;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

3. A compound of Formula (I): 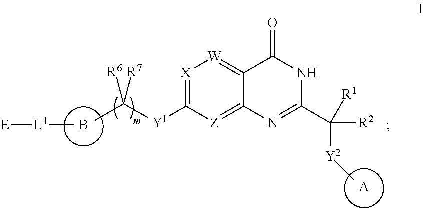
or a pharmaceutically acceptable salt thereof, wherein:
W is CR^{W} or N;
X is CR^{X} or N;
Z is CR^{Z} or N; and
wherein no more than two of W, X, and Z are simultaneously N;
Y¹ is selected from —NR³—, —CR⁴R⁵—, and —O—;
Y² is selected from —S—, —S(O)—, —S(O)₂—, —CH₂—, —O—, —N(R³)—, —SCH₂—, —S(O)CH₂—, —S(O)₂CH₂—, —CH₂CH₂—, —OCH₂—, and —(NR³)CH₂—;
Ring A is selected from 6-10 membered aryl, 5-10 membered heteroaryl, C₃₋₁₄ cycloalkyl, and 4-18 membered heterocycloalkyl, wherein Ring A is optionally substituted by 1, 2, 3, or 4 R^{A};
Ring B is selected from 6-10 membered aryl, 5-10 membered heteroaryl, C₃₋₁₄ cycloalkyl, and 4-18 membered heterocycloalkyl, wherein Ring B is optionally substituted by 1, 2, 3, or 4 R^{B};
R¹ and R² are each, independently, selected from H and methyl;
R³ is selected from H and C₁₋₄ alkyl;
R⁴ and R⁵ are each, independently, selected from H, C₁₋₄ alkyl, C₁₋₄ alkoxy, C₁₋₄ haloalkyl, amino, C₁₋₄ alkylamino, and C₂₋₈dialkylamino;
R⁶ and R⁷ are each, independently, selected from H, halo, C₁₋₄ alkyl, C₁₋₄ alkoxy, C₁₋₄ haloalkyl, amino, C₁₋₄ alkylamino, and C₂₋₈ dialkylamino;
each R^{A} is independently selected from halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}C(O)OR^{a1} NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1} NRCS(O)R^{b1}, NR^{c1}S(O)₂R^{b1}, NR^{c1}S(O)₂NR^{c1}R^{d1}, S(O)R^{b1}, S(O)NR^{cl}R^{d1}, S(O)₂R^{b1}, and S(O)₂NR^{c1}R^{d1}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{A} are each optionally substituted with 1, 2, 3, 4, or 5 substituents independently selected from Cy¹, Cy¹-C₁₋₄ alkyl, halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{cl}R^{d1}, NR^{c1}R^{d1} NR^{c1}C(O)R^{b1}, NR^{c1}C(O)OR^{a1} NR^{c1}C(O)NR^{c1}R^{d1} NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each R^{B} is independently selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR²C(O)R^{b2} NR^{c2}C(O)OR^{a2}, NR²C(O)NR^{c2}R^{d2}, C(═NR^{e2})R^{b2}, C(═NR^{e2})NR^{c2}R^{d2}, NR^{c2}C(═NR^{e2})NR^{c2}R^{d2}, NR^{c2}S(O)R^{b2}NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R²; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl,
C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{B} is optionally substituted with 1, 2, 3, 4, or 5 substituents independently selected from Cy², Cy²-C₁₋₄ alkyl, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR²C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2}, NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
R^{W}, R^{X}, and R^{Z} are each, independently, selected from H, halo, C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}; wherein said C₁₋₆ alkyl, C₂₋₆ alkenyl, C₂₋₆ alkynyl, C₁₋₆ haloalkyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl,
C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{W}, R^{X}, or R^{Z} are each optionally substituted with 1, 2, 3, 4, or 5 substituents independently selected from Cy³, Cy³-C₁₋₄ alkyl, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3};
each Cy¹ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, C(═NR^{e1})NR^{c1}R^{d1}, NR^{c1}C(═NR^{e1})NR^{c1}R^{d1}, NR^{c1}R^{d1}, NR^{c1}C(O)R^{b1}, NR^{c1}C(O)OR^{a1}, NR^{c1}C(O)NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1};
each Cy² is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, C(═NR^{e2})NR^{c2}R^{d2}, NR^{c2}C(═NR^{e2})NR^{c2}R^{d2}, NR^{c2}R^{d2}, NR^{c2}C(O)R^{b2}, NR^{c2}C(O)OR^{a2}, NR^{c2}C(O)NR^{c2}R^{d2}, NR^{c2}S(O)R^{b2}, NR^{c2}S(O)₂R^{b2} NR^{c2}S(O)₂NR^{c2}R^{d2}, S(O)R^{b2}, S(O)NR^{c2}R^{d2}, S(O)₂R^{b2}, and S(O)₂NR^{c2}R^{d2};
each Cy³ is independently selected from C₆₋₁₀ aryl, C₃₋₇ cycloalkyl, 5-10 membered heteroaryl, and 4-10 membered heterocycloalkyl, each optionally substituted by 1, 2, 3, or 4 substituents independently selected from halo, C₁₋₆ alkyl, C₂₋₆ alkenyl, C₂₋₆ alkynyl, C₁₋₆ haloalkyl, C₆₋₁₀ aryl-C₁₋₄ alkyl, C₃₋₇ cycloalkyl-C₁₋₄ alkyl, 5-10 membered heteroaryl-C₁₋₄ alkyl, 4-10 membered heterocycloalkyl-C₁₋₄ alkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, C(═NR^{e3})NR^{c3}R^{d3}, NR^{c3}C(═NR^{e3})NR^{c3}R^{d3}, NR^{c3}R^{d3} NR^{c3}C(O)R^{b3}, NR³C(O)OR^{a3}, NR^{c3}C(O)NR^{c3}R^{d3}, NR^{c3}S(O)R^{b3}, NR^{c3}S(O)₂R^{b3}, NR^{c3}S(O)₂NR^{c3}R^{d3} S(O)R^{b3}, S(O)NR^{c3}R^{d3}, S(O)₂R^{b3}, and S(O)₂NR^{c3}R^{d3};
each R^{a1}, R^{b1}, R^{c1}, R^{d1}, R^{a2}, R^{b2}, R^{c2}, R^{d2}, R^{a3}, R^{b3}, R^{c3}, and R^{d3} is independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl of R^{a1}, R^{b}, R^{c}, R^{d1}, R^{a2}, R^{b2}, R^{c2}, R^{d2}, R^{a3}, R^{b3}, R^{c3}, or R^{d3} is optionally substituted with 1, 2, 3, 4, or 5 substituents independently selected from Cy⁴, Cy⁴-C₁₋₄ alkyl, halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR⁴C(═NR^{e4})NR^{c4}R^{d4}, S(O)R^{b4}, S(O)NR^{c4}R^{d4}, S(O)₂R^{b4}, NR^{c4}S(O)₂R^{b4}, NR^{c4}S(O)₂NR^{c4}R^{d4}, and S(O)₂NR^{c4}R^{d4};
each Cy⁴ is C₆₋₁₀ aryl, C₃₋₇ cycloalkyl, 5-10 membered heteroaryl, or 4-10 membered heterocycloalkyl, each optionally substituted by 1, 2, 3, or 4 substituents independently selected from halo, C₁₋₄ alkyl, C₁₋₄ haloalkyl, C₁₋₆ haloalkyl, C₂₋₆ alkenyl, C₂₋₆ alkynyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4}, NR^{c4}C(O)R^{b4}, NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4}, S(O)R^{b4}, S(O)NR^{c4}R^{d4}, S(O)₂R^{b4}, NR^{c4}S(O)₂R⁴, NR^{c4}S(O)₂NR^{c4}R^{d4}, and S(O)₂NR^{c4}R^{d4};
R^{a4}, R^{b4}, R^{c4}, and R^{d4} are independently selected from H, C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl, wherein said C₁₋₆ alkyl, C₁₋₆ haloalkyl, C₂₋₆ alkenyl, C₂₋₆ alkynyl, C₆₋₁₀ aryl, C₃₋₇ cycloalkyl, 5-10 membered heteroaryl, 4-10 membered heterocycloalkyl, C₆₋₁₀ aryl-C₁₋₄ alkyl, C₃₋₇ cycloalkyl-C₁₋₄ alkyl, 5-10 membered heteroaryl-C₁₋₄ alkyl, and 4-10 membered heterocycloalkyl-C₁₋₄ alkyl are each optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
or R^{c1} and R^{d1} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{e2} and R^{d2} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
or R^{c3} and R^{d3} together with the N atom to which they are attached form a 4-7 membered heterocycloalkyl group optionally substituted with 1, 2, or 3 substituents independently selected from halo, C₁₋₄ alkyl, C₁₋₄ haloalkyl, CN, OR^{a4}, SR^{a4}, C(O)R^{b4}, C(O)NR^{c4}R^{d4}, C(O)OR^{a4}, OC(O)R^{b4}, OC(O)NR^{c4}R^{d4}, NR^{c4}R^{d4} NR^{c4}C(O)R^{b4} NR^{c4}C(O)NR^{c4}R^{d4}, NR^{c4}C(O)OR^{a4}, C(═NR^{e4})NR^{c4}R^{d4}, NR^{c4}C(═NR^{e4})NR^{c4}R^{d4} S(O)R^{b4}, S(O)NR^{c4}R^{d4}, S(O)₂R^{b4}, NR^{c4}S(O)₂R^{b4}, NR^{c4}S(O)₂NR^{c4}R^{d4} and S(O)₂NR^{c4}R^{d4};
each R^{e1}, R^{e2}, R^{e3}, and R^{e4} is independently selected from H, C₁₋₄ alkyl, and CN;
m is 0, 1, or 2;
E is an E3 ubiquitin ligase binding moiety, which binds to an E3 ubiquitin ligase; and
L¹ is a linker, which is covalently linked to moiety Q and to moiety E;
wherein any aforementioned heteroaryl or heterocycloalkyl group comprises 1, 2, 3, or 4 ring-forming heteroatoms independently selected from O, N, and S;
wherein one or more ring-forming C or N atoms of any aforementioned heterocycloalkyl group is optionally substituted by an oxo (═O) group; and
wherein one or more ring-forming S atoms of any aforementioned heterocycloalkyl group is optionally substituted by one or two oxo (═O) groups.

4. The compound of claim 1, or a pharmaceutically acceptable salt thereof, wherein W is CR^{W}.

5. The compound of claim 1, or a pharmaceutically acceptable salt thereof, wherein X is CR^{X}.

6. The compound of claim 1, or a pharmaceutically acceptable salt thereof, wherein Z is CR^{Z}.

7. The compound of claim 1, or a pharmaceutically acceptable salt thereof, wherein Y¹ is —O—.

8. The compound of claim 1, or a pharmaceutically acceptable salt thereof, wherein Y¹ is —CR⁴R⁵—.

9. The compound of claim 1, or a pharmaceutically acceptable salt thereof, wherein Y¹ is —NR³—.

10. The compound of claim 1, or a pharmaceutically acceptable salt thereof, wherein Y¹ is —(C₂₋₄ alkynyl)-.

11. The compound of claim 1, or a pharmaceutically acceptable salt thereof, wherein Y² is —S—.

12. The compound of claim 1, or a pharmaceutically acceptable salt thereof, wherein Y² is —CH₂—.

13. The compound of claim 1, or a pharmaceutically acceptable salt thereof, wherein Y² is —O—.

14. The compound of claim 1, or a pharmaceutically acceptable salt thereof, wherein Y² is bond.

15. The compound of claim 1, or a pharmaceutically acceptable salt thereof, wherein Ring A is 4-18 membered heterocycloalkyl, wherein Ring A is optionally substituted by 1, 2, 3, or 4 R^{A}.

16. The compound of claim 1, or a pharmaceutically acceptable salt thereof, wherein Ring A is 4-7 membered heterocycloalkyl, wherein Ring A is optionally substituted by 1, 2, 3, or 4 R^{A}.

17. The compound of claim 1, or a pharmaceutically acceptable salt thereof, wherein Ring A is tetrahydro-2H-pyranyl or piperidinyl, each optionally substituted by 1, 2, 3, or 4 R^{A}.

18. The compound of claim 1, or a pharmaceutically acceptable salt thereof, wherein Ring A is tetrahydro-2H-pyran-4-yl or 1-methylpiperidin-4-yl.

19. The compound of claim 1, or a pharmaceutically acceptable salt thereof, wherein Ring A is tetrahydro-2H-pyran-4-yl, 1-methylpiperidin-4-yl, or 1-(2,2,2-trifluoroethyl)piperidin-4-yl.

20. The compound of claim 1, or a pharmaceutically acceptable salt thereof, wherein Ring A is C₃₋₁₄ cycloalkyl, and wherein Ring A is optionally substituted by 1, 2, 3, or 4 R^{A}.

21. The compound of claim 1, or a pharmaceutically acceptable salt thereof, wherein Ring A is tetrahydro-2H-pyran-4-yl, 1-methylpiperidin-4-yl, 1-(2,2,2-trifluoroethyl)piperidin-4-yl, piperazinyl, cyclopropyl, cyclobutyl, cyclopentyl, cyclohexyl, or 4-hydroxycyclohexyl.

22. The compound of claim 1, or a pharmaceutically acceptable salt thereof, wherein Ring B is C₃₋₇ cycloalkyl or 4-7 membered heterocycloalkyl, wherein Ring B is optionally substituted by 1, 2, 3, or 4 R^{B}.

23. The compound of claim 1, or a pharmaceutically acceptable salt thereof, wherein Ring B is 4-7 membered heterocycloalkyl, wherein Ring B is optionally substituted by 1, 2, 3, or 4 R^{B}.

24. The compound of claim 1, or a pharmaceutically acceptable salt thereof, wherein Ring B is piperidinyl optionally substituted by 1, 2, 3, or 4 R^{B}.

25. The compound of claim 1, or a pharmaceutically acceptable salt thereof, wherein Ring B is piperidinyl.

26. The compound of claim 1, or a pharmaceutically acceptable salt thereof, wherein Ring B is piperidinyl, piperazinyl, or azetidinyl, each of which is optionally substituted by 1, 2, 3, or 4R^{B}.

27. The compound of claim 1, or a pharmaceutically acceptable salt thereof, wherein Ring B is C₃₋₇ cycloalkyl, and wherein Ring B is optionally substituted by 1, 2, 3, or 4 R^{B}.

28. The compound of claim 1, or a pharmaceutically acceptable salt thereof, wherein Ring B is cyclopropyl, cyclobutyl or cyclohexyl.

29. The compound of claim 1, or a pharmaceutically acceptable salt thereof, wherein R¹ and R² are each H.

30. The compound of claim 1, or a pharmaceutically acceptable salt thereof, wherein R⁴ and R⁵ are each H.

31. The compound of claim 1, or a pharmaceutically acceptable salt thereof, wherein R⁶ and R⁷ are each H.

32. The compound of claim 1, or a pharmaceutically acceptable salt thereof, wherein each R^{A} is independently selected from halo, C₁₋₆ alkyl, C₂₋₆ alkenyl, C₂₋₆ alkynyl, C₁₋₆ haloalkyl, CN, NO₂, OR^{a1}, SR^{a1}, C(O)R^{b1}, C(O)NR^{c1}R^{d1}, C(O)OR^{a1}, OC(O)R^{b1}, OC(O)NR^{c1}R^{d1}, NR^{c1}R^{d1}, NR^{c1}C(O)R^{b1}, NR^{c1}(O)OR^{a1}, NR^{c1}C(O)NR^{c1}R^{d1}, C(═NR^{e1})R^{b1}, C(═NR^{e1})NR^{c1}R^{d1}, NR^{c1}C(═NR^{e1})NR^{c1}R^{d1}, NR^{c1}S(O)R^{b1}, NR^{c1}S(O)₂R^{b1}, NR^{c1}S(O)₂NR^{c1}R^{d1}, S(O)R^{b1}, S(O)NR^{c1}R^{d1}, S(O)₂R^{b1}, and S(O)₂NR^{c1}R^{d1}.

33. The compound of claim 1, or a pharmaceutically acceptable salt thereof, wherein each R^{A} is independently selected from halo, C₁₋₆ alkyl, C₁₋₆ haloalkyl, CN, NO₂, or OR^{a1}.

34. The compound of claim 1, or a pharmaceutically acceptable salt thereof, wherein each R^{A} is C₁₋₆ alkyl.

35. The compound of claim 1, or a pharmaceutically acceptable salt thereof, wherein R^{A} is methyl.

36. The compound of claim 1, or a pharmaceutically acceptable salt thereof, wherein R^{A} is methyl, OH, or 2,2,2-trifluoroethyl.

37. The compound of claim 1, or a pharmaceutically acceptable salt thereof, wherein each R^{B} is independently selected from H, halo, C₁₋₆ alkyl, C₂₋₆ alkenyl, C₂₋₆ alkynyl, C₁₋₆ haloalkyl, CN, NO₂, OR^{a2}, SR^{a2}, C(O)R^{b2}, C(O)NR^{c2}R^{d2}, C(O)OR^{a2}, OC(O)R^{b2}, OC(O)NR^{c2}R^{d2}, NR^{c2}R^{d2}, NR^{c2}C(O)R^{b2} NR²C(O)OR^{a2}, NR^{c2}C(O)NR^{c2}R^{d2}, C(═NR^{e2})R^{b2}, C(═NR^{e2})NR^{c2}R^{d2}, NR²C(═NR^{e2})NR^{c2}R^{d2} NR^{e2}S(O)R^{b2}, NR^{c2}S(O)₂R^{b2}, NR^{c2}S(O)₂NR^{c2}R^{d2}, S(O)R^{b2}, S(O)NR^{c2}R^{d2} S(O)₂R^{b2}, and S(O)₂NR^{c2}R^{d2}.

38. The compound of claim 1, or a pharmaceutically acceptable salt thereof, wherein each R^{B} is independently selected from halo, C₁₋₆ alkyl, C₁₋₆ haloalkyl, CN, NO₂, or OR^{a2}.

39. The compound of claim 1, or a pharmaceutically acceptable salt thereof, wherein each R^{B} is independently selected from halo.

40. The compound of claim 1, or a pharmaceutically acceptable salt thereof, wherein each R^{B} is F.

41. The compound of claim 1, or a pharmaceutically acceptable salt thereof, wherein R^{W} is selected from H, halo, C₁₋₆ alkyl, C₂₋₆ alkenyl, C₂₋₆ alkynyl, C₁₋₆ haloalkyl, CN, NO₂, OR^{a3}, SR^{a3}, C(O)R^{b3}, C(O)NR^{c3}R^{d3}, C(O)OR^{a3}, OC(O)R^{b3}, OC(O)NR^{c3}R^{d3}, NR^{c3}R^{d3}, NR^{c3}C(O)R^{b3}, NR^{c3}C(O)OR^{a3}, NR^{c3}C(O)NR^{c3}R^{d3}, C(═NR^{e3})R^{b3}, C(═NR^{e3})NR^{c3}R^{d3}, NR^{c3}C(═NR^{e3})NR^{c3}R^{d3}, NR^{c3}S(O)R^{b3}, NR^{c3}S(O)₂R^{b3}, NR^{c3}S(O)₂NR^{c3}R^{d3}, S(O)R^{b3}, S(O)NR^{c3}R^{d3}, S(O)₂R^{b3}, and S(O)₂NR^{c3}R^{d3}.

42. The compound of claim 1, or a pharmaceutically acceptable salt thereof, wherein R^{W} is selected from H, halo, C₁₋₆ alkyl, C₂₋₆ alkenyl, C₂₋₆ alkynyl, C₁₋₆ haloalkyl, CN, NO₂, and OR^{a3}.

43. The compound of claim 1, or a pharmaceutically acceptable salt thereof, wherein R^{W} is selected from H, halo, and C₁₋₆ haloalkyl.

44. The compound of claim 1, or a pharmaceutically acceptable salt thereof, wherein R^{W} is F.

45. The compound of claim 1, or a pharmaceutically acceptable salt thereof, wherein R^{X} is selected from C₆₋₁₀ aryl and 5-10 membered heteroaryl, wherein said C₆₋₁₀ aryl and 5-10 membered heteroaryl are each optionally substituted with 1, 2, 3, 4, or 5 substituents independently selected from halo, C₁₋₆ alkyl, C₂₋₆ alkenyl, C₂₋₆ alkynyl, C₁₋₆ haloalkyl, CN, OR^{a3}, and SR^{a3}.

46. The compound of claim 1, or a pharmaceutically acceptable salt thereof, wherein R^{X} is selected from H, halo, C₁₋₆ alkyl, C₂₋₆ alkenyl, C₂₋₆ alkynyl, C₁₋₆ haloalkyl, CN, OR^{a3}, and C₆₋₁₀ aryl.

47. The compound of claim 1, or a pharmaceutically acceptable salt thereof, wherein R^{X} is H.

48. The compound of claim 1, or a pharmaceutically acceptable salt thereof, wherein R^{X} is H or F.

49. The compound of claim 1, or a pharmaceutically acceptable salt thereof, wherein R^{Z} is selected from H, halo, C₁₋₆ alkyl, C₂₋₆ alkenyl, C₂₋₆ alkynyl, C₁₋₆ haloalkyl, CN, NO₂, and OR^{a3}.

50. The compound of claim 1, or a pharmaceutically acceptable salt thereof, wherein R^{Z} is H.

51. The compound of claim 1, or a pharmaceutically acceptable salt thereof, wherein m is 2.

52. The compound of claim 1, or a pharmaceutically acceptable salt thereof, wherein m is 1.

53. The compound of claim 1, or a pharmaceutically acceptable salt thereof, wherein m is 0.

54. The compound of claim 1, or a pharmaceutically acceptable salt thereof, wherein L¹ is selected from the following:
(i) bond, such that ring B is directly attached to moiety E;
(ii) —(C₁₋₄ alkyl)-;
(iii) —(C₂₋₄ alkenyl)-;
(iv) —(C₂₋₄ alkynyl)-;
(V) —(C₂₋₄ alkynyl)-(G³)-;
(vi) the following structure: 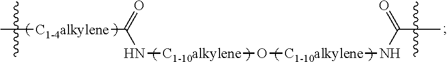
(vii) the following structure: 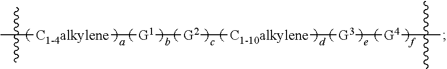
and
(viii) the following structure: 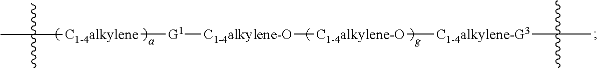
wherein G¹ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G² is C₆₋₁₀ aryl, C₃₋₇ cycloalkyl, 5-10 membered heteroaryl, or 4-10 membered heterocycloalkyl;
G³ is selected from —C(O)—, —NR^{G}C(O)—, —NR^{G}—, —O—, —S—, —C(O)O—, —OC(O)NR^{G}—, —NR^{G}C(O)NR^{G}—, —S(O₂)—, and —S(O)NR^{G}—;
G⁴ is 4-10 membered heterocycloalkyl, which is optionally substituted with C₁₋₆ alkyl, hydroxyl, C₁₋₆ alkoxy, or halo;
each R^{G} is independently selected from H, methyl, and ethyl;
a is 0 or 1;
b is 0 or 1;
c is 0 or 1;
d is 0 or 1;
e is 0 or 1;
f is 0 or 1;
g is 0, 1, or 2;
wherein at least one of b, c, e, and f is 1.

55. The compound of claim 54, or a pharmaceutically acceptable salt thereof, wherein L¹ is a bond, such that ring B is directly attached to moiety E.

56. The compound of claim 54, or a pharmaceutically acceptable salt thereof, wherein L¹ is —(C₁₋₄ alkyl)-.

57. The compound of claim 54, or a pharmaceutically acceptable salt thereof, wherein L¹ is —(C₂₋₄ alkenyl)-.

58. The compound of claim 54, or a pharmaceutically acceptable salt thereof, wherein L¹ is —(C₂₋₄ alkynyl)-.

59. The compound of claim 54, or a pharmaceutically acceptable salt thereof, wherein L¹ is —(C₂₋₄ alkynyl)-(G³)-.

60. The compound of claim 54, or a pharmaceutically acceptable salt thereof, wherein L¹ has the following structure:

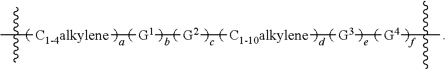

61. The compound of claim 54, or a pharmaceutically acceptable salt thereof, wherein L¹ is

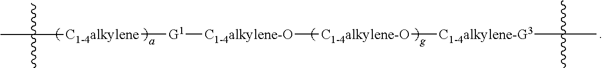

62. The compound of claim 54, or a pharmaceutically acceptable salt thereof, wherein G¹ is —NR^{G}C(O) or —C(O)—.

63. The compound of claim 54, or a pharmaceutically acceptable salt thereof, wherein G¹ is —NR^{G}C(O)—, —C(O)—, or —O—.

64. The compound of claim 54, or a pharmaceutically acceptable salt thereof, wherein G² is 4-10 membered heterocycloalkyl.

65. The compound of claim 54, or a pharmaceutically acceptable salt thereof, wherein G² is piperidinyl, piperazinyl, or azetidinyl.

66. The compound of claim 54, or a pharmaceutically acceptable salt thereof, wherein G² is azetidinyl.

67. The compound of claim 54, or a pharmaceutically acceptable salt thereof, wherein G² is piperidinyl, piperazinyl, azetidinyl, 3,6-diazabicyclo[3.1.1]heptanyl, 3,4-dihydroquinazolin-7-yl, or cyclobutyl.

68. The compound of claim 54, or a pharmaceutically acceptable salt thereof, wherein G² is piperidinyl, piperazinyl, azetidinyl, pyridinyl, 3,6-diazabicyclo[3.1.1]heptanyl, 3,4-dihydroquinazolin-7-yl, cyclobutyl, 3,8-diazabicyclo[3.2.1]octanyl, cyclohexyl, (trifluoromethyl)piperazinyl, 7-azaspiro[3.5]nonanyl, piperazin-1-yl-2,2,3,3,5,5,6,6-d8, 2-azaspiro[3.5]nonanyl, 7-azaspiro[3.5]nonanyl, or pyrrolidinyl.

69. The compound of claim 54, or a pharmaceutically acceptable salt thereof, wherein G² is C₃₋₇ cycloalkyl.

70. The compound of claim 54, or a pharmaceutically acceptable salt thereof, wherein G² is cyclobutyl.

71. The compound of claim 54, or a pharmaceutically acceptable salt thereof, wherein G³ is —NR^{G}—or —O—.

72. The compound of claim 54, or a pharmaceutically acceptable salt thereof, wherein G⁴ is piperidinyl or piperazinyl.

73. The compound of claim 54, or a pharmaceutically acceptable salt thereof, wherein G⁴ is azetidinyl, piperidinyl, or piperazinyl.

74. The compound of claim 54, or a pharmaceutically acceptable salt thereof, wherein G⁴ is piperazinyl.

75. The compound of claim 54, or a pharmaceutically acceptable salt thereof, wherein a is 0.

76. The compound of claim 54, or a pharmaceutically acceptable salt thereof, wherein a is 1.

77. The compound of claim 54, or a pharmaceutically acceptable salt thereof, wherein b is 0.

78. The compound of claim 54, or a pharmaceutically acceptable salt thereof, wherein b is 1.

79. The compound of claim 54, or a pharmaceutically acceptable salt thereof, wherein c is 0.

80. The compound of claim 54, or a pharmaceutically acceptable salt thereof, wherein c is 1.

81. The compound of claim 54, or a pharmaceutically acceptable salt thereof, wherein d is 0.

82. The compound of claim 54, or a pharmaceutically acceptable salt thereof, wherein d is 1.

83. The compound of claim 54, or a pharmaceutically acceptable salt thereof, wherein e is 0.

84. The compound of claim 54, or a pharmaceutically acceptable salt thereof, wherein e is 1.

85. The compound of claim 54, or a pharmaceutically acceptable salt thereof, wherein f is 0.

86. The compound of claim 54, or a pharmaceutically acceptable salt thereof, wherein f is 1.

87. The compound of claim 54, or a pharmaceutically acceptable salt thereof, wherein g is 0.

88. The compound of claim 54, or a pharmaceutically acceptable salt thereof, wherein g is 1.

89. The compound of claim 54, or a pharmaceutically acceptable salt thereof, wherein R^{G} is H.

90. The compound of claim 1, or a pharmaceutically acceptable salt thereof, wherein E is an E3 ubiquitin ligase binding moiety that binds to cereblon.

91. The compound of claim 1, or a pharmaceutically acceptable salt thereof, wherein E is selected from the following:

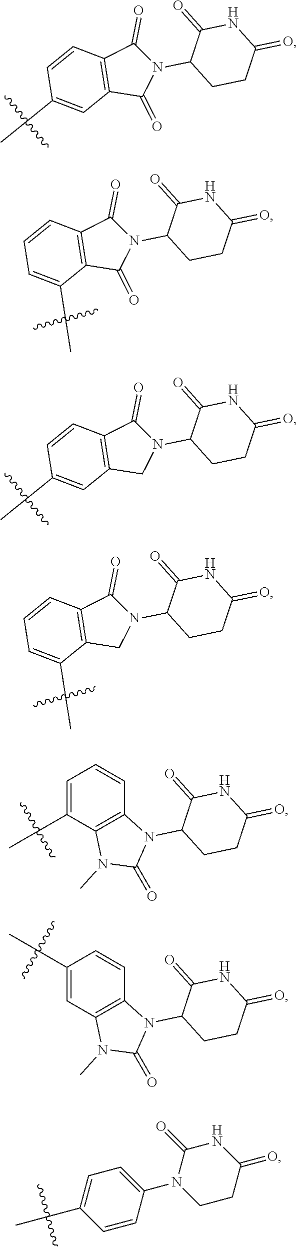

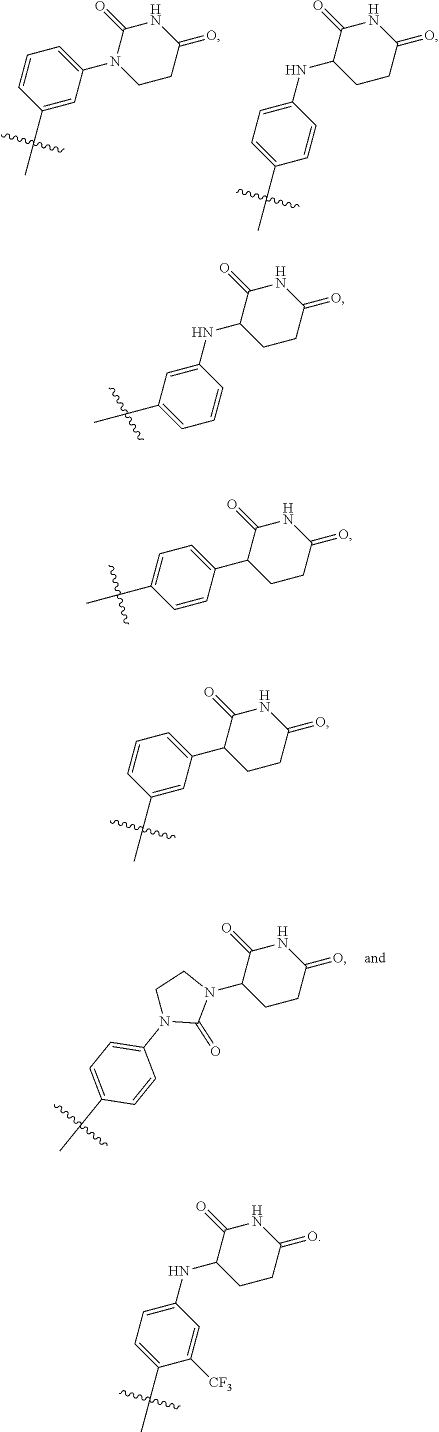

92. The compound of claim 1, or a pharmaceutically acceptable salt thereof, wherein E is selected from the following:

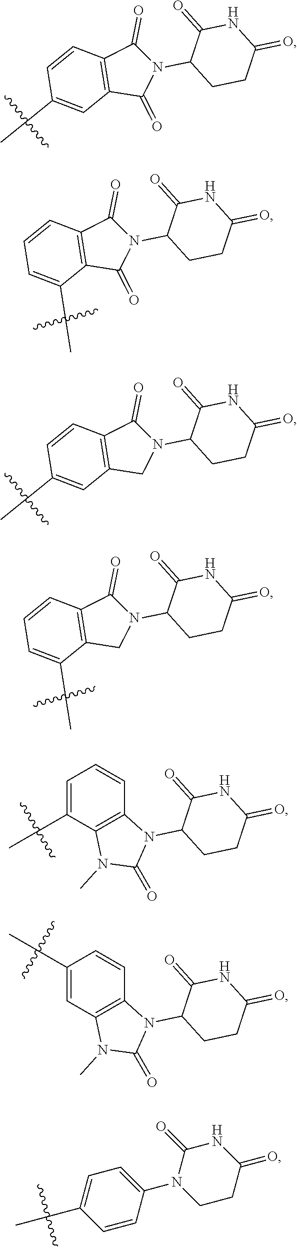

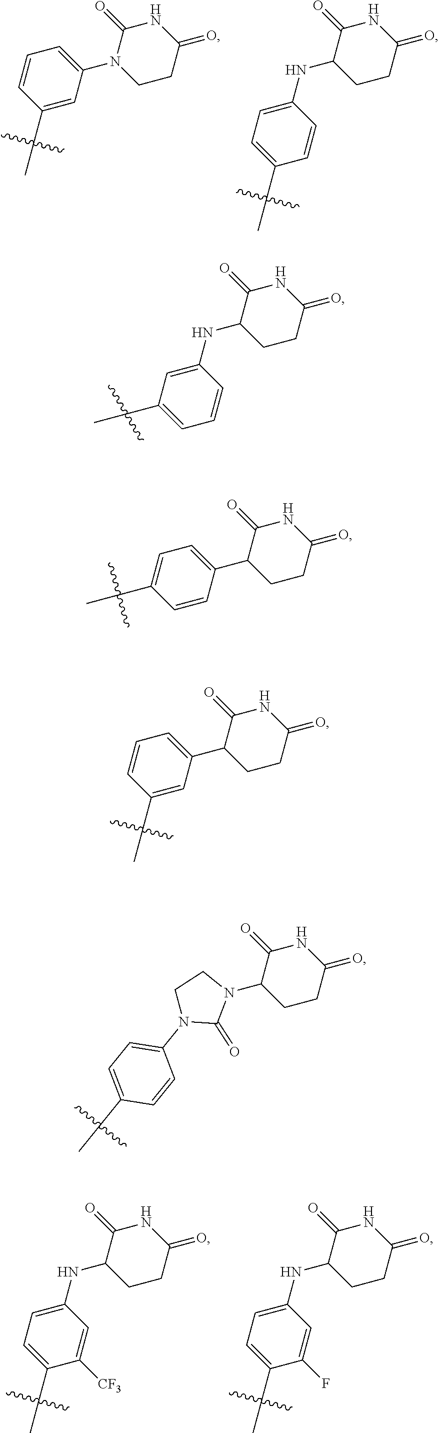

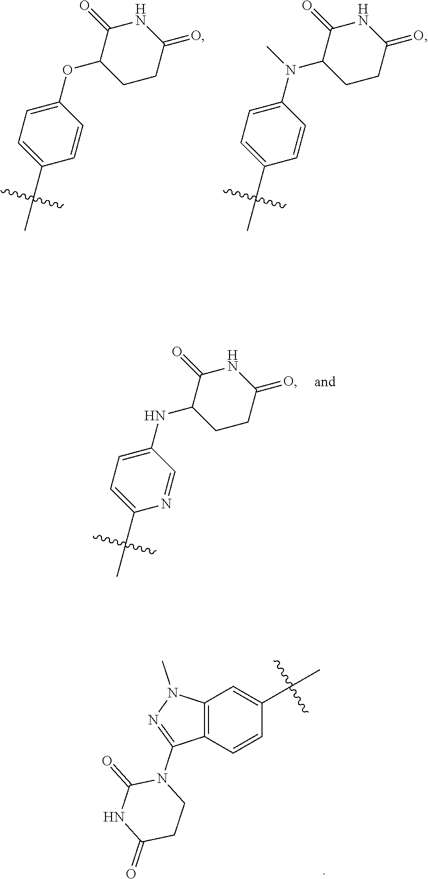 [SMILES: *c1ccc(OC2CCC(=O)NC2=O)cc1 ; *c1ccc(N(C)C2CCC(=O)NC2=O)cc1 ; *c1ccc(NC2CCC(=O)NC2=O)cn1 ; *c1ccc2c(N3CCC(=O)NC3=O)nn(C)c2c1]

93. The compound of claim 1, or a pharmaceutically acceptable salt thereof, wherein E is selected from the following:

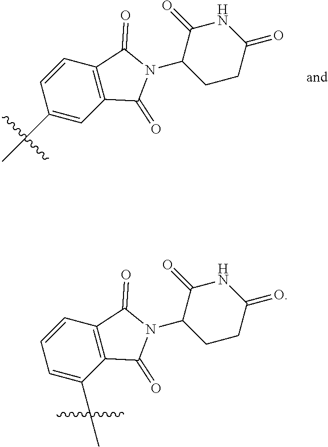 [SMILES: *c1ccc2c(c1)C(=O)N(C1CCC(=O)NC1=O)C2=O ; *c1cccc2c1C(=O)N(C1CCC(=O)NC1=O)C2=O]

94. The compound of claim 1, or a pharmaceutically acceptable salt thereof, wherein E is:

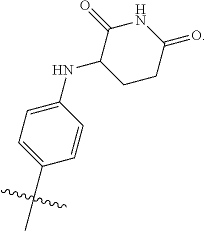 [SMILES: *c1ccc(NC2CCC(=O)NC2=O)cc1]

95. The compound of claim 1, or a pharmaceutically acceptable salt thereof, wherein E is selected from the following:

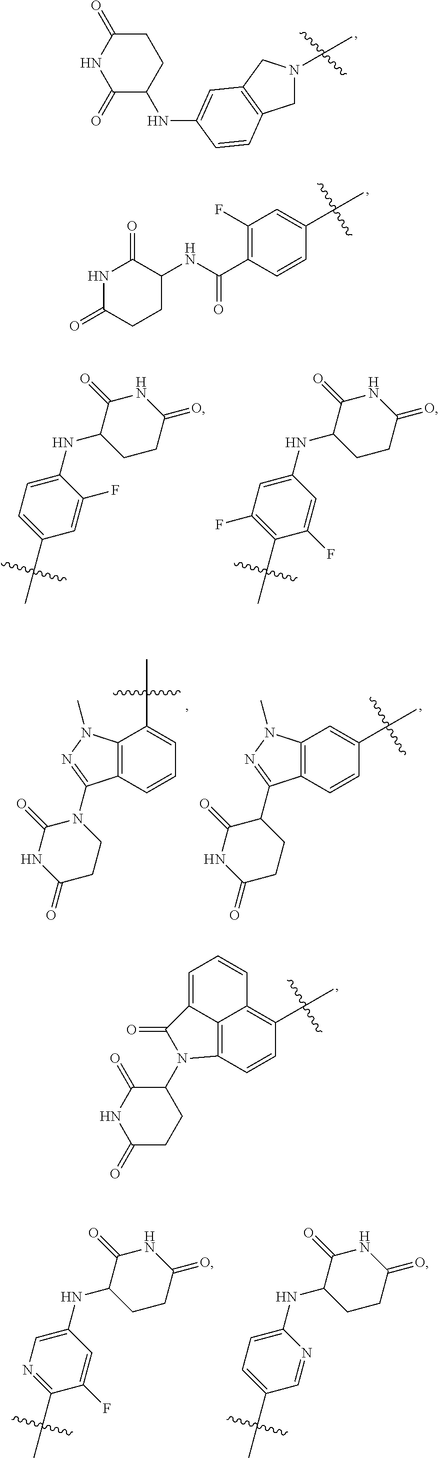

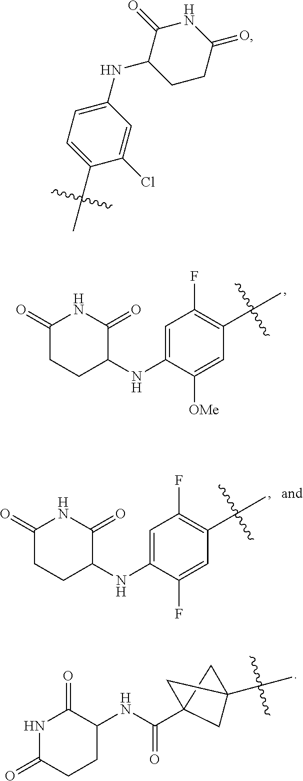 [SMILES: *c1ccc(NC2CCC(=O)NC2=O)cc1Cl ; *c1cc(OC)c(NC2CCC(=O)NC2=O)cc1F ; *c1cc(F)c(NC2CCC(=O)NC2=O)cc1F ; *C12CC(C(=O)NC3CCC(=O)NC3=O)(C1)C2]

96. The compound of claim 1, or a pharmaceutically acceptable salt thereof, wherein E is selected from the following:

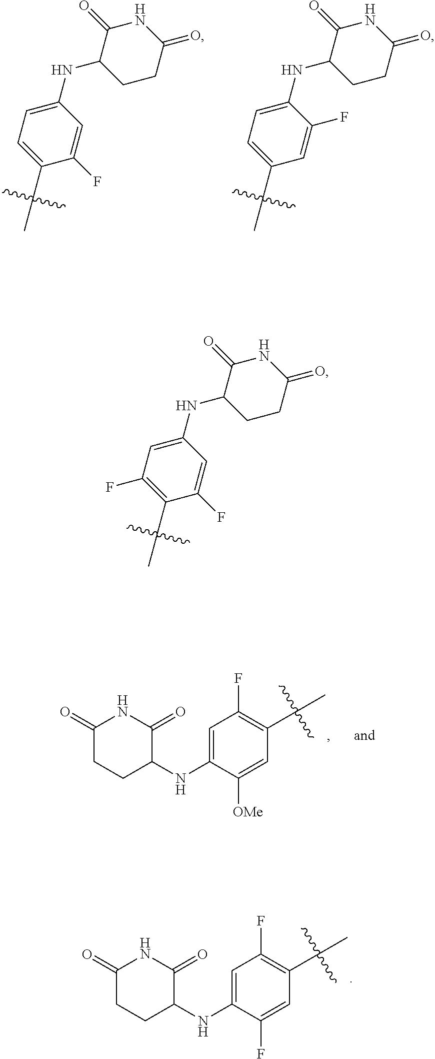 [SMILES: *c1ccc(NC2CCC(=O)NC2=O)cc1F ; *c1ccc(NC2CCC(=O)NC2=O)c(F)c1 ; *c1c(F)cc(NC2CCC(=O)NC2=O)cc1F ; *c1cc(OC)c(NC2CCC(=O)NC2=O)cc1F ; *c1cc(F)c(NC2CCC(=O)NC2=O)cc1F]

97. The compound of claim 1, or a pharmaceutically acceptable salt thereof, wherein E is selected from the following:

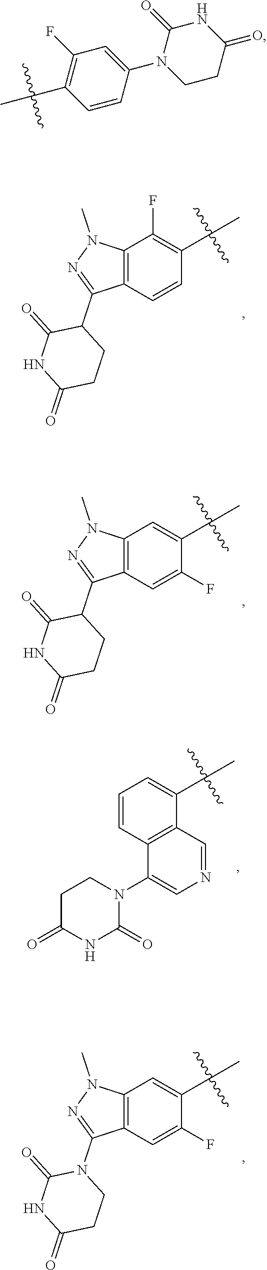 [SMILES: *c1ccc(N2CCC(=O)NC2=O)cc1F ; *c1ccc2c(C3CCC(=O)NC3=O)nn(C)c2c1F ; *c1cc2c(cc1F)c(C1CCC(=O)NC1=O)nn2C ; *c1cccc2c(N3CCC(=O)NC3=O)cncc12 ; *c1cc2c(cc1F)c(N1CCC(=O)NC1=O)nn2C]

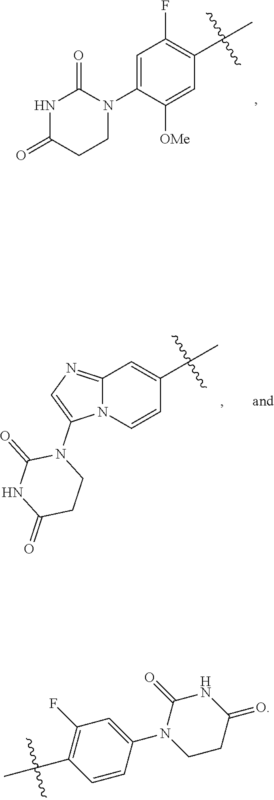 [SMILES: *c1cc(OC)c(N2CCC(=O)NC2=O)cc1F ; *c1ccn2c(N3CCC(=O)NC3=O)cnc2c1 ; *c1ccc(N2CCC(=O)NC2=O)cc1F]

98. The compound of claim 1, wherein the compound has Formula II: 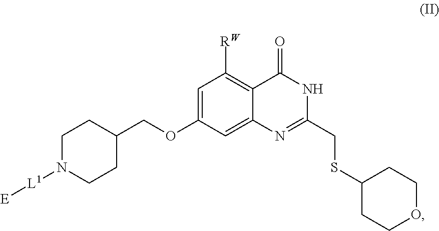
or a pharmaceutically acceptable salt thereof.

99. The compound of claim 1, wherein the compound has Formula III: 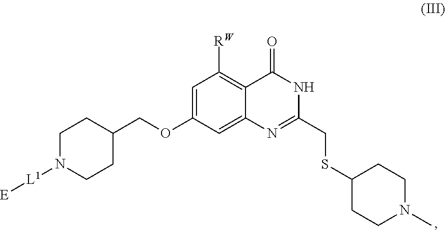
or a pharmaceutically acceptable salt thereof.

100. The compound of claim 1, wherein the compound is selected from the following:
N-(6-((2-(2,6-dioxopiperidin-3-yl)-1,3-dioxoisoindolin-4-yl)amino)hexyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(2-((2-(2,6-dioxopiperidin-3-yl)-1,3-dioxoisoindolin-4-yl)amino)ethyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(8-((2-(2,6-dioxopiperidin-3-yl)-1,3-dioxoisoindolin-4-yl)amino)octyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-4-((2-(2-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-3-oxopropoxy)ethoxy)ethyl)amino)isoindoline-1,3-dione;
2-(2,6-dioxopiperidin-3-yl)-4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)-3-oxopropyl)amino)isoindoline-1,3-dione;
2-(2,6-dioxopiperidin-3-yl)-4-((2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)amino)isoindoline-1,3-dione;
N-(2-(2-(2-((2-(2,6-dioxopiperidin-3-yl)-1,3-dioxoisoindolin-4-yl)amino)ethoxy)ethoxy)ethyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(4-((2-(2,6-dioxopiperidin-3-yl)-1,3-dioxoisoindolin-4-yl)amino)butyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-4-((9-(4-(((5-fluoro-4-oxo-2-(((tetrahydro-2H-pyran-4-yl)thio)methyl)-3,4-dihydroquinazolin-7-yl)oxy)methyl)piperidin-1-yl)-9-oxononyl)amino)isoindoline-1,3-dione;
2-(2,6-dioxopiperidin-3-yl)-4-((3-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-3-oxopropoxy)propyl)amino)isoindoline-1,3-dione;
2-(2,6-dioxopiperidin-3-yl)-4-((7-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-oxoheptyl)amino)isoindoline-1,3-dione;
2-(2,6-dioxopiperidin-3-yl)-4-((5-(4-(((5-fluoro-4-oxo-2-(((tetrahydro-2H-pyran-4-yl)thio)methyl)-3,4-dihydroquinazolin-7-yl)oxy)methyl)piperidin-1-yl)-5-oxopentyl)amino)isoindoline-1,3-dione;
N-(2-(3-((2-(2,6-dioxopiperidin-3-yl)-1,3-dioxoisoindolin-4-yl)amino)propoxy)ethyl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-5-((2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)amino)isoindoline-1,3-dione;
2-(2,6-dioxopiperidin-3-yl)-5-(4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperazin-1-yl)isoindoline-1,3-dione;
3-((4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)phenyl)amino)piperidine-2,6-dione;
3-(4-(4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperazin-1-yl)-1-oxoisoindolin-2-yl)piperidine-2,6-dione;
3-((4-(1-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperidin-4-yl)phenyl)amino)piperidine-2,6-dione;
1-(4-(1-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperidin-4-yl)phenyl)dihydropyrimidine-2,4(1H,3H)-dione;
2-(2,6-dioxopiperidin-3-yl)-4-(4-((4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperidin-1-yl)methyl)piperidin-1-yl)isoindoline-1,3-dione;
2-(2,6-dioxopiperidin-3-yl)-4-(4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperidin-4-yl)methyl)piperazin-1-yl)isoindoline-1,3-dione;
2-(2,6-dioxopiperidin-3-yl)-4-((2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethoxy)ethyl)amino)isoindoline-1,3-dione;
2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione;
2-(2,6-dioxopiperidin-3-yl)-4-(4-(((5-fluoro-4-oxo-2-(((tetrahydro-2H-pyran-4-yl)thio)methyl)-3,4-dihydroquinazolin-7-yl)oxy)methyl)piperidin-1-yl)isoindoline-1,3-dione;
N-(2-(2,6-dioxopiperidin-3-yl)-1,3-dioxoisoindolin-5-yl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
N-(2-(2,6-dioxopiperidin-3-yl)-1,3-dioxoisoindolin-4-yl)-2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetamide;
2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)isoindoline-1,3-dione;
2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)isoindoline-1,3-dione;
3-(5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)-1-oxoisoindolin-2-yl)piperidine-2,6-dione;
3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)-1-oxoisoindolin-2-yl)piperidine-2,6-dione;
3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)-3-methyl-2-oxo-2,3-dihydro-1H-benzo[d]imidazol-1-yl)piperidine-2,6-dione;
1-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)phenyl)dihydropyrimidine-2,4(1H,3H)-dione;
1-(3-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-yn-1-yl)phenyl)dihydropyrimidine-2,4(1H,3H)-dione;
2-(2,6-dioxopiperidin-3-yl)-5-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione;
2-(2,6-dioxopiperidin-3-yl)-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)isoindoline-1,3-dione;
2-(2,6-dioxopiperidin-3-yl)-5-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)isoindoline-1,3-dione;
2-(2,6-dioxopiperidin-3-yl)-4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)isoindoline-1,3-dione;
3-(4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)-1-oxoisoindolin-2-yl)piperidine-2,6-dione;
2-(2,6-dioxopiperidin-3-yl)-4-(4-((1-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)acetyl)piperidin-4-yl)methyl)piperazin-1-yl)isoindoline-1,3-dione;
2-(2,6-dioxopiperidin-3-yl)-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)isoindoline-1,3-dione;
2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)isoindoline-1,3-dione;
5-(3-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)-2-(2,6-dioxopiperidin-3-yl)isoindoline-1,3-dione;
(E)-2-(2,6-dioxopiperidin-3-yl)-5-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)prop-1-en-1-yl)isoindoline-1,3-dione;
2-(2,6-dioxopiperidin-3-yl)-5-(4-(((5-fluoro-2-(((1-methylpiperidin-4-yl)thio)methyl)-4-oxo-3,4-dihydroquinazolin-7-yl)oxy)methyl)piperidin-1-yl)isoindoline-1,3-dione;
3-(4-((4-(4-(((5-fluoro-4-oxo-2-(((tetrahydro-2H-pyran-4-yl)thio)methyl)-3,4-dihydroquinazolin-7-yl)oxy)methyl)piperidin-1-yl)but-2-yn-1-yl)amino)phenyl)piperidine-2,6-dione;
3-((4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutyl)methoxy)phenyl)amino)piperidine-2,6-dione;
3-({4-[3-({4-[({5-fluoro-2-[(oxan-4-ylsulfanyl)methyl]-4-oxo-3H-quinazolin-7-yl}oxy)methyl]piperidin-1-yl}methyl)cyclobutoxy]phenyl}amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-3-(trifluoromethyl)phenyl)amino)piperidine-2,6-dione;
3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)phenyl)amino)piperidine-2,6-dione;
3-((4-(3-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)phenyl)amino)piperidine-2,6-dione;
3-((4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)phenyl)amino)piperidine-2,6-dione; and
1-(3-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)phenyl)dihydropyrimidine-2,4(1H,3H)-dione;
or a pharmaceutically acceptable salt of any of the aforementioned.

101. The compound of any one of claims 1-3, wherein the compound is selected from the following:
3-((4-(4-(2-(3,3-difluoro-4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(4-(3-(((5-fluoro-4-oxo-2-(((1-(2,2,2-trifluoroethyl)piperidin-4-yl)thio)methyl)-3,4-dihydroquinazolin-7-yl)oxy)methyl)cyclobutyl)piperazin-1-yl)phenyl)amino)piperidine-2,6-dione;
7-(((1s,3s)-3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one;
7-(((1r,3r)-3-(4-(4-aminophenyl)piperazin-1-yl)cyclobutyl)methoxy)-5-fluoro-2-(((1-(2,2,2-trifluoroethyl)piperidin-4-yl)thio)methyl)quinazolin-4(3H)-one;
5-fluoro-3-(4-methoxybenzyl)-7-((1-(2-(4-nitrophenoxy)ethyl)piperidin-4-yl)methoxy)-2-(((tetrahydro-2H-pyran-4-yl)thio)methyl)quinazolin-4(3H)-one;
(S)-3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
(R)-3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-(5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-oxoisoindolin-2-yl)piperidine-2, 6-dione;
3-(4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-oxoisoindolin-2-yl)piperidine-2,6-dione;
2-(2,6-dioxopiperidin-3-yl)-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)isoindoline-1,3-dione;
2-(2,6-dioxopiperidin-3-yl)-5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)isoindoline-1,3-dione;
3-((3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-(4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenoxy)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)(methyl)amino)piperidine-2,6-dione;
3-((3-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)amino)piperidine-2,6-dione;
3-((4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione;
3-((6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)pyridin-3-yl)amino)piperidine-2,6-dione;
1-(4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)dihydropyrimidine-2,4(1H,3H)-dione;
3-((4-(3-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,6-diazabicyclo[3.1.1]heptan-6-yl)phenyl)amino)piperidine-2,6-dione;
2-(2,6-dioxopiperidin-3-yl)-5-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)ethynyl)isoindoline-1,3-dione;
3-((4-(6-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,6-diazabicyclo[3.1.1]heptan-3-yl)phenyl)amino)piperidine-2,6-dione;
1-(6-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
1-(6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)dihydropyrimidine-2,4(1H,3H)-dione;
3-((4-(((3aR,5r,6aS)-2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)octahydrocyclopenta[c]pyrrol-5-yl)oxy)phenyl)amino)piperidine-2,6-dione;
1-(4-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)dihydropyrimidine-2,4(1H,3H)-dione;
3-(5-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-3-methyl-2-oxo-2,3-dihydro-1H-benzo[d]imidazol-1-yl)piperidine-2,6-dione;
2-(2,6-dioxopiperidin-3-yl)-5-((1r,3r)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
2-(2,6-dioxopiperidin-3-yl)-5-((1s,3s)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
2-(2,6-dioxopiperidin-3-yl)-4-((1r,3r)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
2-(2,6-dioxopiperidin-3-yl)-4-((1s,3s)-3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutoxy)isoindoline-1,3-dione;
3-((4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)cyclobutoxy)phenyl) amino)piperidine-2,6-dione;
3-((4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione;
3-((4-(4-(2-(4-(((5-fluoro-2-((((1r,4r)-4-hydroxycyclohexyl)thio)methyl)-4-oxo-3,4-dihydroquinazolin-7-yl)oxy)methyl)piperidin-1-yl)ethyl)piperazin-1-yl)phenyl)amino)piperidine-2,6-dione;
3-((4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)phenyl)amino)piperidine-2,6-dione;
2-(2,6-dioxopiperidin-3-yl)-5-(4-(4-((5-fluoro-4-oxo-2-(((tetrahydro-2H-pyran-4-yl)thio)methyl)-3,4-dihydroquinazolin-7-yl)methyl)piperazin-1-yl)piperidin-1-yl)isoindoline-1,3-dione;
3-((4-(4-(4-(2-(5-fluoro-4-oxo-2-(((tetrahydro-2H-pyran-4-yl)thio)methyl)-3,4-dihydroquinazolin-7-yl)ethyl)piperazin-1-yl)piperidin-1-yl)phenyl)amino)piperidine-2,6-dione;
3-((4-((1-(4-(((5-fluoro-4-oxo-2-(((tetrahydro-2H-pyran-4-yl)thio)methyl)-3,4-dihydroquinazolin-7-yl)oxy)methyl)cyclohexyl)piperidin-4-yl)oxy)phenyl)amino)piperidine-2,6-dione;
3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((4-(4-(((5-fluoro-4-oxo-2-(((1-(2,2,2-trifluoroethyl)piperidin-4-yl)thio)methyl)-3,4-dihydroquinazolin-7-yl)oxy)methyl)-[1,4′-bipiperidin]-1′-yl)phenyl)amino)piperidine-2,6-dione; and
3-((4-(3-((4-(((5-fluoro-2-((((1r,4r)-4-hydroxycyclohexyl)thio)methyl)-4-oxo-3,4-dihydroquinazolin-7-yl)oxy)methyl)piperidin-1-yl)methyl)cyclobutoxy)phenyl)amino)piperidine-2,6-dione,
or a pharmaceutically acceptable salt of any of the aforementioned.

102. The compound of claim 1, wherein the compound is selected from:
3-(5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-3-methyl-2-oxo-2,3-dihydro-1H-benzo[d]imidazol-1-yl)piperidine-2,6-dione;
3-((2-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)isoindolin-5-yl)amino)piperidine-2,6-dione;
N-(2,6-dioxopiperidin-3-yl)-2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)benzamide;
3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)amino)phenyl)amino)piperidine-2,6-dione;
3-((4-(8-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,8-diazabicyclo[3.2.1]octan-3-yl)phenyl)amino)piperidine-2,6-dione;
3-((4-(4-(2-(4-(((2-(cyclopentylmethyl)-5-fluoro-4-oxo-3,4-dihydroquinazolin-7-yl)oxy)methyl)piperidin-1-yl)ethyl)piperazin-1-yl)phenyl)amino)piperidine-2,6-dione;
3-((2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3,5-difluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione;
1-(7-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)dihydropyrimidine-2,4(1H,3H)-dione;
3-(6-(4-(2-(4-(((5-Fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)piperidine-2,6-dione;
3-(6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-2-oxobenzo[cd]indol-1(2H)-yl)piperidine-2,6-dione;
3-((4-fluoro-3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(4-(2-(4-(((2-(cyclopropylmethyl)-5-fluoro-4-oxo-3,4-dihydroquinazolin-7-yl)oxy)methyl)piperidin-1-yl)ethyl)piperazin-1-yl)-3-fluorophenyl)amino)piperidine-2,6-dione;
3-((4-(4-(2-(4-(((2-(cyclobutylmethyl)-5-fluoro-4-oxo-3,4-dihydroquinazolin-7-yl)oxy)methyl)piperidin-1-yl)ethyl)piperazin-1-yl)-3-fluorophenyl)amino)piperidine-2,6-dione 3-((4-(4-(2-(4-(((2-(cyclopentylmethyl)-5-fluoro-4-oxo-3,4-dihydroquinazolin-7-yl)oxy)methyl)piperidin-1-yl)ethyl)piperazin-1-yl)-3-fluorophenyl)amino)piperidine-2,6-dione;
3-((5-fluoro-6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)pyridin-3-yl)amino)piperidine-2,6-dione;
3-((4-(3-((4-(((5-fluoro-4-oxo-2-(((1-(2,2,2-trifluoroethyl)piperidin-4-yl)thio)methyl)-3,4-dihydroquinazolin-7-yl)oxy)methyl)piperidin-1-yl)methyl)cyclobutoxy)phenyl)amino)piperidine-2,6-dione;
3-((4-(3-((4-(((5-fluoro-4-oxo-2-(2-(tetrahydro-2H-pyran-4-yl)ethyl)-3,4-dihydroquinazolin-7-yl)oxy)methyl)piperidin-1-yl)methyl)cyclobutoxy)phenyl)amino)piperidine-2,6-dione;
3-((4-((4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclohexyl)oxy)phenyl)amino)piperidine-2,6-dione;
3-((4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione;
3-((4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidin]-1′-yl)phenyl)amino)piperidine-2,6-dione;
3-((4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidin]-1′-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-1-yl)phenyl)amino)piperidine-2,6-dione;
3-((5-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)pyridin-2-yl)amino)piperidine-2,6-dione;
3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)oxy)phenyl)amino)piperidine-2,6-dione;
3-((4-(3-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3,8-diazabicyclo[3.2.1]octan-8-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-2-(trifluoromethyl)piperazin-1-yl)phenyl)amino)piperidine-2,6-dione;
3-((4-((1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)(methyl)amino)phenyl)amino)piperidine-2,6-dione;
3-((4-(4-(4-(((5-fluoro-4-oxo-2-(((tetrahydro-2H-pyran-4-yl)thio)methyl)-3,4-dihydroquinazolin-7-yl)oxy)methyl)cyclohexyl)piperazin-1-yl)phenyl)amino)piperidine-2,6-dione;
3-((4-(4-(4-(2-((5-fluoro-4-oxo-2-(((tetrahydro-2H-pyran-4-yl)thio)methyl)-3,4-dihydroquinazolin-7-yl)oxy)ethyl)piperazin-1-yl)piperidin-1-yl)phenyl)amino)piperidine-2,6-dione;
3-((3-fluoro-4-(4-(1-(2-((5-fluoro-4-oxo-2-(((tetrahydro-2H-pyran-4-yl)thio)methyl)-3,4-dihydroquinazolin-7-yl)oxy)ethyl)piperidin-4-yl)piperazin-1-yl)phenyl)amino)piperidine-2,6-dione;
3-((4-(3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidin-1-yl)phenyl)amino)piperidine-2,6-dione;
3-((4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-azaspiro[3.5]nonan-7-yl)phenyl)amino)piperidine-2,6-dione;
3-((4-(4-(2-(4-(((5,6-difluoro-4-oxo-2-(((tetrahydro-2H-pyran-4-yl)thio)methyl)-3,4-dihydroquinazolin-7-yl)amino)methyl)piperidin-1-yl)ethyl)piperazin-1-yl)phenyl)amino)piperidine-2,6-dione;
3-((4-(3-(4-(2-((5-fluoro-4-oxo-2-(((tetrahydro-2H-pyran-4-yl)thio)methyl)-3,4-dihydroquinazolin-7-yl)oxy)ethyl)piperazin-1-yl)azetidin-1-yl)phenyl)amino)piperidine-2,6-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl-2,2,3,3,5,5,6,6-d8)phenyl)amino)piperidine-2,6-dione;
3-((4-(5-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)pyridin-2-yl)phenyl)amino)piperidine-2,6-dione;
3-((4-(6-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)pyridin-3-yl)phenyl)amino)piperidine-2,6-dione;
N-(2,6-dioxopiperidin-3-yl)-3-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)bicyclo[1.1.1]pentane-1-carboxamide;
3-((3-fluoro-4-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)phenyl)amino)piperidine-2,6-dione;
3-((3-chloro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((4-(7-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-azaspiro[3.5]nonan-2-yl)phenyl)amino)piperidine-2,6-dione;
3-((5-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-2-methoxyphenyl)amino)piperidine-2,6-dione;
3-((5-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-2-methoxyphenyl)amino)piperidine-2,6-dione;
3-((2,5-difluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
3-((3-chloro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-7-azaspiro[3.5]nonan-7-yl)phenyl)amino)piperidine-2,6-dione;
3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)phenyl)amino)piperidine-2,6-dione;
3-((3-fluoro-4-(3-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)azetidin-1-yl)phenyl)amino)piperidine-2,6-dione;
3-((3-fluoro-4-(4-(3-(4-(((5-fluoro-4-oxo-2-(2-(tetrahydro-2H-pyran-4-yl)ethyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)(methyl)amino)phenyl)amino)piperidine-2,6-dione;
3-((3-fluoro-4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)-[1,3′-biazetidin]-1′-yl)phenyl)amino)piperidine-2,6-dione; and
N-(2,6-dioxopiperidin-3-yl)-2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)benzamide, or a pharmaceutically acceptable salt of any of the aforementioned.

103. The compound of claim 1, wherein the compound is selected from:
3-((3-fluoro-4-((3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)cyclobutyl)amino)phenyl)amino)piperidine-2,6-dione;
N-(2,6-dioxopiperidin-3-yl)-2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)benzamide;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidine-1-carbonyl)piperidin-1-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperidin-1-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)piperazin-1-yl)phenyl)amino)piperidine-2,6-dione;
1-(6-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
3-(6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-1-methyl-1H-indazol-3-yl)piperidine-2,6-dione;
1-(3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)dihydropyrimidine-2,4(1H,3H)-dione;
1-(3-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)dihydropyrimidine-2,4(1H,3H)-dione;
1-(7-fluoro-6-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
1-(5-fluoro-6-(4-(((5-fluoro-4-oxo-2-(((tetrahydro-2H-pyran-4-yl)oxy)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
1-(6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-1-methyl-1H-indazol-3-yl)dihydropyrimidine-2,4(1H,3H)-dione;
1-(8-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)isoquinolin-4-yl)dihydropyrimidine-2,4(1H,3H)-dione;
1-(5-fluoro-6-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)-1-methyl-1H-indazol-3-yl)dihydropyrimidine-2,4(1H,3H)-dione;
1-(5-fluoro-6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-1-methyl-1H-indazol-3-yl)dihydropyrimidine-2,4(1H,3H)-dione;
1-(4-fluoro-3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)dihydropyrimidine-2,4(1H,3H)-dione;
1-(5-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-2-methoxyphenyl)dihydropyrimidine-2,4(1H,3H)-dione;
1-(7-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)imidazo[1,2-a]pyridin-3-yl)dihydropyrimidine-2,4(1H,3H)-dione;
1-(7-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidin-1-yl)piperidin-1-yl)imidazo[1,2-a]pyridin-3-yl)dihydropyrimidine-2,4(1H,3H)-dione;
3-(6-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-2-oxobenzo[cd]indol-1(2H)-yl)piperidine-2,6-dione;
3-((2-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)-4-hydroxypiperidin-1-yl)phenyl)amino)piperidine-2,6-dione;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)azetidin-1-yl)piperidin-1-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidine]-1′-carbonyl)piperidin-1-yl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)piperazin-1-yl)phenyl)amino)piperidine-2,6-dione;
3-((3-fluoro-4-(4-(4-(((5-fluoro-4-oxo-2-(2-(tetrahydro-2H-pyran-4-yl)ethyl)-3,4-dihydroquinazolin-7-yl)oxy)methyl)-[1,4′-bipiperidine]-1′-carbonyl)piperidin-1-yl)phenyl)amino)piperidine-2,6-dione;
3-((3-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)pyrrolidine-1-carbonyl)piperidin-1-yl)phenyl)amino)piperidine-2,6-dione;
3-((3-fluoro-4-(4-(3-(4-(((5-fluoro-4-oxo-2-(((tetrahydro-2H-pyran-4-yl)oxy)methyl)-3,4-dihydroquinazolin-7-yl)oxy)methyl)piperidin-1-yl)azetidine-1-carbonyl)piperidin-1-yl)phenyl)amino)piperidine-2,6-dione;
3-((4-(4-(3-(4-(((2-(cyclopropylmethyl)-5-fluoro-4-oxo-3,4-dihydroquinazolin-7-yl)oxy)methyl)piperidin-1-yl)azetidine-1-carbonyl)piperidin-1-yl)-3-fluorophenyl)amino)piperidine-2,6-dione;
3-((3-fluoro-4-(4-(3-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)-2-oxoethyl)azetidin-1-yl)piperidin-1-yl)phenyl)amino)piperidine-2,6-dione;
1-(3-fluoro-4-(4-((4-(((5-fluoro-4-oxo-2-(((tetrahydro-2H-pyran-4-yl)oxy)methyl)-3,4-dihydroquinazolin-7-yl)oxy)methyl)piperidin-1-yl)methyl)piperidin-1-yl)phenyl)dihydropyrimidine-2,4(1H,3H)-dione;
1-(6-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione;
1-(5-fluoro-6-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione;
1-(5-fluoro-6-(4-((4-(((5-fluoro-4-oxo-2-(((tetrahydro-2H-pyran-4-yl)oxy)methyl)-3,4-dihydroquinazolin-7-yl)oxy)methyl)piperidin-1-yl)methyl)piperidin-1-yl)-1-methyl-1H-indazol-3-yl)dihydropyrimidine-2,4(1H,3H)-dione;
1-(6-(4-(((5-fluoro-4-oxo-2-(((tetrahydro-2H-pyran-4-yl)oxy)methyl)-3,4-dihydroquinazolin-7-yl)oxy)methyl)piperidin-1-yl)-1-methyl-1H-indazol-3-yl)dihydropyrimidine-2,4(1H,3H)-dione;
1-(5-fluoro-6-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)-1-methyl-1H-indazol-3-yl)dihydropyrimidine-2,4(1H,3H)-dione;
1-(6-(1-(2-((5-fluoro-4-oxo-2-(((tetrahydro-2H-pyran-4-yl)thio)methyl)-3,4-dihydroquinazolin-7-yl)oxy)ethyl)piperidin-4-yl)-1-methyl-1H-indazol-3-yl)dihydropyrimidine-2,4(1H,3H)-dione;
1-(6-(1′-(2-((5-fluoro-4-oxo-2-(((tetrahydro-2H-pyran-4-yl)thio)methyl)-3,4-dihydroquinazolin-7-yl)oxy)ethyl)-[1,4′-bipiperidin]-4-yl)-1-methyl-1H-indazol-3-yl)dihydropyrimidine-2,4(1H,3H)-dione;
3-((4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazine-1-carbonyl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)acetyl)piperazin-1-yl)phenyl)amino)piperidine-2,6-dione;
2-(2,6-dioxopiperidin-3-yl)-5-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)isoindoline-1,3-dione;
2-(2,6-dioxopiperidin-3-yl)-5-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)isoindoline-1,3-dione;
3-((2-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-(2-(3-(((5-fluoro-4-oxo-2-(((tetrahydro-2H-pyran-4-yl)thio)methyl)-3,4-dihydroquinazolin-7-yl)oxy)methyl)azetidin-1-yl)ethyl)piperazin-1-yl)phenyl)amino)piperidine-2,6-dione;
3-({4-[4-(2-{4-[({2-[(cyclohexylsulfanyl)methyl]-5-fluoro-4-oxo-3H-quinazolin-7-yl}oxy)methyl]piperidin-1-yl}ethyl)piperazin-1-yl]-3-fluorophenyl}amino)piperidine-2,6-dione;
3-(5-fluoro-6-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-1-methyl-1H-indazol-3-yl)piperidine-2,6-dione;
1-(3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)dihydropyrimidine-2,4(1H,3H)-dione;
1-(3-fluoro-4-(4-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperazin-1-yl)phenyl)dihydropyrimidine-2,4(1H,3H)-dione;
1-(5-fluoro-6-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
(3S)—N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)pyrrolidine-3-carboxamide;
(3R)—N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)pyrrolidine-3-carboxamide;
N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidine-4-carboxamide;
N-(4-((2,6-dioxopiperidin-3-yl)amino)-2-fluorophenyl)-1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)-N-methylpiperidine-4-carboxamide;
1-(7-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
1-(3-fluoro-4-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)phenyl)dihydropyrimidine-2,4(1H,3H)-dione;
1-(7-fluoro-6-(1-(2-(4-(((5-fluoro-4-oxo-2-(((tetrahydro-2H-pyran-4-yl)oxy)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
3-((3-fluoro-4-(4-(2-(4-(((4-oxo-2-(((tetrahydro-2H-pyran-4-yl)thio)methyl)-3,4-dihydroquinazolin-7-yl)oxy)methyl)piperidin-1-yl)ethyl)piperazin-1-yl)phenyl)amino)piperidine-2,6-dione;
1-(5-fluoro-6-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)-1-methyl-1H-indazol-3-yl)dihydropyrimidine-2,4(1H,3H)-dione;
1-(3-fluoro-4-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)phenyl)dihydropyrimidine-2,4(1H,3H)-dione;
1-(7-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)imidazo[1,2-a]pyridin-3-yl)dihydropyrimidine-2,4(1H,3H)-dione;
1-(8-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)isoquinolin-4-yl)dihydropyrimidine-2,4(1H,3H)-dione;
1-(7-(1-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperidin-4-yl)imidazo[1,2-a]pyridin-3-yl)dihydropyrimidine-2,4(1H,3H)-dione;
1-(5-fluoro-6-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)piperazin-1-yl)-1-methyl-1H-indazol-3-yl)dihydropyrimidine-2,4(1H,3H)-dione;
3-((3-fluoro-4-(4-((1-(2-((5-fluoro-4-oxo-2-(((tetrahydro-2H-pyran-4-yl)thio)methyl)-3,4-dihydroquinazolin-7-yl)oxy)ethyl)piperidin-4-yl)methyl)piperazin-1-yl)phenyl)amino)piperidine-2,6-dione;
3-((3-fluoro-4-(4-(((5-fluoro-4-oxo-2-(((tetrahydro-2H-pyran-4-yl)thio)methyl)-3,4-dihydroquinazolin-7-yl)oxy)methyl)-2-oxo-[1,4′:1′,4″-terpiperidin]-1″-yl)phenyl)amino)piperidine-2,6-dione;
3-((3-fluoro-4-(9-(2-((5-fluoro-4-oxo-2-(((tetrahydro-2H-pyran-4-yl)thio)methyl)-3,4-dihydroquinazolin-7-yl)oxy)ethyl)-3,9-diazaspiro[5.5]undecan-3-yl)phenyl)amino)piperidine-2,6-dione;
3-((2-fluoro-4-(4-(2-(4-(((5-fluoro-4-oxo-2-(2-(tetrahydro-2H-pyran-4-yl)ethyl)-3,4-dihydroquinazolin-7-yl)oxy)methyl)piperidin-1-yl)ethyl)piperazin-1-yl)phenyl)amino)piperidine-2,6-dione;
3-((3-fluoro-4-(4-((3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)methyl)piperidin-1-yl)phenyl)amino)piperidine-2,6-dione;
3-((2,5-difluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((4-(4-(((2-(cyclopropylmethyl)-5-fluoro-4-oxo-3,4-dihydroquinazolin-7-yl)oxy)methyl)-[1,4′-bipiperidin]-1′-yl)-3-fluorophenyl)amino)piperidine-2,6-dione;
3-((3-fluoro-4-(4-((4-((5-fluoro-4-oxo-2-(((tetrahydro-2H-pyran-4-yl)thio)methyl)-3,4-dihydroquinazolin-7-yl)ethynyl)piperidin-1-yl)methyl)piperidin-1-yl)phenyl)amino)piperidine-2,6-dione;
3-((2-fluoro-4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((2-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)phenyl)amino)piperidine-2,6-dione;
3-((3-fluoro-4-(4-(2-((5-fluoro-4-oxo-2-(((tetrahydro-2H-pyran-4-yl)thio)methyl)-3,4-dihydroquinazolin-7-yl)oxy)ethyl)-[1,4′-bipiperidin]-1′-yl)phenyl)amino)piperidine-2,6-dione;
3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)-[1,4′-bipiperidin]-1′-yl)phenyl)amino)piperidine-2,6-dione;
3-((3-fluoro-4-(4-(4-(((5-fluoro-4-oxo-2-(((tetrahydro-2H-pyran-4-yl)thio)methyl)-3,4-dihydroquinazolin-7-yl)oxy)methyl)piperidine-1-carbonyl)-[1,4′-bipiperidin]-1′-yl)phenyl)amino)piperidine-2,6-dione;
3-((3-fluoro-4-(4-(2-(4-((5-fluoro-4-oxo-2-(((tetrahydro-2H-pyran-4-yl)thio)methyl)-3,4-dihydroquinazolin-7-yl)ethynyl)piperidin-1-yl)ethyl)piperazin-1-yl)phenyl)amino)piperidine-2,6-dione;
3-((3-fluoro-4-(6-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)-2-azaspiro[3.3]heptan-2-yl)phenyl)amino)piperidine-2,6-dione;
3-((3-fluoro-4-(4-(((5-fluoro-4-oxo-2-(((tetrahydro-2H-pyran-4-yl)thio)methyl)-3,4-dihydroquinazolin-7-yl)oxy)methyl)-[1,4′:1′,4″-terpiperidin]-1″-yl)phenyl)amino)piperidine-2,6-dione;
3-((5-fluoro-4-(4-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)piperidin-1-yl)-2-methoxyphenyl)amino)piperidine-2,6-dione;
3-((4-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)-3-(trifluoromethyl)phenyl)amino)piperidine-2,6-dione;
3-((3-fluoro-4-(4-((4-(((5-fluoro-4-oxo-2-(((tetrahydro-2H-pyran-4-yl)thio)methyl)-3,4-dihydroquinazolin-7-yl)oxy)methyl)-[1,4′-bipiperidin]-1′-yl)methyl)piperidin-1-yl)phenyl)amino)piperidine-2,6-dione;
3-((3-fluoro-4-(4-(3-((4-(((5-fluoro-4-oxo-2-(((tetrahydro-2H-pyran-4-yl)thio)methyl)-3,4-dihydroquinazolin-7-yl)oxy)methyl)piperidin-1-yl)methyl)azetidin-1-yl)piperidin-1-yl)phenyl)amino)piperidine-2,6-dione;
3-((3-fluoro-4-(4-(3-(4-(((4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-((3-fluoro-4-(4-(2-(4-(((5-fluoro-4-oxo-2-(((tetrahydro-2H-pyran-4-yl)thio)methyl)-3,4-dihydroquinazolin-7-yl)oxy)methyl)piperidin-1-yl)ethyl)-3-oxopiperazin-1-yl)phenyl)amino)piperidine-2,6-dione;
3-((3-fluoro-4-(4-(3-(4-(2-((5-fluoro-4-oxo-2-(((tetrahydro-2H-pyran-4-yl)thio)methyl)-3,4-dihydroquinazolin-7-yl)oxy)ethyl)piperidin-1-yl)azetidin-1-yl)piperidin-1-yl)phenyl)amino)piperidine-2,6-dione;
3-((4-fluoro-3-(4-(3-(4-(((5-fluoro-4-oxo-2-(((tetrahydro-2H-pyran-4-yl)thio)methyl)-3,4-dihydroquinazolin-7-yl)oxy)methyl)piperidin-1-yl)azetidin-1-yl)piperidin-1-yl)phenyl)amino)piperidine-2,6-dione;
3-({4-[4-(3-{4-[({2-[(cyclohexylsulfanyl)methyl]-5-fluoro-4-oxo-3H-quinazolin-7-yl}oxy)methyl]piperidin-1-yl}azetidin-1-yl)piperidin-1-yl]-3-fluorophenyl}amino)piperidine-2,6-dione,
or a pharmaceutically acceptable salt of any of the aforementioned.

104. A pharmaceutical composition comprising a compound of claim 1, or a pharmaceutically acceptable salt thereof, and at least one pharmaceutically acceptable carrier.

105. A method of degrading PARP14, comprising contacting a compound of claim 1, or a pharmaceutically acceptable salt thereof, with said PARP14.

106. A method of treating cancer in a patient in need of treatment comprising administering to said patient a therapeutically effective amount of a compound of claim 1, or a pharmaceutically acceptable salt thereof.

107. A method of treating cancer in a patient in need of treatment comprising administering to said patient a therapeutically effective amount of a compound of claim 1, or a pharmaceutically acceptable salt thereof, in combination with an additional therapy or therapeutic agent.

108. The method of claim 106 wherein said cancer is multiple myeloma, DLBCL, hepatocellular carcinoma, bladder cancer, esophageal cancer, head and neck cancer, kidney cancer, prostate cancer, rectal cancer, stomach cancer, thyroid cancer, uterine cancer, breast cancer, glioma, follicular lymphoma, pancreatic cancer, lung cancer, colon cancer, or melanoma.

109. A method of treating an inflammatory disease in a patient in need of treatment comprising administering to said patient a therapeutically effective amount of a compound of claim 1, or a pharmaceutically acceptable salt thereof.

110. The method of claim 109, wherein the inflammatory disease is selected from asthma, atopic dermatitis, psoriasis, rhinitis, systemic sclerosis, keloids, an eosinophilic disorder, pulmonary fibrosis, and a type 2 cytokine pathology.

111. The method of claim 109, wherein the inflammatory disease is atopic dermatitis.

112. The method of claim 109, wherein the inflammatory disease is systemic sclerosis.

113. The method of claim 110, wherein said asthma is steroid-insensitive asthma, steroid-refractory asthma, steroid-resistant asthma, atopic asthma, nonatopic asthma, persistent asthma, severe asthma, or steroid-refractory severe asthma.

114. The method of claim 113, wherein said severe asthma is T2 high endotype, T2 low endotype, or non-T2 endotype.

115. The method of claim 113, wherein said severe asthma is T2 low endotype or non-T2 endotype.
